(19) United States
(12) Patent Application Publication
(10) Pub. No.: US 2010/0179213 A1
(43) Pub. Date: Jul. 15, 2010

(54) Methods and Compositions Involving miRNAs In Cancer Stem Cells

(75) Inventors: Lubna Patrawala, Cambridge, MA (US); Dean G. Tang, Austin, TX (US); Kevin Kelnar, Kyle, TX (US); Jason Wiggins, Austin, TX (US); Stephanie Volz, Austin, TX (US); Jeffrey Shelton, Buda, TX (US); Can Liu, Austin, TX (US); Andreas G. Bader, Austin, TX (US); David Brown, Austin, TX (US)

Correspondence Address: Fulbright & Jaworski L.L.P., 600 Congress Avenue, Suite 2400, Austin, TX, 78701, US

(73) Assignees: MIRNA THERAPEUTICS, INC., Austin, TX (US); The Board of Regents of the University of Texas System, Austin, TX (US)

(21) Appl. No.: 12/616,616

(22) Filed: Nov. 11, 2009

Related U.S. Application Data
(60) Provisional application No. 61/113,385, filed on Nov. 11, 2008.

(51) Int. Cl. A61K 31/7088; A61P 35/04; C12Q 1/68

(52) U.S. Cl. 514/44 R; 435/6

(57) ABSTRACT

The present invention concerns methods and compositions for treating a patient having, suspected of having, or at risk of developing cancer by targeting cancer stem cells.

This application claims priority to U.S. Provisional Patent application Ser. No. 61/113,385 filed on Nov. 11, 2008, which is incorporated herein by reference in its entirety.

This invention was made with government support under W81XWH-07-1-0616 awarded by the Department of Defense. The government has certain rights in the invention.

### BACKGROUND OF THE INVENTION

I. Field of the Invention

The present invention relates to the fields of molecular biology and oncology. More specifically, the invention relates to methods and compositions involving microRNA molecules (miRNAs) for the treatment of cancer.

II. Background

Two models of tumor stem cell proliferation have been proposed. The stochastic model postulates that oncogenic mutations occur randomly in cells and that every cell within a tumor has a low but equal likelihood of re-initiating a tumor and thus functioning as a stem cell. In contrast, the cancer stem cell (CSC) model posits that tumors arise from a small, phenotypically distinct subset of cancer cells that cause the heterogeneous cell lineages observed in a tumor (Clarke et al., 2006).

CSCs have several properties that distinguish them from the remainder of the population. Most importantly, they undergo self-renewal—a unique type of cell division in which one or both progeny remains identical to the parent cell. In normal adult tissues, self-renewal is displayed exclusively by adult stem cells. Like adult stem cells, CSCs sit on top of the tumor cell hierarchy and can respond to stimuli to generate cells further along the differentiation spectrum, albeit in an aberrant manner (Clarke et al., 2006). CSCs are also resistant to chemotherapy and radiation therapy (Bao et al., 2006; Li et al., 2008; Hambardzumyan et al., 2008; Liu et al., 2006), which could explain why conventional treatments are ineffective in curing cancer and relapse occurs in generally more aggressive forms. Moreover, some CSCs are relatively quiescent shielding them from drugs that target highly proliferative cells (Holyoake et al., 1999; Guan and Hogge, 2000). Finally, CSCs can mediate metastasis in some cancers (Hermann et al., 2007; Patrawala et al., 2006).

Currently, cancer treatments are based on the stochastic model of tumor cell proliferation, targeting the bulk of the tumor without discrimination. Given that CSCs are the ‘root’ of the tumor and insensitive to most treatments, it is imperative that therapeutic modalities are developed to specifically eradicate them.

Recently, microRNAs have been implicated in various cancers. microRNAs (miRNAs) are short RNA molecules (16-29 nucleotides in length) that arise from longer precursors, which are transcribed from non-protein coding genes (Carrington et al., 2003). The precursors are processed by cellular proteins to generate short double-stranded miRNA. One of the miRNA strands is incorporated into a complex of proteins and miRNA called the RNA-induced silencing complex (RISC). The miRNA guides the RISC complex to a target mRNA, which is then cleaved or translationally silenced, depending on the degree of sequence complementarity of the miRNA or its target mRNA (Bagga et al., 2005; Lim et al., 2005).

Additional compositions and methods for identifying and treating these cancer stem cells is needed.

### SUMMARY OF THE INVENTION

The present invention includes compositions and methods related to identifying and treating cancer and/or cancer stem cells. In certain embodiments, compositions and methods are directed to miRNAs differentially expressed in cancer stem cells. In a further embodiment, the cancer stem cells can be associated with a variety of cancers, including but not limited to bone, brain, breast, colorectal, pancreatic, ovarian, prostate, testicular, hepatic, kidney, lung, or skin. In certain aspects, miRNAs are isolated from prostate tumors and represent targets for therapeutic intervention or diagnostic markers in prostate cancer. In a further aspect, hsa-miR-34a, hsa-miR-126 and let-7b are therapeutic targets or therapeutics that can inhibit cancer stem cell growth in vitro and in vivo.

The present invention provides additional compositions and methods for the identification and/or treatment of CSCs by identifying miRNAs that are differentially expressed or mis-regulated in CSCs. Further, the invention describes a method for treating cancer based on administering a molecule having an activity of selected miRNAs or miRNA inhibitors to a patient, tissue, or cells at risk of developing cancer, suspected of having cancer, or having cancer.

The term “miRNA” or “miR” is used according to its ordinary and plain meaning and refers to a microRNA molecule found in eukaryotes that is involved in RNA-based gene regulation. See, e.g., Carrington et al., 2003, which is hereby incorporated by reference. The term will be used to refer to the single-stranded RNA molecule processed from a precursor. Names of miRNAs and their sequences related to the present invention are provided herein.

miR sequences can be used to evaluate cells and/or tissue for the possibility of a condition associated with CSCs, particularly those conditions that will result in the development of a disease or a pathological condition associated with CSCs, namely cancer.

In certain aspects, an miRNA that is differentially expressed between a CSC and a non-CSC or normal cell is administered to a patient having or suspected of having precancerous or cancerous condition. In certain aspects, an inhibitor of a miRNA that is differentially expressed between CSC and a non-CSC or a normal cell is administered to a patient having, suspected of having, or at risk of cancer. In certain aspects the cancer is prostate cancer.

Embodiments of the invention include methods of modulating cellular nucleic acids and the processing of these nucleic acids comprising administering to the cell, tissue, or subject an amount of an isolated nucleic acid or mimetic thereof comprising all or part of an miRNA nucleic acid sequence, mimetic, or inhibitor sequence in an amount sufficient to modulate the processing of a cellular nucleic acid by positive or negative regulation, which includes one or more of regulating/modulating: transcription, mRNA levels, mRNA translation, and/or protein levels in a cell, tissue, or organ. In certain aspects the expression of a gene or level of a gene product, such as mRNA or encoded protein, is down-regulated or up-regulated. Administration of a nucleic acid or nucleic acid mimetic can result in a decrease in or cessation of growth, metabolism, or replication of a cancer stem cell. In certain aspects administration of a nucleic acid composition results in the death of some or all cancer stem cells.

In certain embodiments methods include treating cancer comprising administering to a subject in need of such therapy an effective amount of: (a) an inhibitor of hsa-let-7b, hsa-let-7c, hsa-let-7e, hsa-miR-100, hsa-miR-101, hsa-miR-105, hsa-miR-10a, hsa-miR-10b, hsa-miR-124a, hsa-miR-125a, hsa-miR-125b, hsa-miR-127, hsa-miR-133a, hsa-miR-133b, hsa-miR-140, hsa-miR-142-3p, hsa-miR-142-5p, hsa-miR-145, hsa-miR-146a, hsa-miR-146b, hsa-miR-147, hsa-miR-148a, hsa-miR-148b, hsa-miR-15b, hsa-miR-16, hsa-miR-181 a, hsa-miR-181d, hsa-miR-183, hsa-miR-18a, hsa-miR-190, hsa-miR-192, hsa-miR-193b, hsa-miR-195, hsa-miR-196a, hsa-miR-196b, hsa-miR-19a, hsa-miR-19b, hsa-miR-203, hsa-miR-205, hsa-miR-21, hsa-miR-214, hsa-miR-218, hsa-miR-22, hsa-miR-221, hsa-miR-222, hsa-miR-223, hsa-miR-24, hsa-miR-25, hsa-miR-26b, hsa-miR-27a, hsa-miR-27b, hsa-miR-28, hsa-miR-29a, hsa-miR-29b, hsa-miR-29c, hsa-miR-301, hsa-miR-30a-3p, hsa-miR-30a-5p, hsa-miR-30b, hsa-miR-30c, hsa-miR-30d, hsa-miR-30e-5p, hsa-miR-32, hsa-miR-320, hsa-miR-324-3p, hsa-miR-324-5p, hsa-miR-328, hsa-miR-33, hsa-miR-331, hsa-miR-338, hsa-miR-339, hsa-miR-345, hsa-miR-361, hsa-miR-374, hsa-miR-378, hsa-miR-422a, hsa-miR-422b, hsa-miR-423, hsa-miR-425, hsa-miR-433, hsa-miR-451, hsa-miR-452, hsa-miR-500, hsa-miR-501, hsa-miR-9, and/or hsa-miR-96; or (b) a molecule having hsa-let-7a, hsa-let-7b, hsa-let-7c, hsa-let-7e, hsa-let-7f, hsa-let-7i, hsa-miR-101, hsa-miR-103, hsa-miR-106a, hsa-miR-10a, hsa-miR-10b, hsa-miR-124a, hsa-miR-125a, hsa-miR-126, hsa-miR-132, hsa-miR-133a, hsa-miR-141, hsa-miR-146a, hsa-miR-146b, hsa-miR-148a, hsa-miR-148b, hsa-miR-149, hsa-miR-151, hsa-miR-152, hsa-miR-15a, hsa-miR-15b, hsa-miR-181b, hsa-miR-182, hsa-miR-183, hsa-miR-18a, hsa-miR-191, hsa-miR-193b, hsa-miR-195, hsa-miR-196a, hsa-miR-196b, hsa-miR-199a, hsa-miR-200a, hsa-miR-200c, hsa-miR-203, hsa-miR-205, hsa-miR-21, hsa-miR-214, hsa-miR-218, hsa-miR-22, hsa-miR-222, hsa-miR-24, hsa-miR-25, hsa-miR-26a, hsa-miR-27a, hsa-miR-29b, hsa-miR-29c, hsa-miR-30a-3p, hsa-miR-30a-5p, hsa-miR-30d, hsa-miR-320, hsa-miR-324-5p, hsa-miR-328, hsa-miR-331, hsa-miR-335, hsa-miR-340, hsa-miR-342, hsa-miR-345, hsa-miR-34a, hsa-miR-362, hsa-miR-365, hsa-miR-375, hsa-miR-378, hsa-miR-422a, hsa-miR-422b, hsa-miR-425, hsa-miR-429, hsa-miR-500, hsa-miR-7, hsa-miR-9*, hsa-miR-92, hsa-miR-93, hsa-miR-98, and/or hsa-miR-99b activity.

A “therapeutic nucleic acid sequence” includes nucleic acids that positively or negatively modulate the processing of a cellular nucleic acid. A therapeutic nucleic may include the full length or segments of precursor sequence of a miRNA identified herein, or a complement thereof. In certain aspects a therapeutic nucleic acid can comprise all or part of a processed (i.e., mature) miRNA sequence or complement thereof, including 5, 6, 7, 8, 9, 10, 11, 12, 13, 14, 15, 16, 17, 18, 19, 20, 21, 22, 23, 24, 25, 26, 27, 28, 29 or more nucleotides of an miRNA described herein. In other aspects a therapeutic nucleic acid can include all or part of a mature miRNA sequence or complement as well as 5, 6, 7, 8, 9, 10, 11, 12, 13, 14, 15, 16, 17, 18, 19, 20, 21, 22, 23, 24, 25, 26, 27, 28, 29 or more nucleotides of a precursor miRNA or its processed sequence, or complement thereof, including all ranges and integers there between. In certain embodiments, the therapeutic nucleic acid sequence contains a full-length processed miRNA sequence described herein or a complement thereof. In still further aspects, the therapeutic nucleic acid comprises about, at least, or at most a 5, 6, 7, 8, 9, 10, 11, 12, 13, 14, 15, 16, 17, 18, 19, 20, 21, 22, 23, 24, 25, 50 nucleotide segment (including all ranges and integers there between) or complementary segment of a miRNA described herein that is at least 75, 80, 85, 90, 95, 98, 99 or 100% identical to SEQ ID NO:1 to SEQ ID NO:119. In certain aspects, a subset of these miRNAs will be used that include some but not all of the miRNA described herein. In certain aspects, 1, 2, 3, 4, 5, 6, 7, 8, 9, 10, 11, 12, 13, 14, 15, 16, 17, 18, 19, 20 or more miRNAs or segments thereof (including complementary sequences) will be included with all other miRNA sequences excluded. In certain aspects, subsets of these miRNAs will be used that include some but not all of the listed miRNA sequences, segments, or complements.

In certain aspects the invention includes methods of treating a cancer stem cell or associated cancer comprising administering to the cell an amount of an isolated (a) nucleic acid inhibitor of hsa-let-7b, hsa-let-7c, hsa-let-7e, hsa-miR-100, hsa-miR-101, hsa-miR-105, hsa-miR-10a, hsa-miR-10b, hsa-miR-124a, hsa-miR-125a, hsa-miR-125b, hsa-miR-127, hsa-miR-133a, hsa-miR-133b, hsa-miR-140, hsa-miR-142-3p, hsa-miR-142-5p, hsa-miR-145, hsa-miR-146a, hsa-miR-146b, hsa-miR-147, hsa-miR-148a, hsa-miR-148b, hsa-miR-15b, hsa-miR-16, hsa-miR-181a, hsa-miR-181d, hsa-miR-183, hsa-miR-18a, hsa-miR-190, hsa-miR-192, hsa-miR-193b, hsa-miR-195, hsa-miR-196a, hsa-miR-196b, hsa-miR-19a, hsa-miR-19b, hsa-miR-203, hsa-miR-205, hsa-miR-21, hsa-miR-214, hsa-miR-218, hsa-miR-22, hsa-miR-221, hsa-miR-222, hsa-miR-223, hsa-miR-24, hsa-miR-25, hsa-miR-26b, hsa-miR-27a, hsa-miR-27b, hsa-miR-28, hsa-miR-29a, hsa-miR-29b, hsa-miR-29c, hsa-miR-301, hsa-miR-30a-3p, hsa-miR-30a-5p, hsa-miR-30b, hsa-miR-30c, hsa-miR-30d, hsa-miR-30e-5p, hsa-miR-32, hsa-miR-320, hsa-miR-324-3p, hsa-miR-324-5p, hsa-miR-328, hsa-miR-33, hsa-miR-331, hsa-miR-338, hsa-miR-339, hsa-miR-345, hsa-miR-361, hsa-miR-374, hsa-miR-378, hsa-miR-422a, hsa-miR-422b, hsa-miR-423, hsa-miR-425, hsa-miR-433, hsa-miR-451, hsa-miR-452, hsa-miR-500, hsa-miR-501, hsa-miR-9, and/or hsa-miR-96; or (b) nucleic acid having hsa-let-7a, hsa-let-7b, hsa-let-7c, hsa-let-7e, hsa-let-7f, hsa-let-7i, hsa-miR-101, hsa-miR-103, hsa-miR-106a, hsa-miR-10a, hsa-miR-10b, hsa-miR-124a, hsa-miR-125a, hsa-miR-126, hsa-miR-132, hsa-miR-133a, hsa-miR-141, hsa-miR-146a, hsa-miR-146b, hsa-miR-148a, hsa-miR-148b, hsa-miR-149, hsa-miR-151, hsa-miR-152, hsa-miR-15a, hsa-miR-15b, hsa-miR-181b, hsa-miR-182, hsa-miR-183, hsa-miR-18a, hsa-miR-191, hsa-miR-193b, hsa-miR-195, hsa-miR-196a, hsa-miR-196b, hsa-miR-199a, hsa-miR-200a, hsa-miR-200c, hsa-miR-203, hsa-miR-205, hsa-miR-21, hsa-miR-214, hsa-miR-218, hsa-miR-22, hsa-miR-222, hsa-miR-24, hsa-miR-25, hsa-miR-26a, hsa-miR-27a, hsa-miR-29b, hsa-miR-29c, hsa-miR-30a-3p, hsa-miR-30a-5p, hsa-miR-30d, hsa-miR-320, hsa-miR-324-5p, hsa-miR-328, hsa-miR-331, hsa-miR-335, hsa-miR-340, hsa-miR-342, hsa-miR-345, hsa-miR-34a, hsa-miR-362, hsa-miR-365, hsa-miR-375, hsa-miR-378, hsa-miR-422a, hsa-miR-422b, hsa-miR-425, hsa-miR-429, hsa-miR-500, hsa-miR-7, hsa-miR-9*, hsa-miR-92, hsa-miR-93, hsa-miR-98, and/or hsa-miR-99b activity; in an amount sufficient to treat cancer (an “*” indicates the miR is derived from the opposite arm of the precursor miRNA). Typically, a cell or subject can have, be suspected of having, or be at risk of developing cancer. In certain aspects the growth of a cancer stem cell is prevented, ameliorated, reduced, or eliminated.

In further aspects the invention includes methods of treating a cancer stem cell or associated cancer comprising administering to the cell an amount of 1, 2, 3, 4, 5, 6 or more of miR-34, let-7, miR-15, mir-16, miR106, miR-10, Mir-141, miR218, mir422a, and/or miR-335 in combination with an isolated (a) nucleic acid inhibitor of hsa-let-7b, hsa-let-7c, hsa-let-7e, hsa-miR-100, hsa-miR-101, hsa-miR-105, hsa-miR-10a, hsa-miR-10b, hsa-miR-124a, hsa-miR-125a, hsa-miR-125b, hsa-miR-127, hsa-miR-133a, hsa-miR-133b, hsa-miR-140, hsa-miR-142-3p, hsa-miR-142-5p, hsa-miR-145, hsa-miR-146a, hsa-miR-146b, hsa-miR-147, hsa-miR-148a, hsa-miR-148b, hsa-miR-15b, hsa-miR-16, hsa-miR-181 a, hsa-miR-181d, hsa-miR-183, hsa-miR-18a, hsa-miR-190, hsa-miR-192, hsa-miR-193b, hsa-miR-195, hsa-miR-196a, hsa-miR-196b, hsa-miR-19a, hsa-miR-19b, hsa-miR-203, hsa-miR-205, hsa-miR-21, hsa-miR-214, hsa-miR-218, hsa-miR-22, hsa-miR-221, hsa-miR-222, hsa-miR-223, hsa-miR-24, hsa-miR-25, hsa-miR-26b, hsa-miR-27a, hsa-miR-27b, hsa-miR-28, hsa-miR-29a, hsa-miR-29b, hsa-miR-29c, hsa-miR-301, hsa-miR-30a-3p, hsa-miR-30a-5p, hsa-miR-30b, hsa-miR-30c, hsa-miR-30d, hsa-miR-30e-5p, hsa-miR-32, hsa-miR-320, hsa-miR-324-3p, hsa-miR-324-5p, hsa-miR-328, hsa-miR-33, hsa-miR-331, hsa-miR-338, hsa-miR-339, hsa-miR-345, hsa-miR-361, hsa-miR-374, hsa-miR-378, hsa-miR-422a, hsa-miR-422b, hsa-miR-423, hsa-miR-425, hsa-miR-433, hsa-miR-451, hsa-miR-452, hsa-miR-500, hsa-miR-501, hsa-miR-9, and/or hsa-miR-96; or (b) nucleic acid having hsa-let-7a, hsa-let-7b, hsa-let-7c, hsa-let-7e, hsa-let-7f, hsa-let-7i, hsa-miR-101, hsa-miR-103, hsa-miR-106a, hsa-miR-10a, hsa-miR-10b, hsa-miR-124a, hsa-miR-125a, hsa-miR-126, hsa-miR-132, hsa-miR-133 a, hsa-miR-141, hsa-miR-146a, hsa-miR-146b, hsa-miR-148a, hsa-miR-148b, hsa-miR-149, hsa-miR-151, hsa-miR-152, hsa-miR-15a, hsa-miR-15b, hsa-miR-181b, hsa-miR-182, hsa-miR-183, hsa-miR-18a, hsa-miR-191, hsa-miR-193b, hsa-miR-195, hsa-miR-196a, hsa-miR-196b, hsa-miR-199a, hsa-miR-200a, hsa-miR-200c, hsa-miR-203, hsa-miR-205, hsa-miR-21, hsa-miR-214, hsa-miR-218, hsa-miR-22, hsa-miR-222, hsa-miR-24, hsa-miR-25, hsa-miR-26a, hsa-miR-27a, hsa-miR-29b, hsa-miR-29c, hsa-miR-30a-3p, hsa-miR-30a-5p, hsa-miR-30d, hsa-miR-320, hsa-miR-324-5p, hsa-miR-328, hsa-miR-331, hsa-miR-335, hsa-miR-340, hsa-miR-342, hsa-miR-345, hsa-miR-34a, hsa-miR-362, hsa-miR-365, hsa-miR-375, hsa-miR-378, hsa-miR-422a, hsa-miR-422b, hsa-miR-425, hsa-miR-429, hsa-miR-500, hsa-miR-7, hsa-miR-9*, hsa-miR-92, hsa-miR-93, hsa-miR-98, and/or hsa-miR-99b activity; in an amount sufficient to treat cancer (an “*” indicates the miR is derived from the opposite arm of the precursor miRNA). Typically, a cell or subject can have, be suspected of having, or be at risk of developing cancer. In certain aspects the growth of a cancer stem cell is prevented, ameliorated, reduced, or eliminated.

In further aspects the invention includes methods of treating a cancer stem cell or associated cancer comprising administering to the cell an amount of a nucleic acid comprising a sequence of miR-34 or an inhibitor thereof sufficient to treat cancer or eliminates, prevents, ameliorates, or reduces the growth of a cancer stem cell.

In still further aspects the invention includes methods of treating a cancer stem cell or associated cancer comprising administering to the cell an amount of a nucleic acid comprising a sequence of let-7 or an inhibitor thereof sufficient to treat cancer or eliminates, prevents, ameliorates, or reduces the growth of a cancer stem cell.

In certain aspects the invention includes methods of treating a cancer stem cell or associated cancer comprising administering to the cell an amount of a nucleic acid comprising a sequence of miR-15 or an inhibitor thereof sufficient to treat cancer or eliminates, prevents, ameliorates, or reduces the growth of a cancer stem cell.

In further aspects the invention includes methods of treating a cancer stem cell or associated cancer comprising administering to the cell an amount of a nucleic acid comprising a sequence of mir-16 or an inhibitor thereof sufficient to treat cancer or eliminates, prevents, ameliorates, or reduces the growth of a cancer stem cell.

In still further aspects the invention includes methods of treating a cancer stem cell or associated cancer comprising administering to the cell an amount of a nucleic acid comprising a sequence of miR-106 or an inhibitor thereof sufficient to treat cancer or eliminates, prevents, ameliorates, or reduces the growth of a cancer stem cell.

In certain aspects the invention includes methods of treating a cancer stem cell or associated cancer comprising administering to the cell an amount of a nucleic acid comprising a sequence of miR-10 or an inhibitor thereof sufficient to treat cancer or eliminates, prevents, ameliorates, or reduces the growth of a cancer stem cell.

In further aspects the invention includes methods of treating a cancer stem cell or associated cancer comprising administering to the cell an amount of a nucleic acid comprising a sequence of miR-141 or an inhibitor thereof sufficient to treat cancer or eliminates, prevents, ameliorates, or reduces the growth of a cancer stem cell.

In further aspects the invention includes methods of treating a cancer stem cell or associated cancer comprising administering to the cell an amount of a nucleic acid comprising a sequence of miR-218 or an inhibitor thereof sufficient to treat cancer or eliminates, prevents, ameliorates, or reduces the growth of a cancer stem cell.

In further aspects the invention includes methods of treating a cancer stem cell or associated cancer comprising administering to the cell an amount of a nucleic acid comprising a sequence of miR422a or an inhibitor thereof sufficient to treat cancer or eliminates, prevents, ameliorates, or reduces the growth of a cancer stem cell.

In further aspects the invention includes methods of treating a cancer stem cell or associated cancer comprising administering to the cell an amount of a nucleic acid comprising a sequence of miR-335 or an inhibitor thereof sufficient to treat cancer or eliminates, prevents, ameliorates, or reduces the growth of a cancer stem cell.

In further aspects the invention includes methods of treating a cancer stem cell or associated cancer comprising administering to the cell an amount of an inhibitor of hsa-miR-125b, hsa-miR-133b, hsa-miR-142-3p, hsa-miR-181a, hsa-miR-181d, hsa-miR-205, hsa-miR-221, hsa-miR-222, hsa-miR-223, hsa-miR-29a, hsa-miR-29c, hsa-miR-301, hsa-miR-328, hsa-miR-33, hsa-miR-338, hsa-miR-374, hsa-miR-378, hsa-miR-451, hsa-miR-452, and/or hsa-miR-501. The nucleic acid can be administered in an amount sufficient to treat cancer. Typically, a cell or subject can have, be suspected of having, or be at risk of developing cancer. In certain aspects the growth of a cancer stem cell is prevented, ameliorated, reduced, or eliminated.

In further aspects the invention includes methods of treating a cancer stem cell or associated cancer comprising administering to the cell an amount of an nucleic acid has an activity of one or more hsa-let-7c, hsa-miR-126, hsa-miR-15a, hsa-miR-15b, hsa-miR-182, hsa-miR-183, hsa-miR-191, hsa-miR-200a, hsa-miR-200c, hsa-miR-203, hsa-miR-218, hsa-miR-27a, hsa-miR-29b, hsa-miR-30a-3p, hsa-miR-30a-5p, hsa-miR-335, hsa-miR-340, hsa-miR-34a, hsa-miR-365, hsa-miR-375, hsa-miR-422a, hsa-miR-425, hsa-miR-92, and/or hsa-miR-93. The nucleic acid can be administered in an amount sufficient to treat cancer. Typically, a cell or subject can have, be suspected of having, or be at risk of developing cancer. In certain aspects the growth of a cancer stem cell is prevented, ameliorated, reduced, or eliminated.

In still further aspects, a nucleic acid has an activity of a hsa-let-7b, hsa-miR-34a, hsa-miR-126, hsa-miR-141, hsa-miR-335, hsa-miR-365, and/or hsa-miR-375.

In certain aspects a nucleic acid inhibitor is an inhibitor of hsa-miR-142-3p, hsa-miR-33, hsa-miR-301, hsa-miR-451, and/or hsa-miR-452.

In still a further aspect a hsa-miR-34a nucleic acid is administered.

In certain embodiments a therapeutic nucleic acid is a recombinant nucleic acid, such as RNA, DNA, or RNA/DNA hybrid. A recombinant nucleic acid of the invention can be comprised in a miRNA expression cassette, which can be further comprised in a nucleic acid vector such as a viral vector, or a plasmid vector. Typically a viral vector is administered at a dose of 1×10⁵ to 1×10¹⁴ viral particles per dose. A plasmid DNA vector is typically administered at a dose of 100 mg per patient to 4000 mg per patient.

In certain aspects the therapeutic nucleic acid is a synthetic nucleic acid. Typically a nucleic acid is administered at a dose of 0.01, 0.05, 0.1, 0.5, 1, 2, 3, 4, 5, 6 mg/kg of body weight to 5, 6, 7, 8, 9, 10 mg/kg of body weight, including all values and ranges there between. The nucleic acid can be administered enterally, parenterally, topically or by other administration methods well known to those skilled in the art. In certain aspects the nucleic acid is comprised in a pharmaceutical formulation, such as a lipid composition or a nanoparticle composition or biocompatible and biodegradable molecule composition.

In still a further aspect, 2, 3, 4, 5, 6, or more therapeutic nucleic acids may be administered together or sequentially. Typically if the nucleic acids are administered together, they will be in a single composition.

In yet still further aspects a therapy will reduce the viability of the cell, reduce proliferation of the cell, reduce metastasis of the cell, or increase the cell's sensitivity to therapy.

In yet another embodiment the invention includes methods of treating a patient diagnosed with or suspected of having or at risk of developing prostate cancer comprising the steps of: (a) administering to the patient an amount of an isolated therapeutic nucleic acid comprising a miRNA sequence in an amount sufficient to positively or negatively regulate a cellular pathway or treat a patient, wherein a therapeutic nucleic acid inhibitor of one or more of hsa-let-7b, hsa-let-7c, hsa-let-7e, hsa-miR-100, hsa-miR-101, hsa-miR-105, hsa-miR-10a, hsa-miR-10b, hsa-miR-124a, hsa-miR-125a, hsa-miR-125b, hsa-miR-127, hsa-miR-133a, hsa-miR-133b, hsa-miR-140, hsa-miR-142-3p, hsa-miR-142-5p, hsa-miR-145, hsa-miR-146a, hsa-miR-146b, hsa-miR-147, hsa-miR-148a, hsa-miR-148b, hsa-miR-15b, hsa-miR-16, hsa-miR-181 a, hsa-miR-181d, hsa-miR-183, hsa-miR-18a, hsa-miR-190, hsa-miR-192, hsa-miR-193b, hsa-miR-195, hsa-miR-196a, hsa-miR-196b, hsa-miR-19a, hsa-miR-19b, hsa-miR-203, hsa-miR-205, hsa-miR-21, hsa-miR-214, hsa-miR-218, hsa-miR-22, hsa-miR-221, hsa-miR-222, hsa-miR-223, hsa-miR-24, hsa-miR-25, hsa-miR-26b, hsa-miR-27a, hsa-miR-27b, hsa-miR-28, hsa-miR-29a, hsa-miR-29b, hsa-miR-29c, hsa-miR-301, hsa-miR-30a-3p, hsa-miR-30a-5p, hsa-miR-30b, hsa-miR-30c, hsa-miR-30d, hsa-miR-30e-5p, hsa-miR-32, hsa-miR-320, hsa-miR-324-3p, hsa-miR-324-5p, hsa-miR-328, hsa-miR-33, hsa-miR-331, hsa-miR-338, hsa-miR-339, hsa-miR-345, hsa-miR-361, hsa-miR-374, hsa-miR-378, hsa-miR-422a, hsa-miR-422b, hsa-miR-423, hsa-miR-425, hsa-miR-433, hsa-miR-451, hsa-miR-452, hsa-miR-500, hsa-miR-501, hsa-miR-9, and/or hsa-miR-96; and/or a therapeutic nucleic acid having an activity of one or more of hsa-let-7a, hsa-let-7b, hsa-let-7c, hsa-let-7e, hsa-let-7f, hsa-let-7i, hsa-miR-101, hsa-miR-103, hsa-miR-106a, hsa-miR-10a, hsa-miR-10b, hsa-miR-124a, hsa-miR-125a, hsa-miR-126, hsa-miR-132, hsa-miR-133a, hsa-miR-141, hsa-miR-146a, hsa-miR-146b, hsa-miR-148a, hsa-miR-148b, hsa-miR-149, hsa-miR-151, hsa-miR-152, hsa-miR-15a, hsa-miR-15b, hsa-miR-181b, hsa-miR-182, hsa-miR-183, hsa-miR-18a, hsa-miR-191, hsa-miR-193b, hsa-miR-195, hsa-miR-196a, hsa-miR-196b, hsa-miR-199a, hsa-miR-200a, hsa-miR-200c, hsa-miR-203, hsa-miR-205, hsa-miR-21, hsa-miR-214, hsa-miR-218, hsa-miR-22, hsa-miR-222, hsa-miR-24, hsa-miR-25, hsa-miR-26a, hsa-miR-27a, hsa-miR-29b, hsa-miR-29c, hsa-miR-30a-3p, hsa-miR-30a-5p, hsa-miR-30d, hsa-miR-320, hsa-miR-324-5p, hsa-miR-328, hsa-miR-331, hsa-miR-335, hsa-miR-340, hsa-miR-342, hsa-miR-345, hsa-miR-34a, hsa-miR-362, hsa-miR-365, hsa-miR-375, hsa-miR-378, hsa-miR-422a, hsa-miR-422b, hsa-miR-425, hsa-miR-429, hsa-miR-500, hsa-miR-7, hsa-miR-9*, hsa-miR-92, hsa-miR-93, hsa-miR-98, and/or hsa-miR-99b activity, and (b) administering a second therapy, wherein the regulation of the cellular pathway sensitizes the patient to a second therapy.

In certain aspects, components or genes of a cellular pathway, which may also be regulated by various combinations of miRNAs include, but are not limited to Nanog, Oct-3/4, Gli1, Smoothened, Patched-1, Wnt3A, β-catenin, Notch-1, Jagged-1, Sox2, CD133, CD44, Bmi-1, ABCG2, ABCB1, ITGA2, ITGB1, PTEN, Stat3, Sox4, ALDH1, Prostate stem cell antigen, integrin αV, NFKB1, Bcl-2, CXCR4, PAPPA, Tight Junction Protein 2, Abl-interactor-1, B-cell translocation gene 1, Interleukin-6, CASP8 and FADD-like apoptosis regulator, Smu-1, S100 calcium biding protein A3, Ras and EF-hand domain containing, Interferon gamma receptor 1, insulin growth factor-like family member 1, Microseminoprotein beta, CEACAM5, S100 calcium binding protein A7, Hydroxyprostaglandin dehydrogenase 15, CEACAM7, Trefoil factor 1, Prolactin-induced protein, Secretagogin, SWAP70, cyclin Y, C10orf9, cell division cycle 123 homolog, alkylation repair homolog 1, transducer of ERBB2, xenotropic and polytropic retrovirus receptor, CD59 molecule, low density lipoprotein-related protein 2, proteolipid protein 2, mitogen-activated protein kinase 14, matrix metallopeptidase 7, matrix Gla protein, myeloid leukemia factor 1, filaminB beta, sodium channel modifier 1, cornichon homolog 4, receptor accessory protein 5, solute carrier family 25 member 33, solute carrier family 44 member 1, transmembrane channel-like 4, zinc finger DHHC-type containing 2, toll-like receptor adaptor molecule 2, KDEL (SEQ ID NO:120) (Lys-Asp-Glu-Leu) endoplasmic reticulum protein retention receptor 3, glucosamine-6-phosphate deaminase 1, thioesterase superfamily member 2, debranching enzyme homolog 1, solute carrier family 38 member 9, N-acetyltrans-ferase 10, chromosome 16 open reading frame 33, lactate dehydrogenase A, major histocompatibility complex class I-related, leucyl-tRNA synthetase, GTP binding protein 1, protease serine 16, WAP four-disulfide core domain 2, aurora kinase B, 3-hydroxybutyrate dehydrogenase type 2, dehydrogenase/reductase (SDR family) member 4, abhydrolase domain containing 14B, EF-hand calcium binding domain 4A, enoyl coenzyme A hydratase domain containing 2, golgi autoantigen, ATPase type 13A3, prolyl endopeptidase-like, cytochrome P450, JTV1, isoprenyl-cysteine carboxyl methyltransferase, DNA (cytosine-5-)-methyltransferase 3 alpha, histamine N-methyltransferase, methyltransferase like 7A, methyltransferase like 2A, methyltransferase like 2B, ezrin, tumor protein D52, actin related protein ⅔ complex subunit 5 16 kDa, nucleolar protein 8, N-ehylmaleimide-sensitive factor, RAD23 homolog B, signal recognition particle 54 kDa, heat shock 70 kDa protein 2, mediator complex subunit 1, THAP domain containing, apoptosis associated protein 2, cold inducible RNA binding protein, small nuclear ribonucleoprotein polypeptide N, superkiller viralicidic activity 2-like 2, dual specificity phosphatase 10, signal sequence receptor, alpha, v-erb-a erythroblastic leukemia viral oncogene homolog 4, epithelial membrane protein 1, low density lipoprotein receptor-related protein associated protein 1, SCY1-like binding protein 1, CSTF1, Kelch-like 20, DNAJC13, amyloid beta (A4) precursor-like protein 2, sorbin and SH3 domain containing 2, DNAJB1, nebulette, SH3 domain binding glutamic acid-rich protein like, nudix, GABA(A) receptor-associated protein like 1, microtubule-associated protein tau, DCBLD1, serine threonine kinase, protein kinase N2, CSNK2A1, paired immunoglobin-like type 2 receptor beta, endoplasmic reticulum to nucleus signaling 1, serum/gluco-corticoid regulated kinase family, member 3, WEE1 homolog, microtubule associated serine/threonine kinase family member 4, chromosome 11 open reading frame, nucleoproin 37 kDa, CLTC, coatomer protein complex, subunit beta 2 (beta prime), solute carrier family 25, EGFR-coamplified and overexpressed protein, phosphodiesterase 8A, signal tranducing adaptor molecule, tubulin beta, sorting nexin 6, RAB23, phospholipase A2-activating protein, stanniocalcin 2, lactotransferrin, interferon regulator factor 9, ataxin 3, GTF3C3, glycogen synthase kinase 3 beta, kruppel-like factor 10, ELL2, zinc finger and BTB domain containing 20, iroquois homeobox 3, v-ets erythroblastosis virus E26 oncogene homolog 1, SERTA domain containing 1, transmembrane protein 101, v-maf musculo-aponeurotic fibrosarcoma oncogene homolog F, SFPQ, Cbp/p300-interacting transactivator, with Glu/Asp-rich carboxy-terminal domain 4, CCAAT/enhancer binding protein (C/EBP) delta, eukaryotic translation initiation factor 4E family member 2, heparan sulfate 2-O-sulfotransferase 1, alkylglycerone phosphate synthase, phospho-glycerate kinase 1, 5-aminoimidazole-4-carboxamide ribonucleotide formyltrans-ferase/IMP cyclohydrolase, ethanolamine kinase 1, asparagine-linked glycosylation 2 homolog, ubiquitin-conjugating enzyme E2F, membrane-associated ring finger, CCR4-NOT transcription complex, subunit, ring finger protein 8, PSMA5, AMMERCR1, integrator complex subunit 2, FAM152A, integrator complex subunit 8, THUMP domain containing 3, centrosomal protein 350 kDa, Ewing tumor-associated antigen 1, WD repeat domain 68, viral DNA polymerase-transactivated protein 6, chromosome 7 open reading frame 25, chromosome 2 open reading frame 60, chromosome 17 open reading frame 71, chromosome 7 open reading frame 36, elongation protein 4 homolog, nudE nuclear distribution gene E homolog, chromosome 1 open reading frame 63, FAM172A, FAM53C, immediate early response 5, KIAA0146, transmembrane protein 63A, NEDD4 binding protein 2-like 1, LY6/PLAUR domain containing 6B, BEX family member 5, KIAA1217, chromosome 4 open reading frame 7, chromosome 9 open reading frame 64, Zyg-11 homolog B, FAM160B1, limb region 1 homolog, nuclear casein kinase and cyclin-dependent kinase substrate 1, ankyrin repeat domain 13C, coiled-coil domain containing 43, UHRF1 binding protein 1, chromosome 1 open reading frame 163, AIDA, chromosome 4 open reading frame 32, nestin, c-kit, GLI2, MCL1, IL4, MUC1, ABCA3, SHH (sonic hedgehog), IHH (Indian hedgehog) or combinations thereof.

In still another aspect the invention includes methods of selecting a miRNA to be administered to a subject suspected of having, or having a propensity for developing cancer comprising: (a) determining an expression profile of 1, 2, 3, 4, 5, 6, 7, 8, 9, 10, 11, 12, 13, 14, 15, 16, 17, 18, 19, 20 or more miRNA selected from hsa-let-7a, hsa-let-7b, hsa-let-7c, hsa-let-7e, hsa-let-7f, hsa-let-7i, hsa-miR-100, hsa-miR-101, hsa-miR-103, hsa-miR-105, hsa-miR-106a, hsa-miR-10a, hsa-miR-10b, hsa-miR-124a, hsa-miR-125a, hsa-miR-125b, hsa-miR-126, hsa-miR-127, hsa-miR-132, hsa-miR-133a, hsa-miR-133b, hsa-miR-140, hsa-miR-141, hsa-miR-142-3p, hsa-miR-142-5p hsa-miR-145, hsa-miR-146a, hsa-miR-146b, hsa-miR-147, hsa-miR-148a, hsa-miR-148b, hsa-miR-149, hsa-miR-151, hsa-miR-152, hsa-miR-15a, hsa-miR-15b, hsa-miR-16, hsa-miR-181a, hsa-miR-181b, hsa-miR-181d, hsa-miR-182, hsa-miR-183, hsa-miR-18a, hsa-miR-190, hsa-miR-191, hsa-miR-192, hsa-miR-193b, hsa-miR-195, hsa-miR-196a, hsa-miR-196b, hsa-miR-199a, hsa-miR-19a, hsa-miR-19b, hsa-miR-200a, hsa-miR-200c, hsa-miR-203, hsa-miR-205, hsa-miR-21, hsa-miR-214, hsa-miR-218, hsa-miR-22, hsa-miR-221, hsa-miR-222, hsa-miR-223, hsa-miR-24, hsa-miR-25, hsa-miR-26a, hsa-miR-26b, hsa-miR-27a, hsa-miR-27b, hsa-miR-28, hsa-miR-29a, hsa-miR-29b, hsa-miR-29c, hsa-miR-301, hsa-miR-30a-3p, hsa-miR-30a-5p, hsa-miR-30b, hsa-miR-30c, hsa-miR-30d, hsa-miR-30e-5p, hsa-miR-32, hsa-miR-320, hsa-miR-324-3p, hsa-miR-324-5p, hsa-miR-328, hsa-miR-33, hsa-miR-331, hsa-miR-335, hsa-miR-338, hsa-miR-339, hsa-miR-340, hsa-miR-342, hsa-miR-345, hsa-miR-34a, hsa-miR-361, hsa-miR-362, hsa-miR-365, hsa-miR-374, hsa-miR-375, hsa-miR-378, hsa-miR-422a, hsa-miR-422b, hsa-miR-423, hsa-miR-425, hsa-miR-429, hsa-miR-433, hsa-miR-451, hsa-miR-452, hsa-miR-500, hsa-miR-501, hsa-miR-7, hsa-miR-9, hsa-miR-9*, hsa-miR-92, hsa-miR-93, hsa-miR-96, hsa-miR-98, and/or hsa-miR-99b; and (b) administering a second therapy, wherein the regulation of the cellular pathway sensitizes the patient to a second therapy; and (b) assessing the sensitivity of the subject to a therapeutic nucleic acid based on an expression profile; and (c) selecting one or more therapeutic nucleic acid for administration to the patient based on the assessed sensitivity. The method can further comprise treating the subject with 1, 2, 3, 4, 5, 6, 7, 8, 9, 10, or more therapeutic nucleic acids administered individually, or in one or more combinations.

Corresponding miRNA sequences that can be used in the context of the invention include, but are not limited to, all or a portion of those sequences in the sequence listing provided herein, as well as the miRNA precursor sequence, or complement of one or more of these miRNAs. Any one or subset of these sequences can be specifically excluded from the claimed invention.

In some embodiments, it may be useful to know whether a cell expresses a particular miRNA endogenously or whether such expression is affected under particular conditions or when it is in a particular disease state, e.g., cancer. Thus, in some embodiments of the invention, methods include assaying a cell or a sample containing a cell for the presence of one or more miRNA. In certain aspects, the miRNAs evaluated include those differentially expressed when the cell is a CSC. Consequently, in some embodiments, methods include a step of generating a miRNA profile for a sample. The term “miRNA profile” refers to data regarding the expression pattern of miRNAs in the sample (e.g., one or more miRNA from Table 1). It is contemplated that the miRNA profile can be obtained using a set of miRNAs, using for example nucleic acid amplification or hybridization techniques well know to one of ordinary skill in the art. In certain embodiments, expression of one or more miRNA of SEQ ID NO:1 to SEQ ID NO:119 is evaluated prior to or after administration of a therapeutic miRNA.

In some embodiments of the invention, an miRNA profile is generated by steps that include one or more of: (a) labeling miRNA in the sample; (b) hybridizing miRNA to a number of probes, or amplifying a number of miRNA, and/or (c) determining miRNA hybridization to the probes or detecting miRNA amplification products, wherein a miRNA expression is evaluated. See U.S. patent application Ser. Nos. 11/141,707 and 11/855,792, all of which are hereby incorporated by reference.

It is specifically contemplated that miRNA profiles for patients, particularly those suspected of having a particular disease or condition, such as cancer, can be generated by evaluating one or more miRNA or set of miRNAs discussed in this application. The miRNA profile that is generated from the patient will be one that provides information regarding the particular disease, condition, or therapeutic target. In certain aspects, a party evaluating miRNA expression may prepare a recommendation, report and/or summary conveying processed or raw data to a diagnosing physician. In certain aspects, a miRNA profile can be used in conjunction with other diagnostic tests or therapies.

Still a further embodiment includes methods of treating a patient with a pathological condition related to the presence of CSCs comprising one or more of step of (a) administering to the patient an amount of a therapeutic nucleic acid comprising all or part of a miRNA nucleic acid sequence or complement thereof in an amount sufficient to modulate the expression of one or more genes, mRNA, and/or protein expression; and, alternatively, (b) administering a second therapy, wherein the modulation of one or more genes, mRNA, and/or protein sensitizes the patient to the second therapy. A second therapy can include administration of a second therapeutic nucleic acid, or may include various standard therapies, chemotherapy, radiation therapy, drug therapy, immunotherapy, surgery, and the like.

Embodiments of the invention may also include the determination or assessment of a gene expression or miRNA profile for the selection of an appropriate therapy. A physician may choose to treat a cancer using therapeutic nucleic acids of the invention in combination with standard treatment such as chemotherapy, radiation therapy, and/or surgery (e.g., freezing with liquid nitrogen (cryotherapy), electrocautery, surgical excision, laser treatments) and/or other methods.

Certain aspects of the invention include methods of treating a subject with a condition related to cancer comprising one or more of the steps of (a) determining an expression profile of one or more miRNA selected from those miRNA described herein (b) assessing the sensitivity or amenability of the subject to therapy based on the expression profile; (c) selecting a therapy based on the assessment of the patient in light of the miRNA profile; and (d) treating the subject using one or more selected therapy.

Because “cancer” refers to a class of diseases, it is unlikely that there will be a single treatment. Aspects of the invention can be used to determine which treatment will be most effective or most harmful, and provide a guide for the physician in evaluating, assessing, and formulating a treatment strategy for a patient.

Therapeutic nucleic acids may also include various heterologous nucleic acid sequences, i.e., those sequences not typically found operatively coupled with miRNA in nature, such as promoters, enhancers, and the like. The therapeutic nucleic acid can be a recombinant nucleic acid, and can be a ribonucleic acid and/or a deoxyribonucleic acid. The recombinant nucleic acid may comprise a therapeutic nucleic acid expression cassette, i.e., a nucleic acid segment that expresses a therapeutic nucleic acid when introduce into an environment containing components for nucleic acid synthesis. In a further aspect, the expression cassette is comprised in a viral vector, or plasmid DNA vector or other therapeutic nucleic acid vector or delivery vehicle, including liposomes and the like. In certain aspects, viral vectors can be administered at 1×10², 1×10³, 1×10⁴ 1×10⁵, 1×10⁶, 1×10⁷, 1×10⁸, 1×10⁹, 1×10¹⁰, 1×10¹¹, 1×10¹², 1×10¹³, 1×10¹⁴ pfu or viral particle (vp). In still further aspects, a therapeutic nucleic acid or a DNA comprising a therapeutic nucleic acid of the invention can be administered at 0.001, 0.01, 0.1, 1, 10, 20, 30, 40, 50, 100, 200, 400, 600, 800, 1000, 2000, to 4000 ng, μg, or mg, including all values and ranges there between.

In certain aspects, the therapeutic nucleic acid is a synthetic nucleic acid. Moreover, nucleic acids of the invention may be fully or partially synthetic. In yet a further aspect, nucleic acids of the invention, including synthetic nucleic acid, can be administered at 0.001, 0.01, 0.1, 1, 10, 20, 30, 40, 50, 100, to 200 ng, μg, or mg per kilogram (kg) of body weight. Each of the amounts described herein may be administered over a period of time, including 0.5, 1, 2, 3, 4, 5, 6, 7, 8, 9, 10, minutes, hours, days, weeks, months or years, including all values and ranges there between.

In certain embodiments, administration of the composition(s) can be enteral or parenteral. In certain aspects, enteral administration is oral. In further aspects, parenteral administration is intralesional, intravascular, intracranial, intrapleural, intratumoral, intraperitoneal, intramuscular, intralymphatic, intraglandular, subcutaneous, topical, intrabronchial, intratracheal, intranasal, inhaled, or instilled. Compositions of the invention may be administered regionally or locally and not necessarily directly into a lesion.

A further embodiment of the invention includes methods of detecting cancer or CSCs in a biological sample comprising evaluating expression levels of 1, 2, 3, 4, 5, 6, 7, 8 9, 10, 11, 12, 13, 14, 15, 16, 17, 18, 19, 20, or more of hsa-let-7a, hsa-let-7b, hsa-let-7c, hsa-let-7e, hsa-let-7f, hsa-let-7i, hsa-miR-100, hsa-miR-101, hsa-miR-103, hsa-miR-105, hsa-miR-106a, hsa-miR-10a, hsa-miR-10b, hsa-miR-124a, hsa-miR-125a, hsa-miR-125b, hsa-miR-126, hsa-miR-127, hsa-miR-132, hsa-miR-133a, hsa-miR-133b, hsa-miR-140, hsa-miR-141, hsa-miR-142-3p, hsa-miR-142-5p, hsa-miR-145, hsa-miR-146a, hsa-miR-146b, hsa-miR-147, hsa-miR-148a, hsa-miR-148b, hsa-miR-149, hsa-miR-151, hsa-miR-152, hsa-miR-15a, hsa-miR-15b, hsa-miR-16, hsa-miR-181a, hsa-miR-181b, hsa-miR-181d, hsa-miR-182, hsa-miR-183, hsa-miR-18a, hsa-miR-190, hsa-miR-191, hsa-miR-192, hsa-miR-193b, hsa-miR-195, hsa-miR-196a, hsa-miR-196b, hsa-miR-199a, hsa-miR-19a, hsa-miR-19b, hsa-miR-200a, hsa-miR-200c, hsa-miR-203, hsa-miR-205, hsa-miR-21, hsa-miR-214, hsa-miR-218, hsa-miR-22, hsa-miR-221, hsa-miR-222, hsa-miR-223, hsa-miR-24, hsa-miR-25, hsa-miR-26a, hsa-miR-26b, hsa-miR-27a, hsa-miR-27b, hsa-miR-28, hsa-miR-29a, hsa-miR-29b, hsa-miR-29c, hsa-miR-301, hsa-miR-30a-3p, hsa-miR-30a-5p, hsa-miR-30b, hsa-miR-30c, hsa-miR-30d, hsa-miR-30e-5p, hsa-miR-32, hsa-miR-320, hsa-miR-324-3p, hsa-miR-324-5p, hsa-miR-328, hsa-miR-33, hsa-miR-331, hsa-miR-335, hsa-miR-338, hsa-miR-339, hsa-miR-340, hsa-miR-342, hsa-miR-345, hsa-miR-34a, hsa-miR-361, hsa-miR-362, hsa-miR-365, hsa-miR-374, hsa-miR-375, hsa-miR-378, hsa-miR-422a, hsa-miR-422b, hsa-miR-423, hsa-miR-425, hsa-miR-429, hsa-miR-433, hsa-miR-451, hsa-miR-452, hsa-miR-500, hsa-miR-501, hsa-miR-7, hsa-miR-9, hsa-miR-9*, hsa-miR-92, hsa-miR-93, hsa-miR-96, hsa-miR-98, hsa-miR-99b or combinations thereof.

An increase in the level of hsa-let-7b, hsa-let-7c, hsa-let-7e, hsa-miR-100, hsa-miR-101, hsa-miR-105, hsa-miR-10a, hsa-miR-10b, hsa-miR-124a, hsa-miR-125a, hsa-miR-125b, hsa-miR-127, hsa-miR-133a, hsa-miR-133b, hsa-miR-140, hsa-miR-142-3p, hsa-miR-142-5p, hsa-miR-145, hsa-miR-146a, hsa-miR-146b, hsa-miR-147, hsa-miR-148a, hsa-miR-148b, hsa-miR-15b, hsa-miR-16, hsa-miR-181a, hsa-miR-181d, hsa-miR-183, hsa-miR-18a, hsa-miR-190, hsa-miR-192, hsa-miR-193b, hsa-miR-195, hsa-miR-196a, hsa-miR-196b, hsa-miR-19a, hsa-miR-19b, hsa-miR-203, hsa-miR-205, hsa-miR-21, hsa-miR-214, hsa-miR-218, hsa-miR-22, hsa-miR-221, hsa-miR-222, hsa-miR-223, hsa-miR-24, hsa-miR-25, hsa-miR-26b, hsa-miR-27a, hsa-miR-27b, hsa-miR-28, hsa-miR-29a, hsa-miR-29b, hsa-miR-29c, hsa-miR-301, hsa-miR-30a-3p, hsa-miR-30a-5p, hsa-miR-30b, hsa-miR-30c, hsa-miR-30d, hsa-miR-30e-5p, hsa-miR-32, hsa-miR-320, hsa-miR-324-3p, hsa-miR-324-5p, hsa-miR-328, hsa-miR-33, hsa-miR-331, hsa-miR-338, hsa-miR-339, hsa-miR-345, hsa-miR-361, hsa-miR-374, hsa-miR-378, hsa-miR-422a, hsa-miR-422b, hsa-miR-423, hsa-miR-425, hsa-miR-433, hsa-miR-451, hsa-miR-452, hsa-miR-500, hsa-miR-501, hsa-miR-9, and/or hsa-miR-96 expression is indicative of cancer or CSCs. A decrease in the level of hsa-let-7a, hsa-let-7b, hsa-let-7c, hsa-let-7e, hsa-let-7f, hsa-let-7i, hsa-miR-101, hsa-miR-103, hsa-miR-106a, hsa-miR-10a, hsa-miR-10b, hsa-miR-124a, hsa-miR-125a, hsa-miR-126, hsa-miR-132, hsa-miR-133a, hsa-miR-141, hsa-miR-146a, hsa-miR-146b, hsa-miR-148a, hsa-miR-148b, hsa-miR-149, hsa-miR-151, hsa-miR-152, hsa-miR-15a, hsa-miR-15b, hsa-miR-181b, hsa-miR-182, hsa-miR-183, hsa-miR-18a, hsa-miR-191, hsa-miR-193b, hsa-miR-195, hsa-miR-196a, hsa-miR-196b, hsa-miR-199a, hsa-miR-200a, hsa-miR-200c, hsa-miR-203, hsa-miR-205, hsa-miR-21, hsa-miR-214, hsa-miR-218, hsa-miR-22, hsa-miR-222, hsa-miR-24, hsa-miR-25, hsa-miR-26a, hsa-miR-27a, hsa-miR-29b, hsa-miR-29c, hsa-miR-30a-3p, hsa-miR-30a-5p, hsa-miR-30d, hsa-miR-320, hsa-miR-324-5p, hsa-miR-328, hsa-miR-331, hsa-miR-335, hsa-miR-340, hsa-miR-342, hsa-miR-345, hsa-miR-34a, hsa-miR-362, hsa-miR-365, hsa-miR-375, hsa-miR-378, hsa-miR-422a, hsa-miR-422b, hsa-miR-425, hsa-miR-429, hsa-miR-500, hsa-miR-7, hsa-miR-9*, hsa-miR-92, hsa-miR-93, hsa-miR-98, and/or hsa-miR-99b expression is indicative of cancer or CSCs.

The present invention also concerns kits containing compositions of the invention or compositions to implement methods of the invention. In some embodiments, kits can be used to administer one or more therapeutic nucleic acid molecules. In certain embodiments, a kit contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more therapeutic nucleic acids related to miRNAs described herein and include all or part of an miRNA and/or synthetic miRNA molecules and/or miRNA inhibitors, or any range and combination derivable therein. In some embodiments, there are kits for evaluating miRNA activity in a cell and assessing the effectiveness of nucleic acid therapy.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using therapeutic nucleic acids and/or miRNA inhibitors of the invention for therapeutic applications are included as part of the invention. Specifically contemplated are any such molecules corresponding to any miRNA effective against cancer or CSCs, such as those discussed herein.

It is contemplated that any method or composition described herein can be implemented with respect to any other method or composition described herein and that different embodiments may be combined. It is specifically contemplated that any methods and compositions discussed herein with respect to therapeutic nucleic acids, miRNA molecules, and/or miRNA inhibitors may be implemented with respect to synthetic miRNAs. Nucleic acids may be exposed to conditions that allow it to become processed under physiological circumstances. The claims originally filed are contemplated to cover claims that are multiply dependent on any filed claim or combination of filed claims.

Any embodiment of the invention involving specific miRNAs by name is contemplated also to cover embodiments involving miRNAs whose sequences are at least 80, 81, 82, 83, 84, 85, 86, 87, 88, 89, 90, 91, 92, 93, 94, 95, 96, 97, 98, 99% identical to the mature sequence or precursor sequence of a specified miRNA(s). In other aspects miRNA of the invention may include additional nucleotides at the 5′,3′, or both 5′ and 3′ ends of at least, at most or about 1, 2, 3, 4, 5, 6, 7, 8, 9, 10 or more nucleotides.

Embodiments of the invention include kits for analysis of a pathological sample by assessing miRNA profile for a sample comprising, in suitable container means, two or more miRNA probes and/or amplification primers, wherein the miRNA probes detect or primer amplify one or more miRNA described herein. The kit can further comprise reagents for labeling miRNA in the sample. The kit may also include the labeling reagents include at least one amine-modified nucleotide, poly(A) polymerase, and poly(A) polymerase buffer. Labeling reagents can include an amine-reactive dye.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” may be used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates differential expression of miRNAs in CD44⁺ CSCs derived from freshly isolated human prostate tumor samples. Expression of miRNAs in CSCs was normalized to the expression of miRNAs in the CD44⁻ cancer cells (100%). Each vertical bar represents miRNA expression in a single human prostate tumor sample.

FIG. 2 illustrates effects of hsa-miR-34a on tumor growth of prostate cancer stem cells. Purified CD44+ Du145 prostate cancer cells were infected with lentivirus carrying hsa-miR-34a (miR-34a) or negative control miRNA (ctl). Each 10⁴ cells were injected into the dorsal prostate of NOD/SCID mice. Mice injected with miR-34 treated LAPC9 cells did not develop tumors.

FIG. 3 illustrates LAPC9 prostate cancer tumors from eight mice, forty-eight days following transfection of anti-miR-34a or negative control miRNA (anti-miR-NC) into LAPC9 cells and subsequent injection of 10⁵ cells into the dorsal prostate of NOD/SCID mice.

FIG. 4 illustrates long-term effects of hsa-miR-34a on cultured human PPC-1, PC3 and Du145 prostate cancer cells. Equal numbers cells were electroporated with 1.6 μM hsa-miR-34a (white squares) or negative control miRNA (NC, black diamonds), seeded and propagated in regular growth medium. When the control cells reached confluence (days 4 and 11 for PPC-1, days 7 and 14 for PC3 and Du145), cells were harvested, counted and electroporated again with the respective miRNAs. The population doubling and cumulative cell counts was calculated and plotted on a linear scale. Arrows represent electroporation days. Experiments with PC3 and Du145 cells were carried out in triplicates. Standard deviations are shown in the graphs. Abbreviation: miR-34a, hsa-miR-34a; NC, negative control miRNA.

FIG. 5 illustrates the average tumor volumes in groups of seven (n=7) mice carrying human PPC-1 prostate cancer xenografts. Human PPC-1 prostate tumor cells were treated with hsa-miR-34a (white squares) or with a negative control miRNA (NC, black diamonds) on days 0, 7, 13, 20, and 25 (arrows). Tumor growth was determined by caliper measurements for 32 days. Standard deviations are shown in the graph. All data points yielded p values<0.01. The p value obtained from data on day 22 is indicated by a circle. Abbreviation: miR-34a, hsa-miR-34a; NC, negative control miRNA.

FIG. 6 illustrates the histology of tumors that developed from PPC-1 prostate cancer cells treated with negative control miRNA (right) or hsa-miR-34a (left). Images show tumors stained with hematoxylin and eosin. The arrow indicates a pocket with seemingly viable cells. Abbreviation: miR-34a, hsa-miR-34a; NC, negative control miRNA.

FIG. 7 illustrates immunohistochemistry of PPC-1 tumors treated with negative control miRNA (top panels) or hsa-miR-34a (bottom panels). For hsa-miR-34a-treated tumors, the analysis is limited to areas with seemingly viable cells as shown in FIG. 8. Left images show tumor cells stained with hematoxylin and eosin (H&E); center images show an immunohistochemistry analysis using antibodies against the Ki-67 antigen (dark spotted areas); right images show an immunohistochemistry analysis using antibodies against caspase 3. Areas with increased apoptotic activity are exemplarily denoted by arrows. Abbreviation: miR-34a, hsa-miR-34a; NC, negative control miRNA.

FIG. 8 Survival curve. LAPC9-GFP cells (500,000) were orthotopically implanted in the dorsal prostate of male NOD/SCID mice. Twenty-one days later, animals were divided into four groups (n=7 for each group) and injected, via tail vein, with PBS (diamonds); vehicle control, (circles); miR-NC (triangles); and hsa-miR-34a (squares) on day 0. Injections were repeated on days 2, 4, 6, 8, and 10. Survival was tracked and plotted for 13 days.

FIG. 9 illustrates the long-term effects of hsa-miR-34a on cultured human H226 lung cancer cell numbers. Equal numbers of H226 cells were electroporated with 1.6 μM hsa-miR-34a (white squares) or negative control miRNA (NC, black diamonds), seeded and propagated in regular growth medium. When the control cells reached confluence (days 6, 17 and 25), cells were harvested, counted and electroporated again with the respective miRNAs. The population doubling and cumulative cell counts was calculated and plotted on a linear scale. Arrows represent electroporation days. Abbreviation: miR-34a, hsa-miR-34a; NC, negative control miRNA.

FIG. 10 illustrates average tumor volumes in groups of six (n=6) mice carrying human H460 lung cancer xenografts. Palpable tumors were treated with hsa-miR-34a (white squares) or with a negative control miRNA (NC, black diamonds) on days 11, 14, and 17 (arrows). Standard deviations are shown in the graph. Data points with p values<0.1, <0.05 and <0.01 are indicated by a cross, an asterisk or circles, respectively. Abbreviation: miR-34a, hsa-miR-34a; NC, negative control miRNA.

FIG. 11 illustrates the average tumor volumes in groups of seven (n=7) mice carrying human PPC-1 prostate cancer xenografts. Human PPC-1 prostate tumor cells were treated with hsa-miR-126 (white squares) or with a negative control miRNA (NC, black diamonds) on days 0, 7, 13 and 20 (arrows). Tumor growth was determined by caliper measurements for 22 days. Standard deviations are shown in the graph. Data points with p values<0.01 are indicated by a circle. Abbreviation: miR-126, hsa-miR-126; NC, negative control miRNA.

FIG. 12 illustrates average tumor volumes in groups of six (n=6) mice carrying human H460 lung cancer xenografts. Palpable tumors were treated with hsa-miR-126 (white squares) or with a negative control miRNA (NC, black diamonds) on days 11, 14, and 17 (arrows). Standard deviations are shown in the graph. Data points with p values<0.05 and <0.01 are indicated by an asterisk or circles, respectively. Abbreviation: miR-126, hsa-miR-126; NC, negative control miRNA.

FIG. 13 illustrates average tumor volumes in groups of six (n=6) mice carrying human H460 lung cancer xenografts. Palpable tumors were treated with hsa-let-7b (white squares) or with a negative control miRNA (NC, black diamonds) on days 11, 14, and 17 (arrows). Standard deviations are shown in the graph. Data points with p values<0.01 are indicated circles. Abbreviation: let-7b, hsa-let-7b; NC, negative control miRNA.

### DETAILED DESCRIPTION OF THE INVENTION

The present invention is directed to compositions and methods relating to preparation and characterization of miRNAs, as well as use of miRNAs for therapeutic, prognostic, and diagnostic applications, particularly those methods and compositions related to assessing and/or identifying cancer, CSCs and related conditions or diseases. In certain aspects the cancer is prostate cancer. In certain aspects the cancer is lung cancer. Other conditions amenable to the methods described herein include benign prostatic hyperplasia (BPH).

### I. Identification and Isolation of Stem Cells and CSCs

The most common method of identifying CSCs in a tumor is through the identification of cell surface markers that also identify normal stem cells in the tissue of origin. For instance, leukemic stem cells (LSCs) express the CD34 surface marker and lack the CD38 surface antigen, as is the case for normal (i.e., non-leukemic) hematopoietic stem cells (Bonnet and Dick, 1997). Prostate CSCs are positive for the CD133 and CD44 surface antigens (CD133⁺CD44⁺) and thus exhibit the same CD133⁺/CD44⁺ phenotype as normal prostate stem cells (Collins et al., 2005; Richardson et al., 2004). CSCs identified by cell surface marker expression can be purified by methods such as fluorescence-activated cell sorting (FACS).

Besides cell surface marker expression, functional assays are also frequently used to identify and isolate CSCs. Normal stem cells and CSCs preferentially express ATP-binding cassette (ABC) transporters, membrane protein complexes that enable the cells to resist cytotoxic drugs by actively transporting toxic compounds out of the cell. When cells are stained with the Hoechst 33342 fluorescent dye, ABC transporters in the stem cells actively pump the dye out of the cell. Stem cells thus appear as a “Hoechst-low” population when analyzed by FACS; whereas non-stem cells are more highly fluorescent. Since the “Hoechst-low” fraction lies on the side of the FACS profile, it is termed the ‘Side Population’ (SP). The SP selection technique has been successfully applied to identify and isolate stem cells and CSCs from a variety of tissues, including the blood, brain, and breast (Zhou et al., 2001; Goodell et al., 1996; Hirschmann-Jax et al., 2004; Patrawala et al., 2005; Engelmann et al., 2008).

Using well-established xenograft models, Patrawala et al. (2007) determined that prostate cancer cells are organized as a hierarchy encompassing a spectrum of cells at different stages of differentiation (Patrawala et al., 2007). In LAPC-9, a prostate xenograft isolated from a bone metastasis (Reiters and Sawyers, 2001), the SP cells comprise 0.1% of the total population and are 1000-fold more tumorigenic than the corresponding non-SP cells (Patrawala et al., 2005). CD44, a marker for normal prostate stem cells, identifies CSCs in LAPC-9, LAPC-4 (lymph node metastasis; Reiters and Sawyers, 2001) and Du145 (brain metastasis; Stone et al., 1978) tumors. CD44⁺ cells are more tumorigenic, invasive, and metastatic, as compared with their more differentiated counterparts, and display asymmetric cell-division, a hall-mark of self-renewal (Patrawala et al., 2006).

Both, the SP and CD44-expressing cells preferentially express genes that are associated with stem cell capacity, such as genes in the Notch, Wnt, and Hedgehog (Hh) signaling pathways. These three pathways are frequently implicated in self-renewal and maintenance of an undifferentiated state in stem cells and CSCs. For example, Hh signaling is required for prostate epithelium generation, and constitutive Hh signaling in undifferentiated stem/progenitor cells leads to oncogenic transformation (Karhadkar et al., 2004). Knockdown of Gli2, the transcription factor that primarily mediates Hh signaling, results in inhibition of colony-formation and tumor growth of prostate cancer cells (Thiyagarajan et al., 2007). Hh pathway components PTCH, Gli1, and Gli2 also regulate self-renewal of CD44⁺CD24⁻ breast CSCs (Liu et al., 2006). Treatment of medulloblastoma cells with cyclopamine, a Hh pathway antagonist, results in induction of genes related to neuronal differentiation (Berman et al., 2002). Notch and Wnt/β-catenin signaling are also required for maintenance of CSCs in a number of tumors (Malanchi et al., 2008; Li et al., 2003). Other stem cell capacity genes misregulated in CSCs include ALDH (Ginestier et al., 2007; Dylla et al., 2008), embryonic stem cell transcription factors such as Nanog, Oct-4, Sox-2, and Sox-4 (Zhang et al., 2008; Gu et al., 2007), PTEN (Hambardzumyan et al., 2008; Wang et al., 2006), and Bmi (Li et al., 2003; Hurt et al., 2008). These observations highlight the value of this stem cell-related pathway for treating cancers.

Several groups have also published large-scale gene-expression analyses comparing CSCs to their non-CSC counterparts (Hurt et al., 2008; Birnie et al., 2008; Liu et al., 2007). Liu et al. (2007) showed that the ‘invasive gene signature’ (IGS) derived from CD44⁺CD24⁻ breast CSCs could predict overall survival of patients with breast cancer, medulloblastoma, lung cancer, and prostate cancer. An independent study investigating gene expression in prostate CSCs found that 118 of 187 IGS genes were also represented in their arrays (Hurt et al., 2008). These data reiterate the notion that common pathways govern CSC properties across a variety of cancers.

Common genetic regulatory mechanisms govern the unique properties of CSCs from different tumor types. For instance, cross-talk between hedgehog signaling components PTCH and Gli1, and the Polycomb protein Bmi-1 regulates self-renewal in CD44⁺CD24⁻ breast CSCs (Bao et al., 2006). Hedgehog signaling maintains self-renewal in normal prostate stem cells, and aberrant activation of this pathway (mediated through Bmi-1 and SMO) leads to neoplastic transformation of the cells (Karhadkar et al., 2004; Fan et al., 2004). Other CSCs requiring Sonic Hedgehog activation for survival include those from multiple myeloma (Peacock et al., 2007), leukemia (Lessard and Sauvageau, 2003), and pancreatic cancer (Li et al., 2007). In short, CSCs derived from various cancers appear to have shared characteristics that are driven by the same mechanistic forces.

Other genes frequently implicated in CSC maintenance include PTEN (Wang et al., 2006; Hambardzumyan et al., 2008), Stat3 (Zhou et al., 2007), Bcl-2 (Konopleva et al., 2006), Ras (Yu et al., 2007), TGF-β (Shipitsin et al., 2007; Tang et al., 2007), and genes in the Notch (Fan et al., 2006; Shipitsin et al., 2007) and Wnt (Li et al., 2003; Jamieson et al., 2004) pathways. Furthermore, embryonic stem cell genes such as Nanog, Oct-4, Sox-2, and Sox-4 are increasingly reported to be associated with CSC self-renewal (Zhang et al., 2008; Gu et al., 2007; Zhang et al., 2006; Hurt et al., 2008). Several investigators have compared gene expression in CSCs with gene expression in their non-CSC counterparts or in normal stem cells from the matched tissue to generate CSC gene expression signatures for individual tumor types (Shepherd et al., 2008; Birnie et al., 2008; Liu et al., 2007; Shipitsin et al., 2007; Liu et al., 2006; Beier et al., 2007). In addition, expression patterns of genes in breast CSCs, also known as the ‘Invasive Gene Signature’ (IGS), were successfully used to predict the clinical outcome of patients that developed not only metastatic breast cancer, but also prostate cancer, lung cancer, and medulloblastoma (Liu et al., 2007), highlighting the prognostic value of this information.

### II. Therapeutic Methods

Certain embodiments of the invention concern nucleic acids that perform the activities of or inhibit endogenous miRNAs, mRNA, or other cellular components when introduced into cells. In certain aspects, therapeutic nucleic acids (also referred to as nucleic acids) can be synthetic, non-synthetic, or a combination of synthetic and non-synthetic miRNA sequences. Sequence-specific miRNA inhibitors can be used to inhibit sequentially or in combination the activities of one or more miRNAs in cells, as well those genes and associated pathways (including those pathways indicative of CSCs) modulated by an miRNA.

The present invention concerns, in certain aspects, short nucleic acid molecules (therapeutic nucleic acids) that function as miRNAs or as inhibitors of miRNA in a cell. The term “short” refers to a length of a single polynucleotide that is 10, 15, 16, 17, 18, 19, 20, 21, 22, 23, 24, 25, 50, 100, or 150 nucleotides or fewer, including all integers or ranges derivable there between. The nucleic acid molecules are typically synthetic. The term “synthetic” refers to nucleic acid molecule that is isolated and not produced naturally in a cell. In certain aspects all or part of the sequence and/or chemical structure deviates from a naturally-occurring nucleic acid molecule (e.g., an endogenous precursor miRNA or miRNA molecule).

In certain aspects, nucleic acids of the invention do not have an entire sequence that is identical or complementary to a sequence of a naturally-occurring nucleic acids (e.g., miRNA), such molecules may encompass all or part of a naturally-occurring sequence or a complement thereof. It is contemplated, however, that a synthetic nucleic acid administered to a cell may subsequently be modified or altered in the cell such that its structure or sequence is the same or substantially the same as all or part of a non-synthetic or naturally occurring miRNA, such as a mature miRNA sequence. For example, a synthetic nucleic acid may have a sequence that differs from the sequence of a precursor miRNA, but that sequence may be altered once in a cell to be the same as an endogenous, processed miRNA or an inhibitor thereof.

The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many aspects of the invention, a nucleic acid is isolated by virtue of it having been synthesized in vitro separate from endogenous nucleic acids in a cell. It will be understood, however, that isolated nucleic acids may be subsequently mixed or pooled together.

In certain aspects, synthetic miRNA of the invention are RNA or RNA analogs. miRNA inhibitors may be DNA and/or RNA, or analogs thereof. miRNA and miRNA inhibitors of the invention are collectively referred to as “synthetic nucleic acids.”

In some embodiments, a therapeutic nucleic acid can have a miRNA or a synthetic miRNA sequence of between 10-200 to between 17-130 residues, including all values and ranges there between. The present invention concerns miRNA or synthetic miRNA molecules that are, are at least, or are at most 10, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 145, 150, 160, 170, 180, 190, 200 or more residues in length, including any integer or any range there between.

In certain aspects, synthetic nucleic acids have (a) a “miRNA region” whose sequence or binding region from 5′ to 3′ is identical or complementary to all or a segment of a mature miRNA sequence, and (b) a “complementary region” whose sequence from 5′ to 3′ is between 60% and 100% complementary to the miRNA sequence in (a). In certain embodiments, these synthetic nucleic acids are also isolated, as defined above. The term “miRNA region” refers to a region on the synthetic nucleic acid that is at least 75, 80, 85, 90, 95, or 100% identical, including all integers there between, to the entire sequence of a mature, naturally occurring miRNA sequence or a complement thereof In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA, or segment thereof, or complement thereof.

The term “complementary region” or “complement” refers to a region of a nucleic acid or mimetic that is or is at least 60% complementary to the mature, naturally occurring miRNA sequence. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there may be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments of the invention, there are synthetic nucleic acids that are miRNA inhibitors. A miRNA inhibitor is between about 10 to 30 or 17 to 25 nucleotides in length and comprises a 5′ to 3′ sequence that is at least 90% complementary to the 5′ to 3′ sequence of a mature miRNA. In certain embodiments, a miRNA inhibitor molecule is 10, 15, 16, 17, 18, 19, 20, 21, 22, 23, 24, or 25 nucleotides in length, or any range or value there between. Moreover, an miRNA inhibitor may have a sequence (from 5′ to 3′) that is or is at least 70, 75, 80, 85, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. A portion of a nucleic acid sequence can be altered so that it comprises an appropriate percentage of complementarity to the sequence of a mature miRNA or mRNA.

In some embodiments, of the invention, a therapeutic nucleic acid contains one or more design element(s). These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or (iii) noncomplementarity between one or more nucleotides in the last 1 to 5 residues at the 3′ end of the complementary region and the corresponding nucleotides of the miRNA region. A variety of design modifications are known in the art, see below.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an aminohexyl phosphate group, an acetyl group, 2′O-Me (2′oxygen-methyl), DMTO (4,4′-dimethoxytrityl with oxygen), fluorescein, a thiol, or acridine, though other replacement groups are well known to those of skill in the art and can be used as well. This design element can also be used with a miRNA inhibitor.

In certain aspects, a synthetic miRNA can have one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there is one or more sugar modifications in the last 1, 2, 3, 4, 5, 6 or more residues of the complementary region, or any range derivable there between. It will be understood that the terms “first” and “last” are with respect to the order of residues from the 5′ end to the 3′ end of the region. In particular embodiments, the sugar modification is a 2′O-Me modification, a 2′F modification, a 2′H modification, a 2′amino modification, a 4′thioribose modification or a phosphorothioate modification on the carboxy group linked to the carbon at position 6′. In further embodiments, there is one or more sugar modifications in the first or last 2 to 4 residues of the complementary region or the first or last 4 to 6 residues of the complementary region. This design element can also be used with an miRNA inhibitor. Thus, an miRNA inhibitor can have this design element and/or a replacement group on the nucleotide at the 5′ terminus, as discussed above.

In other embodiments of the invention, the therapeutic nucleic acid can have one or more nucleotides in the last 1 to 5 residues at the 3′ end of the complementary region that are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that therapeutic nucleic acids of the invention have one or more of the replacement, sugar modification, or noncomplementarity designs. In certain cases, synthetic nucleic acid molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there can be a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA or inhibitor region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

Methods of the invention include reducing or eliminating activity of one or more miRNAs in a cell comprising introducing into a cell a therapeutic nucleic acid, such as a miRNA inhibitor, (the description of miRNA, where appropriate, also will refer to a miRNA inhibitor); or supplying or enhancing the activity of one or more miRNAs in a cell. The present invention also concerns inducing certain cellular characteristics by providing to a cell a particular nucleic acid, such as a specific therapeutic nucleic acid molecule or a miRNA inhibitor molecule. The therapeutic nucleic acid or miRNA inhibitor need not be synthetic. They may have a sequence that is identical to a naturally occurring miRNA or they may not have any design modifications. In certain embodiments, the therapeutic nucleic acid and/or the miRNA inhibitor are synthetic, as discussed above.

In certain aspects, a particular nucleic acid molecule provided to the cell is understood to correspond to a particular miRNA in the cell, and thus, the miRNA in the cell is referred to as the “corresponding miRNA.” In situations in which a named miRNA molecule is introduced into a cell, the corresponding miRNA will be understood to be the induced or inhibited miRNA function. It is contemplated, however, that the therapeutic nucleic acid introduced into a cell is not a mature miRNA but is capable of becoming or functioning as a mature miRNA under the appropriate physiological conditions. In cases in which a particular corresponding miRNA is being inhibited by a miRNA inhibitor, the particular miRNA will be referred to as the “targeted miRNA.” It is contemplated that multiple corresponding or targeted or combinations of miRNAs may be involved. In particular embodiments, more than one therapeutic nucleic acid is introduced into a cell. Moreover, in other embodiments, more than one miRNA inhibitor is introduced into a cell. Furthermore, a combination of therapeutic nucleic acid(s) and miRNA inhibitor(s) may be introduced into a cell. The inventors contemplate that a combination of therapeutic nucleic acids may act at one or more points in cellular pathways of cells and that such combination may have increased efficacy on the target cell while not adversely effecting normal or non-targeted cells. Thus, a combination of therapeutic nucleic acids may have a minimal adverse effect on a subject or patient while supplying a sufficient therapeutic effect, such as amelioration of a condition, growth inhibition of a cell, death of a targeted cell, alteration of cell phenotype or physiology, slowing of cellular growth, sensitization to a second therapy, sensitization to a particular therapy, and the like.

Methods include identifying a cell or patient in need of inducing those cellular characteristics. Also, it will be understood that an amount of a therapeutic nucleic acid that is provided to a cell or organism is an “effective amount,” which refers to an amount needed (or a sufficient amount) to achieve a desired goal, such as inducing a particular cellular characteristic(s) or reducing cancer growth or killing cancer cells or alleviating symptoms associated with a cancer.

In certain aspects methods can include providing or introducing to a cell a nucleic acid molecule corresponding to a mature miRNA in the cell in an amount effective to achieve a desired physiological result. Moreover, methods can involve providing synthetic or nonsynthetic therapeutic nucleic acids. It is contemplated that in these embodiments, that methods may or may not be limited to providing only one or more synthetic molecules or only one or more nonsynthetic molecules. Thus, in certain embodiments, methods may involve providing both synthetic and nonsynthetic therapeutic nucleic acids. In this situation, a cell or cells are most likely provided a synthetic molecule corresponding to a particular miRNA and a nonsynthetic molecule corresponding to a different miRNA or an inhibitor thereof. Furthermore, any method articulated using a list of miRNA targets using Markush group language may be articulated without the Markush group language and a disjunctive article (i.e., or) instead, and vice versa.

In some embodiments, there is a method for reducing or inhibiting cell proliferation, propagation, or renewal in a cell comprising introducing into or providing to the cell an effective amount of (i) a therapeutic nucleic acid or (ii) a synthetic or nonsynthetic molecule that corresponds to a miRNA sequence. In certain embodiments the methods involve introducing into the cell an effective amount of (i) a miRNA inhibitor molecule having a 5′ to 3′ sequence that is at least 90% complementary to the 5′ to 3′ sequence of one or more miRNA.

Certain aspects of the invention include methods of treating a pathologic condition, such as cancer or precancerous conditions. In one aspect, the method comprises contacting a target cell with one or more nucleic acids comprising at least one nucleic acid segment having all or a portion of a miRNA sequence or a complement thereof. The segment may be 5, 6, 7, 8, 9, 10, 11, 12, 13, 14, 15, 16, 17, 18, 19, 20, 21, 22, 23, 24, 25, 30 or more nucleotides or nucleotide analog, including all integers there between. An aspect of the invention includes the modulation of gene expression, miRNA expression or function or mRNA expression or function within a target cell, such as a prostate cancer cell or CSC.

Typically, an endogenous gene, miRNA or mRNA is modulated in the cell. In certain aspects, a therapeutic nucleic acid sequence comprises at least one segment that is at least 70, 75, 80, 85, 90, 95, or 100% identical in nucleic acid sequence to one or more miRNA or gene sequence or complement thereof Modulation of the expression or processing of a gene, miRNA, or mRNA of the cell or a virus can be through modulation of the processing of a nucleic acid, such processing including transcription, transportation and/or translation with in a cell. Modulation may also be effected by the inhibition or enhancement of miRNA activity with a cell, tissue, or organ. Such processing may affect the expression of an encoded product or the stability of the mRNA.

It will be understood in methods of the invention that a cell or other biological matter such as an organism (including patients) can be provided a therapeutic nucleic acid corresponding to or targeting a particular miRNA by administering to the cell or organism a nucleic acid molecule that functions as the corresponding miRNA once inside the cell. Thus, it is contemplated that a nucleic acid is provided such that it becomes processed into a mature and active miRNA once it has access to the cell's processing machinery. In certain aspects, it is specifically contemplated that the miRNA molecule provided is not a mature molecule but a nucleic acid molecule that can be processed into the mature miRNA or its functional equivalent once it is accessible to processing machinery.

The term “nonsynthetic” in the context of miRNA means that the miRNA is not “synthetic,” as defined herein. Furthermore, it is contemplated that in embodiments of the invention that concern the use of synthetic miRNAs, the use of corresponding nonsynthetic miRNAs is also considered an aspect of the invention, and vice versa. It will be understood that the term “providing” an agent is used to include “administering” the agent to a patient.

In certain embodiments, methods also include targeting an miRNA in a cell or organism. The term “targeting a miRNA” means a nucleic acid of the invention will be employed so as to regulate a selected miRNA. In some embodiments the regulation is achieved with a synthetic or non-synthetic nucleic acid that corresponds to the targeted miRNA, which effectively provides the function of the targeted miRNA to the cell or organism (positive regulation). In other embodiments, the modulation is achieved with a miRNA inhibitor, which effectively inhibits the targeted miRNA in the cell or organism (negative regulation).

In certain methods of the invention, there is a further step of administering a therapeutic nucleic acid to a cell, tissue, organ, or organism (collectively “biological matter”) in need of treatment related to modulation of the targeted miRNA or in need of the physiological or biological results discussed herein (such as with respect to a particular cellular pathway involved in cancer or CSC cycle). Consequently, in some methods of the invention there is a step of identifying a patient in need of treatment that can be provided by the therapeutic nucleic acids of the invention. It is contemplated that an effective amount of a therapeutic nucleic acid can be administered, in some embodiments. In certain aspects, there is a therapeutic benefit conferred on the biological matter, where a “therapeutic benefit” refers to an improvement in the one or more conditions or symptoms associated with a disease or condition or an improvement in the prognosis, duration, or status with respect to the disease. It is contemplated that a therapeutic benefit includes, but is not limited to, a decrease in pain, a decrease in morbidity, a decrease in a symptom. For example, with respect to cancer, it is contemplated that a therapeutic benefit can be inhibition of tumor growth, prevention of metastasis, reduction in number of metastases, inhibition of cancer cell proliferation, induction of cell death in cancer cells, inhibition of angiogenesis near cancer cells, induction of apoptosis of cancer cells, reduction in pain, reduction in risk of recurrence, induction of chemo- or radiosensitivity in cancer cells, prolongation of life, and/or delay of death directly or indirectly related to cancer.

Furthermore, it is contemplated that the nucleic acid compositions may be provided as part of a therapy to a patient, in conjunction with traditional therapies or preventative agents. Moreover, it is contemplated that any method discussed in the context of therapy may be applied as preventatively, particularly in a patient identified to be potentially in need of the therapy or at risk of the condition or disease for which a therapy is needed.

In addition, methods of the invention concern employing one or more nucleic acid corresponding to a miRNA and a therapeutic drug. The nucleic acid can enhance the effect or efficacy of the drug, reduce any side effects or toxicity, modify its bioavailability, and/or decrease the dosage or frequency needed. In certain embodiments, the therapeutic drug is a cancer therapeutic. Consequently, in some embodiments, there is a method of treating a HPV related precancer or cancer in a patient comprising administering to the patient a cancer therapeutic (i.e., a second therapeutic) and an effective amount of at least one nucleic acid molecule that improves the efficacy of the cancer therapeutic or protects non-cancer cells. Cancer therapies also include a variety of combination therapies with both chemical and radiation based treatments.

Generally, inhibitors of miRNAs can be given to decrease the activity of a miRNA and further regulate a nucleic acid targeted by the miRNA. Similarly, nucleic acid molecules corresponding to the mature miRNA can be given to achieve the opposite effect as compared to when inhibitors of the miRNA are given. Methods of the invention are generally contemplated to include providing or introducing one or more different nucleic acid molecules corresponding to one or more different miRNA molecules. It is contemplated that the following, at least the following, or at most the following number of different nucleic acid or miRNA molecules may be detected, assessed, provided or introduced: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, including any value or range derivable there between.

### III. Pharmaceutical Formulations and Delivery

Methods of the present invention include the delivery of an effective amount of a therapeutic nucleic acid or an expression construct comprising or encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease.

A. Administration

In certain embodiments, it is desired to kill cells, inhibit cell growth, inhibit metastasis, decrease tumor or tissue size, and/or reverse or reduce the malignant or disease phenotype of cells. The routes of administration will vary, naturally, with the location and nature of the lesion or site to be targeted, and include, e.g., intradermal, subcutaneous, regional, parenteral, intravenous, intramuscular, intranasal, systemic, and oral administration and formulation. Injection or perfusion of a therapeutic nucleic acid is specifically contemplated for discrete, solid, accessible precancers or cancers, or other accessible target areas. Local, regional, or systemic administration also may be appropriate.

In the case of surgical intervention, the present invention may be used preoperatively, to render an inoperable lesion subject to resection. Alternatively, the present invention may be used at the time of surgery, and/or thereafter, to treat residual or metastatic disease. For example, a resected tumor bed may be injected or perfused with a formulation comprising a therapeutic nucleic acid or combinations thereof. Administration may be continued post-resection, for example, by leaving a catheter implanted at the site of the surgery. Periodic post-surgical treatment also is envisioned. Continuous perfusion of an expression construct or a viral construct also is contemplated.

Continuous administration also may be applied where appropriate, for example, where a tumor or other undesired affected area is excised and the tumor bed or targeted site is treated to eliminate residual, microscopic disease. Delivery via syringe or catherization is contemplated. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well and often depend on the type of lesion, location, immune condition, target site, disease progression, and health and age of the patient. Certain tumor types will require more aggressive treatment. The clinician will be best suited to make such decisions based on the known efficacy and toxicity (if any) of the therapeutic formulations.

In certain embodiments, the lesion or affected area being treated may not, at least initially, be resectable. Treatments with compositions of the invention may increase the resectability of the lesion due to shrinkage at the margins or by elimination of certain particularly invasive portions. Following treatments, resection may be possible. Additional treatments subsequent to resection may serve to eliminate microscopic residual disease at the tumor or targeted site.

Treatments may include various “unit doses.” A unit dose is defined as containing a predetermined quantity of a therapeutic composition(s). The quantity to be administered, and the particular route and formulation, are within the skill of those in the clinical arts. A unit dose need not be administered as a single injection but may comprise continuous infusion over a set period of time. With respect to a viral component of the present invention, a unit dose may conveniently be described in terms of ng, μg, or mg of miRNA or miRNA mimetic. Alternatively, the amount specified may be the amount administered as the average daily, average weekly, or average monthly dose.

A therapeutic nucleic acid can be administered to the patient in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 ng, μg or mg, or more, or any range derivable therein. Alternatively, the amount specified may be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to tumor size or patient surface area).

B. Injectable Compositions and Formulations

In some embodiments, the method for the delivery of a therapeutic nucleic acid or an expression construct encoding such or combinations thereof is via local or systemic administration. However, the pharmaceutical compositions disclosed herein may also be administered parenterally, subcutaneously, intratracheally, intravenously, intradermally, intramuscularly, or even intraperitoneally as described in U.S. Pat. Nos. 5,543,158; 5,641,515 and 5,399,363 (each specifically incorporated herein by reference in its entirety).

Injection of nucleic acids may be delivered by syringe or any other method used for injection of a solution, as long as the nucleic acid and any associated components can pass through the particular gauge of needle required for injection. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, mixtures thereof, and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). Typically, the form must be sterile and must be fluid to the extent that easy syringability exists. It must be stable under the conditions of manufacture and storage and preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain formulations, a water-based formulation is employed while in others, it may be lipid-based. In particular embodiments of the invention, a composition comprising a nucleic acid of the invention is in a water-based formulation. In other embodiments, the formulation is lipid based.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral, intralesional, and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

As used herein, a “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human.

The nucleic acid(s) are administered in a manner compatible with the dosage formulation, and in such amount as will be therapeutically effective. The quantity to be administered depends on the subject to be treated, including, e.g., the aggressiveness of the disease or cancer, the size of any tumor(s) or lesions, the previous or other courses of treatment. Precise amounts of active ingredient required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

C. Combination Treatments

In certain embodiments, the compositions and methods of the present invention involve a therapeutic nucleic acid, or expression construct encoding such. These compositions can be used in combination with a second therapy to enhance the effect of the miRNA therapy, or increase the therapeutic effect of another therapy being employed. These compositions would be provided in a combined amount effective to achieve the desired effect, such as the killing of a cancer cell and/or the inhibition of cellular hyperproliferation. This process may involve contacting the cells with the therapeutic nucleic acid or second therapy at the same or different time. This may be achieved by contacting the cell with one or more compositions or pharmacological formulation that includes or more of the agents, or by contacting the cell with two or more distinct compositions or formulations, wherein one composition provides (1) therapeutic nucleic acid; and/or (2) a second therapy. A second composition or method may be administered that includes a chemotherapy, radiotherapy, surgical therapy, immunotherapy or gene therapy.

It is contemplated that one may provide a patient with the miRNA therapy and the second therapy within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed, for example miRNA therapy is “A” and a second therapy is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the vector or any protein or other agent. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

In specific aspects, it is contemplated that a second therapy, such as chemotherapy, radiotherapy, immunotherapy, surgical therapy or other gene therapy, is employed in combination with the miRNA therapy, as described herein.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

a. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents.

b. Antimetabolites

Antimetabolites disrupt DNA and RNA synthesis. Unlike alkylating agents, they specifically influence the cell cycle during S phase. They have been used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

c. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms, these compounds are administered through bolus injections intravenously at doses ranging from 25-75 mg/m² at 21 day intervals for adriamycin, to 35-100 mg/m² for etoposide intravenously or orally.

d. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

e. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. Radiotherapy may comprise the use of radiolabeled antibodies to deliver doses of radiation directly to the cancer site (radioimmunotherapy). Once injected into the body, the antibodies actively seek out the cancer cells, which are destroyed by the cell-killing (cytotoxic) action of the radiation. This approach can minimize the risk of radiation damage to healthy cells.

Stereotactic radio-surgery (gamma knife) for brain and other tumors does not use a knife, but very precisely targeted beams of gamma radiotherapy from hundreds of different angles. Only one session of radiotherapy, taking about four to five hours, is needed. For this treatment a specially made metal frame is attached to the head. Then, several scans and x-rays are carried out to find the precise area where the treatment is needed. During the radiotherapy for brain tumors, the patient lies with their head in a large helmet, which has hundreds of holes in it to allow the radiotherapy beams through. Related approaches permit positioning for the treatment of tumors in other areas of the body.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor or disease cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. Nos. 5,830,880 and 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). A non-limiting list of several known anti-cancer immunotherapeutic agents and their targets includes (Generic Name/Target) Cetuximab/EGFR, Panitumuma/EGFR, Trastuzumab/erbB2 receptor, Bevacizumab/VEGF, Alemtuzumab/CD52, Gemtuzumab ozogamicin/CD33, Rituximab/CD20, Tositumomab/CD20, Matuzumab/EGFR, Ibritumomab tiuxetan/CD20, Tositumomab/CD20, HuPAM4/MUC1, MORAb-009/Mesothelin, G250/carbonic anhydrase IX, mAb 8H9/8H9 antigen, M195/CD33, Ipilimumab/CTLA4, HuLuc63/CS1, Alemtuzumab/CD53, Epratuzumab/CD22, BC8/CD45, HuJ591/Prostate specific membrane antigen, hA20/CD20, Lexatumumab/TRAIL receptor-2, Pertuzumab/HER-2 receptor, Mik-beta-1/IL-2R, RAV12/RAAG12, SGN-30/CD30, AME-133v/CD20, HeFi-1/CD30, BMS-663513/CD137, Volociximab/anti-α5β1 integrin, GC1008/TGFβ, HCD122/CD40, Siplizumab/CD2, MORAb-003/Folate receptor alpha, CNTO 328/IL-6, MDX-060/CD30, Ofatumumab/CD20, and SGN-33/CD33. It is contemplated that one or more of these therapies may be employed with the miRNA therapies described herein.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

4. Gene Therapy

In yet another embodiment, a combination treatment involves gene therapy in which a therapeutic polynucleotide is administered before, after, or at the same time as one or more therapeutic miRNA. Delivery of a therapeutic polypeptide or encoding nucleic acid in conjunction with a miRNA may have a combined therapeutic effect on target tissues. A variety of proteins are encompassed within the invention, some of which are described below. Various genes that may be targeted for gene therapy of some form in combination with the present invention include, but are not limited to inducers of cellular proliferation, inhibitors of cellular proliferation, regulators of programmed cell death, cytokines and other therapeutic nucleic acids or nucleic acid that encode therapeutic proteins.

The tumor suppressor oncogenes function to inhibit excessive cellular proliferation. The inactivation of these genes destroys their inhibitory activity, resulting in unregulated proliferation. The tumor suppressors (e.g., therapeutic polypeptides) p53, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p16INK4 has been biochemically characterized as a protein that specifically binds to and inhibits CDK4, and thus may regulate Rb phosphorylation (Serrano et al., 1993; Serrano et al., 1995). Since the p16INK4 protein is a CDK4 inhibitor (Serrano, 1993), deletion of this gene may increase the activity of CDK4, resulting in hyperphosphorylation of the Rb protein. p16 also is known to regulate the function of CDK6.

p16INK4 belongs to a newly described class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p16INK4 gene are frequent in human tumor cell lines. This evidence suggests that the p16INK4 gene is a tumor suppressor gene. This interpretation has been challenged, however, by the observation that the frequency of the p16INK4 gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). Restoration of wild-type p16INK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells. (Marsters et al., 1999).

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

This application incorporates U.S. application Ser. No. 11/349,727 filed on Feb. 8, 2006 claiming priority to U.S. Provisional Application Ser. No. 60/650,807 filed Feb. 8, 2005 herein by references in its entirety.

### IV. Therapeutic Nucleic Acids

Therapeutic nucleic acids typically include segments of sequence or complementary sequences to microRNA (“miRNA” or “miR”) molecules, which are generally 21 to 22 nucleotides in length, though lengths of 17 and up to 24 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) become part of a large complex to down-regulate a particular target gene. Examples of animal miRNAs include those that imperfectly basepair with the target, which halts translation (Olsen et al., 1999; Seggerson et al., 2002). siRNA molecules also are processed by Dicer, but from a long, double-stranded RNA molecule. siRNAs are not naturally found in animal cells, but they can direct the sequence-specific cleavage of an mRNA target through a RNA-induced silencing complex (RISC) (Denli et al., 2003).

In certain aspects, therapeutic nucleic acids of the invention are RNA or RNA analogs. miRNA inhibitors may be DNA or RNA, or analogs thereof. In other aspects, an miRNA inhibitor can be a protein or a polypeptide that interacts with an endogenous miRNA or processing.

In some embodiments, there is a synthetic or isolated miRNA having a length of between 17 and 130 residues. The present invention concerns synthetic miRNA molecules that are, are at least, or are at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 145, 150, 160, 170, 180, 190, 200 or more residues in length, including any integer or any range derivable therein.

In certain embodiments, therapeutic nucleic acids have (a) a “miRNA region” whose sequence from 5′ to 3′ is identical to all or a segment of a mature miRNA sequence, and (b) a “complementary region” whose sequence from 5′ to 3′ is between 60% and 100% complementary to the miRNA sequence. In certain embodiments, these synthetic miRNA are also isolated, as defined above. The term “miRNA region” refers to a region on the synthetic miRNA that is at least 75, 80, 85, 90, 95, or 100% identical, including all integers there between, to the entire sequence of a mature, naturally occurring miRNA sequence. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA. Alternatively, the miRNA region can comprise 18, 19, 20, 21, 22, 23, 24 or more nucleotide positions in common with a naturally-occurring miRNA as compared by sequence alignment algorithms and methods well known in the art.

The term “complementary region” refers to a region of a synthetic miRNA that is or is at least 60% complementary to the mature, naturally occurring miRNA sequence that the miRNA region is identical to. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there may be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments of the invention, there are therapeutic nucleic acids that are miRNA inhibitors. A miRNA inhibitor is between about 17 to 25 nucleotides in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, or 25 nucleotides in length, or any range derivable therein. Moreover, a miRNA inhibitor has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. One of skill in the art could use a portion of the probe sequence that is complementary to the sequence of a mature miRNA as the sequence for a miRNA inhibitor. Moreover, that portion of the probe sequence can be altered so that it is still 90% complementary to the sequence of a mature miRNA.

In some embodiments, of the invention, a therapeutic nucleic acid contains one or more design elements. These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or, (iii) noncomplementarity between one or more nucleotides in the last 1 to 5 residues at the 3′ end of the complementary region and the corresponding nucleotides of the miRNA region.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2′O-Me (2′oxygen-methyl), DMTO (4,4′-dimethoxytrityl with oxygen), fluorescein, a thiol, or acridine, though other replacement groups are well known to those of skill in the art and can be used as well. This design element can also be used with a miRNA inhibitor.

Additional embodiments concern a synthetic miRNA having one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there is one or more sugar modifications in the last 1, 2, 3, 4, 5, 6 or more residues of the complementary region, or any range derivable therein, have a sugar modification. It will be understood that the terms “first” and “last” are with respect to the order of residues from the 5′ end to the 3′ end of the region. In particular embodiments, the sugar modification is a 2′O-Me modification, a 2′F modification, a 2′H modification, a 2′amino modification, a 4′thioribose modification or a phosphorothioate modification on the carboxy group linked to the carbon at position 6′, or combinations thereof. In further embodiments, there is one or more sugar modifications in the first or last 2 to 4 residues of the complementary region or the first or last 4 to 6 residues of the complementary region. This design element can also be used with a miRNA inhibitor. Thus, a miRNA inhibitor can have this design element and/or a replacement group on the nucleotide at the 5′ terminus, as discussed above.

In other embodiments of the invention, there is a synthetic miRNA in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention have one or more of the replacement, sugar modification, or noncomplementarity designs. In certain cases, synthetic RNA molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there is a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

In some embodiments of the invention, methods and compositions involving miRNA may concern miRNA and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA containing vectors, control nucleic acids, and other probes and primers. In many embodiments, miRNA are 19-24 nucleotides in length, while miRNA probes are 5, 10, 15, 19, 20, 25, 30, to 35 nucleotides in length, including all values and ranges there between, depending on the length of the processed miRNA and any flanking regions added. miRNA precursors are generally between 62 and 110 nucleotides in humans.

Nucleic acids of the invention may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or 110 contiguous nucleotides. It is further understood that the length of complementarity within a precursor miRNA or between a miRNA probe and a miRNA or a miRNA gene are such lengths. Moreover, the complementarity may be expressed as a percentage, meaning that the complementarity between a probe and its target is 90% identical or greater over the length of the probe. In some embodiments, complementarity is or is at least 90%, 95% or 100% identical. In particular, such lengths may be applied to any nucleic acid comprising a nucleic acid sequence identified in any of SEQ ID NOs disclosed herein.

The term “recombinant” may be used and this generally refers to a molecule that has been manipulated in vitro or that is a replicated or expressed product of such a molecule.

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary miRNA probes or nucleic acids of the invention can include, can be or can be at least 60, 65, 70, 75, 80, 85, 90, 95, 96, 97, 98, 99 or 100% complementary to their target.

Nucleic acids of the invention may be made by any technique known to one of ordinary skill in the art, such as for example, chemical synthesis, enzymatic production or biological production. It is specifically contemplated that miRNA probes of the invention are chemically synthesized.

In some embodiments of the invention, miRNAs are recovered or isolated from a biological sample. The miRNA may be recombinant or it may be natural or endogenous to the cell (produced from the cell's genome). It is contemplated that a biological sample may be treated in a way so as to enhance the recovery of small RNA molecules such as miRNA. U.S. patent application Ser. No. 10/667,126 describes such methods and it is specifically incorporated by reference herein. Generally, methods involve lysing cells with a solution having guanidinium and a detergent.

A. Isolation of Nucleic Acids

Nucleic acids may be isolated using techniques well known to those of skill in the art, though in particular embodiments, methods for isolating small nucleic acid molecules, and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography.

If miRNA from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased, such as from C.B.S. Scientific Co., Inc. or Scie-Plas.

Methods may involve the use of organic solvents and/or alcohol to isolate nucleic acids, particularly miRNA used in methods and compositions of the invention. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column has worked particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for form a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, f) capturing the miRNA molecules. Typically the sample is dried down and resuspended in a liquid and volume appropriate for subsequent manipulation.

B. Preparation of Nucleic Acids

Alternatively, nucleic acid synthesis is performed according to standard methods. See, for example, Itakura and Riggs (1980). Additionally, U.S. Pat. Nos. 4,704,362, 5,221,619, and 5,583,013 each describe various methods of preparing synthetic nucleic acids. Non-limiting examples of a synthetic nucleic acid (e.g., a synthetic oligonucleotide), include a nucleic acid made by in vitro chemically synthesis using phosphotriester, phosphite, or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al., 2001, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Recombinant methods for producing nucleic acids in a cell are well known to those of skill in the art. These include the use of vectors (viral and non-viral), plasmids, cosmids, and other vehicles for delivering a nucleic acid to a cell, which may be the target cell (e.g., a cancer cell) or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

In certain embodiments, the present invention concerns nucleic acid molecules that are not synthetic. In some embodiments, the nucleic acid molecule has a chemical structure of a naturally occurring nucleic acid and a sequence of a naturally occurring nucleic acid, such as the exact and entire sequence of a single stranded primary miRNA (see Lee, 2002), a single-stranded precursor miRNA, or a single-stranded mature miRNA. In addition to the use of recombinant technology, such non-synthetic nucleic acids may be generated chemically, such as by employing technology used for creating oligonucleotides.

C. Labels and Labeling Techniques

In some embodiments, the present invention concerns miRNA that are directly or indirectly labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In many embodiments of the invention, the label is non-radioactive. Generally, nucleic acids may be labeled by adding labeled nucleotides (one-step process) or adding nucleotides and labeling the added nucleotides (two-step process).

In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference.

In other embodiments, an unlabeled nucleotide or nucleotides is catalytically added to a miRNA, and the unlabeled nucleotide is modified with a chemical moiety that enables it to be subsequently labeled. In embodiments of the invention, the chemical moiety is a reactive amine such that the nucleotide is an amine-modified nucleotide. Examples of amine-modified nucleotides are well known to those of skill in the art, many being commercially available such as from Ambion, Sigma, Jena Bioscience, and TriLink.

In contrast to labeling of cDNA during its synthesis, the issue for labeling miRNA is how to label the already existing molecule. The present invention concerns the use of an enzyme capable of using a di- or tri-phosphate ribonucleotide or deoxyribonucleotide as a substrate for its addition to a miRNA. Moreover, in specific embodiments, it involves using a modified di- or tri-phosphate ribonucleotide, which is added to the 3′ end of a miRNA. The source of the enzyme is not limiting. Examples of sources for the enzymes include yeast, gram-negative bacteria such as E. coli, Lactococcus lactis, and sheep pox virus.

Enzymes capable of adding such nucleotides include, but are not limited to, poly(A) polymerase, terminal transferase, and polynucleotide phosphorylase. In specific embodiments of the invention, a ligase is contemplated as not being the enzyme used to add the label, and instead, a non-ligase enzyme is employed.

Terminal transferase catalyzes the addition of nucleotides to the 3′ terminus of a nucleic acid.

Polynucleotide phosphorylase can polymerize nucleotide diphosphates without the need for a primer.

Labels on miRNA or miRNA probes may be colorimetric (includes visible and UV spectrum, including fluorescent), luminescent, enzymatic, or positron emitting (including radioactive). The label may be detected directly or indirectly. Radioactive labels include ¹²⁵I, ³²P, ³³P, and ³⁵S. Examples of enzymatic labels include alkaline phosphatase, luciferase, horseradish peroxidase, and β-galactosidase. Labels can also be proteins with luminescent properties, e.g., green fluorescent protein and phycoerythrin.

The colorimetric and fluorescent labels contemplated for use as conjugates include, but are not limited to, Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides are available from Molecular Probes, and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences, such as Cy3-UTP and Cy5-UTP.

Examples of fluorescently labeled deoxyribonucleotides include Dinitrophenyl (DNP)-11-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP.

It is contemplated that nucleic acids may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference).

Alternatively, the label may not be detectable per se, but indirectly detectable or allowing for the isolation or separation of the targeted nucleic acid. For example, the label could be biotin, digoxigenin, polyvalent cations, chelator groups and the other ligands, include ligands for an antibody.

A number of techniques for visualizing or detecting labeled nucleic acids are readily available. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC (Griffey et al., 1997), spectroscopy, capillary gel electrophoresis (Cummins et al., 1996), spectroscopy; mass spectroscopy; radiological techniques; and mass balance techniques.

When two or more differentially colored labels are employed, fluorescent resonance energy transfer (FRET) techniques may be employed to characterize association of one or more nucleic acid. Furthermore, a person of ordinary skill in the art is well aware of ways of visualizing, identifying, and characterizing labeled nucleic acids, and accordingly, such protocols may be used as part of the invention. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

### V. Evaluation of miRNA Levels

It is contemplated that a number of assays could be employed to analyze miRNAs, their activities, and their effects. Such assays include, but are not limited to, array hybridization, solution hybridization, nucleic amplification, polymerase chain reaction, quantitative PCR, RT-PCR, in situ hybridization, Northern hybridization, hybridization protection assay (HPA) (GenProbe), branched DNA (bDNA) assay (Chiron), rolling circle amplification (RCA), single molecule hybridization detection (US Genomics), Invader assay (ThirdWave Technologies), and/or Oligo Ligation Assay (OLA), hybridization, and array analysis.

U.S. patent application Ser. No. 11/141,707, filed May 31, 2005; Ser. No. 11/857,948, filed Sep. 19, 2007; Ser. No. 11/273,640, filed Nov. 14, 2005 and provisional patent application 60/869,295, filed Dec. 8, 2006 are incorporated by reference in their entirety.

A. Sample Preparation

While endogenous miRNA is contemplated for use with compositions and methods of the invention, recombinant miRNA—including nucleic acids that are complementary or identical to endogenous miRNA or precursor miRNA—can also be handled and analyzed as described herein. Samples may be biological samples, in which case, they can be from lavage, biopsy, fine needle aspirates, exfoliates, blood, sputum, tissue, organs, semen, saliva, tears, urine, cerebrospinal fluid, body fluids, hair follicles, skin, or any sample containing or constituting biological cells. In certain embodiments, samples may be, but are not limited to, fresh, frozen, fixed, formalin fixed, preserved, RNAlater preserved, paraffin embedded, or formalin fixed and paraffin embedded. Alternatively, the sample may not be a biological sample, but be a chemical mixture, such as a cell-free reaction mixture (which may contain one or more biological enzymes).

B. Differential Expression Analyses

Methods of the invention can be used to detect differences in miRNA expression or levels between two samples, or a sample and a reference (e.g., a tissue reference or a digital reference representative of a non-cancerous state). Specifically contemplated applications include identifying and/or quantifying differences between miRNA from a sample that is normal and from a sample that is not normal, between a cancerous condition and a non-cancerous condition, or between two differently treated samples (e.g., a pretreatment versus a posttreatment sample). Also, miRNA may be compared between a sample believed to be susceptible to a particular therapy, disease, or condition and one believed to be not susceptible or resistant to that therapy, disease, or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of a disease or condition of which a component is or may or may not be genetic or caused by a hyperproliferative or neoplastic cell or cells.

It is specifically contemplated that the invention can be used to evaluate differences between cell types (normal cells from stem cells); stages of disease, such as between hyperplasia, neoplasia, pre-cancer and cancer, or between a primary tumor and a metastasized tumor.

Phenotypic traits also include characteristics such as longevity, morbidity, susceptibility or receptivity to particular drugs or therapeutic treatments (drug efficacy), and risk of drug toxicity.

In certain embodiments, miRNA profiles may be generated to evaluate and correlate those profiles with pharmacokinetics. For example, miRNA profiles may be created and evaluated for patient tumor and blood samples prior to the patient's being treated or during treatment to determine if there are miRNAs whose expression correlates with the outcome of treatment. Identification of differential miRNAs can lead to a diagnostic assay involving them that can be used to evaluate tumor and/or blood samples to determine what drug regimen the patient should be provided. In addition, it can be used to identify or select patients suitable for a particular clinical trial. If a miRNA profile is determined to be correlated with drug efficacy or drug toxicity that may be relevant to whether that patient is an appropriate patient for receiving the drug or for a particular dosage of the drug.

In addition to the above assay, blood samples from patients can be evaluated to identify a disease or a condition based on miRNA levels, such as metastatic disease. A diagnostic assay can be created based on the profiles that doctors can use to identify individuals with a disease or who are at risk to develop a disease. Alternatively, treatments can be designed based on miRNA profiling. Examples of such methods and compositions are described in the U.S. Provisional Patent Application entitled “Methods and Compositions Involving miRNA and miRNA Inhibitor Molecules” filed on May 23, 2005, which is hereby incorporated by reference in its entirety.

C. Amplification

Many methods exist for evaluating miRNA levels by amplifying all or part of miRNA nucleic acid sequences such as mature miRNAs, precursor miRNAs, and primary miRNAs. Suitable nucleic acid polymerization and amplification techniques include reverse transcription (RT), polymerase chain reaction (PCR), real-time PCR (quantitative PCR (q-PCR)), nucleic acid sequence-base amplification (NASBA), ligase chain reaction, multiplex ligatable probe amplification, invader technology (Third Wave), rolling circle amplification, in vitro transcription (IVT), strand displacement amplification, transcription-mediated amplification (TMA), RNA (Eberwine) amplification, and other methods that are known to persons skilled in the art. In certain embodiments, more than one amplification method may be used, such as reverse transcription followed by real time PCR (Chen et al., 2005 and/or U.S. patent application Ser. No. 11/567,082, filed Dec. 5, 2006, which are incorporated herein by reference in its entirety).

A typical PCR reaction includes multiple amplification steps, or cycles that selectively amplify target nucleic acid species. Typical PCR reactions include 20 or more cycles of denaturation, annealing, and elongation. In many cases, the annealing and elongation steps can be performed concurrently, in which case the cycle contains only two steps. Since mature miRNAs are single stranded, a reverse transcription reaction (which produces a complementary cDNA sequence) is typically performed prior to PCR reactions. Reverse transcription reactions include the use of, e.g., a RNA-based DNA polymerase (reverse transcriptase) and a primer.

In PCR and q-PCR methods, for example, a set of primers is used for each target sequence. In certain embodiments, the lengths of the primers depends on many factors, including, but not limited to, the desired hybridization temperature between the primers, the target nucleic acid sequence, and the complexity of the different target nucleic acid sequences to be amplified. In certain embodiments, a primer is about 15 to about 35 nucleotides in length. In other embodiments, a primer is equal to or fewer than 15, 20, 25, 30, or 35 nucleotides in length. In additional embodiments, a primer is at least 35 nucleotides in length.

In a further aspect, a forward primer can comprise at least one sequence that anneals to a target miRNA and alternatively can comprise an additional 5′ noncomplementary region. In another aspect, a reverse primer can be designed to anneal to the complement of a reverse transcribed miRNA. The reverse primer may be independent of the miRNA sequence, and multiple miRNAs may be amplified using the same reverse primer. Alternatively, a reverse primer may be specific for a miRNA.

In some embodiments, two or more miRNAs or nucleic acids are amplified in a single reaction volume or multiple reaction volumes. In certain aspects, one or more miRNA or nucleic may be used as a normalization control or a reference nucleic acid for normalization. Normalization may be performed in separate or the same reaction volumes as other amplification reactions. One aspect includes multiplex q-PCR, such as qRT-PCR, which enables simultaneous amplification and quantification of at least one miRNA of interest and at least one reference nucleic acid in one reaction volume by using more than one pair of primers and/or more than one probe. The primer pairs comprise at least one amplification primer that uniquely binds each nucleic acid, and the probes are labeled such that they are distinguishable from one another, thus allowing simultaneous quantification of multiple miRNAs. Multiplex qRT-PCR has research and diagnostic uses, including but not limited to detection of miRNAs for diagnostic, prognostic, and therapeutic applications.

A single combined reaction for q-PCR, may be used to: (1) decreased risk of experimenter error, (2) reduction in assay-to-assay variability, (3) decreased risk of target or product contamination, and (4) increased assay speed. The qRT-PCR reaction may further be combined with the reverse transcription reaction by including both a reverse transcriptase and a DNA-based thermostable DNA polymerase. When two polymerases are used, a “hot start” approach may be used to maximize assay performance (U.S. Pat. Nos. 5,411,876 and 5,985,619). For example, the components for a reverse transcriptase reaction and a PCR reaction may be sequestered using one or more thermoactivation methods or chemical alteration to improve polymerization efficiency (U.S. Pat. Nos. 5,550,044, 5,413,924, and 6,403,341).

To assess the expression of microRNAs, real-time RT-PCR detection can be used to screen nucleic acids or RNA isolated from samples of interest and a related reference such as normal adjacent tissue (NAT) samples.

A panel of amplification targets is chosen for real-time RT-PCR quantification. The selection of the panel or targets can be based on the results of microarray expression analyses, such as mirVana™ miRNA Bioarray V1, Ambion. In one aspect, the panel of targets includes one or more miRNA described herein. One example of a normalization target is 5S rRNA and other can be included. Reverse transcription (RT) reaction components are typically assembled on ice prior to the addition of RNA template. Total RNA template is added and mixed. RT reactions are incubated in an appropriate PCR System at an appropriate temperature (15-30° C., including all values and ranges there between) for an appropriate time, 15 to 30 minutes or longer, then at a temperature of 35 to 42 to 50° C. for 10 to 30 to 60 minutes, and then at 80 to 85 to 95° C. for 5 minutes, then placed on wet ice. Reverse Transcription reaction components typically include nuclease-free water, reverse transcription buffer, dNTP mix, RT Primer, RNase Inhibitor, Reverse Transcriptase, and RNA.

PCR reaction components are typically assembled on ice prior to the addition of the cDNA from the RT reactions. Following assembly of the PCR reaction components a portion of the RT reaction is transferred to the PCR mix. PCR reaction are then typically incubated in an PCR system at an elevated temperature (e.g., 95° C.) for 1 minute or so, then for a number of cycles of denaturing, annealing, and extension (e.g., 40 cycles of 95° C. for 5 seconds and 60° C. for 30 seconds). Results can be analyzed, for example, with SDS V2.3 (Applied Biosystems). Real-time PCR components typically include Nuclease-free water, MgCl₂, PCR Buffer, dNTP mix, one or more primers, DNA Polymerase, cDNA from RT reaction and one or more detectable label.

Software tools such as NormFinder (Andersen et al., 2004) are used to determine targets for normalization with the targets of interest and tissue sample set. For normalization of the real-time RT-PCR results, the cycle threshold (Cₜ) value (a log value) for the microRNA of interest is subtracted from the geometric mean Cₜ value of normalization targets. Fold change can be determined by subtracting the dCₜ normal reference (N) from the corresponding dCₜ sample being evaluated (T), producing a ddCₜ(T-N) value for each sample. The average ddCₜ(T-N) value across all samples is converted to fold change by 2^{ddCt}. The representative p-values are determined by a two-tailed paired Student's t-test from the dCₜ values of sample and normal reference.

D. Nucleic Acid Arrays

Certain aspects of the present invention concerns the preparation and use of miRNA arrays or miRNA probe arrays, which are ordered macroarrays or microarrays of nucleic acid molecules (probes) that are fully or nearly complementary or identical to a plurality of miRNA molecules or precursor miRNA molecules and are positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610;287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO 0138580; WO 0168255; WO 03020898; WO 03040410; WO 03053586; WO 03087297; WO 03091426; WO 03100012; WO 04020085; WO 04027093; EP 373 203; EP 785 280; EP 799 897 and UK 8 803 000; the disclosures of which are all herein incorporated by reference. Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

E. Hybridization

After an array or a set of miRNA probes is prepared and the miRNA in the sample is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in many embodiments is the use of stringent conditions during hybridization. Stringent conditions are known to those of skill in the art.

### VI. Kits

Any of the compositions or components described herein may be comprised in a kit. In a non-limiting example, reagents for isolating miRNA, labeling miRNA, and/or evaluating a miRNA population using an array, nucleic acid amplification, and/or hybridization can be included in a kit, as well reagents for preparation of samples from colon samples. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. In certain aspects, the kit can include amplification reagents. In other aspects, the kit may include various supports, such as glass, nylon, polymeric beads, magnetic beads, and the like, and/or reagents for coupling any probes and/or target nucleic acids. It may also include one or more buffers, such as reaction buffer, labeling buffer, washing buffer, or a hybridization buffer, compounds for preparing the miRNA probes, and components for isolating miRNA. Other kits of the invention may include components for making a nucleic acid array comprising miRNA, and thus, may include, for example, a solid support.

Kits for implementing methods of the invention described herein are specifically contemplated. In some embodiments, there are kits for preparing miRNA for multi-labeling and kits for preparing miRNA probes and/or miRNA arrays. In these embodiments, kit comprise, in suitable container means, 1, 2, 3, 4, 5, 6, 7, 8, 9, 10, 11, 12 or more of the following: 1) poly(A) polymerase; 2) unmodified nucleotides (G, A, T, C, and/or U); 3) a modified nucleotide (labeled or unlabeled); 4) poly(A) polymerase buffer; and, 5) at least one microfilter; 6) label that can be attached to a nucleotide; 7) at least one miRNA probe; 8) reaction buffer; 9) a miRNA array or components for making such an array; 10) acetic acid; 11) alcohol; 12) solutions for preparing, isolating, enriching, and purifying miRNAs or miRNA probes or arrays. Other reagents include those generally used for manipulating RNA, such as formamide, loading dye, ribonuclease inhibitors, and DNase.

In specific embodiments, kits of the invention include an array containing miRNA probes, as described in the application. An array may have probes corresponding to all known miRNAs of an organism or a particular tissue or organ in particular conditions, or to a subset of such probes. The subset of probes on arrays of the invention may be or include those identified as relevant to a particular diagnostic, therapeutic, or prognostic application. For example, the array may contain one or more probes that is indicative or suggestive of (1) a disease or condition (colon cancer), (2) susceptibility or resistance to a particular drug or treatment; (3) susceptibility to toxicity from a drug or substance; (4) the stage of development or severity of a disease or condition (prognosis); and (5) genetic predisposition to a disease or condition.

For any kit embodiment, including an array, there can be nucleic acid molecules that contain or can be used to amplify a sequence that is a variant of, identical to or complementary to all or part of any of SEQ ID NOs described herein. Any nucleic acid discussed above may be implemented as part of a kit.

The components of the kits may be packaged either in aqueous media or in lyophilized form. The container means of the kits will generally include at least one vial, test tube, flask, bottle, syringe or other container means, into which a component may be placed, and preferably, suitably aliquotted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be resuspended in any suitable solvent, such as DMSO.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Such kits may also include components that facilitate isolation of the labeled miRNA. It may also include components that preserve or maintain the miRNA or that protect against its degradation. Such components may be RNase-free or protect against RNases. Such kits generally will comprise, in suitable means, distinct containers for each individual reagent or solution.

A kit will also include instructions for employing the kit components as well the use of any other reagent not included in the kit. Instructions may include variations that can be implemented.

Kits of the invention may also include one or more of the following: Control RNA; nuclease-free water; RNase-free containers, such as 1.5 ml tubes; RNase-free elution tubes; PEG or dextran; ethanol; acetic acid; sodium acetate; ammonium acetate; guanidinium; detergent; nucleic acid size marker; RNase-free tube tips; and RNase or DNase inhibitors.

It is contemplated that such reagents are embodiments of kits of the invention. Such kits, however, are not limited to the particular items identified above and may include any reagent used for the manipulation or characterization of miRNA.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### miRNAs Differentially Expressed in Prostate Cancer Stem Cells

The inventors identified miRNAs that are differentially regulated in prostate CSCs. CSCs were isolated from three, well-characterized human prostate cancer xenograft tumors (LAPC4, LAPC9, and Du145), following tumor growth in mice. LAPC4 and LAPC9 prostate xenograft cells were kindly provided by C. Sawyers (Reiters and Sawyers, 1999). Du145 cells were obtained from ATCC (Manassas, Va., USA) and maintained in culture using RPMI (Invitrogen Corp., Carlsbad, Calif., USA) supplemented with 10% fetal bovine serum (FBS) as recommended by the company. Cells (3×10⁶ per cell type) were injected subcutaneously into the flanks of NOD/SCID mice (non-obese diabetic/severe combined immunodeficiency) (JAX® Mice and Services, Bar Harbor, Me., USA), and tumor growth was measured biweekly with calipers. Tumors were harvested when they reached ˜400 mm³ in size and dissociated into single cells by methods previously described (Patrawala et al., 2006). Briefly, the tumors were minced into 1 mm³ pieces in RPMI+10% FBS, washed with phosphate buffered saline (PBS), and incubated with 1× Accumax (1,200-2,000 U/ml proteolytic activity containing collagenase and DNase; Innovative Cell Technologies Inc., San Diego, Calif., USA) at 10 ml/g tissue. A single cell suspension was obtained by filtering the supernatant through a 40-μM cell strainer (BD Falcon, Bedford, Mass., USA), and viable cells were counted using the erythrosine B dye (ATCC). The cell suspension was gently loaded onto a layer of Histopaque-1077 gradient (Sigma-Aldrich, St. Louis, Mo., USA) and centrifuged at 400 g for 30 minutes at room temperature. Contaminating mouse cells were removed using the MACS Lineage Cell Depletion kit (Cat. No. 130-090-858; Miltenyi Biotec, Inc.; Auburn, Calif., USA).

The most common method for identifying CSCs in a tumor is by the presence of specific cell surface markers that tag normal stem cells in the tissue of origin. Prostate cancer stem cells are CD133+CD44+, the same phenotype as normal prostate stem cells (Collins et al., 2005; Richardson et al., 2004). CD44 identifies CSCs in LAPC9, LAPC4 (lymph node metastasis, Reiters and Sawyers, 2001) and Du145 (brain metastasis, Stone et al., 1978) tumors. CD44 and ITGA2 were shown to identify a subset of highly tumorigenic metastatic cells in prostate cancer xenografts (Patrawala et al., 2006, 2007). Single cell suspensions were stained with fluorescein isothiocyanate- or phycoerythrin-conjugated antibodies against CD44 (LAPC4, LAPC9, Du145) or CD133 (LAPC4) for 15 minutes at 4° C. All antibodies were purchased from BD Biosciences (San Jose, Calif., USA) or Chemicon (Temecula, Calif., USA). CD133⁺ and CD44⁺ cells were sorted using a Beckman-Coulter EPICS Elite fluorescence activated cell-sorter (FACS). The purity of the sorted cell populations was ˜99% as determined by visual examination under a fluorescence microscope and by FACS analysis of the sorted fraction. Cells were harvested by centrifugation and immediately frozen for RNA isolation.

Total RNA was extracted using the mirVana™ miRNA Isolation kit (cat. no. AM1561; Ambion, Inc.; Austin, Tex., USA) according to the manufacturer's recommended protocol. Expression of 137 miRNAs was determined in duplicate by quantitative real-time PCR using (1) RNA from cells derived from LAPC4, LAPC9, and Du145 xenografts and carrying the CD44 or CD133 epitopes and (2) RNA from the population of tumor cells recovered after CSC removal from the respective tumors. Quantitative real-time PCR assays employed TaqMan® MicroRNA Assay reagents (Applied Biosystems, Foster City, Calif., USA). Raw Ct values from CSCs were normalized to those of miR-103, a housekeeping miRNA (Peltier & Latham, 2008), and expressed relative to the normalized values generated in the CSC-depleted prostate tumor cell population (Table 1 and Table 2).

TABLE 1
miRNAs differentially expressed in prostate cancer stem cells
(CSCs) purified from human prostate xenograft tumors of LAPC4,
LAPC9, or DU145 cells, using antibodies against the CD44 surface
antigen, representing prostate cancer stem cells.

| | Relative Expression (log₂) | | |
|---|---|---|---|
| miRNA | LAPC4 | LAPC9 | Du145 |

miRNAs downregulated in CD44(+) cells

| | | | |
|---|---|---|---|
| hsa-let-7a | 0.76 | 1.15 | 1.79 |
| hsa-let-7b | 1.75 | 1.4 | 1.46 |
| hsa-let-7c | 1.05 | NS | NS |
| hsa-let-7e | 0.54 | 0.83 | 0.7 |
| hsa-let-7f | 0.66 | 0.75 | 1.41 |
| hsa-miR-101 | NS | NS | 0.75 |
| hsa-miR-106a | NS | NS | 0.57 |
| hsa-miR-10a | 16.9 | 0.96 | 1.12 |
| hsa-miR-125a | NS | 1.04 | NS |
| hsa-miR-126 | NS | 2.29 | 1.62 |
| hsa-miR-132 | 0.72 | 0.92 | 0.61 |
| hsa-miR-141 | NS | 1.03 | 2.8 |
| hsa-miR-148a | NS | 1.69 | NS |
| hsa-miR-148b | 1.45 | 1.62 | 0.9 |
| hsa-miR-149 | 0.96 | 0.76 | NS |
| hsa-miR-151 | 0.67 | 0.73 | 1.03 |
| hsa-miR-15b | NS | NS | 2.03 |
| hsa-miR-181b | 1.37 | 1.19 | 1.07 |
| hsa-miR-182 | NS | NS | 0.64 |
| hsa-miR-183 | 0.54 | 1.08 | 0.73 |
| hsa-miR-18a | 12.55 | NS | NS |
| hsa-miR-191 | NS | 1.12 | NS |
| hsa-miR-193b | 1.5 | NS | NS |
| hsa-miR-196a | 2.69 | 0.99 | 2.57 |
| hsa-miR-199a | 12.39 | 4.25 | 3.67 |
| hsa-miR-200c | 1.05 | 1.02 | NS |
| hsa-miR-203 | 0.56 | 0.74 | 7.27 |
| hsa-miR-214 | 1.97 | 3.25 | NS |
| hsa-miR-218 | 3.6 | 3.49 | 6.94 |
| hsa-miR-222 | 2.19 | 0.98 | NS |
| hsa-miR-24 | 0.73 | 0.71 | NS |
| hsa-miR-25 | 1.85 | NS | 1.05 |
| hsa-miR-26a | NS | 0.52 | NS |
| hsa-miR-27a | 0.68 | NS | NS |
| hsa-miR-29b | 1.33 | 1.07 | NS |
| hsa-miR-30a-3p | NS | 0.54 | 1.18 |
| hsa-miR-30a-5p | 0.98 | NS | 0.74 |
| hsa-miR-30d | 0.7 | NS | NS |
| hsa-miR-324-5p | 3.45 | NS | NS |
| hsa-miR-331 | NS | NS | 1.35 |
| hsa-miR-335 | 0.77 | 0.52 | NS |
| hsa-miR-340 | NS | 0.51 | 1.41 |
| hsa-miR-342 | 2.81 | 1.17 | NS |
| hsa-miR-345 | NS | 0.75 | 1.69 |
| hsa-miR-34a | 4.88 | 11.76 | 6.37 |
| hsa-miR-365 | 0.51 | NS | 0.87 |
| hsa-miR-375 | 19.18 | NS | NS |
| hsa-miR-378 | NS | 5.77 | 10.22 |
| hsa-miR-422a | 11.67 | 7.66 | NS |
| hsa-miR-422b | 1.88 | 4.9 | 1.29 |
| hsa-miR-425 | NS | NS | 4.28 |
| hsa-miR-7 | NS | NS | 1.9 |
| hsa-miR-92 | 0.72 | NS | 0.79 |
| hsa-miR-93 | 1.57 | NS | NS |
| hsa-miR-99b | NS | 0.7 | NS |

miRNAs upregulated in CD44+ cells

| | | | |
|---|---|---|---|
| hsa-miR-100 | NS | NS | −6.37 |
| hsa-miR-124a | NS | −0.61 | −1.27 |
| hsa-miR-125b | NS | −0.61 | −1.27 |
| hsa-miR-133a | NS | −9.49 | −1.27 |
| hsa-miR-142-3p | NS | NS | −3.9 |
| hsa-miR-181a | NS | −0.61 | −1.27 |
| hsa-miR-181d | NS | −0.61 | −1.27 |
| hsa-miR-19a | NS | −2.63 | −0.71 |
| hsa-miR-205 | NS | −0.61 | −11.42 |
| hsa-miR-22 | −1.85 | NS | NS |
| hsa-miR-29a | NS | NS | −0.92 |
| hsa-miR-29c | NS | NS | −0.79 |
| hsa-miR-301 | −1.09 | −0.76 | −0.76 |
| hsa-miR-328 | −1.76 | NS | NS |
| hsa-miR-33 | NS | −7.61 | −1.27 |
| hsa-miR-338 | NS | −0.61 | −1.27 |
| hsa-miR-374 | NS | NS | −2.99 |
| hsa-miR-451 | NS | −0.61 | −14 |
| hsa-miR-452 | NS | −2.9 | −11.28 |
| hsa-miR-501 | NS | NS | −0.84 |

Positive values indicate that miRNAs are expressed at reduced levels in CD44(+) cells compared to expression in the CD44(−) prostate tumor cell population. Negative values indicate that miRNAs are expressed at increased levels in CSCs. Expression changes <0.5 were deemed non-significant (NS).

TABLE 2
miRNAs differentially expressed in prostate cancer stem
cells (CSCs) purified from human prostate xenograft tumor
of LAPC4 using antibodies against the CD133 surface antigen,
representing prostate cancer stem cells.
miRs downregulated in CD133(+) cells

| | Relative Expression (log2) |
|---|---|
| miRNA | LAPC4 |
| hsa-let-7i | 0.5564 |
| hsa-miR-124a | 1.3196 |
| hsa-miR-126 | 2.2628 |
| hsa-miR-133a | 5.45 |
| hsa-miR-152 | 0.6367 |
| hsa-miR-15a | 1.333 |
| hsa-miR-196b | 0.5323 |
| hsa-miR-200a | 0.8372 |
| hsa-miR-22 | 0.7699 |
| hsa-miR-320 | 0.8032 |
| hsa-miR-335 | 13.6781 |
| hsa-miR-340 | 0.7567 |
| hsa-miR-34a | 6.1466 |
| hsa-miR-362 | 0.7987 |
| hsa-miR-365 | 18.7994 |
| hsa-miR-375 | 0.6335 |
| hsa-miR-425 | 0.8198 |
| hsa-miR-500 | 12.5725 |
| hsa-miR-92 | 0.5556 |
| hsa-miR-93 | 0.6517 |
| hsa-miR-98 | 0.5612 |

miRs upregulated in CD133(+) cells

| | Relative Expression (log2) |
|---|---|
| | LAPC4 |
| hsa-let-7c | −0.5581 |
| hsa-let-7e | −0.5223 |
| hsa-miR-101 | −0.7509 |
| hsa-miR-105 | −1.7903 |
| hsa-miR-10a | −0.5333 |
| hsa-miR-10b | −0.5592 |
| hsa-miR-125a | −0.7016 |
| hsa-miR-125b | −6.945 |
| hsa-miR-127 | −1.0802 |
| hsa-miR-133b | −1.848 |
| hsa-miR-140 | −1.2409 |
| hsa-miR-142-3p | −6.4863 |
| hsa-miR-142-5p | −8.6407 |
| hsa-miR-145 | −2.5335 |
| hsa-miR-146a | −6.3199 |
| hsa-miR-146b | −3.069 |
| hsa-miR-147 | −6.945 |
| hsa-miR-148a | −0.9964 |
| hsa-miR-148b | −0.5955 |
| hsa-miR-15b | −0.5743 |
| hsa-miR-16 | −0.6147 |
| hsa-miR-181a | −6.945 |
| hsa-miR-181d | −6.945 |
| hsa-miR-183 | −0.5309 |
| hsa-miR-18a | −1.8224 |
| hsa-miR-190 | −2.0407 |
| hsa-miR-192 | −0.8115 |
| hsa-miR-193b | −0.6514 |
| hsa-miR-195 | −0.5163 |
| hsa-miR-19a | −0.6698 |
| hsa-miR-19b | −0.7572 |
| hsa-miR-203 | −0.6154 |
| hsa-miR-205 | −0.5496 |
| hsa-miR-21 | −26.4581 |
| hsa-miR-214 | −0.9359 |
| hsa-miR-218 | −2.3908 |
| hsa-miR-221 | −1.4197 |
| hsa-miR-222 | −1.8574 |
| hsa-miR-223 | −6.9422 |
| hsa-miR-24 | −0.7757 |
| hsa-miR-25 | −0.6531 |
| hsa-miR-26b | −0.5395 |
| hsa-miR-27a | −1.9827 |
| hsa-miR-27b | −0.6736 |
| hsa-miR-28 | −0.9031 |
| hsa-miR-29a | −2.8784 |
| hsa-miR-29b | −2.0327 |
| hsa-miR-29c | −1.2433 |
| Has-miR-30a-3p | −1.1645 |
| Has-miR-30a-5p | −0.9717 |
| hsa-miR-30b | −0.5051 |
| hsa-miR-30c | −1 |
| hsa-miR-30d | −0.8061 |
| Has-miR-30e-5p | −1.455 |
| hsa-miR-32 | −0.7289 |
| Has-miR-324-3p | −0.638 |
| Has-miR-324-5p | −1.2068 |
| hsa-miR-328 | −3.0956 |
| hsa-miR-33 | −2.1178 |
| hsa-miR-331 | −0.7065 |
| hsa-miR-338 | −6.945 |
| hsa-miR-339 | −2.0332 |
| hsa-miR-345 | −0.7191 |
| hsa-miR-361 | −1.1723 |
| hsa-miR-374 | −1.108 |
| hsa-miR-378 | −1.2293 |
| hsa-miR-422a | −0.904 |
| hsa-miR-422b | −0.5609 |
| hsa-miR-423 | −2.138 |
| hsa-miR-433 | −2.2554 |
| hsa-miR-451 | −6.4959 |
| hsa-miR-452 | −6.945 |
| hsa-miR-501 | −0.5931 |
| hsa-miR-9 | −1.9282 |
| hsa-miR-96 | −1.7889 |

Positive values indicate that miRNAs are expressed at reduced levels in CD133(+) cells compared to expression in the CD133(−) prostate tumor cell population. Negative values indicate that miRNAs are expressed at increased levels in CSCs. Expression changes <0.5 were deemed non-significant and were not included in the table.

The differentially expressed miRNAs in Tables 1 and 2 represent targets for therapeutic intervention in prostate cancer. In particular, miRNAs with reduced expression in CSCs may be playing a tumor-suppressive role and can be used in ‘replacement therapy’ to retard tumor growth. miRNAs that are upregulated could be playing an oncogenic role and therefore can be silenced to eliminate cancer stem cells and the disease.

### Example 2

### miRNAs Differentially Expressed in Cancer Stem Cells Isolated from Side Population-Enriched Prostate Cancer Cells

The inventors also identified miRNAs that are differentially regulated in prostate CSCs, purified as a side population (SP) by flow cytometry. CSCs preferentially express high levels of a family of multi-drug resistance (MDR) proteins known as ABC transporters (Zhou et al., 2001; Lechner et al., 2002). High expression of ABC transporters enables efficient efflux of cytotoxic agents from the cell, thereby conferring chemoresistance to CSCs (Doyle and Ross, 2003). The Hoechst 33342 fluorescent dye is efficiently exported from CSCs by the ABC transporter complex, enabling identification and separation of CSCs. SP prostate cancer cells are a fraction of prostate cancer cells, highly enriched for CSCs, that are detected by dual-wavelength flow cytometry based on their ability to efflux fluorescent Hoechst 33342 dye (Patrawala et al., 2005; Hirschmann-Jax et al., 2004).

LAPC9 tumor xenografts were established in mice, and tumors were dissociated as described above in Example 1. Dissociated LAPC9 tumor cells were incubated for 2 hr at 37° C. in culture medium containing 5 μg/ml of Hoechst 33342 dye (cat. no. B2261; Sigma-Aldrich Co.; St. Louis, Mo., USA). Cells were analyzed for fluorescence using a Beckman-Coulter EPICS Elite fluorescence activated cell-sorter. Hoechst 33342 was excited with a UV laser at 351 to 364 nm, and its fluorescence was measured with a 515 nm SP filter (Hoechst blue) and a 608 nm EFLP optical filter (Hoechst red). CSCs expressing high levels of the ABC transporter proteins exported fluorescent dye and appeared on the ‘side’ of the FACS profile as a distinct Hoechst-low fraction. This side population was purified and was previously shown to be highly enriched for CSCs in LAPC9 tumors (Patrawala et al., 2005). The SP and the non-SP cell fractions were harvested and frozen immediately upon sorting. RNA was isolated as described above in Example 1.

Expression levels of 137 miRNAs were determined in duplicate by quantitative real-time PCR (as described above in Example 1) in CSCs enriched in the LAPC9 SP. Raw Ct values from CSCs were normalized to miR-24, a house-keeping miRNA, and expressed relative to the ones generated in the non-SP LAPC9 prostate cancer cells. miRNAs differentially expressed between the two populations are shown in Table 3.

TABLE 3
miRNAs differentially expressed in prostate cancer stem cells
enriched as a side population (SP) of LAPC9 tumor cells.

| | |
|---|---|
| | Relative expression (log₂) |
| miRNA | LAPC9 |

miRNAs downregulated in SP cells

| | |
|---|---|
| Has-let-7c | 1.91743 |
| hsa-miR-103 | 10.258164 |
| hsa-miR-10b | 8.1560155 |
| hsa-miR-126 | 3.0722085 |
| Has-miR-146a | 6.823249 |
| Has-miR-146b | 6.153386 |
| hsa-miR-15a | 10.643113 |
| hsa-miR-15b | 12.1120955 |
| hsa-miR-182 | 2.563866 |
| hsa-miR-183 | 5.2617965 |
| hsa-miR-191 | 1.1364705 |
| hsa-miR-195 | 2.9771485 |
| hsa-miR-200a | 1.5385825 |
| hsa-miR-200c | 0.939588 |
| hsa-miR-203 | 0.87379 |
| hsa-miR-205 | 9.0347945 |
| hsa-miR-21 | 0.5807815 |
| hsa-miR-218 | 1.0871205 |
| hsa-miR-27a | 1.095363 |
| hsa-miR-29b | 2.434234 |
| hsa-miR-29c | 0.7804065 |
| hsa-miR-30a-3p | 1.0177615 |
| hsa-miR-30a-5p | 2.217874 |
| hsa-miR-328 | 9.5805905 |
| hsa-miR-335 | 10.700541 |
| hsa-miR-365 | 0.9593445 |
| hsa-miR-375 | 1.3379015 |
| hsa-miR-422a | 3.120847 |
| hsa-miR-429 | 0.608145 |
| hsa-miR-9* | 1.2157095 |

miRNAs upregulated in SP cells

| | |
|---|---|
| Has-let-7b | −1.0670255 |
| hsa-miR-133b | −1.379272 |
| hsa-miR-142-3p | −0.510159 |
| hsa-miR-196a | −1.835175 |
| hsa-miR-196b | −1.514786 |
| hsa-miR-221 | −0.7282025 |
| hsa-miR-222 | −1.017886 |
| hsa-miR-223 | −1.3750955 |
| hsa-miR-320 | −1.8956015 |
| hsa-miR-33 | −0.752666 |
| hsa-miR-378 | −1.941396 |
| hsa-miR-425 | −1.4846465 |
| hsa-miR-451 | −7.5747555 |
| hsa-miR-452 | −3.483306 |
| hsa-miR-500 | −5.137463 |

Positive values indicate that miRNAs are expressed at reduced levels in the SP CSCs compared to expression in non-SP LAPC9 cells. Negative values indicate that miRNAs are expressed at increased levels in SP CSCs.

TABLE 4
miRNAs commonly deregulated in at least two fractions of prostate
cancer stem cells marked by either the CD44+ epitope, the
CD133+ epitope, or enriched in the side population (SP).

miRNA
miRNAs downregulated in CD44+ and CD133+ cells
hsa-miR-126
hsa-miR-335
hsa-miR-340
hsa-miR-34a
hsa-miR-365
hsa-miR-375
hsa-miR-425
hsa-miR-92
hsa-miR-93
miRNAs upregulated in CD44+ and CD133+ cells
hsa-miR-125b
hsa-miR-142-3p
hsa-miR-181a
hsa-miR-181d
hsa-miR-205
hsa-miR-29a
hsa-miR-29c
hsa-miR-328
hsa-miR-33
hsa-miR-338
hsa-miR-374
hsa-miR-451
hsa-miR-452
hsa-miR-501
miRNAs downregulated in CD44+ and SP cells
hsa-let-7c
hsa-miR-126
hsa-miR-15b
hsa-miR-182
hsa-miR-183
hsa-miR-191
hsa-miR-200c
hsa-miR-203
hsa-miR-218
hsa-miR-27a
hsa-miR-29b
hsa-miR-30a-3p
hsa-miR-30a-5p
hsa-miR-335
hsa-miR-365
hsa-miR-375
hsa-miR-422a
miRNAs upregulated in CD44+ and SP cells
hsa-miR-142-3p
hsa-miR-33
hsa-miR-451
hsa-miR-452
miRs downregulated in CD133+ and SP cells
hsa-miR-126
hsa-miR-15a
hsa-miR-200a
hsa-miR-335
hsa-miR-365
hsa-miR-375
miRs upregulated in CD133+ and SP cells
hsa-miR-133b
hsa-miR-142-3p
hsa-miR-221
hsa-miR-222
hsa-miR-223
hsa-miR-33
hsa-miR-378
hsa-miR-451
hsa-miR-452

TABLE 5
miRNAs commonly deregulated in prostate cancer
stem cells marked by the CD44+ and CD133+
epitopes, and enriched in the side population (SP).

miRNA
miRNAs downregulated CD44+/CD133+/SP cells
hsa-miR-126
hsa-miR-335
hsa-miR-365
hsa-miR-375
miRNAs upregulated CD44+/CD133+/SP cells
hsa-miR-142-3p
hsa-miR-33
hsa-miR-451
hsa-miR-452

The miRNAs in Table 3 represent targets for therapeutic intervention in prostate cancer. Importantly, functional manipulation of these miRNAs may sensitize the CSCs to chemotherapeutic agents effectively preventing recurrence.

Cells selected by the CD44 and CD133 epitopes, or enriched in the side population represent cancer stem cells (Tables 1, 2 and 3). Therefore, miRNAs differentially expressed in each of these cell populations are likely to indicate and/or contribute to the properties of cancer stem cells. miRNAs listed in Tables 1, 2 and 3 represent useful candidates for therapeutic intervention in the treatment of prostate cancer or cancer stem cells in general.

Table 4 shows miRNAs that are commonly deregulated in either CD44+/CD133+, CD44+/SP, or CD133+/SP cells. Table 5 shows miRNAs that are commonly deregulated in CD44+/CD133+/SP cells. Since these miRNAs represent a unique signature among various cancer stem cell populations, the miRNAs listed in Tables 4 and 5 represent preferred therapeutic targets or agents. miRNAs upregulated are ideal candidates to develop miRNA inhibitors in an effort to treat prostate cancer or cancer stem cells in general; miRNAs downregulated are ideal candidates for “miRNA replacement therapy” to treat prostate cancer or cancer stem cells in general.

### Example 3

### miRNAs Predicted to Regulate Cancer Stem Cell Genes

Common genetic regulatory mechanisms govern the unique properties of CSCs from different tumor types. For example, several types of CSCs, including multiple myeloma CSCs (Peacock et al., 2007), leukemia CSCs (Lessard and Sauvageau, 2003), and pancreatic CSCs (Li et al., 2007), are known to require activation of Sonic Hedgehog for survival. To further elucidate the elements regulating CSCs, the inventors compiled a list of CSC-related genes reported in the literature, and predicted whether transcripts of these genes were targeted by miRNAs. For this purpose, the 3′UTRs of the CSC-related genes were analyzed for regions of perfect complementarity with the seed sequences (nt 2-8 at the 5′ end) of over 800 miRNAs listed in mirBase (Release 12.0, September 2008) (Griffiths-Jones et al., 2008). Gene targets for the 800 miRNAs were also predicted from the list of CSC-related genes using the proprietary algorithm miRNATarget (Asuragen Inc. Austin, Tex., USA), and the combined results are displayed in Table 5. CSC genes included in this analysis are NANOG (Ben-Porath et al., 2008; Gu et al., 2007), POUF51 (also known as Oct-3/4) (Ben Porath et al., 2008; Gu et al., 2007), GLI1 (Liu et al., 2006; Clement et al., 2007), SMO (smoothened) (Karhadkar et al., 2004), PTCH-1/PTCH (patched) (Liu et al., 2006; Yang et al., 2008), WNT3A (Willert et al., 2003; Lu et al., 2006; Li et al., 2008; Dai et al., 2008), CTNNB1 (beta-catenin) (Jamieson et al., 2004; Malanchi et al., 2008), NOTCH1 (Fan et al., 2006), JAG1 (jagged) (Leong & Gao, 2008), SOX2 (Ben Porath et al., 2008; Gu et al., 2007), PROM1 (CD133) (Shepherd et al., 2008; Miki et al., 2007; Liu et al., 2006; Collins et al., 2005; Singh et al., 2003; Hermann et al., 2007), CD44 (Patrawala et al., 2006; Patrawala et al., 2007; Hurt et al., 2008; Al-Haji et al., 2003; Collins et al., 2005; Zhang et al., 2008; Gu et al., 2007), BMI1/PCFG4 (Liu et al., 2006; Lessard et al., 2003; Hurt et al., 2008), ABCG2/BCRP (Zhou et al., 2001; Lechner et al., 2002; Patrawala et al., 2005; Hirschmann-Jax et al., 2004; Dontu et al., 2003), ABCB1 (Bourguignon et al., 2008; Ho et al., 2008; Keshet et al., 2008; Lechner et al., 2002), ITGA2 (Collins et al., 2005; Patrawala et al., 2007), ITGB1 (Collins et al., 2005; Patrawala et al., 2007), PTEN (Hambardzumuyan et al., 2008; Wang et al., 2006; Zhou et al., 2007), STAT3 (Zhou et al., 2007), SOX4 (Sinner et al., 2007; Vermeulen et al., 2008), ALDH1A (Ginestier et al., 2007; Dylla et al., 2008), PSCA (prostate stem cell antigen) (Watabe et al., 2002; Birnie et al., 2008), ITGAV (integrin alpha V) (Birnie et al., 2008), NFKB1 (Birnie et al., 2008), BCL2 (Liu et al., 2006; Wang et al., 2006), CXCR4 (Miki et al., 2007; Liu et al., 2006; Hermann et al., 2007), PAPPA (Birnie et al., 2008), TJP2 (tight junction protein 2) (Birnie et al., 2008), ABI1 (Abl-interactor 1) (Birnie et al., 2008), BTG1 (Birnie et al., 2008), IL6 (Birnie et al., 2008), CFLAR (Birnie et al., 2008), SMU1 (Birnie et al., 2008), S100A3 (S 100 calcium binding protein A3) (Birnie et al., 2008), RASEF (Ras and EF-hand domain containing) (Birnie et al., 2008), IFNGR1 (interferon gamma receptor 1) (Birnie et al., 2008), IGFL1 (Birnie et al., 2008), MSMB (Birnie et al., 2008), CEACAM5 (Birnie et al., 2008), S100A7 (S100 calcium binding protein A7) (Birnie et al., 2008), HPGD (Birnie et al., 2008), CEACAM7 (Birnie et al., 2008), TFF1 (trefoil factor 1) (Birnie et al., 2008), PIP (prolactin-induced protein) (Birnie et al., 2008), Secretagogin (SCGN) (Liu et al., 2007), SWAP70 (Liu et al., 2007), Cyclin Y (CCYN) (Liu et al., 2007), C10orf9 (Liu et al., 2007), cell division cycle 123 homolog (CDC123, C10orf7) (Liu et al., 2007), alkylation repair homolog 1 (ALKBH1) (Liu et al., 2007), transducer of ERBB2 (TOB2) (Liu et al., 2007), xenotropic and polytropic retrovirus receptor (XPR1) (Liu et al., 2007), CD59 molecule (CD59) (Liu et al., 2007), low density lipoprotein-related protein 2 (LRP2) (Liu et al., 2007), proteolipid protein 2 (PLP2) (Liu et al., 2007), mitogen-activated protein kinase 14 (MAPK14) (Liu et al., 2007), matrix metallopeptidase 7 (MMPI) (Liu et al., 2007), matrix Gla protein (MGP) (Liu et al., 2007), myeloid leukemia factor 1 (MLF1) (Liu et al., 2007), filaminB beta (actin binding protein 278) (FLNB) (Liu et al., 2007), sodium channel modifier 1 (SCNM1) (Liu et al., 2007), cornichon homolog 4 (CNIH4 also known as HSPC163) (Liu et al., 2007), receptor accessory protein 5 (REEP5) (Liu et al., 2007), solute carrier family 25, member 33 (SLC25A33) (Liu et al., 2007), solute carrier family 44, member 1 (SLC44A1) (Liu et al., 2007), transmembrane channel-like 4 (TMC4) (Liu et al., 2007), zinc finger DHHC-type containing 2 (ZDHHC2) (Liu et al., 2007), toll-like receptor adaptor molecule 2 (TICAM2) (Liu et al., 2007), KDEL (Lys-Asp-Glu-Leu) endoplasmic reticulum protein retention receptor 3 (KDELR3) (Liu et al., 2007), glucosamine-6-phosphate deaminase 1 (GNPDA1) (Liu et al., 2007), thioesterase superfamily member 2 (THEM2) (Liu et al., 2007), debranching enzyme homolog 1 (DBR1) (Liu et al., 2007), solute carrier family 38, member 9 (SLC38A9) (Liu et al., 2007), N-acetyltrans-ferase 10 (GCN5-related) (NAT10) (Liu et al., 2007), chromosome 16 open reading frame 33 (C16orf33) (Liu et al., 2007), lactate dehydrogenase A (LDHA) (Liu et al., 2007), major histocompatibility complex, class I-related (MR1) (Liu et al., 2007), leucyl-tRNA synthetase (LARS) (Liu et al., 2007), GTP binding protein 1 (GTPBP1) (Liu et al., 2007), protease serine 16 (PRSS16) (Liu et al., 2007), WAP four-disulfide core domain 2 (WFDC2) (Liu et al., 2007), aurora kinase B (AURKB) (Liu et al., 2007), 3-hydroxybutyrate dehydrogenase, type 2 (BDH2) (Liu et al., 2007), dehydrogenase/reductase (SDR family) member 4 (DHRS4) (Liu et al., 2007), abhydrolase domain containing 14B (ABHD14B) (Liu et al., 2007), EF-hand calcium binding domain 4A (EFCAB4A) (Liu et al., 2007), enoyl coenzyme A hydratase domain containing 2 (ECHDC2) (Liu et al., 2007), golgi autoantigen, golgin subfamily a,*B (GOLGA8B) (Liu et al., 2007), ATPase type 13A3 (ATP13A3) (Liu et al., 2007), prolyl endopeptidase-like (PREPL) (Liu et al., 2007), cytochrome P450, family 4, subfamily V, polypeptide 2 (CYP4V2) (Liu et al., 2007), JTV1 (Liu et al., 2007), isoprenyl-cysteine carboxyl methyltransferase (ICMT) (Liu et al., 2007), DNA (cytosine-5-)-methyltransferase 3 alpha (DNMT3A) (Liu et al., 2007), histamine N-methyltransferase (HNMT) (Liu et al., 2007), methyltransferase like 7A (METTL7A) (Liu et al., 2007), methyltransferase like 2A (METTL2A) (Liu et al., 2007), methyltransferase like 2B (METTL2B) (Liu et al., 2007), ezrin (EZR) (Liu et al., 2007), tumor protein D52 (TPD52) (Liu et al., 2007), actin related protein 2/3 complex, subunit 5 16 kDa (ARPC5) (Liu et al., 2007), nucleolar protein 8 (NOL8) (Liu et al., 2007), N-ehylmaleimide-sensitive factor (NSF) (Liu et al., 2007), RAD23 homolog B (RAD23B) (Liu et al., 2007), signal recognition particle 54 kDa (SRP54) (Liu et al., 2007), heat shock 70kDa protein 2 (HSPA2) (Liu et al., 2007), mediator complex subunit 1 (MED 1) (Liu et al., 2007), THAP domain containing, apoptosis associated protein 2 (THAP2) (Liu et al., 2007), cold inducible RNA binding protein (CIRBP) (Liu et al., 2007), small nuclear ribonucleoprotein polypeptide N (SNRPN) (Liu et al., 2007), superkiller viralicidic activity 2-like 2 (SKIV2L2) (Liu et al., 2007), dual specificity phosphatase 10 (DUSP10) (Liu et al., 2007), signal sequence receptor, alpha (SSR1) (Liu et al., 2007), v-erb-a erythroblastic leukemia viral oncogene homolog 4 (ERBB4) (Liu et al., 2007), epithelial membrane protein 1 (EMP1) (Liu et al., 2007), low density lipoprotein receptor-related protein associated protein 1 (LRPAP1) (Liu et al., 2007), SCY1-like binding protein 1 (SCYL1BP1) (Liu et al., 2007), cleavage stimulation factor, 3′ pre-RNA, subunit 1, 50 kDa (CSTF1) (Liu et al., 2007), Kelch-like 20 (KLHL20) (Liu et al., 2007), DnaJ (Hsp40) homolog, subfamily C, member 13 (DNAJC13) (Liu et al., 2007), amyloid beta (A4) precursor-like protein 2 (APLP2) (Liu et al., 2007), sorbin and SH3 domain containing 2 (SORBS2) (Liu et al., 2007), DnaJ (Hsp40) homolog, subfamily B, member 1 (DNAJB1) (Liu et al., 2007), nebulette (NEBL) (Liu et al., 2007), SH3 domain binding glutamic acid-rich protein like (SH3BGRL) (Liu et al., 2007), nudix (nucleoside diphosphate linked moiety X)-type motif 5 (NUDT5) (Liu et al., 2007), GABA(A) receptor-associated protein like 1 (GABARAPL1) (Liu et al., 2007), microtubule-associated protein tau (MAPT) (Liu et al., 2007), discoidin, CUB and LCCL domain containing 1 (DCBLD1) (Liu et al., 2007), serine threonine kinase 39 (STK39) (Liu et al., 2007), protein kinase N2 (PKN2) (Liu et al., 2007), casein kinase 2, alpha 1 polypeptide (CSNK2A1) (Liu et al., 2007), paired immunoglobin-like type 2 receptor beta (PILRB) (Liu et al., 2007), endoplasmic reticulum to nucleus signaling 1 (ERN1) (Liu et al., 2007), serum/gluco-corticoid regulated kinase family, member 3 (SGK3) (Liu et al., 2007), WEE1 homolog (WEE1) (Liu et al., 2007), microtubule associated serine/threonine kinase family member 4 (MAST4) (Liu et al., 2007), chromosome 11 open reading frame 17 (C11orf17) (Liu et al., 2007), nucleoproin 37 kDa (NUP37) (Liu et al., 2007), clathrin, heavy chain (Hc) (CLTC) (Liu et al., 2007), coatomer protein complex, subunit beta 2 (beta prime) (COPB2) (Liu et al., 2007), solute carrier family 25 (mitochondrial carrier; phosphate carrier) member 25 (SLC25A25) (Liu et al., 2007), EGFR-coamplified and overexpressed protein (ECOP) (Liu et al., 2007), phosphodi-esterase 8A (PDE8A) (Liu et al., 2007), signal tranducing adaptor molecule (SH3 domain and ITAM motif) 1 (STAM) (Liu et al., 2007), tubulin, beta (TUBB) (Liu et al., 2007), sorting nexin 6 (SNX6) (Liu et al., 2007), RAB23, member RAS oncogene family (RAB23) (Liu et al., 2007), phospholipase A2-activating protein (PLAA) (Liu et al., 2007), stanniocalcin 2 (STC2) (Liu et al., 2007), lactotransferrin (LTF) (Liu et al., 2007), interferon regulator factor 9 (IRF9) (Liu et al., 2007), ataxin 3 (ATXN3) (Liu et al., 2007), general transcription factor IIIC, polypeptide 3, 102 kDa (GTF3C3) (Liu et al., 2007), glycogen synthase kinase 3 beta (GSK3B) (Liu et al., 2007), kruppel-like factor 10 (KLF10) (Liu et al., 2007), elongation factor, RNA polymerase II,2 (ELL2) (Liu et al., 2007), zinc finger and BTB domain containing 20 (ZBTB20) (Liu et al., 2007), iroquois homeobox 3 (IRX3) (Liu et al., 2007), v-ets erythroblastosis virus E26 oncogene homolog 1 (ETS1) (Liu et al., 2007), SERTA domain containing 1 (SERTAD1) (Liu et al., 2007), transmembrane protein 101 (TMEM101) (Liu et al., 2007), v-maf musculo-aponeurotic fibrosarcoma oncogene homolog F (MAFF) (Liu et al., 2007), splicing factor proline/glutamine-rich (polypyrimidine tract binding protein associated) (SFPQ) (Liu et al., 2007), Cbp/p300-interacting transactivator, with Glu/Asp-rich carboxy-terminal domain, 4 (CITED4) (Liu et al., 2007), CCAAT/enhancer binding protein (C/EBP), delta (CEBPD) (Liu et al., 2007), eukaryotic translation initiation factor 4E family member 2 (EIF4E2) (Liu et al., 2007), heparan sulfate 2-O-sulfotransferase 1 (HS2ST1) (Liu et al., 2007), alkylglycerone phosphate synthase (AGPS) (Liu et al., 2007), phospho-glycerate kinase 1 (PGK1) (Liu et al., 2007), 5-aminoimidazole-4-carboxamide ribonucleotide formyltrans-ferase/IMP cyclohydrolase (ATIC) (Liu et al., 2007), ethanolamine kinase 1 (ETNK1) (Liu et al., 2007), asparagine-linked glycosylation 2 homolog (ALG2) (Liu et al., 2007), ubiquitin-conjugating enzyme E2F (UBE2F) (Liu et al., 2007), membrane-associated ring finger (C3HC4) 8 (MARCH8) (Liu et al., 2007), CCR4-NOT transcription complex, subunit 4 (CNOT4) (Liu et al., 2007), ring finger protein 8 (RNF8) (Liu et al., 2007), proteasome (prosome, macropain) subunit, alpha type, 5 (PSMA5) (Liu et al., 2007), alport syndrome, mental retardation, midface hypoplasia and elliptocytosis chromosomal region gene 1 (AMMERCR1) (Liu et al., 2007), integrator complex subunit 2 (1NTS2) (Liu et al., 2007), family with sequence similarity 152, member A (FAM152A) (Liu et al., 2007), integrator complex subunit 8 (INTS8) (Liu et al., 2007), THUMP domain containing 3 (THUMPD3) (Liu et al., 2007), centrosomal protein 350 kDa (CEP350) (Liu et al., 2007), Ewing tumor-associated antigen 1 (ETAA1) (Liu et al., 2007), WD repeat domain 68 (WDR68) (Liu et al., 2007), viral DNA polymerase-transactivated protein 6 (DNAPTP6) (Liu et al., 2007), chromosome 7 open reading frame 25 (C7orf25) (Liu et al., 2007), chromosome 2 open reading frame 60 (C2orf60) (Liu et al., 2007), chromosome 17 open reading frame 71 (C17orf71) (Liu et al., 2007), chromosome 7 open reading frame 36 (C7orf36) (Liu et al., 2007), elongation protein 4 homolog (ELP4) (Liu et al., 2007), nudE nuclear distribution gene E homolog (A. nidulans)-like 1 (NDEL1) (Liu et al., 2007), chromosome 1 open reading frame 63 (C1orf63) (Liu et al., 2007), family with sequence similarity 172, member A (FAM172A) (Liu et al., 2007), family with sequence similarity 53, member C (FAM53C) (Liu et al., 2007), immediate early response 5 (IER5) (Liu et al., 2007), KIAA0146 (Liu et al., 2007), transmembrane protein 63A (TMEM63A) (Liu et al., 2007), NEDD4 binding protein 2-like 1 (N4BP2L1) (Liu et al., 2007), LY6/PLAUR domain containing 6B (LYPD6B) (Liu et al., 2007), BEX family member 5 (BEX5) (Liu et al., 2007), KIAAl217 (Liu et al., 2007), chromosome 4 open reading frame 7 (C4orf7) (Liu et al., 2007), chromosome 9 open reading frame 64 (C9orf64) (Liu et al., 2007), Zyg-11 homolog B (ZYG11B) (Liu et al., 2007), family with sequence similarity 160, member B1 (FAM160B1) (Liu et al., 2007), limb region 1 homolog (LMBR1) (Liu et al., 2007), nuclear casein kinase and cyclin-dependent kinase substrate 1 (NUCKS1) (Liu et al., 2007), ankyrin repeat domain 13C (ANKRD13C) (Liu et al., 2007), coiled-coil domain containing 43 (CCDC43) (Liu et al., 2007), UHRF1 binding protein 1 (UHRF1BP1) (Liu et al., 2007), chromosome 1 open reading frame 163 (C1orf163) (Liu et al., 2007), axin interactor, dorsalization associated (AIDA) (Liu et al., 2007), chromosome 4 open reading frame 32 (C4orf32) (Liu et al., 2007), NES (nestin) (Zhang et al., 2008; Gu et al., 2007; Lechner et al., 2002), c-kit (KIT, CD 117) (Zhang et al., 2008), GLI2 (Liu et al., 2006; Thiyagarajan et al., 2007), MCL1 (Konopleva et al., 2006), IL4 (Francipane et al., 2008), MUC1 (Engelmann et al., 2008), ABCA3 (Hirschmann-Jax et al., 2004), SHH (sonic hedgehog) (Peacock et al., 2007; Karhadkar et al., 2004; Vezina & Bushman 2007), IHH (Indian hedgehog) (Peacock et al., 2007; Karhadkar et al., 2004; Vezina & Bushman 2007).

CSC-related genes that contain predicted target sites for human miRNAs include, but are not limited to (listed as—Gene title, Gene symbol, Accession no., miRNA):

Nanog—NANOG—NM_{—}024865—hsa-miR-1207-5p, hsa-miR-1243, hsa-miR-1273, hsa-miR-128, hsa-miR-1283, hsa-miR-130b*, hsa-miR-1321, hsa-miR-150, hsa-miR-154, hsa-miR-1827, hsa-miR-198, hsa-miR-199a-5p, hsa-miR-199b-5p, hsa-miR-202*, hsa-miR-20b*, hsa-miR-29b-2*, hsa-miR-302a*, hsa-miR-30b*, hsa-miR-340*, hsa-miR-34a, hsa-miR-34c, hsa-miR-363*, hsa-miR-376a*, hsa-miR-432*, hsa-miR-485-5p, hsa-miR-508-5p, hsa-miR-511, hsa-miR-513b, hsa-miR-520d-5p, hsa-miR-524-5p, hsa-miR-526b, hsa-miR-541*, hsa-miR-545, hsa-miR-548c-3p, hsa-miR-548l, hsa-miR-548n, hsa-miR-548p, hsa-miR-551b*, hsa-miR-553, hsa-miR-566, hsa-miR-567, hsa-miR-575, hsa-miR-579, hsa-miR-589*, hsa-miR-605, hsa-miR-624, hsa-miR-643, hsa-miR-661, hsa-miR-664, hsa-miR-664*, hsa-miR-7-1*, hsa-miR-7-2*, hsa-miR-720, hsa-miR-765, hsa-miR-766, hsa-miR-769-5p, hsa-miR-886-3p, hsa-miR-889, and hsa-miR-940.

Oct-3/4—POUF51 variant 1/PouF51 variant 2—NM_{—}002701/NM_{—}203289—hsa-miR-1257, hsa-miR-221*, hsa-miR-299-3p, hsa-miR-324-3p, hsa-miR-335, hsa-miR-339-5p, hsa-miR-384, hsa-miR-412, hsa-miR-508-5p, hsa-miR-548d-3p, hsa-miR-551b*, hsa-miR-595, and hsa-miR-892a.

Gli1—GUI—NM_{—}005269—hsa-let-7f-2*, hsa-miR-1184, hsa-miR-1255a, hsa-miR-1255b, hsa-miR-1284, hsa-miR-1288, hsa-miR-1299, hsa-miR-1301, hsa-miR-34a, hsa-miR-34b, hsa-miR-34c, hsa-miR-361-3p, hsa-miR-373*, hsa-miR-374a*, hsa-miR-424*, hsa-miR-509-3-5p, hsa-miR-509-5p, hsa-miR-513a-3p, hsa-miR-616*, and hsa-miR-875-5p.

Smoothened—SMO—NM_{—}005631—hsa-let-7a*, hsa-let-7b*, hsa-let-7f-1*, hsa-miR-1184, hsa-miR-1185, hsa-miR-1205, hsa-miR-1207-3p, hsa-miR-1207-5p, hsa-miR-1227, hsa-miR-1252, hsa-miR-125a-5p, hsa-miR-125b, hsa-miR-1269, hsa-miR-1270, hsa-miR-1271, hsa-miR-127-5p, hsa-miR-1280, hsa-miR-1291, hsa-miR-1299, hsa-miR-1301, hsa-miR-130b*, hsa-miR-1322, hsa-miR-135a, hsa-miR-135b, hsa-miR-143, hsa-miR-146b-3p, hsa-miR-149*, hsa-miR-188-3p, hsa-miR-203, hsa-miR-218-1*, hsa-miR-30c-1*, hsa-miR-30c-2*, hsa-miR-31*, hsa-miR-324-3p, hsa-miR-326, hsa-miR-328, hsa-miR-330-5p, hsa-miR-338-3p, hsa-miR-339-5p, hsa-miR-370, hsa-miR-377*, hsa-miR-381, hsa-miR-423-3p, hsa-miR-425, hsa-miR-452*, hsa-miR-483-5p, hsa-miR-490-3p, hsa-miR-503, hsa-miR-504, hsa-miR-505, hsa-miR-508-5p, hsa-miR-516a-3p, hsa-miR-516b*, hsa-miR-518c*, hsa-miR-518d-5p, hsa-miR-518e*, hsa-miR-518f*, hsa-miR-519a*, hsa-miR-519b-5p, hsa-miR-519c-5p, hsa-miR-520c-5p, hsa-miR-522*, hsa-miR-523*, hsa-miR-526a, hsa-miR-548d-3p, hsa-miR-574-5p, hsa-miR-583, hsa-miR-588, hsa-miR-619, hsa-miR-620, hsa-miR-626, hsa-miR-629*, hsa-miR-645, hsa-miR-646, hsa-miR-657, hsa-miR-659, hsa-miR-661, hsa-miR-668, hsa-miR-7, hsa-miR-768-5p, hsa-miR-874, hsa-miR-877, hsa-miR-943, and hsa-miR-96.

Patched-1—PTCH-1/PTCH—NM_{—}000264—hsa-abi-13135, hsa-abi-13142, hsa-abi-13268, hsa-let-7a*, hsa-let-7b*, hsa-let-7f-1*, hsa-let-7f-2*, hsa-miR-100*, hsa-miR-101, hsa-miR-103, hsa-miR-107, hsa-miR-1178, hsa-miR-1179, hsa-miR-1182, hsa-miR-1183, hsa-miR-1202, hsa-miR-1203, hsa-miR-1205, hsa-miR-1208, hsa-miR-1225-5p, hsa-miR-1226, hsa-miR-1228, hsa-miR-124*, hsa-miR-1246, hsa-miR-1249, hsa-miR-1258, hsa-miR-125a-3p, hsa-miR-125a-5p, hsa-miR-125b, hsa-miR-126*, hsa-miR-1262, hsa-miR-1272, hsa-miR-1275, hsa-miR-127-5p, hsa-miR-1276, hsa-miR-1283, hsa-miR-1284, hsa-miR-1286, hsa-miR-1296, hsa-miR-1299, hsa-miR-1300, hsa-miR-1303, hsa-miR-130a*, hsa-miR-132, hsa-miR-1324, hsa-miR-135a*, hsa-miR-136, hsa-miR-138, hsa-miR-140-3p, hsa-miR-141, hsa-miR-143, hsa-miR-144, hsa-miR-146a*, hsa-miR-148a*, hsa-miR-148b*, hsa-miR-149*, hsa-miR-150, hsa-miR-151-5p, hsa-miR-153, hsa-miR-154*, hsa-miR-155*, hsa-miR-15a, hsa-miR-15b, hsa-miR-16, hsa-miR-16-2*, hsa-miR-182, hsa-miR-186, hsa-miR-188-3p, hsa-miR-18a, hsa-miR-18b, hsa-miR-192*, hsa-miR-193b*, hsa-miR-194, hsa-miR-194*, hsa-miR-195, hsa-miR-195*, hsa-miR-197, hsa-miR-199a*, hsa-miR-200a, hsa-miR-200b, hsa-miR-200c, hsa-miR-203, hsa-miR-204, hsa-miR-20a*, hsa-miR-211, hsa-miR-212, hsa-miR-214, hsa-miR-22, hsa-miR-220c, hsa-miR-221*, hsa-miR-23a, hsa-miR-23b, hsa-miR-24, hsa-miR-24-1*, hsa-miR-24-2*, hsa-miR-27a, hsa-miR-27b, hsa-miR-28-5p, hsa-miR-296-3p, hsa-miR-299-3p, hsa-miR-29a*, hsa-miR-300, hsa-miR-302c*, hsa-miR-30a*, hsa-miR-30c-1*, hsa-miR-30c-2*, hsa-miR-30d*, hsa-miR-30e*, hsa-miR-31*, hsa-miR-324-3p, hsa-miR-326, hsa-miR-329, hsa-miR-330-5p, hsa-miR-337-3p, hsa-miR-338-3p, hsa-miR-338-5p, hsa-miR-339-3p, hsa-miR-33a*, hsa-miR-342-3p, hsa-miR-345, hsa-miR-34a*, hsa-miR-362-3p, hsa-miR-369-3p, hsa-miR-373*, hsa-miR-377, hsa-miR-378*, hsa-miR-379*, hsa-miR-381, hsa-miR-410, hsa-miR-411*, hsa-miR-421, hsa-miR-423-5p, hsa-miR-424, hsa-miR-425, hsa-miR-429, hsa-miR-432*, hsa-miR-486-3p, hsa-miR-487a, hsa-miR-488, hsa-miR-493*, hsa-miR-495, hsa-miR-497, hsa-miR-498, hsa-miR-499-3p, hsa-miR-499-5p, hsa-miR-502-5p, hsa-miR-505*, hsa-miR-513b, hsa-miR-539, hsa-miR-541*, hsa-miR-542-3p, hsa-miR-543, hsa-miR-544, hsa-miR-545, hsa-miR-548a-3p, hsa-miR-548a-5p, hsa-miR-548b-5p, hsa-miR-548c-3p, hsa-miR-548c-5p, hsa-miR-548d-5p, hsa-miR-548e, hsa-miR-548f, hsa-miR-548g, hsa-miR-548h, hsa-miR-548i, hsa-miR-548j, hsa-miR-548n, hsa-miR-548p, hsa-miR-558, hsa-miR-559, hsa-miR-561, hsa-miR-564, hsa-miR-570, hsa-miR-576-5p, hsa-miR-578, hsa-miR-583, hsa-miR-587, hsa-miR-589, hsa-miR-591, hsa-miR-593, hsa-miR-593*, hsa-miR-597, hsa-miR-605, hsa-miR-608, hsa-miR-609, hsa-miR-615-5p, hsa-miR-616*, hsa-miR-624, hsa-miR-625*, hsa-miR-629*, hsa-miR-634, hsa-miR-637, hsa-miR-646, hsa-miR-651, hsa-miR-655, hsa-miR-656, hsa-miR-659, hsa-miR-665, hsa-miR-708, hsa-miR-767-3p, hsa-miR-767-5p, hsa-miR-768-3p, hsa-miR-769-5p, hsa-miR-770-5p, hsa-miR-802, hsa-miR-875-3p, hsa-miR-877, hsa-miR-885-5p, hsa-miR-888, hsa-miR-891b, hsa-miR-9, hsa-miR-922, hsa-miR-92a-2*, hsa-miR-939, and hsa-miR-944.

Wnt3A—WNT3A—NM_{—}033131—hsa-abi-4629, hsa-miR-100*, hsa-miR-103, hsa-miR-107, hsa-miR-1179, hsa-miR-1205, hsa-miR-1207-5p, hsa-miR-1225-3p, hsa-miR-1233, hsa-miR-1236, hsa-miR-1247, hsa-miR-1249, hsa-miR-1254, hsa-miR-1255a, hsa-miR-1255b, hsa-miR-1260, hsa-miR-1266, hsa-miR-127-3p, hsa-miR-1274a, hsa-miR-1278, hsa-miR-128, hsa-miR-1281, hsa-miR-1285, hsa-miR-1287, hsa-miR-1289, hsa-miR-1293, hsa-miR-1296, hsa-miR-1304, hsa-miR-1321, hsa-miR-138, hsa-miR-145*, hsa-miR-146b-3p, hsa-miR-148b*, hsa-miR-149*, hsa-miR-15a, hsa-miR-15b, hsa-miR-16, hsa-miR-1827, hsa-miR-185, hsa-miR-185*, hsa-miR-195, hsa-miR-214, hsa-miR-221*, hsa-miR-25*, hsa-miR-27a, hsa-miR-27b, hsa-miR-296-5p, hsa-miR-298, hsa-miR-30b*, hsa-miR-323-5p, hsa-miR-324-3p, hsa-miR-326, hsa-miR-330-5p, hsa-miR-331-3p, hsa-miR-339-5p, hsa-miR-361-3p, hsa-miR-363*, hsa-miR-410, hsa-miR-421, hsa-miR-423-5p, hsa-miR-424, hsa-miR-432, hsa-miR-432*, hsa-miR-483-3p, hsa-miR-483-5p, hsa-miR-485-5p, hsa-miR-491-5p, hsa-miR-497, hsa-miR-503, hsa-miR-513, hsa-miR-516b, hsa-miR-518a-5p, hsa-miR-518c*, hsa-miR-527, hsa-miR-541, hsa-miR-564, hsa-miR-600, hsa-miR-608, hsa-miR-612, hsa-miR-629*, hsa-miR-634, hsa-miR-637, hsa-miR-646, hsa-miR-654-5p, hsa-miR-658, hsa-miR-661, hsa-miR-663, hsa-miR-664, hsa-miR-671-3p, hsa-miR-744, hsa-miR-765, hsa-miR-874, hsa-miR-923, hsa-miR-92a-1*, hsa-miR-92a-2*, hsa-miR-92b*, hsa-miR-938, hsa-miR-939, and hsa-miR-940.

β-catenin—CTNNB1—NM_{—}001904—hsa-abi-11143, hsa-abi-371, hsa-abi-408, hsa-abi-4400, hsa-abi-693, hsa-abi-8027, hsa-let-7a*, hsa-let-7b*, hsa-let-7f-1*, hsa-miR-, 483, hsa-miR-, 589, hsa-miR-, 659, hsa-miR-1233, hsa-miR-1260, hsa-miR-1264, hsa-miR-1276, hsa-miR-138-1*, hsa-miR-139, hsa-miR-148a*, hsa-miR-148b*, hsa-miR-150, hsa-miR-16-1*, hsa-miR-1826, hsa-miR-183*, hsa-miR-200a, hsa-miR-214, hsa-miR-29a*, hsa-miR-302f, hsa-miR-320a, hsa-miR-320b, hsa-miR-320c, hsa-miR-320d, hsa-miR-330-3p, hsa-miR-33a*, hsa-miR-33b, hsa-miR-340, hsa-miR-381, hsa-miR-496, hsa-miR-497*, hsa-miR-512-5p, hsa-miR-526b, hsa-miR-532-3p, hsa-miR-543, hsa-miR-545, hsa-miR-548g, hsa-miR-548l, hsa-miR-548n, hsa-miR-561, hsa-miR-568, hsa-miR-578, hsa-miR-579, hsa-miR-581, hsa-miR-582-3p, hsa-miR-589*, hsa-miR-592, hsa-miR-618, hsa-miR-624, hsa-miR-624*, hsa-miR-640, hsa-miR-641, hsa-miR-657, hsa-miR-668, hsa-miR-768-3p, hsa-miR-885-5p, hsa-miR-892b, and hsa-miR-935.

Notch-1—NOTCH1—NM_{—}017617—hsa-miR-101, hsa-miR-1207-5p, hsa-miR-1227, hsa-miR-1228*, hsa-miR-1234, hsa-miR-1254, hsa-miR-125a-3p, hsa-miR-125a-5p, hsa-miR-125b, hsa-miR-1263, hsa-miR-1264, hsa-miR-1275, hsa-miR-1281, hsa-miR-1282, hsa-miR-1283, hsa-miR-1290, hsa-miR-1291, hsa-miR-1293, hsa-miR-1301, hsa-miR-1308, hsa-miR-1322, hsa-miR-138, hsa-miR-138-2*, hsa-miR-139, hsa-miR-139-5p, hsa-miR-148a, hsa-miR-148b, hsa-miR-149, hsa-miR-150, hsa-miR-152, hsa-miR-155*, hsa-miR-1825, hsa-miR-183, hsa-miR-18a*, hsa-miR-196a*, hsa-miR-200b, hsa-miR-200c, hsa-miR-221*, hsa-miR-28-5p, hsa-miR-30b, hsa-miR-30c, hsa-miR-331-3p, hsa-miR-335*, hsa-miR-34a, hsa-miR-34b, hsa-miR-34b*, hsa-miR-34c-5p, hsa-miR-369-3p, hsa-miR-421, hsa-miR-425, hsa-miR-429, hsa-miR-433, hsa-miR-449a, hsa-miR-449b, hsa-miR-455-3p, hsa-miR-455-5p, hsa-miR-485-5p, hsa-miR-488*, hsa-miR-490-3p, hsa-miR-509-3p, hsa-miR-511, hsa-miR-513b, hsa-miR-515-5p, hsa-miR-516b, hsa-miR-518c*, hsa-miR-519e*, hsa-miR-541, hsa-miR-545*, hsa-miR-548c-3p, hsa-miR-548k, hsa-miR-548n, hsa-miR-551b*, hsa-miR-561, hsa-miR-564, hsa-miR-568, hsa-miR-577, hsa-miR-582, hsa-miR-582-5p, hsa-miR-593*, hsa-miR-602, hsa-miR-603, hsa-miR-604, hsa-miR-608, hsa-miR-637, hsa-miR-643, hsa-miR-654-5p, hsa-miR-663b, hsa-miR-664, hsa-miR-665, hsa-miR-671-5p, hsa-miR-708, hsa-miR-760, hsa-miR-766, hsa-miR-874, hsa-miR-876-3p, hsa-miR-885-3p, hsa-miR-886-5p, hsa-miR-92, hsa-miR-92a-1*, hsa-miR-92b, and hsa-miR-940.

Jagged 1—JAG1—NM_{—}000214—hsa-abi-13097, hsa-abi-13196, hsa-abi-13248, hsa-abi-13259, hsa-abi-13261, hsa-abi-13268, hsa-abi-371, hsa-abi-4629, hsa-abi-6374, hsa-abi-6775, hsa-abi-9630, hsa-let-7a*, hsa-let-7a*, hsa-let-7b*, hsa-let-7b*, hsa-let-7f-1*, hsa-let-7f-1*, hsa-miR-1184, hsa-miR-1201, hsa-miR-1208, hsa-miR-124, hsa-miR-1245, hsa-miR-1246, hsa-miR-1253, hsa-miR-126, hsa-miR-126*, hsa-miR-1262, hsa-miR-1267, hsa-miR-128, hsa-miR-1282, hsa-miR-1284, hsa-miR-1284, hsa-miR-1285, hsa-miR-129, hsa-miR-1290, hsa-miR-129-5p, hsa-miR-129-5p, hsa-miR-1297, hsa-miR-1300, hsa-miR-1301, hsa-miR-130a*, hsa-miR-1323, hsa-miR-140-3p, hsa-miR-140-3p, hsa-miR-140-5p, hsa-miR-141, hsa-miR-142-5p, hsa-miR-145, hsa-miR-146a, hsa-miR-146b-5p, hsa-miR-148a*, hsa-miR-153, hsa-miR-16-1*hsa-abi-13242, hsa-miR-182, hsa-miR-185*, hsa-miR-186, hsa-miR-196a*, hsa-miR-199a-5p, hsa-miR-199b-5p, hsa-miR-200a, hsa-miR-200a*, hsa-miR-200b, hsa-miR-200b*, hsa-miR-200c, hsa-miR-21, hsa-miR-21, hsa-miR-214, hsa-miR-214*, hsa-miR-217, hsa-miR-218, hsa-miR-218-2*, hsa-miR-218-2*, hsa-miR-22*, hsa-miR-220c, hsa-miR-23a, hsa-miR-23b, hsa-miR-26a, hsa-miR-26b, hsa-miR-26b*, hsa-miR-297, hsa-miR-298, hsa-miR-300, hsa-miR-30a*, hsa-miR-30a*, hsa-miR-30d*, hsa-miR-30d*, hsa-miR-30e*, hsa-miR-30e*, hsa-miR-32*, hsa-miR-320, hsa-miR-335*, hsa-miR-345, hsa-miR-34a, hsa-miR-34b*, hsa-miR-34c-5p, hsa-miR-369-3p, hsa-miR-371-5p, hsa-miR-371-5p, hsa-miR-377, hsa-miR-381, hsa-miR-381, hsa-miR-410, hsa-miR-421, hsa-miR-424*, hsa-miR-425, hsa-miR-429, hsa-miR-432, hsa-miR-432*, hsa-miR-448, hsa-miR-449a, hsa-miR-449b, hsa-miR-450b-3p, hsa-miR-450b-5p, hsa-miR-484, hsa-miR-485-3p, hsa-miR-489, hsa-miR-490-5p, hsa-miR-495, hsa-miR-497, hsa-miR-498, hsa-miR-499-5p, hsa-miR-506, hsa-miR-507, hsa-miR-507, hsa-miR-508-5p, hsa-miR-509-3p, hsa-miR-509-3p, hsa-miR-512-5p, hsa-miR-513a-3p, hsa-miR-518a-5p, hsa-miR-518a-5p, hsa-miR-520a*, hsa-miR-520d-5p, hsa-miR-524-5p, hsa-miR-525, hsa-miR-527, hsa-miR-527, hsa-miR-548a-5p, hsa-miR-548b-5p, hsa-miR-548c-3p, hsa-miR-548c-5p, hsa-miR-548d-3p, hsa-miR-548d-5p, hsa-miR-548g, hsa-miR-548h, hsa-miR-548i, hsa-miR-548j, hsa-miR-548l, hsa-miR-548o, hsa-miR-551b*, hsa-miR-557, hsa-miR-558, hsa-miR-559, hsa-miR-561, hsa-miR-567, hsa-miR-569, hsa-miR-574-5p, hsa-miR-576, hsa-miR-576-5p, hsa-miR-578, hsa-miR-579, hsa-miR-579, hsa-miR-584, hsa-miR-586, hsa-miR-589*, hsa-miR-590-5p, hsa-miR-590-5p, hsa-miR-598, hsa-miR-606, hsa-miR-607, hsa-miR-607, hsa-miR-612, hsa-miR-624, hsa-miR-625, hsa-miR-626, hsa-miR-628-5p, hsa-miR-629*, hsa-miR-630, hsa-miR-634, hsa-miR-638, hsa-miR-642, hsa-miR-649, hsa-miR-653, hsa-miR-655, hsa-miR-656, hsa-miR-769-3p, hsa-miR-770-5p, hsa-miR-9*, hsa-miR-920, hsa-miR-922, hsa-miR-93*, hsa-miR-936, hsa-miR-940, and hsa-miR-944.

Sox2—SOX2—NM_{—}003106—hsa-abi-13242, hsa-abi-13248, hsa-abi-13259, hsa-abi-6775, hsa-let-7a*, hsa-let-7b*, hsa-let-7f-1*, hsa-miR-1246, hsa-miR-126, hsa-miR-1262, hsa-miR-1282, hsa-miR-1284, hsa-miR-1290, hsa-miR-129-5p, hsa-miR-1301, hsa-miR-140-3p, hsa-miR-145, hsa-miR-182, hsa-miR-200b, hsa-miR-200c, hsa-miR-21, hsa-miR-218-2*, hsa-miR-300, hsa-miR-30a*, hsa-miR-30d*, hsa-miR-30e*, hsa-miR-32*, hsa-miR-320, hsa-miR-369-3p, hsa-miR-371-5p, hsa-miR-381, hsa-miR-429, hsa-miR-432, hsa-miR-432*, hsa-miR-450b-3p, hsa-miR-450b-5p, hsa-miR-490-5p, hsa-miR-507, hsa-miR-509-3p, hsa-miR-513a-3p, hsa-miR-518a-5p, hsa-miR-520a*, hsa-miR-525, hsa-miR-527, hsa-miR-548g, hsa-miR-557, hsa-miR-561, hsa-miR-574-5p, hsa-miR-576-5p, hsa-miR-578, hsa-miR-579, hsa-miR-590-5p, hsa-miR-607, hsa-miR-624, hsa-miR-625, hsa-miR-626, hsa-miR-629*, hsa-miR-638, hsa-miR-655, hsa-miR-656, hsa-miR-769-3p, hsa-miR-770-5p, and hsa-miR-9*.

CD133—PROM1—NM_{—}006017—hsa-miR-124*, hsa-miR-1246, hsa-miR-1299, hsa-miR-1305, hsa-miR-130a*, hsa-miR-1321, hsa-miR-135a, hsa-miR-135b, hsa-miR-142-3p, hsa-miR-142-5p, hsa-miR-149*, hsa-miR-216b, hsa-miR-218, hsa-miR-219-2-3p, hsa-miR-22*, hsa-miR-23a, hsa-miR-23b, hsa-miR-29a, hsa-miR-29b, hsa-miR-29c, hsa-miR-32*, hsa-miR-337-3p, hsa-miR-33a*, hsa-miR-362-5p, hsa-miR-369-3p, hsa-miR-374a, hsa-miR-374b, hsa-miR-375, hsa-miR-377, hsa-miR-495, hsa-miR-501-5p, hsa-miR-513c, hsa-miR-542-3p, hsa-miR-548a-5p, hsa-miR-548b-5p, hsa-miR-548c-3p, hsa-miR-548c-5p, hsa-miR-548d-5p, hsa-miR-548g, hsa-miR-548h, hsa-miR-548i, hsa-miR-548j, hsa-miR-548l, hsa-miR-548m, hsa-miR-548n, hsa-miR-559, hsa-miR-579, hsa-miR-582-3p, hsa-miR-591, hsa-miR-603, hsa-miR-607, hsa-miR-625, hsa-miR-631, hsa-miR-637, hsa-miR-657, hsa-miR-758, hsa-miR-885-5p, and hsa-miR-888.

CD44—CD44—NM_{—}000610—hsa-miR-124*, hsa-miR-1246, hsa-miR-1299, hsa-miR-1305, hsa-miR-130a*, hsa-miR-1321, hsa-miR-135a, hsa-miR-135b, hsa-miR-142-3p, hsa-miR-142-5p, hsa-miR-149*, hsa-miR-216b, hsa-miR-218, hsa-miR-219-2-3p, hsa-miR-22*, hsa-miR-23a, hsa-miR-23b, hsa-miR-29a, hsa-miR-29b, hsa-miR-29c, hsa-miR-32*, hsa-miR-337-3p, hsa-miR-33a*, has-miR-34a, has-miR-34b, has-miR-34c, hsa-miR-362-5p, hsa-miR-369-3p, hsa-miR-374a, hsa-miR-374b, hsa-miR-377, hsa-miR-495, hsa-miR-501-5p, hsa-miR-513c, hsa-miR-542-3p, hsa-miR-548a-5p, hsa-miR-548b-5p, hsa-miR-548c-3p, hsa-miR-548c-5p, hsa-miR-548d-5p, hsa-miR-548g, hsa-miR-548h, hsa-miR-548i, hsa-miR-548j, hsa-miR-548l, hsa-miR-548m, hsa-miR-548n, hsa-miR-559, hsa-miR-579, hsa-miR-582-3p, hsa-miR-591, hsa-miR-603, hsa-miR-607, hsa-miR-625, hsa-miR-631, hsa-miR-637, hsa-miR-657, hsa-miR-758, hsa-miR-885-5p, and hsa-miR-888.

Bmi-1—BMI1/PCFG4—NM_{—}005180—hsa-abi-13272, hsa-abi-408, hsa-miR-1245, hsa-miR-1248, hsa-miR-1256, hsa-miR-1262, hsa-miR-1264, hsa-miR-128, hsa-miR-1283, hsa-miR-1290, hsa-miR-132, hsa-miR-142-5p, hsa-miR-154, hsa-miR-154*, hsa-miR-15b*, hsa-miR-16-2*, hsa-miR-183, hsa-miR-187*, hsa-miR-192*, hsa-miR-193b*, hsa-miR-194, hsa-miR-195*, hsa-miR-19a*, hsa-miR-19b-1*, hsa-miR-19b-2*, hsa-miR-200b, hsa-miR-200c, hsa-miR-203, hsa-miR-208a, hsa-miR-208b, hsa-miR-212, hsa-miR-218, hsa-miR-219-2-3p, hsa-miR-221, hsa-miR-222, hsa-miR-224, hsa-miR-27a, hsa-miR-27b, hsa-miR-298, hsa-miR-29b-1*, hsa-miR-302f, hsa-miR-30a, hsa-miR-30b, hsa-miR-30c, hsa-miR-30c-1*, hsa-miR-30c-2*, hsa-miR-30d, hsa-miR-30e, hsa-miR-32*, hsa-miR-320a, hsa-miR-320b, hsa-miR-320c, hsa-miR-320d, hsa-miR-329, hsa-miR-330-3p, hsa-miR-338-5p, hsa-miR-33a*, hsa-miR-340, hsa-miR-362-3p, hsa-miR-370, hsa-miR-376c, hsa-miR-380, hsa-miR-429, hsa-miR-431, hsa-miR-448, hsa-miR-452, hsa-miR-455-5p, hsa-miR-485-3p, hsa-miR-487a, hsa-miR-493-5p, hsa-miR-495, hsa-miR-501-5p, hsa-miR-508-3p, hsa-miR-509-3-5p, hsa-miR-509-5p, hsa-miR-513a-3p, hsa-miR-518a-5p, hsa-miR-520d, hsa-miR-524, hsa-miR-527, hsa-miR-532-5p, hsa-miR-545*, hsa-miR-548a-3p, hsa-miR-548a-5p, hsa-miR-548b-5p, hsa-miR-548c-5p, hsa-miR-548d-5p, hsa-miR-548e, hsa-miR-548f, hsa-miR-548h, hsa-miR-548i, hsa-miR-548j, hsa-miR-548l, hsa-miR-548n, hsa-miR-548p, hsa-miR-551b*, hsa-miR-553, hsa-miR-559, hsa-miR-576-3p, hsa-miR-576-5p, hsa-miR-577, hsa-miR-586, hsa-miR-587, hsa-miR-590-3p, hsa-miR-624, hsa-miR-629, hsa-miR-636, hsa-miR-758, hsa-miR-802, and hsa-miR-92a-2*.

ABCG2—ABCG2/BCRP—NM_{—}004827—hsa-miR-105, hsa-miR-106a, hsa-miR-106b, hsa-miR-10b*, hsa-miR-1185, hsa-miR-1200, hsa-miR-1228, hsa-miR-124*, hsa-miR-1252, hsa-miR-1284, hsa-miR-1322, hsa-miR-134, hsa-miR-135b*, hsa-miR-138-2*, hsa-miR-141*, hsa-miR-142-5p, hsa-miR-143*, hsa-miR-145*, hsa-miR-146a, hsa-miR-146b-5p, hsa-miR-148a*, hsa-miR-150*, hsa-miR-154*, hsa-miR-17, hsa-miR-181a-2*, hsa-miR-183*, hsa-miR-186, hsa-miR-187*, hsa-miR-199a-5p, hsa-miR-199b-5p, hsa-miR-203, hsa-miR-20a, hsa-miR-20b, hsa-miR-218, hsa-miR-302a, hsa-miR-302b, hsa-miR-302c, hsa-miR-302c*, hsa-miR-302d, hsa-miR-302e, hsa-miR-302f, hsa-miR-30c-1*, hsa-miR-30c-2*, hsa-miR-323-3p, hsa-miR-328, hsa-miR-372, hsa-miR-373, hsa-miR-378, hsa-miR-411, hsa-miR-422a, hsa-miR-450b-5p, hsa-miR-487a, hsa-miR-491-5p, hsa-miR-497*, hsa-miR-499-5p, hsa-miR-508-3p, hsa-miR-512-3p, hsa-miR-516b, hsa-miR-519d, hsa-miR-520a-3p, hsa-miR-520b, hsa-miR-520c-3p, hsa-miR-520d-3p, hsa-miR-520e, hsa-miR-520f, hsa-miR-520g, hsa-miR-520h, hsa-miR-522, hsa-miR-526b*, hsa-miR-542-3p, hsa-miR-548a-3p, hsa-miR-548e, hsa-miR-548f, hsa-miR-548l, hsa-miR-548n, hsa-miR-555, hsa-miR-561, hsa-miR-570, hsa-miR-577, hsa-miR-583, hsa-miR-590-3p, hsa-miR-619, hsa-miR-633, hsa-miR-651, hsa-miR-655, hsa-miR-656, hsa-miR-664, hsa-miR-665, hsa-miR-7-1*, hsa-miR-7-2*, hsa-miR-758, hsa-miR-873, hsa-miR-888*, hsa-miR-890, hsa-miR-891b, hsa-miR-93, and hsa-miR-944.

ABCB1—ABCB1—NM_{—}000927—hsa-let-7a*, hsa-let-7b*, hsa-let-7f-1*, hsa-miR-101*, hsa-miR-1253, hsa-miR-129-5p, hsa-miR-138-1*, hsa-miR-146a*, hsa-miR-16-2*, hsa-miR-188-3p, hsa-miR-195*, hsa-miR-214, hsa-miR-218-2*, hsa-miR-223, hsa-miR-298, hsa-miR-302c*, hsa-miR-331-5p, hsa-miR-338-5p, hsa-miR-34a*, hsa-miR-34b*, hsa-miR-455-3p, hsa-miR-485-3p, hsa-miR-491-3p, hsa-miR-508-5p, hsa-miR-513a-5p, hsa-miR-548l, hsa-miR-548n, hsa-miR-561, hsa-miR-593, hsa-miR-7-1*, hsa-miR-7-2*, hsa-miR-875-5p, hsa-miR-877*, hsa-miR-935, and hsa-miR-944.

ITGA2—ITGA2—NM_{—}002203—hsa-let-7a*, hsa-let-7b*, hsa-let-7f-1*, hsa-let-7f-2*, hsa-miR-101, hsa-miR-103, hsa-miR-106a, hsa-miR-106b, hsa-miR-107, hsa-miR-10a, hsa-miR-10b, hsa-miR-1182, hsa-miR-1200, hsa-miR-1224-5p, hsa-miR-1226, hsa-miR-1231, hsa-miR-1237, hsa-miR-1255a, hsa-miR-1255b, hsa-miR-1256, hsa-miR-1258, hsa-miR-125b-2*, hsa-miR-126*, hsa-miR-1263, hsa-miR-1265, hsa-miR-1267, hsa-miR-1269, hsa-miR-1272, hsa-miR-1283, hsa-miR-1288, hsa-miR-1290, hsa-miR-1293, hsa-miR-1297, hsa-miR-1303, hsa-miR-1321, hsa-miR-134, hsa-miR-135a, hsa-miR-135b, hsa-miR-138-2*, hsa-miR-139-5p, hsa-miR-140-5p, hsa-miR-141*, hsa-miR-142-3p, hsa-miR-143*, hsa-miR-146a, hsa-miR-146a*, hsa-miR-146b-3p, hsa-miR-146b-5p, hsa-miR-150, hsa-miR-155*, hsa-miR-15a, hsa-miR-15b, hsa-miR-16, hsa-miR-16-2*, hsa-miR-17, hsa-miR-17*, hsa-miR-181a, hsa-miR-181b, hsa-miR-181c, hsa-miR-181d, hsa-miR-1825, hsa-miR-1826, hsa-miR-185, hsa-miR-186, hsa-miR-186*, hsa-miR-18a, hsa-miR-18b, hsa-miR-190, hsa-miR-190b, hsa-miR-193a-5p, hsa-miR-193b*, hsa-miR-195, hsa-miR-195*, hsa-miR-198, hsa-miR-19a, hsa-miR-19b, hsa-miR-200a*, hsa-miR-200b, hsa-miR-200b*, hsa-miR-200c, hsa-miR-200c*, hsa-miR-202, hsa-miR-203, hsa-miR-20a, hsa-miR-20b, hsa-miR-20b*, hsa-miR-21, hsa-miR-21*, hsa-miR-216b, hsa-miR-219-5p, hsa-miR-223*, hsa-miR-24, hsa-miR-26a, hsa-miR-26b, hsa-miR-27a, hsa-miR-27b, hsa-miR-27b*, hsa-miR-297, hsa-miR-299-3p, hsa-miR-29a, hsa-miR-29b, hsa-miR-29c, hsa-miR-302a, hsa-miR-302b, hsa-miR-302c, hsa-miR-302c*, hsa-miR-302d, hsa-miR-302e, hsa-miR-30a, hsa-miR-30a*, hsa-miR-30b, hsa-miR-30c, hsa-miR-30c-1*, hsa-miR-30c-2*, hsa-miR-30d, hsa-miR-30d*, hsa-miR-30e, hsa-miR-30e*, hsa-miR-324-3p, hsa-miR-326, hsa-miR-329, hsa-miR-330-5p, hsa-miR-331-5p, hsa-miR-335*, hsa-miR-338-5p, hsa-miR-339-5p, hsa-miR-33a*, hsa-miR-33b*, hsa-miR-340, hsa-miR-342-5p, hsa-miR-34b, hsa-miR-361-3p, hsa-miR-362-3p, hsa-miR-371-5p, hsa-miR-372, hsa-miR-373, hsa-miR-374a, hsa-miR-374b, hsa-miR-376a, hsa-miR-376b, hsa-miR-378, hsa-miR-378*, hsa-miR-382, hsa-miR-422a, hsa-miR-424, hsa-miR-429, hsa-miR-431, hsa-miR-432, hsa-miR-452*, hsa-miR-486-3p, hsa-miR-489, hsa-miR-491-3p, hsa-miR-495, hsa-miR-497, hsa-miR-498, hsa-miR-500*, hsa-miR-504, hsa-miR-508-5p, hsa-miR-509-3-5p, hsa-miR-509-5p, hsa-miR-511, hsa-miR-513a-3p, hsa-miR-513c, hsa-miR-515-3p, hsa-miR-516b, hsa-miR-517*, hsa-miR-518d-5p, hsa-miR-518e*, hsa-miR-518f*, hsa-miR-519a*, hsa-miR-519b-5p, hsa-miR-519c-5p, hsa-miR-519d, hsa-miR-519e, hsa-miR-520a-3p, hsa-miR-520b, hsa-miR-520c-3p, hsa-miR-520c-5p, hsa-miR-520d-3p, hsa-miR-520d-5p, hsa-miR-520e, hsa-miR-520f, hsa-miR-520g, hsa-miR-520h, hsa-miR-522, hsa-miR-522*, hsa-miR-523*, hsa-miR-524-5p, hsa-miR-526a, hsa-miR-526b*, hsa-miR-532-5p, hsa-miR-539, hsa-miR-543, hsa-miR-544, hsa-miR-545, hsa-miR-548a-5p, hsa-miR-548b-5p, hsa-miR-548c-5p, hsa-miR-548d-3p, hsa-miR-548d-5p, hsa-miR-548h, hsa-miR-548i, hsa-miR-548j, hsa-miR-548n, hsa-miR-548p, hsa-miR-550, hsa-miR-550*, hsa-miR-551b*, hsa-miR-552, hsa-miR-559, hsa-miR-562, hsa-miR-567, hsa-miR-568, hsa-miR-575, hsa-miR-578, hsa-miR-581, hsa-miR-582-5p, hsa-miR-583, hsa-miR-586, hsa-miR-588, hsa-miR-590-3p, hsa-miR-590-5p, hsa-miR-595, hsa-miR-606, hsa-miR-607, hsa-miR-624, hsa-miR-634, hsa-miR-636, hsa-miR-640, hsa-miR-643, hsa-miR-645, hsa-miR-646, hsa-miR-649, hsa-miR-650, hsa-miR-653, hsa-miR-654-3p, hsa-miR-656, hsa-miR-664, hsa-miR-665, hsa-miR-671-3p, hsa-miR-7-1*, hsa-miR-7-2*, hsa-miR-765, hsa-miR-766, hsa-miR-768-3p, hsa-miR-768-5p, hsa-miR-873, hsa-miR-874, hsa-miR-875-3p, hsa-miR-889, hsa-miR-890, hsa-miR-891b, hsa-miR-924, hsa-miR-93, hsa-miR-93*, and hsa-miR-939.

ITGB1—TGB1—NM_{—}002211—hsa-miR-100*, hsa-miR-1226, hsa-miR-124, hsa-miR-1264, hsa-miR-1279, hsa-miR-1286, hsa-miR-129-5p, hsa-miR-1300, hsa-miR-134, hsa-miR-138-2*, hsa-miR-144, hsa-miR-146a*, hsa-miR-153, hsa-miR-16-2*, hsa-miR-183, hsa-miR-183*, hsa-miR-18b*, hsa-miR-190, hsa-miR-190b, hsa-miR-195*, hsa-miR-200c*, hsa-miR-221*, hsa-miR-223, hsa-miR-297, hsa-miR-29a, hsa-miR-29b, hsa-miR-29c, hsa-miR-302b*, hsa-miR-302d*, hsa-miR-302f, hsa-miR-30a, hsa-miR-30b, hsa-miR-30c, hsa-miR-30d, hsa-miR-30e, hsa-miR-367*, hsa-miR-373*, hsa-miR-374b*, hsa-miR-423-3p, hsa-miR-493*, hsa-miR-493-5p, hsa-miR-499-5p, hsa-miR-500, hsa-miR-506, hsa-miR-507, hsa-miR-511, hsa-miR-513a-3p, hsa-miR-513c, hsa-miR-522, hsa-miR-542-3p, hsa-miR-544, hsa-miR-545, hsa-miR-545*, hsa-miR-548a-5p, hsa-miR-548b-5p, hsa-miR-548c-3p, hsa-miR-548c-5p, hsa-miR-548d-5p, hsa-miR-548h, hsa-miR-548i, hsa-miR-548j, hsa-miR-548k, hsa-miR-548l, hsa-miR-548n, hsa-miR-548p, hsa-miR-550*, hsa-miR-551b*, hsa-miR-557, hsa-miR-559, hsa-miR-616*, hsa-miR-621, hsa-miR-634, hsa-miR-641, hsa-miR-655, hsa-miR-7-1*, hsa-miR-7-2*, hsa-miR-768-3p, hsa-miR-888, hsa-miR-888*, and hsa-miR-9*.

PTEN—PTEN—NM_{—}000314—hsa-abi-10411, hsa-abi-13106, hsa-abi-13159, hsa-abi-13252, hsa-abi-13253, hsa-abi-4400, hsa-abi-801, hsa-let-7i*, hsa-miR-100*, hsa-miR-103, hsa-miR-106a, hsa-miR-106b, hsa-miR-107, hsa-miR-10a, hsa-miR-10b, hsa-miR-10b*, hsa-miR-1179, hsa-miR-1183, hsa-miR-1227, hsa-miR-1238, hsa-miR-1244, hsa-miR-1255a, hsa-miR-1255b, hsa-miR-1259, hsa-miR-125b-2*, hsa-miR-1265, hsa-miR-1270, hsa-miR-1272, hsa-miR-1279, hsa-miR-1283, hsa-miR-1285, hsa-miR-1287, hsa-miR-1294, hsa-miR-1297, hsa-miR-1299, hsa-miR-1305, hsa-miR-130a, hsa-miR-130a*, hsa-miR-130b, hsa-miR-1323, hsa-miR-136, hsa-miR-139-5p, hsa-miR-140-3p, hsa-miR-141, hsa-miR-142-5p, hsa-miR-144, hsa-miR-148a, hsa-miR-148a*, hsa-miR-148b, hsa-miR-148b*, hsa-miR-150*, hsa-miR-152, hsa-miR-153, hsa-miR-16-1*, hsa-miR-16-2*, hsa-miR-17, hsa-miR-181a, hsa-miR-181b, hsa-miR-181c, hsa-miR-181d, hsa-miR-186, hsa-miR-187*, hsa-miR-188-5p, hsa-miR-193a-3p, hsa-miR-193b, hsa-miR-195*, hsa-miR-198, hsa-miR-19a, hsa-miR-19b, hsa-miR-200a, hsa-miR-200b, hsa-miR-200c, hsa-miR-205, hsa-miR-20a, hsa-miR-20b, hsa-miR-214, hsa-miR-216a, hsa-miR-219-2-3p, hsa-miR-22, hsa-miR-221, hsa-miR-23a, hsa-miR-23b, hsa-miR-25, hsa-miR-26a, hsa-miR-26a-1*, hsa-miR-26a-2*, hsa-miR-26b, hsa-miR-28-5p, hsa-miR-296-3p, hsa-miR-298, hsa-miR-29a, hsa-miR-29b, hsa-miR-29c, hsa-miR-301a, hsa-miR-301b, hsa-miR-30a*, hsa-miR-30d*, hsa-miR-30e*, hsa-miR-32, hsa-miR-320a, hsa-miR-320b, hsa-miR-320c, hsa-miR-320d, hsa-miR-325, hsa-miR-328, hsa-miR-335*, hsa-miR-337-3p, hsa-miR-337-5p, hsa-miR-338-3p, hsa-miR-338-5p, hsa-miR-33a*, hsa-miR-363, hsa-miR-363*, hsa-miR-367, hsa-miR-367*, hsa-miR-373*, hsa-miR-374a, hsa-miR-374b, hsa-miR-382, hsa-miR-424*, hsa-miR-425, hsa-miR-425-5p, hsa-miR-429, hsa-miR-454, hsa-miR-485-5p, hsa-miR-486-5p, hsa-miR-494, hsa-miR-495, hsa-miR-498, hsa-miR-505, hsa-miR-509-3p, hsa-miR-511, hsa-miR-513a-3p, hsa-miR-514, hsa-miR-518a-5p, hsa-miR-519a, hsa-miR-519b-3p, hsa-miR-519c-3p, hsa-miR-519d, hsa-miR-519e, hsa-miR-520a*, hsa-miR-520a-5p, hsa-miR-520b, hsa-miR-520c, hsa-miR-520d, hsa-miR-520d-5p, hsa-miR-520e, hsa-miR-524-5p, hsa-miR-525-5p, hsa-miR-526b*, hsa-miR-527, hsa-miR-532-5p, hsa-miR-542-3p, hsa-miR-543, hsa-miR-545, hsa-miR-548a-3p, hsa-miR-548a-5p, hsa-miR-548b-5p, hsa-miR-548c-3p, hsa-miR-548c-5p, hsa-miR-548d-3p, hsa-miR-548d-5p, hsa-miR-548e, hsa-miR-548f, hsa-miR-548h, hsa-miR-548i, hsa-miR-548j, hsa-miR-548l, hsa-miR-548m, hsa-miR-548n, hsa-miR-548o, hsa-miR-548p, hsa-miR-551b*, hsa-miR-554, hsa-miR-559, hsa-miR-561, hsa-miR-568, hsa-miR-569, hsa-miR-570, hsa-miR-577, hsa-miR-578, hsa-miR-579, hsa-miR-582-3p, hsa-miR-582-5p, hsa-miR-584, hsa-miR-586, hsa-miR-589*, hsa-miR-590-3p, hsa-miR-607, hsa-miR-612, hsa-miR-616, hsa-miR-616*, hsa-miR-618, hsa-miR-620, hsa-miR-622, hsa-miR-624, hsa-miR-628-3p, hsa-miR-631, hsa-miR-642, hsa-miR-649, hsa-miR-653, hsa-miR-664, hsa-miR-708, hsa-miR-7-1*, hsa-miR-7-2*, hsa-miR-744*, hsa-miR-768-3p, hsa-miR-875-3p, hsa-miR-888*, hsa-miR-889, hsa-miR-9*, hsa-miR-92a, hsa-miR-92b, hsa-miR-93, hsa-miR-939, and hsa-miR-944.

Stat3—STAT3—NM_{—}139276—hsa-abi-13160, hsa-abi-13252, hsa-abi-9134, hsa-let-7c*, hsa-miR-106a, hsa-miR-106a*, hsa-miR-106b, hsa-miR-10a, hsa-miR-10b, hsa-miR-1184, hsa-miR-1200, hsa-miR-1207-5p, hsa-miR-122*, hsa-miR-1224-5p, hsa-miR-1226, hsa-miR-1227, hsa-miR-1229, hsa-miR-124, hsa-miR-1243, hsa-miR-1244, hsa-miR-1251, hsa-miR-1252, hsa-miR-1255a, hsa-miR-1255b, hsa-miR-125a-5p, hsa-miR-125b, hsa-miR-1274b, hsa-miR-129-5p, hsa-miR-1299, hsa-miR-1301, hsa-miR-1303, hsa-miR-1305, hsa-miR-1308, hsa-miR-130a, hsa-miR-130b, hsa-miR-1321, hsa-miR-138-2*, hsa-miR-143*, hsa-miR-144*, hsa-miR-145*, hsa-miR-146a*, hsa-miR-149*, hsa-miR-150, hsa-miR-15a*, hsa-miR-17, hsa-miR-17-5p, hsa-miR-181a, hsa-miR-181b, hsa-miR-181c, hsa-miR-181d, hsa-miR-1827, hsa-miR-186*, hsa-miR-187*, hsa-miR-193b*, hsa-miR-202, hsa-miR-20a, hsa-miR-20b, hsa-miR-21, hsa-miR-21*, hsa-miR-214, hsa-miR-220b, hsa-miR-24, hsa-miR-26b*, hsa-miR-27a*, hsa-miR-296-5p, hsa-miR-298, hsa-miR-29a, hsa-miR-29b, hsa-miR-29c, hsa-miR-301a, hsa-miR-301b, hsa-miR-302a, hsa-miR-302a*, hsa-miR-302b, hsa-miR-302c, hsa-miR-302d, hsa-miR-302e, hsa-miR-31*, hsa-miR-320a, hsa-miR-320b, hsa-miR-320c, hsa-miR-320d, hsa-miR-323-3p, hsa-miR-337-3p, hsa-miR-338-5p, hsa-miR-34a, hsa-miR-34c-5p, hsa-miR-372, hsa-miR-373, hsa-miR-374a*, hsa-miR-376a*, hsa-miR-378*, hsa-miR-410, hsa-miR-421, hsa-miR-425, hsa-miR-431*, hsa-miR-449a, hsa-miR-449b, hsa-miR-454, hsa-miR-483-5p, hsa-miR-485-5p, hsa-miR-486-3p, hsa-miR-488, hsa-miR-488*, hsa-miR-493, hsa-miR-495, hsa-miR-497*, hsa-miR-498, hsa-miR-504, hsa-miR-506, hsa-miR-512-3p, hsa-miR-516-5p, hsa-miR-519a, hsa-miR-519b-3p, hsa-miR-519c-3p, hsa-miR-519d, hsa-miR-519e, hsa-miR-520a-3p, hsa-miR-520a-5p, hsa-miR-520b, hsa-miR-520c-3p, hsa-miR-520d-3p, hsa-miR-520d-5p, hsa-miR-520e, hsa-miR-520g, hsa-miR-520h, hsa-miR-524-5p, hsa-miR-525-5p, hsa-miR-526b*, hsa-miR-544, hsa-miR-548b-3p, hsa-miR-548c-3p, hsa-miR-548d-3p, hsa-miR-566, hsa-miR-577, hsa-miR-590-5p, hsa-miR-593*, hsa-miR-600, hsa-miR-608, hsa-miR-629*, hsa-miR-641, hsa-miR-642, hsa-miR-646, hsa-miR-648, hsa-miR-649, hsa-miR-650, hsa-miR-663, hsa-miR-664*, hsa-miR-665, hsa-miR-671-5p, hsa-miR-765, hsa-miR-874, hsa-miR-876-3p, hsa-miR-877*, hsa-miR-885-5p, hsa-miR-886-5p, hsa-miR-892b, hsa-miR-922, hsa-miR-92a-2*, hsa-miR-93, hsa-miR-93*, hsa-miR-940, and hsa-miR-96*.

Sox4—SOX4—NM_{—}003107—hsa-abi-12897, hsa-abi-13252, hsa-abi-3135, hsa-abi-685, hsa-abi-7510, hsa-abi-9651, hsa-let-7c*, hsa-let-7f-2*, hsa-miR-106a, hsa-miR-106b, hsa-miR-10a*, hsa-miR-1203, hsa-miR-1229, hsa-miR-1236, hsa-miR-1237, hsa-miR-1248, hsa-miR-1251, hsa-miR-1252, hsa-miR-1253, hsa-miR-1274b, hsa-miR-1275, hsa-miR-1277, hsa-miR-1279, hsa-miR-1281, hsa-miR-1285, hsa-miR-129*, hsa-miR-129-3p, hsa-miR-129-5p, hsa-miR-130a, hsa-miR-130b, hsa-miR-132, hsa-miR-1321, hsa-miR-133a, hsa-miR-133b, hsa-miR-138, hsa-miR-140-5p, hsa-miR-145, hsa-miR-146b-3p, hsa-miR-149*, hsa-miR-155*, hsa-miR-16-1*, hsa-miR-17, hsa-miR-181a-2*, hsa-miR-185*, hsa-miR-188-5p, hsa-miR-191, hsa-miR-191*, hsa-miR-19a, hsa-miR-19b, hsa-miR-204, hsa-miR-20a, hsa-miR-20b, hsa-miR-21*, hsa-miR-211, hsa-miR-212, hsa-miR-214*, hsa-miR-219-2-3p, hsa-miR-221, hsa-miR-222, hsa-miR-25, hsa-miR-25*, hsa-miR-299-5p, hsa-miR-29a*, hsa-miR-300, hsa-miR-301a, hsa-miR-301b, hsa-miR-30a, hsa-miR-30a*, hsa-miR-30b, hsa-miR-30c, hsa-miR-30d, hsa-miR-30d*, hsa-miR-30e, hsa-miR-30e*, hsa-miR-32, hsa-miR-320a, hsa-miR-320b, hsa-miR-320c, hsa-miR-320d, hsa-miR-324-3p, hsa-miR-335*, hsa-miR-337, hsa-miR-338, hsa-miR-339-5p, hsa-miR-33b*, hsa-miR-342-5p, hsa-miR-34a, hsa-miR-34b, hsa-miR-34b*, hsa-miR-34c-5p, hsa-miR-363, hsa-miR-363*, hsa-miR-367, hsa-miR-369-3p, hsa-miR-373*, hsa-miR-374a, hsa-miR-374b, hsa-miR-381, hsa-miR-384, hsa-miR-421, hsa-miR-432*, hsa-miR-449a, hsa-miR-449b, hsa-miR-450, hsa-miR-454, hsa-miR-483-5p, hsa-miR-486-5p, hsa-miR-497*, hsa-miR-498, hsa-miR-499-5p, hsa-miR-500, hsa-miR-501-5p, hsa-miR-511, hsa-miR-513a-5p, hsa-miR-515-3p, hsa-miR-517*, hsa-miR-518c*, hsa-miR-519a, hsa-miR-519b-3p, hsa-miR-519c-3p, hsa-miR-519d, hsa-miR-519e, hsa-miR-520b, hsa-miR-520c, hsa-miR-520d-5p, hsa-miR-524*, hsa-miR-524-5p, hsa-miR-526b*, hsa-miR-539, hsa-miR-541*, hsa-miR-548a-3p, hsa-miR-548c-3p, hsa-miR-548d-3p, hsa-miR-548e, hsa-miR-548f, hsa-miR-548g, hsa-miR-548p, hsa-miR-550, hsa-miR-561, hsa-miR-571, hsa-miR-573, hsa-miR-576-3p, hsa-miR-583, hsa-miR-590-3p, hsa-miR-595, hsa-miR-596, hsa-miR-600, hsa-miR-603, hsa-miR-612, hsa-miR-616*, hsa-miR-618, hsa-miR-622, hsa-miR-623, hsa-miR-629*, hsa-miR-637, hsa-miR-649, hsa-miR-655, hsa-miR-656, hsa-miR-657, hsa-miR-658, hsa-miR-767-3p, hsa-miR-877*, hsa-miR-888, hsa-miR-892a, hsa-miR-892b, hsa-miR-9*, hsa-miR-92a, hsa-miR-92a-2*, hsa-miR-92b, hsa-miR-93, hsa-miR-939, hsa-miR-99a*, and hsa-miR-99b*.

ALDH1—ALDH1A1—NM_{—}000689—hsa-miR-126*, hsa-miR-1283, hsa-miR-130a*, hsa-miR-132, hsa-miR-134, hsa-miR-154*, hsa-miR-17*, hsa-miR-187*, hsa-miR-21, hsa-miR-212, hsa-miR-221, hsa-miR-222, hsa-miR-23a, hsa-miR-23b, hsa-miR-297, hsa-miR-335*, hsa-miR-455-3p, hsa-miR-485-3p, hsa-miR-487a, hsa-miR-489, hsa-miR-511, hsa-miR-520d-5p, hsa-miR-524-5p, hsa-miR-548c-3p, hsa-miR-548n, hsa-miR-551b*, hsa-miR-562, hsa-miR-567, hsa-miR-590-5p, hsa-miR-606, hsa-miR-643, hsa-miR-651, hsa-miR-942, hsa-miR-943, and hsa-miR-944.

ALDH1A2—Isoform 1—NM_{—}003888—hsa-abi-13135, hsa-abi-9651, hsa-let-7c*, hsa-let-7g*, hsa-miR-103, hsa-miR-107, hsa-miR-10b*, hsa-miR-1179, hsa-miR-1184, hsa-miR-1200, hsa-miR-1207-3p, hsa-miR-1228, hsa-miR-1243, hsa-miR-1248, hsa-miR-1251, hsa-miR-1252, hsa-miR-1254, hsa-miR-1261, hsa-miR-1262, hsa-miR-1272, hsa-miR-1300, hsa-miR-1303, hsa-miR-130a*, hsa-miR-132*, hsa-miR-1321, hsa-miR-1323, hsa-miR-137, hsa-miR-142-3p, hsa-miR-143*, hsa-miR-148a*, hsa-miR-149*, hsa-miR-151-5p, hsa-miR-15a*, hsa-miR-16-2*, hsa-miR-182, hsa-miR-182*, hsa-miR-188-3p, hsa-miR-192*, hsa-miR-195*, hsa-miR-19a*, hsa-miR-19b-1*, hsa-miR-19b-2*, hsa-miR-203, hsa-miR-204, hsa-miR-20a*, hsa-miR-20b*, hsa-miR-211, hsa-miR-221*, hsa-miR-23a, hsa-miR-23b, hsa-miR-24-1*, hsa-miR-24-2*, hsa-miR-28-5p, hsa-miR-298, hsa-miR-29b-1*, hsa-miR-31, hsa-miR-324-3p, hsa-miR-326, hsa-miR-329, hsa-miR-330-5p, hsa-miR-335*, hsa-miR-337, hsa-miR-338-3p, hsa-miR-338-5p, hsa-miR-345, hsa-miR-362-3p, hsa-miR-369-3p, hsa-miR-423-5p, hsa-miR-432*, hsa-miR-453, hsa-miR-455-3p, hsa-miR-483-3p, hsa-miR-489, hsa-miR-490-5p, hsa-miR-502-5p, hsa-miR-509-3-5p, hsa-miR-509-5p, hsa-miR-542-3p, hsa-miR-548a-3p, hsa-miR-548e, hsa-miR-548f, hsa-miR-548g, hsa-miR-548m, hsa-miR-548o, hsa-miR-556-5p, hsa-miR-582-3p, hsa-miR-600, hsa-miR-608, hsa-miR-617, hsa-miR-621, hsa-miR-632, hsa-miR-635, hsa-miR-641, hsa-miR-645, hsa-miR-654-3p, hsa-miR-655, hsa-miR-659, hsa-miR-663b, hsa-miR-664, hsa-miR-708, hsa-miR-744*, hsa-miR-758, hsa-miR-768-3p, hsa-miR-875-3p, hsa-miR-890, and hsa-miR-93*.

ALDH1A2 isoform 2—NM_{—}170696—hsa-abi-13135, hsa-abi-9651, hsa-let-7c*, hsa-let-7g*, hsa-miR-103, hsa-miR-107, hsa-miR-10b*, hsa-miR-1179, hsa-miR-1184, hsa-miR-1200, hsa-miR-1207-3p, hsa-miR-1228, hsa-miR-1243, hsa-miR-1248, hsa-miR-1251, hsa-miR-1252, hsa-miR-1254, hsa-miR-1261, hsa-miR-1262, hsa-miR-1272, hsa-miR-1300, hsa-miR-1303, hsa-miR-130a*, hsa-miR-132*, hsa-miR-1321, hsa-miR-1323, hsa-miR-137, hsa-miR-142-3p, hsa-miR-143*, hsa-miR-148a*, hsa-miR-149*, hsa-miR-151-5p, hsa-miR-15a*, hsa-miR-16-2*, hsa-miR-182, hsa-miR-182*, hsa-miR-188-3p, hsa-miR-192*, hsa-miR-195*, hsa-miR-19a*, hsa-miR-19b-1*, hsa-miR-19b-2*, hsa-miR-203, hsa-miR-204, hsa-miR-20a*, hsa-miR-20b*, hsa-miR-211, hsa-miR-221*, hsa-miR-23a, hsa-miR-23b, hsa-miR-24-1*, hsa-miR-24-2*, hsa-miR-28-5p, hsa-miR-298, hsa-miR-29b-1*, hsa-miR-31, hsa-miR-324-3p, hsa-miR-326, hsa-miR-329, hsa-miR-330-5p, hsa-miR-335*, hsa-miR-337, hsa-miR-338-3p, hsa-miR-338-5p, hsa-miR-345, hsa-miR-362-3p, hsa-miR-369-3p, hsa-miR-423-5p, hsa-miR-432*, hsa-miR-453, hsa-miR-455-3p, hsa-miR-483-3p, hsa-miR-489, hsa-miR-490-5p, hsa-miR-502-5p, hsa-miR-509-3-5p, hsa-miR-509-5p, hsa-miR-542-3p, hsa-miR-548a-3p, hsa-miR-548e, hsa-miR-548f, hsa-miR-548g, hsa-miR-548m, hsa-miR-548o, hsa-miR-556-5p, hsa-miR-582-3p, hsa-miR-600, hsa-miR-608, hsa-miR-617, hsa-miR-621, hsa-miR-632, hsa-miR-635, hsa-miR-641, hsa-miR-645, hsa-miR-654-3p, hsa-miR-655, hsa-miR-659, hsa-miR-663b, hsa-miR-664, hsa-miR-708, hsa-miR-744*, hsa-miR-758, hsa-miR-768-3p, hsa-miR-875-3p, hsa-miR-890, and hsa-miR-93*.

ALDH1A2 isoform 3—NM_{—}170697—hsa-abi-13135, hsa-abi-9651, hsa-let-7c*, hsa-let-7g*, hsa-miR-103, hsa-miR-107, hsa-miR-10b*, hsa-miR-1179, hsa-miR-1184, hsa-miR-1200, hsa-miR-1207-3p, hsa-miR-1228, hsa-miR-1243, hsa-miR-1248, hsa-miR-1251, hsa-miR-1252, hsa-miR-1254, hsa-miR-1261, hsa-miR-1262, hsa-miR-1272, hsa-miR-1300, hsa-miR-1303, hsa-miR-130a*, hsa-miR-132*, hsa-miR-1321, hsa-miR-1323, hsa-miR-137, hsa-miR-142-3p, hsa-miR-143*, hsa-miR-148a*, hsa-miR-149*, hsa-miR-151-5p, hsa-miR-15a*, hsa-miR-16-2*, hsa-miR-182, hsa-miR-182*, hsa-miR-188-3p, hsa-miR-192*, hsa-miR-195*, hsa-miR-19a*, hsa-miR-19b-1*, hsa-miR-19b-2*, hsa-miR-203, hsa-miR-204, hsa-miR-20a*, hsa-miR-20b*, hsa-miR-211, hsa-miR-221*, hsa-miR-23a, hsa-miR-23b, hsa-miR-24-1*, hsa-miR-24-2*, hsa-miR-28-5p, hsa-miR-298, hsa-miR-29b-1*, hsa-miR-31, hsa-miR-324-3p, hsa-miR-326, hsa-miR-329, hsa-miR-330-5p, hsa-miR-335*, hsa-miR-337, hsa-miR-338-3p, hsa-miR-338-5p, hsa-miR-345, hsa-miR-362-3p, hsa-miR-369-3p, hsa-miR-423-5p, hsa-miR-432*, hsa-miR-453, hsa-miR-455-3p, hsa-miR-483-3p, hsa-miR-489, hsa-miR-490-5p, hsa-miR-502-5p, hsa-miR-509-3-5p, hsa-miR-509-5p, hsa-miR-542-3p, hsa-miR-548a-3p, hsa-miR-548e, hsa-miR-548f, hsa-miR-548g, hsa-miR-548m, hsa-miR-548o, hsa-miR-556-5p, hsa-miR-582-3p, hsa-miR-600, hsa-miR-608, hsa-miR-617, hsa-miR-621, hsa-miR-632, hsa-miR-635, hsa-miR-641, hsa-miR-645, hsa-miR-654-3p, hsa-miR-655, hsa-miR-659, hsa-miR-663b, hsa-miR-664, hsa-miR-708, hsa-miR-744*, hsa-miR-758, hsa-miR-768-3p, hsa-miR-875-3p, hsa-miR-890, and hsa-miR-93*.

ALDHIA3—NM_{—}000693—hsa-abi-13268, hsa-miR-101, hsa-miR-106a, hsa-miR-106b, hsa-miR-10b*, hsa-miR-1184, hsa-miR-1224-3p, hsa-miR-1225-3p, hsa-miR-1233, hsa-miR-1249, hsa-miR-125a-5p, hsa-miR-125b, hsa-miR-1260, hsa-miR-1274a, hsa-miR-1274b, hsa-miR-127-5p, hsa-miR-1286, hsa-miR-1288, hsa-miR-1301, hsa-miR-1323, hsa-miR-138, hsa-miR-140-3p, hsa-miR-144, hsa-miR-145, hsa-miR-15a, hsa-miR-15b, hsa-miR-16, hsa-miR-16-1*, hsa-miR-16-2*, hsa-miR-17, hsa-miR-183*, hsa-miR-186*, hsa-miR-188-3p, hsa-miR-193b*, hsa-miR-194, hsa-miR-195, hsa-miR-195*, hsa-miR-199a, hsa-miR-200b, hsa-miR-200c, hsa-miR-20a, hsa-miR-20b, hsa-miR-216a, hsa-miR-220c, hsa-miR-25, hsa-miR-29a*, hsa-miR-29b-1*, hsa-miR-30a*, hsa-miR-30d*, hsa-miR-30e*, hsa-miR-31*, hsa-miR-32, hsa-miR-324-5p, hsa-miR-326, hsa-miR-330-3p, hsa-miR-330-5p, hsa-miR-335, hsa-miR-338-5p, hsa-miR-33b*, hsa-miR-34b*, hsa-miR-34c-3p, hsa-miR-363, hsa-miR-367, hsa-miR-374b*, hsa-miR-379, hsa-miR-412, hsa-miR-424, hsa-miR-429, hsa-miR-450b-5p, hsa-miR-452*, hsa-miR-487b, hsa-miR-493, hsa-miR-497, hsa-miR-497*, hsa-miR-498, hsa-miR-500, hsa-miR-501-5p, hsa-miR-504, hsa-miR-505, hsa-miR-508-5p, hsa-miR-510, hsa-miR-515-3p, hsa-miR-519d, hsa-miR-519e, hsa-miR-526b*, hsa-miR-545, hsa-miR-548d-3p, hsa-miR-548k, hsa-miR-548l, hsa-miR-548m, hsa-miR-548n, hsa-miR-548o, hsa-miR-570, hsa-miR-577, hsa-miR-579, hsa-miR-583, hsa-miR-589, hsa-miR-589*, hsa-miR-590-3p, hsa-miR-603, hsa-miR-607, hsa-miR-618, hsa-miR-622, hsa-miR-641, hsa-miR-7, hsa-miR-766, hsa-miR-889, hsa-miR-890, hsa-miR-92a, hsa-miR-92b, hsa-miR-93, hsa-miR-936, and hsa-miR-940.

ALDH1B1—NM_{—}000692—hsa-let-7c*, hsa-miR-100*, hsa-miR-1178, hsa-miR-1179, hsa-miR-1224-3p, hsa-miR-1246, hsa-miR-1260, hsa-miR-1265, hsa-miR-1269, hsa-miR-1273, hsa-miR-1283, hsa-miR-129-5p, hsa-miR-1300, hsa-miR-134, hsa-miR-150, hsa-miR-154*, hsa-miR-15b*, hsa-miR-182, hsa-miR-185*, hsa-miR-187*, hsa-miR-188-3p, hsa-miR-193b*, hsa-miR-214, hsa-miR-219-1-3p, hsa-miR-221*, hsa-miR-222*, hsa-miR-24-1*, hsa-miR-24-2*, hsa-miR-25*, hsa-miR-27a*, hsa-miR-29a*, hsa-miR-29b-1*, hsa-miR-302f, hsa-miR-30b*, hsa-miR-331-3p, hsa-miR-363*, hsa-miR-377*, hsa-miR-378, hsa-miR-422a, hsa-miR-485-3p, hsa-miR-485-5p, hsa-miR-487a, hsa-miR-490-5p, hsa-miR-493*, hsa-miR-494, hsa-miR-508-5p, hsa-miR-510, hsa-miR-520a-5p, hsa-miR-522, hsa-miR-525-5p, hsa-miR-532-3p, hsa-miR-548p, hsa-miR-552, hsa-miR-581, hsa-miR-587, hsa-miR-589*, hsa-miR-592, hsa-miR-605, hsa-miR-609, hsa-miR-622, hsa-miR-658, hsa-miR-661, hsa-miR-7-1*, hsa-miR-7-2*, hsa-miR-766, hsa-miR-876-3p, hsa-miR-885-5p, hsa-miR-886-3p, hsa-miR-92a-1*, and hsa-miR-96*.

ALDH1L1—NM_{—}012190—hsa-miR-1207-5p, hsa-miR-1228, hsa-miR-1321, hsa-miR-149*, hsa-miR-296-5p, hsa-miR-30b*, hsa-miR-361-3p, hsa-miR-423-5p, hsa-miR-484, hsa-miR-486-3p, hsa-miR-629*, and hsa-miR-940.

Prostate stem cell antigen—PSCA—NM_{—}005672—hsa-abi-13100, hsa-miR-103, hsa-miR-107, hsa-miR-1184, hsa-miR-1200, hsa-miR-1205, hsa-miR-1207-5p, hsa-miR-1225-3p, hsa-miR-1258, hsa-miR-1265, hsa-miR-1275, hsa-miR-1289, hsa-miR-145, hsa-miR-149, hsa-miR-149*, hsa-miR-15a, hsa-miR-15b, hsa-miR-16, hsa-miR-195, hsa-miR-199a-5p, hsa-miR-199b-5p, hsa-miR-214*, hsa-miR-220c, hsa-miR-296-3p, hsa-miR-296-5p, hsa-miR-30b*, hsa-miR-33b*, hsa-miR-361-3p, hsa-miR-363*, hsa-miR-370, hsa-miR-376a, hsa-miR-376b, hsa-miR-423-5p, hsa-miR-424, hsa-miR-455-3p, hsa-miR-455-5p, hsa-miR-484, hsa-miR-485-5p, hsa-miR-497, hsa-miR-515-3p, hsa-miR-516a-3p, hsa-miR-516b, hsa-miR-516b*, hsa-miR-518c*, hsa-miR-519e, hsa-miR-532-3p, hsa-miR-541, hsa-miR-550, hsa-miR-552, hsa-miR-592, hsa-miR-593*, hsa-miR-608, hsa-miR-625, hsa-miR-631, hsa-miR-637, hsa-miR-651, hsa-miR-654-5p, hsa-miR-661, hsa-miR-675, hsa-miR-744, hsa-miR-768-5p, hsa-miR-873, hsa-miR-922, and hsa-miR-92a-2*.

Integrin, alpha V—ITGAV—NM_{—}002210—ambi_miR_{—}7079, hsa-let-7a*, hsa-let-7b*, hsa-let-7f-1*, hsa-let-7g*, hsa-miR-10b*, hsa-miR-1183, hsa-miR-1206, hsa-miR-1224-5p, hsa-miR-1229, hsa-miR-1244, hsa-miR-1246, hsa-miR-1252, hsa-miR-125a-3p, hsa-miR-125b-2*, hsa-miR-1260, hsa-miR-1262, hsa-miR-1263, hsa-miR-1264, hsa-miR-1269, hsa-miR-1276, hsa-miR-1284, hsa-miR-1288, hsa-miR-1290, hsa-miR-1303, hsa-miR-130b*, hsa-miR-1323, hsa-miR-135a, hsa-miR-135b, hsa-miR-136, hsa-miR-137, hsa-miR-141, hsa-miR-142-3p, hsa-miR-142-5p, hsa-miR-144, hsa-miR-144*, hsa-miR-150, hsa-miR-155*, hsa-miR-16-1*, hsa-miR-16-2*, hsa-miR-183, hsa-miR-186, hsa-miR-188-5p, hsa-miR-192, hsa-miR-192*, hsa-miR-194, hsa-miR-195*, hsa-miR-196a*, hsa-miR-197, hsa-miR-200a, hsa-miR-20b*, hsa-miR-215, hsa-miR-216b, hsa-miR-217, hsa-miR-224, hsa-miR-25, hsa-miR-297, hsa-miR-299-3p, hsa-miR-29a*, hsa-miR-300, hsa-miR-302a*, hsa-miR-302c*, hsa-miR-30a*, hsa-miR-30d*, hsa-miR-30e*, hsa-miR-31, hsa-miR-32, hsa-miR-323-3p, hsa-miR-328, hsa-miR-331-5p, hsa-miR-335*, hsa-miR-33a*, hsa-miR-340, hsa-miR-34b, hsa-miR-361-5p, hsa-miR-363, hsa-miR-367, hsa-miR-369-3p, hsa-miR-373*, hsa-miR-374a, hsa-miR-374b, hsa-miR-377, hsa-miR-380, hsa-miR-380*, hsa-miR-381, hsa-miR-432*, hsa-miR-433, hsa-miR-448, hsa-miR-450b-5p, hsa-miR-452*, hsa-miR-495, hsa-miR-496, hsa-miR-498, hsa-miR-509-3-5p, hsa-miR-509-3p, hsa-miR-509-5p, hsa-miR-513a-3p, hsa-miR-513b, hsa-miR-513c, hsa-miR-515-5p, hsa-miR-517*, hsa-miR-518d-5p, hsa-miR-518e*, hsa-miR-518f*, hsa-miR-519a*, hsa-miR-519b-5p, hsa-miR-519c-5p, hsa-miR-519e*, hsa-miR-520c-5p, hsa-miR-520g, hsa-miR-520h, hsa-miR-522*, hsa-miR-523*, hsa-miR-526a, hsa-miR-541*, hsa-miR-545, hsa-miR-545*, hsa-miR-548a-3p, hsa-miR-548a-5p, hsa-miR-548b-5p, hsa-miR-548c-3p, hsa-miR-548c-5p, hsa-miR-548d-5p, hsa-miR-548e, hsa-miR-548f, hsa-miR-548g, hsa-miR-548h, hsa-miR-548i, hsa-miR-548j, hsa-miR-548k, hsa-miR-548m, hsa-miR-548n, hsa-miR-548o, hsa-miR-548p, hsa-miR-549, hsa-miR-551b*, hsa-miR-555, hsa-miR-559, hsa-miR-561, hsa-miR-563, hsa-miR-569, hsa-miR-575, hsa-miR-576-5p, hsa-miR-577, hsa-miR-580, hsa-miR-582-3p, hsa-miR-589*, hsa-miR-590-3p, hsa-miR-605, hsa-miR-606, hsa-miR-607, hsa-miR-615-5p, hsa-miR-616*, hsa-miR-623, hsa-miR-624, hsa-miR-630, hsa-miR-641, hsa-miR-643, hsa-miR-647, hsa-miR-651, hsa-miR-654-3p, hsa-miR-655, hsa-miR-660, hsa-miR-664, hsa-miR-7-1*, hsa-miR-7-2*, hsa-miR-802, hsa-miR-875-5p, hsa-miR-885-5p, hsa-miR-888, hsa-miR-889, hsa-miR-891b, hsa-miR-9*, hsa-miR-92a, hsa-miR-92b, hsa-miR-943, hsa-miR-944, and hsa-miR-96*.

Nuclear factor kappa B1—NFKB1—NM_{—}003998—hsa-abi-9451, hsa-miR-1207-3p, hsa-miR-1252, hsa-miR-129-5p, hsa-miR-1322, hsa-miR-15a, hsa-miR-15b, hsa-miR-16, hsa-miR-183, hsa-miR-195, hsa-miR-199a-5p, hsa-miR-199b-5p, hsa-miR-214, hsa-miR-299-5p, hsa-miR-424, hsa-miR-450b-5p, hsa-miR-497, hsa-miR-508-3p, hsa-miR-516a-5p, hsa-miR-532-5p, hsa-miR-539, hsa-miR-548a-5p, hsa-miR-548b-5p, hsa-miR-548c-5p, hsa-miR-548d-5p, hsa-miR-548h, hsa-miR-548i, hsa-miR-548j, hsa-miR-548l, hsa-miR-548n, hsa-miR-548p, hsa-miR-559, hsa-miR-572, hsa-miR-575, R-579, hsa-miR-590-3p, hsa-miR-625, hsa-miR-640, hsa-miR-646, hsa-miR-664, hsa-miR-671-5p, and hsa-miR-9.

Bcl-2—BCL2 variant alpha—NM_{—}000633—hsa-abi-13135, hsa-abi-13156, hsa-abi-13159, hsa-abi-13248, hsa-abi-13268, hsa-let-7c*, hsa-let-7g*, hsa-miR-1, hsa-miR-100*, hsa-miR-101, hsa-miR-103, hsa-miR-106a, hsa-miR-106b, hsa-miR-107, hsa-miR-10a, hsa-miR-10b, hsa-miR-1178, hsa-miR-1184, hsa-miR-1201, hsa-miR-1204, hsa-miR-1225-5p, hsa-miR-1226*, hsa-miR-1228, hsa-miR-1229, hsa-miR-1231, hsa-miR-1233, hsa-miR-1234, hsa-miR-1237, hsa-miR-1243, hsa-miR-1246, hsa-miR-1250, hsa-miR-1251, hsa-miR-1257, hsa-miR-1259, hsa-miR-125a-5p, hsa-miR-125b, hsa-miR-126*, hsa-miR-1260, hsa-miR-1265, hsa-miR-1266, hsa-miR-1267, hsa-miR-1274b, hsa-miR-1276, hsa-miR-1285, hsa-miR-129*, hsa-miR-1290, hsa-miR-129-3p, hsa-miR-1299, hsa-miR-1301, hsa-miR-1302, hsa-miR-1304, hsa-miR-1305, hsa-miR-1308, hsa-miR-130a*, hsa-miR-130b*, hsa-miR-132*, hsa-miR-1321, hsa-miR-1324, hsa-miR-135a, hsa-miR-135a*, hsa-miR-135b, hsa-miR-136, hsa-miR-139-5p, hsa-miR-140-3p, hsa-miR-146b-3p, hsa-miR-148b*, hsa-miR-149*, hsa-miR-150*, hsa-miR-151-5p, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-181a, hsa-miR-181b, hsa-miR-181c, hsa-miR-181d, hsa-miR-182, hsa-miR-1826, hsa-miR-183*, hsa-miR-185, hsa-miR-18a*, hsa-miR-194*, hsa-miR-195, hsa-miR-195*, hsa-miR-198, hsa-miR-199a-5p, hsa-miR-199b-5p, hsa-miR-19a*, hsa-miR-19b-1*, hsa-miR-19b-2*, hsa-miR-200a*, hsa-miR-200b, hsa-miR-200b*, hsa-miR-200c, hsa-miR-202*, hsa-miR-203, hsa-miR-204, hsa-miR-205, hsa-miR-206, hsa-miR-20a, hsa-miR-20a*, hsa-miR-20b, hsa-miR-211, hsa-miR-216a, hsa-miR-217, hsa-miR-218-2*, hsa-miR-219-2-3p, hsa-miR-219-5p, hsa-miR-22*, hsa-miR-221, hsa-miR-222, hsa-miR-224, hsa-miR-23 a, hsa-miR-23b, hsa-miR-24, hsa-miR-25, hsa-miR-27a*, hsa-miR-296-5p, hsa-miR-29a, hsa-miR-29a*, hsa-miR-29b, hsa-miR-29c, hsa-miR-302a*, hsa-miR-302f, hsa-miR-30b*, hsa-miR-32, hsa-miR-323-3p, hsa-miR-324-3p, hsa-miR-326, hsa-miR-330-5p, hsa-miR-335*, hsa-miR-338-5p, hsa-miR-339-5p, hsa-miR-33a*, hsa-miR-34a, hsa-miR-342-3p, hsa-miR-342-5p, hsa-miR-345, hsa-miR-34b, hsa-miR-361-3p, hsa-miR-363, hsa-miR-365, hsa-miR-367, hsa-miR-367*, hsa-miR-371-5p, hsa-miR-373*, hsa-miR-374a, hsa-miR-374b, hsa-miR-377, hsa-miR-380, hsa-miR-383, hsa-miR-384, hsa-miR-409-3p, hsa-miR-410, hsa-miR-412, hsa-miR-421, hsa-miR-424, hsa-miR-424*, hsa-miR-425, hsa-miR-429, hsa-miR-431*, hsa-miR-432, hsa-miR-432*, hsa-miR-433, hsa-miR-448, hsa-miR-449, hsa-miR-450b-5p, hsa-miR-452, hsa-miR-453, hsa-miR-455-3p, hsa-miR-485-5p, hsa-miR-486-3p, hsa-miR-489, hsa-miR-490-5p, hsa-miR-493*, hsa-miR-493-5p, hsa-miR-496, hsa-miR-497, hsa-miR-497*, hsa-miR-499-3p, hsa-miR-499-5p, hsa-miR-503, hsa-miR-504, hsa-miR-505*, hsa-miR-510, hsa-miR-513a-3p, hsa-miR-513a-5p, hsa-miR-515-5p, hsa-miR-516a-3p, hsa-miR-516b, hsa-miR-516b*, hsa-miR-518a-5p, hsa-miR-518a-2*, hsa-miR-518c* hsa-miR-519a, hsa-miR-519b-3p, hsa-miR-519c-3p, hsa-miR-519d, hsa-miR-519e*, hsa-miR-520a-5p, hsa-miR-520g, hsa-miR-520h, hsa-miR-522, hsa-miR-525-5p, hsa-miR-526b*, hsa-miR-527, hsa-miR-532-3p, hsa-miR-541, hsa-miR-542-3p, hsa-miR-545, hsa-miR-545*, hsa-miR-548a-3p, hsa-miR-548c-3p, hsa-miR-548e, hsa-miR-548f, hsa-miR-548k, hsa-miR-548l, hsa-miR-548n, hsa-miR-548p, hsa-miR-552, hsa-miR-555, hsa-miR-556-5p, hsa-miR-558, hsa-miR-561, hsa-miR-568, hsa-miR-571, hsa-miR-575, hsa-miR-577, hsa-miR-579, hsa-miR-582-5p, hsa-miR-583, hsa-miR-587, hsa-miR-589*, hsa-miR-590-3p, hsa-miR-593, hsa-miR-595, hsa-miR-596, hsa-miR-605, hsa-miR-606, hsa-miR-607, hsa-miR-612, hsa-miR-613, hsa-miR-616*, hsa-miR-617, hsa-miR-618, hsa-miR-619, hsa-miR-624, hsa-miR-624*, hsa-miR-629, hsa-miR-629*, hsa-miR-633, hsa-miR-636, hsa-miR-640, hsa-miR-641, hsa-miR-642, hsa-miR-643, hsa-miR-646, hsa-miR-648, hsa-miR-650, hsa-miR-651, hsa-miR-653, hsa-miR-654-5p, hsa-miR-655, hsa-miR-656, hsa-miR-657, hsa-miR-660, hsa-miR-664, hsa-miR-668, hsa-miR-7, hsa-miR-708*, hsa-miR-7-1*, hsa-miR-7-2*, hsa-miR-744*, hsa-miR-758, hsa-miR-760, hsa-miR-765, hsa-miR-766, hsa-miR-768-5p, hsa-miR-873, hsa-miR-874, hsa-miR-876-5p, hsa-miR-877*, hsa-miR-888, hsa-miR-888*, hsa-miR-889, hsa-miR-92a, hsa-miR-92b, hsa-miR-93, hsa-miR-940, hsa-miR-96*, and hsa-miR-576-3p.

Bcl-2—BCL2 variant beta—NM_{—}000657—hsa-miR-29a*, hsa-miR-492, and hsa-miR-665.

CXCR4—CXCR4—NM_{—}003467—hsa-miR-1, hsa-miR-1183, hsa-miR-1207-3p, hsa-miR-1264, hsa-miR-1299, hsa-miR-139-5p, hsa-miR-16-2*, hsa-miR-185, hsa-miR-195*, hsa-miR-204, hsa-miR-206, hsa-miR-211, hsa-miR-300, hsa-miR-302c*, has-miR-335, hsa-miR-338-3p, hsa-miR-338-5p, hsa-miR-381, hsa-miR-424*, hsa-miR-452*, hsa-miR-483-3p, hsa-miR-518c*, hsa-miR-545, hsa-miR-548a-3p, hsa-miR-548c-3p, hsa-miR-548d-3p, hsa-miR-548e, hsa-miR-548f, hsa-miR-548g, hsa-miR-548p, hsa-miR-551b*, hsa-miR-588, hsa-miR-613, hsa-miR-634, hsa-miR-655, and hsa-miR-9.

PAPPA—PAPPA—NM_{—}002581—hsa-let-7a, hsa-let-7b, hsa-let-7c, hsa-let-7d, hsa-let-7e, hsa-let-7f, hsa-let-7g, hsa-let-7g*, hsa-let-7i, hsa-miR-103, hsa-miR-105, hsa-miR-107, hsa-miR-1185, hsa-miR-1208, hsa-miR-122, hsa-miR-1224-3p, hsa-miR-1229, hsa-miR-1236, hsa-miR-1237, hsa-miR-1244, hsa-miR-1251, hsa-miR-1252, hsa-miR-1254, hsa-miR-126*, hsa-miR-1261, hsa-miR-1264, hsa-miR-1266, hsa-miR-1269, hsa-miR-1271, hsa-miR-1272, hsa-miR-1274a, hsa-miR-1275, hsa-miR-1278, hsa-miR-1287, hsa-miR-1288, hsa-miR-1294, hsa-miR-1296, hsa-miR-1297, hsa-miR-1298, hsa-miR-1300, hsa-miR-1301, hsa-miR-1303, hsa-miR-1305, hsa-miR-1306, hsa-miR-130b*, hsa-miR-1321, hsa-miR-1323, hsa-miR-1324, hsa-miR-133a, hsa-miR-133b, hsa-miR-138, hsa-miR-141, hsa-miR-142-5p, hsa-miR-143, hsa-miR-143*, hsa-miR-144, hsa-miR-145, -146a, hsa-miR-146b, hsa-miR-147, hsa-miR-149*, hsa-miR-150, hsa-miR-150*, hsa-miR-154, hsa-miR-155*, hsa-miR-15a, hsa-miR-15a*, hsa-miR-15b, hsa-miR-16, hsa-miR-181a-2*, hsa-miR-182, hsa-miR-1825, hsa-miR-1826, hsa-miR-185, hsa-miR-185*, hsa-miR-186, hsa-miR-186*, hsa-miR-18a, hsa-miR-18a*, hsa-miR-18b, hsa-miR-190, hsa-miR-190b, hsa-miR-192*, hsa-miR-193a-5p, hsa-miR-194*, hsa-miR-195, hsa-miR-196a, hsa-miR-196b, hsa-miR-199a-3p, hsa-miR-199b-3p, hsa-miR-200a, hsa-miR-203, hsa-miR-20a*, hsa-miR-21*, hsa-miR-214, hsa-miR-216b, hsa-miR-217, hsa-miR-218-1*, hsa-miR-219-1-3p, hsa-miR-219-2-3p, hsa-miR-22, hsa-miR-22*, hsa-miR-23a*, hsa-miR-23b*, hsa-miR-24-1*, hsa-miR-24-2*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-28-5p, hsa-miR-296-3p, hsa-miR-298, hsa-miR-299-3p, hsa-miR-29a*, hsa-miR-300, hsa-miR-302a, hsa-miR-302b, hsa-miR-302c, hsa-miR-302d, hsa-miR-302e, hsa-miR-30a*, hsa-miR-30b*, hsa-miR-30c-1*, hsa-miR-30c-2*, hsa-miR-30d*, hsa-miR-30e*, hsa-miR-31, hsa-miR-326, hsa-miR-328, hsa-miR-330-5p, hsa-miR-331-5p, hsa-miR-335*, hsa-miR-338-3p, hsa-miR-338-5p, hsa-miR-339-5p, hsa-miR-33a, hsa-miR-33b, hsa-miR-340, hsa-miR-342-3p, hsa-miR-34a, hsa-miR-34a*, hsa-miR-34b*, hsa-miR-34c-5p, hsa-miR-362-5p, hsa-miR-365, hsa-miR-369-3p, hsa-miR-372, hsa-miR-373, hsa-miR-373*, hsa-miR-374a, hsa-miR-374b, hsa-miR-374b*, hsa-miR-376a, hsa-miR-376b, hsa-miR-376c, hsa-miR-377, hsa-miR-377*, hsa-miR-378, hsa-miR-379*, hsa-miR-381, hsa-miR-383, hsa-miR-384, hsa-miR-411, hsa-miR-411*, hsa-miR-421, hsa-miR-422a, hsa-miR-424, hsa-miR-432*, hsa-miR-448, hsa-miR-449a, hsa-miR-449b, hsa-miR-452, hsa-miR-485-5p, hsa-miR-489, hsa-miR-490-3p, hsa-miR-494, hsa-miR-496, hsa-miR-497, hsa-miR-500, hsa-miR-501-5p, hsa-miR-503, hsa-miR-507, hsa-miR-509-3p, hsa-miR-510, hsa-miR-511, hsa-miR-512-3p, hsa-miR-513a-3p, hsa-miR-513a-5p, hsa-miR-513c, hsa-miR-515-5p, hsa-miR-516a-3p, hsa-miR-516b*, hsa-miR-517*, hsa-miR-517b, hsa-miR-518c*, hsa-miR-519e*, hsa-miR-519f*, hsa-miR-520a-3p, hsa-miR-520b, hsa-miR-520c-3p, hsa-miR-520d-3p, hsa-miR-520e, hsa-miR-520g, hsa-miR-520h, hsa-miR-522, hsa-miR-526a, hsa-miR-526b, hsa-miR-526c, hsa-miR-532-3p, hsa-miR-532-5p, hsa-miR-541*, hsa-miR-544, hsa-miR-545, hsa-miR-548a-3p, hsa-miR-548d-3p, hsa-miR-548e, hsa-miR-548f, hsa-miR-548g, hsa-miR-548l, hsa-miR-548m, hsa-miR-548n, hsa-miR-548o, hsa-miR-548p, hsa-miR-549, hsa-miR-551b*, hsa-miR-555, hsa-miR-556-3p, hsa-miR-557, hsa-miR-558, hsa-miR-561, hsa-miR-562, hsa-miR-570, hsa-miR-571, hsa-miR-574-5p, hsa-miR-576-5p, hsa-miR-578, hsa-miR-579, hsa-miR-582-3p, hsa-miR-583, hsa-miR-584, hsa-miR-586, hsa-miR-588, hsa-miR-589, hsa-miR-590-3p, hsa-miR-591, hsa-miR-592, hsa-miR-593, hsa-miR-599, hsa-miR-600, hsa-miR-605, hsa-miR-616*, hsa-miR-619, hsa-miR-625, hsa-miR-626, hsa-miR-627, hsa-miR-628-5p, hsa-miR-629, hsa-miR-629*, hsa-miR-637, hsa-miR-646, hsa-miR-649, hsa-miR-651, hsa-miR-653, hsa-miR-654-3p, hsa-miR-656, hsa-miR-659, hsa-miR-664, hsa-miR-665, hsa-miR-708, hsa-miR-7-1*, hsa-miR-7-2*, hsa-miR-760, hsa-miR-767-3p, hsa-miR-768-3p, hsa-miR-768-5p, hsa-miR-875-3p, hsa-miR-876-3p, hsa-miR-877, hsa-miR-877*, hsa-miR-889, hsa-miR-892b, hsa-miR-922, hsa-miR-92a-1*, hsa-miR-92a-2*, hsa-miR-93*, hsa-miR-936, hsa-miR-942, hsa-miR-943, hsa-miR-944, hsa-miR-96, and hsa-miR-98.

Tight Junction Protein 2—TJP2 variant 1—NM_{—}004817—hsa-miR-105, hsa-miR-106a*, hsa-miR-1197, hsa-miR-1224-5p, hsa-miR-1226, hsa-miR-1233, hsa-miR-1234, hsa-miR-124, hsa-miR-1253, hsa-miR-1260, hsa-miR-128, hsa-miR-1289, hsa-miR-1297, hsa-miR-130a*, hsa-miR-132, hsa-miR-137, hsa-miR-142-5p, hsa-miR-144, hsa-miR-148a, hsa-miR-148b, hsa-miR-148b*, hsa-miR-152, hsa-miR-16-1*, hsa-miR-17*, hsa-miR-181a-2*, hsa-miR-188-3p, hsa-miR-194, hsa-miR-212, hsa-miR-218, hsa-miR-221*, hsa-miR-23a, hsa-miR-23b, hsa-miR-26a, hsa-miR-26b, hsa-miR-30c-1*, hsa-miR-30c-2*, hsa-miR-32*, hsa-miR-331-3p, hsa-miR-33a*, hsa-miR-342-3p, hsa-miR-345, hsa-miR-425, hsa-miR-455-3p, hsa-miR-495, hsa-miR-498, hsa-miR-506, hsa-miR-507, hsa-miR-509-3-5p, hsa-miR-509-5p, hsa-miR-513a-3p, hsa-miR-520d-5p, hsa-miR-524-5p, hsa-miR-532-5p, hsa-miR-548a-3p, hsa-miR-548b-3p, hsa-miR-548e, hsa-miR-548f, hsa-miR-548g, hsa-miR-548k, hsa-miR-557, hsa-miR-629, hsa-miR-635, hsa-miR-661, hsa-miR-7-1*, hsa-miR-7-2*, hsa-miR-760, hsa-miR-802, hsa-miR-9*, and hsa-miR-940.

TJP2 variant 2—NM_{—}201629—hsa-miR-105, hsa-miR-106a*, hsa-miR-1197, hsa-miR-1224-5p, hsa-miR-1226, hsa-miR-1233, hsa-miR-1234, hsa-miR-124, hsa-miR-1253, hsa-miR-1260, hsa-miR-128, hsa-miR-1289, hsa-miR-1297, hsa-miR-130a*, hsa-miR-132, hsa-miR-137, hsa-miR-142-5p, hsa-miR-144, hsa-miR-148a, hsa-miR-148b, hsa-miR-148b*, hsa-miR-152, hsa-miR-16-1*, hsa-miR-17*, hsa-miR-181a-2*, hsa-miR-188-3p, hsa-miR-194, hsa-miR-212, hsa-miR-218, hsa-miR-221*, hsa-miR-23a, hsa-miR-23b, hsa-miR-26a, hsa-miR-26b, hsa-miR-30c-1*, hsa-miR-30c-2*, hsa-miR-32*, hsa-miR-331-3p, hsa-miR-33a*, hsa-miR-342-3p, hsa-miR-345, hsa-miR-425, hsa-miR-455-3p, hsa-miR-495, hsa-miR-498, hsa-miR-506, hsa-miR-507, hsa-miR-509-3-5p, hsa-miR-509-5p, hsa-miR-513a-3p, hsa-miR-520d-5p, hsa-miR-524-5p, hsa-miR-532-5p, hsa-miR-548a-3p, hsa-miR-548b-3p, hsa-miR-548e, hsa-miR-548f, hsa-miR-548g, hsa-miR-548k, hsa-miR-557, hsa-miR-629, hsa-miR-635, hsa-miR-661, hsa-miR-7-1*, hsa-miR-7-2*, hsa-miR-760, hsa-miR-802, hsa-miR-9*, and hsa-miR-940.

Abl-interactor 1—ABI1 variant 1—NM_{—}005470—hsa-abi-13197, hsa-let-7a*, hsa-let-7b*, hsa-let-7f-1*, hsa-miR-106a, hsa-miR-106b, hsa-miR-1208, hsa-miR-1224-3p, hsa-miR-1226, hsa-miR-1246, hsa-miR-1248, hsa-miR-1252, hsa-miR-1279, hsa-miR-129-5p, hsa-miR-1302, hsa-miR-130a*, hsa-miR-138-1*, hsa-miR-142-5p, hsa-miR-144, hsa-miR-148b*, hsa-miR-150*, hsa-miR-154, hsa-miR-15a*, hsa-miR-15b*, hsa-miR-16-1*, hsa-miR-17, hsa-miR-181a, hsa-miR-181b, hsa-miR-181c, hsa-miR-181d, hsa-miR-188-3p, hsa-miR-199a-5p, hsa-miR-199b-5p, hsa-miR-19a*, hsa-miR-19b-1*, hsa-miR-19b-2*, hsa-miR-200c*, hsa-miR-20a, hsa-miR-20a*, hsa-miR-20b, hsa-miR-216b, hsa-miR-222*, hsa-miR-23a, hsa-miR-23b, hsa-miR-24-1*, hsa-miR-24-2*, hsa-miR-29a, hsa-miR-29a*, hsa-miR-29b, hsa-miR-29c, hsa-miR-302c*, hsa-miR-302f, hsa-miR-32*, hsa-miR-323-3p, hsa-miR-337-3p, hsa-miR-338-3p, hsa-miR-338-5p, hsa-miR-33a, hsa-miR-33b, hsa-miR-33b*, hsa-miR-34c-3p, hsa-miR-361-3p, hsa-miR-379, hsa-miR-380, hsa-miR-409-3p, hsa-miR-432, hsa-miR-433, hsa-miR-448, hsa-miR-452, hsa-miR-491-3p, hsa-miR-493*, hsa-miR-494, hsa-miR-495, hsa-miR-497*, hsa-miR-500, hsa-miR-507, hsa-miR-513a-3p, hsa-miR-514, hsa-miR-515-3p, hsa-miR-519a, hsa-miR-519b-3p, hsa-miR-519c-3p, hsa-miR-519d, hsa-miR-519e, hsa-miR-520g, hsa-miR-520h, hsa-miR-522, hsa-miR-526b*, hsa-miR-544, hsa-miR-548c-3p, hsa-miR-548d-3p, hsa-miR-548m, hsa-miR-550*, hsa-miR-55 1 b*, hsa-miR-554, hsa-miR-557, hsa-miR-561, hsa-miR-568, hsa-miR-570, hsa-miR-576-5p, hsa-miR-578, hsa-miR-590-3p, hsa-miR-600, hsa-miR-607, hsa-miR-633, hsa-miR-634, hsa-miR-643, hsa-miR-655, hsa-miR-656, hsa-miR-659, hsa-miR-7-1*, hsa-miR-7-2*, hsa-miR-769-5p, hsa-miR-770-5p, hsa-miR-885-5p, hsa-miR-888*, hsa-miR-889, hsa-miR-9, hsa-miR-93, hsa-miR-936, and hsa-miR-944.

ABI1 variant 2—NM_{—}001012750—hsa-abi-13197, hsa-let-7a*, hsa-let-7b*, hsa-let-7f-1*, hsa-miR-106a, hsa-miR-106b, hsa-miR-1208, hsa-miR-1224-3p, hsa-miR-1226, hsa-miR-1246, hsa-miR-1248, hsa-miR-1252, hsa-miR-1279, hsa-miR-129-5p, hsa-miR-1302, hsa-miR-130a*, hsa-miR-138-1*, hsa-miR-142-5p, hsa-miR-144, hsa-miR-148b*, hsa-miR-150*, hsa-miR-154, hsa-miR-15a*, hsa-miR-15b*, hsa-miR-16-1*, hsa-miR-17, hsa-miR-181a, hsa-miR-181b, hsa-miR-181c, hsa-miR-181d, hsa-miR-188-3p, hsa-miR-199a-5p, hsa-miR-199b-5p, hsa-miR-19a*, hsa-miR-19b-1*, hsa-miR-19b-2*, hsa-miR-200c*, hsa-miR-20a, hsa-miR-20a*, hsa-miR-20b, hsa-miR-216b, hsa-miR-222*, hsa-miR-23a, hsa-miR-23b, hsa-miR-24-1*, hsa-miR-24-2*, hsa-miR-29a, hsa-miR-29a*, hsa-miR-29b, hsa-miR-29c, hsa-miR-302c*, hsa-miR-302f, hsa-miR-32*, hsa-miR-323-3p, hsa-miR-337-3p, hsa-miR-338-3p, hsa-miR-338-5p, hsa-miR-33a, hsa-miR-33b, hsa-miR-33b*, hsa-miR-34c-3p, hsa-miR-361-3p, hsa-miR-379, hsa-miR-380, hsa-miR-409-3p, hsa-miR-432, hsa-miR-433, hsa-miR-448, hsa-miR-452, hsa-miR-491-3p, hsa-miR-493*, hsa-miR-494, hsa-miR-495, hsa-miR-497*, hsa-miR-500, hsa-miR-507, hsa-miR-513a-3p, hsa-miR-514, hsa-miR-515-3p, hsa-miR-519a, hsa-miR-519b-3p, hsa-miR-519c-3p, hsa-miR-519d, hsa-miR-519e, hsa-miR-520g, hsa-miR-520h, hsa-miR-522, hsa-miR-526b*, hsa-miR-544, hsa-miR-548c-3p, hsa-miR-548d-3p, hsa-miR-548m, hsa-miR-550*, hsa-miR-551b*, hsa-miR-554, hsa-miR-557, hsa-miR-561, hsa-miR-568, hsa-miR-570, hsa-miR-576-5p, hsa-miR-578, hsa-miR-590-3p, hsa-miR-600, hsa-miR-607, hsa-miR-633, hsa-miR-634, hsa-miR-643, hsa-miR-655, hsa-miR-656, hsa-miR-659, hsa-miR-7-1*, hsa-miR-7-2*, hsa-miR-769-5p, hsa-miR-770-5p, hsa-miR-885-5p, hsa-miR-888*, hsa-miR-889, hsa-miR-9, hsa-miR-93, hsa-miR-936, and hsa-miR-944.

ABI1 variant 3—NM_{—}001012751—hsa-abi-13197, hsa-let-7a*, hsa-let-7b*, hsa-let-7f-1*, hsa-miR-106a, hsa-miR-106b, hsa-miR-1208, hsa-miR-1224-3p, hsa-miR-1226, hsa-miR-1246, hsa-miR-1248, hsa-miR-1252, hsa-miR-1279, hsa-miR-129-5p, hsa-miR-1302, hsa-miR-130a*, hsa-miR-138-1*, hsa-miR-142-5p, hsa-miR-144, hsa-miR-148b*, hsa-miR-150*, hsa-miR-154, hsa-miR-15a*, hsa-miR-15b*, hsa-miR-16-1*, hsa-miR-17, hsa-miR-181a, hsa-miR-181b, hsa-miR-181c, hsa-miR-181d, hsa-miR-188-3p, hsa-miR-199a-5p, hsa-miR-199b-5p, hsa-miR-19a*, hsa-miR-19b-1*, hsa-miR-19b-2*, hsa-miR-200c*, hsa-miR-20a, hsa-miR-20a*, hsa-miR-20b, hsa-miR-216b, hsa-miR-222*, hsa-miR-23 a, hsa-miR-23b, hsa-miR-24-1*, hsa-miR-24-2*, hsa-miR-29a, hsa-miR-29a*, hsa-miR-29b, hsa-miR-29c, hsa-miR-302c*, hsa-miR-302f, hsa-miR-32*, hsa-miR-323-3p, hsa-miR-337-3p, hsa-miR-338-3p, hsa-miR-338-5p, hsa-miR-33a, hsa-miR-33b, hsa-miR-33b*, hsa-miR-34c-3p, hsa-miR-361-3p, hsa-miR-379, hsa-miR-380, hsa-miR-409-3p, hsa-miR-432, hsa-miR-433, hsa-miR-448, hsa-miR-452, hsa-miR-491-3p, hsa-miR-493*, hsa-miR-494, hsa-miR-495, hsa-miR-497*, hsa-miR-500, hsa-miR-507, hsa-miR-513a-3p, hsa-miR-514, hsa-miR-515-3p, hsa-miR-519a, hsa-miR-519b-3p, hsa-miR-519c-3p, hsa-miR-519d, hsa-miR-519e, hsa-miR-520g, hsa-miR-520h, hsa-miR-522, hsa-miR-526b*, hsa-miR-544, hsa-miR-548c-3p, hsa-miR-548d-3p, hsa-miR-548m, hsa-miR-550*, hsa-miR-551b*, hsa-miR-554, hsa-miR-557, hsa-miR-561, hsa-miR-568, hsa-miR-570, hsa-miR-576-5p, hsa-miR-578, hsa-miR-590-3p, hsa-miR-600, hsa-miR-607, hsa-miR-633, hsa-miR-634, hsa-miR-643, hsa-miR-655, hsa-miR-656, hsa-miR-659, hsa-miR-7-1*, hsa-miR-7-2*, hsa-miR-769-5p, hsa-miR-770-5p, hsa-miR-885-5p, hsa-miR-888*, hsa-miR-889, hsa-miR-9, hsa-miR-93, hsa-miR-936, and hsa-miR-944.

ABI1 variant 4—NM_{—}001012752—hsa-abi-13197, hsa-let-7a*, hsa-let-7b*, hsa-let-7f-1*, hsa-miR-106a, hsa-miR-106b, hsa-miR-1208, hsa-miR-1224-3p, hsa-miR-1226, hsa-miR-1246, hsa-miR-1248, hsa-miR-1252, hsa-miR-1279, hsa-miR-129-5p, hsa-miR-1302, hsa-miR-130a*, hsa-miR-138-1*, hsa-miR-142-5p, hsa-miR-144, hsa-miR-148b*, hsa-miR-150*, hsa-miR-154, hsa-miR-15a*, hsa-miR-15b*, hsa-miR-16-1*, hsa-miR-17, hsa-miR-181a, hsa-miR-181b, hsa-miR-181c, hsa-miR-181d, hsa-miR-188-3p, hsa-miR-199a-5p, hsa-miR-199b-5p, hsa-miR-19a*, hsa-miR-19b-1*, hsa-miR-19b-2*, hsa-miR-200c*, hsa-miR-20a, hsa-miR-20a*, hsa-miR-20b, hsa-miR-216b, hsa-miR-222*, hsa-miR-23a, hsa-miR-23b, hsa-miR-24-1*, hsa-miR-24-2*, hsa-miR-29a, hsa-miR-29a*, hsa-miR-29b, hsa-miR-29c, hsa-miR-302c*, hsa-miR-302f, hsa-miR-32*, hsa-miR-323-3p, hsa-miR-337-3p, hsa-miR-338-3p, hsa-miR-338-5p, hsa-miR-33a, hsa-miR-33b, hsa-miR-33b*, hsa-miR-34c-3p, hsa-miR-361-3p, hsa-miR-379, hsa-miR-380, hsa-miR-409-3p, hsa-miR-432, hsa-miR-433, hsa-miR-448, hsa-miR-452, hsa-miR-491-3p, hsa-miR-493*, hsa-miR-494, hsa-miR-495, hsa-miR-497*, hsa-miR-500, hsa-miR-507, hsa-miR-513a-3p, hsa-miR-514, hsa-miR-515-3p, hsa-miR-519a, hsa-miR-519b-3p, hsa-miR-519c-3p, hsa-miR-519d, hsa-miR-519e, hsa-miR-520g, hsa-miR-520h, hsa-miR-522, hsa-miR-526b*, hsa-miR-544, hsa-miR-548c-3p, hsa-miR-548d-3p, hsa-miR-548m, hsa-miR-550*, hsa-miR-551b*, hsa-miR-554, hsa-miR-557, hsa-miR-561, hsa-miR-568, hsa-miR-570, hsa-miR-576-5p, hsa-miR-578, hsa-miR-590-3p, hsa-miR-600, hsa-miR-607, hsa-miR-633, hsa-miR-634, hsa-miR-643, hsa-miR-655, hsa-miR-656, hsa-miR-659, hsa-miR-7-1*, hsa-miR-7-2*, hsa-miR-769-5p, hsa-miR-770-5p, hsa-miR-885-5p, hsa-miR-888*, hsa-miR-889, hsa-miR-9, hsa-miR-93, hsa-miR-936, and hsa-miR-944.

B-cell translocation gene 1—BTG1—NM_{—}001731—hsa-abi-10411, hsa-abi-10617, hsa-abi-13182, hsa-abi-13209, hsa-abi-13242, hsa-abi-13255, hsa-abi-13268, hsa-abi-3046, hsa-abi-9508, hsa-let-7g*, hsa-miR-10b*, hsa-miR-1182, hsa-miR-1231, hsa-miR-1245, hsa-miR-1253, hsa-miR-1264, hsa-miR-130a, hsa-miR-130b, hsa-miR-138, hsa-miR-140-3p, hsa-miR-142-5p, hsa-miR-183, hsa-miR-186*, hsa-miR-193a-5p, hsa-miR-19a, hsa-miR-19b, hsa-miR-21*, hsa-miR-22, hsa-miR-27a, hsa-miR-27b, hsa-miR-300, hsa-miR-301a, hsa-miR-301b, hsa-miR-302a, hsa-miR-302b, hsa-miR-302c, hsa-miR-302d, hsa-miR-302e, hsa-miR-30c-1*, hsa-miR-30c-2*, hsa-miR-32*, hsa-miR-320a, hsa-miR-320b, hsa-miR-320c, hsa-miR-320d, hsa-miR-330-3p, hsa-miR-331-5p, hsa-miR-367*, hsa-miR-372, hsa-miR-373, hsa-miR-381, hsa-miR-424*, hsa-miR-454, hsa-miR-488, hsa-miR-493*, hsa-miR-497*, hsa-miR-498, hsa-miR-509-3-5p, hsa-miR-509-5p, hsa-miR-511, hsa-miR-512-3p, hsa-miR-513a-3p, hsa-miR-513a-5p, hsa-miR-520a-3p, hsa-miR-520b, hsa-miR-520c-3p, hsa-miR-520d-3p, hsa-miR-520e, hsa-miR-520f, hsa-miR-539, hsa-miR-541*, hsa-miR-548c-3p, hsa-miR-548m, hsa-miR-548p, hsa-miR-551b*, hsa-miR-565, hsa-miR-581, hsa-miR-587, hsa-miR-617, hsa-miR-621, hsa-miR-624, hsa-miR-633, hsa-miR-640, hsa-miR-654-3p, hsa-miR-744*, hsa-miR-770-5p, hsa-miR-876-3p, hsa-miR-890, and hsa-miR-93.

Interleukin 6—IL6—NM_{—}000600—hsa-let-7a, hsa-let-7b, hsa-let-7c, hsa-let-7e, hsa-let-7g, hsa-let-7i, hsa-miR-1252, hsa-miR-126*, hsa-miR-1323, hsa-miR-146a*, hsa-miR-148a*, hsa-miR-148b*, hsa-miR-149, hsa-miR-153, hsa-miR-217, hsa-miR-338-5p, hsa-miR-33a*, hsa-miR-365, hsa-miR-512-5p, hsa-miR-548d-3p, hsa-miR-548o, hsa-miR-568, hsa-miR-574-3p, hsa-miR-613, hsa-miR-655, hsa-miR-760, and hsa-miR-98.

CASP8 and FADD-like apoptosis regulator—CFLAR variant 1—NM_{—}003879—hsa-miR-106a, hsa-miR-106b, hsa-miR-1224-3p, hsa-miR-1245, hsa-miR-1260, hsa-miR-150, hsa-miR-17, hsa-miR-186*, hsa-miR-18a*, hsa-miR-20a, hsa-miR-20b, hsa-miR-29b-2*, hsa-miR-302a, hsa-miR-302b, hsa-miR-302c, hsa-miR-302d, hsa-miR-302e, hsa-miR-372, hsa-miR-373, hsa-miR-378*, hsa-miR-450b-3p, hsa-miR-504, hsa-miR-512-3p, hsa-miR-519d, hsa-miR-520a-3p, hsa-miR-520b, hsa-miR-520c-3p, hsa-miR-520d-3p, hsa-miR-520e, hsa-miR-520g, hsa-miR-520h, hsa-miR-526b*, hsa-miR-532-3p, hsa-miR-552, hsa-miR-573, hsa-miR-769-3p, and hsa-miR-93.

CFLAR variant 2—NM_{—}001127183—hsa-miR-106a, hsa-miR-106b, hsa-miR-1245, hsa-miR-150, hsa-miR-17, hsa-miR-186*, hsa-miR-18a*, hsa-miR-20a, hsa-miR-20b, hsa-miR-29b-2*, hsa-miR-302a, hsa-miR-302b, hsa-miR-302c, hsa-miR-302d, hsa-miR-302e, hsa-miR-372, hsa-miR-373, hsa-miR-378*, hsa-miR-512-3p, hsa-miR-519d, hsa-miR-520a-3p, hsa-miR-520b, hsa-miR-520c-3p, hsa-miR-520d-3p, hsa-miR-520e, hsa-miR-520g, hsa-miR-520h, hsa-miR-526b*, hsa-miR-552, hsa-miR-573, and hsa-miR-93.

CFLAR variant 3—NM_{—}001127184—hsa-miR-126*, hsa-miR-1827, hsa-miR-34b*, and hsa-miR-802.

Smu-1 suppressor of mec-8 and unc-52 homolog—SMU1—NM_{—}018225—hsa-miR-101, hsa-miR-103, hsa-miR-105, hsa-miR-106a, hsa-miR-106b, hsa-miR-107, hsa-miR-10a, hsa-miR-10b, hsa-miR-1184, hsa-miR-1202, hsa-miR-1203, hsa-miR-1205, hsa-miR-1208, hsa-miR-122, hsa-miR-1224-3p, hsa-miR-1226*, hsa-miR-1227, hsa-miR-1228, hsa-miR-124, hsa-miR-1246, hsa-miR-1251, hsa-miR-1252, hsa-miR-1257, hsa-miR-1259, hsa-miR-126*, hsa-miR-1260, hsa-miR-1264, hsa-miR-1267, hsa-miR-1269, hsa-miR-127-3p, hsa-miR-1274a, hsa-miR-127-5p, hsa-miR-1278, hsa-miR-128, hsa-miR-1284, hsa-miR-1285, hsa-miR-1289, hsa-miR-129*, hsa-miR-1291, hsa-miR-1293, hsa-miR-129-3p, hsa-miR-1299, hsa-miR-1300, hsa-miR-1304, hsa-miR-1305, hsa-miR-1322, hsa-miR-1323, hsa-miR-134, hsa-miR-135b*, hsa-miR-140-5p, hsa-miR-141, hsa-miR-141*, hsa-miR-142-5p, hsa-miR-143*, hsa-miR-146a*, hsa-miR-148a, hsa-miR-148b, hsa-miR-149*, hsa-miR-150, hsa-miR-151-5p, hsa-miR-152, hsa-miR-154*, hsa-miR-155*, hsa-miR-15a, hsa-miR-15b, hsa-miR-16, hsa-miR-16-1*, hsa-miR-16-2*, hsa-miR-17, hsa-miR-181a-2*, hsa-miR-1825, hsa-miR-1827, hsa-miR-183, hsa-miR-186*, hsa-miR-187, hsa-miR-188-3p, hsa-miR-194, hsa-miR-195, hsa-miR-195*, hsa-miR-196a, hsa-miR-196a*, hsa-miR-196b, hsa-miR-19a, hsa-miR-19b, hsa-miR-200a, hsa-miR-203, hsa-miR-205, hsa-miR-208a, hsa-miR-208b, hsa-miR-20a, hsa-miR-20a*, hsa-miR-20b, hsa-miR-20b*, hsa-miR-216a, hsa-miR-217, hsa-miR-218, hsa-miR-219-1-3p, hsa-miR-219-2-3p, hsa-miR-220b, hsa-miR-23a*, hsa-miR-23b*, hsa-miR-25, hsa-miR-25*, hsa-miR-26a-1*, hsa-miR-26a-2*, hsa-miR-26b*, hsa-miR-28-3p, hsa-miR-296-5p, hsa-miR-29a*, hsa-miR-29b-1*, hsa-miR-300, hsa-miR-302a, hsa-miR-302a*, hsa-miR-302b, hsa-miR-302c, hsa-miR-302c*, hsa-miR-302d, hsa-miR-302e, hsa-miR-30b*, hsa-miR-31, hsa-miR-32, hsa-miR-320a, hsa-miR-320b, hsa-miR-320c, hsa-miR-320d, hsa-miR-323-3p, hsa-miR-323-5p, hsa-miR-324-3p, hsa-miR-337-5p, hsa-miR-338-5p, hsa-miR-33a, hsa-miR-33a*, hsa-miR-33b, hsa-miR-340, hsa-miR-34a, hsa-miR-34c-5p, hsa-miR-363, hsa-miR-363*, hsa-miR-367, hsa-miR-369-3p, hsa-miR-372, hsa-miR-373, hsa-miR-373*, hsa-miR-374a*, hsa-miR-376a*, hsa-miR-377*, hsa-miR-378*, hsa-miR-380, hsa-miR-381, hsa-miR-382, hsa-miR-384, hsa-miR-409-5p, hsa-miR-424, hsa-miR-431, hsa-miR-449a, hsa-miR-449b, hsa-miR-452, hsa-miR-455-5p, hsa-miR-485-5p, hsa-miR-487a, hsa-miR-489, hsa-miR-490-3p, hsa-miR-490-5p, hsa-miR-493*, hsa-miR-494, hsa-miR-497, hsa-miR-500*, hsa-miR-501-5p, hsa-miR-503, hsa-miR-506, hsa-miR-507, hsa-miR-508-5p, hsa-miR-511, hsa-miR-512-3p, hsa-miR-513c, hsa-miR-517a, hsa-miR-517c, hsa-miR-519d, hsa-miR-520a-3p, hsa-miR-520b, hsa-miR-520c-3p, hsa-miR-520d-3p, hsa-miR-520d-5p, hsa-miR-520e, hsa-miR-520g, hsa-miR-520h, hsa-miR-524-5p, hsa-miR-526b*, hsa-miR-532-3p, hsa-miR-542-3p, hsa-miR-545, hsa-miR-548a-5p, hsa-miR-548b-3p, hsa-miR-548b-5p, hsa-miR-548c-3p, hsa-miR-548c-5p, hsa-miR-548d-3p, hsa-miR-548d-5p, hsa-miR-548g, hsa-miR-548h, hsa-miR-548i, hsa-miR-548j, hsa-miR-548k, hsa-miR-548m, hsa-miR-548n, hsa-miR-548o, hsa-miR-548p, hsa-miR-551b*, hsa-miR-553, hsa-miR-554, hsa-miR-557, hsa-miR-559, hsa-miR-561, hsa-miR-568, hsa-miR-570, hsa-miR-577, hsa-miR-578, hsa-miR-580, hsa-miR-582-3p, hsa-miR-583, hsa-miR-586, hsa-miR-589, hsa-miR-589*, hsa-miR-590-3p, hsa-miR-593*, hsa-miR-597, hsa-miR-607, hsa-miR-612, hsa-miR-616*, hsa-miR-619, hsa-miR-624*, hsa-miR-628-5p, hsa-miR-630, hsa-miR-633, hsa-miR-640, hsa-miR-643, hsa-miR-647, hsa-miR-649, hsa-miR-653, hsa-miR-655, hsa-miR-657, hsa-miR-658, hsa-miR-661, hsa-miR-671-5p, hsa-miR-708*, hsa-miR-766, hsa-miR-875-3p, hsa-miR-875-5p, hsa-miR-876-5p, hsa-miR-885-5p, hsa-miR-886-3p, hsa-miR-888*, hsa-miR-890, hsa-miR-920, hsa-miR-922, hsa-miR-924, hsa-miR-92a, hsa-miR-92b, hsa-miR-92b*, hsa-miR-93, hsa-miR-933, hsa-miR-937, hsa-miR-938, hsa-miR-939, hsa-miR-940, hsa-miR-942, hsa-miR-943, and hsa-miR-944.

S100 calcium binding protein A3—S100A3—NM_{—}002960—hsa-miR-1200, hsa-miR-1233, hsa-miR-1281, hsa-miR-1285, hsa-miR-1298, hsa-miR-1321, hsa-miR-185, hsa-miR-198, hsa-miR-220c, hsa-miR-296-5p, hsa-miR-298, hsa-miR-30c-1*, hsa-miR-30c-2*, hsa-miR-326, hsa-miR-330-5p, hsa-miR-331-3p, hsa-miR-342-5p, hsa-miR-488, hsa-miR-612, hsa-miR-631, hsa-miR-675, hsa-miR-93*, and hsa-miR-940.

Ras and EF-hand domain containing—RASEF—NM_{—}152573—hsa-miR-1227, hsa-miR-1265, hsa-miR-1299, hsa-miR-16-1*, hsa-miR-222*, hsa-miR-224, hsa-miR-34b, hsa-miR-377, hsa-miR-455-5p, hsa-miR-492, hsa-miR-513a-5p, hsa-miR-520a-5p, hsa-miR-525-5p, hsa-miR-630, and hsa-miR-875-3p.

Interferon gamma receptor 1—IFNGR1—NM_{—}000416—hsa-miR-100*, hsa-miR-1178, hsa-miR-1243, hsa-miR-1278, hsa-miR-1283, hsa-miR-136, hsa-miR-183*, hsa-miR-187*, hsa-miR-216b, hsa-miR-224, hsa-miR-335*, hsa-miR-33a*, hsa-miR-520d-5p, hsa-miR-524-5p, hsa-miR-548a-3p, hsa-miR-548c-3p, hsa-miR-548e, hsa-miR-548f, hsa-miR-548g, hsa-miR-573, hsa-miR-665, hsa-miR-668, hsa-miR-767-3p, and hsa-miR-891b.

Insulin growth factor-like family member 1—IGFL1—NM_{—}198541—hsa-miR-1205, hsa-miR-1226, hsa-miR-1266, hsa-miR-1293, hsa-miR-130b*, hsa-miR-1324, hsa-miR-142-5p, hsa-miR-143, hsa-miR-146a, hsa-miR-146b-5p, hsa-miR-198, hsa-miR-21*, hsa-miR-216a, hsa-miR-218, hsa-miR-23a*, hsa-miR-23b*, hsa-miR-296-5p, hsa-miR-324-5p, hsa-miR-335*, hsa-miR-340, hsa-miR-492, hsa-miR-512-5p, hsa-miR-541, hsa-miR-548k, hsa-miR-573, hsa-miR-574-5p, hsa-miR-608, hsa-miR-654-5p, and hsa-miR-665.

Microseminoprotein, beta-(PSP94)—MSMB—NM_{—}002443—hsa-miR-1197, hsa-miR-1233, hsa-miR-146b-3p, hsa-miR-223*, hsa-miR-24-1*, hsa-miR-24-2*, hsa-miR-33a*, hsa-miR-373*, hsa-miR-548c-3p, hsa-miR-551b*, hsa-miR-616*, hsa-miR-630, and hsa-miR-661.

CEACAM5—CEACAM5—NM_{—}004363—hsa-miR-105, hsa-miR-106a, hsa-miR-106b, hsa-miR-1183, hsa-miR-1184, hsa-miR-1200, hsa-miR-1206, hsa-miR-1264, hsa-miR-127-5p, hsa-miR-1276, hsa-miR-1307, hsa-miR-1324, hsa-miR-134, hsa-miR-135a*, hsa-miR-143*, hsa-miR-148a, hsa-miR-148b, hsa-miR-151-3p, hsa-miR-152, hsa-miR-17, hsa-miR-186, hsa-miR-193b*, hsa-miR-203, hsa-miR-20a, hsa-miR-20b, hsa-miR-216a, hsa-miR-220b, hsa-miR-223, hsa-miR-302a, hsa-miR-302b, hsa-miR-302c, hsa-miR-302d, hsa-miR-302e, hsa-miR-302f, hsa-miR-335*, hsa-miR-338-5p, hsa-miR-33a*, hsa-miR-34a*, hsa-miR-361-5p, hsa-miR-362-5p, hsa-miR-367*, hsa-miR-372, hsa-miR-373, hsa-miR-376c, hsa-miR-378*, hsa-miR-410, hsa-miR-421, hsa-miR-431, hsa-miR-483-5p, hsa-miR-485-3p, hsa-miR-498, hsa-miR-509-3-5p, hsa-miR-509-5p, hsa-miR-512-3p, hsa-miR-513a-3p, hsa-miR-514, hsa-miR-516b, hsa-miR-519d, hsa-miR-520a-3p, hsa-miR-520b, hsa-miR-520c-3p, hsa-miR-520d-3p, hsa-miR-520d-5p, hsa-miR-520e, hsa-miR-520g, hsa-miR-520h, hsa-miR-524-5p, hsa-miR-526b*, hsa-miR-545, hsa-miR-548l, hsa-miR-548n, hsa-miR-548p, hsa-miR-552, hsa-miR-556-3p, hsa-miR-561, hsa-miR-590-3p, hsa-miR-607, hsa-miR-629, hsa-miR-630, hsa-miR-641, hsa-miR-643, hsa-miR-656, hsa-miR-671-5p, hsa-miR-744*, hsa-miR-892a, hsa-miR-924, hsa-miR-93, and hsa-miR-944.

S100 calcium binding protein A7—S100A7—NM_{—}002963—hsa-miR-1253, hsa-miR-185*, hsa-miR-30c-1*, hsa-miR-30c-2*, hsa-miR-516b, hsa-miR-518c*, hsa-miR-593, hsa-miR-641, and hsa-miR-92a-2*.

Hydroxyprostaglandin dehydrogenase 15—(NAD)—HPGD—NM_{—}000860—hsa-abi-13106, hsa-let-7c*, hsa-let-7d*, hsa-miR-1183, hsa-miR-1200, hsa-miR-1201, hsa-miR-1208, hsa-miR-1225-5p, hsa-miR-1252, hsa-miR-1255a, hsa-miR-1255b, hsa-miR-1259, hsa-miR-126*, hsa-miR-1270, hsa-miR-1284, hsa-miR-1285, hsa-miR-129-5p, hsa-miR-1297, hsa-miR-1308, hsa-miR-130a*, hsa-miR-1324, hsa-miR-133a, hsa-miR-133b, hsa-miR-137, hsa-miR-148a*, hsa-miR-154, hsa-miR-1826, hsa-miR-186*, hsa-miR-190, hsa-miR-190b, hsa-miR-203, hsa-miR-20b*, hsa-miR-21, hsa-miR-216a, hsa-miR-218, hsa-miR-223*, hsa-miR-23a, hsa-miR-23b, hsa-miR-26a, hsa-miR-26a-1*, hsa-miR-26a-2*, hsa-miR-26b, hsa-miR-297, hsa-miR-29a*, hsa-miR-29b-1*, hsa-miR-30b*, hsa-miR-31, hsa-miR-32*, hsa-miR-324-3p, hsa-miR-330-3p, hsa-miR-335*, hsa-miR-361-5p, hsa-miR-374a, hsa-miR-374b, hsa-miR-374b*, hsa-miR-376c, hsa-miR-379*, hsa-miR-383, hsa-miR-410, hsa-miR-411*, hsa-miR-421, hsa-miR-424*, hsa-miR-452*, hsa-miR-485-5p, hsa-miR-488, hsa-miR-488*, hsa-miR-498, hsa-miR-499-3p, hsa-miR-511, hsa-miR-516b, hsa-miR-518a-5p, hsa-miR-519a, hsa-miR-519b-3p, hsa-miR-519c-3p, hsa-miR-527, hsa-miR-543, hsa-miR-548a-5p, hsa-miR-548b-5p, hsa-miR-548c-5p, hsa-miR-548d-5p, hsa-miR-548g, hsa-miR-548h, hsa-miR-548i, hsa-miR-548j, hsa-miR-548l, hsa-miR-548p, hsa-miR-554, hsa-miR-559, hsa-miR-561, hsa-miR-562, hsa-miR-578, hsa-miR-579, hsa-miR-582-5p, hsa-miR-587, hsa-miR-589*, hsa-miR-590-5p, hsa-miR-607, hsa-miR-612, hsa-miR-616, hsa-miR-620, hsa-miR-633, hsa-miR-643, hsa-miR-654-3p, hsa-miR-655, hsa-miR-770-5p, hsa-miR-9, hsa-miR-935, and hsa-miR-944.

CEACAM7—CEACAM7—NM_{—}006890—hsa-let-7c*, hsa-miR-103, hsa-miR-107, hsa-miR-1205, hsa-miR-1236, hsa-miR-1254, hsa-miR-125a-3p, hsa-miR-1261, hsa-miR-1268, hsa-miR-1272, hsa-miR-1274a, hsa-miR-1274b, hsa-miR-1285, hsa-miR-129-5p, hsa-miR-1300, hsa-miR-1302, hsa-miR-1303, hsa-miR-1324, hsa-miR-136, hsa-miR-149, hsa-miR-154, hsa-miR-154*, hsa-miR-15a, hsa-miR-15b, hsa-miR-16, hsa-miR-182, hsa-miR-1827, hsa-miR-186, hsa-miR-188-5p, hsa-miR-194*, hsa-miR-195, hsa-miR-200a*, hsa-miR-200b*, hsa-miR-21, hsa-miR-223, hsa-miR-26b*, hsa-miR-28-5p, hsa-miR-30a*, hsa-miR-30d*, hsa-miR-30e*, hsa-miR-324-5p, hsa-miR-345, hsa-miR-378, hsa-miR-422a, hsa-miR-424, hsa-miR-425, hsa-miR-450b-3p, hsa-miR-450b-5p, hsa-miR-455-3p, hsa-miR-485-3p, hsa-miR-487a, hsa-miR-497, hsa-miR-509-3p, hsa-miR-516a-3p, hsa-miR-516b, hsa-miR-516b*, hsa-miR-517b, hsa-miR-518d-5p, hsa-miR-518e*, hsa-miR-518f*, hsa-miR-519a*, hsa-miR-519b-5p, hsa-miR-519c-5p, hsa-miR-520c-5p, hsa-miR-522*, hsa-miR-523*, hsa-miR-524-3p, hsa-miR-525-3p, hsa-miR-526a, hsa-miR-544, hsa-miR-548a-3p, hsa-miR-548e, hsa-miR-548f, hsa-miR-548g, hsa-miR-552, hsa-miR-561, hsa-miR-578, hsa-miR-583, hsa-miR-590-5p, hsa-miR-612, hsa-miR-617, hsa-miR-618, hsa-miR-622, hsa-miR-627, hsa-miR-628-5p, hsa-miR-640, hsa-miR-647, hsa-miR-653, hsa-miR-660, hsa-miR-663b, hsa-miR-708, hsa-miR-758, hsa-miR-766, hsa-miR-769-3p, hsa-miR-873, hsa-miR-892b, hsa-miR-935, hsa-miR-936, and hsa-miR-940.

Trefoil factor 1—TFF1—NM_{—}003225—hsa-miR-187, hsa-miR-298, hsa-miR-326, hsa-miR-330-5p, hsa-miR-889, and hsa-miR-92b*.

Prolactin-induced protein—PIP—NM_{—}002652—hsa-miR-1324, hsa-miR-140-3p, hsa-miR-644, hsa-miR-877, and hsa-miR-922.

Secretagogin—SCGN—NM_{—}006998—hsa-miR-22 hsa-miR-920, hsa-miR-330-3p, hsa-miR-134, hsa-miR-1252, hsa-miR-548p, hsa-miR-329, hsa-miR-494, hsa-miR-30a*, hsa-miR-622, hsa-miR-324-3p, hsa-miR-220c, hsa-miR-939, hsa-miR-26b*, hsa-miR-653, hsa-miR-30d*, hsa-miR-30e*, hsa-miR-570, hsa-miR-1274a, hsa-miR-362-3p, hsa-miR-634, hsa-miR-1185, hsa-miR-30b*, hsa-miR-339-5p, hsa-miR-1228, hsa-miR-548c-3p, hsa-miR-1274b, hsa-miR-924, hsa-miR-892a, hsa-miR-627, hsa-miR-512-5p.

SWAP70—SWAP70—NM_{—}015055—hsa-miR-557, hsa-miR-200b, hsa-miR-520b, hsa-miR-34c-5p, hsa-miR-645, hsa-miR-16, hsa-miR-448, hsa-miR-515-5p, hsa-miR-380*, hsa-miR-22, hsa-miR-562, hsa-miR-1276, hsa-miR-125b-2*, hsa-miR-520g, hsa-miR-519b-3p, hsa-miR-513b, hsa-let-7g*, hsa-miR-563 7,hsa-miR-532-3p, hsa-miR-548a-5p, hsa-miR-559, hsa-miR-1267, hsa-miR-548b-5p, hsa-miR-516a-3p, hsa-miR-452, hsa-miR-320c, hsa-miR-520a-3p, hsa-miR-511, hsa-miR-513a-3p, hsa-miR-544, hsa-miR-379*, hsa-miR-335*, hsa-miR-507, hsa-miR-449a, hsa-miR-183*, hsa-miR-330-3p, hsa-miR-519c-3p, hsa-miR-129-5p, hsa-miR-943, hsa-miR-548n, hsa-miR-302e, hsa-let-7c*, hsa-miR-548c-5p, hsa-miR-574-3p, hsa-miR-134, hsa-miR-519a, hsa-miR-1303, hsa-miR-216b, hsa-miR-1252, hsa-miR-651, hsa-miR-548p, hsa-miR-586, hsa-miR-338-5p, hsa-miR-30b, hsa-miR-143*, hsa-miR-34a*, hsa-miR-374a*, hsa-miR-23a, hsa-miR-449b, hsa-miR-33a*, hsa-miR-302c, hsa-miR-302f, hsa-miR-548i, hsa-miR-516b*, hsa-miR-646, hsa-let-7b*, hsa-miR-1281, hsa-miR-494, hsa-miR-371-5p, hsa-miR-30a, hsa-miR-489, hsa-miR-1227, hsa-miR-373, hsa-miR-200c, hsa-miR-145, hsa-miR-552, hsa-miR-1286, hsa-miR-720, hsa-miR-302d, hsa-miR-1202, hsa-miR-15b, hsa-miR-320d, hsa-miR-590-3p, hsa-miR-495, hsa-miR-29c, hsa-miR-591, hsa-miR-1208, hsa-miR-34a, hsa-miR-29b, hsa-miR-579, hsa-miR-411*, hsa-miR-1259, hsa-miR-186*, hsa-miR-520c-3p, hsa-miR-302a, hsa-miR-888, hsa-miR-429, hsa-miR-29a, hsa-miR-570, hsa-miR-124, hsa-miR-517*, hsa-miR-548d-5p, hsa-miR-1294, hsa-miR-34b*, hsa-miR-148b*, hsa-miR-125a-3p, hsa-miR-1285, hsa-miR-512-3p, hsa-miR-520d-3p, hsa-miR-195, hsa-miR-450b-5p, hsa-miR-610, hsa-miR-519d, hsa-miR-545, hsa-miR-612, hsa-miR-9*, hsa-miR-548j, hsa-miR-607, hsa-miR-520e, hsa-miR-30d, hsa-miR-93, hsa-miR-29b-2*, hsa-miR-548b-3p, hsa-miR-942, hsa-miR-23b, hsa-miR-1274a, hsa-miR-567, hsa-miR-302d*, hsa-miR-328, hsa-miR-526b*, hsa-miR-548h, hsa-miR-744*, hsa-miR-519e*, hsa-let-7f-1*, hsa-miR-432*, hsa-miR-150, hsa-miR-30c, hsa-miR-181a-2*, hsa-miR-451, hsa-miR-221*, hsa-miR-20b, hsa-miR-378*, hsa-miR-640, hsa-miR-100*, hsa-miR-1224-3p, hsa-miR-106a, hsa-miR-138-2*, hsa-miR-18a*, hsa-miR-130a*, hsa-miR-520h, hsa-miR-320a, hsa-miR-197, hsa-miR-1299, hsa-miR-548c-3p, hsa-miR-17, hsa-miR-182, hsa-miR-320b, hsa-miR-297, hsa-miR-1274b, hsa-miR-424, hsa-miR-506, hsa-miR-302b, hsa-miR-626, hsa-miR-655, hsa-miR-924, hsa-miR-577, hsa-miR-15a, hsa-miR-632, hsa-miR-20a, hsa-miR-409-3p, hsa-miR-455-3p, hsa-let-7a*, hsa-miR-340, hsa-miR-140-3p, hsa-miR-106b, hsa-miR-497, hsa-miR-369-3p, hsa-miR-639, hsa-miR-302b*, hsa-miR-372, hsa-miR-367*, hsa-miR-497*, hsa-miR-433, hsa-miR-499-3p, hsa-miR-662, hsa-miR-1225-3p, hsa-miR-512-5p, hsa-miR-30e.

Cyclin Y—CCYN (C10orf9)—Variant 1 and Variant 2—NM_{—}145012 and NM_{—}181698—hsa-miR-194*, hsa-miR-520b, hsa-miR-16, hsa-miR-141, * hsa-miR-922, hsa-miR-493*, hsa-miR-130b*, hsa-let-7d, hsa-miR-875-5p, hsa-miR-513a-5p, hsa-miR-192, hsa-miR-550*, hsa-miR-222*, hsa-miR-551b*, hsa-miR-644, hsa-miR-204, hsa-let-7g*, hsa-miR-638, hsa-miR-890, hsa-miR-548a-5p, hsa-miR-559, hsa-miR-27a, hsa-miR-548b-5p, hsa-miR-516a-3p, hsa-miR-520a-3p, hsa-miR-511, hsa-miR-31*, hsa-miR-513a-3p, hsa-miR-148a*, hsa-miR-29b-1*, hsa-miR-330-3p, hsa-miR-561, hsa-miR-548n, hsa-miR-302e, hsa-miR-548c-5p, hsa-miR-603, hsa-miR-342-3p, hsa-miR-298, hsa-miR-508-5p, hsa-miR-130b, hsa-let-7e, hsa-miR-370, hsa-miR-1207-5p, hsa-miR-216b, hsa-miR-1252, hsa-miR-1246, hsa-miR-600, hsa-miR-586, hsa-miR-876-5p, hsa-miR-524-5p, hsa-miR-30b, hsa-miR-374a*, hsa-miR-301b, hsa-miR-337-3p, hsa-miR-33a*, hsa-miR-302c, hsa-miR-491-5p, hsa-miR-548i, hsa-miR-202, hsa-miR-520a-5p, hsa-miR-548d-3p, hsa-miR-516b*, hsa-let-7b*, hsa-miR-525-5p, hsa-miR-494, hsa-miR-371-5p, hsa-miR-30a, hsa-miR-29a*, hsa-miR-373, hsa-miR-130a, hsa-miR-220c, hsa-miR-26b, hsa-let-7f, hsa-miR-302d, hsa-miR-15b, hsa-miR-96*, hsa-miR-617, hsa-miR-599, hsa-miR-590-3p, hsa-miR-495, hsa-miR-1297, hsa-miR-7-1*, hsa-miR-215, hsa-miR-1253, hsa-miR-193a-3p, hsa-miR-361-5p, hsa-miR-186*, hsa-miR-200c*, hsa-miR-520c-3p, hsa-miR-770-5p, hsa-miR-509-3p, hsa-miR-144, hsa-miR-653, hsa-miR-302a, hsa-miR-553, hsa-miR-802, hsa-miR-592, hsa-miR-570, hsa-miR-105, hsa-let-7g, hsa-miR-124, hsa-miR-940, hsa-miR-548d-5p, hsa-miR-432, hsa-miR-769-3p, hsa-miR-768-3p, hsa-miR-450b-3p, hsa-miR-512-3p, hsa-miR-520d-3p, hsa-miR-195, hsa-miR-331-5p, hsa-miR-27b, hsa-miR-548j, hsa-miR-607, hsa-miR-520e, hsa-miR-30d, hsa-miR-193b, hsa-let-7i, hsa-miR-942, hsa-miR-1263, hsa-miR-545*, hsa-miR-539, hsa-miR-33b, hsa-let-7b, hsa-miR-139-5p, hsa-miR-301a, hsa-let-7a, hsa-miR-98, hsa-miR-203, hsa-miR-548h, hsa-miR-211, hsa-let-7f-1*, hsa-miR-1300, hsa-miR-628-5p, hsa-miR-30c, hsa-miR-205, hsa-miR-1284, hsa-miR-659, hsa-miR-199a-3p, hsa-miR-191*, hsa-miR-1225-5p, hsa-let-7c, hsa-miR-182, hsa-miR-454, hsa-miR-1264, hsa-miR-297, hsa-miR-548l, hsa-miR-424, hsa-miR-506, hsa-miR-302b, hsa-miR-655, hsa-miR-199b-3p, hsa-miR-137, hsa-miR-1278, hsa-miR-7-2*, hsa-miR-33a, hsa-miR-15a, hsa-miR-1244, hsa-miR-627, hsa-let-7a*, hsa-miR-497, hsa-let-7f-2*, hsa-miR-1197, hsa-miR-16-1*, hsa-miR-649, hsa-miR-372, hsa-miR-367*, hsa-miR-26a, hsa-miR-520d-5p, hsa-miR-30e.

Cell division cycle 123 homolog—CDC123 (C10orf7)—NM_{—}006023—hsa-miR-376b, hsa-miR-493*, hsa-miR-562, hsa-miR-92a-2*, hsa-miR-574-3p, hsa-miR-663, hsa-miR-1203, hsa-miR-374a*, hsa-miR-646, hsa-miR-376a, hsa-miR-145, hsa-miR-589*, hsa-miR-361-5p, hsa-miR-144, hsa-miR-886-5p, hsa-miR-125a-3p, hsa-miR-935, hsa-miR-101, hsa-miR-1257, hsa-miR-877*, hsa-miR-202*, hsa-miR-744, hsa-miR-584.

Alkb, alkylation repair homolog 1—ALKBH1—NM_{—}006020—hsa-miR-515-5p, hsa-miR-22, hsa-miR-922, hsa-miR-130b*, hsa-miR-875-5p, hsa-miR-1276, hsa-miR-520g, hsa-miR-374b, hsa-miR-551b*, hsa-miR-195*, hsa-miR-204, hsa-miR-96, hsa-miR-548a-5p, hsa-miR-490-3p, hsa-miR-559, hsa-miR-548b-5p, hsa-miR-16-2*, hsa-miR-575, hsa-miR-31, hsa-miR-379*, hsa-miR-486-3p, hsa-miR-335*, hsa-miR-541*, hsa-miR-548c-5p, hsa-miR-938, hsa-miR-134, hsa-miR-1231, hsa-miR-619, hsa-miR-651, hsa-miR-129*, hsa-miR-299-5p, hsa-miR-586, hsa-miR-186, hsa-miR-548i, hsa-miR-202, hsa-miR-520a-5p, hsa-miR-548d-3p, hsa-miR-525-5p, hsa-miR-145, hsa-miR-1286, hsa-miR-664, hsa-miR-1269, hsa-miR-96*, hsa-miR-590-3p, hsa-miR-1271, hsa-miR-591, hsa-miR-1253, hsa-miR-411*, hsa-miR-186*, hsa-miR-1233, hsa-miR-144, hsa-miR-802, hsa-miR-570, hsa-miR-548d-5p, hsa-miR-769-3p, hsa-miR-593, hsa-miR-450b-3p, hsa-miR-198, hsa-miR-545, hsa-miR-548j, hsa-miR-323-3p, hsa-miR-607, hsa-miR-1258, hsa-miR-629, hsa-miR-942, hsa-miR-1322, hsa-miR-23a*, hsa-miR-1274a, hsa-miR-539, hsa-miR-23b*, hsa-miR-33b, hsa-miR-149*, hsa-miR-139-5p, hsa-miR-22*, hsa-miR-548h, hsa-miR-744*, hsa-miR-211, hsa-miR-519e*, hsa-miR-1300, hsa-miR-1321, hsa-miR-550, hsa-miR-520h, hsa-miR-339-5p, hsa-miR-129-3p, hsa-miR-196a*, hsa-miR-485-5p, hsa-miR-488*, hsa-miR-577, hsa-miR-33a, hsa-miR-188-5p, hsa-miR-374a, hsa-miR-556-3p, hsa-miR-1272, hsa-miR-340, hsa-miR-1265, hsa-miR-135b*.

Transducer of ERBB2—TOB2—NM_{—}016272—hsa-miR-194*, hsa-miR-509-3-5p, hsa-miR-877, hsa-miR-520b, hsa-miR-1266, hsa-miR-34c-5p, hsa-miR-32, hsa-miR-642, hsa-miR-16, hsa-miR-500, hsa-miR-20b*, hsa-miR-542-3p, hsa-miR-493*, hsa-miR-138, hsa-miR-130b*, hsa-let-7d, hsa-miR-875-5p, hsa-miR-513a-5p, hsa-miR-766, hsa-miR-1184, hsa-miR-9, hsa-miR-615-5p, hsa-miR-1301, hsa-miR-125a-5p, hsa-miR-483-3p, hsa-miR-548g, hsa-let-7g*, hsa-miR-1826, hsa-miR-335, hsa-miR-92a-2*, hsa-miR-520a-3p, hsa-miR-661, hsa-miR-31, hsa-miR-544, hsa-miR-143, hsa-miR-24-1*, hsa-miR-449a, hsa-miR-378, hsa-miR-1237, hsa-miR-561, hsa-miR-217, hsa-miR-1288, hsa-miR-302e, hsa-miR-93*, hsa-miR-603, hsa-miR-508-5p, hsa-miR-92b, hsa-let-7e, hsa-miR-370, hsa-miR-1207-5p, hsa-miR-1287, hsa-miR-623, hsa-miR-216b, hsa-miR-103, hsa-miR-630, hsa-miR-548p, hsa-miR-367, hsa-miR-543, hsa-miR-25, hsa-miR-365, hsa-miR-449b, hsa-miR-618, hsa-miR-302c, hsa-miR-491-5p, hsa-miR-24-2*, hsa-miR-646, hsa-miR-329, hsa-miR-622, hsa-miR-324-3p, hsa-miR-548o, hsa-miR-373, hsa-miR-874, hsa-miR-548f, hsa-miR-569, hsa-miR-484, hsa-let-7f, hsa-miR-302d, hsa-miR-765, hsa-miR-15b, hsa-miR-635, hsa-miR-1253, hsa-miR-34a, hsa-miR-135a, hsa-miR-605, hsa-miR-768-5p, hsa-miR-186*, hsa-miR-147, hsa-miR-520c-3p, hsa-miR-518a-5p, hsa-miR-608, hsa-miR-185*, hsa-miR-1262, hsa-miR-1323, hsa-miR-125b, hsa-miR-302a, hsa-miR-488, hsa-miR-105, hsa-miR-508-3p, hsa-let-7g, hsa-miR-940, hsa-miR-34b*, hsa-miR-125a-3p, hsa-miR-512-3p, hsa-miR-520d-3p, hsa-miR-195, hsa-miR-450b-5p, hsa-miR-610, hsa-miR-92a-1*, hsa-miR-20a*, hsa-miR-545, hsa-miR-504, hsa-miR-135b, hsa-miR-520e, hsa-miR-192*, hsa-miR-1206, hsa-miR-1260, hsa-let-7i, hsa-miR-422a, hsa-miR-1274a, hsa-miR-362-3p, hsa-miR-548a-3p, hsa-miR-1255a, hsa-miR-33b, hsa-let-7b, hsa-miR-149*, hsa-miR-330-5p, hsa-miR-551a, hsa-let-7a, hsa-miR-98, hsa-miR-571, hsa-miR-92a, hsa-miR-708*, hsa-miR-597, hsa-miR-1185, hsa-miR-361-3p, hsa-miR-432*, hsa-miR-1300, hsa-miR-583, hsa-miR-501-5p, hsa-miR-342-5p, hsa-miR-221*, hsa-miR-527, hsa-miR-17*, hsa-miR-556-5p, hsa-miR-1321, hsa-miR-640, hsa-miR-631, hsa-miR-18a*, hsa-miR-30b*, hsa-miR-550, hsa-let-7c, hsa-miR-202*, hsa-miR-1827, hsa-miR-24, hsa-miR-1299, hsa-miR-548c-3p, hsa-miR-663b, hsa-miR-656, hsa-miR-1274b, hsa-miR-424, hsa-miR-654-5p, hsa-miR-302b, hsa-miR-548e, hsa-miR-655, hsa-miR-485-5p, hsa-miR-122, hsa-miR-33a, hsa-miR-377*, hsa-miR-107, hsa-miR-15a, hsa-miR-126*, hsa-miR-363, hsa-miR-1272, hsa-miR-509-5p, hsa-miR-944, hsa-miR-574-5p, hsa-miR-497, hsa-miR-296-3p, hsa-miR-1197, hsa-miR-551b, hsa-miR-372, hsa-miR-326, hsa-miR-602, hsa-miR-1255b, hsa-miR-541, hsa-miR-1205, hsa-miR-671-5p.

Xenotropic and polytropic retrovirus receptor—XPR1—NM_{—}004736—hsa-miR-140-5p, hsa-miR-637, hsa-miR-520b, hsa-miR-32, hsa-miR-16, hsa-miR-22, hsa-miR-216a, hsa-miR-562, hsa-miR-548g, hsa-miR-1289, hsa-miR-30c-1*, hsa-miR-548a-5p, hsa-miR-1826, hsa-miR-647, hsa-miR-559, hsa-miR-1248, hsa-miR-548b-5p, hsa-miR-520a-3p, hsa-miR-142-5p, hsa-miR-335*, hsa-miR-548m, hsa-miR-378, hsa-miR-1226*, hsa-miR-194, hsa-miR-541*, hsa-miR-548n, hsa-miR-302e, hsa-miR-548c-5p, hsa-miR-1825, hsa-miR-128, hsa-miR-92b, hsa-miR-199a-5p, hsa-miR-875-3p, hsa-miR-1252, hsa-miR-367, hsa-miR-30b, hsa-miR-25, hsa-miR-374a*, hsa-miR-30c-2*, hsa-miR-1236, hsa-miR-1305, hsa-miR-33a*, hsa-miR-302c, hsa-miR-548i, hsa-miR-202, hsa-miR-606, hsa-miR-548d-3p, hsa-miR-146a*, hsa-miR-145*, hsa-miR-582-3p, hsa-miR-30a, hsa-miR-373, hsa-miR-616*, hsa-miR-373*, hsa-miR-548f, hsa-miR-26b, hsa-miR-573, hsa-miR-302d, hsa-miR-628-3p, hsa-miR-15b, hsa-miR-590-3p, hsa-miR-502-5p, hsa-miR-513c, hsa-miR-1297, hsa-miR-7-1*, hsa-miR-26b*, hsa-miR-921, hsa-miR-579, hsa-miR-361-5p, hsa-miR-520c-3p, hsa-miR-633, hsa-miR-302a, hsa-miR-802, hsa-miR-624*, hsa-miR-570, hsa-miR-508-3p, hsa-miR-548d-5p, hsa-miR-512-3p, hsa-miR-520d-3p, hsa-miR-195, hsa-miR-331-5p, hsa-miR-299-3p, hsa-miR-664*, hsa-miR-548j, hsa-miR-520e, hsa-miR-200a, hsa-miR-30d, hsa-miR-1260, hsa-miR-422a, hsa-miR-539, hsa-miR-548a-3p, hsa-miR-33b*, hsa-miR-302d*, hsa-miR-141, hsa-miR-223, hsa-miR-889, hsa-miR-22*, hsa-miR-203, hsa-miR-548h, hsa-miR-92a, hsa-miR-30c, hsa-miR-583, hsa-miR-501-5p, hsa-miR-199b-5p, hsa-miR-205, hsa-miR-877*, hsa-miR-18a*, hsa-miR-519e, hsa-miR-548c-3p, hsa-miR-106a*, hsa-miR-382, hsa-miR-182, hsa-miR-424, hsa-miR-302b, hsa-miR-548e, hsa-miR-655, hsa-miR-888*, hsa-miR-892a, hsa-miR-122, hsa-miR-7-2*, hsa-miR-377*, hsa-miR-15a, hsa-miR-126*, hsa-miR-363, hsa-miR-515-3p, hsa-miR-497, hsa-miR-302b*, hsa-miR-372, hsa-miR-223*, hsa-miR-595, hsa-miR-26a, hsa-miR-891b, hsa-miR-671-5p, hsa-miR-584, hsa-miR-30e.

CD59 molecule—CD59—Variant 1, Variant 2, Variant 3, Variant 4, Variant 5, Variant 6, Variant 7 and Variant 8—NM_{—}203330, NM_{—}000602, NM_{—}203329, NM_{—}203331, NM_{—}001127223, NM_{—}001127225, NM_{—}001127226 and NM_{—}001127227—hsa-miR-194*, hsa-miR-140-5p, hsa-miR-124*, hsa-miR-665, hsa-miR-518d-5p, hsa-miR-510, hsa-miR-200b, hsa-miR-509-3-5p, hsa-miR-877, hsa-miR-490-5p, hsa-miR-1266, hsa-miR-32, hsa-miR-1254, hsa-miR-642, hsa-miR-645, hsa-miR-380, hsa-miR-448, hsa-miR-519a*, hsa-miR-27b*, hsa-miR-141*, hsa-miR-522*, hsa-miR-22, hsa-miR-922, hsa-miR-648, hsa-miR-216a, hsa-miR-562, hsa-let-7d, hsa-miR-513a-5p, hsa-miR-766, hsa-miR-650, hsa-miR-9, hsa-miR-133a, hsa-miR-125b-2*, hsa-miR-616, hsa-miR-615-5p, hsa-miR-181a, hsa-miR-195*, hsa-miR-644, hsa-miR-548g, hsa-miR-204, hsa-miR-96, hsa-miR-1179, hsa-miR-214, hsa-miR-30c-1*, hsa-miR-549, hsa-miR-548a-5p, hsa-miR-376c, hsa-miR-1826, hsa-miR-647, hsa-miR-559, hsa-miR-1248, hsa-miR-335, hsa-miR-920, hsa-miR-138-1*, hsa-miR-92a-2*, hsa-miR-363*, hsa-miR-548b-5p, hsa-miR-516a-3p, hsa-miR-16-2*, hsa-miR-575, hsa-miR-320c, hsa-miR-511, hsa-miR-544, hsa-miR-26a-1*, hsa-miR-24-1*, hsa-miR-486-3p, hsa-miR-335*, hsa-miR-29b-1*, hsa-miR-548m, hsa-miR-183*, hsa-miR-378, hsa-miR-330-3p, hsa-miR-28-5p, hsa-miR-1226*, hsa-miR-190, hsa-miR-561, hsa-miR-129-5p, hsa-miR-217, hsa-miR-892b, hsa-miR-885-5p, hsa-miR-93*, hsa-miR-548c-5p, hsa-miR-603, hsa-miR-342-3p, hsa-miR-508-5p, hsa-miR-92b, hsa-let-7e, hsa-miR-370, hsa-miR-199a-5p, hsa-miR-1287, hsa-miR-623, hsa-miR-1252, hsa-miR-580, hsa-miR-660, hsa-miR-1246, hsa-miR-379, hsa-miR-600, hsa-miR-586, hsa-miR-367, hsa-miR-582-5p, hsa-miR-524-5p, hsa-miR-25, hsa-miR-516a-5p, hsa-miR-23a, hsa-miR-365, hsa-miR-30c-2*, hsa-miR-1236, hsa-miR-618, hsa-miR-1305, hsa-miR-548i, hsa-miR-520a-5p, hsa-miR-24-2*, hsa-miR-641, hsa-miR-212, hsa-miR-548d-3p, hsa-miR-516b*, hsa-miR-329, hsa-let-7b*, hsa-miR-708, hsa-miR-146a*, hsa-miR-153, hsa-miR-145*, hsa-miR-525-5p, hsa-miR-411, hsa-miR-30a*, hsa-miR-371-5p, hsa-miR-29a*, hsa-miR-324-3p, hsa-miR-548o, hsa-miR-1227, hsa-miR-200c, hsa-miR-581, hsa-miR-874, hsa-miR-548f, hsa-miR-220c, hsa-miR-664, hsa-miR-484, hsa-let-7f, hsa-miR-765, hsa-miR-320d, hsa-miR-599, hsa-miR-590-3p, hsa-miR-502-5p, hsa-miR-1271, hsa-miR-29c, hsa-miR-513c, hsa-miR-132, hsa-miR-7-1*, hsa-miR-589, hsa-miR-635, hsa-miR-767-3p, hsa-miR-589*, hsa-miR-190b, hsa-miR-1200, hsa-miR-29b, hsa-miR-135a, hsa-miR-579, hsa-miR-26a-2*, hsa-miR-146a, hsa-miR-768-5p, hsa-miR-1259, hsa-miR-374b*, hsa-miR-186*, hsa-miR-147, hsa-miR-873, hsa-miR-518a-5p, hsa-miR-27a*, hsa-miR-770-5p, hsa-miR-18b, hsa-miR-1226, hsa-miR-144, hsa-miR-1323, hsa-miR-323-5p, hsa-miR-220b, hsa-miR-505*, hsa-miR-1224-5p, hsa-miR-888, hsa-miR-30d*, hsa-miR-802, hsa-miR-568, hsa-miR-30e*, hsa-miR-21*, hsa-miR-429, hsa-miR-29a, hsa-miR-624*, hsa-miR-488, hsa-miR-105, hsa-let-7g, hsa-miR-124, hsa-miR-517*, hsa-miR-155, hsa-miR-548d-5p, hsa-miR-325, hsa-miR-1294, hsa-miR-588, hsa-miR-210, hsa-miR-593, hsa-miR-518f*, hsa-miR-768-3p, hsa-miR-148b*, hsa-miR-1285, hsa-miR-15a*, hsa-miR-610, hsa-miR-198, hsa-miR-20a*, hsa-miR-181d, hsa-miR-504, hsa-miR-935, hsa-miR-612, hsa-miR-135b, hsa-miR-664*, hsa-miR-548j, hsa-miR-323-3p, hsa-miR-607, hsa-miR-520c-5p, hsa-miR-200a, hsa-miR-578, hsa-miR-629*, hsa-miR-629, hsa-miR-29b-2*, hsa-miR-934, hsa-miR-518c*, hsa-miR-548b-3p, hsa-let-7i, hsa-miR-942, hsa-miR-422a, hsa-miR-1292, hsa-miR-23b, hsa-miR-1322, hsa-miR-23a*, hsa-miR-362-3p, hsa-miR-545*, hsa-miR-609, hsa-miR-548a-3p, hsa-miR-23b*, hsa-miR-33b, hsa-miR-146b-5p, hsa-miR-634, hsa-miR-516b, hsa-miR-526b, hsa-miR-133b, hsa-let-7b, hsa-miR-302d*, hsa-miR-330-5p, hsa-miR-139-5p, hsa-miR-141, hsa-miR-223, hsa-let-7a, hsa-miR-22*, hsa-miR-98, hsa-miR-203, hsa-miR-548h, hsa-miR-92a, hsa-miR-381, hsa-miR-211, hsa-miR-361-3p, hsa-miR-518e*, hsa-miR-593*, hsa-let-7f-1*, hsa-miR-432*, hsa-miR-1300, hsa-miR-628-5p, hsa-miR-181c, hsa-miR-526a, hsa-miR-760, hsa-miR-583, hsa-miR-501-5p, hsa-miR-199b-5p, hsa-miR-342-5p, hsa-miR-18a, hsa-miR-224, hsa-miR-221*, hsa-miR-527, hsa-miR-17*, hsa-miR-378*, hsa-miR-1256, hsa-miR-556-5p, hsa-miR-523*, hsa-miR-1224-3p, hsa-miR-877*, hsa-miR-149, hsa-miR-18a*, hsa-miR-130a*, hsa-miR-550, hsa-miR-320a, hsa-miR-1225-5p, hsa-let-7c, hsa-miR-197, hsa-miR-486-5p, hsa-miR-202*, hsa-miR-1228, hsa-miR-1827, hsa-miR-1299, hsa-miR-135a*, hsa-miR-196a*, hsa-miR-500*, hsa-miR-182, hsa-miR-320b, hsa-miR-1264, hsa-miR-297, hsa-miR-345, hsa-miR-654-5p, hsa-miR-506, hsa-miR-181b, hsa-miR-1283, hsa-miR-548e, hsa-miR-1251, hsa-miR-655, hsa-miR-485-5p, hsa-miR-888*, hsa-miR-924, hsa-miR-892a, hsa-miR-488*, hsa-miR-577, hsa-miR-122, hsa-miR-7-2*, hsa-miR-33a, hsa-miR-196a, hsa-miR-377*, hsa-miR-300, hsa-miR-425, hsa-miR-627, hsa-miR-423-5p, hsa-miR-196b, hsa-miR-363, hsa-miR-519b-5p, hsa-let-7a*, hsa-miR-338-3p, hsa-miR-509-5p, hsa-miR-574-5p, hsa-miR-1265, hsa-let-7f-2*, hsa-miR-1197, hsa-miR-519c-5p, hsa-miR-16-1*, hsa-miR-302b*, hsa-miR-649, hsa-miR-326, hsa-miR-1183, hsa-miR-602, hsa-miR-1234, hsa-miR-541, hsa-miR-520d-5p, hsa-miR-891b, hsa-miR-1225-3p, hsa-miR-671-5p.

Low density lipoprotein-related protein 2—LRP2—NM_{—}004525—hsa-miR-376b, hsa-miR-557, hsa-miR-510, hsa-miR-520b, hsa-miR-1254, hsa-miR-380, hsa-miR-16, hsa-miR-922, hsa-miR-875-5p, hsa-miR-222*, hsa-miR-454*, hsa-miR-195*, hsa-miR-204, hsa-let-7g*, hsa-miR-549, hsa-miR-218-1*, hsa-miR-1304, hsa-miR-548a-5p, hsa-miR-559, hsa-miR-335, hsa-miR-548b-5p, hsa-miR-16-2*, hsa-miR-520a-3p, hsa-miR-142-5p, hsa-miR-335*, hsa-miR-507, hsa-miR-378, hsa-miR-1237, hsa-miR-19b, hsa-miR-129-5p, hsa-miR-194, hsa-miR-302e, hsa-miR-548c-5p, hsa-miR-128, hsa-miR-298, hsa-miR-130b, hsa-miR-103, hsa-miR-148a, hsa-miR-576-5p, hsa-miR-876-5p, hsa-miR-338-5p, hsa-miR-34a*, hsa-miR-301b, hsa-miR-302c, hsa-miR-302f, hsa-miR-548i, hsa-miR-218-2*, hsa-miR-606, hsa-miR-548d-3p, hsa-let-7b*, hsa-miR-145*, hsa-miR-582-3p, hsa-miR-376a, hsa-miR-373, hsa-miR-130a, hsa-miR-145, hsa-miR-616*, hsa-miR-148b, hsa-miR-373*, hsa-miR-220c, hsa-miR-1286, hsa-miR-664, hsa-miR-1269, hsa-miR-302d, hsa-miR-15b, hsa-miR-590-3p, hsa-miR-7-1*, hsa-miR-921, hsa-miR-146a, hsa-miR-520c-3p, hsa-miR-518a-5p, hsa-miR-384, hsa-miR-302a, hsa-miR-888, hsa-miR-802, hsa-miR-548d-5p, hsa-miR-148b*, hsa-miR-125a-3p, hsa-miR-520d-3p, hsa-miR-195, hsa-miR-198, hsa-miR-452*, hsa-miR-548j, hsa-miR-520e, hsa-miR-578, hsa-miR-29b-2*, hsa-miR-518c*, hsa-miR-152, hsa-miR-422a, hsa-miR-101, hsa-miR-539, hsa-miR-146b-5p, hsa-miR-139-5p, hsa-miR-301a, hsa-miR-548h, hsa-miR-625*, hsa-miR-211, hsa-let-7f-1*, hsa-miR-1284, hsa-miR-527, hsa-miR-199a-3p, hsa-miR-877*, hsa-miR-30b*, hsa-miR-1299, hsa-miR-663b, hsa-miR-500*, hsa-miR-182, hsa-miR-454, hsa-miR-548k, hsa-miR-424, hsa-miR-302b, hsa-miR-485-5p, hsa-miR-199b-3p, hsa-miR-1278, hsa-miR-7-2*, hsa-miR-107, hsa-miR-19a, hsa-miR-15a, hsa-miR-556-3p, hsa-miR-409-3p, hsa-let-7a*, hsa-miR-944, hsa-miR-497, hsa-miR-1197, hsa-miR-372, hsa-miR-595, hsa-miR-499-3p, hsa-miR-744, hsa-miR-671-5p.

Proteolipid protein 2—PLP2—NM_{—}002668—hsa-miR-517a, hsa-miR-665, hsa-miR-1254, hsa-miR-642, hsa-miR-92a-2*, hsa-miR-220a, hsa-miR-378, hsa-miR-298, hsa-miR-1296, hsa-miR-33a*, hsa-miR-491-5p, hsa-miR-218-2*, hsa-miR-146a*, hsa-miR-582-3p, hsa-miR-1, hsa-miR-664, hsa-miR-765, hsa-miR-608, hsa-miR-220b, hsa-miR-124, hsa-miR-588, hsa-miR-422a, hsa-miR-539, hsa-miR-571, hsa-miR-150, hsa-miR-583, hsa-miR-151-5p, hsa-miR-758, hsa-miR-7, hsa-miR-506, hsa-miR-206, hsa-miR-409-3p, hsa-miR-296-3p, hsa-miR-517c, hsa-miR-613.

Mitogen-activated protein kinase 14—MAPK14—Variant 1 and Variant 2—NM_{—}001315 and NM_{—}139012—hsa-miR-124*, hsa-miR-637, hsa-miR-510, hsa-miR-1266, hsa-miR-642, hsa-miR-22, hsa-miR-513a-5p, hsa-miR-766, hsa-miR-133a, hsa-miR-520g, hsa-miR-615-5p, hsa-miR-550*, hsa-miR-125a-5p, hsa-miR-532-3p, hsa-miR-377, hsa-miR-548a-5p, hsa-miR-559, hsa-miR-27a, hsa-miR-1248, hsa-miR-335, hsa-miR-92a-2*, hsa-miR-548b-5p, hsa-miR-575, hsa-miR-143, hsa-miR-142-5p, hsa-miR-486-3p, hsa-miR-335*, hsa-miR-19b, hsa-miR-1178, hsa-miR-548c-5p, hsa-miR-603, hsa-miR-128, hsa-miR-587, hsa-miR-1303, hsa-miR-1280, hsa-miR-216b, hsa-miR-548p, hsa-miR-582-5p, hsa-miR-338-5p, hsa-miR-23a, hsa-miR-1236, hsa-miR-337-3p, hsa-miR-10a, hsa-miR-1201, hsa-miR-548i, hsa-miR-520a-5p, hsa-miR-92b*, hsa-miR-646, hsa-let-7b*, hsa-miR-145*, hsa-miR-525-5p, hsa-miR-489, hsa-miR-622, hsa-miR-1227, hsa-miR-145, hsa-miR-616*, hsa-miR-874, hsa-miR-373*, hsa-miR-1286, hsa-miR-573, hsa-miR-188-3p, hsa-miR-589, hsa-miR-589*, hsa-miR-1259, hsa-miR-200c*, hsa-miR-518a-5p, hsa-miR-185*, hsa-miR-125b, hsa-miR-488, hsa-miR-570, hsa-miR-124, hsa-miR-548d-5p, hsa-miR-769-3p, hsa-miR-450b-3p, hsa-miR-144*, hsa-miR-545, hsa-miR-27b, hsa-miR-548j, hsa-miR-607, hsa-miR-200a, hsa-miR-629*, hsa-miR-155*, hsa-miR-1206, hsa-miR-139-3p, hsa-miR-518c*, hsa-miR-548b-3p, hsa-miR-942, hsa-miR-23b, hsa-miR-539, hsa-miR-33b, hsa-miR-516b, hsa-miR-133b, hsa-miR-141, hsa-miR-889, hsa-miR-548h, hsa-miR-381, hsa-miR-1185, hsa-let-7f-1*, hsa-miR-432*, hsa-let-7e*, hsa-miR-224, hsa-miR-1284, hsa-miR-527, hsa-miR-17*, hsa-miR-18a*, hsa-miR-130a*, hsa-miR-520h, hsa-miR-10b, hsa-miR-555, hsa-miR-339-5p, hsa-miR-24, hsa-miR-196a*, hsa-miR-1279, hsa-miR-1264, hsa-miR-506, hsa-miR-485-5p, hsa-miR-33a, hsa-miR-19a, hsa-miR-300, hsa-miR-632, hsa-let-7a*, hsa-miR-340, hsa-miR-16-1*, hsa-miR-346, hsa-miR-433.

MAPK14—Variant 3—NM_{—}139013—hsa-miR-605, hsa-miR-607.

MAPK14—Variant 4—NM_{—}139014—hsa-miR-124*, hsa-miR-637, hsa-miR-510, hsa-miR-1266, hsa-miR-642, hsa-miR-22, hsa-miR-513 a-5p, hsa-miR-766, hsa-miR-133 a, hsa-miR-520g, hsa-miR-615-5p, hsa-miR-550*, hsa-miR-125a-5p, hsa-miR-532-3p, hsa-miR-377, hsa-miR-548a-5p, hsa-miR-559, hsa-miR-27a, hsa-miR-1248, hsa-miR-335, hsa-miR-92a-2*, hsa-miR-548b-5p, hsa-miR-575, hsa-miR-320c, hsa-miR-143, hsa-miR-142-5p, hsa-miR-486-3p, hsa-miR-335*, hsa-miR-19b, hsa-miR-1178, hsa-miR-548c-5p, hsa-miR-603, hsa-miR-128, hsa-miR-587, hsa-miR-1303, hsa-miR-1280, hsa-miR-216b, hsa-miR-548p, hsa-miR-582-5p, hsa-miR-338-5p, hsa-miR-23a, hsa-miR-1236, hsa-miR-337-3p, hsa-miR-10a, hsa-miR-1201, hsa-miR-548i, hsa-miR-520a-5p, hsa-miR-92b*, hsa-miR-646, hsa-let-7b*, hsa-miR-145*, hsa-miR-525-5p, hsa-miR-489, hsa-miR-622, hsa-miR-1227, hsa-miR-145, hsa-miR-616*, hsa-miR-874, hsa-miR-373*, hsa-miR-1286, hsa-miR-573, hsa-miR-320d, hsa-miR-188-3p, hsa-miR-589, hsa-miR-589*, hsa-miR-1259, hsa-miR-200c*, hsa-miR-518a-5p, hsa-miR-608, hsa-miR-185*, hsa-miR-125b, hsa-miR-488, hsa-miR-570, hsa-miR-124, hsa-miR-548d-5p, hsa-miR-769-3p, hsa-miR-450b-3p, hsa-miR-144*, hsa-miR-545, hsa-miR-27b, hsa-miR-548j, hsa-miR-607, hsa-miR-200a, hsa-miR-629*, hsa-miR-155*, hsa-miR-1206, hsa-miR-139-3p, hsa-miR-518c*, hsa-miR-548b-3p, hsa-miR-942, hsa-miR-23b, hsa-miR-539, hsa-miR-33b, hsa-miR-516b, hsa-miR-133b, hsa-miR-141, hsa-miR-889, hsa-miR-548h, hsa-miR-381, hsa-miR-1185, hsa-let-7f-1*, hsa-miR-432*, hsa-let-7e*, hsa-miR-224, hsa-miR-1284, hsa-miR-527, hsa-miR-17*, hsa-miR-18a*, hsa-miR-130a*, hsa-miR-520h, hsa-miR-320a, hsa-miR-10b, hsa-miR-555, hsa-miR-339-5p, hsa-miR-24, hsa-miR-196a*, hsa-miR-1279, hsa-miR-320b, hsa-miR-1264, hsa-miR-506, hsa-miR-485-5p, hsa-miR-33 a, hsa-miR-19a, hsa-miR-300, hsa-miR-632, hsa-let-7a*, hsa-miR-340, hsa-miR-16-1*, hsa-miR-346, hsa-miR-433, hsa-miR-1293.

Matrix metallopeptidase 7—MMP7—NM_{—}002423—hsa-miR-181a, hsa-miR-379*, hsa-miR-302f, hsa-miR-641, hsa-miR-548d-3p, hsa-let-7b*, hsa-miR-489, hsa-miR-411*, hsa-miR-520f, hsa-miR-181d, hsa-let-7f-1*, hsa-miR-181c, hsa-miR-297, hsa-miR-181b, hsa-let-7a*.

Matrix Gla protein—MGP—NM000900—hsa-miR-637, hsa-miR-509-3-5p, hsa-miR-148a, hsa-miR-30a*, hsa-miR-148b, hsa-miR-1202, hsa-miR-30d*, hsa-miR-30e*, hsa-miR-155, hsa-miR-152, hsa-miR-342-5p, hsa-miR-17*, hsa-miR-548l, hsa-miR-509-5p, hsa-let-7f-2*.

Myeloid leukemia factor 1—MLF1—NM_{—}022443—hsa-miR-181a, hsa-miR-551b*, hsa-miR-885-5p, hsa-miR-338-5p, hsa-miR-543, hsa-miR-29a*, hsa-miR-548o, hsa-miR-590-3p, hsa-miR-29c, hsa-miR-7-1*, hsa-miR-29b, hsa-miR-361-5p, hsa-miR-509-3p, hsa-miR-1323, hsa-miR-29a, hsa-miR-105, hsa-miR-144*, hsa-miR-181d, hsa-miR-9*, hsa-miR-181c, hsa-miR-583, hsa-miR-205, hsa-miR-181b, hsa-miR-7-2*, hsa-miR-556-3p, hsa-miR-767-5p, hsa-miR-340.

FilaminB, beta (actin binding protein 278)—FLNB—NM_{—}001457—hsa-miR-557, hsa-miR-510, hsa-miR-509-3-5p, hsa-miR-16, hsa-miR-515-5p, hsa-miR-141*, hsa-miR-1290, hsa-miR-195*, hsa-miR-548g, hsa-let-7g*, hsa-miR-890, hsa-miR-647, hsa-miR-452, hsa-miR-16-2*, hsa-miR-575, hsa-miR-544, hsa-miR-507, hsa-miR-29b-1*, hsa-miR-183*, hsa-miR-1226*, hsa-miR-886-3p, hsa-miR-548p, hsa-miR-876-5p, hsa-miR-33a*, hsa-miR-92b*, hsa-miR-646, hsa-miR-145, hsa-miR-548f, hsa-miR-484, hsa-miR-296-5p, hsa-miR-15b, hsa-miR-1208, hsa-miR-568, hsa-miR-940, hsa-miR-588, hsa-miR-184, hsa-miR-148b*, hsa-miR-195, hsa-miR-545, hsa-miR-299-3p, hsa-miR-192*, hsa-miR-942, hsa-miR-609, hsa-miR-548a-3p, hsa-miR-1255a, hsa-miR-634, hsa-miR-302d*, hsa-miR-203, hsa-miR-519e*, hsa-miR-1300, hsa-miR-17*, hsa-miR-100*, hsa-miR-1245, hsa-miR-18a*, hsa-miR-24, hsa-miR-500*, hsa-miR-182, hsa-miR-424, hsa-miR-654-5p, hsa-miR-548e, hsa-miR-488*, hsa-miR-122, hsa-miR-15a, hsa-miR-632, hsa-miR-509-5p, hsa-miR-499-5p, hsa-miR-193b*, hsa-miR-497, hsa-miR-346, hsa-miR-302b*, hsa-miR-1255b, hsa-miR-541, hsa-miR-1205, hsa-miR-135b*.

Sodium channel modifier 1—SCNM1—NM_{—}024041—hsa-miR-148a*, hsa-miR-660, hsa-miR-1257.

Cornichon homolog 4—CNIH4 also known as HSPC163—NM_{—}014184—hsa-miR-20b*, hsa-miR-766, hsa-miR-182*, hsa-miR-548p, hsa-miR-593, hsa-miR-545, hsa-miR-149, hsa-miR-1183, hsa-miR-142-3p.

Receptor accessory protein 5—REEP5—NM_{—}005669—hsa-miR-200b, hsa-miR-877, hsa-miR-520b, hsa-miR-380, hsa-miR-448, hsa-miR-493*, hsa-miR-766, hsa-miR-550*, hsa-miR-1301, hsa-miR-181a, hsa-miR-551b*, hsa-miR-302c*, hsa-miR-548g, hsa-let-7g*, hsa-miR-218-1*, hsa-miR-548a-5p, hsa-miR-559, hsa-miR-548b-5p, hsa-miR-452, hsa-miR-520a-3p, hsa-miR-513a-3p, hsa-miR-379*, hsa-miR-335*, hsa-miR-29b-1*, hsa-miR-1226*, hsa-miR-302a*, hsa-miR-561, hsa-miR-541*, hsa-miR-548n, hsa-miR-217, hsa-miR-892b, hsa-miR-302e, hsa-let-7c*, hsa-miR-548c-5p, hsa-miR-587, hsa-miR-370, hsa-miR-1287, hsa-miR-424*, hsa-miR-524-5p, hsa-miR-186, hsa-miR-23a, hsa-miR-618, hsa-miR-33a*, hsa-miR-302c, hsa-miR-302f, hsa-miR-548i, hsa-miR-218-2*, hsa-miR-410, hsa-miR-421, hsa-miR-151-3p, hsa-miR-590-5p, hsa-miR-582-3p, hsa-miR-411, hsa-miR-29a*, hsa-miR-622, hsa-miR-373, hsa-miR-200c, hsa-miR-581, hsa-miR-616*, hsa-miR-373*, hsa-miR-548f, hsa-miR-484, hsa-miR-302d, hsa-miR-1202, hsa-miR-1324, hsa-miR-590-3p, hsa-miR-513c, hsa-miR-1208, hsa-miR-34c-3p, hsa-miR-767-3p, hsa-miR-26b*, hsa-miR-579, hsa-miR-411*, hsa-miR-146a, hsa-miR-200c*, hsa-miR-520c-3p, hsa-miR-1226, hsa-miR-144, hsa-miR-323-5p, hsa-miR-302a, hsa-miR-802, hsa-miR-429, hsa-miR-548d-5p, hsa-miR-768-3p, hsa-miR-34b*, hsa-miR-520d-3p, hsa-miR-610, hsa-miR-519d, hsa-miR-198, hsa-miR-20a*, hsa-miR-181d, hsa-miR-9*, hsa-miR-548j, hsa-miR-323-3p, hsa-miR-607, hsa-miR-520e, hsa-miR-629*, hsa-miR-93, hsa-miR-29b-2*, hsa-miR-554, hsa-miR-200b*, hsa-miR-23b, hsa-miR-1322, hsa-miR-545*, hsa-miR-548a-3p, hsa-miR-1255a, hsa-miR-146b-5p, hsa-miR-634, hsa-miR-136, hsa-miR-889, hsa-miR-526b*, hsa-miR-548h, hsa-miR-150, hsa-miR-181c, hsa-miR-20b, hsa-miR-640, hsa-miR-106a, hsa-miR-130a*, hsa-miR-522, hsa-miR-197, hsa-miR-17, hsa-miR-196a*, hsa-miR-656, hsa-miR-548k, hsa-miR-548l, hsa-miR-302b, hsa-miR-181b, hsa-miR-1283, hsa-miR-548e, hsa-miR-1251, hsa-miR-21, hsa-miR-924, hsa-miR-577, hsa-miR-20a, hsa-miR-425, hsa-miR-126*, hsa-miR-340, hsa-miR-140-3p, hsa-miR-106b, hsa-miR-16-1*, hsa-miR-372, hsa-miR-1255b, hsa-miR-520d-5p, hsa-miR-200a*, hsa-miR-1205, hsa-miR-671-5p.

Solute carrier family 25, member 33 (MGC4399)—SLC25A33—NM_{—}032315—hsa-miR-518d-5p, hsa-miR-519a*, hsa-miR-1180, hsa-miR-522*, hsa-miR-30a*, hsa-miR-371-5p, hsa-miR-552, hsa-miR-569, hsa-miR-32*, hsa-miR-30d*, hsa-miR-30e*, hsa-miR-517*, hsa-miR-518P, hsa-miR-125a-3p, hsa-miR-520c-5p, hsa-miR-518e*, hsa-miR-526a, hsa-miR-523*, hsa-miR-488*, hsa-miR-519b-5p, hsa-miR-519c-5p, hsa-miR-1234.

Solute carrier family 44, member 1—SLC44A1—NM_{—}080546—hsa-miR-16, hsa-miR-650, hsa-miR-101*, hsa-miR-551b*, hsa-miR-302c*, hsa-miR-337-5p, hsa-let-7g*, hsa-miR-28-3p, hsa-miR-1179, hsa-miR-548a-5p, hsa-miR-559, hsa-miR-1248, hsa-miR-548b-5p, hsa-miR-516a-3p, hsa-miR-452, hsa-miR-496, hsa-miR-513a-3p, hsa-miR-99a, hsa-miR-142-5p, hsa-miR-548m, hsa-miR-183*, hsa-miR-561, hsa-miR-548n, hsa-miR-885-5p, hsa-miR-548c-5p, hsa-miR-409-5p, hsa-miR-130b, hsa-miR-587, hsa-miR-199a-5p, hsa-miR-630, hsa-miR-548p, hsa-miR-586, hsa-miR-582-5p, hsa-miR-301b, hsa-miR-33a*, hsa-miR-302f, hsa-miR-548i, hsa-miR-641, hsa-miR-212, hsa-miR-548d-3p, hsa-miR-516b*, hsa-miR-329, hsa-let-7b*, hsa-miR-146a*, hsa-miR-29a*, hsa-miR-1, hsa-miR-130a, hsa-miR-548f, hsa-miR-147b, hsa-miR-720, hsa-miR-664, hsa-miR-32*, hsa-miR-15b, hsa-miR-617, hsa-miR-590-3p, hsa-miR-132, hsa-miR-589*, hsa-miR-643, hsa-miR-135a, hsa-miR-605, hsa-miR-768-5p, hsa-miR-147, hsa-miR-21*, hsa-miR-488, hsa-miR-570, hsa-miR-508-3p, hsa-miR-548d-5p, hsa-miR-148b*, hsa-miR-195, hsa-miR-135b, hsa-miR-548j, hsa-miR-323-3p, hsa-miR-607, hsa-miR-29b-2*, hsa-miR-192*, hsa-miR-518c*, hsa-miR-491-3p, hsa-miR-1322, hsa-miR-1274a, hsa-miR-362-3p, hsa-miR-545*, hsa-miR-548a-3p, hsa-miR-516b, hsa-miR-567, hsa-miR-301a, hsa-miR-571, hsa-miR-548h, hsa-miR-492, hsa-let-7f-1*, hsa-miR-181a-2*, hsa-miR-199b-5p, hsa-miR-556-5p, hsa-miR-99b, hsa-miR-548c-3p, hsa-miR-100, hsa-miR-1279, hsa-miR-454, hsa-miR-297, hsa-miR-1274b, hsa-miR-424, hsa-miR-1283, hsa-miR-548e, hsa-miR-206, hsa-miR-15a, hsa-miR-409-3p, hsa-miR-183, hsa-miR-455-3p, hsa-let-7a*, hsa-miR-338-3p, hsa-miR-944, hsa-miR-497, hsa-miR-369-3p, hsa-let-7f-2*, hsa-miR-613.

Transmembrane channel-like 4—TMC4—NM_{—}144686—hsa-miR-645, hsa-miR-192, hsa-miR-604, hsa-miR-320c, hsa-miR-661, hsa-miR-1268, hsa-miR-875-3p, hsa-miR-145, hsa-miR-320d, hsa-miR-215, hsa-miR-1291, hsa-miR-381, hsa-miR-758, hsa-miR-320a, hsa-miR-320b, hsa-miR-924, hsa-miR-488*, hsa-miR-300, hsa-miR-423-5p.

Zinc finger DHHC-type containing 2—ZDHHC2—NM_{—}016353—hsa-miR-509-3-5p, hsa-miR-490-5p, hsa-miR-32, hsa-miR-642, hsa-miR-380, hsa-miR-448, hsa-miR-515-5p, hsa-miR-27b*, hsa-miR-922, hsa-miR-192, hsa-miR-133a, hsa-miR-150*, hsa-miR-551b*, hsa-miR-302c*, hsa-miR-548g, hsa-miR-548a-5p, hsa-miR-559, hsa-miR-1248, hsa-miR-335, hsa-miR-127-5p, hsa-miR-548b-5p, hsa-miR-496, hsa-miR-513a-3p, hsa-miR-544, hsa-miR-143, hsa-miR-142-5p, hsa-miR-548m, hsa-miR-183*, hsa-miR-378, hsa-miR-561, hsa-miR-548n, hsa-miR-548c-5p, hsa-miR-603, hsa-miR-92b, hsa-miR-619, hsa-miR-587, hsa-miR-1287, hsa-miR-424*, hsa-miR-216b, hsa-miR-630, hsa-miR-576-5p, hsa-miR-367, hsa-miR-582-5p, hsa-miR-543, hsa-miR-25, hsa-miR-374a*, hsa-miR-186, hsa-miR-1236, hsa-miR-1305, hsa-miR-337-3p, hsa-miR-33a*, hsa-miR-302f, hsa-miR-548i, hsa-miR-606, hsa-miR-146a*, hsa-miR-590-5p, hsa-miR-145*, hsa-miR-582-3p, hsa-miR-371-5p, hsa-miR-622, hsa-miR-145, hsa-miR-616*, hsa-miR-373*, hsa-miR-548f, hsa-miR-590-3p, hsa-miR-495, hsa-miR-215, hsa-miR-26b*, hsa-miR-1253, hsa-miR-135a, hsa-miR-146a, hsa-miR-605, hsa-miR-1259, hsa-miR-633, hsa-miR-653, hsa-miR-888, hsa-miR-570, hsa-miR-124, hsa-miR-517*, hsa-miR-155, hsa-miR-548d-5p, hsa-miR-432, hsa-miR-331-5p, hsa-miR-144*, hsa-miR-20a*, hsa-miR-135b, hsa-miR-548j, hsa-miR-607, hsa-miR-10b*, hsa-miR-1206, hsa-miR-422a, hsa-miR-101, hsa-miR-539, hsa-miR-548a-3p, hsa-miR-146b-5p, hsa-miR-133b, hsa-miR-889, hsa-miR-548h, hsa-miR-92a, hsa-miR-381, hsa-miR-519e*, hsa-miR-150, hsa-miR-185, hsa-miR-877*, hsa-miR-522, hsa-miR-548c-3p, hsa-miR-106a*, hsa-miR-382, hsa-miR-548l, hsa-miR-506, hsa-miR-548e, hsa-miR-21, hsa-miR-655, hsa-miR-485-5p, hsa-miR-1278, hsa-miR-888*, hsa-miR-577, hsa-miR-300, hsa-miR-219-5p, hsa-miR-556-3p, hsa-miR-126*, hsa-miR-363, hsa-miR-509-5p, hsa-miR-944, hsa-miR-340, hsa-miR-499-5p, hsa-miR-369-3p, hsa-miR-218, hsa-miR-223*, hsa-miR-433, hsa-miR-154, hsa-miR-1205, hsa-miR-671-5p, hsa-miR-584.

Toll-like receptor adaptor molecule 2—TICAM2—NM_{—}021649—hsa-miR-576-3p, hsa-miR-376b, hsa-miR-877, hsa-miR-922, hsa-miR-216a, hsa-miR-181a, hsa-miR-302c*, hsa-miR-204, hsa-miR-377, hsa-miR-335, hsa-miR-511, hsa-miR-544, hsa-miR-26a-1*, hsa-miR-335*, hsa-miR-1237, hsa-miR-129-5p, hsa-miR-548n, hsa-miR-342-3p, hsa-miR-298, hsa-miR-587, hsa-miR-1303, hsa-miR-580, hsa-miR-660, hsa-miR-1246, hsa-miR-299-5p, hsa-miR-548p, hsa-miR-23a, hsa-miR-491-5p, hsa-let-7b*, hsa-miR-494, hsa-miR-29a*, hsa-miR-376a, hsa-miR-548f, hsa-miR-590-3p, hsa-miR-513c, hsa-miR-1253, hsa-miR-1200, hsa-miR-579, hsa-miR-26a-2*, hsa-miR-361-5p, hsa-miR-1262, hsa-miR-384, hsa-miR-654-3p, hsa-miR-21*, hsa-miR-624*, hsa-miR-517*, hsa-miR-588, hsa-miR-181d, hsa-miR-935, hsa-miR-9*, hsa-miR-1258, hsa-miR-578, hsa-miR-383, hsa-miR-1206, hsa-miR-23b, hsa-miR-539, hsa-miR-548a-3p, hsa-miR-381, hsa-miR-211, hsa-let-7f-1*, hsa-miR-1300, hsa-miR-181c, hsa-miR-659, hsa-miR-138-2*, hsa-miR-130a*, hsa-miR-106a*, hsa-miR-1264, hsa-miR-548k, hsa-miR-181b, hsa-miR-548e, hsa-miR-577, hsa-miR-300, hsa-miR-485-3p, hsa-let-7a*, hsa-miR-1272, hsa-miR-340, hsa-miR-219-2-3p, hsa-miR-433, hsa-miR-595.

KDEL (Lys-Asp-Glu-Leu) endoplasmic reticulum protein retention receptor 3—KDELR3—Variant 1—NM_{—}006855—hsa-miR-34c-5p, hsa-miR-380*, hsa-miR-19b-2*, hsa-miR-766, hsa-miR-181a, hsa-miR-513b, hsa-miR-302c*, hsa-miR-193a-5p, hsa-miR-563, hsa-miR-490-3p, hsa-miR-449a, hsa-miR-29b-1*, hsa-miR-19b, hsa-miR-1277, hsa-miR-19a*, hsa-miR-619, hsa-miR-875-3p, hsa-miR-19b-1*, hsa-miR-543, hsa-miR-449b, hsa-miR-151-3p, hsa-miR-146a*, hsa-miR-582-3p, hsa-miR-548o, hsa-miR-548f, hsa-miR-767-3p, hsa-miR-34a, hsa-miR-1323, hsa-miR-624*, hsa-miR-488, hsa-miR-940, hsa-miR-325, hsa-miR-1261, hsa-miR-432, hsa-miR-593, hsa-miR-34b*, hsa-miR-181d, hsa-miR-636, hsa-miR-578, hsa-miR-545*, hsa-miR-548a-3p, hsa-miR-381, hsa-miR-593*, hsa-miR-1300, hsa-miR-181c, hsa-miR-493, hsa-miR-205, hsa-miR-522, hsa-miR-431, hsa-miR-181b, hsa-miR-548e, hsa-miR-655, hsa-miR-137, hsa-miR-924, hsa-miR-19a, hsa-miR-300, hsa-miR-627, hsa-miR-767-5p, hsa-miR-218, hsa-miR-154, hsa-miR-1275, hsa-miR-1302 MIMAT0005890.

KDELR3—Variant 2—NM_{—}016657—hsa-miR-943, hsa-miR-432*.

Glucosamine-6-phosphate deaminase 1—GNPDA1—NM_{—}005471—hsa-miR-665, hsa-miR-637, hsa-miR-510, hsa-miR-200b, hsa-miR-34c-5p, hsa-miR-32, hsa-miR-645, hsa-miR-27b*, hsa-miR-22, hsa-miR-625, hsa-miR-650, hsa-miR-125a-5p, hsa-miR-337-5p, hsa-miR-28-3p, hsa-miR-532-3p, hsa-miR-638, hsa-miR-1304, hsa-miR-890, hsa-miR-376c, hsa-miR-490-3p, hsa-miR-516a-3p, hsa-miR-320c, hsa-miR-661, hsa-miR-486-3p, hsa-miR-449a, hsa-miR-1237, hsa-miR-561, hsa-miR-938, hsa-miR-128, hsa-miR-342-3p, hsa-miR-92b, hsa-miR-103, hsa-miR-1252, hsa-miR-129*, hsa-miR-367, hsa-miR-30b, hsa-miR-543, hsa-miR-25, hsa-miR-23a, hsa-miR-449b, hsa-miR-491-5p, hsa-miR-218-2*, hsa-miR-516b*, hsa-miR-30a, hsa-miR-200c, hsa-miR-220c, hsa-miR-1286, hsa-miR-765, hsa-miR-296-5p, hsa-miR-320d, hsa-miR-502-5p, hsa-miR-513c, hsa-miR-7-1*, hsa-miR-589*, hsa-miR-34a, hsa-miR-1243, hsa-miR-125b, hsa-miR-214*, hsa-miR-505*, hsa-miR-592, hsa-miR-429, hsa-miR-488, hsa-miR-769-3p, hsa-miR-450b-3p, hsa-miR-198, hsa-miR-514, hsa-miR-200a, hsa-miR-30d, hsa-miR-629*, hsa-miR-518c*, hsa-miR-23b, hsa-miR-1263, hsa-miR-539, hsa-miR-516b, hsa-miR-330-5p, hsa-miR-141, hsa-miR-92a, hsa-miR-744*, hsa-miR-361-3p, hsa-miR-150, hsa-miR-30c, hsa-miR-760, hsa-miR-221*, hsa-miR-185, hsa-miR-378*, hsa-miR-556-5p, hsa-miR-640, hsa-miR-130a*, hsa-miR-758, hsa-miR-320a, hsa-miR-1225-5p, hsa-miR-129-3p, hsa-miR-1827, hsa-miR-320b, hsa-miR-122, hsa-miR-7-2*, hsa-miR-107, hsa-miR-363, hsa-miR-574-5p, hsa-miR-296-3p, hsa-miR-367*, hsa-miR-326, hsa-miR-223*, hsa-miR-1275, hsa-miR-671-5p, hsa-miR-30e.

Thioesterase superfamily member 2—THEM2—NM_{—}018473—hsa-miR-19b-2*, hsa-miR-29b-1*, hsa-miR-19a*, hsa-miR-19b-1*, hsa-miR-664, hsa-miR-26b*.

Debranching enzyme homolog 1—DBR1—NM_{—}016216—hsa-miR-32, hsa-miR-500, hsa-miR-20b*, hsa-miR-650, hsa-miR-550*, hsa-miR-374b, hsa-miR-96, hsa-miR-490-3p, hsa-miR-320c, hsa-miR-142-5p, hsa-miR-658, hsa-miR-128, hsa-miR-508-5p, hsa-miR-92b, hsa-miR-548p, hsa-miR-367, hsa-miR-25*, hsa-miR-524-5p, hsa-miR-25, hsa-miR-10a, hsa-miR-33a*, hsa-miR-302f, hsa-let-7b*, hsa-miR-494, hsa-miR-616*, hsa-miR-552, hsa-miR-373*, hsa-miR-362-5p, hsa-miR-26b, hsa-miR-320d, hsa-miR-495, hsa-miR-1271, hsa-miR-1297, hsa-miR-520f, hsa-miR-768-5p, hsa-miR-1259, hsa-miR-200c*, hsa-miR-122*, hsa-miR-940, hsa-miR-432, hsa-miR-636, hsa-miR-1260, hsa-miR-571, hsa-miR-92a, hsa-let-7f-1*, hsa-miR-1224-3p, hsa-miR-30b*, hsa-miR-320a, hsa-miR-10b, hsa-miR-339-5p, hsa-miR-1827, hsa-miR-548c-3p, hsa-miR-1279, hsa-miR-656, hsa-miR-320b, hsa-miR-1264, hsa-miR-297, hsa-miR-485-5p, hsa-miR-577, hsa-miR-377*, hsa-miR-374a, hsa-miR-1244, hsa-miR-126*, hsa-miR-363, hsa-let-7a*, hsa-miR-340, hsa-miR-369-3p, hsa-miR-26a, hsa-miR-520d-5p.

Solute carrier family 38, member 9—SLC38A9—NM_{—}173514—hsa-miR-509-3-5p, hsa-miR-16, hsa-miR-125a-5p, hsa-miR-548m, hsa-miR-129-5p, hsa-miR-623, hsa-miR-103, hsa-miR-580, hsa-miR-548p, hsa-miR-338-5p, hsa-miR-202, hsa-miR-494, hsa-miR-616*, hsa-miR-373*, hsa-miR-628-3p, hsa-miR-15b, hsa-miR-495, hsa-miR-936, hsa-miR-125b, hsa-miR-384, hsa-miR-220b, hsa-miR-195, hsa-miR-323-3p, hsa-miR-607, hsa-miR-200a, hsa-miR-141, hsa-miR-583, hsa-miR-138-2*, hsa-miR-522, hsa-miR-424, hsa-miR-107, hsa-miR-15a, hsa-miR-601, hsa-miR-338-3p, hsa-miR-509-5p, hsa-miR-499-5p, hsa-miR-140-3p, hsa-miR-497, hsa-miR-433, hsa-miR-1205.

N-acetyltrans-ferase 10 (GCN5-related)—NAT10—NM_{—}024662—hsa-miR-576-3p, hsa-miR-194*, hsa-miR-518d-5p, hsa-miR-490-5p, hsa-miR-34c-5p, hsa-miR-16, hsa-miR-519a*, hsa-miR-141*, hsa-miR-522*, hsa-miR-520g, hsa-miR-320c, hsa-miR-148a*, hsa-miR-449a, hsa-miR-378, hsa-miR-330-3p, hsa-miR-660, hsa-miR-548p, hsa-miR-449b, hsa-miR-212, hsa-miR-92b*, hsa-miR-1269, hsa-miR-15b, hsa-miR-320d, hsa-miR-132, hsa-miR-1291, hsa-miR-34a, hsa-miR-921, hsa-miR-376a*, hsa-miR-147, hsa-miR-220b, hsa-miR-505*, hsa-miR-1224-5p, hsa-miR-184, hsa-miR-518f*, hsa-miR-1285, hsa-miR-195, hsa-miR-545, hsa-miR-612, hsa-miR-520c-5p, hsa-miR-422a, hsa-miR-526b, hsa-miR-518e*, hsa-miR-593*, hsa-miR-526a, hsa-miR-523*, hsa-miR-149, hsa-miR-520h, hsa-miR-320a, hsa-miR-320b, hsa-miR-424, hsa-miR-15a, hsa-miR-455-3p, hsa-miR-519b-5p, hsa-miR-497, hsa-miR-519c-5p, hsa-miR-1302.

Chromosome 16 open reading frame 33—C16orf33—NM_{—}024571—hsa-miR-194*, hsa-miR-661, hsa-miR-1226*, hsa-miR-892b, hsa-miR-1231, hsa-miR-370, hsa-miR-660, hsa-miR-10a, hsa-miR-296-5p, hsa-miR-1200, hsa-miR-488, hsa-miR-935, hsa-miR-554, hsa-miR-516b, hsa-miR-361-3p, hsa-miR-628-5p, hsa-miR-583, hsa-miR-611, hsa-miR-149, hsa-miR-10b, hsa-miR-339-5p, hsa-miR-296-3p, hsa-let-7f-2*, hsa-miR-346.

Lactate dehydrogenase A—LDHA-NM_{—}005566—hsa-miR-34c-5p, hsa-miR-219-1-3p, hsa-miR-1290, hsa-miR-890, hsa-miR-449a, hsa-miR-30b, hsa-miR-449b, hsa-miR-410, hsa-miR-421, hsa-miR-641, hsa-miR-30a, hsa-miR-489, hsa-miR-1208, hsa-miR-34a, hsa-miR-1243, hsa-miR-505*, hsa-miR-105, hsa-miR-30d, hsa-miR-383, hsa-miR-491-3p, hsa-miR-33b, hsa-miR-203, hsa-miR-30c, hsa-miR-501-5p, hsa-miR-138-2*, hsa-miR-149, hsa-miR-297, hsa-miR-577, hsa-miR-33a, hsa-miR-409-3p, hsa-miR-455-3p, hsa-miR-338-3p, hsa-miR-1265, hsa-miR-193b*, hsa-miR-30e.

Major histocompatib-ility complex, class I-related—MR1—NM_{—}001531—hsa-miR-877, hsa-miR-645, hsa-miR-320c, hsa-miR-885-5p, hsa-miR-875-3p, hsa-miR-212, hsa-miR-320d, hsa-miR-132, hsa-miR-146a, hsa-miR-498, hsa-miR-505*, hsa-miR-146b-5p, hsa-miR-567, hsa-miR-1185, hsa-miR-583, hsa-miR-320a, hsa-miR-320b, hsa-miR-297, hsa-miR-455-3p, hsa-miR-367*, hsa-miR-1275.

Leucyl-tRNA synthetase—LARS—NM_{—}020117—hsa-miR-637, hsa-miR-520b, hsa-miR-515-5p, hsa-miR-625, hsa-miR-520g, hsa-miR-222*, hsa-miR-551b*, hsa-miR-320c, hsa-miR-520a-3p, hsa-miR-148a*, hsa-miR-142-5p, hsa-miR-302e, hsa-miR-134, hsa-miR-342-3p, hsa-miR-130b, hsa-miR-1246, hsa-miR-365, hsa-miR-301b, hsa-miR-302c, hsa-miR-548d-3p, hsa-miR-646, hsa-let-7b*, hsa-miR-411, hsa-miR-371-5p, hsa-miR-373, hsa-miR-1207-3p, hsa-miR-130a, hsa-miR-1286, hsa-miR-32*, hsa-miR-302d, hsa-miR-320d, hsa-miR-936, hsa-miR-186*, hsa-miR-520c-3p, hsa-miR-498, hsa-miR-633, hsa-miR-302a, hsa-miR-125a-3p, hsa-miR-512-3p, hsa-miR-520d-3p, hsa-miR-519d, hsa-miR-9*, hsa-miR-520e, hsa-miR-93, hsa-miR-634, hsa-miR-301a, hsa-miR-526b*, hsa-miR-744*, hsa-miR-519e*, hsa-let-7f-1*, hsa-miR-432*, hsa-miR-150, hsa-miR-205, hsa-miR-659, hsa-miR-20b, hsa-miR-378*, hsa-miR-1224-3p, hsa-miR-106a, hsa-miR-483-5p, hsa-miR-520h, hsa-miR-320a, hsa-miR-17, hsa-miR-106a*, hsa-miR-182, hsa-miR-320b, hsa-miR-454, hsa-miR-1264, hsa-miR-548k, hsa-miR-548l, hsa-miR-302b, hsa-miR-888*, hsa-miR-564, hsa-miR-20a, hsa-miR-183, hsa-let-7a*, hsa-miR-338-3p, hsa-miR-340, hsa-miR-193b*, hsa-miR-106b, hsa-miR-372.

GTP binding protein 1—GTPBP1—NM_{—}004286—hsa-miR-194*, hsa-miR-132*, hsa-miR-453, hsa-miR-637, hsa-miR-490-5p, hsa-miR-1266, hsa-miR-16, hsa-miR-20b*, hsa-miR-515-5p, hsa-miR-380*, hsa-miR-922, hsa-miR-138, hsa-miR-766, hsa-miR-650, hsa-miR-1276, hsa-miR-374b, hsa-miR-182*, hsa-miR-125a-5p, hsa-miR-483-3p, hsa-miR-193a-5p, hsa-miR-563, hsa-miR-214, hsa-miR-890, hsa-miR-490-3p, hsa-miR-92a-2*, hsa-miR-363*, hsa-miR-575, hsa-miR-320c, hsa-miR-511, hsa-miR-661, hsa-miR-544, hsa-miR-143, hsa-miR-1226*, hsa-miR-1237, hsa-miR-658, hsa-let-7c*, hsa-miR-886-3p, hsa-miR-1825, hsa-miR-1268, hsa-miR-298, hsa-miR-508-5p, hsa-miR-619, hsa-miR-370, hsa-miR-1207-5p, hsa-miR-623, hsa-miR-875-3p, hsa-miR-148a, hsa-miR-25*, hsa-miR-23a, hsa-miR-365, hsa-miR-337-3p, hsa-miR-10a, hsa-miR-33 a*, hsa-miR-491-5p, hsa-miR-218-2*, hsa-miR-151-3p, hsa-miR-146a*, hsa-miR-590-5p, hsa-miR-489, hsa-miR-324-3p, hsa-miR-1, hsa-miR-581, hsa-miR-1207-3p, hsa-miR-145, hsa-miR-616*, hsa-miR-874, hsa-miR-148b, hsa-miR-373*, hsa-miR-548f, hsa-miR-220c, hsa-miR-765, hsa-miR-624, hsa-miR-296-5p, hsa-miR-15b, hsa-miR-1324, hsa-miR-320d, hsa-miR-590-3p, hsa-miR-495, hsa-miR-29c, hsa-miR-589*, hsa-miR-1291, hsa-miR-1200, hsa-miR-29b, hsa-miR-135a, hsa-miR-605, hsa-miR-768-5p, hsa-miR-186*, hsa-miR-608, hsa-miR-27a*, hsa-miR-18b, hsa-miR-1226, hsa-miR-185*, hsa-miR-125b, hsa-miR-505*, hsa-miR-596, hsa-miR-29a, hsa-miR-940, hsa-miR-34b, hsa-miR-34b*, hsa-miR-1285, hsa-miR-195, hsa-miR-92a-1*, hsa-miR-612, hsa-miR-135b, hsa-miR-664*, hsa-miR-629*, hsa-miR-1260, hsa-miR-942, hsa-miR-152, hsa-miR-23b, hsa-miR-23a*, hsa-miR-1274a, hsa-miR-545*, hsa-miR-548a-3p, hsa-miR-23b*, hsa-miR-634, hsa-miR-33b*, hsa-miR-149*, hsa-miR-22*, hsa-miR-708*, hsa-miR-597, hsa-miR-519e*, hsa-miR-593*, hsa-miR-432*, hsa-miR-18a, hsa-miR-221*, hsa-miR-185, hsa-miR-378*, hsa-miR-1321, hsa-miR-100*, hsa-miR-149, hsa-miR-18a*, hsa-miR-30b*, hsa-miR-130a*, hsa-miR-519e, hsa-miR-550, hsa-miR-320a, hsa-miR-10b, hsa-miR-339-5p, hsa-miR-1827, hsa-miR-24, hsa-miR-135a*, hsa-miR-196a*, hsa-miR-663b, hsa-miR-500*, hsa-miR-320b, hsa-miR-1274b, hsa-miR-675, hsa-miR-424, hsa-miR-654-5p, hsa-miR-626, hsa-miR-1283, hsa-miR-187*, hsa-miR-548e, hsa-miR-21, hsa-miR-485-5p, hsa-miR-1278, hsa-miR-331-3p, hsa-miR-206, hsa-miR-196a, hsa-miR-377*, hsa-miR-15a, hsa-miR-632, hsa-miR-219-5p, hsa-miR-374a, hsa-miR-423-5p, hsa-miR-455-3p, hsa-miR-196b, hsa-miR-1272, hsa-miR-515-3p, hsa-miR-497, hsa-miR-296-3p, hsa-miR-1197, hsa-miR-346, hsa-miR-505, hsa-miR-541, hsa-miR-135b*, hsa-miR-1293, hsa-miR-613, hsa-miR-671-5p, hsa-miR-340*.

Protease, serine, 16—PRSS16—NM_{—}005865—hsa-miR-140-5p, hsa-miR-665, hsa-miR-1266, hsa-miR-542-3p, hsa-miR-922, hsa-miR-181a, hsa-miR-454*, hsa-miR-195*, hsa-miR-302c*, hsa-miR-214, hsa-miR-16-2*, hsa-miR-320c, hsa-miR-15b*, hsa-miR-28-5p, hsa-miR-19b, hsa-miR-129-5p, hsa-miR-130b, hsa-miR-587, hsa-miR-657, hsa-miR-1252, hsa-miR-580, hsa-miR-148a, hsa-miR-600, hsa-miR-524-5p, hsa-miR-543, hsa-miR-23a, hsa-miR-301b, hsa-miR-641, hsa-miR-708, hsa-miR-130a, hsa-miR-552, hsa-miR-148b, hsa-miR-320d, hsa-miR-591, hsa-miR-767-3p, hsa-miR-1291, hsa-miR-1226, hsa-miR-1224-5p, hsa-miR-624*, hsa-miR-92a-1*, hsa-miR-198, hsa-miR-545, hsa-miR-181d, hsa-miR-192*, hsa-miR-152, hsa-miR-23b, hsa-miR-539, hsa-miR-634, hsa-miR-149*, hsa-miR-301a, hsa-miR-571, hsa-miR-593*, hsa-miR-1300, hsa-miR-181c, hsa-miR-224, hsa-miR-185, hsa-miR-130a*, hsa-miR-320a, hsa-miR-7, hsa-miR-320b, hsa-miR-454, hsa-miR-654-5p, hsa-miR-181b, hsa-miR-137, hsa-miR-577, hsa-miR-19a, hsa-miR-338-3p, hsa-miR-1265, hsa-miR-1197, hsa-miR-541, hsa-miR-520d-5p.

WAP four-disulfide core domain 2—WFDC2—NM_{—}006103—hsa-miR-140-5p, hsa-miR-129-5p, hsa-miR-767-3p, hsa-miR-92a-1*, hsa-miR-149*, hsa-miR-654-5p, hsa-miR-541.

Aurora kinase B—AURKB—NM_{—}004217—hsa-miR-502-3p, hsa-miR-509-3-5p, hsa-miR-490-5p, hsa-miR-493*, hsa-miR-138, hsa-let-7d, hsa-miR-766, hsa-miR-550*, hsa-miR-551b*, hsa-miR-647, hsa-miR-1248, hsa-miR-335, hsa-miR-320c, hsa-miR-661, hsa-miR-31*, hsa-miR-31, hsa-miR-148a*, hsa-miR-142-5p, hsa-miR-379*, hsa-miR-190, hsa-miR-548n, hsa-miR-603, hsa-miR-508-5p, hsa-let-7e, hsa-miR-103, hsa-miR-548p, hsa-miR-33a*, hsa-miR-302f, hsa-miR-520a-5p, hsa-miR-410, hsa-miR-421, hsa-miR-646, hsa-miR-525-5p, hsa-miR-622, hsa-miR-1227, hsa-miR-145, hsa-miR-616*, hsa-miR-373*, hsa-let-7f, hsa-miR-32*, hsa-miR-765, hsa-miR-320d, hsa-miR-29c, hsa-miR-7-1*, hsa-miR-1208, hsa-miR-589*, hsa-miR-26b*, hsa-miR-1253, hsa-miR-190b, hsa-miR-29b, hsa-miR-579, hsa-miR-411*, hsa-miR-200c*, hsa-miR-147, hsa-miR-633, hsa-miR-1224-5p, hsa-miR-888, hsa-miR-29a, hsa-miR-503, hsa-miR-488, hsa-let-7g, hsa-miR-1285, hsa-miR-15a*, hsa-miR-612, hsa-miR-9*, hsa-miR-607, hsa-miR-200a, hsa-miR-548b-3p, hsa-let-7i, hsa-miR-1255a, hsa-miR-634, hsa-miR-526b, hsa-let-7b, hsa-miR-141, hsa-miR-223, hsa-let-7a, hsa-miR-98, hsa-miR-203, hsa-miR-583, hsa-miR-17*, hsa-miR-30b*, hsa-miR-758, hsa-miR-320a, hsa-let-7c, hsa-miR-1228, hsa-miR-24, hsa-miR-548c-3p, hsa-miR-1279, hsa-miR-320b, hsa-miR-548l, hsa-miR-1283, hsa-miR-485-5p, hsa-miR-7-2*, hsa-miR-107, hsa-miR-338-3p, hsa-miR-509-5p, hsa-miR-944, hsa-miR-501-3p, hsa-miR-16-1*, hsa-miR-649, hsa-miR-1183, hsa-miR-1255b, hsa-miR-1205.

3-hydroxybutyrate dehydrogenase, type 2—BDH2—NM_{—}020139—hsa-miR-376b, hsa-miR-509-3-5p, hsa-miR-642, hsa-miR-27b*, hsa-miR-130b*, hsa-miR-648, hsa-miR-1276, hsa-miR-1184, hsa-miR-520g, hsa-miR-519b-3p, hsa-miR-195*, hsa-miR-302c*, hsa-miR-487a, hsa-miR-193a-5p, hsa-miR-1304, hsa-miR-92a-2*, hsa-miR-16-2*, hsa-miR-148a*, hsa-miR-143, hsa-miR-26a-1*, hsa-miR-142-5p, hsa-miR-335*, hsa-miR-519c-3p, hsa-miR-129-5p, hsa-miR-1178, hsa-miR-217, hsa-miR-519a, hsa-miR-1182, hsa-miR-651, hsa-miR-338-5p, hsa-miR-524-5p, hsa-miR-143*, hsa-miR-186, hsa-miR-33a*, hsa-miR-590-5p, hsa-miR-371-5p, hsa-miR-489, hsa-miR-376a, hsa-miR-874, hsa-miR-26b, hsa-miR-573, hsa-miR-32*, hsa-miR-590-3p, hsa-miR-495, hsa-miR-1297, hsa-miR-7-1*, hsa-miR-635, hsa-miR-767-3p, hsa-miR-589*, hsa-miR-579, hsa-miR-26a-2*, hsa-miR-605, hsa-miR-1243, hsa-miR-186*, hsa-miR-509-3p, hsa-miR-1262, hsa-miR-384, hsa-miR-34b, hsa-miR-1270, hsa-miR-18b*, hsa-miR-519d, hsa-miR-200a, hsa-miR-93, hsa-miR-554, hsa-miR-1260, hsa-miR-620, hsa-miR-141, hsa-miR-526b*, hsa-miR-1185, hsa-miR-150, hsa-miR-1284, hsa-miR-20b, hsa-miR-556-5p, hsa-miR-106a, hsa-miR-1245, hsa-miR-30b*, hsa-miR-520h, hsa-miR-431, hsa-miR-17, hsa-miR-106a*, hsa-miR-656, hsa-miR-548l, hsa-miR-21, hsa-miR-485-5p, hsa-miR-888*, hsa-miR-7-2*, hsa-miR-377*, hsa-miR-601, hsa-miR-20a, hsa-miR-425, hsa-miR-126*, hsa-miR-509-5p, hsa-miR-944, hsa-miR-106b, hsa-miR-505, hsa-miR-26a, hsa-miR-142-3p, hsa-miR-520d-5p, hsa-miR-154*.

Dehydrogenase/reductase (SDR family) member 4—DHRS4—NM_{—}021004—hsa-miR-194*, hsa-miR-557, hsa-miR-665, hsa-miR-1266, hsa-miR-922, hsa-miR-513a-5p, hsa-miR-214, hsa-miR-507, hsa-miR-28-5p, hsa-miR-508-5p, hsa-miR-1207-5p, hsa-miR-1287, hsa-miR-379, hsa-miR-208a, hsa-miR-708, hsa-miR-29a*, hsa-miR-220c, hsa-miR-29c, hsa-miR-29b, hsa-miR-579, hsa-miR-605, hsa-miR-1262, hsa-miR-214*, hsa-miR-29a, hsa-miR-450b-5p, hsa-miR-20a*, hsa-miR-452*, hsa-miR-10b*, hsa-miR-942, hsa-miR-1257, hsa-miR-208b, hsa-miR-663b, hsa-miR-500*, hsa-miR-382, hsa-miR-626, hsa-miR-512-5p.

Abhydrolase domain containing 14B—ABHD14B—NM_{—}032750—hsa-miR-637, hsa-miR-520b, hsa-miR-380, hsa-miR-562, hsa-let-7d, hsa-miR-1184, hsa-miR-615-5p, hsa-miR-204, hsa-miR-1289, hsa-miR-532-3p, hsa-miR-30c-1*, hsa-miR-92a-2*, hsa-miR-575, hsa-miR-320c, hsa-miR-520a-3p, hsa-miR-661, hsa-miR-302e, hsa-let-7c*, hsa-miR-298, hsa-let-7e, hsa-miR-1252, hsa-miR-1246, hsa-miR-576-5p, hsa-miR-30c-2*, hsa-miR-302c, hsa-miR-491-5p, hsa-miR-520a-5p, hsa-miR-145*, hsa-miR-525-5p, hsa-miR-373, hsa-miR-581, hsa-let-7f, hsa-miR-302d, hsa-miR-765, hsa-miR-320d, hsa-miR-1253, hsa-miR-1200, hsa-miR-520f, hsa-miR-147, hsa-miR-520c-3p, hsa-miR-518a-5p, hsa-miR-517b, hsa-miR-302a, hsa-let-7g, hsa-miR-940, hsa-miR-1294, hsa-miR-588, hsa-miR-1285, hsa-miR-512-3p, hsa-miR-520d-3p, hsa-miR-198, hsa-miR-612, hsa-miR-299-3p, hsa-miR-636, hsa-miR-520e, hsa-miR-1258, hsa-miR-548b-3p, hsa-miR-1260, hsa-let-7i, hsa-miR-609, hsa-let-7b, hsa-miR-149*, hsa-miR-328, hsa-let-7a, hsa-miR-98, hsa-miR-211, hsa-miR-583, hsa-miR-527, hsa-miR-185, hsa-miR-100*, hsa-miR-1224-3p, hsa-miR-149, hsa-miR-30b*, hsa-miR-320a, hsa-miR-555, hsa-let-7c, hsa-miR-663b, hsa-miR-320b, hsa-miR-297, hsa-miR-302b, hsa-miR-455-3p, hsa-miR-649, hsa-miR-372, hsa-miR-367*.

EF-hand calcium binding domain 4A—EFCAB4A—NM_{—}173584—hsa-miR-194*, hsa-miR-637, hsa-miR-645, hsa-miR-16, hsa-miR-1184, hsa-miR-1301, hsa-miR-483-3p, hsa-miR-668, hsa-miR-214, hsa-miR-661, hsa-miR-93*, hsa-miR-148a, hsa-miR-615-3p, hsa-miR-143*, hsa-miR-148b, hsa-miR-15b, hsa-miR-1200, hsa-miR-185*, hsa-miR-596, hsa-miR-940, hsa-miR-588, hsa-miR-195, hsa-miR-504, hsa-miR-152, hsa-miR-551a, hsa-miR-205, hsa-miR-424, hsa-miR-654-5p, hsa-miR-15a, hsa-miR-193b*, hsa-miR-887, hsa-miR-497, hsa-miR-551b, hsa-miR-602, hsa-miR-541.

Enoyl Coenzyme A hydratase domain containing 2—ECHDC2—NM_{—}018281—hsa-miR-141*, hsa-miR-218-1*, hsa-miR-127-5p, hsa-miR-330-3p, hsa-miR-1237, hsa-miR-576-5p, hsa-miR-362-5p, hsa-miR-26b, hsa-miR-1297, hsa-miR-588, hsa-miR-516b, hsa-miR-203, hsa-miR-659, hsa-miR-1272, hsa-miR-26a.

Golgi autoantigen, golgin subfamily a,8B—GOLGA8B—NM_{—}001023567—hsa-miR-557, hsa-miR-200b, hsa-miR-520b, hsa-miR-32, hsa-miR-130b*, hsa-miR-192, hsa-miR-1184, hsa-miR-374b, hsa-miR-1301, hsa-miR-181a, hsa-miR-551b*, hsa-miR-513b, hsa-miR-487a, hsa-miR-193a-5p, hsa-miR-96, hsa-miR-532-3p, hsa-miR-377, hsa-miR-548a-5p, hsa-miR-559, hsa-miR-1267, hsa-miR-548b-5p, hsa-miR-520a-3p, hsa-miR-513a-3p, hsa-miR-507, hsa-miR-1226*, hsa-miR-302a*, hsa-miR-19b, hsa-miR-302e, hsa-miR-548c-5p, hsa-miR-603, hsa-miR-128, hsa-miR-92b, hsa-miR-130b, hsa-miR-651, hsa-miR-148a, hsa-miR-586, hsa-miR-367, hsa-miR-582-5p, hsa-miR-524-5p, hsa-miR-30b, hsa-miR-543, hsa-miR-25, hsa-miR-23a, hsa-miR-301b, hsa-miR-1305, hsa-miR-33a*, hsa-miR-302c, hsa-miR-548i, hsa-miR-646, hsa-miR-153, hsa-miR-30a*, hsa-miR-30a, hsa-miR-373, hsa-miR-200c, hsa-miR-130a, hsa-miR-145, hsa-miR-148b, hsa-let-7i*, hsa-miR-664, hsa-miR-1269, hsa-miR-302d, hsa-miR-628-3p, hsa-miR-590-3p, hsa-miR-1271, hsa-miR-215, hsa-miR-589*, hsa-miR-936, hsa-miR-520c-3p, hsa-miR-518a-5p, hsa-miR-498, hsa-miR-1226, hsa-miR-144, hsa-miR-302a, hsa-miR-30d*, hsa-miR-30e*, hsa-miR-429, hsa-miR-105, hsa-miR-548d-5p, hsa-miR-1270, hsa-miR-520d-3p, hsa-miR-331-5p, hsa-miR-450b-5p, hsa-miR-181d, hsa-miR-935, hsa-miR-548j, hsa-miR-323-3p, hsa-miR-607, hsa-miR-520e, hsa-miR-30d, hsa-miR-629, hsa-miR-934, hsa-miR-548b-3p, hsa-miR-1260, hsa-miR-152, hsa-miR-23b, hsa-miR-101, hsa-miR-1257, hsa-miR-634, hsa-miR-526b, hsa-miR-620, hsa-miR-139-5p, hsa-miR-301a, hsa-miR-223, hsa-miR-203, hsa-miR-548h, hsa-miR-92a, hsa-miR-708*, hsa-miR-597, hsa-miR-1238, hsa-miR-30c, hsa-miR-181c, hsa-miR-224, hsa-miR-527, hsa-miR-877*, hsa-miR-18a*, hsa-miR-130a*, hsa-miR-202*, hsa-miR-1249, hsa-miR-1279, hsa-miR-656, hsa-miR-182, hsa-miR-454, hsa-miR-1264, hsa-miR-302b, hsa-miR-181b, hsa-miR-655, hsa-miR-485-5p, hsa-miR-137, hsa-miR-196 a, hsa-miR-19a, hsa-miR-485-3p, hsa-miR-374a, hsa-miR-601, hsa-miR-876-3p, hsa-miR-196b, hsa-miR-363, hsa-miR-1265, hsa-miR-219-2-3p, hsa-miR-369-3p, hsa-miR-16-1*, hsa-miR-372, hsa-miR-154, hsa-miR-520d-5p, hsa-miR-1302, hsa-miR-154*, hsa-miR-30e.

ATPase type 13A3—ATP13A3—NM_{—}024524—hsa-miR-376b, hsa-miR-200b, hsa-miR-34c-5p, hsa-miR-380, hsa-miR-16, hsa-miR-513a-5p, hsa-miR-133a, hsa-miR-519b-3p, hsa-miR-182*, hsa-miR-195*, hsa-miR-125a-5p, hsa-miR-548g, hsa-let-7g*, hsa-miR-532-3p, hsa-miR-548a-5p, hsa-miR-376c, hsa-miR-559, hsa-miR-548b-5p, hsa-miR-452, hsa-miR-16-2*, hsa-miR-320c, hsa-miR-511, hsa-miR-15b*, hsa-miR-31, hsa-miR-513a-3p, hsa-miR-142-5p, hsa-miR-335*, hsa-miR-449a, hsa-miR-190, hsa-miR-302a*, hsa-miR-519c-3p, hsa-miR-129-5p, hsa-miR-194, hsa-miR-548n, hsa-miR-892b, hsa-miR-548c-5p, hsa-miR-519a, hsa-miR-130b, hsa-miR-1287, hsa-miR-875-3p, hsa-miR-103, hsa-miR-1246, hsa-miR-600, hsa-miR-338-5p, hsa-miR-543, hsa-miR-34a*, hsa-miR-186, hsa-miR-365, hsa-miR-301b, hsa-miR-449b, hsa-miR-618, hsa-miR-33a*, hsa-miR-548i, hsa-miR-606, hsa-miR-520a-5p, hsa-miR-548d-3p, hsa-let-7b*, hsa-miR-151-3p, hsa-miR-153, hsa-miR-525-5p, hsa-miR-494, hsa-miR-30a*, hsa-miR-376a, hsa-miR-200c, hsa-miR-130a, hsa-miR-616*, hsa-miR-373*, hsa-miR-548f, hsa-miR-1286, hsa-miR-664, hsa-miR-32*, hsa-miR-532-5p, hsa-miR-15b, hsa-miR-320d, hsa-miR-590-3p, hsa-miR-495, hsa-miR-513c, hsa-miR-7-1*, hsa-miR-1208, hsa-miR-589, hsa-miR-1253, hsa-miR-190b, hsa-miR-1200, hsa-miR-34a, hsa-miR-643, hsa-miR-579, hsa-miR-361-5p, hsa-miR-1243, hsa-miR-498, hsa-miR-125b, hsa-miR-384, hsa-miR-653, hsa-miR-505*, hsa-miR-888, hsa-miR-30d*, hsa-miR-802, hsa-miR-568, hsa-miR-30e*, hsa-miR-21*, hsa-miR-429, hsa-miR-570, hsa-miR-105, hsa-miR-517*, hsa-miR-548d-5p, hsa-miR-34b, hsa-miR-1261, hsa-miR-1270, hsa-miR-34b*, hsa-miR-195, hsa-miR-9*, hsa-miR-548j, hsa-miR-607, hsa-miR-1258, hsa-miR-629*, hsa-miR-383, hsa-miR-155*, hsa-miR-518c*, hsa-miR-1260, hsa-miR-548a-3p, hsa-miR-526b, hsa-miR-133b, hsa-miR-620, hsa-miR-301a, hsa-miR-548h, hsa-miR-625*, hsa-miR-708*, hsa-let-7f-1*, hsa-miR-181a-2*, hsa-miR-760, hsa-miR-640, hsa-miR-100*, hsa-miR-1224-3p, hsa-miR-1245, hsa-miR-149, hsa-miR-320a, hsa-miR-486-5p, hsa-miR-202*, hsa-miR-1228, hsa-miR-1299, hsa-miR-548c-3p, hsa-miR-135a*, hsa-miR-1279, hsa-miR-656, hsa-miR-182, hsa-miR-320b, hsa-miR-454, hsa-miR-297, hsa-miR-548l, hsa-miR-424, hsa-miR-1283, hsa-miR-548e, hsa-miR-655, hsa-miR-137, hsa-miR-7-2*, hsa-miR-107, hsa-miR-15a, hsa-miR-409-3p, hsa-miR-183, hsa-let-7a*, hsa-miR-340, hsa-miR-497, hsa-miR-296-3p, hsa-let-7f-2*, hsa-miR-16-1*, hsa-miR-223*.

Prolyl endopeptidase-like—PREPL—Variant C, Variant D and Variant E—NM_{—}006036, NM_{—}001042386 and NM_{—}001042385—hsa-miR-194*, hsa-miR-376b, hsa-miR-877, hsa-miR-1254, hsa-miR-642, hsa-miR-500, hsa-miR-27b*, hsa-miR-19b-2*, hsa-miR-875-5p, hsa-miR-150*, hsa-miR-551b*, hsa-let-7g*, hsa-miR-548a-5p, hsa-miR-559, hsa-miR-1248, hsa-miR-138-1*, hsa-miR-548b-5p, hsa-miR-511, hsa-miR-31, hsa-miR-143, hsa-miR-379*, hsa-miR-29b-1*, hsa-miR-28-5p, hsa-miR-129-5p, hsa-miR-548n, hsa-miR-548c-5p, hsa-miR-19a*, hsa-miR-128, hsa-miR-199a-5p, hsa-miR-1287, hsa-miR-580, hsa-miR-548p, hsa-miR-586, hsa-miR-621, hsa-miR-338-5p, hsa-miR-19b-1*, hsa-miR-524-5p, hsa-miR-30b, hsa-miR-543, hsa-miR-618, hsa-miR-1305, hsa-miR-33a*, hsa-miR-548i, hsa-miR-708, hsa-miR-590-5p, hsa-miR-494, hsa-miR-411, hsa-miR-30a*, hsa-miR-30a, hsa-miR-29a*, hsa-miR-376a, hsa-miR-1286, hsa-miR-26b, hsa-miR-569, hsa-miR-32*, hsa-miR-590-3p, hsa-miR-495, hsa-miR-513c, hsa-miR-1297, hsa-miR-7-1*, hsa-miR-1208, hsa-miR-767-3p, hsa-miR-589*, hsa-miR-26b*, hsa-miR-135a, hsa-miR-411*, hsa-miR-1259, hsa-miR-185*, hsa-miR-384, hsa-miR-888, hsa-miR-30d*, hsa-miR-654-3p, hsa-miR-30e*, hsa-miR-21 *, hsa-miR-624*, hsa-miR-488, hsa-miR-105, hsa-miR-508-3p, hsa-miR-940, hsa-miR-548d-5p, hsa-miR-210, hsa-miR-34b*, hsa-miR-935, hsa-miR-135b, hsa-miR-548j, hsa-miR-200a, hsa-miR-30d, hsa-miR-192*, hsa-miR-934, hsa-miR-545*, hsa-miR-149*, hsa-miR-302d*, hsa-miR-141, hsa-miR-548h, hsa-miR-381, hsa-miR-744*, hsa-miR-432*, hsa-miR-30c, hsa-miR-451, hsa-miR-583, hsa-miR-501-5p, hsa-miR-199b-5p, hsa-miR-205, hsa-miR-185, hsa-miR-556-5p, hsa-miR-1321, hsa-miR-1827, hsa-miR-548c-3p, hsa-miR-196a*, hsa-miR-1279, hsa-miR-656, hsa-miR-182, hsa-miR-1283, hsa-miR-21, hsa-miR-655, hsa-miR-7-2*, hsa-miR-300, hsa-miR-485-3p, hsa-miR-632, hsa-miR-944, hsa-miR-499-5p, hsa-miR-1197, hsa-miR-218, hsa-miR-302b*, hsa-miR-367*, hsa-miR-26a, hsa-miR-520d-5p, hsa-miR-512-5p, hsa-miR-30e.

Cytochrome P450, family 4, subfamily V, polypeptide 2—CYP4V2—NM_{—}207352—hsa-miR-194*, hsa-miR-510, hsa-miR-200b, hsa-miR-520b, hsa-miR-20b*, hsa-miR-130b*, hsa-miR-766, hsa-miR-9, hsa-miR-374b, hsa-miR-101*, hsa-miR-519b-3p, hsa-miR-125a-5p, hsa-miR-483-3p, hsa-miR-548g, hsa-miR-28-3p, hsa-miR-1826, hsa-miR-490-3p, hsa-miR-1267, hsa-miR-516a-3p, hsa-miR-520a-3p, hsa-miR-661, hsa-miR-148a*, hsa-miR-548m, hsa-miR-183*, hsa-miR-519c-3p, hsa-miR-194, hsa-miR-548n, hsa-miR-302e, hsa-miR-134, hsa-miR-519a, hsa-miR-1268, hsa-miR-298, hsa-miR-587, hsa-miR-216b, hsa-miR-103, hsa-miR-660, hsa-miR-576-5p, hsa-miR-582-5p, hsa-miR-1296, hsa-miR-34a*, hsa-miR-1236, hsa-miR-618, hsa-miR-10a, hsa-miR-33a*, hsa-miR-302c, hsa-miR-302f, hsa-miR-606, hsa-miR-641, hsa-miR-516b*, hsa-miR-145*, hsa-miR-582-3p, hsa-miR-371-5p, hsa-miR-29a*, hsa-miR-622, hsa-miR-548o, hsa-miR-373, hsa-miR-200c, hsa-miR-145, hsa-miR-616*, hsa-miR-373*, hsa-miR-548f, hsa-miR-573, hsa-miR-1269, hsa-miR-302d, hsa-miR-1202, hsa-miR-495, hsa-miR-513c, hsa-miR-1200, hsa-miR-135a, hsa-miR-520c-3p, hsa-miR-498, hsa-miR-1323, hsa-miR-125b, hsa-miR-302a, hsa-miR-888, hsa-miR-802, hsa-miR-429, hsa-miR-570, hsa-miR-124, hsa-miR-517*, hsa-miR-940, hsa-miR-34b, hsa-miR-1270, hsa-miR-769-3p, hsa-miR-450b-3p, hsa-miR-520d-3p, hsa-miR-450b-5p, hsa-miR-519d, hsa-miR-144*, hsa-miR-135b, hsa-miR-9*, hsa-miR-607, hsa-miR-520e, hsa-miR-578, hsa-miR-93, hsa-miR-566, hsa-miR-548a-3p, hsa-miR-516b, hsa-miR-526b, hsa-miR-620, hsa-miR-223, hsa-miR-526b*, hsa-miR-708*, hsa-miR-628-5p, hsa-miR-1273, hsa-miR-205, hsa-miR-185, hsa-miR-20b, hsa-miR-106a, hsa-miR-877*, hsa-miR-30b*, hsa-miR-758, hsa-miR-10b, hsa-miR-197, hsa-miR-339-5p, hsa-miR-1827, hsa-miR-17, hsa-miR-1279, hsa-miR-297, hsa-miR-548k, hsa-miR-1274b, hsa-miR-548l, hsa-miR-506, hsa-miR-302b, hsa-miR-548e, hsa-miR-485-5p, hsa-miR-377*, hsa-miR-107, hsa-miR-374a, hsa-miR-556-3p, hsa-miR-601, hsa-miR-20a, hsa-miR-425, hsa-miR-340, hsa-miR-140-3p, hsa-miR-106b, hsa-miR-369-3p, hsa-miR-649, hsa-miR-372, hsa-miR-1183, hsa-miR-223*, hsa-miR-433, hsa-miR-154, hsa-miR-135b*.

JTV1—JTV1—NM_{—}006303—hsa-miR-1276, hsa-miR-549, hsa-miR-29c, hsa-miR-29b, hsa-miR-193a-3p, hsa-miR-29a, hsa-miR-935, hsa-miR-514, hsa-miR-193b, hsa-miR-1322, hsa-miR-381, hsa-miR-345, hsa-miR-655, hsa-miR-300, hsa-miR-767-5p.

Isoprenyl-cysteine carboxyl methyltransferase—ICMT—NM_{—}012405—hsa-miR-194*, hsa-miR-510, hsa-miR-34c-5p, hsa-miR-645, hsa-miR-380, hsa-miR-16, hsa-miR-515-5p, hsa-miR-22, hsa-miR-922, hsa-miR-138, hsa-miR-216a, hsa-miR-219-1-3p, hsa-let-7d, hsa-miR-513a-5, hsa-miR-766, hsa-miR-650, hsa-miR-1276, hsa-miR-1184, hsa-miR-9, hsa-miR-101*, hsa-miR-181a, hsa-miR-551b*, hsa-miR-519b-3p, hsa-miR-644, hsa-miR-204, hsa-miR-193 a-5p, hsa-miR-532-3p, hsa-miR-377, hsa-miR-549, hsa-miR-92a-2*, hsa-miR-516a-3p, hsa-miR-496, hsa-miR-661, hsa-miR-99a, hsa-miR-24-1*, hsa-miR-486-3p, hsa-miR-449a, hsa-miR-29b-1*, hsa-miR-330-3p, hsa-miR-28-5p, hsa-miR-1226*, hsa-miR-1237, hsa-miR-519c-3p, hsa-miR-19b, hsa-miR-541*, hsa-miR-217, hsa-miR-146b-3p, hsa-let-7c*, hsa-miR-519a, hsa-miR-1268, hsa-miR-508-5p, hsa-miR-130b, hsa-miR-1231, hsa-let-7e, hsa-miR-1280, hsa-miR-875-3p, hsa-miR-216b, hsa-miR-580, hsa-miR-586, hsa-miR-524-5p, hsa-miR-143*, hsa-miR-186, hsa-miR-23a, hsa-miR-301b, hsa-miR-449b, hsa-miR-33a*, hsa-miR-302f, hsa-miR-1201, hsa-miR-410, hsa-miR-421, hsa-miR-24-2*, hsa-miR-516b*, hsa-miR-646, hsa-miR-708, hsa-miR-1281, hsa-miR-1, hsa-miR-1227, hsa-miR-1207-3p, hsa-miR-130a, hsa-miR-220c, hsa-miR-664, hsa-let-7f, hsa-miR-573, hsa-miR-32*, hsa-miR-15b, hsa-miR-1324, hsa-miR-188-3p, hsa-miR-96*, hsa-miR-502-5p, hsa-miR-495, hsa-miR-513c, hsa-miR-589*, hsa-miR-1291, hsa-miR-1200, hsa-miR-885-3p, hsa-miR-34a, hsa-miR-605, hsa-miR-518a-5p, hsa-miR-608, hsa-miR-886-5p, hsa-miR-1224-5p, hsa-miR-888, hsa-miR-654-3p, hsa-miR-21*, hsa-miR-503, hsa-miR-488, hsa-miR-570, hsa-let-7g, hsa-miR-940, hsa-miR-34b, hsa-miR-1294, hsa-miR-210, hsa-miR-148b*, hsa-miR-1285, hsa-miR-195, hsa-miR-331-5p, hsa-miR-519d, hsa-miR-198, hsa-miR-545, hsa-miR-181d, hsa-miR-612, hsa-miR-9*, hsa-miR-200a, hsa-miR-629*, hsa-miR-93, hsa-miR-1206, hsa-miR-554, hsa-miR-200b*, hsa-miR-518c*, hsa-miR-1260, hsa-let-7i, hsa-miR-23b, hsa-miR-101, hsa-miR-1229, hsa-miR-516b, hsa-miR-526b, hsa-let-7b, hsa-miR-330-5p, hsa-miR-301a, hsa-miR-141, hsa-let-7a, hsa-miR-526b*, hsa-miR-22*, hsa-miR-98, hsa-miR-203, hsa-miR-708*, hsa-miR-744*, hsa-miR-211, hsa-miR-519e*, hsa-miR-361-3p, hsa-miR-628-5p, hsa-miR-150, hsa-miR-181c, hsa-miR-583, hsa-miR-501-5p, hsa-miR-527, hsa-miR-20b, hsa-miR-1256, hsa-miR-640, hsa-miR-106a, hsa-miR-877*, hsa-miR-18a*, hsa-miR-30b*, hsa-miR-99b, hsa-miR-130a*, hsa-miR-483-5p, hsa-miR-522, hsa-let-7c, hsa-miR-202*, hsa-miR-1827, hsa-miR-100, hsa-miR-17, hsa-miR-663b, hsa-miR-106a*, hsa-miR-454, hsa-miR-1274b, hsa-miR-424, hsa-miR-654-5p, hsa-miR-181b, hsa-miR-1283, hsa-miR-485-5p, hsa-miR-614, hsa-miR-137, hsa-miR-331-3p, hsa-miR-888*, hsa-miR-206, hsa-miR-122, hsa-miR-377*, hsa-miR-19a, hsa-miR-188-5p, hsa-miR-15a, hsa-miR-485-3p, hsa-miR-632, hsa-miR-564, hsa-miR-601, hsa-miR-20a, hsa-miR-338-3p, hsa-miR-340, hsa-miR-193b*, hsa-miR-106b, hsa-miR-497, hsa-miR-16-1*, hsa-miR-346, hsa-miR-367*, hsa-miR-326, hsa-miR-497*, hsa-miR-433, hsa-miR-1234, hsa-miR-1275, hsa-miR-541, hsa-miR-520d-5p, hsa-miR-200a*, hsa-miR-1205, hsa-miR-613, hsa-miR-512-5p, hsa-miR-340*.

DNA (cytosine-5-)-methyltransferase 3 alpha—DNMT3A—Variant 1, Variant 2 and Variant 3—NM_{—}175629, NM_{—}153759 and NM_{—}022552—hsa-miR-200b, hsa-miR-130b*, hsa-miR-19b-2*, hsa-miR-548g, hsa-miR-204, hsa-let-7g*, hsa-miR-532-3p, hsa-miR-490-3p, hsa-miR-1248, hsa-miR-920, hsa-miR-92a-2*, hsa-miR-148a*, hsa-miR-143, hsa-miR-129-5p, hsa-miR-943, hsa-miR-194, hsa-miR-19a*, hsa-miR-298, hsa-miR-370, hsa-miR-1280, hsa-miR-1207-5p, hsa-miR-19b-1*, hsa-miR-30b, hsa-miR-548d-3p, hsa-miR-146a*, hsa-miR-30a*, hsa-miR-30a, hsa-miR-29a*, hsa-miR-200c, hsa-miR-581, hsa-miR-362-5p, hsa-miR-720, hsa-miR-188-3p, hsa-miR-599, hsa-miR-495, hsa-miR-29c, hsa-miR-589, hsa-miR-29b, hsa-miR-147, hsa-miR-1233, hsa-miR-30d*, hsa-miR-30e*, hsa-miR-429, hsa-miR-29a, hsa-miR-570, hsa-miR-148b*, hsa-miR-578, hsa-miR-30d, hsa-miR-518c*, hsa-miR-523, hsa-miR-1260, hsa-miR-942, hsa-miR-101, hsa-miR-567, hsa-miR-149*, hsa-miR-211, hsa-miR-150, hsa-miR-30c, hsa-miR-1224-3p, hsa-miR-199a-3p, hsa-miR-149, hsa-miR-30b*, hsa-miR-548c-3p, hsa-miR-297, hsa-miR-626, hsa-miR-199b-3p, hsa-miR-1244, hsa-miR-627, hsa-miR-767-5p, hsa-miR-193b*, hsa-miR-1183, hsa-miR-30e.

DNMT3A—Variant 4—NM_{—}175630—hsa-miR-510, hsa-miR-200b, hsa-miR-1266, hsa-miR-518a-3p, hsa-miR-1306, hsa-miR-518e, hsa-miR-650, hsa-miR-487a, hsa-miR-548g, hsa-miR-30c-1*, hsa-miR-490-3p, hsa-miR-575, hsa-miR-661, hsa-miR-513a-3p, hsa-miR-335*, hsa-miR-217, hsa-miR-146b-3p, hsa-miR-508-5p, hsa-miR-370, hsa-miR-1207-5p, hsa-miR-875-3p, hsa-miR-1252, hsa-miR-630, hsa-miR-30c-2*, hsa-miR-33a*, hsa-miR-302f, hsa-miR-548o, hsa-miR-200c, hsa-miR-145, hsa-miR-518b, hsa-miR-1291, hsa-miR-873, hsa-miR-1323, hsa-miR-429, hsa-miR-105, hsa-miR-1285, hsa-miR-423-3p, hsa-miR-15a*, hsa-miR-612, hsa-miR-328, hsa-miR-551a, hsa-miR-518c, hsa-miR-492, hsa-miR-744*, hsa-miR-361-3p, hsa-miR-1300, hsa-miR-221*, hsa-miR-1321, hsa-miR-18a*, hsa-miR-30b*, hsa-miR-486-5p, hsa-miR-24, hsa-miR-1299, hsa-miR-518f, hsa-miR-7, hsa-miR-182, hsa-miR-345, hsa-miR-654-5p, hsa-miR-1283, hsa-miR-187*, hsa-miR-518d-3p, hsa-miR-485-3p, hsa-miR-183, hsa-miR-551b, hsa-miR-367*, hsa-miR-541, hsa-miR-891b, hsa-miR-558, hsa-miR-154*.

Histamine N-methyltransferase—HNMT—Variant 1 and Variant 2—NM_{—}006985 and NM_{—}001024074—hsa-miR-124*, hsa-miR-557, hsa-miR-32, hsa-miR-542-3p, hsa-miR-222*, hsa-miR-374b, hsa-miR-487a, hsa-miR-221, hsa-miR-127-5p, hsa-miR-516a-3p, hsa-miR-452, hsa-miR-320c, hsa-miR-513a-3p, hsa-miR-142-5p, hsa-miR-335*, hsa-miR-507, hsa-miR-1226*, hsa-miR-129-5p, hsa-miR-943, hsa-miR-1288, hsa-miR-134, hsa-miR-92b, hsa-miR-619, hsa-miR-587, hsa-miR-199a-5p, hsa-miR-623, hsa-miR-1252, hsa-miR-630, hsa-miR-580, hsa-miR-148a, hsa-miR-299-5p, hsa-miR-576-5p, hsa-miR-548p, hsa-miR-586, hsa-miR-367, hsa-miR-524-5p, hsa-miR-543, hsa-miR-34a*, hsa-miR-25, hsa-miR-186, hsa-miR-33a*, hsa-miR-516b*, hsa-let-7b*, hsa-miR-590-5p, hsa-miR-494, hsa-miR-548o, hsa-miR-148b, hsa-miR-362-5p, hsa-miR-664, hsa-miR-1269, hsa-miR-765, hsa-miR-624, hsa-miR-320d, hsa-miR-96*, hsa-miR-590-3p, hsa-miR-7-1*, hsa-miR-1208, hsa-miR-643, hsa-miR-579, hsa-miR-361-5p, hsa-miR-520f, hsa-miR-18b, hsa-miR-1323, hsa-miR-384, hsa-miR-653, hsa-miR-802, hsa-miR-596, hsa-miR-21*, hsa-miR-570, hsa-miR-124, hsa-miR-325, hsa-miR-1261, hsa-miR-222, hsa-miR-768-3p, hsa-miR-125a-3p, hsa-miR-15a*, hsa-miR-20a*, hsa-miR-545, hsa-miR-664*, hsa-miR-323-3p, hsa-miR-200a, hsa-miR-629, hsa-miR-155*, hsa-miR-152, hsa-miR-1255a, hsa-miR-33b, hsa-miR-634, hsa-miR-526b, hsa-miR-139-5p, hsa-miR-141, hsa-miR-22*, hsa-miR-92a, hsa-miR-593*, hsa-let-7f-1*, hsa-miR-1300, hsa-miR-628-5p, hsa-let-7e*, hsa-miR-199b-5p, hsa-miR-493, hsa-miR-18a, hsa-miR-556-5p, hsa-miR-100*, hsa-miR-199a-3p, hsa-miR-758, hsa-miR-320a, hsa-miR-548c-3p, hsa-miR-196a*, hsa-miR-382, hsa-miR-656, hsa-miR-320b, hsa-miR-1264, hsa-miR-548, hsa-miR-506, hsa-miR-21, hsa-miR-199b-3p, hsa-miR-577, hsa-miR-7-2*, hsa-miR-33a, hsa-miR-188-5p, hsa-miR-485-3p, hsa-miR-374a, hsa-miR-455-3p, hsa-miR-126*, hsa-miR-363, hsa-let-7a*, hsa-miR-767-5p, hsa-miR-944, hsa-miR-340, hsa-miR-218, hsa-miR-223*, hsa-miR-602, hsa-miR-1255b, hsa-miR-520d-5p, hsa-miR-154*, hsa-miR-671-5p.

HNMT—Variant 3—NM_{—}001024075—hsa-miR-182*, hsa-miR-330-3p, hsa-miR-1305, hsa-miR-590-3p, hsa-miR-7-1*, hsa-miR-325, hsa-miR-450b-5p, hsa-miR-323-3p, hsa-miR-1206, hsa-miR-656, hsa-miR-892a, hsa-miR-7-2*, hsa-miR-425, hsa-miR-140-3p, hsa-miR-433.

Methyltransferase like 7A—METTL7A—NM_{—}014033—hsa-miR-665, hsa-miR-510, hsa-miR-200b, hsa-miR-520b, hsa-miR-32, hsa-miR-141*, hsa-miR-130b*, hsa-miR-19b-2*, hsa-miR-766, hsa-miR-9, hsa-miR-520g, hsa-miR-616, hsa-miR-195*, hsa-miR-644, hsa-miR-548g, hsa-miR-204, hsa-miR-532-3p, hsa-miR-30c-1*, hsa-miR-549, hsa-miR-335, hsa-miR-138-1*, hsa-miR-363*, hsa-miR-127-5p, hsa-miR-452, hsa-miR-16-2*, hsa-miR-320c, hsa-miR-520a-3p, hsa-miR-661, hsa-miR-513a-3p, hsa-miR-26a-1*, hsa-miR-335*, hsa-miR-302a*, hsa-miR-1237, hsa-miR-548n, hsa-miR-1178, hsa-miR-302e, hsa-miR-19a*, hsa-miR-128, hsa-miR-342-3p, hsa-miR-298, hsa-miR-508-5p, hsa-miR-92b, hsa-miR-587, hsa-miR-660, hsa-miR-548p, hsa-miR-367, hsa-miR-338-5p, hsa-miR-19b-1*, hsa-miR-25, hsa-miR-30c-2*, hsa-miR-10a, hsa-miR-598, hsa-miR-302c, hsa-miR-491-5p, hsa-miR-410, hsa-miR-421, hsa-miR-641, hsa-miR-548d-3p, hsa-miR-411, hsa-miR-548o, hsa-miR-373, hsa-miR-200c, hsa-miR-616*, hsa-miR-373*, hsa-miR-548f, hsa-miR-26b, hsa-miR-32*, hsa-miR-302d, hsa-miR-1202, hsa-miR-628-3p, hsa-miR-320d, hsa-miR-495, hsa-miR-1297, hsa-miR-7-1*, hsa-miR-1253, hsa-miR-643, hsa-miR-26a-2*, hsa-miR-520f, hsa-miR-146a, hsa-miR-376a*, hsa-miR-186*, hsa-miR-520c-3p, hsa-miR-518a-5p, hsa-miR-608, hsa-miR-1323, hsa-miR-214*, hsa-miR-220b, hsa-miR-302a, hsa-miR-21*, hsa-miR-429, hsa-miR-570, hsa-miR-940, hsa-miR-34b, hsa-miR-125a-3p, hsa-miR-1285, hsa-miR-520d-3p, hsa-miR-519d, hsa-miR-545, hsa-miR-504, hsa-miR-612, hsa-miR-9*, hsa-miR-664*, hsa-miR-520e, hsa-miR-10b*, hsa-miR-629*, hsa-miR-93, hsa-miR-155*, hsa-miR-548b-3p, hsa-miR-1274a, hsa-miR-548a-3p, hsa-miR-146b-5p, hsa-miR-567, hsa-miR-302d*, hsa-miR-526b*, hsa-miR-92a, hsa-miR-211, hsa-miR-150, hsa-miR-527, hsa-miR-20b, hsa-miR-1321, hsa-miR-640, hsa-miR-106a, hsa-miR-138-2*, hsa-miR-30b*, hsa-miR-520h, hsa-miR-320a, hsa-miR-10b, hsa-miR-431, hsa-miR-339-5p, hsa-miR-1228, hsa-miR-1827, hsa-miR-548c-3p, hsa-miR-17, hsa-miR-196a*, hsa-miR-320b, hsa-miR-1264, hsa-miR-297, hsa-miR-548k, hsa-miR-675, hsa-miR-548l, hsa-miR-654-5p, hsa-miR-302b, hsa-miR-1283, hsa-miR-548e, hsa-miR-485-5p, hsa-miR-924, hsa-miR-577, hsa-miR-7-2*, hsa-miR-377*, hsa-miR-556-3p, hsa-miR-20a, hsa-miR-126*, hsa-miR-363, hsa-miR-944, hsa-miR-499-5p, hsa-miR-106b, hsa-miR-302b*, hsa-miR-372, hsa-miR-26a, hsa-miR-541, hsa-miR-340*.

Methyltransferase like 2A—METTL2A—NM_{—}181725.3—hsa-miR-502-3p, hsa-miR-520b, hsa-miR-515-5p, hsa-miR-22, hsa-miR-625, hsa-miR-216a, hsa-miR-562, hsa-miR-520g, hsa-miR-214, hsa-miR-548a-5p, hsa-miR-559, hsa-miR-548b-5p, hsa-miR-520a-3p, hsa-miR-513a-3p, hsa-miR-379*, hsa-miR-1288, hsa-miR-302e, hsa-miR-769-5p, hsa-miR-548c-5p, hsa-miR-603, hsa-miR-1825, hsa-miR-1287, hsa-miR-1182, hsa-miR-129*, hsa-miR-299-5p, hsa-miR-524-5p, hsa-miR-143*, hsa-miR-302c, hsa-miR-548i, hsa-miR-30a*, hsa-miR-1227, hsa-miR-373, hsa-miR-616*, hsa-miR-552, hsa-miR-373*, hsa-miR-1269, hsa-miR-302d, hsa-miR-1200, hsa-miR-411*, hsa-miR-186*, hsa-miR-520c-3p, hsa-miR-498, hsa-miR-384, hsa-miR-302a, hsa-miR-30d*, hsa-miR-30e*, hsa-miR-21*, hsa-miR-548d-5p, hsa-miR-588, hsa-miR-768-3p, hsa-miR-512-3p, hsa-miR-520d-3p, hsa-miR-519d, hsa-miR-548j, hsa-miR-520e, hsa-miR-93, hsa-miR-941, hsa-miR-526b*, hsa-miR-548h, hsa-miR-744*, hsa-miR-519e*, hsa-miR-150, hsa-miR-20b, hsa-miR-378*, hsa-miR-556-5p, hsa-miR-106a, hsa-miR-483-5p, hsa-miR-550, hsa-miR-520h, hsa-miR-431, hsa-miR-129-3p, hsa-miR-17, hsa-miR-297, hsa-miR-302b, hsa-miR-187*, hsa-miR-924, hsa-miR-20a, hsa-miR-876-3p, hsa-miR-126*, hsa-miR-501-3p, hsa-miR-193b*, hsa-miR-219-2-3p, hsa-miR-106b, hsa-miR-372, hsa-miR-367*, hsa-miR-520d-5p, hsa-miR-558, hsa-miR-135b*, hsa-miR-512-5p.

Methyltransferase like 2B—METTL2B—NM_{—}018396—hsa-miR-502-3p, hsa-miR-520b, hsa-miR-216a, hsa-miR-520g, hsa-miR-214, hsa-miR-548a-5p, hsa-miR-559, hsa-miR-548b-5p, hsa-miR-520a-3p, hsa-miR-513a-3p, hsa-miR-378, hsa-miR-302e, hsa-miR-769-5p, hsa-miR-548c-5p, hsa-miR-1287, hsa-miR-1182, hsa-miR-129*, hsa-miR-299-5p, hsa-miR-524-5p, hsa-miR-302c, hsa-miR-548i, hsa-miR-410, hsa-miR-421, hsa-miR-329, hsa-miR-30a*, hsa-miR-373, hsa-miR-616*, hsa-miR-552, hsa-miR-373*, hsa-miR-99a*, hsa-miR-302d, hsa-miR-1200, hsa-miR-186*, hsa-miR-520c-3p, hsa-miR-384, hsa-miR-302a, hsa-miR-30d*, hsa-miR-30e*, hsa-miR-21*, hsa-miR-548d-5p, hsa-miR-588, hsa-miR-125a-3p, hsa-miR-512-3p, hsa-miR-520d-3p, hsa-miR-519d, hsa-miR-548j, hsa-miR-520e, hsa-miR-578, hsa-miR-10b*, hsa-miR-93, hsa-miR-422a, hsa-miR-362-3p, hsa-miR-526b*, hsa-miR-548h, hsa-miR-744*, hsa-miR-150, hsa-miR-20b, hsa-miR-378*, hsa-miR-106a, hsa-miR-483-5p, hsa-miR-550, hsa-miR-520h, hsa-miR-522, hsa-miR-431, hsa-miR-129-3p, hsa-miR-17, hsa-miR-297, hsa-miR-99b*, hsa-miR-302b, hsa-miR-626, hsa-miR-1283, hsa-miR-187*, hsa-miR-924, hsa-miR-20a, hsa-miR-876-3p, hsa-miR-338-3p, hsa-miR-767-5p, hsa-miR-501-3p, hsa-miR-193b*, hsa-miR-219-2-3p, hsa-miR-106b, hsa-miR-372, hsa-miR-1250, hsa-miR-520d-5p.

Ezrin—EZR—NM_{—}003379—hsa-miR-376b, hsa-miR-509-3-5p, hsa-miR-380, hsa-miR-22, hsa-miR-138, hsa-miR-1228*, hsa-miR-648, hsa-miR-1184, hsa-miR-133a, hsa-miR-551b*, hsa-miR-548g, hsa-miR-204, hsa-miR-96, hsa-miR-214, hsa-miR-377, hsa-miR-218-1*, hsa-miR-890, hsa-miR-335*, hsa-miR-548m, hsa-miR-129-5p, hsa-miR-885-5p, hsa-miR-134, hsa-miR-663, hsa-miR-1252, hsa-miR-630, hsa-miR-148a, hsa-miR-548p, hsa-miR-524-5p, hsa-miR-33a*, hsa-let-7b*, hsa-miR-153, hsa-miR-1281, hsa-miR-548o, hsa-miR-376a, hsa-miR-1207-3p, hsa-miR-148b, hsa-miR-548f, hsa-miR-1202, hsa-miR-765, hsa-miR-495, hsa-miR-1271, hsa-miR-589*, hsa-miR-885-3p, hsa-miR-643, hsa-miR-520f, hsa-miR-518a-5p, hsa-miR-185*, hsa-miR-1323, hsa-miR-384, hsa-miR-654-3p, hsa-miR-568, hsa-miR-596, hsa-miR-570, hsa-miR-940, hsa-miR-34b, hsa-miR-432, hsa-miR-769-3p, hsa-miR-450b-3p, hsa-miR-15a*, hsa-miR-450b-5p, hsa-miR-566, hsa-miR-152, hsa-miR-1322, hsa-miR-548a-3p, hsa-miR-133b, hsa-miR-211, hsa-let-7f-1*, hsa-miR-628-5p, hsa-miR-205, hsa-miR-527, hsa-miR-18a*, hsa-miR-548c-3p, hsa-miR-345, hsa-miR-548e, hsa-miR-892a, hsa-miR-632, hsa-miR-183, hsa-let-7a*, hsa-miR-338-3p, hsa-miR-509-5p, hsa-let-7f-2*, hsa-miR-595, hsa-miR-499-3p, hsa-miR-520d-5p, hsa-miR-744.

Tumor protein D52—TPD52—Variant 1, Variant 2 and Variant 3—NM_{—}001025252, NM_{—}001025253 and NM_{—}005079—hsa-miR-576-3p, hsa-miR-194*, hsa-miR-124*, hsa-miR-376b, hsa-miR-665, hsa-miR-34c-5p, hsa-miR-448, hsa-miR-648, hsa-miR-219-1-3p, hsa-miR-374b, hsa-miR-101*, hsa-miR-181a, hsa-miR-182*, hsa-miR-195*, hsa-miR-302c*, hsa-miR-483-3p, hsa-miR-204, hsa-miR-668, hsa-miR-218-1*, hsa-miR-490-3p, hsa-miR-452, hsa-miR-16-2*, hsa-miR-26a-1*, hsa-miR-486-3p, hsa-miR-335*, hsa-miR-449a, hsa-miR-548m, hsa-miR-1237, hsa-let-7c*, miR-129*, hsa-miR-1246, hsa-miR-299-5p, hsa-miR-586, hsa-miR-582-5p, hsa-miR-338-5p, hsa-miR-186, hsa-miR-449b, hsa-miR-1236, hsa-miR-618, hsa-miR-33a*, hsa-miR-218-2*, hsa-miR-410, hsa-miR-421, hsa-miR-548d-3p, hsa-let-7b*, hsa-miR-151-3p, hsa-miR-145*, hsa-miR-411, hsa-miR-622, hsa-miR-548o, hsa-miR-376a, hsa-miR-145, hsa-miR-220c, hsa-miR-532-5p, hsa-miR-188-3p, hsa-miR-590-3p, hsa-miR-495, hsa-miR-513c, hsa-miR-7-1*, hsa-miR-767-3p, hsa-miR-589*, hsa-miR-34a, hsa-miR-936, hsa-miR-26a-2*, hsa-miR-146a, hsa-miR-768-5p, hsa-miR-873, hsa-miR-144, hsa-miR-1323, hsa-miR-653, hsa-miR-553, hsa-miR-570, hsa-miR-325, hsa-miR-432, hsa-miR-34b*, hsa-miR-15a*, hsa-miR-452*, hsa-miR-181d, hsa-miR-9*, hsa-miR-636, hsa-miR-548b-3p, hsa-miR-1257, hsa-miR-609, hsa-miR-146b-5p, hsa-miR-516b, hsa-miR-136, hsa-miR-139-5p, hsa-miR-203, hsa-miR-571, hsa-miR-211, hsa-let-7f-1*, hsa-miR-181c, hsa-miR-181a-2*, hsa-miR-583, hsa-miR-205, hsa-miR-224, hsa-miR-1284, hsa-miR-221*, hsa-miR-185, hsa-miR-100*, hsa-miR-1224-3p, hsa-miR-138-2*, hsa-miR-522, hsa-miR-129-3p, hsa-miR-663b, hsa-miR-1279, hsa-miR-656, hsa-miR-548k, hsa-miR-181b, hsa-miR-1251, hsa-miR-7-2*, hsa-miR-107, hsa-miR-374a, hsa-miR-1244, hsa-let-7a*, hsa-miR-1272, hsa-let-7f-2*, hsa-miR-218, hsa-miR-142-3p, hsa-miR-1250, hsa-miR-512-5p.

Actin related protein 2/3 complex, subunit 5 16 kDa—ARPC5—NM_{—}005717—hsa-miR-557, hsa-miR-642, hsa-miR-380, hsa-miR-22, hsa-miR-562, hsa-miR-19b-2*, hsa-miR-875-5p, hsa-miR-650, hsa-miR-644, hsa-miR-549, hsa-miR-548a-5p, hsa-miR-559, hsa-miR-548b-5p, hsa-miR-320c, hsa-miR-544, hsa-miR-335*, hsa-miR-507, hsa-miR-190, hsa-miR-302a*, hsa-miR-561, hsa-miR-129-5p, hsa-miR-548c-5p, hsa-miR-19a*, hsa-miR-216b, hsa-miR-548p, hsa-miR-19b-1*, hsa-miR-30b, hsa-miR-548i, hsa-miR-641, hsa-miR-548d-3p, hsa-miR-146a*, hsa-miR-30a, hsa-miR-29a*, hsa-miR-548o, hsa-miR-145, hsa-miR-548f, hsa-miR-220c, hsa-miR-320d, hsa-miR-589*, hsa-miR-190b, hsa-miR-885-3p, hsa-miR-193a-3p, hsa-miR-146a, hsa-miR-374b*, hsa-miR-147, hsa-miR-509-3p, hsa-miR-1262, hsa-miR-1323, hsa-miR-568, hsa-miR-488, hsa-miR-570, hsa-miR-548d-5p, hsa-miR-450b-5p, hsa-miR-610, hsa-miR-545, hsa-miR-548j, hsa-miR-200a, hsa-miR-30d, hsa-miR-193b, hsa-miR-1206, hsa-miR-101, hsa-miR-548a-3p, hsa-miR-146b-5p, hsa-miR-634, hsa-miR-141, hsa-miR-203, hsa-miR-548h, hsa-miR-30c, hsa-miR-378*, hsa-miR-556-5p, hsa-miR-320a, hsa-miR-522, hsa-miR-1827, hsa-miR-106a*, hsa-miR-656, hsa-miR-320b, hsa-miR-548e, hsa-miR-655, hsa-miR-485-5p, hsa-miR-888*, hsa-miR-876-3p, hsa-miR-423-5p, hsa-miR-340, hsa-miR-369-3p, hsa-let-7f-2*, hsa-miR-16-1*, hsa-miR-223*, hsa-miR-499-3p, hsa-miR-30e.

Nucleolar protein 8—NOL8—NM_{—}017948—hsa-miR-510, hsa-miR-520b, hsa-miR-645, hsa-miR-181a, hsa-miR-548g, hsa-miR-377, hsa-miR-452, hsa-miR-520a-3p, hsa-miR-513a-3p, hsa-miR-548n, hsa-miR-302e, hsa-miR-875-3p, hsa-miR-374a*, hsa-miR-23a, hsa-miR-302c, hsa-let-7b*, hsa-miR-146a*, hsa-miR-373, hsa-miR-548f, hsa-miR-302d, hsa-miR-1253, hsa-miR-520f, hsa-miR-520c-3p, hsa-miR-302a, hsa-miR-520d-3p, hsa-miR-181d, hsa-miR-607, hsa-miR-520e, hsa-miR-23b, hsa-miR-548a-3p, hsa-miR-223, hsa-miR-22*, hsa-miR-381, hsa-let-7f-1*, hsa-miR-1300, hsa-miR-181c, hsa-miR-130a*, hsa-miR-548l, hsa-miR-302b, hsa-miR-181b, hsa-miR-548e, hsa-miR-300, hsa-miR-409-3p, hsa-let-7a*, hsa-miR-372, hsa-miR-223*.

N-ehylmaleimide-sensitive factor—NSF—N_{—}006178—hsa-miR-124*, hsa-miR-877, hsa-miR-32, hsa-miR-16, hsa-miR-513a-5p, hsa-miR-192, hsa-miR-125b-2*, hsa-miR-181a, hsa-miR-195*, hsa-miR-28-3p, hsa-miR-1179, hsa-miR-30c-1*, hsa-miR-218-1*, hsa-miR-890, hsa-miR-516a-3p, hsa-miR-16-2*, hsa-miR-31, hsa-miR-143, hsa-miR-548n, hsa-miR-769-5p, hsa-miR-1308, hsa-miR-92b, hsa-miR-587, hsa-miR-1303, hsa-miR-623, hsa-miR-103, hsa-miR-1252, hsa-miR-651, hsa-miR-576-5p, hsa-miR-367, hsa-miR-582-5p, hsa-miR-338-5p, hsa-miR-543, hsa-miR-25, hsa-miR-30c-2*, hsa-miR-302f, hsa-miR-218-2*, hsa-miR-641, hsa-miR-516b*, hsa-let-7b*, hsa-miR-489, hsa-miR-1227, hsa-miR-616*, hsa-miR-373*, hsa-miR-26b, hsa-miR-628-3p, hsa-miR-532-5p, hsa-miR-15b, hsa-miR-502-5p, hsa-miR-1297, hsa-miR-215, hsa-miR-135a, hsa-miR-579, hsa-miR-193a-3p, hsa-miR-1243, hsa-miR-147, hsa-miR-653, hsa-miR-505*, hsa-miR-1224-5p, hsa-miR-596, hsa-miR-570, hsa-miR-34b, hsa-miR-325, hsa-miR-1294, hsa-miR-588, hsa-miR-432, hsa-miR-1270, hsa-miR-768-3p, hsa-miR-512-3p, hsa-miR-195, hsa-miR-15a*, hsa-miR-452*, hsa-miR-181d, hsa-miR-135b, hsa-miR-514, hsa-miR-607, hsa-miR-193b, hsa-miR-1260, hsa-miR-412, hsa-miR-516b, hsa-miR-526b, hsa-miR-620, hsa-miR-302d*, hsa-miR-223, hsa-miR-571, hsa-miR-92a, hsa-let-7f-1*, hsa-miR-432*, hsa-miR-181c, hsa-miR-583, hsa-miR-205, hsa-miR-224, hsa-miR-185, hsa-miR-1228, hsa-miR-656, hsa-miR-548l, hsa-miR-424, hsa-miR-181b, hsa-miR-1278, hsa-miR-892a, hsa-miR-377*, hsa-miR-107, hsa-miR-15a, hsa-miR-425, hsa-miR-363, hsa-let-7a*, hsa-miR-140-3p, hsa-miR-497, hsa-miR-346, hsa-miR-302b*, hsa-miR-595, hsa-miR-26a, hsa-miR-142-3p, hsa-miR-891b, hsa-miR-95.

RAD23 homolog B—RAD23B—NM_{—}002874—hsa-miR-510, hsa-miR-877, hsa-miR-520b, hsa-miR-34c-5p, hsa-miR-16, hsa-miR-562, hsa-miR-875-5p, hsa-miR-520g, hsa-miR-181a, hsa-miR-195*, hsa-miR-302c*, hsa-miR-204, hsa-let-7g*, hsa-miR-532-3p, hsa-miR-516a-3p, hsa-miR-452, hsa-miR-16-2*, hsa-miR-575, hsa-miR-496, hsa-miR-320c, hsa-miR-520a-3p, hsa-miR-513a-3p, hsa-miR-143, hsa-miR-335*, hsa-miR-449a, hsa-miR-1237, hsa-miR-19b, hsa-miR-146b-3p, hsa-miR-302e, hsa-miR-93*, hsa-miR-603, hsa-miR-199a-5p, hsa-miR-1287, hsa-miR-1182, hsa-miR-548p, hsa-miR-524-5p, hsa-miR-30b, hsa-miR-34a*, hsa-miR-186, hsa-miR-23a, hsa-miR-449b, hsa-miR-302c, hsa-miR-410, hsa-miR-421, hsa-miR-516b*, hsa-miR-494, hsa-miR-411, hsa-miR-30a, hsa-miR-29a*, hsa-miR-324-3p, hsa-miR-548o, hsa-miR-373, hsa-miR-145, hsa-miR-874, hsa-miR-548f, hsa-miR-573, hsa-miR-302d, hsa-miR-624, hsa-miR-15b, hsa-miR-320d, hsa-miR-188-3p, hsa-miR-96*, hsa-miR-590-3p, hsa-miR-1200, hsa-miR-34a, hsa-miR-361-5p, hsa-miR-1259, hsa-miR-186*, hsa-miR-147, hsa-miR-520c-3p, hsa-miR-770-5p, hsa-miR-498, hsa-miR-144, hsa-miR-1323, hsa-miR-214*, hsa-miR-302a, hsa-miR-570, hsa-miR-508-3p, hsa-miR-124, hsa-miR-517*, hsa-miR-512-3p, hsa-miR-520d-3p, hsa-miR-195, hsa-miR-519d, hsa-miR-20a*, hsa-miR-545, hsa-miR-181d, hsa-miR-664*, hsa-miR-520e, hsa-miR-200a, hsa-miR-30d, hsa-miR-629*, hsa-miR-93, hsa-miR-383, hsa-miR-192*, hsa-miR-491-3p, hsa-miR-1260, hsa-miR-23b, hsa-miR-539, hsa-miR-1257, hsa-miR-548a-3p, hsa-miR-136, hsa-miR-141, hsa-miR-526b*, hsa-miR-744*, hsa-miR-211, hsa-miR-150, hsa-miR-30c, hsa-miR-181c, hsa-miR-199b-5p, hsa-miR-20b, hsa-miR-640, hsa-miR-1224-3p, hsa-miR-106a, hsa-miR-1245, hsa-miR-138-2*, hsa-miR-18a*, hsa-miR-130a*, hsa-miR-483-5p, hsa-miR-520h, hsa-miR-320a, hsa-miR-548c-3p, hsa-miR-17, hsa-miR-106a*, hsa-miR-656, hsa-miR-320b, hsa-miR-548k, hsa-miR-424, hsa-miR-506, hsa-miR-302b, hsa-miR-626, hsa-miR-181b, hsa-miR-1283, hsa-miR-187*, hsa-miR-548e, hsa-miR-655, hsa-miR-485-5p, hsa-miR-892a, hsa-miR-577, hsa-miR-19a, hsa-miR-15a, hsa-miR-20a, hsa-miR-944, hsa-miR-340, hsa-miR-193b*, hsa-miR-140-3p, hsa-miR-106b, hsa-miR-497, hsa-miR-1197, hsa-miR-372, hsa-miR-595, hsa-miR-154, hsa-miR-520d-5p, hsa-miR-30e.

Signal recognition particle 54 kDa—SRP54—NM_{—}003136—hsa-miR-452 hsa-miR-1252, hsa-miR-606, hsa-miR-548d-3p, hsa-miR-494, hsa-miR-590-3p, hsa-miR-1253, hsa-miR-570, hsa-miR-105, hsa-miR-331-5p, hsa-miR-545, hsa-miR-629*, hsa-miR-223, hsa-miR-1321, hsa-miR-106a*, hsa-miR-433.

Heat shock 70 kDa protein 2—HSPA2—NM_{—}021979—hsa-miR-448, hsa-miR-302c*, hsa-miR-96, hsa-miR-452, hsa-miR-19b, hsa-miR-548n, hsa-miR-130b, hsa-miR-299-5p, hsa-miR-586, hsa-miR-301b, hsa-miR-337-3p, hsa-miR-33a*, hsa-miR-371-5p, hsa-miR-130a, hsa-miR-552, hsa-miR-617, hsa-miR-495, hsa-miR-1271, hsa-miR-7-1*, hsa-miR-589*, hsa-miR-579, hsa-miR-450b-5p, hsa-miR-545, hsa-miR-323-3p, hsa-miR-491-3p, hsa-miR-301a, hsa-miR-583, hsa-miR-500*, hsa-miR-106a*, hsa-miR-382, hsa-miR-454, hsa-miR-548, hsa-miR-7-2*, hsa-miR-19a.

Mediator complex subunit 1—MED1—NM_{—}004774—hsa-miR-376b, hsa-miR-200b, hsa-miR-877, hsa-miR-1266, hsa-miR-642, hsa-miR-16, hsa-miR-515-5p, hsa-miR-141*, hsa-miR-922, hsa-miR-493*, hsa-miR-875-5p, hsa-miR-1290, hsa-miR-513b, hsa-miR-221, hsa-miR-483-3p, hsa-miR-548g, hsa-miR-204, hsa-miR-96, hsa-miR-604, hsa-miR-668, hsa-miR-1179, hsa-miR-532-3p, hsa-miR-549, hsa-miR-548a-5p, hsa-miR-490-3p, hsa-miR-647, hsa-miR-559, hsa-miR-1248, hsa-miR-920, hsa-miR-548b-5p, hsa-miR-452, hsa-miR-320c, hsa-miR-486-3p, hsa-miR-335*, hsa-miR-29b-1*, hsa-miR-561, hsa-miR-541*, hsa-miR-1178, hsa-miR-217, hsa-miR-892b, hsa-miR-146b-3p, hsa-miR-885-5p, hsa-miR-548c-5p, hsa-miR-128, hsa-miR-298, hsa-miR-587, hsa-miR-1207-5p, hsa-miR-657, hsa-miR-1252, hsa-miR-1182, hsa-miR-651, hsa-miR-580, hsa-miR-129*, hsa-miR-148a, hsa-miR-576-5p, hsa-miR-876-5p, hsa-miR-618, hsa-miR-10a, hsa-miR-33a*, hsa-miR-548i, hsa-miR-641, hsa-miR-212, hsa-miR-548d-3p, hsa-miR-146a*, hsa-miR-371-5p, hsa-miR-489, hsa-miR-29a*, hsa-miR-324-3p, hsa-miR-548o, hsa-miR-1, hsa-miR-376a, hsa-miR-200c, hsa-miR-616*, hsa-miR-148b, hsa-miR-373*, hsa-miR-548f, hsa-miR-765, hsa-miR-532-5p, hsa-miR-15b, hsa-miR-320d, hsa-miR-188-3p, hsa-miR-617, hsa-miR-590-3p, hsa-miR-502-5p, hsa-miR-495, hsa-miR-1271, hsa-miR-132, hsa-miR-591, hsa-miR-7-1*, hsa-miR-939, hsa-miR-635, hsa-miR-589*, hsa-miR-1291, hsa-miR-1253, hsa-miR-579, hsa-miR-146a, hsa-miR-605, hsa-miR-1259, hsa-miR-186*, hsa-miR-873, hsa-miR-27a*, hsa-miR-1226, hsa-miR-144, hsa-miR-1323, hsa-miR-220b, hsa-miR-888, hsa-miR-654-3p, hsa-miR-568, hsa-miR-429, hsa-miR-488, hsa-miR-570, hsa-miR-105, hsa-miR-940, hsa-miR-548d-5p, hsa-miR-222, hsa-miR-195, hsa-miR-9*, hsa-miR-548j, hsa-miR-323-3p, hsa-miR-200a, hsa-miR-629*, hsa-miR-629, hsa-miR-29b-2*, hsa-miR-155*, hsa-miR-548b-3p, hsa-miR-1260, hsa-miR-152, hsa-miR-1274a, hsa-miR-539, hsa-miR-548a-3p, hsa-miR-146b-5p, hsa-miR-634, hsa-miR-328, hsa-miR-139-5p, hsa-miR-141, hsa-miR-548h, hsa-miR-597, hsa-miR-211, hsa-miR-519e*, hsa-miR-181a*, hsa-miR-361-3p, hsa-miR-628-5p, hsa-miR-760, hsa-miR-205, hsa-miR-224, hsa-miR-1321, hsa-miR-138-2*, hsa-miR-877*, hsa-miR-149, hsa-miR-320a, hsa-miR-10b, hsa-miR-202*, hsa-miR-129-3p, hsa-miR-548c-3p, hsa-miR-182, hsa-miR-320b, hsa-miR-345, hsa-miR-1274b, hsa-miR-548l, hsa-miR-424, hsa-miR-548e, hsa-miR-485-5p, hsa-miR-137, hsa-miR-888*, hsa-miR-577, hsa-miR-206, hsa-miR-7-2*, hsa-miR-15a, hsa-miR-556-3p, hsa-miR-183, hsa-miR-876-3p, hsa-miR-1272, hsa-miR-497, hsa-miR-16-1*, hsa-miR-367*, hsa-miR-497*, hsa-miR-433, hsa-miR-154, hsa-miR-499-3p, hsa-miR-1275, hsa-miR-1302, hsa-miR-425*, hsa-miR-613.

THAP domain containing, apoptosis associated protein 2—THAP2—N_{—}031435—hsa-miR-140-5p, hsa-miR-124*, hsa-miR-517a, hsa-miR-200b, hsa-miR-520b, hsa-miR-380, hsa-miR-16, hsa-miR-141*, hsa-miR-922, hsa-miR-562, hsa-miR-875-5p, hsa-miR-650, hsa-miR-9, hsa-miR-520g, hsa-miR-150*, hsa-miR-101*, hsa-miR-181a, hsa-miR-513b, hsa-miR-195*, hsa-miR-96, hsa-miR-28-3p, hsa-miR-1304, hsa-miR-890, hsa-miR-490-3p, hsa-miR-27a, hsa-miR-16-2*, hsa-miR-320c, hsa-miR-520a-3p, hsa-miR-513a-3p, hsa-miR-99a, hsa-miR-142-5p, hsa-miR-335*, hsa-miR-183*, hsa-miR-1226*, hsa-miR-561, hsa-miR-146b-3p, hsa-miR-302e, hsa-miR-1825, hsa-miR-128, hsa-miR-508-5p, hsa-miR-587, hsa-miR-1303, hsa-miR-1287, hsa-miR-103, hsa-let-7d*, hsa-miR-630, hsa-miR-379, hsa-miR-576-5p, hsa-miR-548p, hsa-miR-586, hsa-miR-876-5p, hsa-miR-582-5p, hsa-miR-338-5p, hsa-miR-524-5p, hsa-miR-143*, hsa-miR-543, hsa-miR-374a*, hsa-miR-186, hsa-miR-23a, hsa-miR-365, hsa-miR-1305, hsa-miR-337-3p, hsa-miR-33a*, hsa-miR-302c, hsa-miR-202, hsa-miR-548d-3p, hsa-miR-146a*, hsa-miR-411, hsa-miR-30a*, hsa-miR-373, hsa-miR-200c, hsa-miR-874, hsa-miR-26b, hsa-miR-302d, hsa-miR-15b, hsa-miR-320d, hsa-miR-590-3p, hsa-miR-1271, hsa-miR-29c, hsa-miR-1297, hsa-miR-7-1*, hsa-miR-26b*, hsa-miR-1253, hsa-miR-936, hsa-miR-29b, hsa-miR-361-5p, hsa-miR-520f, hsa-miR-520c-3p, hsa-miR-518a-5p, hsa-miR-653, hsa-miR-505*, hsa-miR-302a, hsa-miR-30d*, hsa-miR-30e*, hsa-miR-21*, hsa-miR-429, hsa-miR-29a, hsa-miR-624*, hsa-miR-570, hsa-miR-105, hsa-miR-124, hsa-MiR-768-3p, hsa-miR-148b*, hsa-miR-512-3p, hsa-miR-520d-3p, hsa-miR-195, hsa-miR-15a*, hsa-miR-450b-5p, hsa-miR-519d, hsa-miR-181d, hsa-miR-9*, hsa-miR-27b, hsa-miR-607, hsa-miR-520e, hsa-miR-629, hsa-miR-93, hsa-miR-192*, hsa-miR-934, hsa-miR-491-3p, hsa-miR-1260, hsa-miR-942, hsa-miR-23b, hsa-miR-23a*, hsa-miR-545*, hsa-miR-539, hsa-miR-1257, hsa-miR-23b*, hsa-miR-567, hsa-miR-136, hsa-miR-139-5p, hsa-miR-889, hsa-miR-526b*, hsa-miR-625*, hsa-miR-150, hsa-miR-181c, hsa-miR-527, hsa-miR-20b, hsa-miR-100*, hsa-miR-1224-3p, hsa-miR-199a-3p, hsa-miR-106a, hsa-miR-138-2*, hsa-miR-30b*, hsa-miR-99b, hsa-miR-130a*, hsa-miR-758, hsa-miR-520h, hsa-miR-320a, hsa-miR-486-5p, hsa-miR-548c-3p, hsa-miR-135a*, hsa-miR-100, hsa-miR-17, hsa-miR-1279, hsa-miR-656, hsa-miR-182, hsa-miR-320b, hsa-miR-1264, hsa-miR-297, hsa-miR-548k, hsa-miR-424, hsa-miR-506, hsa-miR-302b, hsa-miR-181b, hsa-miR-1283, hsa-miR-655, hsa-miR-199b-3p, hsa-miR-577, hsa-miR-7-2*, hsa-miR-377*, hsa-miR-107, hsa-miR-15a, hsa-miR-1244, hsa-miR-20a, hsa-miR-409-3p, hsa-miR-1272, hsa-miR-944, hsa-miR-340, hsa-miR-1265, hsa-miR-219-2-3p, hsa-miR-106b, hsa-miR-497, hsa-miR-369-3p, hsa-miR-517c, hsa-miR-372, hsa-miR-1183, hsa-miR-497*, hsa-miR-595, hsa-miR-26a, hsa-miR-520d-5p, hsa-miR-891b, hsa-miR-584.

Cold inducible RNA binding protein—CIRBP—NN_{—}001280—hsa-miR-1266, hsa-miR-1276, hsa-miR-615-5p, hsa-miR-532-3p, hsa-miR-548a-5p, hsa-miR-559, hsa-miR-27a, hsa-miR-548b-5p, hsa-miR-548n, hsa-miR-548c-5p, hsa-miR-338-5p, hsa-miR-33a*, hsa-miR-548i, hsa-miR-548d-3p, hsa-miR-616*, hsa-miR-373*, hsa-miR-548f, hsa-miR-628-3p, hsa-miR-188-3p, hsa-miR-185*, hsa-miR-548d-5p, hsa-miR-15a*, hsa-miR-27b, hsa-miR-548j, hsa-miR-383, hsa-miR-934, hsa-miR-548b-3p, hsa-miR-545*, hsa-miR-548a-3p, hsa-miR-526b, hsa-miR-571, hsa-miR-548h, hsa-miR-381, hsa-miR-548e, hsa-miR-300.

Small nuclear ribonucleoprotein polypeptide N—SNRPN—Variant 1, Variant 2, Variant 3, Variant 4 and Variant 5—NM_{—}003097, NM_{—}022805, NM_{—}022806, N_{—}022807 and NM_{—}022808—hsa-miR-513b, hsa-miR-134, hsa-miR-586, hsa-miR-302f, hsa-miR-144, hsa-miR-126*.

Superkiller viralicidic activity 2-like 2—SKIV2L2—N_{—}015360—hsa-miR-140-5p, hsa-miR-557, hsa-miR-380, hsa-miR-493*, hsa-miR-216a, hsa-miR-374b, hsa-miR-214, hsa-miR-30c-1*, hsa-miR-1826, hsa-miR-1267, hsa-miR-127-5p, hsa-miR-31*, hsa-miR-31, hsa-miR-507, hsa-miR-330-3p, hsa-miR-651, hsa-miR-876-5p, hsa-miR-23a, hsa-miR-30c-2*, hsa-miR-552, hsa-miR-589, hsa-miR-34c-3p, hsa-miR-605, hsa-miR-1226, hsa-miR-323-5p, hsa-miR-568, hsa-miR-21*, hsa-miR-488, hsa-miR-155, hsa-miR-34b, hsa-miR-125a-3p, hsa-miR-23b, hsa-miR-302d*, hsa-miR-583, hsa-miR-501-5p, hsa-miR-1224-3p, hsa-miR-130a*, hsa-miR-548c-3p, hsa-miR-182, hsa-miR-297, hsa-miR-548l, hsa-miR-655, hsa-miR-485-3p, hsa-miR-374a, hsa-miR-369-3p, hsa-miR-16-1*, hsa-miR-302b*, hsa-miR-1183.

Dual specificity phosphatase 10—DUSP10—Variant 1, Variant 2 and Variant 3—NM_{—}007207, NM_{—}144728 and NM₁₃ 144729—hsa-miR-557, hsa-miR-32, hsa-miR-16, hsa-miR-500, hsa-miR-515-5p, hsa-miR-22, hsa-miR-216a, hsa-miR-551b*, hsa-miR-575, hsa-miR-511, hsa-miR-142-5p, hsa-miR-379*, hsa-miR-507, hsa-miR-548m, hsa-miR-302a*, hsa-miR-129-5p, hsa-miR-541*, hsa-miR-92b, hsa-miR-216b, hsa-miR-1252, hsa-miR-367, hsa-miR-25, hsa-miR-186, hsa-miR-618, hsa-miR-520a-5p, hsa-miR-548d-3p, hsa-miR-329, hsa-miR-525-5p, hsa-miR-874, hsa-miR-1202, hsa-miR-450a, hsa-miR-624, hsa-miR-15b, hsa-miR-411*, hsa-miR-147, hsa-miR-653, hsa-miR-195, hsa-miR-92a-1*, hsa-miR-942, hsa-miR-362-3p, hsa-miR-302d*, hsa-miR-223, hsa-miR-92 a, hsa-miR-519e*, hsa-miR-150, hsa-miR-501-5p, hsa-miR-659, hsa-miR-138-2*, hsa-miR-18a*, hsa-miR-30b*, hsa-miR-548c-3p, hsa-miR-382, hsa-miR-656, hsa-miR-548l, hsa-miR-424, hsa-miR-1283, hsa-miR-15a, hsa-miR-183, hsa-miR-363, hsa-miR-497, hsa-miR-302b*, hsa-miR-595.

Signal sequence receptor, alpha—SSR1—NM_{—}003144—hsa-miR-576-3p, hsa-miR-194*, hsa-miR-376b, hsa-miR-502-3p, hsa-miR-665, hsa-miR-510, hsa-miR-200b, hsa-miR-509-3-5p, hsa-miR-520b, hsa-miR-1266, hsa-miR-34c-5p, hsa-miR-1254, hsa-miR-645, hsa-miR-16, hsa-miR-500, hsa-miR-27b*, hsa-miR-141*, hsa-miR-22, hsa-miR-922, hsa-miR-625, hsa-miR-138, hsa-miR-648, hsa-miR-562, hsa-miR-19b-2*, hsa-miR-513a-5p, hsa-miR-766, hsa-miR-650, hsa-miR-192, hsa-miR-1276, hsa-miR-1184, hsa-miR-125b-2*, hsa-miR-520g, hsa-miR-1290, hsa-miR-1301, hsa-miR-181a, hsa-miR-551b*, hsa-miR-182*, hsa-miR-195*, hsa-miR-302c*, hsa-miR-125a-5p, hsa-miR-487a, hsa-miR-548g, hsa-miR-214, hsa-miR-377, hsa-miR-218-1*, hsa-miR-1304, hsa-miR-890, hsa-miR-376c, hsa-miR-490-3p, hsa-miR-27a, hsa-miR-1248, hsa-miR-335, hsa-miR-138-1*, hsa-miR-363*, hsa-miR-127-5p, hsa-miR-16-2*, hsa-miR-496, hsa-miR-320c, hsa-miR-520a-3p, hsa-miR-511, hsa-miR-661, hsa-miR-15b*, hsa-miR-31, hsa-miR-513a-3p, hsa-miR-143, hsa-miR-449a, hsa-miR-548m, hsa-miR-183*, hsa-miR-378, hsa-miR-28-5p, hsa-miR-190, hsa-miR-302a*, hsa-miR-191, hsa-miR-129-5p, hsa-miR-541*, hsa-miR-548n, hsa-miR-1178, hsa-miR-217, hsa-miR-302e, hsa-miR-19a*, hsa-miR-1825, hsa-miR-128, hsa-miR-342-3p, hsa-miR-409-5p, hsa-miR-298, hsa-miR-508-5p, hsa-miR-1308, hsa-miR-542-5p, hsa-miR-455-5p, hsa-miR-587, hsa-miR-1207-5p, hsa-miR-623, hsa-miR-875-3p, hsa-miR-657, hsa-miR-216b, hsa-miR-1252, hsa-miR-1182, hsa-miR-630, hsa-miR-580, hsa-miR-148a, hsa-miR-548p, hsa-miR-586, hsa-miR-621, hsa-miR-582-5p, hsa-miR-19b-1*, hsa-miR-143*, hsa-miR-543, hsa-miR-186, hsa-miR-23 a, hsa-miR-449b, hsa-miR-1236, hsa-miR-618, hsa-miR-337-3p, hsa-miR-10a, hsa-miR-33a*, hsa-miR-302c, hsa-miR-606, hsa-miR-520a-5p, hsa-miR-641, hsa-miR-548d-3p, hsa-miR-646, hsa-miR-329, hsa-let-7b*, hsa-miR-151-3p, hsa-miR-708, hsa-miR-590-5p, hsa-miR-145*, hsa-miR-525-5p, hsa-miR-582-3p, hsa-miR-30a*, hsa-miR-371-5p, hsa-miR-29a*, hsa-miR-622, hsa-miR-548o, hsa-miR-1, hsa-miR-376a, hsa-miR-1227, hsa-miR-373, hsa-miR-200c, hsa-miR-581, hsa-miR-616*, hsa-miR-552, hsa-miR-874, hsa-miR-148b, hsa-miR-373*, hsa-miR-548f, hsa-miR-1286, hsa-miR-26b, hsa-miR-720, hsa-miR-664, hsa-miR-484, hsa-miR-573, hsa-miR-32*, hsa-miR-1269, hsa-miR-302d, hsa-miR-1202, hsa-miR-624, hsa-miR-15b, hsa-miR-1324, hsa-miR-320d, hsa-miR-188-3p, hsa-miR-590-3p, hsa-miR-495, hsa-miR-513c, hsa-miR-1297, hsa-miR-591, hsa-miR-7-1*, hsa-miR-589, hsa-miR-215, hsa-miR-767-3p, hsa-miR-589*, hsa-miR-26b*, hsa-miR-1253, hsa-miR-190b, hsa-miR-34a, hsa-miR-135a, hsa-miR-193a-3p, hsa-miR-146a, hsa-miR-768-5p, hsa-miR-1259, hsa-miR-1243, hsa-miR-186*, hsa-miR-147, hsa-miR-873, hsa-miR-520c-3p, hsa-miR-518a-5p, hsa-miR-608, hsa-miR-498, hsa-miR-1226, hsa-miR-1262, hsa-miR-144, hsa-miR-1323, hsa-miR-125b, hsa-miR-323-5p, hsa-miR-653, hsa-miR-220b, hsa-miR-302a, hsa-miR-888, hsa-miR-30d*, hsa-miR-802, hsa-miR-568, hsa-miR-30e*, hsa-miR-592, hsa-miR-21*, hsa-miR-429, hsa-miR-624*, hsa-miR-570, hsa-miR-105, hsa-miR-124, hsa-miR-517*, hsa-miR-940, hsa-miR-34b, hsa-miR-325, hsa-miR-588, hsa-miR-1261, hsa-miR-34b*, hsa-miR-125a-3p, hsa-miR-1285, hsa-miR-512-3p, hsa-miR-520d-3p, hsa-miR-195, hsa-miR-15 a*, hsa-miR-450b-5p, hsa-miR-610, hsa-miR-519d, hsa-miR-198, hsa-miR-144*, hsa-miR-20a*, hsa-miR-545, hsa-miR-181d, hsa-miR-935, hsa-miR-612, hsa-miR-135b, hsa-miR-27b, hsa-miR-664*, hsa-miR-636, hsa-miR-323-3p, hsa-miR-607, hsa-miR-520e, hsa-miR-200a, hsa-miR-578, hsa-miR-10b*, hsa-miR-629*, hsa-miR-93, hsa-miR-383, hsa-miR-29b-2*, hsa-miR-192*, hsa-miR-193b, hsa-miR-566, hsa-miR-518c*, hsa-miR-942, hsa-miR-152, hsa-miR-422a, hsa-miR-23b, hsa-miR-1322, hsa-miR-362-3p, hsa-miR-1263, hsa-miR-539, hsa-miR-548a-3p, hsa-miR-1255a, hsa-miR-146b-5p, hsa-miR-412, hsa-miR-516b, hsa-miR-526b, hsa-miR-33b*, hsa-miR-149*, hsa-miR-302d*, hsa-miR-330-5p, hsa-miR-141, hsa-miR-526b*, hsa-miR-22*, hsa-miR-203, hsa-miR-708*, hsa-miR-597, hsa-miR-744*, hsa-let-7f-1*, hsa-miR-1238, hsa-miR-628-5p, hsa-miR-150, hsa-miR-181c, hsa-miR-501-5p, hsa-miR-205, hsa-miR-224, hsa-miR-1284, hsa-miR-221*, hsa-miR-659, hsa-miR-527, hsa-miR-185, hsa-miR-20b, hsa-miR-378*, hsa-miR-556-5p, hsa-miR-640, hsa-miR-100*, hsa-miR-1224-3p, hsa-miR-199a-3p, hsa-miR-106a, hsa-miR-1245, hsa-miR-138-2*, hsa-miR-877*, hsa-miR-149, hsa-miR-30b*, hsa-miR-130a*, hsa-miR-483-5p, hsa-miR-519e, hsa-miR-520h, hsa-miR-320a, hsa-miR-1225-5p, hsa-miR-10b, hsa-miR-197, hsa-miR-1228, hsa-miR-1827, hsa-miR-24, hsa-miR-1299, hsa-miR-548c-3p, hsa-miR-17, hsa-miR-196a*, hsa-miR-382, hsa-miR-656, hsa-miR-182, hsa-miR-320b, hsa-miR-548k, hsa-miR-675, hsa-miR-424, hsa-miR-506, hsa-miR-302b, hsa-miR-181b, hsa-miR-1283, hsa-miR-548e, hsa-miR-21, hsa-miR-655, hsa-miR-485-5p, hsa-miR-199b-3p, hsa-miR-137, hsa-miR-331-3p, hsa-miR-888*, hsa-miR-488*, hsa-miR-577, hsa-miR-206, hsa-miR-7-2*, hsa-miR-196a, hsa-miR-188-5p, hsa-miR-15a, hsa-miR-485-3p, hsa-miR-632, hsa-miR-1244, hsa-miR-601, hsa-miR-20a, hsa-miR-409-3p, hsa-miR-876-3p, hsa-miR-455-3p, hsa-miR-196b, hsa-let-7a*, hsa-miR-1272, hsa-miR-509-5p, hsa-miR-944, hsa-miR-501-3p, hsa-miR-1265, hsa-miR-193b*, hsa-miR-219-2-3p, hsa-miR-515-3p, hsa-miR-140-3p, hsa-miR-106b, hsa-miR-497, hsa-miR-16-1*, hsa-miR-218, hsa-miR-302b*, hsa-miR-372, hsa-miR-326, hsa-miR-497*, hsa-miR-223*, hsa-miR-433, hsa-miR-505, hsa-miR-595, hsa-miR-154, hsa-miR-26a, hsa-miR-1275, hsa-miR-1255b, hsa-miR-1205, hsa-miR-1302, hsa-miR-891b, hsa-miR-1293, hsa-miR-613, hsa-miR-1225-3p, hsa-miR-154*, hsa-miR-671-5p, hsa-miR-512-5p, hsa-miR-340*, hsa-miR-584.

v-erb-a erythroblastic leukemia viral oncogene homolog 4—ERBB4—NM_{—}001042599—hsa-miR-140-5p, hsa-miR-376b, hsa-miR-557, hsa-miR-665, hsa-miR-637, hsa-miR-200b, hsa-miR-509-3-5p, hsa-miR-490-5p, hsa-miR-642, hsa-miR-448, hsa-miR-500, hsa-miR-515-5p, hsa-miR-141*, hsa-miR-542-3p, hsa-miR-22, hsa-miR-625, hsa-miR-130b*, hsa-miR-216a, hsa-miR-19b-2*, hsa-miR-513a-5p, hsa-miR-650, hsa-miR-1276, hsa-miR-133a, hsa-miR-125b-2*, hsa-miR-520g, hsa-miR-1290, hsa-miR-222*, hsa-miR-374b, hsa-miR-181a, hsa-miR-551b*, hsa-miR-519b-3p, hsa-miR-513b, hsa-miR-182*, hsa-miR-195*, hsa-miR-302c*, hsa-miR-125a-5p, hsa-miR-221, hsa-miR-483-3p, hsa-miR-204, hsa-let-7g*, hsa-miR-377, hsa-miR-30c-1*, hsa-miR-218-1*, hsa-miR-890, hsa-miR-1248, hsa-miR-335, hsa-miR-138-1*, hsa-miR-92a-2*, hsa-miR-452, hsa-miR-16-2*, hsa-miR-496, hsa-miR-320c, hsa-miR-511, hsa-miR-661, hsa-miR-31, hsa-miR-513a-3p, hsa-miR-148a*, hsa-miR-544, hsa-miR-143, hsa-miR-142-5p, hsa-miR-379*, hsa-miR-220a, hsa-miR-24-1*, hsa-miR-335*, hsa-miR-507, hsa-miR-29b-1*, hsa-miR-378, hsa-miR-330-3p, hsa-miR-28-5p, hsa-miR-302a*, hsa-miR-561, hsa-miR-191, hsa-miR-519c-3p, hsa-miR-19b, hsa-miR-129-5p, hsa-miR-943, hsa-miR-541*, hsa-miR-548n, hsa-let-7c*, hsa-miR-603, hsa-miR-19a*, hsa-miR-519a, hsa-miR-342-3p, hsa-miR-1308, hsa-miR-130b, hsa-miR-587, hsa-miR-199a-5p, hsa-miR-1303, hsa-miR-1207-5p, hsa-miR-875-3p, hsa-miR-424*, hsa-miR-216b, hsa-miR-103, hsa-let-7d*, hsa-miR-1252, hsa-miR-630, hsa-miR-660, hsa-miR-1246, hsa-miR-379, hsa-miR-600, hsa-miR-576-5p, hsa-miR-548p, hsa-miR-876-5p, hsa-miR-19b-1*, hsa-miR-524-5p, hsa-miR-30b, hsa-miR-374a*, hsa-miR-186, hsa-miR-23a, hsa-miR-365, hsa-miR-301b, hsa-miR-30c-2*, hsa-miR-1305, hsa-miR-337-3p, hsa-miR-33a*, hsa-miR-491-5p, hsa-miR-302f, hsa-miR-1201, hsa-miR-218-2*, hsa-miR-202, hsa-miR-606, hsa-miR-520a-5p, hsa-miR-410, hsa-miR-421, hsa-miR-24-2*, hsa-miR-548d-3p, hsa-miR-646, hsa-miR-329, hsa-let-7b*, hsa-miR-708, hsa-miR-146a*, hsa-miR-145*, hsa-miR-525-5p, hsa-miR-494, hsa-miR-411, hsa-miR-30a*, hsa-miR-371-5p, hsa-miR-30a, hsa-miR-548o, hsa-miR-376a, hsa-miR-200c, hsa-miR-581, hsa-miR-1207-3p, hsa-miR-130a, hsa-miR-145, hsa-miR-548f, hsa-miR-362-5p, hsa-miR-220c, hsa-miR-26b, hsa-miR-664, hsa-miR-765, hsa-miR-624, hsa-miR-296-5p, hsa-miR-320d, hsa-miR-599, hsa-miR-590-3p, hsa-miR-502-5p, hsa-miR-495, hsa-miR-1297, hsa-miR-7-1*, hsa-miR-939, hsa-miR-589*, hsa-miR-1253, hsa-miR-936, hsa-miR-921, hsa-miR-643, hsa-miR-135a, hsa-miR-579, hsa-miR-411*, hsa-miR-193a-3p, hsa-miR-361-5p, hsa-miR-146a, hsa-miR-768-5p, hsa-miR-1259, hsa-miR-1243, hsa-miR-1233, hsa-miR-518a-5p, hsa-miR-608, hsa-miR-27a*, hsa-miR-498, hsa-miR-1226, hsa-miR-1262, hsa-miR-633, hsa-miR-144, hsa-miR-1323, hsa-miR-125b, hsa-miR-384, hsa-miR-653, hsa-miR-888, hsa-miR-30d*, hsa-miR-568, hsa-miR-30e*, hsa-miR-592, hsa-miR-429, hsa-miR-624*, hsa-miR-570, hsa-miR-105, hsa-miR-508-3p, hsa-miR-517*, hsa-miR-940, hsa-miR-155, hsa-miR-34b, hsa-miR-325, hsa-miR-1261, hsa-miR-222, hsa-miR-432, hsa-miR-1270, hsa-miR-769-3p, hsa-miR-593, hsa-miR-768-3p, hsa-miR-450b-3p, hsa-miR-512-3p, hsa-miR-15a*, hsa-miR-331-5p, hsa-miR-450b-5p, hsa-miR-20a*, hsa-miR-452*, hsa-miR-181d, hsa-miR-135b, hsa-miR-323-3p, hsa-miR-607, hsa-miR-578, hsa-miR-30d, hsa-miR-10b*, hsa-miR-629*, hsa-miR-383, hsa-miR-155*, hsa-miR-193b, hsa-miR-200b*, hsa-miR-518c*, hsa-miR-942, hsa-miR-422a, hsa-miR-23b, hsa-miR-101, hsa-miR-1274a, hsa-miR-362-3p, hsa-miR-545*, hsa-miR-539, hsa-miR-609, hsa-miR-548a-3p, hsa-miR-1255a, hsa-miR-1229, hsa-miR-146b-5p, hsa-miR-634, hsa-miR-133b, hsa-miR-620, hsa-miR-302d*, hsa-miR-330-5p, hsa-miR-551a, hsa-miR-301a, hsa-miR-889, hsa-miR-571, hsa-miR-625*, hsa-miR-708*, hsa-miR-211, hsa-miR-519e*, hsa-let-7f-1*, hsa-miR-432*, hsa-miR-1300, hsa-miR-30c, hsa-miR-181c, hsa-miR-181a-2*, hsa-miR-583, hsa-miR-501-5p, hsa-miR-199b-5p, hsa-miR-205, hsa-miR-527, hsa-miR-378*, hsa-miR-1321, hsa-miR-640, hsa-miR-199a-3p, hsa-miR-18a*, hsa-miR-130a*, hsa-miR-758, hsa-miR-520h, hsa-miR-320a, hsa-miR-339-5p, hsa-miR-1827, hsa-miR-1299, hsa-miR-548c-3p, hsa-miR-135a*, hsa-miR-663b, hsa-miR-1279, hsa-miR-7, hsa-miR-656, hsa-miR-320b, hsa-miR-454, hsa-miR-1264, hsa-miR-297, hsa-miR-548k, hsa-miR-548l, hsa-miR-181b, hsa-miR-1283, hsa-miR-187*, hsa-miR-548e, hsa-miR-1251, hsa-miR-199b-3p, hsa-miR-137, hsa-miR-1278, hsa-miR-331-3p, hsa-miR-888*, hsa-miR-577, hsa-miR-7-2*, hsa-miR-377*, hsa-miR-107, hsa-miR-19a, hsa-miR-188-5p, hsa-miR-485-3p, hsa-miR-374a, hsa-miR-1244, hsa-miR-556-3p, hsa-miR-425, hsa-miR-409-3p, hsa-miR-876-3p, hsa-let-7a*, hsa-miR-509-5p, hsa-miR-944, hsa-miR-340, hsa-miR-1265, hsa-miR-499-5p, hsa-miR-219-2-3p, hsa-miR-140-3p, hsa-let-7f-2*, hsa-miR-1197, hsa-miR-302b*, hsa-miR-551b, hsa-miR-326, hsa-miR-497*, hsa-miR-433, hsa-miR-505, hsa-miR-499-3p, hsa-miR-26a, hsa-miR-1275, hsa-miR-1255b, hsa-miR-142-3p, hsa-miR-520d-5p, hsa-miR-200a*, hsa-miR-558, hsa-miR-1293, hsa-miR-584, hsa-miR-30e.

Epithelial membrane protein 1—EMP1—NM_{—}001423—hsa-miR-557, hsa-miR-877, hsa-miR-1266, hsa-miR-500, hsa-miR-141*, hsa-miR-22, hsa-miR-922, hsa-miR-138, hsa-miR-130b*, hsa-miR-19b-2*, hsa-miR-513a-5p, hsa-miR-1290, hsa-miR-454*, hsa-miR-195*, hsa-miR-337-5p, hsa-miR-204, hsa-let-7g*, hsa-miR-548a-5p, hsa-miR-376c, hsa-miR-647, hsa-miR-559, hsa-miR-1248, hsa-miR-138-1*, hsa-miR-548b-5p, hsa-miR-516a-3p, hsa-miR-16-2*, hsa-miR-496, miR-31, hsa-miR-507, hsa-miR-548m, hsa-miR-330-3p, hsa-miR-190, hsa-miR-129-5p, hsa-miR-548n, hsa-miR-217, hsa-let-7c*, hsa-miR-548c-5p, hsa-miR-19a*, hsa-miR-508-5p, hsa-miR-1303, hsa-miR-1207-5p, hsa-miR-424*, hsa-miR-216b, hsa-miR-148a, hsa-miR-299-5p, hsa-miR-19b-1*, hsa-miR-143*, hsa-miR-34a*, hsa-miR-548i, hsa-miR-606, hsa-miR-548d-3p, hsa-miR-516b*, hsa-miR-324-3p, hsa-miR-548o, hsa-miR-1, hsa-miR-1207-3p, hsa-miR-616*, hsa-miR-148b, hsa-miR-373*, hsa-miR-573, hsa-miR-296-5p, hsa-miR-1324, hsa-miR-188-3p, hsa-miR-96*, hsa-miR-591, hsa-miR-1208, hsa-miR-26b*, hsa-miR-190b, hsa-miR-936, hsa-miR-135a, hsa-miR-1259, hsa-miR-147, hsa-miR-1233, hsa-miR-1226, hsa-miR-185*, hsa-miR-144, hsa-miR-1323, hsa-miR-654-3p, hsa-miR-592, hsa-miR-940, hsa-miR-548d-5p, hsa-miR-1294, hsa-miR-610, hsa-miR-20a*, hsa-miR-135b, hsa-miR-548j, hsa-miR-200a, hsa-miR-10b*, hsa-miR-629*, hsa-miR-518c*, hsa-miR-152, hsa-miR-101, hsa-miR-539, hsa-miR-1255a, hsa-miR-33b, hsa-miR-634, hsa-miR-567, hsa-miR-141, hsa-miR-203, hsa-miR-548h, hsa-miR-708*, hsa-miR-597, hsa-miR-211, hsa-miR-361-3p, hsa-miR-181a-2*, hsa-miR-760, hsa-miR-583, hsa-miR-501-5p, hsa-miR-1321, hsa-miR-486-5p, hsa-miR-548c-3p, hsa-miR-548k, hsa-miR-187*, hsa-miR-206, hsa-miR-33a, hsa-miR-183, hsa-miR-338-3p, hsa-miR-219-2-3p, hsa-miR-367*, hsa-miR-1255b, hsa-miR-1205, hsa-miR-558, hsa-miR-613, hsa-miR-95.

Low Density Lipoprotein receptor-related protein associated protein 1—LRPAP1—NM_{—}002337—hsa-miR-576-3p, hsa-miR-200b, hsa-miR-500, hsa-miR-335, hsa-miR-146b-3p, hsa-miR-1825, hsa-miR-134, hsa-miR-1236, hsa-miR-646, hsa-miR-200c, hsa-miR-1207-3p, hsa-miR-1202, hsa-miR-885-3p, hsa-miR-361-5p, hsa-miR-374b*, hsa-miR-608, hsa-miR-220b, hsa-miR-429, hsa-miR-105, hsa-miR-33b*, hsa-miR-17*, hsa-miR-877*, hsa-miR-519e, hsa-miR-515-3p, hsa-miR-1293, hsa-miR-671-5p.

SCY1-like binding protein 1—SCYL1BP1—NM_{—}152281—hsa-miR-665, hsa-miR-562, hsa-miR-125b-2*, hsa-miR-1290, hsa-miR-101*, hsa-miR-55 1 b*, hsa-miR-513b, hsa-miR-195*, hsa-miR-16-2*, hsa-miR-511, hsa-miR-29b-1*, hsa-miR-191, hsa-miR-194, hsa-miR-548n, hsa-miR-657, hsa-miR-582-5p, hsa-miR-338-5p, hsa-miR-34a*, hsa-miR-186, hsa-miR-618, hsa-miR-33a*, hsa-miR-494, hsa-miR-30a*, hsa-miR-145, hsa-miR-616*, hsa-miR-373*, hsa-miR-26b, hsa-miR-628-3p, hsa-miR-617, hsa-miR-495, hsa-miR-1297, hsa-miR-768-5p, hsa-miR-30d*, hsa-miR-802, hsa-miR-30e*, hsa-miR-34b, hsa-miR-768-3p, hsa-miR-331-5p, hsa-miR-607, hsa-miR-200a, hsa-miR-548b-3p, hsa-miR-545*, hsa-miR-141, hsa-miR-889, hsa-miR-22*, hsa-miR-203, hsa-miR-501-5p, hsa-miR-224, hsa-miR-659, hsa-miR-548c-3p, hsa-miR-382, hsa-miR-656, hsa-miR-548l, hsa-miR-488*, hsa-miR-595, hsa-miR-26a, hsa-miR-891b.

Cleavage stimulation factor, 3′ pre-RNA, subunit 1, 50 kDa—CSTF1—Variant 1, Variant 2 and Variant 3—NM_{—}001033521, NM_{—}001324 and NM_{—}001033522—hsa-miR-510, hsa-miR-509-3-5p, hsa-miR-922, hsa-miR-192, hsa-miR-1184, hsa-miR-1301, hsa-miR-454*, hsa-miR-142-5p, hsa-miR-619, hsa-miR-424*, hsa-miR-548p, hsa-miR-582-5p, hsa-miR-374a*, hsa-miR-365, hsa-miR-337-3p, hsa-miR-410, hsa-miR-421, hsa-let-7b*, hsa-miR-548f, hsa-miR-765, hsa-miR-590-3p, hsa-miR-215, hsa-miR-193a-3p, hsa-miR-361-5p, hsa-miR-653, hsa-miR-545, hsa-miR-935, hsa-miR-607, hsa-miR-193b, hsa-miR-200b*, hsa-miR-548b-3p, hsa-miR-1257, hsa-miR-548a-3p, hsa-miR-567, hsa-let-7f-1*, hsa-miR-583, hsa-miR-486-5p, hsa-miR-196a*, hsa-miR-548e, hsa-miR-1298, hsa-let-7a*, hsa-miR-509-5p, hsa-miR-1275, hsa-miR-200a*, hsa-miR-558, hsa-miR-135b*.

Kelch-like 20—KLHL20—NM_{—}014458—hsa-miR-500, hsa-miR-20b*, hsa-miR-219-1-3p, hsa-miR-19b-2*, hsa-miR-519b-3p, hsa-miR-548g, hsa-miR-30c-1*, hsa-miR-1304, hsa-miR-548a-5p, hsa-miR-559, hsa-miR-548b-5p, hsa-miR-15b*, hsa-miR-544, hsa-miR-29b-1*, hsa-miR-548m, hsa-miR-1237, hsa-miR-519c-3p, hsa-miR-19b, hsa-miR-548c-5p, hsa-miR-19a*, hsa-miR-938, hsa-miR-519a, hsa-miR-130b, hsa-miR-370, hsa-miR-623, hsa-miR-580, hsa-miR-129*, hsa-miR-576-5p, hsa-miR-548p, hsa-miR-19b-1*, hsa-miR-524-5p, hsa-miR-30b, hsa-miR-301b, hsa-miR-30c-2*, hsa-miR-618, hsa-miR-33a*, hsa-miR-548i, hsa-miR-641, hsa-miR-548d-3p, hsa-let-7b*, hsa-miR-30a, hsa-miR-130a, hsa-miR-548f, hsa-miR-362-5p, hsa-miR-664, hsa-miR-96*, hsa-miR-513c, hsa-miR-1253, hsa-miR-768-5p, hsa-miR-18b, hsa-miR-802, hsa-miR-568, hsa-miR-570, hsa-miR-508-3p, hsa-miR-517*, hsa-miR-548d-5p, hsa-miR-432, hsa-miR-125a-3p, hsa-miR-519d, hsa-miR-548j, hsa-miR-578, hsa-miR-30d, hsa-miR-629*, hsa-miR-93, hsa-miR-548b-3p, hsa-miR-942, hsa-miR-548a-3p, hsa-miR-302d*, hsa-miR-301 a, hsa-miR-526b*, hsa-miR-548h, hsa-miR-381, hsa-miR-744*, hsa-let-7f-1*, hsa-miR-432*, hsa-miR-30c, hsa-miR-18a, hsa-miR-20b, hsa-miR-106a, hsa-miR-18a*, hsa-miR-30b*, hsa-miR-129-3p, hsa-miR-17, hsa-miR-656, hsa-miR-454, hsa-miR-1264, hsa-miR-548e, hsa-miR-655, hsa-miR-485-5p, hsa-miR-122, hsa-miR-19a, hsa-miR-188-5p, hsa-miR-300, hsa-miR-20a, hsa-miR-876-3p, hsa-let-7a*, hsa-miR-944, hsa-miR-219-3p, hsa-miR-106b, hsa-miR-302b*, hsa-miR-367*, hsa-miR-1183, hsa-miR-154, hsa-miR-142-3p, hsa-miR-520d-5p, hsa-miR-558, hsa-miR-30e.

DnaJ (Hsp40) homolog, subfamily C, member 13—DNAJC13—NM_{—}015268—hsa-miR-557, hsa-miR-665, hsa-miR-493*, hsa-miR-181a, hsa-miR-204, hsa-miR-96, hsa-miR-27a, hsa-miR-92a-2*, hsa-miR-575, hsa-miR-511, hsa-miR-335*, hsa-miR-507, hsa-miR-330-3p, hsa-miR-561, hsa-miR-129-5p, hsa-miR-943, hsa-miR-651, hsa-miR-30b, hsa-miR-30a, hsa-miR-489, hsa-miR-1, hsa-miR-581, hsa-miR-1207-3p, hsa-miR-569, hsa-miR-590-3p, hsa-miR-1271, hsa-miR-7-1*, hsa-miR-193a-3p, hsa-miR-520f, hsa-miR-633, hsa-miR-568, hsa-miR-588, hsa-miR-34b*, hsa-miR-181d, hsa-miR-27b, hsa-miR-200a, hsa-miR-578, hsa-miR-30d, hsa-miR-193b, hsa-miR-139-5p, hsa-miR-141, hsa-miR-211, hsa-miR-432*, hsa-miR-30c, hsa-miR-181c, hsa-miR-640, hsa-miR-181b, hsa-miR-206, hsa-miR-7-2*, hsa-miR-340, hsa-miR-218, hsa-miR-1183, hsa-miR-613, hsa-miR-30e.

Amyloid beta (A4) precursor-like protein 2—APLP2—NM_{—}001642—hsa-miR-665, hsa-miR-200b, hsa-miR-1254, hsa-miR-16, hsa-miR-448, hsa-miR-1180, hsa-miR-141*, hsa-miR-542-3p, hsa-miR-493*, hsa-miR-219-1-3p, hsa-miR-513a-5p, hsa-miR-513b, hsa-miR-548g, hsa-miR-377, hsa-miR-363*, hsa-miR-335*, hsa-miR-378, hsa-miR-885-5p, hsa-miR-128, hsa-miR-409-5p, hsa-miR-298, hsa-miR-216b, hsa-miR-1252, hsa-miR-1182, hsa-miR-660, hsa-miR-582-5p, hsa-miR-524-5p, hsa-miR-30b, hsa-miR-186, hsa-miR-520a-5p, hsa-miR-212, hsa-miR-153, hsa-miR-525-5p, hsa-miR-582-3p, hsa-miR-30a, hsa-miR-200c, hsa-miR-450a, hsa-miR-765, hsa-miR-532-5p, hsa-miR-15b, hsa-miR-1324, hsa-miR-495, hsa-miR-132, hsa-miR-885-3p, hsa-miR-1243, hsa-miR-608, hsa-miR-592, hsa-miR-596, hsa-miR-429, hsa-miR-503, hsa-miR-570, hsa-miR-517*, hsa-miR-324-5p, hsa-miR-769-3p, hsa-miR-450b-3p, hsa-miR-195, hsa-miR-198, hsa-miR-452*, hsa-miR-200a, hsa-miR-30d, hsa-miR-155*, hsa-miR-942, hsa-miR-422a, hsa-miR-1257, hsa-miR-33b*, hsa-miR-302d*, hsa-miR-139-5p, hsa-miR-141, hsa-miR-30c, hsa-miR-221 *, hsa-miR-1224-3p, hsa-miR-199a-3p, hsa-miR-519e, hsa-miR-548l, hsa-miR-424, hsa-miR-485-5p, hsa-miR-199b-3p, hsa-miR-331-3p, hsa-miR-892a, hsa-miR-15a, hsa-miR-556-3p, hsa-miR-455-3p, hsa-miR-1265, hsa-miR-193b*, hsa-miR-515-3p, hsa-miR-140-3p, hsa-miR-887, hsa-miR-497, hsa-miR-302b*, hsa-miR-520d-5p, hsa-miR-891b, hsa-miR-30e.

Sorbin and SH3 domain containing 2—SORBS2—Variant 1 and Variant 2—NM_{—}003603 and NM_{—}021069—hsa-miR-376b, hsa-miR-557, hsa-miR-518d-5p, hsa-miR-637, hsa-miR-448, hsa-miR-519a*, hsa-miR-500, hsa-miR-522*, hsa-miR-493*, hsa-miR-138, hsa-miR-216a, hsa-miR-1276, hsa-miR-222*, hsa-miR-181a, hsa-miR-551b*, hsa-miR-302c*, hsa-miR-1179, hsa-miR-1248, hsa-miR-127-5p, hsa-miR-452, hsa-miR-320c, hsa-miR-661, hsa-miR-513a-3p, hsa-miR-507, hsa-miR-190, hsa-miR-561, hsa-miR-129-5p, hsa-miR-93*, hsa-miR-603, hsa-miR-128, hsa-miR-619, hsa-miR-587, hsa-miR-623, hsa-miR-1246, hsa-miR-600, hsa-miR-548p, hsa-miR-582-5p, hsa-miR-524-5p, hsa-miR-34a*, hsa-miR-374a*, hsa-miR-186, hsa-miR-23a, hsa-miR-329, hsa-miR-1281, hsa-miR-376a, hsa-miR-548f, hsa-miR-362-5p, hsa-miR-664, hsa-miR-484, hsa-miR-320d, hsa-miR-188-3p, hsa-miR-502-5p, hsa-miR-7-1*, hsa-miR-190b, hsa-miR-936, hsa-miR-643, hsa-miR-579, hsa-miR-518a-5p, hsa-miR-18b, hsa-miR-384, hsa-miR-653, hsa-miR-505*, hsa-miR-596, hsa-miR-503, hsa-miR-34b, hsa-miR-1270, hsa-miR-518f*, hsa-miR-768-3p, hsa-miR-519d, hsa-miR-545, hsa-miR-181d, hsa-miR-935, hsa-miR-636, hsa-miR-520c-5p, hsa-miR-200a, hsa-miR-93, hsa-miR-1206, hsa-miR-200b*, hsa-miR-491-3p, hsa-miR-23b, hsa-miR-1322, hsa-miR-362-3p, hsa-miR-609, hsa-miR-548a-3p, hsa-miR-33b*, hsa-miR-620, hsa-miR-141, hsa-miR-526b*, hsa-miR-203, hsa-miR-518e*, hsa-miR-181c, hsa-miR-526a, hsa-miR-342-5p, hsa-miR-18a, hsa-miR-527, hsa-miR-20b, hsa-miR-523*, hsa-miR-106a, hsa-miR-138-2*, hsa-miR-130a*, hsa-miR-519e, hsa-miR-320a, hsa-miR-17, hsa-miR-196a*, hsa-miR-500*, hsa-miR-1279, hsa-miR-320b, hsa-miR-1264, hsa-miR-297, hsa-miR-548k, hsa-miR-1274b, hsa-miR-181b, hsa-miR-548e, hsa-miR-485-5p, hsa-miR-137, hsa-miR-577, hsa-miR-7-2*, hsa-miR-485-3p, hsa-miR-556-3p, hsa-miR-20a, hsa-miR-876-3p, hsa-miR-126*, hsa-miR-519b-5p, hsa-miR-944, hsa-miR-340, hsa-miR-515-3p, hsa-miR-140-3p, hsa-miR-106b, hsa-miR-519c-5p, hsa-miR-497*, hsa-miR-433, hsa-miR-1275, hsa-miR-520d-5p, hsa-miR-200a*, hsa-miR-891b, hsa-miR-671-5p.

DnaJ (Hsp40) homolog, subfamily B, member 1—DNAJB1—NM_{—}006145—hsa-miR-194*, hsa-miR-665, hsa-miR-509-3-5p, hsa-miR-34c-5p, hsa-miR-500, hsa-miR-380*, hsa-miR-648, hsa-miR-9, hsa-miR-133a, hsa-miR-520g, hsa-miR-181a, hsa-miR-204, hsa-let-7g*, hsa-miR-668, hsa-miR-563, hsa-miR-532-3p, hsa-miR-377, hsa-miR-30c-1*, hsa-miR-647, hsa-miR-920, hsa-miR-544, hsa-miR-486-3p, hsa-miR-449a, hsa-miR-378, hsa-miR-19b, hsa-miR-892b, hsa-miR-603, hsa-miR-128, hsa-miR-370, hsa-miR-623, hsa-miR-875-3p, hsa-miR-657, hsa-miR-1182, hsa-miR-600, hsa-miR-543, hsa-miR-30c-2*, hsa-miR-449b, hsa-miR-202, hsa-miR-641, hsa-miR-548d-3p, hsa-miR-582-3p, hsa-miR-362-5p, hsa-miR-484, hsa-miR-1269, hsa-miR-765, hsa-miR-532-5p, hsa-miR-939, hsa-miR-1253, hsa-miR-34a, hsa-miR-193a-3p, hsa-miR-186*, hsa-miR-220b, hsa-miR-505*, hsa-miR-1224-5p, hsa-miR-570, hsa-miR-508-3p, hsa-miR-155, hsa-miR-34b*, hsa-miR-148b*, hsa-miR-198, hsa-miR-181d, hsa-miR-299-3p, hsa-miR-383, hsa-miR-193b, hsa-miR-518c*, hsa-miR-1260, hsa-miR-422a, hsa-miR-1229, hsa-miR-133b, hsa-miR-149*, hsa-miR-328, hsa-miR-211, hsa-miR-181c, hsa-miR-181a-2*, hsa-miR-1284, hsa-miR-149, hsa-miR-520h, hsa-miR-1299, hsa-miR-181b, hsa-miR-485-5p, hsa-miR-137, hsa-miR-331-3p, hsa-miR-122, hsa-miR-19a, hsa-miR-188-5p, hsa-miR-627, hsa-miR-509-5p, hsa-miR-574-5p, hsa-let-7f-2*, hsa-miR-346, hsa-miR-497*, hsa-miR-662, hsa-miR-1205, hsa-miR-891b.

Nebulette—NEBL—Variant 1 and Variant 2—NM_{—}006393 and NM_{—}213569—hsa-miR-518d-5p, hsa-miR-509-3-5p, hsa-miR-877, hsa-miR-380, hsa-miR-16, hsa-miR-519a*, hsa-miR-500, hsa-miR-542-3p, hsa-miR-522*, hsa-miR-22, hsa-miR-138, hsa-miR-130b*, hsa-miR-216a, hsa-miR-19b-2*, hsa-miR-513a-5p, hsa-miR-766, hsa-miR-9, hsa-miR-133a, hsa-miR-520g, hsa-miR-150*, hsa-miR-1301, hsa-miR-181a, hsa-miR-551b*, hsa-miR-195*, hsa-miR-302c*, hsa-miR-221, hsa-miR-548g, hsa-miR-214, hsa-miR-532-3p, hsa-miR-377, hsa-miR-549, hsa-miR-1826, hsa-miR-647, hsa-miR-27a, hsa-miR-1248, hsa-miR-335, hsa-miR-127-5p, hsa-miR-516a-3p, hsa-miR-452, hsa-miR-16-2*, hsa-miR-496, hsa-miR-320c, hsa-miR-661, hsa-miR-31*, hsa-miR-513a-3p, hsa-miR-99a, hsa-miR-148a*, hsa-miR-335*, hsa-miR-29b-1*, hsa-miR-378, hsa-miR-302a*, hsa-miR-1237, hsa-miR-191, hsa-miR-129-5p, hsa-miR-541*, hsa-miR-548n, hsa-miR-892b, hsa-miR-1288, hsa-let-7c*, hsa-miR-769-5p, hsa-miR-19a*, hsa-miR-508-5p, hsa-miR-619, hsa-miR-1303, hsa-miR-1207-5p, hsa-miR-623, hsa-miR-216b, hsa-miR-148a, hsa-miR-548p, hsa-miR-621, hsa-miR-582-5p, hsa-miR-338-5p, hsa-miR-19b-1*, hsa-miR-524-5p, hsa-miR-30b, hsa-miR-543, hsa-miR-34a*, hsa-miR-374a*, hsa-miR-618, hsa-miR-1305, hsa-miR-337-3p, hsa-miR-33a*, hsa-miR-218-2*, hsa-miR-606, hsa-miR-410, hsa-miR-421, hsa-miR-641, hsa-miR-548d-3p, hsa-miR-516b*, hsa-miR-30a*, hsa-miR-30a, hsa-miR-548o, hsa-miR-581, hsa-miR-616*, hsa-miR-148b, hsa-miR-373*, hsa-miR-548f, hsa-miR-362-5p, hsa-miR-220c, hsa-miR-26b, hsa-miR-569, hsa-miR-664, hsa-miR-484, hsa-miR-573, hsa-miR-1269, hsa-miR-628-3p, hsa-miR-624, hsa-miR-532-5p, hsa-miR-15b, hsa-miR-320d, hsa-miR-96*, hsa-miR-590-3p, hsa-miR-29c, hsa-miR-513c, hsa-miR-1297, hsa-miR-1208, hsa-miR-635, hsa-miR-1200, hsa-miR-29b, hsa-miR-579, hsa-miR-193a-3p, hsa-miR-361-5p, hsa-miR-605, hsa-miR-1243, hsa-miR-374b*, hsa-miR-147, hsa-miR-518a-5p, hsa-miR-18b, hsa-miR-1226, hsa-miR-185*, hsa-miR-1262, hsa-miR-1323, hsa-miR-888, hsa-miR-30d*, hsa-miR-802, hsa-miR-30e*, hsa-miR-596, hsa-miR-29a, hsa-miR-570, hsa-miR-105, hsa-miR-124, hsa-miR-324-5p, hsa-miR-222, hsa-miR-1270, hsa-miR-769-3p, hsa-miR-518f*, hsa-miR-450b-3p, hsa-miR-195, hsa-miR-331-5p, hsa-miR-198, hsa-miR-20a*, hsa-miR-181d, hsa-miR-9*, hsa-miR-27b, hsa-miR-636, hsa-miR-520c-5p, hsa-miR-30d, hsa-miR-383, hsa-miR-155*, hsa-miR-193b, hsa-miR-200b*, hsa-miR-518c*, hsa-miR-548b-3p, hsa-miR-523, hsa-miR-1260, hsa-miR-942, hsa-miR-152, hsa-miR-422a, hsa-miR-1322, hsa-miR-1263, hsa-miR-545*, hsa-miR-539, hsa-miR-1257, hsa-miR-548a-3p, hsa-miR-634, hsa-miR-133b, hsa-miR-567, hsa-miR-149*, hsa-miR-620, hsa-miR-302d*, hsa-miR-223, hsa-miR-889, hsa-miR-203, hsa-miR-625*, hsa-miR-708*, hsa-miR-1185, hsa-miR-744*, hsa-miR-518e*, hsa-miR-1238, hsa-miR-30c, hsa-miR-181c, hsa-miR-526a, hsa-miR-18a, hsa-miR-205, hsa-miR-224, hsa-miR-151-5p, hsa-miR-1284, hsa-miR-527, hsa-miR-378*, hsa-miR-556-5p, hsa-miR-100*, hsa-miR-523*, hsa-miR-631, hsa-miR-1224-3p, hsa-miR-30b*, hsa-miR-99b, hsa-miR-520h, hsa-miR-320a, hsa-miR-486-5p, hsa-miR-202*, hsa-miR-548c-3p, hsa-miR-135a*, hsa-miR-100, hsa-miR-382, hsa-miR-320b, hsa-miR-1264, hsa-miR-548k, hsa-miR-5481, hsa-miR-424, hsa-miR-506, hsa-miR-181b, hsa-miR-1283, hsa-miR-548e, hsa-miR-577, hsa-miR-188-5p, hsa-miR-15a, hsa-miR-485-3p, hsa-miR-423-5p, hsa-miR-126*, hsa-miR-519b-5p, hsa-miR-509-5p, hsa-miR-944, hsa-miR-1265, hsa-miR-193b*, hsa-miR-140-3p, hsa-miR-497, hsa-miR-296-3p, hsa-let-7f-2*, hsa-miR-519c-5p, hsa-miR-218, hsa-miR-302b*, hsa-miR-367*, hsa-miR-595, hsa-miR-26a, hsa-miR-520d-5p, hsa-miR-200a*, hsa-miR-891b, hsa-miR-30e.

SH3 domain binding glutamic acid-rich protein like—SH3BGRL—NM_{—}003022—hsa-miR-448, hsa-miR-130b*, hsa-miR-216a, hsa-miR-221, hsa-miR-890, hsa-miR-548a-5p, hsa-miR-559, hsa-miR-548b-5p, hsa-miR-511, hsa-miR-26a-1*, hsa-miR-548m, hsa-miR-129-5p, hsa-miR-548n, hsa-let-7c*, hsa-miR-548c-5p, hsa-miR-128, hsa-miR-298, hsa-miR-216b, hsa-miR-586, hsa-miR-524-5p, hsa-miR-186, hsa-miR-1305, hsa-miR-33a*, hsa-miR-548i, hsa-miR-606, hsa-miR-548o, hsa-miR-1, hsa-miR-664, hsa-miR-590-3p, hsa-miR-135a, hsa-miR-26a-2*, hsa-miR-520f, hsa-miR-1259, hsa-miR-1323, hsa-miR-488, hsa-miR-548d-5p, hsa-miR-324-5p, hsa-miR-222, hsa-miR-210, hsa-miR-15a*, hsa-miR-135b, hsa-miR-548j, hsa-miR-323-3p, hsa-miR-607, hsa-miR-578, hsa-miR-1206, hsa-miR-609, hsa-miR-634, hsa-miR-889, hsa-miR-203, hsa-miR-548h, hsa-miR-181a-2*, hsa-miR-1284, hsa-miR-138-2*, hsa-miR-106a*, hsa-miR-182, hsa-miR-1264, hsa-miR-548k, hsa-miR-1283, hsa-miR-655, hsa-miR-137, hsa-miR-1278, hsa-miR-206, hsa-miR-196a, hsa-miR-485-3p, hsa-miR-196b, hsa-miR-338-3p, hsa-let-7f-2*, hsa-miR-649, hsa-miR-433, hsa-miR-154, hsa-miR-142-3p, hsa-miR-520d-5p, hsa-miR-891b, hsa-miR-558, hsa-miR-613.

Nudix (nucleoside diphosphate linked moiety X)-type motif 5—NUDT5—NM_{—}014142—hsa-miR-199a-5p, hsa-miR-524-5p, hsa-miR-26b*, hsa-miR-323-5p, hsa-miR-199b-5p, hsa-miR-1264, hsa-miR-218, hsa-miR-520d-5p.

GABA(A) receptor-associated protein like 1—GABARAPL1—NM_{—}031412—hsa-miR-194*, hsa-miR-140-5p, hsa-miR-200b, hsa-miR-877, hsa-miR-16, hsa-miR-130b*, hsa-miR-1276, hsa-miR-133a, hsa-miR-125b-2*, hsa-miR-1301, hsa-miR-551b*, hsa-miR-1179, hsa-miR-532-3p, hsa-miR-30c-1*, hsa-miR-513a-3p, hsa-miR-143, hsa-miR-335*, hsa-miR-548m, hsa-miR-378, hsa-miR-1237, hsa-miR-19b, hsa-miR-1288, hsa-miR-130b, hsa-miR-657, hsa-miR-216b, hsa-miR-103, hsa-miR-548p, hsa-miR-301b, hsa-miR-30c-2*, hsa-miR-33a*, hsa-miR-1201, hsa-miR-646, hsa-miR-329, hsa-let-7b*, hsa-miR-200c, hsa-miR-1207-3p, hsa-miR-130a, hsa-miR-145, hsa-miR-220c, hsa-miR-1269, hsa-miR-15b, hsa-miR-96*, hsa-miR-502-5p, hsa-miR-605, hsa-miR-374b*, hsa-miR-186*, hsa-miR-147, hsa-miR-608, hsa-miR-505*, hsa-miR-654-3p, hsa-miR-429, hsa-miR-155, hsa-miR-768-3p, hsa-miR-195, hsa-miR-545, hsa-miR-629*, hsa-miR-934, hsa-miR-548b-3p, hsa-miR-1260, hsa-miR-422a, hsa-miR-1274a, hsa-miR-362-3p, hsa-miR-412, hsa-miR-133b, hsa-miR-301a, hsa-miR-203, hsa-miR-381, hsa-miR-593*, hsa-let-7f-1*, hsa-miR-205, hsa-miR-17*, hsa-miR-378*, hsa-miR-1256, hsa-miR-339-5p, hsa-miR-454, hsa-miR-424, hsa-miR-331-3p, hsa-miR-888*, hsa-miR-107, hsa-miR-19a, hsa-miR-300, hsa-miR-15a, hsa-let-7a*, hsa-miR-944, hsa-miR-497, hsa-miR-595, hsa-miR-1293.

Microtubule-associated protein tau—MAPT—Variant 1, Variant 2, Variant 3 and Variant 4—NM_{—}016835, NM_{—}005910, NM_{—}016834 and NM_{—}016841—hsa-miR-132*, hsa-miR-453, hsa-miR-518d-5p, hsa-miR-637, hsa-miR-510, hsa-miR-509-3-5p, hsa-miR-520b, hsa-miR-1266, hsa-miR-34c-5p, hsa-miR-1254, hsa-miR-642, hsa-miR-519a*, hsa-miR-500, hsa-miR-380*, hsa-miR-522*, hsa-miR-22, hsa-miR-922, hsa-miR-625, hsa-miR-19b-2*, hsa-miR-766, hsa-miR-650, hsa-miR-125b-2*, hsa-miR-520g, hsa-miR-615-5p, hsa-miR-181a, hsa-miR-513b, hsa-miR-337-5p, hsa-miR-548g, hsa-miR-204, hsa-miR-96, hsa-miR-604, hsa-miR-668, hsa-miR-563, hsa-miR-214, hsa-miR-532-3p, hsa-miR-377, hsa-miR-30c-1*, hsa-miR-923, hsa-miR-890, hsa-miR-647, hsa-miR-335, hsa-miR-920, hsa-miR-363*, hsa-miR-516a-3p, hsa-miR-575, hsa-miR-320c, hsa-miR-520a-3p, hsa-miR-661, hsa-miR-142-5p, hsa-miR-486-3p, hsa-miR-449a, hsa-miR-378, hsa-miR-330-3p, hsa-miR-892b, hsa-miR-1288, hsa-miR-302e, hsa-miR-93*, hsa-miR-603, hsa-miR-19a*, hsa-miR-1825, hsa-miR-134, hsa-miR-342-3p, hsa-miR-508-5p, hsa-miR-1231, hsa-miR-663, hsa-miR-370, hsa-miR-199a-5p, hsa-miR-1207-5p, hsa-miR-623, hsa-miR-657, hsa-miR-876-5p, hsa-miR-615-3p, hsa-miR-19b-1*, hsa-miR-524-5p, hsa-miR-186, hsa-miR-30c-2*, hsa-miR-449b, hsa-miR-33a*, hsa-miR-302c, hsa-miR-302f, hsa-miR-520a-5p, hsa-miR-641, hsa-miR-212, hsa-miR-516b*, hsa-miR-646, hsa-miR-329, hsa-miR-525-5p, hsa-miR-582-3p, hsa-miR-489, hsa-miR-29a*, hsa-miR-622, hsa-miR-324-3p, hsa-miR-548o, hsa-miR-1227, hsa-miR-373, hsa-miR-1207-3p, hsa-miR-874, hsa-miR-220c, hsa-miR-302d, hsa-miR-1202, hsa-miR-765, hsa-miR-624, hsa-miR-1324, hsa-miR-320d, hsa-miR-188-3p, hsa-miR-617, hsa-miR-1271, hsa-miR-513c, hsa-miR-132, hsa-miR-589, hsa-miR-635, hsa-miR-767-3p, hsa-miR-589*, hsa-miR-1291, hsa-miR-34a, hsa-miR-135a, hsa-miR-579, hsa-miR-146a, hsa-miR-605, hsa-miR-520c-3p, hsa-miR-518a-5p, hsa-miR-608, hsa-miR-27a*, hsa-miR-144, hsa-miR-1323, hsa-miR-214*, hsa-miR-220b, hsa-miR-505*, hsa-miR-302a, hsa-miR-596, hsa-miR-503, hsa-miR-517*, hsa-miR-940, hsa-miR-432, hsa-miR-593, hsa-miR-136*, hsa-miR-518f*, hsa-miR-34b*, hsa-miR-148b*, hsa-miR-125a-3p, hsa-miR-512-3p, hsa-miR-520d-3p, hsa-miR-18b*, hsa-miR-450b-5p, hsa-miR-181d, hsa-miR-135b, hsa-miR-1204, hsa-miR-9*, hsa-miR-607, hsa-miR-520e, hsa-miR-520c-5p, hsa-miR-578, hsa-miR-629*, hsa-miR-934, hsa-miR-200b*, hsa-miR-548b-3p, hsa-miR-942, hsa-miR-422a, hsa-miR-362-3p, hsa-miR-539, hsa-miR-1255a, hsa-miR-1229, hsa-miR-146b-5p, hsa-miR-516b, hsa-miR-149*, hsa-miR-302d*, hsa-miR-492, hsa-miR-211, hsa-miR-361-3p, hsa-miR-518e*, hsa-miR-593*, hsa-miR-1300, hsa-miR-150, hsa-miR-181c, hsa-miR-526a, hsa-miR-181a-2*, hsa-miR-760, hsa-miR-583, hsa-miR-501-5p, hsa-miR-199b-5p, hsa-miR-151-5p, hsa-miR-527, hsa-miR-17*, hsa-miR-378*, hsa-miR-1321, hsa-miR-523*, hsa-miR-1245, hsa-miR-877*, hsa-miR-149, hsa-miR-18a*, hsa-miR-30b*, hsa-miR-191*, hsa-miR-550, hsa-miR-758, hsa-miR-520h, hsa-miR-320a, hsa-miR-197, hsa-miR-1827, hsa-miR-24, hsa-miR-196a*, hsa-miR-663b, hsa-miR-500*, hsa-miR-320b, hsa-miR-302b, hsa-miR-626, hsa-miR-181b, hsa-miR-187*, hsa-miR-1251, hsa-miR-485-5p, hsa-miR-888*, hsa-miR-377*, hsa-miR-188-5p, hsa-miR-219-5p, hsa-miR-409-3p, hsa-miR-876-3p, hsa-miR-519b-5p, hsa-miR-509-5p, hsa-miR-944, hsa-miR-887, hsa-miR-296-3p, hsa-miR-1197, hsa-miR-519c-5p, hsa-miR-346, hsa-miR-218, hsa-miR-302b*, hsa-miR-372, hsa-miR-505, hsa-miR-1275, hsa-miR-1255b, hsa-miR-520d-5p, hsa-miR-200a*, hsa-miR-1205, hsa-miR-1302, hsa-miR-558, hsa-miR-1293, hsa-miR-1225-3p, hsa-miR-671-5p, hsa-miR-512-5p.

Discoidin, CUB and LCCL domain containing 1—DCBLD1—NM_{—}173674—hsa-miR-658, hsa-miR-93*, hsa-miR-370, hsa-miR-524-5p, hsa-miR-517*, hsa-miR-18a*, hsa-miR-520d-5p, hsa-miR-95.

Serine threonine kinase 39—STK39—NM_{—}013233—hsa-miR-517a, hsa-miR-877, hsa-miR-32, hsa-miR-642, hsa-miR-22, hsa-miR-1276, hsa-miR-616, hsa-miR-1301, hsa-miR-302c*, hsa-miR-1179, hsa-miR-27a, hsa-miR-92a-2*, hsa-miR-513a-3p, hsa-miR-544, hsa-miR-561, hsa-miR-92b, hsa-miR-587, hsa-miR-630, hsa-miR-299-5p, hsa-miR-548p, hsa-miR-367, hsa-miR-30b, hsa-miR-25, hsa-miR-186, hsa-miR-520a-5p, hsa-miR-212, hsa-miR-525-5p, hsa-miR-30a, hsa-miR-489, hsa-miR-29a*, hsa-miR-548o, hsa-miR-26b, hsa-miR-32*, hsa-miR-590-3p, hsa-miR-132, hsa-miR-1297, hsa-miR-7-1*, hsa-miR-1259, hsa-miR-608, hsa-miR-1323, hsa-miR-653, hsa-miR-553, hsa-miR-488, hsa-miR-508-3p, hsa-miR-124, hsa-miR-517*, hsa-miR-34b*, hsa-miR-450b-5p, hsa-miR-27b, hsa-miR-578, hsa-miR-30d, hsa-miR-10b*, hsa-miR-192*, hsa-miR-1263, hsa-miR-539, hsa-miR-149*, hsa-miR-223, hsa-miR-92a, hsa-miR-708*, hsa-miR-744*, hsa-miR-150, hsa-miR-30c, hsa-miR-583, hsa-miR-1321, hsa-miR-1224-3p, hsa-miR-877*, hsa-miR-30b*, hsa-miR-1827, hsa-miR-196a*, hsa-miR-5481, hsa-miR-506, hsa-miR-122, hsa-miR-7-2*, hsa-miR-363, hsa-miR-944, hsa-miR-296-3p, hsa-let-7f-2*, hsa-miR-517c, hsa-miR-16-1*, hsa-miR-223*, hsa-miR-26a, hsa-miR-558, hsa-miR-30e.

Protein kinase N2—PKN2—NM_{—}006256—hsa-miR-576-3p, hsa-miR-124*, hsa-miR-376b, hsa-miR-518d-5p, hsa-miR-510, hsa-miR-200b, hsa-miR-509-3-5p, hsa-miR-520b, hsa-miR-16, hsa-miR-519a*, hsa-miR-1306, hsa-miR-515-5p, hsa-miR-542-3p, hsa-miR-522*, hsa-miR-922, hsa-miR-625, hsa-miR-130b*, hsa-miR-648, hsa-miR-216a, hsa-miR-766, hsa-miR-1276, hsa-miR-1184, hsa-miR-9, hsa-miR-520g, hsa-miR-1290, hsa-miR-374b, hsa-miR-1301, hsa-miR-551b*, hsa-miR-513b, hsa-miR-221, hsa-miR-96, hsa-miR-28-3p, hsa-miR-377, hsa-miR-218-1*, hsa-miR-548a-5p, hsa-miR-559, hsa-miR-27a, hsa-miR-548b-5p, hsa-miR-452, hsa-miR-496, hsa-miR-520a-3p, hsa-miR-511, hsa-miR-661, hsa-miR-513 a-3p, hsa-miR-148a*, hsa-miR-379*, hsa-miR-335*, hsa-miR-548m, hsa-miR-28-5p, hsa-miR-1237, hsa-miR-561, hsa-miR-129-5p, hsa-miR-658, hsa-miR-302e, hsa-miR-93*, hsa-miR-548c-5p, hsa-miR-886-3p, hsa-miR-603, hsa-miR-342-3p, hsa-miR-298, hsa-miR-508-5p, hsa-miR-455-5p, hsa-miR-216b, hsa-miR-580, hsa-miR-129*, hsa-miR-576-5p, hsa-miR-548p, hsa-miR-582-5p, hsa-miR-524-5p, hsa-miR-143*, hsa-miR-543, hsa-miR-186, hsa-miR-23a, hsa-miR-1305, hsa-miR-33a*, hsa-miR-302c, hsa-miR-302f, hsa-miR-548i, hsa-miR-606, hsa-miR-520a-5p, hsa-miR-641, hsa-miR-212, hsa-miR-548d-3p, hsa-let-7b*, hsa-miR-151-3p, hsa-miR-708, hsa-miR-146a*, hsa-miR-525-5p, hsa-miR-29a*, hsa-miR-548o, hsa-miR-376a, hsa-miR-373, hsa-miR-200c, hsa-miR-1207-3p, hsa-miR-616*, hsa-miR-373*, hsa-miR-548f, hsa-miR-26b, hsa-miR-569, hsa-miR-302d, hsa-miR-1202, hsa-miR-15b, hsa-miR-96*, hsa-miR-590-3p, hsa-miR-495, hsa-miR-1271, hsa-miR-132, hsa-miR-1297, hsa-miR-7-1*, hsa-miR-34c-3p, hsa-miR-26b*, hsa-miR-1291, hsa-miR-1253, hsa-miR-579, hsa-miR-411*, hsa-miR-361-5p, hsa-miR-605, hsa-miR-186*, hsa-miR-1233, hsa-miR-520c-3p, hsa-miR-144, hsa-miR-1323, hsa-miR-384, hsa-miR-653, hsa-miR-302a, hsa-miR-888, hsa-miR-568, hsa-miR-429, hsa-miR-503, hsa-miR-570, hsa-miR-105, hsa-miR-155, hsa-miR-548d-5p, hsa-miR-34b, hsa-miR-1294, hsa-miR-222, hsa-miR-769-3p, hsa-miR-518f*, hsa-miR-34b*, hsa-miR-148b*, hsa-miR-1285, hsa-miR-450b-3p, hsa-miR-520d-3p, hsa-miR-18b*, hsa-miR-195, hsa-miR-450b-5p, hsa-miR-545, hsa-miR-612, hsa-miR-514, hsa-miR-27b, hsa-miR-664*, hsa-miR-636, hsa-miR-548j, hsa-miR-520e, hsa-miR-520c-5p, hsa-miR-578, hsa-miR-383, hsa-miR-554, hsa-miR-491-3p, hsa-miR-942, hsa-miR-23b, hsa-miR-1257, hsa-miR-548a-3p, hsa-miR-33b*, hsa-miR-149*, hsa-miR-328, hsa-miR-139-5p, hsa-miR-223, hsa-miR-571, hsa-miR-548h, hsa-miR-519e*, hsa-miR-518e*, hsa-let-7f-1*, hsa-miR-628-5p, hsa-miR-526a, hsa-miR-224, hsa-miR-1284, hsa-miR-659, hsa-miR-185, hsa-miR-1321, hsa-miR-523*, hsa-miR-631, hsa-miR-138-2*, hsa-miR-30b*, hsa-miR-130a*, hsa-miR-519e, hsa-miR-550, hsa-miR-520h, hsa-miR-129-3p, hsa-miR-548c-3p, hsa-miR-7, hsa-miR-656, hsa-miR-5481, hsa-miR-424, hsa-miR-654-5p, hsa-miR-302b, hsa-miR-1283, hsa-miR-548e, hsa-miR-485-5p, hsa-miR-137, hsa-miR-888*, hsa-miR-7-2*, hsa-miR-196a, hsa-miR-377*, hsa-miR-15a, hsa-miR-374a, hsa-miR-425, hsa-miR-876-3p, hsa-miR-455-3p, hsa-miR-196b, hsa-miR-126*, hsa-miR-519b-5p, hsa-let-7a*, hsa-miR-509-5p, hsa-miR-944, hsa-miR-515-3p, hsa-miR-140-3p, hsa-miR-887, hsa-miR-497, hsa-miR-369-3p, hsa-let-7f-2*, hsa-miR-519c-5p, hsa-miR-218, hsa-miR-372, hsa-miR-497*, hsa-miR-433, hsa-miR-505, hsa-miR-595, hsa-miR-154, hsa-miR-26a, hsa-miR-1275, hsa-miR-541, hsa-miR-520d-5p, hsa-miR-1205.

Casein kinase 2, alpha 1 polypeptide—CSNK2A1—Variant 1, Variant 2 and Variant 3—NM_{—}177559, NM_{—}001895 and NM_{—}177560—hsa-miR-642, hsa-miR-518e, hsa-miR-644, hsa-miR-125a-5p, hsa-miR-221, hsa-let-7g*, hsa-miR-30c-1*, hsa-miR-1304, hsa-miR-1248, hsa-miR-335, hsa-miR-363*, hsa-miR-661, hsa-miR-143, hsa-miR-379*, hsa-miR-29b-1*, hsa-miR-548n, hsa-miR-603, hsa-miR-663, hsa-miR-216b, hsa-miR-1296, hsa-miR-34a*, hsa-miR-186, hsa-miR-30c-2*, hsa-miR-1305, hsa-miR-337-3p, hsa-miR-33a*, hsa-miR-606, hsa-miR-641, hsa-miR-92b*, hsa-miR-573, hsa-miR-188-3p, hsa-miR-513c, hsa-miR-767-3p, hsa-miR-1253, hsa-miR-411*, hsa-miR-1243, hsa-miR-608, hsa-miR-185*, hsa-miR-125b, hsa-miR-588, hsa-miR-222, hsa-miR-1270, hsa-miR-20a*, hsa-miR-200a, hsa-miR-518c*, hsa-miR-634, hsa-miR-516b, hsa-miR-149*, hsa-miR-620, hsa-miR-141, hsa-miR-571, hsa-miR-432*, hsa-miR-1300, hsa-miR-181a-2*, hsa-miR-760, hsa-miR-1224-3p, hsa-miR-30b*, hsa-miR-486-5p, hsa-miR-5481, hsa-miR-1251, hsa-miR-485-5p, hsa-miR-632, hsa-miR-455-3p, hsa-miR-649, hsa-miR-595, hsa-miR-1250, hsa-miR-1205, hsa-miR-744.

Paired immunoglobin-like type 2 receptor beta—PILRB—Variant 1 and Variant 3—NM_{—}013440 and NM_{—}178238—hsa-miR-140-5p, hsa-miR-557, hsa-miR-1266, hsa-miR-16, hsa-miR-374b, hsa-miR-513b, hsa-miR-507, hsa-miR-1237, hsa-miR-103, hsa-miR-548p, hsa-miR-337-3p, hsa-miR-362-5p, hsa-miR-15b, hsa-miR-105, hsa-miR-195, hsa-miR-629*, hsa-miR-192*, hsa-miR-1292, hsa-miR-1185, hsa-miR-151-5p, hsa-miR-424, hsa-miR-655, hsa-miR-107, hsa-miR-188-5p, hsa-miR-15a, hsa-miR-374a, hsa-miR-574-5p, hsa-miR-497, hsa-miR-499-3p.

PILRB—Variant 2—NM_{—}175047—hsa-miR-140-5p, hsa-miR-557, hsa-miR-1266, hsa-miR-16, hsa-miR-374b, hsa-miR-513b, hsa-miR-507, hsa-miR-1237, hsa-miR-103, hsa-miR-548p, hsa-miR-337-3p, hsa-miR-362-5p, hsa-miR-15b, hsa-miR-105, hsa-miR-195, hsa-miR-629*, hsa-miR-192*, hsa-miR-1292, hsa-miR-1185, hsa-miR-224, hsa-miR-151-5p, hsa-miR-424, hsa-miR-655, hsa-miR-107, hsa-miR-188-5p, hsa-miR-15a, hsa-miR-374a, hsa-miR-574-5p, hsa-miR-497, hsa-miR-499-3p.

Endoplasmic reticulum to nucleus signaling 1—ERN1—NM_{—}001433—hsa-miR-665, hsa-miR-637, hsa-miR-509-3-5p, hsa-miR-642, hsa-miR-141*, hsa-miR-216a, hsa-miR-650, hsa-miR-193a-5p, hsa-miR-532-3p, hsa-miR-24-1*, hsa-miR-769-5p, hsa-miR-342-3p, hsa-miR-1231, hsa-miR-370, hsa-miR-548p, hsa-miR-24-2*, hsa-miR-329, hsa-miR-151-3p, hsa-miR-146a*, hsa-miR-153, hsa-miR-1207-3p, hsa-miR-1286, hsa-miR-720, hsa-miR-1202, hsa-miR-29c, hsa-miR-1208, hsa-miR-589*, hsa-miR-26b*, hsa-miR-1200, hsa-miR-936, hsa-miR-29b, hsa-miR-518a-5p, hsa-miR-220b, hsa-miR-29a, hsa-miR-488, hsa-miR-124, hsa-miR-324-5p, hsa-miR-588, hsa-miR-593, hsa-miR-1285, hsa-miR-612, hsa-miR-629*, hsa-miR-1274a, hsa-miR-362-3p, hsa-miR-539, hsa-miR-330-5p, hsa-miR-342-5p, hsa-miR-527, hsa-miR-631, hsa-miR-1224-3p, hsa-miR-877*, hsa-miR-339-5p, hsa-miR-1228, hsa-miR-506, hsa-miR-626, hsa-miR-888*, hsa-miR-423-5p, hsa-miR-338-3p, hsa-miR-509-5p, hsa-miR-639, hsa-miR-326, hsa-miR-1205, hsa-miR-671-5p.

Serum/gluco-corticoid regulated kinase family, member 3—SGK3—Variant 1, Variant 2 and Variant 3—NM_{—}013257, NM_{—}170709 and NM_{—}001033578—hsa-miR-520b, hsa-miR-32, hsa-miR-645, hsa-miR-20b*, hsa-miR-515-5p, hsa-miR-562, hsa-miR-1276, hsa-miR-520g, hsa-miR-550*, hsa-miR-181a, hsa-miR-519b-3p, hsa-miR-195*, hsa-miR-487a, hsa-miR-548g, hsa-miR-377, hsa-miR-376c, hsa-miR-16-2*, hsa-miR-496, hsa-miR-520a-3p, hsa-miR-148a*, hsa-miR-142-5p, hsa-miR-335*, hsa-miR-548m, hsa-miR-302a*, hsa-miR-519c-3p, hsa-miR-129-5p, hsa-miR-943, hsa-miR-302e, hsa-miR-93*, hsa-let-7c*, hsa-miR-519a, hsa-miR-92b, hsa-miR-130b, hsa-miR-1231, hsa-miR-455-5p, hsa-miR-1182, hsa-miR-600, hsa-miR-548p, hsa-miR-367, hsa-miR-338-5p, hsa-miR-524-5p, hsa-miR-143*, hsa-miR-543, hsa-miR-25, hsa-miR-365, hsa-miR-301b, hsa-miR-1305, hsa-miR-33a*, hsa-miR-302c, hsa-miR-1201, hsa-miR-520a-5p, hsa-miR-641, hsa-miR-212, hsa-miR-548d-3p, hsa-let-7b*, hsa-miR-153, hsa-miR-590-5p, hsa-miR-525-5p, hsa-miR-1281, hsa-miR-494, hsa-miR-582-3p, hsa-miR-30a*, hsa-miR-371-5p, hsa-miR-29a*, hsa-miR-548o, hsa-miR-1227, hsa-miR-373, hsa-miR-130a, hsa-miR-548f, hsa-miR-569, hsa-miR-720, hsa-miR-664, hsa-miR-302d, hsa-miR-590-3p, hsa-miR-513c, hsa-miR-132, hsa-miR-7-1*, hsa-miR-1200, hsa-miR-1259, hsa-miR-186*, hsa-miR-200c*, hsa-miR-520c-3p, hsa-miR-770-5p, hsa-miR-498, hsa-miR-144, hsa-miR-1323, hsa-miR-302a, hsa-miR-30d*, hsa-miR-654-3p, hsa-miR-30e*, hsa-miR-940, hsa-miR-155, hsa-miR-432, hsa-miR-1270, hsa-miR-125a-3p, hsa-miR-512-3p, hsa-miR-520d-3p, hsa-miR-450b-5p, hsa-miR-519d, hsa-miR-181d, hsa-miR-664*, hsa-miR-520e, hsa-miR-1258, hsa-miR-578, hsa-miR-93, hsa-miR-29b-2*, hsa-miR-192*, hsa-miR-1263, hsa-miR-539, hsa-miR-548a-3p, hsa-miR-620, hsa-miR-301a, hsa-miR-526b*, hsa-miR-22*, hsa-miR-203, hsa-miR-92a, hsa-miR-381, hsa-miR-708*, hsa-miR-597, hsa-miR-744*, hsa-miR-519e*, hsa-let-7f-1*, hsa-miR-150, hsa-miR-181c, hsa-miR-224, hsa-miR-20b, hsa-miR-378*, hsa-miR-199a-3p, hsa-miR-106a, hsa-miR-1245, hsa-miR-483-5p, hsa-miR-520h, hsa-miR-548c-3p, hsa-miR-17, hsa-miR-106a*, hsa-miR-656, hsa-miR-454, hsa-miR-1264, hsa-miR-302b, hsa-miR-181b, hsa-miR-548e, hsa-miR-21, hsa-miR-199b-3p, hsa-miR-7-2*, hsa-miR-300, hsa-miR-485-3p, hsa-miR-556-3p, hsa-miR-20a, hsa-miR-126*, hsa-miR-363, hsa-let-7a*, hsa-miR-193b*, hsa-miR-106b, hsa-miR-218, hsa-miR-372, hsa-miR-223*, hsa-miR-1234, hsa-miR-520d-5p, hsa-miR-89 1 b, hsa-miR-154*.

WEE1 homolog—WEE1—NM_{—}003390—hsa-miR-194*, hsa-miR-140-5p, hsa-miR-124*, hsa-miR-520b, hsa-miR-16, hsa-miR-138, hsa-miR-875-5p, hsa-miR-125b-2*, hsa-miR-520g, hsa-miR-519b-3p, hsa-miR-513b, hsa-miR-204, hsa-let-7g*, hsa-miR-1179, hsa-miR-890, hsa-miR-548a-5p, hsa-miR-559, hsa-miR-27a, hsa-miR-548b-5p, hsa-miR-516a-3p, hsa-miR-575, hsa-miR-520a-3p, hsa-miR-15b*, hsa-miR-335*, hsa-miR-519c-3p, hsa-miR-19b, hsa-miR-302e, hsa-miR-548c-5p, hsa-miR-128, hsa-miR-519a, hsa-miR-130b, hsa-miR-875-3p, hsa-miR-129*, hsa-miR-524-5p, hsa-miR-301b, hsa-miR-1305, hsa-miR-302c, hsa-miR-548i, hsa-miR-218-2*, hsa-miR-606, hsa-miR-548d-3p, hsa-miR-516b*, hsa-miR-646, hsa-let-7b*, hsa-miR-548o, hsa-miR-1, hsa-miR-373, hsa-miR-130a, hsa-miR-26b, hsa-miR-302d, hsa-miR-15b, hsa-miR-495, hsa-miR-1297, hsa-miR-589*, hsa-miR-1243, hsa-miR-520c-3p, hsa-miR-144, hsa-miR-1323, hsa-miR-220b, hsa-miR-302a, hsa-miR-654-3p, hsa-miR-570, hsa-miR-155, hsa-miR-548d-5p, hsa-miR-520d-3p, hsa-miR-195, hsa-miR-519d, hsa-miR-545, hsa-miR-27b, hsa-miR-548j, hsa-miR-323-3p, hsa-miR-520e, hsa-miR-93, hsa-miR-155*, hsa-miR-192*, hsa-miR-1206, hsa-miR-545*, hsa-miR-149*, hsa-miR-301a, hsa-miR-526b*, hsa-miR-548h, hsa-miR-381, hsa-miR-211, hsa-let-7f-1*, hsa-miR-1284, hsa-miR-20b, hsa-miR-1321, hsa-miR-106a, hsa-miR-520h, hsa-miR-129-3p, hsa-miR-17, hsa-miR-106a*, hsa-miR-454, hsa-miR-424, hsa-miR-302b, hsa-miR-1283, hsa-miR-187*, hsa-miR-655, hsa-miR-888*, hsa-miR-924, hsa-miR-577, hsa-miR-206, hsa-miR-19a, hsa-miR-300, hsa-miR-15a, hsa-miR-20a, hsa-miR-627, hsa-miR-126*, hsa-let-7a*, hsa-miR-944, hsa-miR-140-3p, hsa-miR-106b, hsa-miR-497, hsa-miR-369-3p, hsa-miR-16-1*, hsa-miR-372, hsa-miR-26a, hsa-miR-520d-5p, hsa-miR-613.

Microtubule associated serine/threonine kinase family member 4—MAST4—Variant 1—NM_{—}015183—hsa-miR-511, hsa-miR-29b-1*, hsa-miR-548m, hsa-miR-194, hsa-miR-128, hsa-miR-324-3p, hsa-miR-188-3p, hsa-miR-488, hsa-miR-512-3p, hsa-miR-493, hsa-miR-339-5p, hsa-miR-1299, hsa-miR-331-3p, hsa-miR-196a, hsa-miR-196b.

MAST4—Variant 2—NM_{—}198828—hsa-miR-190, hsa-miR-190b.

Chromosome 11 open reading frame 17—- C11orf17—Variant 1—Variant 2—NM_{—}182901—NM_{—}020642—hsa-miR-34c-5p, hsa-miR-20b*, hsa-miR-766, hsa-miR-487a, hsa-miR-890, hsa-miR-363*, hsa-miR-661, hsa-miR-15b*, hsa-miR-449a, hsa-miR-886-3p, hsa-miR-508-5p, hsa-miR-216b, hsa-miR-449b, hsa-miR-548d-3p, hsa-miR-145, hsa-miR-616*, hsa-miR-373*, hsa-miR-1202, hsa-miR-1324, hsa-miR-495, hsa-miR-34a, hsa-miR-124, hsa-miR-940, hsa-miR-1285, hsa-miR-612, hsa-miR-545*, hsa-miR-30b*, hsa-miR-550, hsa-miR-1827, hsa-miR-196a*, hsa-miR-506, hsa-miR-485-5p, hsa-miR-485-3p, hsa-miR-649, hsa-miR-505, hsa-miR-154*, hsa-miR-340*.

Nucleoproin 37 kDa—NUP37—NM_{—}024057—hsa-miR-561, hsa-miR-524-5p, hsa-miR-570, hsa-miR-625*, hsa-let-7e*, hsa-miR-5481, hsa-miR-520d-5p.

Clathrin, heavy chain (Hc)—CLTC—NM_{—}004859—hsa-miR-140-5p, hsa-miR-557, hsa-miR-520b, hsa-miR-1266, hsa-miR-27b*, hsa-miR-625, hsa-miR-219-1-3p, hsa-miR-513a-5p, hsa-miR-1276, hsa-miR-520g, hsa-miR-616, hsa-miR-1290, hsa-miR-551b*, hsa-miR-519b-3p, hsa-miR-182*, hsa-miR-195*, hsa-miR-302c*, hsa-miR-204, hsa-miR-96, hsa-miR-532-3p, hsa-miR-377, hsa-miR-647, hsa-miR-1248, hsa-miR-335, hsa-miR-92a-2*, hsa-miR-516a-3p, hsa-miR-16-2*, hsa-miR-496, hsa-miR-520a-3p, hsa-miR-511, hsa-miR-15b*, hsa-miR-513a-3p, hsa-miR-335*, hsa-miR-507, hsa-miR-548m, hsa-miR-330-3p, hsa-miR-561, hsa-miR-519c-3p, hsa-miR-19b, hsa-miR-129-5p, hsa-miR-892b, hsa-miR-302e, hsa-miR-574-3p, hsa-miR-519a, hsa-miR-130b, hsa-miR-199a-5p, hsa-miR-1287, hsa-miR-1182, hsa-miR-630, hsa-miR-148a, hsa-miR-576-5p, hsa-miR-548p, hsa-miR-586, hsa-miR-338-5p, hsa-miR-30b, hsa-miR-143*, hsa-miR-543, hsa-miR-34a*, hsa-miR-23a, hsa-miR-301b, hsa-miR-1236, hsa-miR-33a*, hsa-miR-302c, hsa-miR-491-5p, hsa-miR-606, hsa-miR-410, hsa-miR-421, hsa-miR-548d-3p, hsa-miR-516b*, hsa-miR-646, hsa-miR-151-3p, hsa-miR-153, hsa-miR-590-5p, hsa-miR-30a*, hsa-miR-30a, hsa-miR-29a*, hsa-miR-548o, hsa-miR-1, hsa-miR-373, hsa-miR-130a, hsa-miR-148b, hsa-miR-548f, hsa-miR-220c, hsa-miR-569, hsa-miR-664, hsa-miR-573, hsa-miR-302d, hsa-miR-188-3p, hsa-miR-1271, hsa-miR-29c, hsa-miR-513c, hsa-miR-589*, hsa-miR-26b*, hsa-miR-1200, hsa-miR-29b, hsa-miR-193a-3p, hsa-miR-186*, hsa-miR-520c-3p, hsa-miR-608, hsa-miR-1323, hsa-miR-384, hsa-miR-302a, hsa-miR-30d*, hsa-miR-30e*, hsa-miR-21 *, hsa-miR-29a, hsa-miR-503, hsa-miR-768-3p, hsa-miR-512-3p, hsa-miR-520d-3p, hsa-miR-519d, hsa-miR-452*, hsa-miR-935, hsa-miR-607, hsa-miR-520e, hsa-miR-578, hsa-miR-30d, hsa-miR-93, hsa-miR-193b, hsa-miR-152, hsa-miR-23b, hsa-miR-1322, hsa-miR-1257, hsa-miR-548a-3p, hsa-miR-301a, hsa-miR-526b*, hsa-miR-22*, hsa-miR-203, hsa-miR-625*, hsa-miR-597, hsa-miR-1185, hsa-miR-211, hsa-miR-593*, hsa-miR-1300, hsa-miR-150, hsa-miR-30c, hsa-miR-181a-2*, hsa-miR-583, hsa-miR-199b-5p, hsa-miR-205, hsa-miR-20b, hsa-miR-640, hsa-miR-106a, hsa-miR-1245, hsa-miR-138-2*, hsa-miR-877*, hsa-miR-149, hsa-miR-130a*, hsa-miR-520h, hsa-miR-431, hsa-miR-1228, hsa-miR-1827, hsa-miR-24, hsa-miR-548c-3p, hsa-miR-17, hsa-miR-382, hsa-miR-454, hsa-miR-5481, hsa-miR-654-5p, hsa-miR-302b, hsa-miR-1283, hsa-miR-548e, hsa-miR-21, hsa-miR-655, hsa-miR-888*, hsa-miR-206, hsa-miR-196a, hsa-miR-19a, hsa-miR-20a, hsa-miR-183, hsa-miR-455-3p, hsa-miR-196b, hsa-miR-126*, hsa-miR-1272, hsa-miR-767-5p, hsa-miR-499-5p, hsa-miR-106b, hsa-miR-369-3p, hsa-miR-372, hsa-miR-433, hsa-miR-541, hsa-miR-1302, hsa-miR-613, hsa-miR-584, hsa-miR-30e.

Coatomer protein complex, subunit beta 2 (beta prime)—COPB2—NM_{—}004766—hsa-miR-374b, hsa-miR-551b*, hsa-miR-330-3p, hsa-miR-128, hsa-miR-633, hsa-miR-654-3p, hsa-miR-570, hsa-miR-299-3p, hsa-miR-1256, hsa-miR-548c-3p, hsa-miR-374a.

Solute carrier family 25 (mitochondrial carrier; phosphate carrier) member 25—SLC25A25—Variant 1, Variant 2, Variant 3 and Variant 4—NM_{—}052901, NM_{—}001006641, NM_{—}001006642 and NM_{—}001007743—hsa-miR-576-3p, hsa-miR-194*, hsa-miR-132*, hsa-miR-453, hsa-miR-637, hsa-miR-509-3-5p, hsa-miR-490-5p, hsa-miR-642, hsa-miR-922, hsa-miR-1228*, hsa-miR-766, hsa-miR-1184, hsa-miR-125b-2*, hsa-miR-181 a, hsa-miR-519b-3p, hsa-miR-96, hsa-miR-604, hsa-miR-214, hsa-miR-532-3p, hsa-miR-549, hsa-miR-647, hsa-miR-27a, hsa-miR-92a-2*, hsa-miR-575, hsa-miR-661, hsa-miR-1237, hsa-miR-519c-3p, hsa-miR-129-5p, hsa-miR-93*, hsa-miR-886-3p, hsa-miR-128, hsa-miR-134, hsa-miR-519a, hsa-miR-298, hsa-miR-508-5p, hsa-miR-130b, hsa-miR-370, hsa-miR-1207-5p, hsa-miR-1182, hsa-miR-129*, hsa-miR-1246, hsa-miR-1203, hsa-miR-615-3p, hsa-miR-301b, hsa-miR-1236, hsa-miR-491-5p, hsa-miR-410, hsa-miR-421, hsa-miR-212, hsa-miR-92b*, hsa-miR-371-5p, hsa-miR-1, hsa-miR-1207-3p, hsa-miR-130a, hsa-miR-145, hsa-miR-220c, hsa-miR-1286, hsa-miR-99a*, hsa-miR-484, hsa-miR-1202, hsa-miR-1324, hsa-miR-1271, hsa-miR-29c, hsa-miR-132, hsa-miR-7-1*, hsa-miR-589*, hsa-miR-29b, hsa-miR-579, hsa-miR-1243, hsa-miR-873, hsa-miR-608, hsa-miR-185*, hsa-miR-505*, hsa-miR-596, hsa-miR-29a, hsa-miR-940, hsa-miR-1294, hsa-miR-184, hsa-miR-92a-1*, hsa-miR-181d, hsa-miR-504, hsa-miR-1204, hsa-miR-27b, hsa-miR-299-3p, hsa-miR-607, hsa-miR-1258, hsa-miR-518c*, hsa-miR-1260, hsa-miR-33b, hsa-miR-149*, hsa-miR-301a, hsa-miR-203, hsa-miR-361-3p, hsa-miR-628-5p, hsa-miR-150, hsa-miR-181c, hsa-miR-501-5p, hsa-miR-659, hsa-miR-378*, hsa-miR-1256, hsa-miR-1224-3p, hsa-miR-877*, hsa-miR-18a*, hsa-miR-30b*, hsa-miR-129-3p, hsa-miR-24, hsa-miR-1299, hsa-miR-454, hsa-miR-99b*, hsa-miR-181b, hsa-miR-206, hsa-miR-7-2*, hsa-miR-33a, hsa-miR-455-3p, hsa-miR-509-5p, hsa-miR-767-5p, hsa-miR-154, hsa-miR-613, hsa-miR-1225-3p.

EGFR-coamplified and overexpressed protein—ECOP—NM_{—}030796—hsa-miR-576-3p, hsa-miR-557, hsa-miR-665, hsa-miR-510, hsa-miR-34c-5p, hsa-miR-515-5p, hsa-miR-22, hsa-miR-648, hsa-miR-766, hsa-miR-1184, hsa-miR-520g, hsa-miR-302c*, hsa-miR-532-3p, hsa-miR-377, hsa-miR-27a, hsa-miR-1248, hsa-miR-335, hsa-miR-516a-3p, hsa-miR-511, hsa-miR-661, hsa-miR-486-3p, hsa-miR-335*, hsa-miR-507, hsa-miR-449a, hsa-miR-28-5p, hsa-miR-129-5p, hsa-miR-194, hsa-miR-217, hsa-miR-938, hsa-miR-342-3p, hsa-miR-298, hsa-miR-508-5p, hsa-miR-455-5p, hsa-miR-619, hsa-miR-587, hsa-miR-370, hsa-miR-103, hsa-miR-651, hsa-miR-580, hsa-miR-148a, hsa-miR-576-5p, hsa-miR-30b, hsa-miR-143*, hsa-miR-365, hsa-miR-449b, hsa-miR-1201, hsa-miR-606, hsa-miR-641, hsa-miR-212, hsa-miR-516b*, hsa-miR-329, hsa-miR-708, hsa-miR-146a*, hsa-miR-153, hsa-miR-145*, hsa-miR-494, hsa-miR-30a, hsa-miR-29a*, hsa-miR-622, hsa-miR-1, hsa-miR-1207-3p, hsa-miR-145, hsa-miR-874, hsa-miR-148b, hsa-miR-1286, hsa-miR-32*, hsa-miR-628-3p, hsa-miR-188-3p, hsa-miR-617, hsa-miR-495, hsa-miR-513c, hsa-miR-132, hsa-miR-7-1*, hsa-miR-34a, hsa-miR-361-5p, hsa-miR-374b*, hsa-miR-1233, hsa-miR-323-5p, hsa-miR-802, hsa-miR-570, hsa-miR-34b, hsa-miR-432, hsa-miR-768-3p, hsa-miR-34b*, hsa-miR-144*, hsa-miR-20a*, hsa-miR-27b, hsa-miR-636, hsa-miR-30d, hsa-miR-566, hsa-miR-139-3p, hsa-miR-152, hsa-miR-362-3p, hsa-miR-1185, hsa-miR-519e*, hsa-miR-30c, hsa-miR-760, hsa-miR-631, hsa-miR-1224-3p, hsa-miR-18a*, hsa-miR-30b*, hsa-miR-520h, hsa-miR-197, hsa-miR-1827, hsa-miR-24, hsa-miR-1299, hsa-miR-500*, hsa-miR-106a*, hsa-miR-7, hsa-miR-626, hsa-miR-206, hsa-miR-122, hsa-miR-7-2*, hsa-miR-107, hsa-miR-218, hsa-miR-595, hsa-miR-1234, hsa-miR-558, hsa-miR-1293, hsa-miR-613, hsa-miR-30e.

Phosphodiesterase 8A—PDE8A—Variant 1 and Variant 2—NM_{—}002605 and NM_{—}173454—hsa-miR-376b, hsa-miR-520b, hsa-miR-32, hsa-miR-513a-5p, hsa-miR-1184, hsa-miR-222*, hsa-miR-519b-3p, hsa-miR-182*, hsa-miR-302c*, hsa-miR-668, hsa-miR-214, hsa-miR-647, hsa-miR-1248, hsa-miR-520a-3p, hsa-miR-15b*, hsa-miR-31*, hsa-miR-513a-3p, hsa-miR-335*, hsa-miR-183*, hsa-miR-28-5p, hsa-miR-561, hsa-miR-519c-3p, hsa-miR-302e, hsa-miR-519a, hsa-miR-298, hsa-miR-508-5p, hsa-miR-92b, hsa-miR-455-5p, hsa-miR-1280, hsa-miR-1252, hsa-miR-367, hsa-miR-143*, hsa-miR-25, hsa-miR-302c, hsa-miR-548d-3p, hsa-miR-708, hsa-miR-146a*, hsa-miR-411, hsa-miR-376a, hsa-miR-373, hsa-miR-581, hsa-miR-145, hsa-miR-664, hsa-miR-573, hsa-miR-302d, hsa-miR-450a, hsa-miR-96*, hsa-miR-590-3p, hsa-miR-26b*, hsa-miR-936, hsa-miR-1233, hsa-miR-520c-3p, hsa-miR-185*, hsa-miR-144, hsa-miR-302a, hsa-miR-488, hsa-miR-512-3p, hsa-miR-520d-3p, hsa-miR-450b-5p, hsa-miR-452*, hsa-miR-607, hsa-miR-520e, hsa-miR-1260, hsa-miR-545*, hsa-miR-330-5p, hsa-miR-139-5p, hsa-miR-203, hsa-miR-92a, hsa-miR-432*, hsa-miR-760, hsa-miR-224, hsa-miR-1284, hsa-miR-1256, hsa-miR-199a-3p, hsa-miR-18a*, hsa-miR-522, hsa-miR-500*, hsa-miR-302b, hsa-miR-199b-3p, hsa-miR-1278, hsa-miR-577, hsa-miR-363, hsa-miR-219-2-3p, hsa-let-7f-2*, hsa-miR-372, hsa-miR-326, hsa-miR-497*, hsa-miR-142-3p, hsa-miR-1293.

Signal tranducing adaptor molecule (SH3 domain and ITAM motif) 1—STAM—NM_{—}003473—hsa-miR-32, hsa-miR-625, hsa-miR-493*, hsa-miR-138, hsa-miR-216a, hsa-miR-9, hsa-miR-1267, hsa-miR-1248, hsa-miR-575, hsa-miR-513a-3p, hsa-miR-544, hsa-miR-548m, hsa-miR-128, hsa-miR-92b, hsa-miR-663, hsa-miR-619, hsa-miR-299-5p, hsa-miR-367, hsa-miR-25, hsa-miR-186, hsa-miR-337-3p, hsa-miR-641, hsa-miR-146a*, hsa-miR-145*, hsa-miR-494, hsa-miR-145, hsa-miR-32*, hsa-miR-188-3p, hsa-miR-643, hsa-miR-579, hsa-miR-374b*, hsa-miR-144, hsa-miR-488, hsa-miR-588, hsa-miR-487b, hsa-miR-935, hsa-miR-548b-3p, hsa-miR-539, hsa-miR-203, hsa-miR-92a, hsa-miR-597, hsa-miR-1185, hsa-miR-205, hsa-miR-185, hsa-miR-631, hsa-miR-199a-3p, hsa-miR-197, hsa-miR-656, hsa-miR-199b-3p, hsa-miR-1244, hsa-miR-409-3p, hsa-miR-363, hsa-let-7f-2*, hsa-miR-649, hsa-miR-142-3p, hsa-miR-1205.

Tubulin, beta—TUBB_—NM_{—}178014—hsa-miR-124*, hsa-miR-200b, hsa-miR-1254, hsa-miR-642, hsa-miR-16, hsa-miR-515-5p, hsa-miR-625, hsa-miR-130b*, hsa-miR-216a, hsa-let-7d, hsa-miR-181a, hsa-miR-182*, hsa-miR-204, hsa-miR-92a-2*, hsa-miR-127-5p, hsa-miR-24-1*, hsa-miR-486-3p, hsa-miR-28-5p, hsa-miR-541*, hsa-miR-93*, hsa-miR-508-5p, hsa-miR-455-5p, hsa-let-7e, hsa-miR-623, hsa-miR-339-3p, hsa-miR-651, hsa-miR-548p, hsa-miR-876-5p, hsa-miR-208a, hsa-miR-491-5p, hsa-miR-520a-5p, hsa-miR-24-2*, hsa-miR-708, hsa-miR-525-5p, hsa-miR-371-5p, hsa-miR-548o, hsa-miR-200c, hsa-let-7f, hsa-miR-32*, hsa-miR-765, hsa-miR-15b, hsa-miR-495, hsa-miR-1208, hsa-miR-26b*, hsa-miR-605, hsa-miR-768-5p, hsa-miR-27a*, hsa-miR-1323, hsa-miR-888, hsa-miR-429, hsa-let-7g, hsa-miR-432, hsa-miR-1270, hsa-miR-769-3p, hsa-miR-450b-3p, hsa-miR-195, hsa-miR-545, hsa-miR-181d, hsa-miR-200a, hsa-let-7i, hsa-miR-23a*, hsa-miR-609, hsa-miR-23b*, hsa-miR-516b, hsa-let-7b, hsa-miR-620, hsa-miR-330-5p, hsa-miR-141, hsa-let-7a, hsa-miR-98, hsa-miR-571, hsa-miR-211, hsa-miR-519e*, hsa-miR-361-3p, hsa-miR-593*, hsa-miR-1300, hsa-miR-628-5p, hsa-miR-150, hsa-miR-181c, hsa-miR-760, hsa-miR-659, hsa-miR-1321, hsa-miR-208b, hsa-let-7c, hsa-miR-1827, hsa-miR-345, hsa-miR-424, hsa-miR-181b, hsa-miR-188-5p, hsa-miR-15a, hsa-miR-497, hsa-miR-369-3p, hsa-miR-326, hsa-miR-595, hsa-miR-1302.

Sorting nexin 6—SNX6—Variant 1 and Variant 2—NM_{—}021249 and NM_{—}152233—hsa-miR-642, hsa-miR-16, hsa-miR-515-5p, hsa-miR-542-3p, hsa-miR-922, hsa-miR-130b*, hsa-miR-219-1-3p, hsa-let-7d, hsa-miR-644, hsa-miR-302c*, hsa-miR-487a, hsa-miR-1179, hsa-miR-661, hsa-miR-375, hsa-miR-15b*, hsa-miR-31, hsa-miR-513a-3p, hsa-miR-548m, hsa-miR-330-3p, hsa-miR-191, hsa-let-7c*, hsa-miR-128, hsa-miR-1268, hsa-miR-619, hsa-let-7e, hsa-miR-424*, hsa-miR-1252, hsa-miR-630, hsa-miR-129*, hsa-miR-586, hsa-miR-25*, hsa-miR-30b, hsa-miR-1305, hsa-miR-33a*, hsa-miR-606, hsa-miR-520a-5p, hsa-miR-410, hsa-miR-421, hsa-miR-521, hsa-miR-525-5p, hsa-miR-30a, hsa-miR-874, hsa-miR-26b, hsa-let-7f, hsa-miR-15b, hsa-miR-495, hsa-miR-513c, hsa-miR-1297, hsa-miR-1208, hsa-miR-146a, hsa-miR-27a*, hsa-miR-509-3p, hsa-miR-624*, hsa-miR-570, hsa-miR-105, hsa-let-7g, hsa-miR-1294, hsa-miR-195, hsa-miR-30d, hsa-let-7i, hsa-miR-146b-5p, hsa-let-7b, hsa-miR-223, hsa-let-7a, hsa-miR-98, hsa-miR-519e*, hsa-miR-30c, hsa-miR-583, hsa-miR-631, hsa-miR-18a*, hsa-miR-522, hsa-let-7c, hsa-miR-129-3p, hsa-miR-1827, hsa-miR-548c-3p, hsa-miR-424, hsa-miR-655, hsa-miR-122, hsa-miR-15a, hsa-miR-338-3p, hsa-miR-944, hsa-miR-574-5p, hsa-miR-340, hsa-miR-497, hsa-miR-369-3p, hsa-miR-497*, hsa-miR-595, hsa-miR-499-3p, hsa-miR-26a, hsa-miR-154*, hsa-miR-512-5p, hsa-miR-30e.

RAB23, member RAS oncogene family—RAB23—Variant 1 and Variant 2—NM_{—}016277 and NM_{—}183227—hsa-miR-576-3p, hsa-miR-518d-5p, hsa-miR-200b, hsa-miR-509-3-5p, hsa-miR-520b, hsa-miR-32, hsa-miR-380, hsa-miR-16, hsa-miR-519a*, hsa-miR-522*, hsa-miR-493*, hsa-miR-513a-5p, hsa-miR-125b-2*, hsa-miR-374b, hsa-miR-195*, hsa-miR-221, hsa-miR-96, hsa-miR-1289, hsa-miR-1179, hsa-miR-30c-1*, hsa-miR-1826, hsa-miR-1248, hsa-miR-16-2*, hsa-miR-320c, hsa-miR-520a-3p, hsa-miR-31, hsa-miR-513a-3p, hsa-miR-183*, hsa-miR-330-3p, hsa-miR-28-5p, hsa-miR-302e, hsa-miR-93*, hsa-miR-508-5p, hsa-miR-92b, hsa-miR-1287, hsa-miR-875-3p, hsa-miR-299-5p, hsa-miR-548p, hsa-miR-586, hsa-miR-367, hsa-miR-338-5p, hsa-miR-30b, hsa-miR-25, hsa-miR-186, hsa-miR-30c-2*, hsa-miR-337-3p, hsa-miR-302c, hsa-miR-548d-3p, hsa-miR-329, hsa-let-7b*, hsa-miR-708, hsa-miR-30a*, hsa-miR-30a, hsa-miR-622, hsa-miR-373, hsa-miR-200c, hsa-miR-1207-3p, hsa-miR-548f, hsa-miR-220c, hsa-miR-573, hsa-miR-302d, hsa-miR-624, hsa-miR-15b, hsa-miR-320d, hsa-miR-590-3p, hsa-miR-1271, hsa-miR-579, hsa-miR-520c-3p, hsa-miR-770-5p, hsa-miR-1262, hsa-miR-144, hsa-miR-653, hsa-miR-302a, hsa-miR-30d*, hsa-miR-30e*, hsa-miR-429, hsa-miR-624*, hsa-miR-488, hsa-miR-222, hsa-miR-518f*, hsa-miR-768-3p, hsa-miR-520d-3p, hsa-miR-195, hsa-miR-331-5p, hsa-miR-450b-5p, hsa-miR-519d, hsa-miR-545, hsa-miR-299-3p, hsa-miR-323-3p, hsa-miR-607, hsa-miR-520e, hsa-miR-520c-5p, hsa-miR-30d, hsa-miR-10b*, hsa-miR-629, hsa-miR-93, hsa-miR-362-3p, hsa-miR-548a-3p, hsa-miR-1255a, hsa-miR-33b, hsa-miR-526b, hsa-miR-526b*, hsa-miR-92a, hsa-miR-518e*, hsa-let-7f-1*, hsa-miR-432*, hsa-miR-150, hsa-miR-30c, hsa-miR-526a, hsa-miR-1284, hsa-miR-20b, hsa-miR-378*, hsa-miR-640, hsa-miR-523*, hsa-miR-106a, hsa-miR-138-2*, hsa-miR-320a, hsa-miR-522, hsa-miR-197, hsa-miR-17, hsa-miR-106a*, hsa-miR-320b, hsa-miR-297, hsa-miR-5481, hsa-miR-424, hsa-miR-302b, hsa-miR-548e, hsa-miR-1251, hsa-miR-888*, hsa-miR-577, hsa-miR-33a, hsa-miR-15a, hsa-miR-374a, hsa-miR-20a, hsa-miR-409-3p, hsa-miR-363, hsa-miR-519b-5p, hsa-let-7a*, hsa-miR-338-3p, hsa-miR-509-5p, hsa-miR-499-5p, hsa-miR-106b, hsa-miR-497, hsa-miR-1197, hsa-miR-519c-5p, hsa-miR-16-1*, hsa-miR-649, hsa-miR-372, hsa-miR-154, hsa-miR-1255b, hsa-miR-30e.

Phospholipase A2-activating protein—PLAA—Variant 1—NM_{—}001031689—hsa-miR-642, hsa-miR-19b, hsa-miR-130b, hsa-miR-630, hsa-miR-148a, hsa-miR-374a*, hsa-miR-301b, hsa-miR-1236, hsa-miR-130a, hsa-miR-148b, hsa-miR-590-3p, hsa-miR-152, hsa-miR-301 a, hsa-miR-877*, hsa-miR-454, hsa-miR-19a.

PLAA—Variant 2—NM_{—}004253—hsa-miR-124*, hsa-miR-200b, hsa-miR-1254, hsa-miR-642, hsa-miR-493 *, hsa-miR-216a, hsa-miR-616, hsa-miR-101*, hsa-miR-551b*, hsa-miR-182*, hsa-miR-204, hsa-miR-376c, hsa-miR-27a, hsa-miR-138-1*, hsa-miR-26a-1*, hsa-miR-379*, hsa-miR-19b, hsa-miR-194, hsa-miR-541*, hsa-miR-892b, hsa-miR-769-5p, hsa-miR-130b, hsa-miR-630, hsa-miR-129*, hsa-miR-148a, hsa-miR-582-5p, hsa-miR-374a*, hsa-miR-301b, hsa-miR-1236, hsa-miR-590-5p, hsa-miR-548o, hsa-miR-200c, hsa-miR-130a, hsa-miR-616*, hsa-miR-148b, hsa-miR-373*, hsa-miR-590-3p, hsa-miR-7-1*, hsa-miR-411*, hsa-miR-26a-2*, hsa-miR-193a-3p, hsa-miR-1259, hsa-miR-1323, hsa-miR-802, hsa-miR-429, hsa-miR-769-3p, hsa-miR-450b-3p, hsa-miR-512-3p, hsa-miR-15a*, hsa-miR-27b, hsa-miR-607, hsa-miR-193b, hsa-miR-934, hsa-miR-152, hsa-miR-328, hsa-miR-301a, hsa-miR-203, hsa-miR-708*, hsa-miR-211, hsa-miR-877*, hsa-miR-129-3p, hsa-miR-454, hsa-miR-21, hsa-miR-655, hsa-miR-7-2*, hsa-miR-19a, hsa-miR-1244, hsa-miR-944, hsa-miR-193b*, hsa-miR-369-3p.

Stanniocalcin 2—STC2—NM_{—}003714—hsa-miR-140-5p, hsa-miR-124*, hsa-miR-665, hsa-miR-637, hsa-miR-200b, hsa-miR-509-3-5p, hsa-miR-490-5p, hsa-miR-448, hsa-miR-141*, hsa-miR-625, hsa-miR-219-1-3p, hsa-miR-1184, hsa-miR-133a, hsa-miR-222*, hsa-miR-181a, hsa-miR-519b-3p, hsa-miR-644, hsa-miR-125a-5p, hsa-miR-483-3p, hsa-miR-548g, hsa-miR-204, hsa-let-7g*, hsa-miR-96, hsa-miR-1289, hsa-miR-377, hsa-miR-376c, hsa-miR-647, hsa-miR-335, hsa-miR-92a-2*, hsa-miR-513a-3p, hsa-miR-29b-1*, hsa-miR-548m, hsa-miR-330-3p, hsa-miR-28-5p, hsa-miR-190, hsa-miR-519c-3p, hsa-miR-19b, hsa-miR-129-5p, hsa-miR-93*, hsa-miR-769-5p, hsa-miR-519a, hsa-miR-298, hsa-miR-508-5p, hsa-miR-1231, hsa-miR-663, hsa-miR-1303, hsa-miR-1287, hsa-miR-623, hsa-miR-875-3p, hsa-miR-651, hsa-miR-299-5p, hsa-miR-524-5p, hsa-miR-34a*, hsa-miR-33a*, hsa-miR-410, hsa-miR-421, hsa-miR-212, hsa-miR-329, hsa-let-7b*, hsa-miR-708, hsa-miR-146a*, hsa-miR-30a*, hsa-miR-489, hsa-miR-324-3p, hsa-miR-1, hsa-miR-200c, hsa-miR-1207-3p, hsa-miR-616*, hsa-miR-874, hsa-miR-373*, hsa-miR-532-5p, hsa-miR-1324, hsa-miR-617, hsa-miR-1271, hsa-miR-29c, hsa-miR-132, hsa-miR-26b*, hsa-miR-1291, hsa-miR-190b, hsa-miR-29b, hsa-miR-1243, hsa-miR-147, hsa-miR-873, hsa-miR-1226, hsa-miR-185*, hsa-miR-125b, hsa-miR-505*, hsa-miR-30d*, hsa-miR-30e*, hsa-miR-429, hsa-miR-29a, hsa-miR-570, hsa-miR-588, hsa-miR-1261, hsa-miR-1270, hsa-miR-769-3p, hsa-miR-34b*, hsa-miR-148b*, hsa-miR-450b-3p, hsa-miR-20a*, hsa-miR-181d, hsa-miR-504, hsa-miR-1204, hsa-miR-607, hsa-miR-629*, hsa-miR-383, hsa-miR-29b-2*, hsa-miR-192*, hsa-miR-942, hsa-miR-101, hsa-miR-362-3p, hsa-miR-545*, hsa-miR-1229, hsa-miR-133b, hsa-miR-620, hsa-miR-328, hsa-miR-330-5p, hsa-miR-941, hsa-miR-625*, hsa-miR-1185, hsa-miR-744*, hsa-miR-211, hsa-miR-361-3p, hsa-miR-593*, hsa-let-7f-1*, hsa-miR-1238, hsa-miR-628-5p, hsa-miR-181c, hsa-miR-181a-2*, hsa-miR-659, hsa-miR-138-2*, hsa-miR-483-5p, hsa-miR-550, hsa-miR-1228, hsa-miR-24, hsa-miR-1299, hsa-miR-548c-3p, hsa-miR-663b, hsa-miR-106a*, hsa-miR-656, hsa-miR-345, hsa-miR-548k, hsa-miR-181b, hsa-miR-891a, hsa-miR-485-5p, hsa-miR-137, hsa-miR-1278, hsa-miR-892a, hsa-miR-206, hsa-miR-19a, hsa-miR-1244, hsa-miR-425, hsa-miR-455-3p, hsa-let-7a*, hsa-miR-509-5p, hsa-miR-340, hsa-miR-193b*, hsa-miR-367*, hsa-miR-326, hsa-miR-223*, hsa-miR-520d-5p, hsa-miR-1205, hsa-miR-613, hsa-miR-512-5p.

Lactotransferrin—LTF—NM_{—}002343—hsa-miR-637, hsa-miR-34c-5p, hsa-miR-214, hsa-miR-920, hsa-miR-513a-3p, hsa-miR-486-3p, hsa-miR-449a, hsa-miR-208a, hsa-miR-449b, hsa-miR-329, hsa-miR-939, hsa-miR-1253, hsa-miR-34a, hsa-miR-362-3p, hsa-miR-1257, hsa-miR-432*, hsa-miR-185, hsa-miR-1224-3p, hsa-miR-208b, hsa-miR-1228, hsa-miR-219-2-3p.

Interferon regulator factor 9—IRF9—NM_{—}006084—hsa-miR-520b, hsa-miR-130b*, hsa-miR-1290, hsa-miR-549, hsa-miR-138-1*, hsa-miR-575, hsa-miR-520a-3p, hsa-miR-585, hsa-miR-142-5p, hsa-miR-302e, hsa-miR-199a-5p, hsa-miR-580, hsa-miR-600, hsa-miR-302c, hsa-miR-606, hsa-miR-373, hsa-miR-616*, hsa-miR-373*, hsa-miR-302d, hsa-miR-590-3p, hsa-miR-520f, hsa-miR-520c-3p, hsa-miR-1226, hsa-miR-384, hsa-miR-302a, hsa-miR-1294, hsa-miR-520d-3p, hsa-miR-519d, hsa-miR-636, hsa-miR-520e, hsa-miR-93, hsa-miR-934, hsa-miR-554, hsa-miR-526b*, hsa-miR-571, hsa-miR-361-3p, hsa-miR-583, hsa-miR-199b-5p, hsa-miR-20b, hsa-miR-106a, hsa-miR-30b*, hsa-miR-17, hsa-miR-302b, hsa-miR-331-3p, hsa-miR-20a, hsa-miR-627, hsa-miR-340, hsa-miR-106b, hsa-miR-1197, hsa-miR-372.

Ataxin 3—ATXN3—Variant 1 and Variant 2—NM_{—}004993 and NM_{—}030660—hsa-miR-576-3p, hsa-miR-124*, hsa-miR-665, hsa-miR-520b, hsa-miR-32, hsa-miR-645, hsa-miR-448, hsa-miR-922, hsa-miR-493*, hsa-miR-130b*, hsa-miR-766, hsa-miR-1276, hsa-miR-1184, hsa-miR-520g, hsa-miR-616, hsa-miR-1290, hsa-miR-181a, hsa-miR-551b*, hsa-miR-195*, hsa-miR-302c*, hsa-miR-487a, hsa-let-7g*, hsa-miR-96, hsa-miR-377, hsa-miR-30c-1*, hsa-miR-890, hsa-miR-376c, hsa-miR-27a, hsa-miR-516a-3p, hsa-miR-16-2*, hsa-miR-320c, hsa-miR-520a-3p, hsa-miR-511, hsa-miR-661, hsa-miR-513a-3p, hsa-miR-26a-1*, hsa-miR-142-5p, hsa-miR-379*, hsa-miR-335*, hsa-miR-29b-1*, hsa-miR-548m, hsa-miR-302a*, hsa-miR-561, hsa-miR-1178, hsa-miR-217, hsa-miR-885-5p, hsa-miR-302e, hsa-miR-603, hsa-miR-128, hsa-miR-342-3p, hsa-miR-508-5p, hsa-miR-92b, hsa-miR-130b, hsa-miR-199a-5p, hsa-miR-623, hsa-miR-129*, hsa-miR-148a, hsa-miR-299-5p, hsa-miR-548p, hsa-miR-367, hsa-miR-582-5p, hsa-miR-338-5p, hsa-miR-524-5p, hsa-miR-543, hsa-miR-25, hsa-miR-186, hsa-miR-23a, hsa-miR-365, hsa-miR-301b, hsa-miR-30c-2*, hsa-miR-618, hsa-miR-33 a*, hsa-miR-302c, hsa-miR-302f, hsa-miR-641, hsa-miR-212, hsa-miR-516b*, hsa-miR-329, hsa-miR-590-5p, hsa-miR-494, hsa-miR-582-3p, hsa-miR-30a*, hsa-miR-371-5p, hsa-miR-489, hsa-miR-29a*, hsa-miR-622, hsa-miR-324-3p, hsa-miR-548o, hsa-miR-373, hsa-miR-130a, hsa-miR-616*, hsa-miR-552, hsa-miR-148b, hsa-miR-373*, hsa-miR-548f, hsa-miR-1286, hsa-miR-26b, hsa-miR-573, hsa-miR-32*, hsa-miR-302d, hsa-miR-1324, hsa-miR-320d, hsa-miR-617, hsa-miR-590-3p, hsa-miR-495, hsa-miR-1271, hsa-miR-29c, hsa-miR-513c, hsa-miR-132, hsa-miR-1297, hsa-miR-7-1*, hsa-miR-589*, hsa-miR-1253, hsa-miR-1200, hsa-miR-921, hsa-miR-643, hsa-miR-29b, hsa-miR-411*, hsa-miR-26a-2*, hsa-miR-193a-3p, hsa-miR-520f, hsa-miR-146a, hsa-miR-1259, hsa-miR-374b*, hsa-miR-186*, hsa-miR-1233, hsa-miR-520c-3p, hsa-miR-27a*, hsa-miR-498, hsa-miR-144, hsa-miR-1323, hsa-miR-220b, hsa-miR-302a, hsa-miR-888, hsa-miR-30d*, hsa-miR-568, hsa-miR-30e*, hsa-miR-29a, hsa-miR-488, hsa-miR-570, hsa-miR-940, hsa-miR-125a-3p, hsa-miR-1285, hsa-miR-520d-3p, hsa-miR-18b*, hsa-miR-450b-5p, hsa-miR-610, hsa-miR-545, hsa-miR-181d, hsa-miR-612, hsa-miR-9*, hsa-miR-514, hsa-miR-27b, hsa-miR-323-3p, hsa-miR-607, hsa-miR-520e, hsa-miR-200a, hsa-miR-578, hsa-miR-629, hsa-miR-383, hsa-miR-155*, hsa-miR-192*, hsa-miR-193b, hsa-miR-566, hsa-miR-518c*, hsa-miR-491-3p, hsa-miR-942, hsa-miR-152, hsa-miR-23b, hsa-miR-1322, hsa-miR-362-3p, hsa-miR-545*, hsa-miR-548a-3p, hsa-miR-146b-5p, hsa-miR-516b, hsa-miR-526b, hsa-miR-567, hsa-miR-302d*, hsa-miR-301a, hsa-miR-141, hsa-miR-92a, hsa-miR-625*, hsa-miR-744*, hsa-miR-593*, hsa-miR-628-5p, hsa-miR-150, hsa-miR-181c, hsa-miR-760, hsa-miR-501-5p, hsa-miR-199b-5p, hsa-miR-378*, hsa-miR-640, hsa-miR-1224-3p, hsa-miR-877*, hsa-miR-18a*, hsa-miR-525-3p, hsa-miR-30b*, hsa-miR-130a*, hsa-miR-550, hsa-miR-520h, hsa-miR-320a, hsa-miR-1225-5p, hsa-miR-197, hsa-miR-129-3p, hsa-miR-1827, hsa-miR-548c-3p, hsa-miR-135a*, hsa-miR-663b, hsa-miR-500*, hsa-miR-1279, hsa-miR-106a*, hsa-miR-382, hsa-miR-656, hsa-miR-320b, hsa-miR-454, hsa-miR-1264, hsa-miR-297, hsa-miR-5481, hsa-miR-654-5p, hsa-miR-302b, hsa-miR-181b, hsa-miR-548e, hsa-miR-1251, hsa-miR-21, hsa-miR-655, hsa-miR-485-5p, hsa-miR-614, hsa-miR-1278, hsa-miR-7-2*, hsa-miR-377*, hsa-miR-188-5p, hsa-miR-485-3p, hsa-miR-632, hsa-miR-409-3p, hsa-miR-183, hsa-miR-876-3p, hsa-miR-363, hsa-miR-1272, hsa-miR-767-5p, hsa-miR-944, hsa-miR-340, hsa-miR-499-5p, hsa-miR-193b*, hsa-miR-219-2-3p, hsa-miR-524-3p, hsa-let-7f-2*, hsa-miR-302b*, hsa-miR-372, hsa-miR-595, hsa-miR-26a, hsa-miR-541, hsa-miR-520d-5p, hsa-miR-1205, hsa-miR-154*, hsa-miR-671-5p, hsa-miR-340*, hsa-miR-584.

General transcription factor IIIC, polypeptide 3, 102 kDa—GTF3C3—NM_{—}012086—hsa-miR-448, hsa-miR-22, hsa-miR-125a-5p, hsa-miR-377, hsa-miR-452, hsa-miR-342-3p, hsa-miR-646, hsa-miR-1208, hsa-miR-26b*, hsa-miR-125b, hsa-miR-203, hsa-miR-522, hsa-miR-433, hsa-miR-1250.

Glycogen synthase kinase 3 beta—GSK3B—NM_{—}002093—hsa-miR-1301, hsa-miR-647, hsa-miR-199a-5p, hsa-miR-582-3p, hsa-miR-1207-3p, hsa-miR-26b, hsa-miR-1297, hsa-miR-199b-5p, hsa-miR-876-3p, hsa-miR-26a.

Kruppel-like factor 10—KLF10—Variant 1—NM_{—}005655—hsa-miR-200b, hsa-miR-509-3-5p, hsa-miR-19b-2*, hsa-miR-520g, hsa-miR-1290, hsa-miR-101*, hsa-miR-519b-3p, hsa-miR-513b, hsa-miR-195*, hsa-miR-548a-5p, hsa-miR-559, hsa-miR-1248, hsa-miR-548b-5p, hsa-miR-16-2*, hsa-miR-31*, hsa-miR-142-5p, hsa-miR-548m, hsa-miR-330-3p, hsa-miR-519c-3p, hsa-miR-19b, hsa-miR-541*, hsa-miR-548n, hsa-miR-892b, hsa-miR-548c-5p, hsa-miR-19a*, hsa-miR-1825, hsa-miR-519a, hsa-miR-663, hsa-miR-875-3p, hsa-miR-129*, hsa-miR-576-5p, hsa-miR-548p, hsa-miR-338-5p, hsa-miR-19b-1*, hsa-miR-30b, hsa-miR-143*, hsa-miR-23a, hsa-miR-618, hsa-miR-337-3p, hsa-miR-33a*, hsa-miR-548i, hsa-miR-410, hsa-miR-421, hsa-let-7b*, hsa-miR-30a, hsa-miR-200c, hsa-miR-26b, hsa-miR-513c, hsa-miR-1297, hsa-miR-7-1*, hsa-miR-34c-3p, hsa-miR-579, hsa-miR-520f, hsa-miR-802, hsa-miR-654-3p, hsa-miR-429, hsa-miR-570, hsa-miR-517*, hsa-miR-548d-5p, hsa-miR-34b, hsa-miR-148b*, hsa-miR-519d, hsa-miR-545, hsa-miR-452*, hsa-miR-548j, hsa-miR-607, hsa-miR-30d, hsa-miR-10b*, hsa-miR-93, hsa-miR-23b, hsa-miR-539, hsa-miR-1257, hsa-miR-889, hsa-miR-526b*, hsa-miR-203, hsa-miR-548h, hsa-let-7f-1 *, hsa-miR-30c, hsa-miR-224, hsa-miR-20b, hsa-miR-106a, hsa-miR-149, hsa-miR-130a*, hsa-miR-520h, hsa-miR-197, hsa-miR-129-3p, hsa-miR-17, hsa-miR-345, hsa-miR-548k, hsa-miR-655, hsa-miR-892a, hsa-miR-7-2*, hsa-miR-19a, hsa-miR-188-5p, hsa-miR-20a, hsa-let-7a*, hsa-miR-509-5p, hsa-miR-340, hsa-miR-106b, hsa-miR-369-3p, hsa-let-7f-2*, hsa-miR-499-3p, hsa-miR-662, hsa-miR-26a, hsa-miR-1205, hsa-miR-744, hsa-miR-30e.

KLF10—Variant 2—NM_{—}001032282—hsa-miR-200b, hsa-miR-509-3-5p, hsa-miR-19b-2*, hsa-miR-520g, hsa-miR-1290, hsa-miR-101*, hsa-miR-519b-3p, hsa-miR-513b, hsa-miR-195*, hsa-miR-548a-5p, hsa-miR-559, hsa-miR-1248, hsa-miR-548b-5p, hsa-miR-16-2*, hsa-miR-31*, hsa-miR-142-5p, hsa-miR-548m, hsa-miR-330-3p, hsa-miR-519c-3p, hsa-miR-19b, hsa-miR-541*, hsa-miR-548n, hsa-miR-892b, hsa-miR-548c-5p, hsa-miR-19a*, hsa-miR-1825, hsa-miR-519a, hsa-miR-875-3p, hsa-miR-129*, hsa-miR-576-5p, hsa-miR-548p, hsa-miR-338-5p, hsa-miR-19b-1*, hsa-miR-30b, hsa-miR-143*, hsa-miR-23a, hsa-miR-618, hsa-miR-337-3p, hsa-miR-33a*, hsa-miR-548i, hsa-miR-410, hsa-miR-421, hsa-let-7b*, hsa-miR-30a, hsa-miR-200c, hsa-miR-26b, hsa-miR-513c, hsa-miR-1297, hsa-miR-7-1*, hsa-miR-34c-3p, hsa-miR-579, hsa-miR-520f, hsa-miR-802, hsa-miR-654-3p, hsa-miR-429, hsa-miR-570, hsa-miR-517*, hsa-miR-548d-5p, hsa-miR-34b, hsa-miR-148b*, hsa-miR-519d, hsa-miR-545, hsa-miR-452*, hsa-miR-548j, hsa-miR-607, hsa-miR-30d, hsa-miR-10b*, hsa-miR-93, hsa-miR-23b, hsa-miR-539, hsa-miR-1257, hsa-miR-889, hsa-miR-526b*, hsa-miR-203, hsa-miR-548h, hsa-let-7f-1*, hsa-miR-30c, hsa-miR-224, hsa-miR-20b, hsa-miR-106a, hsa-miR-149, hsa-miR-130a*, hsa-miR-483-5p, hsa-miR-520h, hsa-miR-197, hsa-miR-129-3p, hsa-miR-17, hsa-miR-345, hsa-miR-548k, hsa-miR-655, hsa-miR-892a, hsa-miR-7-2*, hsa-miR-19a, hsa-miR-188-5p, hsa-miR-20a, hsa-let-7a*, hsa-miR-509-5p, hsa-miR-340, hsa-miR-106b, hsa-miR-369-3p, hsa-let-7f-2*, hsa-miR-499-3p, hsa-miR-662, hsa-miR-26a, hsa-miR-1205, hsa-miR-30e.

Elongation factor, RNA polymerase II,2—ELL2—NM_{—}012081—hsa-miR-576-3p, hsa-miR-557, hsa-miR-518d-5p, hsa-miR-510, hsa-miR-200b, hsa-miR-520b, hsa-miR-34c-5p, hsa-miR-16, hsa-miR-448, hsa-miR-519a*, hsa-miR-500, hsa-miR-20b*, hsa-miR-141*, hsa-miR-542-3p, hsa-miR-522*, hsa-miR-219-1-3p, hsa-miR-562, hsa-miR-875-5p, hsa-miR-766, hsa-miR-1276, hsa-miR-125b-2*, hsa-miR-520g, hsa-miR-551b*, hsa-miR-302c*, hsa-miR-221, hsa-miR-548g, hsa-miR-204, hsa-let-7g*, hsa-miR-28-3p, hsa-miR-890, hsa-miR-548a-5p, hsa-miR-376c, hsa-miR-490-3p, hsa-miR-559, hsa-miR-27a, hsa-miR-92a-2*, hsa-miR-548b-5p, hsa-miR-320c, hsa-miR-520a-3p, hsa-miR-513a-3p, hsa-miR-24-1*, hsa-miR-335*, hsa-miR-507, hsa-miR-449a, hsa-miR-29b-1*, hsa-miR-28-5p, hsa-miR-561, hsa-miR-19b, hsa-miR-129-5p, hsa-miR-541*, hsa-miR-548n, hsa-miR-892b, hsa-miR-885-5p, hsa-miR-302e, hsa-miR-93*, hsa-miR-548c-5p, hsa-miR-603, hsa-miR-574-3p, hsa-miR-409-5p, hsa-miR-130b, hsa-miR-619, hsa-miR-370, hsa-miR-1287, hsa-miR-299-5p, hsa-miR-576-5p, hsa-miR-876-5p, hsa-miR-30b, hsa-miR-186, hsa-miR-301b, hsa-miR-449b, hsa-miR-1305, hsa-miR-337-3p, hsa-miR-33a*, hsa-miR-302c, hsa-miR-491-5p, hsa-miR-548i, hsa-miR-218-2*, hsa-miR-24-2*, hsa-miR-646, hsa-let-7b*, hsa-miR-708, hsa-miR-590-5p, hsa-miR-30a*, hsa-miR-30a, hsa-miR-29a*, hsa-miR-548o, hsa-miR-373, hsa-miR-200c, hsa-miR-130a, hsa-miR-145, hsa-miR-616*, hsa-miR-373*, hsa-miR-548f, hsa-miR-569, hsa-miR-664, hsa-miR-1269, hsa-miR-302d, hsa-miR-624, hsa-miR-15b, hsa-miR-320d, hsa-miR-188-3p, hsa-miR-590-3p, hsa-miR-495, hsa-miR-29c, hsa-miR-513c, hsa-miR-635, hsa-miR-34a, hsa-miR-29b, hsa-miR-579, hsa-miR-1243, hsa-miR-520c-3p, hsa-miR-518a-5p, hsa-miR-608, hsa-miR-770-5p, hsa-miR-144, hsa-miR-1323, hsa-miR-220b, hsa-miR-302a, hsa-miR-30d*, hsa-miR-802, hsa-miR-30e*, hsa-miR-21*, hsa-miR-429, hsa-miR-29a, hsa-miR-508-3p, hsa-miR-155, hsa-miR-548d-5p, hsa-miR-588, hsa-miR-222, hsa-miR-518f*, hsa-miR-34b*, hsa-miR-125a-3p, hsa-miR-512-3p, hsa-miR-520d-3p, hsa-miR-195, hsa-miR-450b-5p, hsa-miR-519d, hsa-miR-9*, hsa-miR-27b, hsa-miR-664*, hsa-miR-548j, hsa-miR-323-3p, hsa-miR-607, hsa-miR-520e, hsa-miR-520c-5p, hsa-miR-30d, hsa-miR-10b*, hsa-miR-93, hsa-miR-383, hsa-miR-1322, hsa-miR-548a-3p, hsa-miR-567, hsa-miR-139-5p, hsa-miR-301a, hsa-miR-223, hsa-miR-526b*, hsa-miR-203, hsa-miR-571, hsa-miR-548h, hsa-miR-625*, hsa-miR-708*, hsa-miR-211, hsa-miR-518e*, hsa-miR-593*, hsa-let-7f-1*, hsa-miR-628-5p, hsa-miR-30c, hsa-miR-526a, hsa-miR-205, hsa-miR-224, hsa-miR-1284, hsa-miR-221*, hsa-miR-527, hsa-miR-20b, hsa-miR-640, hsa-miR-523*, hsa-miR-106a, hsa-miR-520h, hsa-miR-320a, hsa-miR-522, hsa-miR-555, hsa-miR-431, hsa-miR-548c-3p, hsa-miR-17, hsa-miR-500*, hsa-miR-106a*, hsa-miR-656, hsa-miR-182, hsa-miR-320b, hsa-miR-454, hsa-miR-1264, hsa-miR-297, hsa-miR-5481, hsa-miR-424, hsa-miR-302b, hsa-miR-548e, hsa-miR-21, hsa-miR-888*, hsa-miR-577, hsa-miR-19a, hsa-miR-15a, hsa-miR-20a, hsa-miR-519b-5p, hsa-let-7a*, hsa-miR-944, hsa-miR-340, hsa-miR-1265, hsa-miR-140-3p, hsa-miR-106b, hsa-miR-497, hsa-miR-519c-5p, hsa-miR-649, hsa-miR-372, hsa-miR-1183, hsa-miR-505, hsa-miR-595, hsa-miR-1293, hsa-miR-584, hsa-miR-30e.

Zinc finger and BTB domain containing 20—ZBTB20—NM_{—}015642—hsa-miR-380, hsa-miR-214, hsa-miR-548a-5p, hsa-miR-559, hsa-miR-548b-5p, hsa-miR-548c-5p, hsa-miR-548i, hsa-miR-548d-5p, hsa-miR-548j, hsa-miR-634, hsa-miR-22*, hsa-miR-548h, hsa-miR-296-3p.

Iroquois homeobox 3—IRX3—NM_{—}024336—hsa-miR-493*, hsa-miR-19b-2*, hsa-miR-520g, hsa-miR-551b*, hsa-miR-377, hsa-miR-29b-1*, hsa-miR-561, hsa-miR-19a*, hsa-miR-19b-1*, hsa-miR-646, hsa-let-7b*, hsa-miR-1227, hsa-miR-495, hsa-miR-331-5p, hsa-miR-149*, hsa-let-7f-1*, hsa-miR-342-5p, hsa-miR-1284, hsa-miR-659, hsa-miR-520h, hsa-miR-382, hsa-miR-125b-1*, hsa-miR-126*, hsa-let-7a*, hsa-miR-944, hsa-let-7f-2*.

v-ets erythroblastosis virus E26 oncogene homolog 1—ETS1—NM_{—}005238—hsa-miR-124*, hsa-miR-200b, hsa-miR-509-3-5p, hsa-miR-490-5p, hsa-miR-642, hsa-miR-431*, hsa-miR-1180, hsa-miR-515-5p, hsa-miR-27b*, hsa-miR-141*, hsa-miR-542-3p, hsa-miR-187, hsa-miR-922, hsa-miR-625, hsa-miR-130b*, hsa-miR-216a, hsa-miR-19b-2*, hsa-miR-766, hsa-miR-650, hsa-miR-125b-2*, hsa-miR-520g, hsa-miR-181a, hsa-miR-182*, hsa-miR-125a-5p, hsa-miR-221, hsa-miR-214, hsa-miR-532-3p, hsa-miR-377, hsa-miR-218-1*, hsa-miR-1248, hsa-miR-320c, hsa-miR-335*, hsa-miR-19b, hsa-miR-129-5p, hsa-miR-548n, hsa-miR-658, hsa-miR-19a*, hsa-miR-1825, hsa-miR-455-5p, hsa-miR-587, hsa-miR-199a-5p, hsa-miR-1303, hsa-miR-875-3p, hsa-miR-657, hsa-miR-630, hsa-miR-660, hsa-miR-148a, hsa-miR-576-5p, hsa-miR-548p, hsa-miR-586, hsa-miR-338-5p, hsa-miR-19b-1*, hsa-miR-25*, hsa-miR-543, hsa-miR-34a*, hsa-miR-208a, hsa-miR-23a, hsa-miR-365, hsa-miR-1236, hsa-miR-1305, hsa-miR-10a, hsa-miR-218-2*, hsa-miR-410, hsa-miR-421, hsa-miR-641, hsa-miR-646, hsa-miR-329, hsa-miR-146a*, hsa-miR-30a*, hsa-miR-489, hsa-miR-29a*, hsa-miR-548o, hsa-miR-1, hsa-miR-200c, hsa-miR-1207-3p, hsa-miR-148b, hsa-miR-569, hsa-miR-664, hsa-miR-1202, hsa-miR-765, hsa-miR-320d, hsa-miR-590-3p, hsa-miR-495, hsa-miR-513c, hsa-miR-7-1*, hsa-miR-1291, hsa-miR-605, hsa-miR-1259, hsa-miR-498, hsa-miR-144, hsa-miR-1323, hsa-miR-125b, hsa-miR-30d*, hsa-miR-654-3p, hsa-miR-568, hsa-miR-30e*, hsa-miR-429, hsa-miR-570, hsa-miR-124, hsa-miR-940, hsa-miR-155, hsa-miR-324-5p, hsa-miR-222, hsa-miR-769-3p, hsa-miR-593, hsa-miR-34b*, hsa-miR-148b*, hsa-miR-1285, hsa-miR-450b-3p, hsa-miR-545, hsa-miR-181d, hsa-miR-612, hsa-miR-607, hsa-miR-10b*, hsa-miR-629*, hsa-miR-548b-3p, hsa-miR-1260, hsa-miR-942, hsa-miR-152, hsa-miR-23b, hsa-miR-362-3p, hsa-miR-1229, hsa-miR-634, hsa-miR-136, hsa-miR-330-5p, hsa-miR-139-5p, hsa-miR-941, hsa-miR-203, hsa-miR-571, hsa-miR-492, hsa-miR-625*, hsa-miR-381, hsa-miR-708*, hsa-miR-519e*, hsa-miR-1300, hsa-miR-181c, hsa-miR-181 a-2*, hsa-miR-501-5p, hsa-miR-199b-5p, hsa-miR-342-5p, hsa-miR-493, hsa-miR-224, hsa-miR-221*, hsa-miR-17*, hsa-miR-1224-3p, hsa-miR-208b, hsa-miR-130a*, hsa-miR-758, hsa-miR-520h, hsa-miR-320a, hsa-miR-10b, hsa-miR-522, hsa-miR-486-5p, hsa-miR-1228, hsa-miR-1827, hsa-miR-1299, hsa-miR-548c-3p, hsa-miR-369-5p, hsa-miR-320b, hsa-miR-5481, hsa-miR-506, hsa-miR-626, hsa-miR-181b, hsa-miR-1283, hsa-miR-655, hsa-miR-206, hsa-miR-7-2*, hsa-miR-19a, hsa-miR-300, hsa-miR-632, hsa-miR-1244, hsa-miR-338-3p, hsa-miR-509-5p, hsa-miR-369-3p, hsa-miR-16-1*, hsa-miR-326, hsa-miR-497*, hsa-miR-223*, hsa-miR-1275, hsa-miR-1205, hsa-miR-135b*, hsa-miR-613, hsa-miR-512-5p, hsa-miR-584.

SERTA domain containing 1—SERTAD1—NM_{—}013376—hsa-miR-105*, hsa-miR-515-5p, hsa-miR-133a, hsa-miR-520g, hsa-miR-125a-5p, hsa-miR-28-3p, hsa-miR-214, hsa-miR-511, hsa-miR-892b, hsa-miR-146b-3p, hsa-miR-1252, hsa-miR-1236, hsa-miR-10a, hsa-miR-484, hsa-miR-1202, hsa-miR-34c-3p, hsa-miR-26b*, hsa-miR-361-5p, hsa-miR-376a*, hsa-miR-125b, hsa-miR-184, hsa-miR-504, hsa-miR-133b, hsa-miR-519e*, hsa-miR-611, hsa-miR-520h, hsa-miR-10b, hsa-miR-339-5p, hsa-miR-24, hsa-miR-548c-3p, hsa-miR-663b, hsa-miR-1274b, hsa-miR-671-5p.

Trans-membrane protein 101—TMEM101—NM_{—}032376—hsa-miR-194*, hsa-miR-509-3-5p, hsa-miR-1266, hsa-miR-642, hsa-miR-518e, hsa-miR-20b*, hsa-miR-515-5p, hsa-miR-141*, hsa-miR-542-3p, hsa-miR-22, hsa-miR-222*, hsa-miR-125a-5p, hsa-let-7g*, hsa-miR-214, hsa-miR-532-3p, hsa-miR-27a, hsa-miR-452, hsa-miR-320c, hsa-miR-1178, hsa-miR-603, hsa-miR-1207-5p, hsa-miR-1182, hsa-miR-548p, hsa-miR-23a, hsa-miR-33a*, hsa-miR-1201, hsa-miR-548d-3p, hsa-miR-646, hsa-miR-324-3p, hsa-miR-1207-3p, hsa-miR-320d, hsa-miR-590-3p, hsa-miR-495, hsa-miR-513c, hsa-miR-589, hsa-miR-589*, hsa-miR-768-5p, hsa-miR-147, hsa-miR-125b, hsa-miR-592, hsa-miR-940, hsa-miR-324-5p, hsa-miR-588, hsa-miR-27b, hsa-miR-23b, hsa-miR-1274a, hsa-miR-149*, hsa-miR-519e*, hsa-miR-150, hsa-miR-583, hsa-miR-221*, hsa-miR-556-5p, hsa-miR-640, hsa-miR-18a*, hsa-miR-30b*, hsa-miR-130a*, hsa-miR-320a, hsa-miR-486-5p, hsa-miR-320b, hsa-miR-331-3p, hsa-miR-892a, hsa-miR-509-5p, hsa-miR-649, hsa-miR-505, hsa-miR-558.

v-maf musculo-aponeurotic fibrosarcoma oncogene homolog F—MAFF—Variant 1 and Variant 2—NM_{—}012323 and NM_{—}152878—hsa-miR-576-3p, hsa-miR-194*, hsa-miR-665, hsa-miR-510, hsa-miR-509-3-5p, hsa-miR-520b, hsa-miR-1266, hsa-miR-34c-5p, hsa-miR-642, hsa-miR-20b*, hsa-miR-562, hsa-miR-133a, hsa-miR-520g, hsa-miR-1290, hsa-miR-195*, hsa-miR-96, hsa-miR-214, hsa-miR-532-3p, hsa-miR-647, hsa-miR-1248, hsa-miR-920, hsa-miR-92a-2*, hsa-miR-16-2*, hsa-miR-520a-3p, hsa-miR-486-3p, hsa-miR-449a, hsa-miR-378, hsa-miR-28-5p, has-miR-302a*, hsa-miR-1237, hsa-miR-1288, hsa-miR-302e, hsa-miR-1825, hsa-miR-128, hsa-miR-1268, hsa-miR-508-5p, hsa-miR-130b, hsa-miR-663, hsa-miR-1207-5p, hsa-miR-623, hsa-miR-1182, hsa-miR-148a, hsa-miR-143*, hsa-miR-186, hsa-miR-301b, hsa-miR-449b, hsa-miR-1236, hsa-miR-302c, hsa-miR-491-5p, hsa-miR-708, hsa-miR-371-5p, hsa-miR-324-3p, hsa-miR-1, hsa-miR-373, hsa-miR-130a, hsa-miR-148b, hsa-miR-1286, hsa-miR-302d, hsa-miR-1324, hsa-miR-1271, hsa-miR-26b*, hsa-miR-34a, hsa-miR-193a-3p, hsa-miR-768-5p, hsa-miR-186*, hsa-miR-520c-3p, hsa-miR-27a*, hsa-miR-185*, hsa-miR-302a, hsa-miR-1224-5p, hsa-miR-940, hsa-miR-1285, hsa-miR-512-3p, hsa-miR-520d-3p, hsa-miR-519d, hsa-miR-612, hsa-miR-520e, hsa-miR-629*, hsa-miR-93, hsa-miR-193b, hsa-miR-1260, hsa-miR-942, hsa-miR-152, hsa-miR-422a, hsa-miR-1274a, hsa-miR-539, hsa-miR-133b, hsa-miR-149*, hsa-miR-301a, hsa-miR-526b*, hsa-miR-203, hsa-miR-492, hsa-miR-597, hsa-miR-744*, hsa-miR-361-3p, hsa-miR-150, hsa-miR-760, hsa-miR-342-5p, hsa-miR-224, hsa-miR-20b, hsa-miR-1321, hsa-miR-631, hsa-miR-106a, hsa-miR-1245, hsa-miR-877*, hsa-miR-30b*, hsa-miR-483-5p, hsa-miR-520h, hsa-miR-339-5p, hsa-miR-17, hsa-miR-1279, hsa-miR-7, hsa-miR-454, hsa-miR-302b, hsa-miR-1283, hsa-miR-888*, hsa-miR-577, hsa-miR-206, hsa-miR-1298, hsa-miR-20a, hsa-miR-423-5p, hsa-miR-455-3p, hsa-miR-509-5p, hsa-miR-340, hsa-miR-193b*, hsa-miR-106b, hsa-miR-1197, hsa-miR-372, hsa-miR-1183, hsa-miR-1275, hsa-miR-1205, hsa-miR-613.

Splicing factor proline/glutamine-rich (polypyrimidine tract binding protein associated)—SFPQ—NM_{—}005066—hsa-miR-150*, hsa-miR-550*, hsa-miR-195*, hsa-miR-302c*, hsa-miR-138-1*, hsa-miR-516a-3p, hsa-miR-16-2*, hsa-miR-335*, hsa-miR-217, hsa-miR-660, hsa-miR-586, hsa-miR-582-5p, hsa-miR-1296, hsa-miR-337-3p, hsa-miR-606, hsa-miR-516b*, hsa-miR-153, hsa-miR-26b, hsa-miR-624, hsa-miR-590-3p, hsa-miR-495, hsa-miR-29c, hsa-miR-1297, hsa-miR-7-1*, hsa-miR-29b, hsa-miR-200c*, hsa-miR-509-3p, hsa-miR-1226, hsa-miR-29a, hsa-miR-20a*, hsa-miR-607, hsa-miR-200a, hsa-miR-545*, hsa-miR-141, hsa-miR-381, hsa-miR-656, hsa-miR-655, hsa-miR-7-2*, hsa-miR-300, hsa-miR-944, hsa-let-7f-2*, hsa-miR-26a.

Cbp/p300-interacting transactivator, with Glu/Asp-rich carboxy-terminal domain, 4—CITED4—NM_{—}133467—hsa-miR-1254, hsa-miR-625, hsa-miR-130b*, hsa-miR-650, hsa-miR-548g, hsa-miR-214, hsa-miR-127-5p, hsa-miR-320c, hsa-miR-486-3p, hsa-miR-658, hsa-miR-1281, hsa-miR-220c, hsa-miR-1324, hsa-miR-320d, hsa-miR-1291, hsa-miR-885-3p, hsa-miR-768-5p, hsa-miR-608, hsa-miR-548b-3p, hsa-miR-330-5p, hsa-miR-760, hsa-miR-151-5p, hsa-miR-185, hsa-miR-1256, hsa-miR-30b*, hsa-miR-320a, hsa-miR-24, hsa-miR-106a*, hsa-miR-320b, hsa-miR-485-5p, hsa-miR-140-3p, hsa-miR-1197, hsa-miR-326, hsa-miR-1275, hsa-miR-744.

CCAAT/enhancer binding protein (C/EBP), delta—CEBPD—NM_{—}005195—hsa-miR-200b, hsa-miR-509-3-5p, hsa-miR-1184, hsa-miR-604, hsa-miR-647, hsa-miR-29b-1*, hsa-miR-885-5p, hsa-miR-1207-5p, hsa-miR-524-5p, hsa-miR-202, hsa-miR-200c, hsa-miR-429, hsa-miR-105, hsa-miR-607, hsa-miR-942, hsa-miR-572, hsa-miR-941, hsa-miR-760, hsa-miR-202*, hsa-miR-627, hsa-miR-509-5p, hsa-miR-520d-5p.

Eukaryotic translation initiation factor 4E family member 2—EIF4E2—NM_{—}004846—hsa-miR-141*, hsa-miR-1236, hsa-miR-520a-5p, hsa-miR-525-5p, hsa-miR-29c, hsa-miR-589*, hsa-miR-26b*, hsa-miR-29b, hsa-miR-29a, hsa-miR-1245, hsa-miR-767-5p.

Heparan sulfate 2-O-sulfotransferase 1—HS2ST1—NM_{—}012262—hsa-miR-576-3p, hsa-miR-140-5p, hsa-miR-502-3p, hsa-miR-518d-5p, hsa-miR-200b, hsa-miR-520b, hsa-miR-105*, hsa-miR-34c-5p, hsa-miR-518a-3p, hsa-miR-380, hsa-miR-16, hsa-miR-448, hsa-miR-519a*, hsa-miR-515-5p, hsa-miR-380*, hsa-miR-141*, hsa-miR-542-3p, hsa-miR-522*, hsa-miR-922, hsa-miR-625, hsa-miR-216a, hsa-miR-219-1-3p, hsa-let-7d, hsa-miR-513a-5p, hsa-miR-650, hsa-miR-1276, hsa-miR-133a, hsa-miR-520g, hsa-miR-222*, hsa-miR-374b, hsa-miR-181a, hsa-miR-551 b*, hsa-miR-513b, hsa-miR-182*, hsa-miR-195*, hsa-miR-487a, hsa-miR-548g, hsa-miR-204, hsa-miR-563, hsa-miR-214, hsa-miR-377, hsa-miR-30c-1*, hsa-miR-1304, hsa-miR-548a-5p, hsa-miR-559, hsa-miR-1267, hsa-miR-138-1*, hsa-miR-127-5p, hsa-miR-548b-5p, hsa-miR-516a-3p, hsa-miR-452, hsa-miR-16-2*, hsa-miR-575, hsa-miR-320c, hsa-miR-520a-3p, hsa-miR-15b*, hsa-miR-31*, hsa-miR-513a-3p, hsa-miR-544, hsa-miR-26a-1*, hsa-miR-142-5p, hsa-miR-449a, hsa-miR-548m, hsa-miR-330-3p, hsa-miR-28-5p, hsa-miR-190, hsa-miR-302a*, hsa-miR-561, hsa-miR-19b, hsa-miR-541*, hsa-miR-548n, hsa-miR-1178, hsa-miR-146b-3p, hsa-miR-302e, hsa-let-7c*, hsa-miR-548c-5p, hsa-miR-603, hsa-miR-1825, hsa-miR-298, hsa-miR-508-5p, hsa-miR-130b, hsa-miR-619, hsa-let-7e, hsa-miR-1207-5p, hsa-miR-875-3p, hsa-miR-216b, hsa-miR-630, hsa-miR-129*, hsa-miR-148a, hsa-miR-600, hsa-miR-576-5p, hsa-miR-548p, hsa-miR-586, hsa-miR-876-5p, hsa-miR-338-5p, hsa-miR-524-5p, hsa-miR-30b, hsa-miR-543, hsa-miR-186, hsa-miR-23a, hsa-miR-365, hsa-miR-301b, hsa-miR-30c-2*, hsa-miR-449b, hsa-miR-1236, hsa-miR-1305, hsa-miR-33a*, hsa-miR-302c, hsa-miR-548i, hsa-miR-520a-5p, hsa-miR-516b*, hsa-miR-646, hsa-miR-708, hsa-miR-146a*, hsa-miR-153, hsa-miR-525-5p, hsa-miR-494, hsa-miR-582-3p, hsa-miR-30a*, hsa-miR-30a, hsa-miR-489, hsa-miR-373, hsa-miR-200c, hsa-miR-130a, hsa-miR-145, hsa-miR-148b, hsa-miR-548f, hsa-miR-569, hsa-miR-518b, hsa-miR-664, hsa-let-7f, hsa-miR-573, hsa-miR-302d, hsa-miR-1202, hsa-miR-15b, hsa-miR-320d, hsa-miR-590-3p, hsa-miR-29c, hsa-miR-513c, hsa-miR-7-1*, hsa-miR-1208, hsa-miR-767-3p, hsa-miR-589*, hsa-miR-26b*, hsa-miR-1253, hsa-miR-190b, hsa-miR-34a, hsa-miR-936, hsa-miR-921, hsa-miR-29b, hsa-miR-579, hsa-miR-26a-2*, hsa-miR-361-5p, hsa-miR-520f, hsa-miR-520c-3p, hsa-miR-518a-5p, hsa-miR-770-5p, hsa-miR-633, hsa-miR-144, hsa-miR-653, hsa-miR-302a, hsa-miR-888, hsa-miR-30d*, hsa-miR-568, hsa-miR-30e*, hsa-miR-596, hsa-miR-429, hsa-miR-29a, hsa-miR-570, hsa-miR-105, hsa-let-7g, hsa-miR-124, hsa-miR-940, hsa-miR-548d-5p, hsa-miR-34b, hsa-miR-1294, hsa-miR-518f*, hsa-miR-520d-3p, hsa-miR-195, hsa-miR-331-5p, hsa-miR-519d, hsa-miR-198, hsa-miR-452*, hsa-miR-181d, hsa-miR-514, hsa-miR-548j, hsa-miR-607, hsa-miR-520e, hsa-miR-520c-5p, hsa-miR-1258, hsa-miR-200a, hsa-miR-30d, hsa-miR-629*, hsa-miR-93, hsa-miR-383, hsa-miR-29b-2*, hsa-let-7i, hsa-miR-942, hsa-miR-152, hsa-miR-23b, hsa-miR-545*, hsa-miR-539, hsa-miR-548a-3p, hsa-miR-634, hsa-miR-133b, hsa-let-7b, hsa-miR-149*, hsa-miR-328, hsa-miR-301a, hsa-miR-141, hsa-let-7a, hsa-miR-526b*, hsa-miR-518c, hsa-miR-98, hsa-miR-203, hsa-miR-548h, hsa-miR-492, hsa-miR-381, hsa-miR-1185, hsa-miR-211, hsa-miR-519e*, hsa-miR-518e*, hsa-miR-593*, hsa-miR-30c, hsa-miR-181c, hsa-miR-526a, hsa-miR-583, hsa-miR-527, hsa-miR-185, hsa-miR-20b, hsa-miR-523*, hsa-miR-1224-3p, hsa-miR-106a, hsa-miR-1245, hsa-miR-138-2*, hsa-miR-130a*, hsa-miR-520h, hsa-miR-320a, hsa-miR-522, hsa-let-7c, hsa-miR-1228, hsa-miR-129-3p, hsa-miR-1827, hsa-miR-548c-3p, hsa-miR-17, hsa-miR-518f, hsa-miR-196a*, hsa-miR-500*, hsa-miR-1279, hsa-miR-106a*, hsa-miR-656, hsa-miR-320b, hsa-miR-454, hsa-miR-345, hsa-miR-548k, hsa-miR-5481, hsa-miR-424, hsa-miR-506, hsa-miR-302b, hsa-miR-181b, hsa-miR-1283, hsa-miR-187*, hsa-miR-548e, hsa-miR-1251, hsa-miR-518d-3p, hsa-miR-137, hsa-miR-1278, hsa-miR-888*, hsa-miR-892a, hsa-miR-488*, hsa-miR-577, hsa-miR-7-2*, hsa-miR-196a, hsa-miR-19a, hsa-miR-188-5p, hsa-miR-300, hsa-miR-15a, hsa-miR-485-3p, hsa-miR-374a, hsa-miR-556-3p, hsa-miR-20a, hsa-miR-183, hsa-miR-455-3p, hsa-miR-196b, hsa-miR-126*, hsa-miR-519b-5p, hsa-miR-767-5p, hsa-miR-944, hsa-miR-501 -3p, hsa-miR-340, hsa-miR-140-3p, hsa-miR-106b, hsa-miR-497, hsa-miR-369-3p, hsa-miR-296-3p, hsa-miR-1197, hsa-miR-519c-5p, hsa-miR-16-1*, hsa-miR-372, hsa-miR-497*, hsa-miR-223*, hsa-miR-433, hsa-miR-154, hsa-miR-499-3p, hsa-miR-1275, hsa-miR-520d-5p, hsa-miR-1205, hsa-miR-891b, hsa-miR-154*, hsa-miR-30e.

Alkylglycerone phosphate synthase—AGPS—NM_{—}003659—hsa-miR-124*, hsa-miR-557, hsa-miR-518d-5p, hsa-miR-637, hsa-miR-510, hsa-miR-200b, hsa-miR-520b, hsa-miR-1266, hsa-miR-32, hsa-miR-448, hsa-miR-519a*, hsa-miR-500, hsa-miR-20b*, hsa-miR-141*, hsa-miR-522*, hsa-miR-625, hsa-miR-562, hsa-miR-513a-5p, hsa-miR-125b-2*, hsa-miR-616, hsa-miR-222*, hsa-miR-551b*, hsa-miR-513b, hsa-miR-182*, hsa-miR-195*, hsa-miR-221, hsa-miR-483-3p, hsa-miR-204, hsa-miR-1289, hsa-miR-1179, hsa-miR-532-3p, hsa-miR-377, hsa-miR-1304, hsa-miR-890, hsa-miR-548a-5p, hsa-miR-1826, hsa-miR-647, hsa-miR-559, hsa-miR-138-1*, hsa-miR-548b-5p, hsa-miR-516a-3p, hsa-miR-452, hsa-miR-16-2*, hsa-miR-320c, hsa-miR-520a-3p, hsa-miR-511, hsa-miR-15b*, hsa-miR-31*, hsa-miR-513a-3p, hsa-miR-544, hsa-miR-26a-1*, hsa-miR-142-5p, hsa-miR-220a, hsa-miR-24-1*, hsa-miR-486-3p, hsa-miR-335*, hsa-miR-507, hsa-miR-548m, hsa-miR-183*, hsa-miR-190, hsa-miR-302a*, hsa-miR-1237, hsa-miR-943, hsa-miR-541*, hsa-miR-548n, hsa-miR-1178, hsa-miR-217, hsa-miR-302e, hsa-miR-93*, hsa-miR-548c-5p, hsa-miR-603, hsa-miR-342-3p, hsa-miR-92b, hsa-miR-587, hsa-miR-199a-5p, hsa-miR-216b, hsa-miR-1252, hsa-miR-1182, hsa-miR-129*, hsa-miR-1246, hsa-miR-600, hsa-miR-576-5p, hsa-miR-548p, hsa-miR-586, hsa-miR-876-5p, hsa-miR-367, hsa-miR-582-5p, hsa-miR-338-5p, hsa-miR-30b, hsa-miR-543, hsa-miR-25, hsa-miR-186, hsa-miR-302c, hsa-miR-548i, hsa-miR-218-2*, hsa-miR-606, hsa-miR-410, hsa-miR-421, hsa-miR-24-2*, hsa-miR-641, hsa-miR-516b*, hsa-miR-646, hsa-miR-329, hsa-miR-494, hsa-miR-30a, hsa-miR-29a*, hsa-miR-373, hsa-miR-200c, hsa-miR-581, hsa-miR-1207-3p, hsa-miR-616*, hsa-miR-373*, hsa-miR-26b, hsa-miR-664, hsa-miR-302d, hsa-miR-624, hsa-miR-1324, hsa-miR-320d, hsa-miR-590-3p, hsa-miR-502-5p, hsa-miR-495, hsa-miR-29c, hsa-miR-1297, hsa-miR-7-1*, hsa-miR-1208, hsa-miR-589*, hsa-miR-26b*, hsa-miR-1253, hsa-miR-190b, hsa-miR-921, hsa-miR-643, hsa-miR-29b, hsa-miR-135a, hsa-miR-579, hsa-miR-26a-2*, hsa-miR-361-5p, hsa-miR-520f, hsa-miR-605, hsa-miR-768-5p, hsa-miR-1259, hsa-miR-374b*, hsa-miR-186*, hsa-miR-1233, hsa-miR-520c-3p, hsa-miR-518a-5p, hsa-miR-27a*, hsa-miR-1226, hsa-miR-185*, hsa-miR-633, hsa-miR-144, hsa-miR-384, hsa-miR-302a, hsa-miR-429, hsa-miR-29a, hsa-miR-570, hsa-miR-105, hsa-miR-124, hsa-miR-548d-5p, hsa-miR-222, hsa-miR-432, hsa-miR-593, hsa-miR-518f*, hsa-miR-520d-3p, hsa-miR-18b*, hsa-miR-331-5p, hsa-miR-610, hsa-miR-92a-1*, hsa-miR-198, hsa-miR-20a*, hsa-miR-935, hsa-miR-135b, hsa-miR-9*, hsa-miR-548j, hsa-miR-323-3p, hsa-miR-607, hsa-miR-520e, hsa-miR-520c-5p, hsa-miR-200a, hsa-miR-578, hsa-miR-30d, hsa-miR-29b-2*, hsa-miR-1206, hsa-miR-518c*, hsa-miR-491-3p, hsa-miR-942, hsa-miR-101, hsa-miR-362-3p, hsa-miR-634, hsa-miR-526b, hsa-miR-139-5p, hsa-miR-141, hsa-miR-889, hsa-miR-22*, hsa-miR-203, hsa-miR-548h, hsa-miR-92a, hsa-miR-381, hsa-miR-708*, hsa-miR-211, hsa-miR-518e*, hsa-miR-593*, hsa-miR-150, hsa-miR-30c, hsa-miR-526a, hsa-miR-181a-2*, hsa-miR-583, hsa-miR-501-5p, hsa-miR-199b-5p, hsa-miR-221*, hsa-miR-527, hsa-miR-378*, hsa-miR-556-5p, hsa-miR-523*, hsa-miR-199a-3p, hsa-miR-320a, hsa-miR-129-3p, hsa-miR-1827, hsa-miR-24, hsa-miR-1299, hsa-miR-548c-3p, hsa-miR-196a*, hsa-miR-1279, hsa-miR-106a*, hsa-miR-7, hsa-miR-382, hsa-miR-656, hsa-miR-320b, hsa-miR-1264, hsa-miR-5481, hsa-miR-506, hsa-miR-302b, hsa-miR-1283, hsa-miR-187*, hsa-miR-655, hsa-miR-199b-3p, hsa-miR-1278, hsa-miR-888*, hsa-miR-924, hsa-miR-892a, hsa-miR-7-2*, hsa-miR-377*, hsa-miR-300, hsa-miR-1244, hsa-miR-627, hsa-miR-876-3p, hsa-miR-126*, hsa-miR-363, hsa-miR-519b-5p, hsa-miR-1272, hsa-miR-767-5p, hsa-miR-944, hsa-miR-340, hsa-miR-1265, hsa-miR-499-5p, hsa-miR-219-2-3p, hsa-miR-140-3p, hsa-let-7f-2*, hsa-miR-519c-5p, hsa-miR-649, hsa-miR-372, hsa-miR-367*, hsa-miR-497*, hsa-miR-154, hsa-miR-26a, hsa-miR-1275, hsa-miR-891b, hsa-miR-30e.

Phosphoglycerate kinase 1—PGK1—NM_{—}000291—hsa-miR-219-1-3p, hsa-miR-452, hsa-miR-26a-1*, hsa-miR-142-5p, hsa-miR-1178, hsa-miR-217, hsa-miR-338-5p, hsa-miR-143*, hsa-miR-186, hsa-miR-202, hsa-miR-494, hsa-miR-324-3p, hsa-miR-548o, hsa-miR-96*, hsa-miR-767-3p, hsa-miR-26a-2*, hsa-miR-146a, hsa-miR-873, hsa-miR-185*, hsa-miR-1323, hsa-miR-654-3p, hsa-miR-517*, hsa-miR-940, hsa-miR-588, hsa-miR-1270, hsa-miR-769-3p, hsa-miR-768-3p, hsa-miR-450b-3p, hsa-miR-607, hsa-miR-200a, hsa-miR-23a*, hsa-miR-23b*, hsa-miR-1229, hsa-miR-146b-5p, hsa-miR-620, hsa-miR-330-5p, hsa-miR-141, hsa-miR-221*, hsa-miR-556-5p, hsa-miR-30b*, hsa-miR-758, hsa-miR-548c-3p, hsa-miR-5481, hsa-miR-1283, hsa-miR-632, hsa-miR-346, hsa-miR-326.

5-aminoimidazole-4-carboxamide ribonucleotide formyltrans-ferase/IMP cyclohydrolase—ATIC—NM_{—}004044—hsa-miR-101*, hsa-miR-452, hsa-miR-561, hsa-miR-943, hsa-miR-134, hsa-miR-147.

Ethanolamine kinase 1—ETNK1—Variant 1—NM_{—}018638—hsa-miR-642, hsa-miR-380, hsa-miR-16, hsa-miR-448, hsa-miR-20b*, hsa-miR-515-5p, hsa-miR-27b*, hsa-miR-542-3p, hsa-miR-625, hsa-miR-493*, hsa-miR-562, hsa-let-7d, hsa-miR-513a-5p, hsa-miR-125b-2*, hsa-miR-150*, hsa-miR-222*, hsa-miR-374b, hsa-miR-181a, hsa-miR-551b*, hsa-miR-519b-3p, hsa-miR-513b, hsa-miR-182*, hsa-miR-195*, hsa-miR-302c*, hsa-miR-487a, hsa-miR-337-5p, hsa-miR-548g, hsa-miR-193a-5p, hsa-miR-532-3p, hsa-miR-377, hsa-miR-218-1*, hsa-miR-548a-5p, hsa-miR-376c, hsa-miR-559, hsa-miR-138-1*, hsa-miR-127-5p, hsa-miR-548b-5p, hsa-miR-16-2*, hsa-miR-496, hsa-miR-511, hsa-miR-15b*, hsa-miR-31*, hsa-miR-513a-3p, hsa-miR-148a*, hsa-miR-544, hsa-miR-142-5p, hsa-miR-379*, hsa-miR-335*, hsa-miR-330-3p, hsa-miR-28-5p, hsa-miR-561, hsa-miR-519c-3p, hsa-miR-19b, hsa-miR-217, hsa-miR-548c-5p, hsa-miR-519a, hsa-miR-130b, hsa-miR-587, hsa-let-7e, hsa-miR-1303, hsa-miR-216b, hsa-miR-103, hsa-miR-580, hsa-miR-600, hsa-miR-576-5p, hsa-miR-548p, hsa-miR-876-5p, hsa-miR-582-5p, hsa-miR-338-5p, hsa-miR-524-5p, hsa-miR-30b, hsa-miR-23 a, hsa-miR-365, hsa-miR-301b, hsa-miR-1305, hsa-miR-33a*, hsa-miR-548i, hsa-miR-218-2*, hsa-miR-202, hsa-miR-606, hsa-miR-410, hsa-miR-421, hsa-miR-641, hsa-miR-212, hsa-miR-548d-3p, hsa-let-7b*, hsa-miR-708, hsa-miR-146a*, hsa-miR-153, hsa-miR-494, hsa-miR-30a*, hsa-miR-371-5p, hsa-miR-30a, hsa-miR-489, hsa-miR-130a, hsa-miR-145, hsa-miR-548f, hsa-miR-26b, hsa-miR-664, hsa-let-7f, hsa-miR-32*, hsa-miR-15b, hsa-miR-1324, hsa-miR-188-3p, hsa-miR-590-3p, hsa-miR-132, hsa-miR-1297, hsa-miR-7-1*, hsa-miR-1208, hsa-miR-767-3p, hsa-miR-589*, hsa-miR-579, hsa-miR-411*, hsa-miR-1259, hsa-miR-376a*, hsa-miR-518a-5p, hsa-miR-144, hsa-miR-384, hsa-miR-888, hsa-miR-30d*, hsa-miR-802, hsa-miR-568, hsa-miR-30e*, hsa-miR-592, hsa-miR-596, hsa-miR-570, hsa-let-7g, hsa-miR-517*, hsa-miR-548d-5p, hsa-miR-34b, hsa-miR-325, hsa-miR-768-3p, hsa-miR-125a-3p, hsa-miR-512-3p, hsa-miR-18b*, hsa-miR-195, hsa-miR-331-5p, hsa-miR-610, hsa-miR-545, hsa-miR-181d, hsa-miR-935, hsa-miR-9*, hsa-miR-548j, hsa-miR-607, hsa-miR-1258, hsa-miR-200a, hsa-miR-30d, hsa-miR-200b*, hsa-miR-518c*, hsa-let-7i, hsa-miR-942, hsa-miR-23b, hsa-miR-101, hsa-miR-545*, hsa-miR-539, hsa-miR-1257, hsa-miR-548a-3p, hsa-miR-526b, hsa-let-7b, hsa-miR-301a, hsa-miR-141, hsa-let-7a, hsa-miR-98, hsa-miR-203, hsa-miR-548h, hsa-miR-381, hsa-miR-708*, hsa-miR-597, hsa-miR-1185, hsa-miR-519e*, hsa-let-7f-1*, hsa-miR-30c, hsa-miR-181c, hsa-miR-205, hsa-miR-1284, hsa-miR-659, hsa-miR-527, hsa-miR-185, hsa-miR-556-5p, hsa-miR-100*, hsa-miR-199a-3p, hsa-miR-138-2*, hsa-miR-130a*, hsa-miR-522, hsa-miR-555, hsa-let-7c, hsa-miR-24, hsa-miR-1299, hsa-miR-548c-3p, hsa-miR-196a*, hsa-miR-1279, hsa-miR-656, hsa-miR-454, hsa-miR-1264, hsa-miR-5481, hsa-miR-424, hsa-miR-181b, hsa-miR-1283, hsa-miR-548e, hsa-miR-655, hsa-miR-485-5p, hsa-miR-199b-3p, hsa-miR-1278, hsa-miR-888*, hsa-miR-892a, hsa-miR-577, hsa-miR-7-2*, hsa-miR-107, hsa-miR-19a, hsa-miR-188-5p, hsa-miR-300, hsa-miR-15a, hsa-miR-485-3p, hsa-miR-374a, hsa-miR-556-3p, hsa-miR-425, hsa-miR-876-3p, hsa-miR-126*, hsa-let-7a*, hsa-miR-767-5p, hsa-miR-944, hsa-miR-340, hsa-miR-140-3p, hsa-miR-497, hsa-let-7f-2*, hsa-miR-16-1*, hsa-miR-367*, hsa-miR-1183, hsa-miR-10a*, hsa-miR-223*, hsa-miR-433, hsa-miR-505, hsa-miR-154, hsa-miR-499-3p, hsa-miR-26a, hsa-miR-1275, hsa-miR-520d-5p, hsa-miR-200a*, hsa-miR-1302, hsa-miR-154*, hsa-miR-584, hsa-miR-30e.

ETNK1—Variant 2—NM_{—}001039481—hsa-miR-31, hsa-miR-146a*, hsa-miR-371-5p, hsa-miR-590-3p, hsa-miR-888, hsa-miR-568, hsa-miR-570.

Asparagine-linked glycosylation 2 homolog—ALG2—NM_{—}033087—hsa-miR-200b, hsa-miR-625, hsa-miR-648, hsa-miR-219-1-3p, hsa-miR-192, hsa-miR-150*, hsa-miR-181a, hsa-miR-195*, hsa-miR-548g, hsa-miR-96, hsa-miR-138-1*, hsa-miR-452, hsa-miR-16-2*, hsa-miR-15b*, hsa-miR-544, hsa-miR-28-5p, hsa-miR-302a*, hsa-miR-19b, hsa-miR-93*, hsa-miR-603, hsa-miR-875-3p, hsa-miR-103, hsa-miR-660, hsa-miR-576-5p, hsa-miR-338-5p, hsa-miR-30b, hsa-miR-23a, hsa-miR-337-3p, hsa-miR-202, hsa-miR-606, hsa-miR-641, hsa-miR-708, hsa-miR-30a, hsa-miR-29a*, hsa-miR-548o, hsa-miR-200c, hsa-miR-145, hsa-miR-548f, hsa-miR-32*, hsa-miR-628-3p, hsa-miR-599, hsa-miR-1271, hsa-miR-513c, hsa-miR-215, hsa-miR-376a*, hsa-miR-144, hsa-miR-1323, hsa-miR-653, hsa-miR-429, hsa-miR-124, hsa-miR-1270, hsa-miR-20a*, hsa-miR-181d, hsa-miR-200a, hsa-miR-30d, hsa-miR-29b-2*, hsa-miR-23b, hsa-miR-548a-3p, hsa-miR-567, hsa-miR-620, hsa-miR-141, hsa-miR-597, hsa-miR-30c, hsa-miR-181c, hsa-miR-1321, hsa-miR-30b*, hsa-miR-130a*, hsa-miR-1299, hsa-miR-656, hsa-miR-506, hsa-miR-181b, hsa-miR-548e, hsa-miR-577, hsa-miR-107, hsa-miR-19a, hsa-miR-425, hsa-miR-1225-3p, hsa-miR-30e.

Ubiquitin-conjugating enzyme E2F—UBE2F—NM_{—}080678—hsa-miR-513a-5p, hsa-miR-548g, hsa-miR-549, hsa-miR-548a-5p, hsa-miR-559, hsa-miR-27a, hsa-miR-548b-5p, hsa-miR-548n, hsa-miR-548c-5p, hsa-miR-1303, hsa-miR-1207-5p, hsa-miR-580, hsa-miR-586, hsa-miR-30b, hsa-miR-543, hsa-miR-548i, hsa-let-7b*, hsa-miR-30a, hsa-miR-653, hsa-miR-1224-5p, hsa-miR-940, hsa-miR-548d-5p, hsa-miR-27b, hsa-miR-548j, hsa-miR-30d, hsa-miR-192*, hsa-miR-101, hsa-miR-1257, hsa-miR-139-5p, hsa-miR-548h, hsa-let-7f-1*, hsa-miR-30c, hsa-miR-1284, hsa-miR-30b*, hsa-miR-196a*, hsa-miR-632, hsa-miR-455-3p, hsa-let-7a*, hsa-miR-219-2-3p, hsa- let-7f-2*, hsa-miR-30e.

Membrane-associated ring finger (C3HC4) 8—MARCH8—Variant 6, Variant 7 and Variant 8—NM_{—}001002265, NM_{—}001002266 and NM_{—}145021—hsa-miR-520b, hsa-miR-490-5p, hsa-miR-22, hsa-miR-650, hsa-miR-1290, hsa-miR-193a-5p, hsa-miR-335, hsa-miR-520a-3p, hsa-miR-661, hsa-miR-15b*, hsa-miR-548m, hsa-miR-129-5p, hsa-miR-302e, hsa-miR-623, hsa-miR-103, hsa-miR-1246, hsa-miR-524-5p, hsa-miR-30b, hsa-miR-186, hsa-miR-1236, hsa-miR-302c, hsa-let-7b*, hsa-miR-153, hsa-miR-30a, hsa-miR-373, hsa-miR-1286, hsa-miR-302d, hsa-miR-765, hsa-miR-188-3p, hsa-miR-29c, hsa-miR-591, hsa-miR-767-3p, hsa-miR-29b, hsa-miR-873, hsa-miR-520c-3p, hsa-miR-302a, hsa-miR-29a, hsa-miR-940, hsa-miR-1285, hsa-miR-512-3p, hsa-miR-520d-3p, hsa-miR-519d, hsa-miR-612, hsa-miR-520e, hsa-miR-30d, hsa-miR-629, hsa-miR-93, hsa-miR-545*, hsa-miR-526b*, hsa-let-7f-1*, hsa-miR-30c, hsa-miR-20b, hsa-miR-106a, hsa-miR-877*, hsa-miR-149, hsa-miR-1827, hsa-miR-17, hsa-miR-302b, hsa-miR-107, hsa-miR-20a, hsa-miR-627, hsa-let-7a*, hsa-miR-767-5p, hsa-miR-140-3p, hsa-miR-106b, hsa-miR-372, hsa-miR-433, hsa-miR-520d-5p, hsa-miR-558, hsa-miR-30e.

CCR4-NOT transcription complex, subunit 4—CNOT4—Variant 1—NM_{—}013316—hsa-miR-200b, hsa-miR-216a, hsa-miR-181a, hsa-miR-668, hsa-miR-1826, hsa-miR-490-3p, hsa-miR-920, hsa-miR-320c, hsa-miR-511, hsa-miR-544, hsa-miR-143, hsa-miR-335*, hsa-miR-29b-1*, hsa-miR-1287, hsa-miR-576-5p, hsa-miR-586, hsa-miR-543, hsa-miR-186, hsa-miR-337-3p, hsa-miR-10a, hsa-miR-494, hsa-miR-200c, hsa-let-7i*, hsa-miR-320d, hsa-miR-502-5p, hsa-miR-589*, hsa-miR-1243, hsa-miR-888, hsa-miR-429, hsa-miR-570, hsa-miR-105, hsa-miR-125a-3p, hsa-miR-331-5p, hsa-miR-198, hsa-miR-20a*, hsa-miR-181d, hsa-miR-504, hsa-miR-935, hsa-miR-383, hsa-miR-1229, hsa-miR-381, hsa-miR-597, hsa-miR-1300, hsa-miR-181c, hsa-miR-583, hsa-miR-17*, hsa-miR-18a*, hsa-miR-320a, hsa-miR-10b, hsa-miR-656, hsa-miR-320b, hsa-miR-654-5p, hsa-miR-181b, hsa-miR-300, hsa-miR-425, hsa-miR-126*, hsa-miR-154, hsa-miR-541, hsa-miR-1205, hsa-miR-1302, hsa-miR-1293.

CNOT4—Variant 2—NM_{—}001008225—hsa-miR-34c-5p, hsa-miR-380, hsa-miR-448, hsa-miR-216a, hsa-miR-519b-3p, hsa-miR-549, hsa-miR-1304, hsa-miR-15b*, hsa-miR-148a*, hsa-miR-449a, hsa-miR-1237, hsa-miR-519c-3p, hsa-miR-19b, hsa-miR-885-5p, hsa-miR-519a, hsa-miR-130b, hsa-miR-651, hsa-miR-630, hsa-miR-148a, hsa-miR-548p, hsa-miR-338-5p, hsa-miR-186, hsa-miR-301b, hsa-miR-449b, hsa-miR-33a*, hsa-miR-130a, hsa-miR-145, hsa-miR-148b, hsa-miR-26b, hsa-miR-513c, hsa-miR-1297, hsa-miR-34a, hsa-miR-220b, hsa-miR-568, hsa-miR-570, hsa-miR-769-3p, hsa-miR-34b*, hsa-miR-148b*, hsa-miR-450b-3p, hsa-miR-450b-5p, hsa-miR-519d, hsa-miR-93, hsa-miR-152, hsa-miR-1229, hsa-miR-301a, hsa-miR-526b*, hsa-miR-20b, hsa-miR-106a, hsa-miR-548c-3p, hsa-miR-17, hsa-miR-656, hsa-miR-454, hsa-miR-187*, hsa-miR-19a, hsa-miR-20a, hsa-miR-425, hsa-miR-126*, hsa-miR-944, hsa-miR-106b, hsa-miR-26a.

Ring finger protein 8—RNF8—Variant 1—NM_{—}003958—hsa-miR-124*, hsa-miR-509-3-5p, hsa-miR-520b, hsa-miR-32, hsa-miR-1254, hsa-miR-16, hsa-miR-20b*, hsa-miR-515-5p, hsa-miR-922, hsa-miR-625, hsa-miR-493*, hsa-miR-138, hsa-miR-130b*, hsa-miR-216a, hsa-let-7d, hsa-miR-513a-5p, hsa-miR-766, hsa-miR-192, hsa-miR-1276, hsa-miR-1184, hsa-miR-520g, hsa-miR-150*, hsa-miR-1301, hsa-miR-181a, hsa-miR-551b*, hsa-miR-302c*, hsa-miR-483-3p, hsa-miR-96, hsa-miR-668, hsa-miR-28-3p, hsa-miR-214, hsa-miR-532-3p, hsa-miR-377, hsa-miR-548a-5p, hsa-miR-1826, hsa-miR-647, hsa-miR-559, hsa-miR-27a, hsa-miR-1248, hsa-miR-127-5p, hsa-miR-548b-5p, hsa-miR-516a-3p, hsa-miR-496, hsa-miR-320c, hsa-miR-520a-3p, hsa-miR-511, hsa-miR-31*, hsa-miR-143, hsa-miR-24-1*, hsa-miR-548m, hsa-miR-183*, hsa-miR-330-3p, hsa-miR-1226*, hsa-miR-19b, hsa-miR-129-5p, hsa-miR-548n, hsa-miR-302e, hsa-miR-548c-5p, hsa-miR-938, hsa-miR-134, hsa-miR-342-3p, hsa-miR-1268, hsa-miR-298, hsa-miR-508-5p, hsa-miR-92b, hsa-miR-1231, hsa-let-7e, hsa-miR-370, hsa-miR-1303, hsa-miR-875-3p, hsa-miR-657, hsa-miR-103, hsa-miR-1252, hsa-miR-129*, hsa-miR-576-5p, hsa-miR-367, hsa-miR-582-5p, hsa-miR-338-5p, hsa-miR-524-5p, hsa-miR-30b, hsa-miR-543, hsa-miR-25, hsa-miR-365, hsa-miR-10a, hsa-miR-302c, hsa-miR-491-5p, hsa-miR-548i, hsa-miR-202, hsa-miR-24-2*, hsa-miR-516b*, hsa-miR-646, hsa-let-7b*, hsa-miR-146a*, hsa-miR-494, hsa-miR-411, hsa-miR-30a, hsa-miR-622, hsa-miR-1, hsa-miR-373, hsa-miR-145, hsa-miR-616*, hsa-miR-373*, hsa-miR-484, hsa-let-7f, hsa-miR-32*, hsa-miR-302d, hsa-miR-1202, hsa-miR-624, hsa-miR-15b, hsa-miR-320d, hsa-miR-188-3p, hsa-miR-495, hsa-miR-1271, hsa-miR-7-1 *, hsa-miR-215, hsa-miR-635, hsa-miR-589*, hsa-miR-921, hsa-miR-643, hsa-miR-135a, hsa-miR-361-5p, hsa-miR-520f, hsa-miR-146a, hsa-miR-605, hsa-miR-1243, hsa-miR-1233, hsa-miR-520c-3p, hsa-miR-518a-5p, hsa-miR-18b, hsa-miR-185*, hsa-miR-144, hsa-miR-384, hsa-miR-302a, hsa-miR-888, hsa-miR-802, hsa-miR-654-3p, hsa-miR-122*, hsa-miR-503, hsa-miR-488, hsa-miR-105, hsa-let-7g, hsa-miR-940, hsa-miR-548d-5p, hsa-miR-34b, hsa-miR-1294, hsa-miR-588, hsa-miR-593, hsa-miR-768-3p, hsa-miR-34b*, hsa-miR-520d-3p, hsa-miR-195, hsa-miR-519d, hsa-miR-20a*, hsa-miR-452*, hsa-miR-181d, hsa-miR-935, hsa-miR-135b, hsa-miR-27b, hsa-miR-548j, hsa-miR-607, hsa-miR-520e, hsa-miR-30d, hsa-miR-629*, hsa-miR-93, hsa-miR-1206, hsa-miR-518c*, hsa-miR-548b-3p, hsa-miR-1260, hsa-let-7i, hsa-miR-942, hsa-miR-609, hsa-miR-1255a, hsa-miR-146b-5p, hsa-let-7b, hsa-let-7a, hsa-miR-889, hsa-miR-526b*, hsa-miR-98, hsa-miR-548h, hsa-miR-92a, hsa-miR-625*, hsa-miR-381, hsa-miR-744*, hsa-miR-519e*, hsa-let-7f-1*, hsa-miR-432*, hsa-miR-30c, hsa-miR-181c, hsa-miR-760, hsa-miR-18a, hsa-miR-221*, hsa-miR-527, hsa-miR-185, hsa-miR-20b, hsa-miR-556-5p, hsa-miR-1224-3p, hsa-miR-106a, hsa-miR-30b*, hsa-miR-758, hsa-miR-520h, hsa-miR-320a, hsa-miR-10b, hsa-let-7c, hsa-miR-339-5p, hsa-miR-1228, hsa-miR-129-3p, hsa-miR-1827, hsa-miR-24, hsa-miR-1299, hsa-miR-548c-3p, hsa-miR-17, hsa-miR-196a*, hsa-miR-1279, hsa-miR-106a*, hsa-miR-382, hsa-miR-182, hsa-miR-320b, hsa-miR-297, hsa-miR-424, hsa-miR-654-5p, hsa-miR-302b, hsa-miR-181b, hsa-miR-655, hsa-miR-485-5p, hsa-miR-137, hsa-miR-888*, hsa-miR-488*, hsa-miR-577, hsa-miR-206, hsa-miR-7-2*, hsa-miR-377*, hsa-miR-107, hsa-miR-19a, hsa-miR-300, hsa-miR-15a, hsa-miR-20a, hsa-miR-627, hsa-miR-409-3p, hsa-miR-423-5p, hsa-miR-363, hsa-let-7a*, hsa-miR-338-3p, hsa-miR-509-5p, hsa-miR-106b, hsa-miR-497, hsa-miR-1197, hsa-miR-16-1*, hsa-miR-372, hsa-miR-367*, hsa-miR-223*, hsa-miR-1255b, hsa-miR-541, hsa-miR-520d-5p, hsa-miR-1302, hsa-miR-558, hsa-miR-613, hsa-miR-95, hsa-miR-30e.

RNF8—Variant 2—NM_{—}183078—hsa-miR-124*, hsa-miR-509-3-5p, hsa-miR-520b, hsa-miR-32, hsa-miR-1254, hsa-miR-16, hsa-miR-20b*, hsa-miR-515-5p, hsa-miR-922, hsa-miR-625, hsa-miR-493*, hsa-miR-138, hsa-miR-130b*, hsa-miR-216a, hsa-let-7d, hsa-miR-513a-5p, hsa-miR-766, hsa-miR-192, hsa-miR-1276, hsa-miR-1184, hsa-miR-520g, hsa-miR-150*, hsa-miR-1301, hsa-miR-181a, hsa-miR-551b*, hsa-miR-302c*, hsa-miR-483-3p, hsa-miR-96, hsa-miR-668, hsa-miR-28-3p, hsa-miR-214, hsa-miR-532-3p, hsa-miR-377, hsa-miR-548a-5p, hsa-miR-1826, hsa-miR-647, hsa-miR-559, hsa-miR-27a, hsa-miR-1248, hsa-miR-127-5p, hsa-miR-548b-5p, hsa-miR-516a-3p, hsa-miR-496, hsa-miR-320c, hsa-miR-520a-3p, hsa-miR-511, hsa-miR-31*, hsa-miR-143, hsa-miR-24-1*, hsa-miR-548m, hsa-miR-183*, hsa-miR-330-3p, hsa-miR-1226*, hsa-miR-19b, hsa-miR-129-5p, hsa-miR-548n, hsa-miR-302e, hsa-miR-548c-5p, hsa-miR-134, hsa-miR-342-3p, hsa-miR-1268, hsa-miR-298, hsa-miR-508-5p, hsa-miR-92b, hsa-miR-1231, hsa-let-7e, hsa-miR-370, hsa-miR-1303, hsa-miR-875-3p, hsa-miR-657, hsa-miR-103, hsa-miR-1252, hsa-miR-129*, hsa-miR-576-5p, hsa-miR-367, hsa-miR-582-5p, hsa-miR-338-5p, hsa-miR-524-5p, hsa-miR-30b, hsa-miR-543, hsa-miR-25, hsa-miR-10a, hsa-miR-302c, hsa-miR-491-5p, hsa-miR-548i, hsa-miR-202, hsa-miR-24-2*, hsa-miR-516b*, hsa-miR-646, hsa-let-7b*, hsa-miR-146a*, hsa-miR-494, hsa-miR-411, hsa-miR-30a, hsa-miR-622, hsa-miR-1, hsa-miR-373, hsa-miR-145, hsa-miR-616*, hsa-miR-373*, hsa-miR-484, hsa-let-7f, hsa-miR-32*, hsa-miR-302d, hsa-miR-1202, hsa-miR-624, hsa-miR-15b, hsa-miR-320d, hsa-miR-188-3p, hsa-miR-495, hsa-miR-1271, hsa-miR-7-1*, hsa-miR-215, hsa-miR-635, hsa-miR-589*, hsa-miR-921, hsa-miR-643, hsa-miR-135a, hsa-miR-361-5p, hsa-miR-520f, hsa-miR-146a, hsa-miR-605, hsa-miR-1243, hsa-miR-1233, hsa-miR-520c-3p, hsa-miR-518a-5p, hsa-miR-18b, hsa-miR-185*, hsa-miR-144, hsa-miR-384, hsa-miR-302a, hsa-miR-888, hsa-miR-802, hsa-miR-654-3p, hsa-miR-122*, hsa-miR-488, hsa-miR-105, hsa-let-7g, hsa-miR-940, hsa-miR-548d-5p, hsa-miR-34b, hsa-miR-1294, hsa-miR-588, hsa-miR-593, hsa-miR-768-3p, hsa-miR-34b*, hsa-miR-520d-3p, hsa-miR-195, hsa-miR-519d, hsa-miR-20a*, hsa-miR-452*, hsa-miR-181d, hsa-miR-935, hsa-miR-135b, hsa-miR-27b, hsa-miR-548j, hsa-miR-607, hsa-miR-520e, hsa-miR-30d, hsa-miR-629*, hsa-miR-93, hsa-miR-1206, hsa-miR-518c*, hsa-miR-548b-3p, hsa-miR-1260, hsa-let-7i, hsa-miR-942, hsa-miR-609, hsa-miR-1255a, hsa-miR-146b-5p, hsa-let-7b, hsa-let-7a, hsa-miR-889, hsa-miR-526b*, hsa-miR-98, hsa-miR-548h, hsa-miR-92a, hsa-miR-625*, hsa-miR-381, hsa-miR-744*, hsa-miR-519e*, hsa-let-7f-1*, hsa-miR-432*, hsa-miR-30c, hsa-miR-181c, hsa-miR-760, hsa-miR-18a, hsa-miR-527, hsa-miR-185, hsa-miR-20b, hsa-miR-556-5p, hsa-miR-1224-3p, hsa-miR-106a, hsa-miR-30b*, hsa-miR-758, hsa-miR-520h, hsa-miR-320a, hsa-miR-10b, hsa-let-7c, hsa-miR-339-5p, hsa-miR-1228, hsa-miR-129-3p, hsa-miR-1827, hsa-miR-24, hsa-miR-1299, hsa-miR-548c-3p, hsa-miR-17, hsa-miR-196a*, hsa-miR-1279, hsa-miR-106a*, hsa-miR-382, hsa-miR-182, hsa-miR-320b, hsa-miR-297, hsa-miR-424, hsa-miR-654-5p, hsa-miR-302b, hsa-miR-181b, hsa-miR-655, hsa-miR-485-5p, hsa-miR-137, hsa-miR-888*, hsa-miR-488*, hsa-miR-577, hsa-miR-206, hsa-miR-7-2*, hsa-miR-377*, hsa-miR-107, hsa-miR-19a, hsa-miR-300, hsa-miR-15a, hsa-miR-20a, hsa-miR-627, hsa-miR-409-3p, hsa-miR-423-5p, hsa-miR-363, hsa-let-7a*, hsa-miR-338-3p, hsa-miR-509-5p, hsa-miR-106b, hsa-miR-497, hsa-miR-1197, hsa-miR-16-1*, hsa-miR-372, hsa-miR-367*, hsa-miR-223*, hsa-miR-1255b, hsa-miR-541, hsa-miR-520d-5p, hsa-miR-1302, hsa-miR-558, hsa-miR-613, hsa-miR-95, hsa-miR-30e.

Proteasome (prosome, macropain) subunit, alpha type, 5—PSMA5—NM_{—}002790—hsa-let-7g*, hsa-miR-148a*, hsa-miR-28-5p, hsa-miR-875-3p, hsa-miR-708, hsa-miR-1270, hsa-miR-148b*, hsa-miR-620, hsa-miR-126*.

Alport syndrome, mental retardation, midface hypoplasia and elliptocytosis chromosomal region gene 1—AMMERCR1—Variant 1 and Variant 2—NM_{—}015365 and NM_{—}001025580—hsa-miR-517a, hsa-miR-637, hsa-miR-510, hsa-miR-645, hsa-miR-380, hsa-miR-16, hsa-miR-448, hsa-miR-515-5p, hsa-miR-141*, hsa-miR-542-3p, hsa-miR-922, hsa-miR-138, hsa-miR-513a-5p, hsa-miR-650, hsa-miR-1184, hsa-miR-9, hsa-miR-520g, hsa-miR-150*, hsa-miR-1290, hsa-miR-550*, hsa-miR-101*, hsa-miR-551b*, hsa-miR-454*, hsa-miR-182*, hsa-miR-195*, hsa-miR-302c*, hsa-miR-221, hsa-miR-548g, hsa-miR-204, hsa-miR-28-3p, hsa-miR-548a-5p, hsa-miR-559, hsa-miR-27a, hsa-miR-1267, hsa-miR-92a-2*, hsa-miR-548b-5p, hsa-miR-516a-3p, hsa-miR-16-2*, hsa-miR-513a-3p, hsa-miR-544, hsa-miR-143, hsa-miR-142-5p, hsa-miR-548m, hsa-miR-183*, hsa-miR-378, hsa-miR-330-3p, hsa-miR-28-5p, hsa-miR-190, hsa-miR-561, hsa-miR-191, hsa-miR-129-5p, hsa-miR-541*, hsa-miR-548n, hsa-miR-1178, hsa-miR-892b, hsa-miR-885-5p, hsa-miR-548c-5p, hsa-miR-134, hsa-miR-342-3p, hsa-miR-298, hsa-miR-508-5p, hsa-miR-130b, hsa-miR-1303, hsa-miR-1207-5p, hsa-miR-216b, hsa-miR-103, hsa-let-7d*, hsa-miR-1252, hsa-miR-1182, hsa-miR-1246, hsa-miR-600, hsa-miR-548p, hsa-miR-876-5p, hsa-miR-582-5p, hsa-miR-338-5p, hsa-mik-524-5-p, hsa-miR-143*, hsa-miR-23a, hsa-miR-365, hsa-miR-301b, hsa-miR-1236, hsa-miR-618, hsa-miR-1305, hsa-miR-33a*, hsa-miR-548i, hsa-miR-606, hsa-miR-641, hsa-miR-548d-3p, hsa-miR-516b*, hsa-let-7b*, hsa-miR-708, hsa-miR-622, hsa-miR-548o, hsa-miR-130a, hsa-miR-616*, hsa-miR-552, hsa-miR-373*, hsa-miR-548f, hsa-miR-26b, hsa-miR-147b, hsa-miR-664, hsa-miR-32*, hsa-miR-1269, hsa-miR-765, hsa-miR-15b, hsa-miR-495, hsa-miR-1297, hsa-miR-7-1*, hsa-miR-34c-3p, hsa-miR-767-3p, hsa-miR-190b, hsa-miR-605, hsa-miR-200c*, hsa-miR-608, hsa-miR-498, hsa-miR-1323, hsa-miR-653, hsa-miR-888, hsa-miR-568, hsa-miR-596, hsa-miR-488, hsa-miR-570, hsa-miR-105, hsa-miR-124, hsa-miR-940, hsa-miR-548d-5p, hsa-miR-34b, hsa-miR-1261, hsa-miR-222, hsa-miR-34b*, hsa-miR-1285, hsa-miR-195, hsa-miR-450b-5p, hsa-miR-610, hsa-miR-545, hsa-miR-935, hsa-miR-612, hsa-miR-27b, hsa-miR-664*, hsa-miR-548j, hsa-miR-607, hsa-miR-10b*, hsa-miR-1206, hsa-miR-200b*, hsa-miR-422a, hsa-miR-23b, hsa-miR-1263, hsa-miR-545*, hsa-miR-548a-3p, hsa-miR-1229, hsa-miR-412, hsa-miR-634, hsa-miR-136, hsa-miR-301a, hsa-miR-548h, hsa-miR-625*, hsa-miR-708*, hsa-miR-211, hsa-miR-519e*, hsa-let-7f-1*, hsa-miR-628-5p, hsa-miR-150, hsa-miR-583, hsa-miR-501-5p, hsa-miR-493, hsa-miR-224, hsa-miR-151-5p, hsa-miR-659, hsa-miR-556-5p, hsa-miR-1321, hsa-miR-138-2*, hsa-miR-130a*, hsa-miR-520h, hsa-miR-522, hsa-miR-555, hsa-miR-1827, hsa-miR-24, hsa-miR-548c-3p, hsa-miR-135a*, hsa-miR-7, hsa-miR-656, hsa-miR-454, hsa-miR-1264, hsa-miR-297, hsa-miR-548k, hsa-miR-5481, hsa-miR-424, hsa-miR-506, hsa-miR-1283, hsa-miR-548e, hsa-miR-1251, hsa-miR-137, hsa-miR-7-2*, hsa-miR-196a, hsa-miR-107, hsa-miR-188-5p, hsa-miR-15a, hsa-miR-485-3p, hsa-miR-601, hsa-miR-876-3p, hsa-miR-196b, hsa-let-7a*, hsa-miR-1272, hsa-miR-340, hsa-miR-193b*, hsa-miR-497, hsa-miR-517c, hsa-miR-649, hsa-miR-499-3p, hsa-miR-26a, hsa-miR-1275, hsa-miR-520d-5p, hsa-miR-200a*, hsa-miR-1205, hsa-miR-135b*, hsa-miR-671-5p.

Integrator complex subunit 2—INTS2—NM_{—}020748—hsa-miR-576-3p, hsa-miR-194*, hsa-miR-376b, hsa-miR-665, hsa-miR-510, hsa-miR-500, hsa-miR-130b*, hsa-let-7d, hsa-miR-766, hsa-miR-1276, hsa-miR-125b-2*, hsa-miR-195*, hsa-miR-302c*, hsa-miR-204, hsa-miR-214, hsa-miR-377, hsa-miR-1304, hsa-miR-548a-5p, hsa-miR-559, hsa-miR-27a, hsa-miR-1248, hsa-miR-335, hsa-miR-92a-2*, hsa-miR-548b-5p, hsa-miR-16-2*, hsa-miR-661, hsa-miR-31, hsa-miR-513a-3p, hsa-miR-148a*, hsa-miR-142-5p, hsa-miR-183*, hsa-miR-217, hsa-miR-1288, hsa-miR-548c-5p, hsa-miR-342-3p, hsa-miR-298, hsa-miR-508-5p, hsa-miR-455-5p, hsa-let-7e, hsa-miR-623, hsa-miR-657, hsa-miR-299-5p, hsa-miR-338-5p, hsa-miR-1296, hsa-miR-524-5p, hsa-miR-30b, hsa-miR-186, hsa-miR-23a, hsa-miR-33a*, hsa-miR-491-5p, hsa-miR-548i, hsa-miR-202, hsa-miR-494, hsa-miR-30a, hsa-miR-622, hsa-miR-548o, hsa-miR-1, hsa-miR-376a, hsa-miR-581, hsa-miR-616*, hsa-miR-552, hsa-miR-373*, hsa-miR-548f, hsa-miR-26b, hsa-let-7f, hsa-miR-590-3p, hsa-miR-1297, hsa-miR-7-1*, hsa-miR-135a, hsa-miR-146a, hsa-miR-608, hsa-miR-1323, hsa-miR-653, hsa-miR-888, hsa-miR-654-3p, hsa-miR-570, hsa-let-7g, hsa-miR-548d-5p, hsa-miR-1285, hsa-miR-20a*, hsa-miR-545, hsa-miR-452*, hsa-miR-612, hsa-miR-135b, hsa-miR-27b, hsa-miR-548j, hsa-miR-200a, hsa-miR-30d, hsa-miR-10b*, hsa-miR-629, hsa-miR-192*, hsa-let-7i, hsa-miR-23b, hsa-miR-548a-3p, hsa-miR-146b-5p, hsa-miR-526b, hsa-let-7b, hsa-miR-302d*, hsa-miR-141, hsa-let-7a, hsa-miR-22*, hsa-miR-98, hsa-miR-548h, hsa-miR-381, hsa-miR-708*, hsa-miR-211, hsa-miR-30c, hsa-miR-205, hsa-miR-17*, hsa-miR-640, hsa-miR-199a-3p, hsa-miR-30b*, hsa-miR-130a*, hsa-miR-550, hsa-miR-758, hsa-let-7c, hsa-miR-1228, hsa-miR-196a*, hsa-miR-7, hsa-miR-1264, hsa-miR-675, hsa-miR-654-5p, hsa-miR-1283, hsa-miR-187*, hsa-miR-548e, hsa-miR-199b-3p, hsa-miR-137, hsa-miR-888*, hsa-miR-206, hsa-miR-7-2*, hsa-miR-377*, hsa-miR-188-5p, hsa-miR-300, hsa-miR-632, hsa-miR-944, hsa-miR-219-2-3p, hsa-miR-346, hsa-miR-302b*, hsa-miR-433, hsa-miR-595, hsa-miR-154, hsa-miR-26a, hsa-miR-541, hsa-miR-520d-5p, hsa-miR-613, hsa-miR-340*, hsa-miR-30e.

Family with sequence similarity 152, member A—FAM152A—NM_{—}016076—hsa-miR-576-3p, hsa-miR-490-5p, hsa-miR-1266, hsa-miR-380, hsa-miR-515-5p, hsa-miR-542-3p, hsa-miR-922, hsa-miR-493*, hsa-miR-138, hsa-miR-130b*, hsa-miR-216a, hsa-miR-19b-2*, hsa-miR-1184, hsa-miR-150*, hsa-miR-1290, hsa-miR-222*, hsa-miR-1301, hsa-miR-181a, hsa-miR-551b*, hsa-miR-513b, hsa-miR-195*, hsa-miR-487a, hsa-miR-483-3p, hsa-miR-548g, hsa-miR-1289, hsa-miR-377, hsa-miR-890, hsa-miR-548a-5p, hsa-miR-559, hsa-miR-548b-5p, hsa-miR-452, hsa-miR-16-2*, hsa-miR-320c, hsa-miR-31, hsa-miR-148a*, hsa-miR-24-1*, hsa-miR-548m, hsa-miR-183*, hsa-miR-1237, hsa-miR-561, hsa-miR-541*, hsa-miR-548n, hsa-miR-217, hsa-miR-93*, hsa-miR-548c-5p, hsa-miR-19a*, hsa-miR-342-3p, hsa-miR-1287, hsa-miR-875-3p, hsa-miR-651, hsa-miR-580, hsa-miR-379, hsa-miR-600, hsa-miR-548p, hsa-miR-582-5p, hsa-miR-19b-1*, hsa-miR-524-5p, hsa-miR-30b, hsa-miR-34a*, hsa-miR-374a*, hsa-miR-23a, hsa-miR-548i, hsa-miR-606, hsa-miR-24-2*, hsa-miR-641, hsa-miR-212, hsa-miR-548d-3p, hsa-miR-646, hsa-let-7b*, hsa-miR-151-3p, hsa-miR-590-5p, hsa-miR-145*, hsa-miR-1281, hsa-miR-411, hsa-miR-30a, hsa-miR-622, hsa-miR-324-3p, hsa-miR-581, hsa-miR-1207-3p, hsa-miR-548f, hsa-miR-569, hsa-miR-664, hsa-miR-1324, hsa-miR-320d, hsa-miR-188-3p, hsa-miR-590-3p, hsa-miR-502-5p, hsa-miR-495, hsa-miR-132, hsa-miR-7-1*, hsa-miR-589*, hsa-miR-1253, hsa-miR-1200, hsa-miR-643, hsa-miR-376a*, hsa-miR-509-3p, hsa-miR-653, hsa-miR-568, hsa-miR-592, hsa-miR-570, hsa-miR-105, hsa-miR-155, hsa-miR-548d-5p, hsa-miR-34b, hsa-miR-1261, hsa-miR-184, hsa-miR-768-3p, hsa-miR-148b*, hsa-miR-125a-3p, hsa-miR-198, hsa-miR-181d, hsa-miR-935, hsa-miR-9*, hsa-miR-664*, hsa-miR-548j, hsa-miR-200a, hsa-miR-30d, hsa-miR-10b*, hsa-miR-629*, hsa-miR-629, hsa-miR-155*, hsa-miR-518c*, hsa-miR-548b-3p, hsa-miR-942, hsa-miR-23b, hsa-miR-101, hsa-miR-23a*, hsa-miR-545*, hsa-miR-1257, hsa-miR-548a-3p, hsa-miR-23b*, hsa-miR-412, hsa-miR-567, hsa-miR-302d*, hsa-miR-141, hsa-miR-223, hsa-miR-203, hsa-miR-548h, hsa-miR-625*, hsa-miR-381, hsa-miR-519e*, hsa-let-7f-1*, hsa-miR-1300, hsa-miR-628-5p, hsa-miR-30c, hsa-miR-181c, hsa-miR-224, hsa-miR-659, hsa-miR-138-2*, hsa-miR-130a*, hsa-miR-320a, hsa-miR-1299, hsa-miR-7, hsa-miR-320b, hsa-miR-297, hsa-miR-5481, hsa-miR-626, hsa-miR-181b, hsa-miR-548e, hsa-miR-21, hsa-miR-137, hsa-miR-892a, hsa-miR-7-2*, hsa-miR-300, hsa-miR-485-3p, hsa-miR-409-3p, hsa-miR-126*, hsa-let-7a*, hsa-miR-340, hsa-miR-1197, hsa-miR-218, hsa-miR-302b*, hsa-miR-1183, hsa-miR-223*, hsa-miR-1275, hsa-miR-520d-5p, hsa-miR-154*, hsa-miR-30e.

Integrator complex subunit 8—INTS8—NM_{—}017864—hsa-miR-200b, hsa-miR-493*, hsa-miR-204, hsa-miR-28-3p, hsa-miR-452, hsa-miR-190, hsa-miR-216b, hsa-miR-374a*, hsa-miR-30a*, hsa-miR-200c, hsa-miR-190b, hsa-miR-361-5p, hsa-miR-30d*, hsa-miR-30e*, hsa-miR-21*, hsa-miR-429, hsa-miR-570, hsa-miR-629*, hsa-miR-708*, hsa-miR-211, hsa-miR-1284, hsa-miR-1264, hsa-miR-16-1*.

THUMP domain containing 3—THUMPD3—NM_{—}015453—hsa-miR-557, hsa-miR-665, hsa-miR-509-3-5p, hsa-miR-34c-5p, hsa-miR-380, hsa-miR-515-5p, hsa-miR-216a, hsa-miR-19b-2*, hsa-miR-1276, hsa-miR-133a, hsa-miR-374b, hsa-miR-181a, hsa-miR-377, hsa-miR-548a-5p, hsa-miR-559, hsa-miR-92a-2*, hsa-miR-363*, hsa-miR-548b-5p, hsa-miR-516a-3p, hsa-miR-320c, hsa-miR-148a*, hsa-miR-26a-1*, hsa-miR-335*, hsa-miR-507, hsa-miR-449a, hsa-miR-330-3p, hsa-miR-302a*, hsa-miR-561, hsa-miR-129-5p, hsa-miR-548n, hsa-miR-548c-5p, hsa-miR-19a*, hsa-miR-938, hsa-miR-130b, hsa-miR-370, hsa-miR-875-3p, hsa-miR-19b-1*, hsa-miR-186, hsa-miR-23a, hsa-miR-301b, hsa-miR-449b, hsa-miR-1305, hsa-miR-337-3p, hsa-miR-491-5p, hsa-miR-548i, hsa-miR-606, hsa-miR-410, hsa-miR-421, hsa-miR-641, hsa-miR-548d-3p, hsa-miR-516b*, hsa-miR-646, hsa-miR-329, hsa-miR-622, hsa-miR-548o, hsa-miR-130a, hsa-miR-26b, hsa-miR-664, hsa-miR-573, hsa-miR-320d, hsa-miR-188-3p, hsa-miR-599, hsa-miR-29c, hsa-miR-513c, hsa-miR-1297, hsa-miR-7-1*, hsa-miR-1200, hsa-miR-34a, hsa-miR-29b, hsa-miR-26a-2*, hsa-miR-1243, hsa-miR-518a-5p, hsa-miR-608, hsa-miR-144, hsa-miR-1323, hsa-miR-384, hsa-miR-802, hsa-miR-21*, hsa-miR-29a, hsa-miR-570, hsa-miR-155, hsa-miR-548d-5p, hsa-miR-325, hsa-miR-768-3p, hsa-miR-125a-3p, hsa-miR-15a*, hsa-miR-181d, hsa-miR-9*, hsa-miR-514, hsa-miR-664*, hsa-miR-548j, hsa-miR-323-3p, hsa-miR-629*, hsa-miR-383, hsa-miR-29b-2*, hsa-miR-23b, hsa-miR-362-3p, hsa-miR-133b, hsa-miR-301a, hsa-miR-203, hsa-miR-548h, hsa-miR-597, hsa-miR-519e*, hsa-miR-150, hsa-miR-181c, hsa-miR-205, hsa-miR-1284, hsa-miR-527, hsa-miR-17*, hsa-miR-640, hsa-miR-18a*, hsa-miR-130a*, hsa-miR-550, hsa-miR-320a, hsa-miR-522, hsa-miR-1228, hsa-miR-106a*, hsa-miR-320b, hsa-miR-454, hsa-miR-297, hsa-miR-5481, hsa-miR-181b, hsa-miR-1251, hsa-miR-7-2*, hsa-miR-374a, hsa-miR-1244, hsa-miR-1272, hsa-miR-509-5p, hsa-miR-767-5p, hsa-miR-369-3p, hsa-let-7f-2*, hsa-miR-16-1*, hsa-miR-223*, hsa-miR-595, hsa-miR-26a, hsa-miR-512-5p.

Centrosomal protein 350 kDa—CEP350—NM_{—}014810—hsa-miR-140-5p, hsa-miR-665, hsa-miR-200b, hsa-miR-877, hsa-miR-32, hsa-miR-1254, hsa-miR-20b*, hsa-miR-493*, hsa-miR-648, hsa-miR-219-1-3p, hsa-miR-875-5p, hsa-miR-650, hsa-miR-9, hsa-miR-133a, hsa-miR-616, hsa-miR-222*, hsa-miR-374b, hsa-miR-181 a, hsa-miR-519b-3p, hsa-miR-513b, hsa-miR-195*, hsa-miR-487a, hsa-let-7g*, hsa-miR-335, hsa-miR-127-5p, hsa-miR-452, hsa-miR-16-2*, hsa-miR-511, hsa-miR-26a-1*, hsa-miR-335*, hsa-miR-29b-1*, hsa-miR-183*, hsa-miR-302a*, hsa-miR-561, hsa-miR-191, hsa-miR-519c-3p, hsa-miR-19b, hsa-miR-129-5p, hsa-miR-194, hsa-miR-93*, hsa-let-7c*, hsa-miR-769-5p, hsa-miR-603, hsa-miR-519a, hsa-miR-508-5p, hsa-miR-92b, hsa-miR-587, hsa-miR-1303, hsa-miR-1207-5p, hsa-miR-875-3p, hsa-miR-216b, hsa-miR-103, hsa-miR-1252, hsa-miR-630, hsa-miR-660, hsa-miR-1246, hsa-miR-299-5p, hsa-miR-576-5p, hsa-miR-548p, hsa-miR-586, hsa-miR-367, hsa-miR-338-5p, hsa-miR-524-5p, hsa-miR-30b, hsa-miR-543, hsa-miR-25, hsa-miR-186, hsa-miR-23a, hsa-miR-1305, hsa-miR-10a, hsa-miR-33a*, hsa-miR-302f, hsa-miR-1201, hsa-miR-641, hsa-miR-646, hsa-miR-153, hsa-miR-582-3p, hsa-miR-30a*, hsa-miR-30a, hsa-miR-29a*, hsa-miR-200c, hsa-miR-1207-3p, hsa-miR-548f, hsa-miR-26b, hsa-miR-664, hsa-miR-450a, hsa-miR-624, hsa-miR-617, hsa-miR-1297, hsa-miR-7-1*, hsa-miR-767-3p, hsa-miR-1200, hsa-miR-936, hsa-miR-643, hsa-miR-26a-2*, hsa-miR-520f, hsa-miR-768-5p, hsa-miR-1243, hsa-miR-518a-5p, hsa-miR-633, hsa-miR-888, hsa-miR-30d*, hsa-miR-802, hsa-miR-654-3p, hsa-miR-30e*, hsa-miR-429, hsa-miR-940, hsa-miR-155, hsa-miR-325, hsa-miR-588, hsa-miR-125a-3p, hsa-miR-450b-5p, hsa-miR-487b, hsa-miR-198, hsa-miR-20a*, hsa-miR-181d, hsa-miR-30d, hsa-miR-629, hsa-miR-155*, hsa-miR-548b-3p, hsa-miR-942, hsa-miR-23b, hsa-miR-101, hsa-miR-545*, hsa-miR-609, hsa-miR-548a-3p, hsa-miR-1255a, hsa-miR-33b, hsa-miR-516b, hsa-miR-133b, hsa-miR-567, hsa-miR-328, hsa-miR-330-5p, hsa-miR-139-5p, hsa-miR-92a, hsa-miR-1300, hsa-miR-30c, hsa-miR-181c, hsa-miR-181a-2*, hsa-miR-583, hsa-miR-527, hsa-miR-640, hsa-miR-130a*, hsa-miR-10b, hsa-miR-522, hsa-miR-486-5p, hsa-miR-202*, hsa-miR-1827, hsa-miR-1299, hsa-miR-135a*, hsa-miR-106a*, hsa-miR-297, hsa-miR-5481, hsa-miR-181b, hsa-miR-1283, hsa-miR-187*, hsa-miR-548e, hsa-miR-655, hsa-miR-485-5p, hsa-miR-577, hsa-miR-122, hsa-miR-7-2*, hsa-miR-33a, hsa-miR-196a, hsa-miR-107, hsa-miR-19a, hsa-miR-632, hsa-miR-374a, hsa-miR-627, hsa-miR-455-3p, hsa-miR-196b, hsa-miR-363, hsa-miR-944, hsa-miR-574-5p, hsa-miR-1265, hsa-miR-499-5p, hsa-miR-193b*, hsa-miR-140-3p, hsa-miR-887, hsa-miR-369-3p, hsa-miR-326, hsa-miR-497*, hsa-miR-223*, hsa-miR-499-3p, hsa-miR-26a, hsa-miR-1255b, hsa-miR-520d-5p, hsa-miR-135b*, hsa-miR-154*, hsa-miR-512-5p, hsa-miR-30e.

Ewing tumor-associated antigen 1—ETAA1—NM_{—}019002—hsa-miR-374b, hsa-miR-101*, hsa-miR-551b*, hsa-miR-513a-3p, hsa-miR-142-5p, hsa-miR-1277, hsa-miR-374a*, hsa-miR-33a*, hsa-miR-302f, hsa-miR-606, hsa-miR-410, hsa-miR-421, hsa-miR-641, hsa-miR-548o, hsa-miR-1323, hsa-miR-568, hsa-miR-607, hsa-miR-539, hsa-miR-889, hsa-miR-1264, hsa-miR-374a.

WD repeat domain 68—WDR68—NM_{—}005828—hsa-miR-194*, hsa-miR-665, hsa-miR-637, hsa-miR-200b, hsa-miR-509-3-5p, hsa-miR-520b, hsa-miR-490-5p, hsa-miR-34c-5p, hsa-miR-642, hsa-miR-645, hsa-miR-16, hsa-miR-515-5p, hsa-miR-380*, hsa-miR-27b*, hsa-miR-141*, hsa-miR-922, hsa-miR-625, hsa-miR-138, hsa-miR-216a, hsa-miR-562, hsa-miR-766, hsa-miR-1276, hsa-miR-1184, hsa-miR-520g, hsa-miR-374b, hsa-miR-1301, hsa-miR-302c*, hsa-miR-125a-5p, hsa-miR-221, hsa-miR-204, hsa-miR-1289, hsa-miR-563, hsa-miR-214, hsa-miR-532-3p, hsa-miR-377, hsa-miR-549, hsa-miR-1304, hsa-miR-548a-5p, hsa-miR-376c, hsa-miR-647, hsa-miR-559, hsa-miR-27a, hsa-miR-1248, hsa-miR-335, hsa-miR-138-1*, hsa-miR-92a-2*, hsa-miR-363*, hsa-miR-548b-5p, hsa-miR-516a-3p, hsa-miR-452, hsa-miR-575, hsa-miR-520a-3p, hsa-miR-661, hsa-miR-544, hsa-miR-486-3p, hsa-miR-335*, hsa-miR-449a, hsa-miR-330-3p, hsa-miR-28-5p, hsa-miR-561, hsa-miR-19b, hsa-miR-129-5p, hsa-miR-194, hsa-miR-892b, hsa-miR-146b-3p, hsa-miR-302e, hsa-miR-548c-5p, hsa-miR-603, hsa-miR-1825, hsa-miR-128, hsa-miR-134, hsa-miR-1308, hsa-miR-130b, hsa-miR-587, hsa-miR-875-3p, hsa-miR-1252, hsa-miR-1182, hsa-miR-660, hsa-miR-148a, hsa-miR-600, hsa-miR-576-5p, hsa-miR-548p, hsa-miR-582-5p, hsa-miR-25*, hsa-miR-524-5p, hsa-miR-30b, hsa-miR-143*, hsa-miR-186, hsa-miR-365, hsa-miR-301b, hsa-miR-449b, hsa-miR-1236, hsa-miR-33a*, hsa-miR-302c, hsa-miR-491-5p, hsa-miR-1201, hsa-miR-548i, hsa-miR-520a-5p, hsa-miR-410, hsa-miR-421, hsa-miR-641, hsa-miR-548d-3p, hsa-miR-516b*, hsa-miR-646, hsa-let-7b*, hsa-miR-151-3p, hsa-miR-708, hsa-miR-146a*, hsa-miR-590-5p, hsa-miR-525-5p, hsa-miR-494, hsa-miR-30a*, hsa-miR-371-5p, hsa-miR-30a, hsa-miR-489, hsa-miR-622, hsa-miR-324-3p, hsa-miR-373, hsa-miR-200c, hsa-miR-1207-3p, hsa-miR-130a, hsa-miR-616*, hsa-miR-552, hsa-miR-148b, hsa-miR-373*, hsa-miR-220c, hsa-miR-1286, hsa-miR-720, hsa-miR-573, hsa-miR-302d, hsa-miR-15b, hsa-miR-1324, hsa-miR-188-3p, hsa-miR-617, hsa-miR-590-3p, hsa-miR-495, hsa-miR-29c, hsa-miR-513c, hsa-miR-7-1*, hsa-miR-589, hsa-miR-635, hsa-miR-767-3p, hsa-miR-589*, hsa-miR-1253, hsa-miR-1200, hsa-miR-34a, hsa-miR-29b, hsa-miR-135a, hsa-miR-193a-3p, hsa-miR-605, hsa-miR-374b*, hsa-miR-186*, hsa-miR-873, hsa-miR-520c-3p, hsa-miR-518a-5p, hsa-miR-608, hsa-miR-27a*, hsa-miR-770-5p, hsa-miR-18b, hsa-miR-1226, hsa-miR-185*, hsa-miR-1262, hsa-miR-144, hsa-miR-125b, hsa-miR-220b, hsa-miR-505*, hsa-miR-302a, hsa-miR-1224-5p, hsa-miR-888, hsa-miR-30d*, hsa-miR-654-3p, hsa-miR-30e*, hsa-miR-122*, hsa-miR-429, hsa-miR-29a, hsa-miR-624*, hsa-miR-488, hsa-miR-570, hsa-miR-105, hsa-miR-124, hsa-miR-940, hsa-miR-155, hsa-miR-548d-5p, hsa-miR-34b, hsa-miR-588, hsa-miR-222, hsa-miR-1270, hsa-miR-769-3p, hsa-miR-768-3p, hsa-miR-34b*, hsa-miR-148b*, hsa-miR-125a-3p, hsa-miR-1285, hsa-miR-450b-3p, hsa-miR-520d-3p, hsa-miR-18b*, hsa-miR-195, hsa-miR-450b-5p, hsa-miR-144*, hsa-miR-545, hsa-miR-504, hsa-miR-935, hsa-miR-612, hsa-miR-135b, hsa-miR-27b, hsa-miR-299-3p, hsa-miR-664*, hsa-miR-636, hsa-miR-548j, hsa-miR-520e, hsa-miR-30d, hsa-miR-29b-2*, hsa-miR-155*, hsa-miR-193b, hsa-miR-139-3p, hsa-miR-491-3p, hsa-miR-1260, hsa-miR-152, hsa-miR-101, hsa-miR-1274a, hsa-miR-545*, hsa-miR-1229, hsa-miR-634, hsa-miR-516b, hsa-miR-526b, hsa-miR-149*, hsa-miR-620, hsa-miR-136, hsa-miR-328, hsa-miR-330-5p, hsa-miR-301a, hsa-miR-223, hsa-miR-548h, hsa-miR-492, hsa-miR-211, hsa-miR-519e*, hsa-let-7f-1*, hsa-miR-432*, hsa-miR-150, hsa-miR-30c, hsa-miR-181a-2*, hsa-miR-583, hsa-miR-493, hsa-miR-18a, hsa-miR-1284, hsa-miR-527, hsa-miR-185, hsa-miR-1321, hsa-miR-631, hsa-miR-138-2*, hsa-miR-877*, hsa-miR-18a*, hsa-miR-520h, hsa-miR-555, hsa-miR-431, hsa-miR-202*, hsa-miR-1827, hsa-miR-1299, hsa-miR-106a*, hsa-miR-7, hsa-miR-382, hsa-miR-454, hsa-miR-5481, hsa-miR-424, hsa-miR-506, hsa-miR-302b, hsa-miR-1283, hsa-miR-187*, hsa-miR-21, hsa-miR-485-5p, hsa-miR-614, hsa-miR-1278, hsa-miR-331-3p, hsa-miR-888*, hsa-miR-924, hsa-miR-7-2*, hsa-miR-19a, hsa-miR-15a, hsa-miR-374a, hsa-miR-627, hsa-miR-876-3p, hsa-miR-455-3p, hsa-miR-126*, hsa-let-7a*, hsa-miR-509-5p, hsa-miR-944, hsa-miR-340, hsa-miR-193b*, hsa-miR-497, hsa-miR-296-3p, hsa-let-7f-2*, hsa-miR-1197, hsa-miR-346, hsa-miR-218, hsa-miR-372, hsa-miR-326, hsa-miR-1183, hsa-miR-505, hsa-miR-499-3p, hsa-miR-662, hsa-miR-1234, hsa-miR-1275, hsa-miR-1250, hsa-miR-520d-5p, hsa-miR-1205, hsa-miR-1302, hsa-miR-425*, hsa-miR-512-5p, hsa-miR-30e.

Viral DNA polymerase-transactivated protein 6—DNAPTP6—Variant 1, Variant 2, Variant 3 and Variant 4—NM_{—}015535, NM_{—}001100422, NM_{—}001100423 and NM_{—}001100424—hsa-miR-140-5p, hsa-miR-557, hsa-miR-665, hsa-miR-200b, hsa-miR-1266, hsa-miR-518a-3p, hsa-miR-32, hsa-miR-1254, hsa-miR-645, hsa-miR-380, hsa-miR-448, hsa-miR-500, hsa-miR-1180, hsa-miR-20b*, hsa-miR-922, hsa-miR-219-1-3p, hsa-miR-562, hsa-miR-766, hsa-miR-616, hsa-miR-181a, hsa-miR-551b*, hsa-miR-454*, hsa-miR-221, hsa-miR-548g, hsa-miR-204, hsa-miR-96, hsa-miR-1179, hsa-miR-490-3p, hsa-miR-1267, hsa-miR-920, hsa-miR-92a-2*, hsa-miR-363*, hsa-miR-127-5p, hsa-miR-452, hsa-miR-661, hsa-miR-507, hsa-miR-548m, hsa-miR-378, hsa-miR-330-3p, hsa-miR-1226*, hsa-miR-190, hsa-miR-561, hsa-miR-191, hsa-miR-19b, hsa-miR-541*, hsa-miR-548n, hsa-miR-146b-3p, hsa-miR-1288, hsa-miR-658, hsa-miR-769-5p, hsa-miR-886-3p, hsa-miR-134, hsa-miR-1268, hsa-miR-508-5p, hsa-miR-92b, hsa-miR-130b, hsa-miR-1207-5p, hsa-miR-216b, hsa-miR-103, hsa-miR-1182, hsa-miR-580, hsa-miR-1246, hsa-miR-548p, hsa-miR-876-5p, hsa-miR-367, hsa-miR-25*, hsa-miR-30b, hsa-miR-25, hsa-miR-186, hsa-miR-23a, hsa-miR-301b, hsa-miR-1236, hsa-miR-10a, hsa-miR-33a*, hsa-miR-491-5p, hsa-miR-548d-3p, hsa-miR-30a*, hsa-miR-30a, hsa-miR-489, hsa-miR-622, hsa-miR-548o, hsa-miR-1, hsa-miR-1227, hsa-miR-200c, hsa-miR-130a, hsa-miR-616*, hsa-miR-874, hsa-miR-373*, hsa-miR-548f, hsa-miR-569, hsa-miR-518b, hsa-miR-664, hsa-miR-484, hsa-miR-1269, hsa-miR-1202, hsa-miR-1324, hsa-miR-96*, hsa-miR-1271, hsa-miR-589, hsa-miR-939, hsa-miR-767-3p, hsa-miR-190b, hsa-miR-885-3p, hsa-miR-921, hsa-miR-135a, hsa-miR-579, hsa-miR-1259, hsa-miR-374b*, hsa-miR-873, hsa-miR-518a-5p, hsa-miR-608, hsa-miR-509-3p, hsa-miR-1262, hsa-miR-144, hsa-miR-1323, hsa-miR-653, hsa-miR-30d*, hsa-miR-802, hsa-miR-654-3p, hsa-miR-30e*, hsa-miR-21*, hsa-miR-429, hsa-miR-488, hsa-miR-105, hsa-miR-508-3p, hsa-miR-940, hsa-miR-34b, hsa-miR-588, hsa-miR-222, hsa-miR-768-3p, hsa-miR-125a-3p, hsa-miR-1285, hsa-miR-450b-5p, hsa-miR-610, hsa-miR-92a-1*, hsa-miR-20a*, hsa-miR-545, hsa-miR-181d, hsa-miR-504, hsa-miR-612, hsa-miR-135b, hsa-miR-514, hsa-miR-664*, hsa-miR-607, hsa-miR-30d, hsa-miR-554, hsa-miR-518c*, hsa-miR-942, hsa-miR-422a, hsa-miR-23b, hsa-miR-101, hsa-miR-1274a, hsa-miR-539, hsa-miR-548a-3p, hsa-miR-634, hsa-miR-149*, hsa-miR-328, hsa-miR-330-5p, hsa-miR-139-5p, hsa-miR-301a, hsa-miR-518c, hsa-miR-92a, hsa-miR-492, hsa-miR-211, hsa-miR-593*, hsa-miR-1300, hsa-miR-30c, hsa-miR-1273, hsa-miR-181c, hsa-miR-181 a-2*, hsa-miR-221*, hsa-miR-527, hsa-miR-378*, hsa-miR-1321, hsa-miR-631, hsa-miR-18a*, hsa-miR-30b*, hsa-miR-130a*, hsa-miR-550, hsa-miR-1225-5p, hsa-miR-10b, hsa-miR-431, hsa-miR-339-5p, hsa-miR-486-5p, hsa-miR-1228, hsa-miR-1827, hsa-miR-24, hsa-miR-518f, hsa-miR-663b, hsa-miR-106a*, hsa-miR-182, hsa-miR-454, hsa-miR-1264, hsa-miR-1274b, hsa-miR-654-5p, hsa-miR-181b, hsa-miR-1283, hsa-miR-187*, hsa-miR-548e, hsa-miR-655, hsa-miR-518d-3p, hsa-miR-485-5p, hsa-miR-888*, hsa-miR-924, hsa-miR-206, hsa-miR-122, hsa-miR-377*, hsa-miR-107, hsa-miR-19a, hsa-miR-425, hsa-miR-627, hsa-miR-409-3p, hsa-miR-126*, hsa-miR-363, hsa-miR-767-5p, hsa-miR-193b*, hsa-miR-371-3p, hsa-miR-16-1*, hsa-miR-326, hsa-miR-433, hsa-miR-505, hsa-miR-499-3p, hsa-miR-1234, hsa-miR-541, hsa-miR-891b, hsa-miR-613, hsa-miR-30e.

Chromosome 7 open reading frame 25-C7orf25—Variant 1 and Variant 2—NM_{—}001099858 and NM_{—}024054—hsa-miR-32, hsa-miR-9, hsa-miR-454*, hsa-miR-29b-1*, hsa-miR-92b, hsa-miR-367, hsa-miR-25, hsa-miR-618, hsa-miR-1324, hsa-miR-374b*, hsa-miR-1260, hsa-miR-942, hsa-miR-203, hsa-miR-92a, hsa-miR-625*, hsa-miR-30b*, hsa-miR-106a*, hsa-miR-7, hsa-miR-5481, hsa-miR-488*, hsa-miR-363, hsa-miR-218, hsa-miR-154.

Chromosome 2 open reading frame 60—C2orf60—NM_{—}001039693—hsa-miR-576-3p, hsa-miR-376b, hsa-miR-517a, hsa-miR-557, hsa-miR-502-3p, hsa-miR-200b, hsa-miR-509-3-5p, hsa-miR-520b, hsa-miR-1266, hsa-miR-645, hsa-miR-380, hsa-miR-16, hsa-miR-448, hsa-miR-625, hsa-miR-493*, hsa-miR-130b*, hsa-miR-19b-2*, hsa-miR-650, hsa-miR-1276, hsa-miR-1184, hsa-miR-520g, hsa-miR-374b, hsa-miR-101*, hsa-miR-1301, hsa-miR-181a, hsa-miR-483-3p, hsa-miR-548g, hsa-miR-28-3p, hsa-miR-1179, hsa-miR-377, hsa-miR-490-3p, hsa-miR-92a-2*, hsa-miR-516a-3p, hsa-miR-452, hsa-miR-575, hsa-miR-320c, hsa-miR-520a-3p, hsa-miR-511, hsa-miR-15b*, hsa-miR-31, hsa-miR-148a*, hsa-miR-379*, hsa-miR-335*, hsa-miR-507, hsa-miR-29b-1*, hsa-miR-548m, hsa-miR-28-5p, hsa-miR-129-5p, hsa-miR-548n, hsa-miR-892b, hsa-miR-885-5p, hsa-miR-302e, hsa-miR-769-5p, hsa-miR-19a*, hsa-miR-134, hsa-miR-508-5p, hsa-miR-455-5p, hsa-miR-1303, hsa-miR-216b, hsa-miR-1182, hsa-miR-651, hsa-miR-630, hsa-miR-148a, hsa-miR-1246, hsa-miR-576-5p, hsa-miR-582-5p, hsa-miR-19b-1*, hsa-miR-524-5p, hsa-miR-143*, hsa-miR-543, hsa-miR-1236, hsa-miR-337-3p, hsa-miR-10a, hsa-miR-33a*, hsa-miR-302c, hsa-miR-548d-3p, hsa-miR-516b*, hsa-miR-646, hsa-miR-329, hsa-miR-708, hsa-miR-548o, hsa-miR-376a, hsa-miR-373, hsa-miR-200c, hsa-miR-145, hsa-miR-616*, hsa-miR-552, hsa-miR-148b, hsa-miR-373*, hsa-miR-548f, hsa-miR-26b, hsa-miR-569, hsa-miR-664, hsa-miR-484, hsa-miR-302d, hsa-miR-624, hsa-miR-296-5p, hsa-miR-15b, hsa-miR-320d, hsa-miR-590-3p, hsa-miR-495, hsa-miR-29c, hsa-miR-1297, hsa-miR-589*, hsa-miR-1200, hsa-miR-643, hsa-miR-29b, hsa-miR-135a, hsa-miR-579, hsa-miR-411*, hsa-miR-361-5p, hsa-miR-605, hsa-miR-768-5p, hsa-miR-376a*, hsa-miR-186*, hsa-miR-147, hsa-miR-520c-3p, hsa-miR-608, hsa-miR-498, hsa-miR-517b, hsa-miR-1262, hsa-miR-1323, hsa-miR-220b, hsa-miR-302a, hsa-miR-654-3p, hsa-miR-568, hsa-miR-21*, hsa-miR-429, hsa-miR-29a, hsa-miR-488, hsa-miR-1294, hsa-miR-432, hsa-miR-210, hsa-miR-1270, hsa-miR-768-3p, hsa-miR-148b*, hsa-miR-512-3p, hsa-miR-520d-3p, hsa-miR-18b*, hsa-miR-195, hsa-miR-331-5p, hsa-miR-519d, hsa-miR-92a-1*, hsa-miR-20a*, hsa-miR-452*, hsa-miR-181d, hsa-miR-135b, hsa-miR-323-3p, hsa-miR-607, hsa-miR-520e, hsa-miR-200a, hsa-miR-93, hsa-miR-383, hsa-miR-29b-2*, hsa-miR-192*, hsa-miR-518c*, hsa-miR-491-3p, hsa-miR-152, hsa-miR-1322, hsa-miR-23a*, hsa-miR-362-3p, hsa-miR-545*, hsa-miR-539, hsa-miR-548a-3p, hsa-miR-23b*, hsa-miR-33b, hsa-miR-149*, hsa-miR-620, hsa-miR-302d*, hsa-miR-136, hsa-miR-141, hsa-miR-526b*, hsa-miR-203, hsa-miR-492, hsa-miR-708*, hsa-miR-744*, hsa-miR-432*, hsa-miR-1300, hsa-miR-150, hsa-miR-181c, hsa-miR-501-5p, hsa-miR-224, hsa-miR-20b, hsa-miR-378*, hsa-miR-640, hsa-miR-1224-3p, hsa-miR-106a, hsa-miR-1245, hsa-miR-877*, hsa-miR-149, hsa-miR-18a*, hsa-miR-483-5p, hsa-miR-550, hsa-miR-520h, hsa-miR-320a, hsa-miR-10b, hsa-miR-24, hsa-miR-17, hsa-miR-500*, hsa-miR-656, hsa-miR-182, hsa-miR-320b, hsa-miR-345, hsa-miR-548l, hsa-miR-424, hsa-miR-302b, hsa-miR-181b, hsa-miR-1283, hsa-miR-548e, hsa-miR-655, hsa-miR-1278, hsa-miR-577, hsa-miR-33a, hsa-miR-196a, hsa-miR-15a, hsa-miR-374a, hsa-miR-20a, hsa-miR-425, hsa-miR-409-3p, hsa-miR-183, hsa-miR-196b, hsa-miR-509-5p, hsa-miR-944, hsa-miR-501-3p, hsa-miR-193b*, hsa-miR-140-3p, hsa-miR-106b, hsa-miR-497, hsa-miR-369-3p, hsa-miR-517c, hsa-miR-302b*, hsa-miR-372, hsa-miR-497*, hsa-miR-154, hsa-miR-26a, hsa-miR-1275, hsa-miR-520d-5p, hsa-miR-891b.

Chromosome 17 open reading frame 71—C17orf71—NM_{—}018149—hsa-miR-200b, hsa-miR-548n, hsa-miR-892b, hsa-miR-875-3p, hsa-miR-338-5p, hsa-miR-30b, hsa-miR-548d-3p, hsa-miR-30a, hsa-miR-200c, hsa-miR-26b, hsa-miR-1297, hsa-miR-7-1*, hsa-miR-429, hsa-miR-570, hsa-miR-30d, hsa-miR-545*, hsa-miR-30c, hsa-miR-382, hsa-miR-7-2*, hsa-miR-26a, hsa-miR-30e.

Chromosome 7 open reading frame 36—C7orf36'NM_{—}020192—hsa-miR-513a-3p, hsa-miR-335*, hsa-miR-587, hsa-miR-582-5p, hsa-miR-770-5p, hsa-miR-633, hsa-miR-1258, hsa-miR-139-5p.

Elongation protein 4 homolog—ELP4—NM_{—}019040—hsa-miR-376b, hsa-miR-518d-5p, hsa-miR-519a*, hsa-miR-522*, hsa-miR-544, hsa-miR-548n, hsa-miR-1252, hsa-miR-376a, hsa-miR-517*, hsa-miR-518P, hsa-miR-148b*, hsa-miR-520c-5p, hsa-miR-539, hsa-miR-518e*, hsa-miR-593*, hsa-miR-526a, hsa-miR-523*, hsa-miR-548l, hsa-miR-519b-5p, hsa-miR-767-5p, hsa-miR-944, hsa-miR-519c-5p.

nudE nuclear distribution gene E homolog (A. nidulans)-like 1—NDEL1—Variant 1—NM_{—}001025579—hsa-miR-665, hsa-miR-22, hsa-miR-922, hsa-miR-650, hsa-miR-519b-3p, hsa-miR-302c*, hsa-miR-548g, hsa-miR-377, hsa-miR-920, hsa-miR-363*, hsa-miR-575, hsa-miR-661, hsa-miR-544, hsa-miR-486-3p, hsa-miR-519c-3p, hsa-miR-217, hsa-miR-658, hsa-miR-603, hsa-miR-519a, hsa-miR-342-3p, hsa-miR-130b, hsa-miR-619, hsa-miR-1207-5p, hsa-miR-103, hsa-miR-1203, hsa-miR-524-5p, hsa-miR-30b, hsa-miR-186, hsa-miR-301b, hsa-miR-337-3p, hsa-miR-33a*, hsa-miR-92b*, hsa-let-7b*, hsa-miR-128l, hsa-miR-30a, hsa-miR-29a*, hsa-miR-130a, hsa-miR-220c, hsa-miR-1247, hsa-miR-1202, hsa-miR-765, hsa-miR-96*, hsa-miR-7-1*, hsa-miR-1253, hsa-miR-146a, hsa-miR-768-5p, hsa-miR-608, hsa-miR-18b, hsa-miR-185*, hsa-miR-596, hsa-miR-570, hsa-miR-1270, hsa-miR-1285, hsa-miR-450b-5p, hsa-miR-519d, hsa-miR-612, hsa-miR-30d, hsa-miR-93, hsa-miR-545*, hsa-miR-146b-5p, hsa-miR-620, hsa-miR-330-5p, hsa-miR-55 1 a, hsa-miR-301a, hsa-miR-941, hsa-miR-526b*, hsa-miR-571, hsa-let-7f-1*, hsa-miR-30c, hsa-miR-18a, hsa-miR-151-5p, hsa-miR-1284, hsa-miR-20b, hsa-miR-378*, hsa-miR-1256, hsa-miR-1224-3p, hsa-miR-106a, hsa-miR-18a*, hsa-miR-30b*, hsa-miR-483-5p, hsa-miR-1225-5p, hsa-miR-1827, hsa-miR-24, hsa-miR-17, hsa-miR-106a*, hsa-miR-454, hsa-miR-345, hsa-miR-1283, hsa-miR-187*, hsa-miR-655, hsa-miR-485-5p, hsa-miR-7-2*, hsa-miR-107, hsa-miR-564, hsa-miR-20a, hsa-miR-423-5p, hsa-miR-455-3p, hsa-let-7a*, hsa-miR-1272, hsa-miR-106b, hsa-let-7f-2*, hsa-miR-1197, hsa-miR-16-1*, hsa-miR-551b, hsa-miR-326, hsa-miR-1275, hsa-miR-520d-5p, hsa-miR-1225-3p, hsa-miR-30e.

NDEL1—Variant 2—NM_{—}030808—hsa-miR-22, hsa-miR-922, hsa-miR-650, hsa-miR-519b-3p, hsa-miR-302c*, hsa-miR-548g, hsa-miR-920, hsa-miR-363*, hsa-miR-575, hsa-miR-661, hsa-miR-544, hsa-miR-486-3p, hsa-miR-519c-3p, hsa-miR-217, hsa-miR-658, hsa-miR-519a, hsa-miR-130b, hsa-miR-619, hsa-miR-1207-5p, hsa-miR-103, hsa-miR-1203, hsa-miR-524-5p, hsa-miR-30b, hsa-miR-186, hsa-miR-301b, hsa-miR-337-3p, hsa-miR-33a*, hsa-miR-92b*, hsa-let-7b*, hsa-miR-1281, hsa-miR-30a, hsa-miR-29a*, hsa-miR-130a, hsa-miR-220c, hsa-miR-1247, hsa-miR-1202, hsa-miR-765, hsa-miR-96*, hsa-miR-7-1*, hsa-miR-1253, hsa-miR-146a, hsa-miR-768-5p, hsa-miR-608, hsa-miR-18b, hsa-miR-185*, hsa-miR-596, hsa-miR-570, hsa-miR-1270, hsa-miR-1285, hsa-miR-450b-5p, hsa-miR-519d, hsa-miR-612, hsa-miR-30d, hsa-miR-93, hsa-miR-545*, hsa-miR-146b-5p, hsa-miR-620, hsa-miR-330-5p, hsa-miR-551a, hsa-miR-301a, hsa-miR-526b*, hsa-miR-571, hsa-let-7f-1*, hsa-miR-30c, hsa-miR-18a, hsa-miR-1284, hsa-miR-20b, hsa-miR-378*, hsa-miR-1256, hsa-miR-1224-3p, hsa-miR-106a, hsa-miR-18a*, hsa-miR-30b*, hsa-miR-483-5p, hsa-miR-1225-5p, hsa-miR-1827, hsa-miR-24, hsa-miR-17, hsa-miR-106a*, hsa-miR-454, hsa-miR-345, hsa-miR-1283, hsa-miR-187*, hsa-miR-655, hsa-miR-485-5p, hsa-miR-7-2*, hsa-miR-107, hsa-miR-564, hsa-miR-20a, hsa-miR-423-5p, hsa-miR-455-3p, hsa-let-7a*, hsa-miR-1272, hsa-miR-106b, hsa-let-7f-2*, hsa-miR-1197, hsa-miR-16-1*, hsa-miR-551b, hsa-miR-326, hsa-miR-1275, hsa-miR-520d-5p, hsa-miR-1225-3p, hsa-miR-30e.

Chromosome 1 open reading frame 63—C1orf63—NM_{—}020317—hsa-miR-9, hsa-miR-133a, hsa-miR-125b-2*, hsa-miR-519b-3p, hsa-miR-195*, hsa-miR-532-3p, hsa-miR-16-2*, hsa-miR-513a-3p, hsa-miR-142-5p, hsa-miR-335*, hsa-miR-519c-3p, hsa-miR-519a, hsa-miR-582-5p, hsa-miR-338-5p, hsa-miR-143*, hsa-let-7b*, hsa-miR-570, hsa-miR-155, hsa-miR-519d, hsa-miR-93, hsa-miR-1260, hsa-miR-133b, hsa-miR-526b*, hsa-let-7f-1*, hsa-miR-20b, hsa-miR-1224-3p, hsa-miR-106a, hsa-miR-17, hsa-miR-892a, hsa-miR-20a, hsa-let-7a*, hsa-miR-106b.

Family with sequence similarity 172, member A—FAM172A—NM_{—}032042—hsa-miR-140-5p, hsa-miR-1266, hsa-miR-32, hsa-miR-642, hsa-miR-16, hsa-miR-22, hsa-miR-625, hsa-miR-1276, hsa-miR-125b-2*, hsa-miR-150*, hsa-miR-1290, hsa-miR-374b, hsa-miR-513b, hsa-miR-302c*, hsa-miR-483-3p, hsa-miR-548g, hsa-miR-1289, hsa-miR-668, hsa-miR-548a-5p, hsa-miR-559, hsa-miR-27a, hsa-miR-138-1*, hsa-miR-548b-5p, hsa-miR-143, hsa-miR-548m, hsa-miR-330-3p, hsa-miR-541*, hsa-miR-548n, hsa-miR-93*, hsa-miR-548c-5p, hsa-miR-886-3p, hsa-miR-603, hsa-miR-128, hsa-miR-298, hsa-miR-508-5p, hsa-miR-92b, hsa-miR-1207-5p, hsa-miR-1287, hsa-miR-875-3p, hsa-miR-216b, hsa-miR-1252, hsa-miR-299-5p, hsa-miR-548p, hsa-miR-367, hsa-miR-615-3p, hsa-miR-524-5p, hsa-miR-25, hsa-miR-374a*, hsa-miR-186, hsa-miR-337-3p, hsa-miR-33a*, hsa-miR-548i, hsa-miR-212, hsa-miR-548d-3p, hsa-miR-548o, hsa-miR-1, hsa-miR-581, hsa-miR-145, hsa-miR-26b, hsa-miR-32*, hsa-miR-1269, hsa-miR-15b, hsa-miR-96*, hsa-miR-590-3p, hsa-miR-495, hsa-miR-132, hsa-miR-1297, hsa-miR-589*, hsa-miR-1253, hsa-miR-885-3p, hsa-miR-643, hsa-miR-193a-3p, hsa-miR-147, hsa-miR-185*, hsa-miR-1323, hsa-miR-323-5p, hsa-miR-653, hsa-miR-802, hsa-miR-503, hsa-miR-570, hsa-miR-940, hsa-miR-548d-5p, hsa-miR-588, hsa-miR-1261, hsa-miR-195, hsa-miR-450b-5p, hsa-miR-545, hsa-miR-504, hsa-miR-27b, hsa-miR-636, hsa-miR-548j, hsa-miR-323-3p, hsa-miR-607, hsa-miR-200a, hsa-miR-578, hsa-miR-10b*, hsa-miR-629, hsa-miR-383, hsa-miR-155*, hsa-miR-193b, hsa-miR-491-3p, hsa-miR-942, hsa-miR-101, hsa-miR-572, hsa-miR-330-5p, hsa-miR-141, hsa-miR-548h, hsa-miR-92a, hsa-miR-625*, hsa-miR-760, hsa-miR-583, hsa-miR-185, hsa-miR-378*, hsa-miR-100*, hsa-miR-199a-3p, hsa-miR-1225-5p, hsa-miR-197, hsa-miR-1228, hsa-miR-1827, hsa-miR-1299, hsa-miR-548c-3p, hsa-miR-1279, hsa-miR-297, hsa-miR-548l, hsa-miR-424, hsa-miR-654-5p, hsa-miR-199b-3p, hsa-miR-137, hsa-miR-206, hsa-miR-122, hsa-miR-15a, hsa-miR-374a, hsa-miR-1244, hsa-miR-423-5p, hsa-miR-126*, hsa-miR-363, hsa-miR-574-5p, hsa-miR-499-5p, hsa-miR-193b*, hsa-miR-497, hsa-miR-369-3p, hsa-let-7f-2*, hsa-miR-218, hsa-miR-649, hsa-miR-326, hsa-miR-1183, hsa-miR-505, hsa-miR-595, hsa-miR-26a, hsa-miR-541, hsa-miR-520d-5p, hsa-miR-558, hsa-miR-135b*, hsa-miR-613.

Family with sequence similarity 53, member C—FAM53C—Variant 1 and Variant 2—NM_{—}001135647 and NM_{—}016605—hsa-miR-665, hsa-miR-518d-5p, hsa-miR-637, hsa-miR-510, hsa-miR-1266, hsa-miR-1254, hsa-miR-642, hsa-miR-519a*, hsa-miR-500, hsa-miR-522*, hsa-miR-22, hsa-miR-922, hsa-miR-562, hsa-miR-650, hsa-miR-125b-2*, hsa-miR-1290, hsa-miR-222*, hsa-miR-513b, hsa-miR-644, hsa-miR-125a-5p, hsa-miR-96, hsa-miR-28-3p, hsa-miR-214, hsa-miR-377, hsa-miR-218-1*, hsa-miR-890, hsa-miR-548a-5p, hsa-miR-559, hsa-miR-27a, hsa-miR-1248, hsa-miR-92a-2*, hsa-miR-548b-5p, hsa-miR-320c, hsa-miR-661, hsa-miR-1226*, hsa-miR-302a*, hsa-miR-561, hsa-miR-129-5p, hsa-miR-548n, hsa-miR-769-5p, hsa-miR-548c-5p, hsa-miR-886-3p, hsa-miR-603, hsa-miR-342-3p, hsa-miR-370, hsa-miR-199a-5p, hsa-miR-1207-5p, hsa-miR-1287, hsa-miR-875-3p, hsa-miR-1182, hsa-miR-1246, hsa-miR-299-5p, hsa-miR-576-5p, hsa-miR-548p, hsa-miR-586, hsa-miR-1296, hsa-miR-365, hsa-miR-618, hsa-miR-491-5p, hsa-miR-548i, hsa-miR-520a-5p, hsa-miR-641, hsa-miR-329, hsa-miR-525-5p, hsa-miR-622, hsa-miR-874, hsa-miR-220c, hsa-miR-484, hsa-miR-1202, hsa-miR-765, hsa-miR-320d, hsa-miR-1271, hsa-miR-29c, hsa-miR-513c, hsa-miR-1253, hsa-miR-936, hsa-miR-29b, hsa-miR-193a-3p, hsa-miR-147, hsa-miR-1233, hsa-miR-608, hsa-miR-1226, hsa-miR-185*, hsa-miR-125b, hsa-miR-886-5p, hsa-miR-654-3p, hsa-miR-29a, hsa-miR-570, hsa-miR-105, hsa-miR-517*, hsa-miR-940, hsa-miR-548d-5p, hsa-miR-34b, hsa-miR-588, hsa-miR-1261, hsa-miR-518f*, hsa-miR-148b*, hsa-miR-1285, hsa-miR-512-3p, hsa-miR-20a*, hsa-miR-612, hsa-miR-27b, hsa-miR-548j, hsa-miR-607, hsa-miR-520c-5p, hsa-miR-629*, hsa-miR-29b-2*, hsa-miR-193b, hsa-miR-518c*, hsa-miR-362-3p, hsa-miR-1255a, hsa-miR-33b, hsa-miR-516b, hsa-miR-149*, hsa-miR-330-5p, hsa-miR-548h, hsa-miR-361-3p, hsa-miR-518e*, hsa-miR-593*, hsa-miR-150, hsa-miR-526a, hsa-miR-583, hsa-miR-501-5p, hsa-miR-199b-5p, hsa-miR-224, hsa-miR-221*, hsa-miR-185, hsa-miR-17*, hsa-miR-378*, hsa-miR-1256, hsa-miR-1321, hsa-miR-523*, hsa-miR-631, hsa-miR-1224-3p, hsa-miR-149, hsa-miR-18a*, hsa-miR-320a, hsa-miR-522, hsa-miR-555, hsa-miR-1228, hsa-miR-1827, hsa-miR-135a*, hsa-miR-663b, hsa-miR-7, hsa-miR-320b, hsa-miR-345, hsa-miR-485-5p, hsa-miR-892a, hsa-miR-33a, hsa-miR-377*, hsa-miR-632, hsa-miR-455-3p, hsa-miR-519b-5p, hsa-miR-519c-5p, hsa-miR-346, hsa-miR-649, hsa-miR-367*, hsa-miR-326, hsa-miR-1183, hsa-miR-433, hsa-miR-1255b, hsa-miR-558, hsa-miR-1225-3p.

Immediate early response 5—IER5—NM_{—}016545—hsa-miR-200b, hsa-miR-642, hsa-miR-513a-5p, hsa-miR-192, hsa-miR-551b*, hsa-miR-483-3p, hsa-miR-30c-1*, hsa-miR-92a-2*, hsa-miR-516a-3p, hsa-miR-486-3p, hsa-miR-302a*, hsa-miR-1277, hsa-miR-216b, hsa-miR-1252, hsa-miR-548p, hsa-miR-374a*, hsa-miR-30c-2*, hsa-miR-1236, hsa-miR-491-5p, hsa-miR-548d-3p, hsa-miR-516b*, hsa-let-7b*, hsa-miR-622, hsa-miR-324-3p, hsa-miR-200c, hsa-miR-147b, hsa-miR-450a, hsa-miR-215, hsa-miR-1291, hsa-miR-605, hsa-miR-384, hsa-miR-429, hsa-miR-593, hsa-miR-629*, hsa-miR-523, hsa-miR-1274a, hsa-miR-149*, hsa-miR-136, hsa-miR-328, hsa-miR-330-5p, hsa-let-7f-1*, hsa-miR-185, hsa-miR-100*, hsa-miR-877*, hsa-miR-339-5p, hsa-miR-3p, hsa-miR-1283, hsa-miR-892a, hsa-miR-488*, hsa-miR-632, hsa-miR-601, hsa-let-7a*, hsa-miR-346, hsa-miR-326.

KIAA0146—KIAA0146—NM_{—}001080394—hsa-miR-200b, hsa-miR-27b*, hsa-miR-625, hsa-miR-548g, hsa-miR-27a, hsa-miR-548m, hsa-miR-302a*, hsa-miR-630, hsa-miR-299-5p, hsa-miR-582-5p, hsa-miR-548o, hsa-miR-1, hsa-miR-200c, hsa-miR-548f, hsa-miR-7-1*, hsa-miR-589*, hsa-miR-518a-5p, hsa-miR-144, hsa-miR-1323, hsa-miR-220b, hsa-miR-429, hsa-miR-27b, hsa-miR-548a-3p, hsa-miR-889, hsa-miR-527, hsa-miR-197, hsa-miR-548c-3p, hsa-miR-548e, hsa-miR-892a, hsa-miR-577, hsa-miR-206, hsa-miR-7-2*, hsa-miR-1183, hsa-miR-1275, hsa-miR-1302, hsa-miR-613.

Transmembrane protein 63A—TMEM63A—NM_{—}014698—hsa-miR-576-3p, hsa-miR-194*, hsa-miR-665, hsa-miR-509-3-5p, hsa-miR-34c-5p, hsa-miR-1254, hsa-miR-650, hsa-miR-1184, hsa-miR-9, hsa-miR-125a-5p, hsa-miR-214, hsa-miR-920, hsa-miR-511, hsa-miR-31*, hsa-miR-486-3p, hsa-miR-449a, hsa-miR-378, hsa-miR-1288, hsa-miR-298, hsa-miR-619, hsa-miR-370, hsa-miR-1207-5p, hsa-miR-1287, hsa-miR-103, hsa-miR-449b, hsa-miR-1236, hsa-miR-618, hsa-miR-324-3p, hsa-miR-1207-3p, hsa-miR-1269, hsa-miR-1202, hsa-miR-765, hsa-miR-296-5p, hsa-miR-599, hsa-miR-502-5p, hsa-miR-591, hsa-miR-1291, hsa-miR-1253, hsa-miR-34a, hsa-miR-193a-3p, hsa-miR-768-5p, hsa-miR-1262, hsa-miR-125b, hsa-miR-596, hsa-miR-940, hsa-miR-324-5p, hsa-miR-588, hsa-miR-432, hsa-miR-1285, hsa-miR-15a*, hsa-miR-612, hsa-miR-629*, hsa-miR-193b, hsa-miR-422a, hsa-miR-1255a, hsa-miR-149*, hsa-miR-328, hsa-miR-492, hsa-miR-361-3p, hsa-miR-1300, hsa-miR-150, hsa-miR-1273, hsa-miR-151-5p, hsa-miR-1321, hsa-miR-100*, hsa-miR-631, hsa-miR-197, hsa-miR-1228, hsa-miR-1827, hsa-miR-656, hsa-miR-1264, hsa-miR-1251, hsa-miR-485-5p, hsa-miR-1278, hsa-miR-331-3p, hsa-miR-488*, hsa-miR-107, hsa-miR-564, hsa-miR-183, hsa-miR-423-5p, hsa-miR-509-5p, hsa-miR-649, hsa-miR-497*, hsa-miR-1255b, hsa-miR-1225-3p, hsa-miR-512-5p.

NEDD4 binding protein 2-like 1—N4BP2L1—Variant 1 and Variant 2—NM_{—}052818 and NM_{—}001079691—hsa-miR-124*, hsa-miR-1254, hsa-miR-448, hsa-miR-22, hsa-miR-219-1-3p, hsa-miR-562, hsa-miR-192, hsa-miR-1276, hsa-miR-520g, hsa-miR-222*, hsa-miR-374b, hsa-miR-551b*, hsa-miR-454*, hsa-miR-195*, hsa-miR-302c*, hsa-miR-668, hsa-miR-548a-5p, hsa-miR-376c, hsa-miR-559, hsa-miR-127-5p, hsa-miR-548b-5p, hsa-miR-452, hsa-miR-16-2*, hsa-miR-320c, hsa-miR-544, hsa-miR-143, hsa-miR-26a-1*, hsa-miR-142-5p, hsa-miR-379*, hsa-miR-29b-1*, hsa-miR-28-5p, hsa-miR-1237, hsa-miR-541*, hsa-miR-548n, hsa-miR-93*, hsa-let-7c*, hsa-miR-548c-5p, hsa-miR-370, hsa-miR-875-3p, hsa-miR-657, hsa-miR-299-5p, hsa-miR-586, hsa-miR-23a, hsa-miR-365, hsa-miR-548i, hsa-miR-520a-5p, hsa-miR-641, hsa-miR-212, hsa-miR-548d-3p, hsa-miR-708, hsa-miR-590-5p, hsa-miR-145*, hsa-miR-525-5p, hsa-miR-582-3p, hsa-miR-30a*, hsa-miR-29a*, hsa-miR-548f, hsa-miR-664, hsa-miR-320d, hsa-miR-590-3p, hsa-miR-29c, hsa-miR-513c, hsa-miR-132, hsa-miR-591, hsa-miR-7-1*, hsa-miR-215, hsa-miR-767-3p, hsa-miR-589*, hsa-miR-29b, hsa-miR-135a, hsa-miR-579, hsa-miR-411*, hsa-miR-26a-2*, hsa-miR-376a*, hsa-miR-186*, hsa-miR-653, hsa-miR-220b, hsa-miR-505*, hsa-miR-1224-5p, hsa-miR-888, hsa-miR-30d*, hsa-miR-802, hsa-miR-30e*, hsa-miR-29a, hsa-miR-570, hsa-miR-517*, hsa-miR-940, hsa-miR-548d-5p, hsa-miR-432, hsa-miR-1270, hsa-miR-135b, hsa-miR-636, hsa-miR-548j, hsa-miR-323-3p, hsa-miR-607, hsa-miR-629*, hsa-miR-29b-2*, hsa-miR-934, hsa-miR-942, hsa-miR-23b, hsa-miR-1322, hsa-miR-1274a, hsa-miR-609, hsa-miR-548a-3p, hsa-miR-620, hsa-miR-302d*, hsa-miR-889, hsa-miR-548h, hsa-miR-381, hsa-miR-150, hsa-miR-342-5p, hsa-miR-1256, hsa-miR-199a-3p, hsa-miR-130a*, hsa-miR-520h, hsa-miR-320a, hsa-miR-522, hsa-miR-1279, hsa-miR-320b, hsa-miR-548e, hsa-miR-21, hsa-miR-199b-3p, hsa-miR-7-2*, hsa-miR-300, hsa-miR-219-5p, hsa-miR-374a, hsa-miR-425, hsa-miR-183, hsa-miR-126*, hsa-miR-338-3p, hsa-miR-767-5p, hsa-miR-218, hsa-miR-302b*, hsa-miR-649, hsa-miR-433, hsa-miR-142-3p, hsa-miR-558, hsa-miR-584.

LY6/PLAUR domain containing 6B—LYPD6B—NM_{—}177964—hsa-miR-376b, hsa-miR-1254, hsa-miR-500, hsa-miR-519b-3p, hsa-miR-377, hsa-miR-92a-2*, hsa-miR-335*, hsa-miR-519c-3p, hsa-miR-892b, hsa-miR-519a, hsa-miR-130b, hsa-miR-301b, hsa-miR-491-5p, hsa-miR-376a, hsa-miR-130a, hsa-miR-548f, hsa-miR-1291, hsa-miR-568, hsa-miR-593, hsa-miR-34b*, hsa-miR-450b-5p, hsa-miR-610, hsa-miR-548a-3p, hsa-miR-412, hsa-miR-301a, hsa-miR-501-5p, hsa-miR-221*, hsa-miR-631, hsa-miR-1827, hsa-miR-454, hsa-miR-548e, hsa-miR-485-5p, hsa-miR-218, hsa-miR-497*, hsa-miR-671-5p.

BEX family member 5—BEX5—NM_{—}001012978—hsa-miR-515-5p, hsa-miR-1290, hsa-miR-548m, hsa-miR-298, hsa-miR-600, hsa-miR-23a, hsa-miR-302f, hsa-miR-32*, hsa-miR-144, hsa-miR-23b, hsa-miR-519e*, hsa-miR-877*, hsa-miR-130a*, hsa-miR-126*.

KIAA1217—KIAA1217—Variant 1, Variant 2 and Variant 3—N_{—}019590, NM_{—}001098500 and NM_{—}001098501—hsa-miR-34c-5p, hsa-miR-130b*, hsa-miR-216a, hsa-miR-19b-2*, hsa-miR-9, hsa-miR-487a, hsa-miR-532-3p, hsa-miR-335, hsa-miR-363*, hsa-miR-15b*, hsa-miR-379*, hsa-miR-335*, hsa-miR-449a, hsa-miR-561, hsa-miR-19b, hsa-miR-943, hsa-miR-194, hsa-miR-548n, hsa-miR-217, hsa-miR-603, hsa-miR-19a*, hsa-miR-342-3p, hsa-miR-409-5p, hsa-miR-130b, hsa-miR-1252, hsa-miR-129*, hsa-miR-148a, hsa-miR-299-5p, hsa-miR-621, hsa-miR-19b-1*, hsa-miR-301b, hsa-miR-449b, hsa-miR-33a*, hsa-miR-606, hsa-miR-641, hsa-let-7b*, hsa-miR-151-3p, hsa-miR-371-5p, hsa-miR-581, hsa-miR-130a, hsa-miR-148b, hsa-miR-624, hsa-miR-635, hsa-miR-1253, hsa-miR-34a, hsa-miR-411*, hsa-miR-873, hsa-miR-518a-5p, hsa-miR-144, hsa-miR-214*, hsa-miR-488, hsa-miR-570, hsa-miR-124, hsa-miR-593, hsa-miR-768-3p, hsa-miR-148b*, hsa-miR-331-5p, hsa-miR-450b-5p, hsa-miR-20a*, hsa-miR-935, hsa-miR-9*, hsa-miR-607, hsa-miR-1260, hsa-miR-152, hsa-miR-301a, hsa-miR-625*, hsa-miR-708*, hsa-miR-593*, hsa-let-7f-1*, hsa-miR-527, hsa-miR-129-3p, hsa-miR-1299, hsa-miR-656, hsa-miR-182, hsa-miR-454, hsa-miR-506, hsa-miR-1251, hsa-miR-892a, hsa-miR-19a, hsa-miR-485-3p, hsa-miR-1244, hsa-miR-126*, hsa-let-7a*, hsa-miR-944, hsa-miR-16-1*, hsa-miR-1183, hsa-miR-142-3p, hsa-miR-135b*, hsa-miR-1225-3p, hsa-miR-154*.

Chromosome 4 open reading frame 7—C4orf7—NM_{—}152997—hsa-miR-1248, hsa-miR-34c-3p, hsa-miR-125a-3p, hsa-miR-578, hsa-miR-138-2*.

Chromosome 9 open reading frame 64—C9orf64—NM_{—}032307—hsa-miR-509-3-5p, hsa-miR-520b, hsa-miR-645, hsa-miR-625, hsa-miR-1184, hsa-miR-520g, hsa-miR-222*, hsa-miR-548g, hsa-let-7g*, hsa-miR-1179, hsa-miR-532-3p, hsa-miR-520a-3p, hsa-miR-142-5p, hsa-miR-330-3p, hsa-miR-1237, hsa-miR-561, hsa-miR-943, hsa-miR-302e, hsa-miR-128, hsa-miR-134, hsa-miR-130b, hsa-miR-580, hsa-miR-548p, hsa-miR-301b, hsa-miR-337-3p, hsa-miR-302c, hsa-miR-548d-3p, hsa-miR-329, hsa-miR-146a*, hsa-miR-371-5p, hsa-miR-548o, hsa-miR-1227, hsa-miR-373, hsa-miR-130a, hsa-miR-548f, hsa-miR-220c, hsa-miR-302d, hsa-miR-624, hsa-miR-188-3p, hsa-miR-96*, hsa-miR-7-1*, hsa-miR-936, hsa-miR-186*, hsa-miR-520c-3p, hsa-miR-498, hsa-miR-1323, hsa-miR-323-5p, hsa-miR-302a, hsa-miR-512-3p, hsa-miR-520d-3p, hsa-miR-519d, hsa-miR-520e, hsa-miR-93, hsa-miR-362-3p, hsa-miR-548a-3p, hsa-miR-301a, hsa-miR-526b*, hsa-miR-744*, hsa-miR-150, hsa-miR-20b, hsa-miR-378*, hsa-miR-640, hsa-miR-106a, hsa-miR-520h, hsa-miR-1228, hsa-miR-17, hsa-miR-106a*, hsa-miR-656, hsa-miR-454, hsa-miR-302b, hsa-miR-548e, hsa-miR-485-5p, hsa-miR-331-3p, hsa-miR-7-2*, hsa-miR-20a, hsa-miR-509-5p, hsa-miR-193b*, hsa-miR-106b, hsa-miR-372, hsa-miR-367*, hsa-miR-497*, hsa-miR-433.

Zyg-11 homolog B—ZYG11B—NM_{—}024646—hsa-miR-576-3p, hsa-miR-140-5p, hsa-miR-376b, hsa-miR-517a, hsa-miR-637, hsa-miR-877, hsa-miR-520b, hsa-miR-34c-5p, hsa-miR-380, hsa-miR-1306, hsa-miR-20b*, hsa-miR-216a, hsa-miR-513a-5p, hsa-miR-766, hsa-miR-125b-2*, hsa-miR-520g, hsa-miR-222*, hsa-miR-101*, hsa-miR-1301, hsa-miR-513b, hsa-miR-195*, hsa-miR-302c*, hsa-miR-548g, hsa-miR-28-3p, hsa-miR-1267, hsa-miR-1248, hsa-miR-335, hsa-miR-127-5p, hsa-miR-452, hsa-miR-16-2*, hsa-miR-575, hsa-miR-520a-3p, hsa-miR-15b*, hsa-miR-31, hsa-miR-513a-3p, hsa-miR-24-1*, hsa-miR-335*, hsa-miR-449a, hsa-miR-548m, hsa-miR-19b, hsa-miR-541*, hsa-miR-548n, hsa-miR-1178, hsa-miR-217, hsa-miR-892b, hsa-miR-302e, hsa-miR-508-5p, hsa-miR-619, hsa-miR-1303, hsa-miR-875-3p, hsa-miR-216b, hsa-miR-1252, hsa-miR-524-5p, hsa-miR-143*, hsa-miR-543, hsa-miR-186, hsa-miR-23a, hsa-miR-449b, hsa-miR-1305, hsa-miR-33a*, hsa-miR-302c, hsa-miR-202, hsa-miR-520a-5p, hsa-miR-24-2*, hsa-miR-548d-3p, hsa-miR-590-5p, hsa-miR-145*, hsa-miR-525-5p, hsa-miR-30a*, hsa-miR-29a*, hsa-miR-622, hsa-miR-376a, hsa-miR-1227, hsa-miR-373, hsa-miR-552, hsa-miR-548f, hsa-miR-484, hsa-miR-32*, hsa-miR-1269, hsa-miR-302d, hsa-miR-590-3p, hsa-miR-495, hsa-miR-513c, hsa-miR-635, hsa-miR-34c-3p, hsa-miR-767-3p, hsa-miR-26b*, hsa-miR-1253, hsa-miR-1200, hsa-miR-34a, hsa-miR-193a-3p, hsa-miR-361-5p, hsa-miR-376a*, hsa-miR-186*, hsa-miR-873, hsa-miR-520c-3p, hsa-miR-518a-5p, hsa-miR-498, hsa-miR-214*, hsa-miR-323-5p, hsa-miR-384, hsa-miR-653, hsa-miR-302a, hsa-miR-888, hsa-miR-30d*, hsa-miR-553, hsa-miR-30e*, hsa-miR-21*, hsa-miR-570, hsa-miR-508-3p, hsa-miR-124, hsa-miR-34b, hsa-miR-1294, hsa-miR-588, hsa-miR-1270, hsa-miR-769-3p, hsa-miR-450b-3p, hsa-miR-512-3p, hsa-miR-520d-3p, hsa-miR-15a*, hsa-miR-331-5p, hsa-miR-450b-5p, hsa-miR-519d, hsa-miR-9*, hsa-miR-664*, hsa-miR-636, hsa-miR-520e, hsa-miR-200a, hsa-miR-10b*, hsa-miR-93, hsa-miR-383, hsa-miR-155*, hsa-miR-193b, hsa-miR-200b*, hsa-miR-491-3p, hsa-miR-1260, hsa-miR-23b, hsa-miR-548a-3p, hsa-miR-634, hsa-miR-149*, hsa-miR-620, hsa-miR-302d*, hsa-miR-141, hsa-miR-889, hsa-miR-526b*, hsa-miR-625*, hsa-miR-744*, hsa-miR-593*, hsa-miR-150, hsa-miR-1273, hsa-miR-181a-2*, hsa-miR-205, hsa-miR-527, hsa-miR-17*, hsa-miR-20b, hsa-miR-378*, hsa-miR-1321, hsa-miR-640, hsa-miR-631, hsa-miR-1224-3p, hsa-miR-106a, hsa-miR-1245, hsa-miR-130a*, hsa-miR-483-5p, hsa-miR-520h, hsa-miR-555, hsa-miR-197, hsa-miR-548c-3p, hsa-miR-17, hsa-miR-506, hsa-miR-302b, hsa-miR-626, hsa-miR-1283, hsa-miR-548e, hsa-miR-1251, hsa-miR-21, hsa-miR-485-5p, hsa-miR-577, hsa-miR-19a, hsa-miR-188-5p, hsa-miR-20a, hsa-miR-409-3p, hsa-miR-455-3p, hsa-miR-126*, hsa-miR-338-3p, hsa-miR-944, hsa-miR-499-5p, hsa-miR-193b*, hsa-miR-140-3p, hsa-miR-106b, hsa-let-7f-2*, hsa-miR-517c, hsa-miR-218, hsa-miR-302b*, hsa-miR-372, hsa-miR-1183, hsa-miR-505, hsa-miR-520d-5p, hsa-miR-200a*, hsa-miR-891b, hsa-miR-1293, hsa-miR-340*, hsa-miR-584.

Family with sequence similarity 160, member B1—FAM160B1—NM_{—}020940—hsa-miR-518d-5p, hsa-miR-200b, hsa-miR-520b, hsa-miR-32, hsa-miR-16, hsa-miR-448, hsa-miR-519a*, hsa-miR-500, hsa-miR-542-3p, hsa-miR-522*, hsa-miR-625, hsa-miR-650, hsa-miR-133a, hsa-miR-1301, hsa-miR-551b*, hsa-miR-302c*, hsa-miR-221, hsa-miR-1179, hsa-miR-532-3p, hsa-miR-27a, hsa-miR-363*, hsa-miR-452, hsa-miR-496, hsa-miR-320c, hsa-miR-520a-3p, hsa-miR-15b*, hsa-miR-513a-3p, hsa-miR-142-5p, hsa-miR-335*, hsa-miR-561, hsa-miR-129-5p, hsa-miR-194, hsa-miR-892b, hsa-miR-146b-3p, hsa-miR-302e, hsa-let-7c*, hsa-miR-92b, hsa-miR-587, hsa-miR-216b, hsa-miR-103, hsa-miR-660, hsa-miR-367, hsa-miR-582-5p, hsa-miR-30b, hsa-miR-25, hsa-miR-374a*, hsa-miR-186, hsa-miR-33a*, hsa-miR-302c, hsa-miR-218-2*, hsa-miR-410, hsa-miR-421, hsa-let-7b*, hsa-miR-151-3p, hsa-miR-582-3p, hsa-miR-411, hsa-miR-30a*, hsa-miR-30a, miR-373, hsa-miR-200c, hsa-miR-145, hsa-miR-874, hsa-miR-548f, hsa-miR-26b, hsa-miR-569, hsa-miR-664, hsa-miR-573, hsa-miR-1269, hsa-miR-302d, hsa-miR-532-5p, hsa-miR-15b, hsa-miR-320d, hsa-miR-590-3p, hsa-miR-495, hsa-miR-1297, hsa-miR-7-1*, hsa-miR-34c-3p, hsa-miR-26b*, hsa-miR-643, hsa-miR-520c-3p, hsa-miR-770-5p, hsa-miR-498, hsa-miR-384, hsa-miR-220b, hsa-miR-505*, hsa-miR-302a, hsa-miR-30d*, hsa-miR-802, hsa-miR-30e*, hsa-miR-429, hsa-miR-570, hsa-miR-508-3p, hsa-miR-940, hsa-miR-34b, hsa-miR-222, hsa-miR-518f*, hsa-miR-768-3p, hsa-miR-148b*, hsa-miR-512-3p, hsa-miR-520d-3p, hsa-miR-195, hsa-miR-450b-5p, hsa-miR-610, hsa-miR-519d, hsa-miR-545, hsa-miR-935, hsa-miR-9*, hsa-miR-27b, hsa-miR-323-3p, hsa-miR-520e, hsa-miR-520c-5p, hsa-miR-200a, hsa-miR-30d, hsa-miR-93, hsa-miR-29b-2*, hsa-miR-155*, hsa-miR-545*, hsa-miR-548a-3p, hsa-miR-133b, hsa-miR-567, hsa-miR-302d*, hsa-miR-141, hsa-miR-223, hsa-miR-889, hsa-miR-526b*, hsa-miR-203, hsa-miR-92a, hsa-miR-381, hsa-miR-597, hsa-miR-361-3p, hsa-miR-518e*, hsa-let-7f-1*, hsa-miR-30c, hsa-miR-526a, hsa-miR-501-5p, hsa-miR-151-5p, hsa-miR-20b, hsa-miR-523*, hsa-miR-1224-3p, hsa-miR-199a-3p, hsa-miR-106a, hsa-miR-1245, hsa-miR-149, hsa-miR-320a, hsa-miR-17, hsa-miR-1279, hsa-miR-656, hsa-miR-320b, hsa-miR-297, hsa-miR-424, hsa-miR-302b, hsa-miR-548e, hsa-miR-199b-3p, hsa-miR-1278, hsa-miR-924, hsa-miR-7-2*, hsa-miR-107, hsa-miR-300, hsa-miR-15a, hsa-miR-1244, hsa-miR-20a, hsa-miR-627, hsa-miR-363, hsa-miR-519b-5p, hsa-let-7a*, hsa-miR-340, hsa-miR-219-2-3p, hsa-miR-106b, hsa-miR-497, hsa-miR-519c-5p, hsa-miR-302b*, hsa-miR-372, hsa-miR-505, hsa-miR-26a, hsa-miR-1250, hsa-miR-512-5p, hsa-miR-30e.

Limb region 1 homolog—LMBR1—NM_{—}022458—hsa-miR-576-3p, hsa-miR-376b, hsa-miR-510, hsa-miR-200b, hsa-miR-509-3-5p, hsa-miR-1266, hsa-miR-448, hsa-miR-380*, hsa-miR-542-3p, hsa-miR-922, hsa-miR-625, hsa-miR-493*, hsa-miR-130b*, hsa-miR-1276, hsa-miR-1184, hsa-miR-1301, hsa-miR-551b*, hsa-miR-454*, hsa-miR-302c*, hsa-miR-548g, hsa-miR-563, hsa-miR-106b*, hsa-miR-490-3p, hsa-miR-127-5p, hsa-miR-320c, hsa-miR-513a-3p, hsa-miR-148a*, hsa-miR-142-5p, hsa-miR-379*, hsa-miR-335*, hsa-miR-183*, hsa-miR-28-5p, hsa-miR-561, hsa-miR-19b, hsa-miR-129-5p, hsa-miR-548n, hsa-miR-217, hsa-miR-892b, hsa-miR-1288, hsa-miR-885-5p, hsa-miR-128, hsa-miR-298, hsa-miR-130b, hsa-miR-455-5p, hsa-miR-875-3p, hsa-miR-1252, hsa-miR-1182, hsa-miR-630, hsa-miR-148a, hsa-miR-548p, hsa-miR-586, hsa-miR-30b, hsa-miR-374a*, hsa-miR-186, hsa-miR-23a, hsa-miR-365, hsa-miR-301b, hsa-miR-33a*, hsa-miR-520a-5p, hsa-miR-641, hsa-miR-151-3p, hsa-miR-708, hsa-miR-146a*, hsa-miR-590-5p, hsa-miR-525-5p, hsa-miR-494, hsa-miR-582-3p, hsa-miR-30a, hsa-miR-548o, hsa-miR-1, hsa-miR-376a, hsa-miR-200c, hsa-miR-130a, hsa-miR-148b, hsa-miR-548f, hsa-miR-99a*, hsa-miR-32*, hsa-miR-532-5p, hsa-miR-320d, hsa-miR-590-3p, hsa-miR-513c, hsa-miR-635, hsa-miR-767-3p, hsa-miR-26b*, hsa-miR-1200, hsa-miR-411*, hsa-miR-361-5p, hsa-miR-873, hsa-miR-27a*, hsa-miR-770-5p, hsa-miR-1262, hsa-miR-1323, hsa-miR-1224-5p, hsa-miR-654-3p, hsa-miR-596, hsa-miR-429, hsa-miR-624*, hsa-miR-570, hsa-miR-508-3p, hsa-miR-34b, hsa-miR-324-5p, hsa-miR-210, hsa-miR-125a-3p, hsa-miR-331-5p, hsa-miR-610, hsa-miR-20a*, hsa-miR-9*, hsa-miR-514, hsa-miR-607, hsa-miR-200a, hsa-miR-30d, hsa-miR-629*, hsa-miR-518c*, hsa-miR-491-3p, hsa-miR-152, hsa-miR-23b, hsa-miR-545*, hsa-miR-539, hsa-miR-548a-3p, hsa-miR-33b, hsa-miR-634, hsa-miR-516b, hsa-miR-33b*, hsa-miR-149*, hsa-miR-302d*, hsa-miR-301a, hsa-miR-141, hsa-miR-22*, hsa-miR-361-3p, hsa-miR-432*, hsa-miR-1300, hsa-miR-30c, hsa-miR-583, hsa-miR-501-5p, hsa-miR-224, hsa-miR-221*, hsa-miR-185, hsa-miR-17*, hsa-miR-1321, hsa-miR-100*, hsa-miR-1245, hsa-miR-130a*, hsa-miR-519e, hsa-miR-550, hsa-miR-320a, hsa-miR-1827, hsa-miR-1299, hsa-miR-548c-3p, hsa-miR-320b, hsa-miR-454, hsa-miR-297, hsa-miR-548k, hsa-miR-99b*, hsa-miR-548l, hsa-miR-548e, hsa-miR-21, hsa-miR-655, hsa-miR-485-5p, hsa-miR-206, hsa-miR-33a, hsa-miR-19a, hsa-miR-485-3p, hsa-miR-601, hsa-miR-183, hsa-miR-423-5p, hsa-miR-1272, hsa-miR-509-5p, hsa-miR-944, hsa-miR-340, hsa-miR-219-2-3p, hsa-miR-515-3p, hsa-miR-140-3p, hsa-miR-7f-2*, hsa-miR-302b*, hsa-miR-649, hsa-miR-1183, hsa-miR-223*, hsa-miR-1250, hsa-miR-1205, hsa-miR-1302, hsa-miR-613, hsa-miR-30e.

Nuclear casein kinase and cyclin-dependent kinase substrate 1—NUCKS1—NM_{—}022731—hsa-miR-576-3p, hsa-miR-140-5p, hsa-miR-124*, hsa-miR-642, hsa-miR-16, hsa-miR-448, hsa-miR-500, hsa-miR-216a, hsa-miR-513a-5p, hsa-miR-1276, hsa-miR-125b-2*, hsa-miR-520g, hsa-miR-150*, hsa-miR-1301, hsa-miR-181a, hsa-miR-551b*, hsa-miR-454*, hsa-miR-519b-3p, hsa-miR-513b, hsa-miR-195*, hsa-miR-302c*, hsa-miR-96, hsa-miR-668, hsa-miR-214, hsa-miR-490-3p, hsa-miR-335, hsa-miR-920, hsa-miR-16-2*, hsa-miR-575, hsa-miR-320c, hsa-miR-511, hsa-miR-15b*, hsa-miR-513a-3p, hsa-miR-142-5p, hsa-miR-379*, hsa-miR-548m, hsa-miR-183*, hsa-miR-330-3p, hsa-miR-190, hsa-miR-302a*, hsa-miR-1237, hsa-miR-561, hsa-miR-519c-3p, hsa-miR-194, hsa-miR-892b, hsa-miR-885-5p, hsa-miR-93*, hsa-miR-603, hsa-miR-1825, hsa-miR-128, hsa-miR-134, hsa-miR-519a, hsa-miR-342-3p, hsa-miR-298, hsa-miR-130b, hsa-miR-1303, hsa-miR-875-3p, hsa-miR-657, hsa-miR-216b, hsa-miR-103, hsa-miR-1252, hsa-miR-129*, hsa-miR-660, hsa-miR-148a, hsa-miR-1246, hsa-miR-299-5p, hsa-miR-600, hsa-miR-548p, hsa-miR-1296, hsa-miR-524-5p, hsa-miR-30b, hsa-miR-143*, hsa-miR-186, hsa-miR-23a, hsa-miR-365, hsa-miR-301b, hsa-miR-1305, hsa-miR-337-3p, hsa-miR-202, hsa-miR-606, hsa-miR-641, hsa-miR-212, hsa-miR-548d-3p, hsa-miR-329, hsa-let-7b*, hsa-miR-151-3p, hsa-miR-146a*, hsa-miR-494, hsa-miR-582-3p, hsa-miR-30a*, hsa-miR-371-5p, hsa-miR-30a, hsa-miR-489, hsa-miR-1, hsa-miR-581, hsa-miR-130a, hsa-miR-145, hsa-miR-148b, hsa-miR-548f, hsa-miR-220c, hsa-miR-1286, hsa-miR-26b, hsa-miR-569, hsa-miR-664, hsa-miR-628-3p, hsa-miR-624, hsa-miR-532-5p, hsa-miR-15b, hsa-miR-320d, hsa-miR-188-3p, hsa-miR-96*, hsa-miR-599, hsa-miR-590-3p, hsa-miR-495, hsa-miR-1271, hsa-miR-29c, hsa-miR-132, hsa-miR-1297, hsa-miR-7-1*, hsa-miR-1208, hsa-miR-34c-3p, hsa-miR-767-3p, hsa-miR-589*, hsa-miR-26b*, hsa-miR-190b, hsa-miR-1200, hsa-miR-29b, hsa-miR-135a, hsa-miR-411*, hsa-miR-361-5p, hsa-miR-146a, hsa-miR-605, hsa-miR-1259, hsa-miR-1233, hsa-miR-770-5p, hsa-miR-498, hsa-miR-214*, hsa-miR-384, hsa-miR-220b, hsa-miR-30d*, hsa-miR-802, hsa-miR-30e*, hsa-miR-29a, hsa-miR-624*, hsa-miR-570, hsa-miR-105, hsa-miR-940, hsa-miR-588, hsa-miR-195, hsa-miR-610, hsa-miR-181d, hsa-miR-135b, hsa-miR-9*, hsa-miR-607, hsa-miR-200a, hsa-miR-30d, hsa-miR-383, hsa-miR-192*, hsa-miR-1206, hsa-miR-934, hsa-miR-554, hsa-miR-152, hsa-miR-23b, hsa-miR-1322, hsa-miR-362-3p, hsa-miR-545*, hsa-miR-539, hsa-miR-1257, hsa-miR-548a-3p, hsa-miR-146b-5p, hsa-miR-412, hsa-miR-516b, hsa-miR-149*, hsa-miR-302d*, hsa-miR-136, hsa-miR-330-5p, hsa-miR-301a, hsa-miR-141, hsa-miR-223, hsa-miR-203, hsa-miR-492, hsa-miR-381, hsa-miR-708*, hsa-let-7f-1*, hsa-miR-30c, hsa-miR-181c, hsa-miR-583, hsa-miR-221*, hsa-miR-378*, hsa-miR-640, hsa-miR-138-2*, hsa-miR-149, hsa-miR-130a*, hsa-miR-758, hsa-miR-520h, hsa-miR-320a, hsa-miR-197, hsa-miR-202*, hsa-miR-1228, hsa-miR-129-3p, hsa-miR-1299, hsa-miR-548c-3p, hsa-miR-196a*, hsa-miR-7, hsa-miR-320b, hsa-miR-454, hsa-miR-548l, hsa-miR-424, hsa-miR-181b, hsa-miR-548e, hsa-miR-655, hsa-miR-485-5p, hsa-miR-137, hsa-miR-892a, hsa-miR-577, hsa-miR-206, hsa-miR-7-2*, hsa-miR-107, hsa-miR-300, hsa-miR-15a, hsa-miR-876-3p, hsa-miR-455-3p, hsa-let-7a*, hsa-miR-340, hsa-miR-499-5p, hsa-miR-193b*, hsa-miR-219-2-3p, hsa-miR-497, hsa-miR-302b*, hsa-miR-367*, hsa-miR-326, hsa-miR-1183, hsa-miR-497*, hsa-miR-433, hsa-miR-505, hsa-miR-154, hsa-miR-499-3p, hsa-miR-26a, hsa-miR-142-3p, hsa-miR-520d-5p, hsa-miR-613, hsa-miR-30e.

Ankyrin repeat domain 13C—ANKRD13C—NM_{—}030816—hsa-miR-576-3p, hsa-miR-194*, hsa-miR-665, hsa-miR-200b, hsa-miR-520b, hsa-miR-32, hsa-miR-1254, hsa-miR-448, hsa-miR-515-5p, hsa-miR-542-3p, hsa-miR-922, hsa-miR-875-5p, hsa-miR-1290, hsa-miR-374b, hsa-miR-1301, hsa-miR-181a, hsa-miR-551b*, hsa-miR-519b-3p, hsa-miR-513b, hsa-miR-182*, hsa-miR-195*, hsa-miR-302c*, hsa-miR-548g, hsa-miR-204, hsa-miR-890, hsa-miR-548a-5p, hsa-miR-559, hsa-miR-1248, hsa-miR-138-1*, hsa-miR-548b-5p, hsa-miR-16-2*, hsa-miR-520a-3p, miR-31, hsa-miR-513a-3p, hsa-miR-148a*, hsa-miR-544, hsa-miR-379*, hsa-miR-335*, hsa-miR-378, hsa-miR-1237, hsa-miR-561, hsa-miR-519c-3p, hsa-miR-19b, hsa-miR-541*, hsa-miR-548n, hsa-miR-217, hsa-miR-1288, hsa-miR-885-5p, hsa-miR-302e, hsa-let-7c*, hsa-miR-548c-5p, hsa-miR-519a, hsa-miR-409-5p, hsa-miR-298, hsa-miR-92b, hsa-miR-130b, hsa-miR-199a-5p, hsa-miR-875-3p, hsa-let-7d*, hsa-miR-1252, hsa-miR-1182, hsa-miR-660, hsa-miR-299-5p, hsa-miR-548p, hsa-miR-586, hsa-miR-367, hsa-miR-338-5p, hsa-miR-524-5p, hsa-miR-543, hsa-miR-25, hsa-miR-301b, hsa-miR-618, hsa-miR-1305, hsa-miR-33a*, hsa-miR-302c, hsa-miR-548i, hsa-miR-218-2*, hsa-miR-641, hsa-miR-29c*, hsa-miR-548d-3p, hsa-let-7b*, hsa-miR-30a*, hsa-miR-371-5p, hsa-miR-373, hsa-miR-200c, hsa-miR-581, hsa-miR-130a, hsa-miR-145, hsa-miR-548f, hsa-miR-569, hsa-miR-99a*, hsa-miR-720, hsa-miR-664, hsa-miR-1269, hsa-miR-302d, hsa-miR-624, hsa-miR-1324, hsa-miR-590-3p, hsa-miR-495, hsa-miR-29c, hsa-miR-7-1*, hsa-miR-589*, hsa-miR-1253, hsa-miR-29b, hsa-miR-579, hsa-miR-411*, hsa-miR-1243, hsa-miR-147, hsa-miR-520c-3p, hsa-miR-27a*, hsa-miR-18b, hsa-miR-302a, hsa-miR-888, hsa-miR-30d*, hsa-miR-802, hsa-miR-30e*, hsa-miR-592, hsa-miR-429, hsa-miR-29a, hsa-miR-488, hsa-miR-570, hsa-miR-124, hsa-miR-548d-5p, hsa-miR-1261, hsa-miR-593, hsa-miR-768-3p, hsa-miR-148b*, hsa-miR-512-3p, hsa-miR-520d-3p, hsa-miR-450b-5p, hsa-miR-519d, hsa-miR-545, hsa-miR-452*, hsa-miR-181d, hsa-miR-548j, hsa-miR-607, hsa-miR-520e, hsa-miR-1258, hsa-miR-200a, hsa-miR-629, hsa-miR-93, hsa-miR-383, hsa-miR-155*, hsa-miR-518c*, hsa-miR-942, hsa-miR-422a, hsa-miR-548a-3p, hsa-miR-526b, hsa-miR-302d*, hsa-miR-301a, hsa-miR-141, hsa-miR-889, hsa-miR-526b*, hsa-miR-203, hsa-miR-548h, hsa-miR-92a, hsa-miR-211, hsa-miR-519e*, hsa-miR-361-3p, hsa-let-7f-1*, hsa-miR-181c, hsa-miR-181a-2*, hsa-miR-501-5p, hsa-miR-199b-5p, hsa-miR-18a, hsa-miR-1284, hsa-miR-659, hsa-miR-20b, hsa-miR-199a-3p, hsa-miR-106a, hsa-miR-1245, hsa-miR-30b*, hsa-miR-548c-3p, hsa-miR-17, hsa-miR-182, hsa-miR-454, hsa-miR-297, hsa-miR-99b*, hsa-miR-5481, hsa-miR-506, hsa-miR-302b, hsa-miR-181b, hsa-miR-1283, hsa-miR-548e, hsa-miR-1251, hsa-miR-655, hsa-miR-199b-3p, hsa-miR-888*, hsa-miR-122, hsa-miR-7-2*, hsa-miR-19a, hsa-miR-219-5p, hsa-miR-374a, hsa-miR-1244, hsa-miR-20a, hsa-miR-183, hsa-miR-363, hsa-let-7a*, hsa-miR-1272, hsa-miR-944, hsa-miR-106b, hsa-miR-296-3p, hsa-miR-302b*, hsa-miR-372, hsa-miR-497*, hsa-miR-223*, hsa-miR-505, hsa-miR-520d-5p, hsa-miR-135b*, hsa-miR-584.

Coiled-coil domain containing 43—CCDC43—Variant 1—NM_{—}144609—hsa-miR-557, hsa-miR-200b, hsa-miR-1266, hsa-miR-515-5p, hsa-miR-542-3p, hsa-miR-625, hsa-miR-216a, hsa-miR-19b-2*, hsa-miR-875-5p, hsa-miR-9, hsa-miR-150*, hsa-miR-374b, hsa-miR-302c*, hsa-miR-548g, hsa-miR-204, hsa-miR-532-3p, hsa-miR-496, hsa-miR-513a-3p, hsa-miR-544, hsa-miR-379*, hsa-miR-507, hsa-miR-548m, hsa-miR-330-3p, hsa-miR-1237, hsa-miR-561, hsa-miR-129-5p, hsa-miR-1178, hsa-miR-19a*, hsa-miR-199a-5p, hsa-miR-216b, hsa-miR-600, hsa-miR-19b-1*, hsa-miR-30b, hsa-miR-186, hsa-miR-302f, hsa-miR-606, hsa-let-7b*, hsa-miR-30a, hsa-miR-200c, hsa-miR-145, hsa-miR-548f, hsa-miR-362-5p, hsa-miR-569, hsa-miR-664, hsa-miR-532-5p, hsa-miR-188-3p, hsa-miR-502-5p, hsa-miR-591, hsa-miR-767-3p, hsa-miR-411*, hsa-miR-374b*, hsa-miR-384, hsa-miR-429, hsa-miR-450b-5p, hsa-miR-30d, hsa-miR-629*, hsa-miR-1206, hsa-miR-539, hsa-miR-548a-3p, hsa-miR-634, hsa-miR-149*, hsa-miR-1185, hsa-miR-211, hsa-miR-519e*, hsa-let-7f-1*, hsa-miR-30c, hsa-miR-199b-5p, hsa-miR-1321, hsa-miR-199a-3p, hsa-miR-138-2*, hsa-miR-877*, hsa-miR-758, hsa-miR-548c-3p, hsa-miR-663b, hsa-miR-106a*, hsa-miR-7, hsa-miR-182, hsa-miR-345, hsa-miR-548e, hsa-miR-199b-3p, hsa-miR-331-3p, hsa-miR-122, hsa-miR-196a, hsa-miR-374a, hsa-miR-1244, hsa-miR-876-3p, hsa-miR-196b, hsa-let-7a*, hsa-miR-767-5p, hsa-miR-944, hsa-miR-340, hsa-miR-10a*, hsa-miR-30e.

CCDC43—Variant 2—NM_{—}001099225—hsa-miR-557, hsa-miR-200b, hsa-miR-1266, hsa-miR-642, hsa-miR-515-5p, hsa-miR-542-3p, hsa-miR-625, hsa-miR-216a, hsa-miR-19b-2*, hsa-miR-875-5p, hsa-miR-1184, hsa-miR-9, hsa-miR-150*, hsa-miR-374b, hsa-miR-1301, hsa-miR-302c*, hsa-miR-548g, hsa-miR-204, hsa-miR-532-3p, hsa-miR-1248, hsa-miR-496, hsa-miR-513a-3p, hsa-miR-544, hsa-miR-379*, hsa-miR-507, hsa-miR-548m, hsa-miR-330-3p, hsa-miR-1237, hsa-miR-561, hsa-miR-129-5p, hsa-miR-1178, hsa-miR-19a*, hsa-miR-199a-5p, hsa-miR-216b, hsa-miR-600, hsa-miR-19b-1*, hsa-miR-30b, hsa-miR-186, hsa-miR-302f, hsa-miR-606, hsa-let-7b*, hsa-miR-30a, hsa-miR-200c, hsa-miR-145, hsa-miR-548f, hsa-miR-362-5p, hsa-miR-569, hsa-miR-664, hsa-miR-532-5p, hsa-miR-188-3p, hsa-miR-502-5p, hsa-miR-591, hsa-miR-767-3p, hsa-miR-411*, hsa-miR-374b*, hsa-miR-384, hsa-miR-429, hsa-miR-450b-5p, hsa-miR-30d, hsa-miR-629*, hsa-miR-1206, hsa-miR-539, hsa-miR-548a-3p, hsa-miR-634, hsa-miR-149*, hsa-miR-1185, hsa-miR-211, hsa-miR-519e*, hsa-let-7f-1*, hsa-miR-30c, hsa-miR-199b-5p, hsa-miR-1321, hsa-miR-199a-3p, hsa-miR-138-2*, hsa-miR-877*, hsa-miR-758, hsa-miR-548c-3p, hsa-miR-663b, hsa-miR-106a*, hsa-miR-7, hsa-miR-182, hsa-miR-345, hsa-miR-548e, hsa-miR-199b-3p, hsa-miR-331-3p, hsa-miR-122, hsa-miR-196a, hsa-miR-374a, hsa-miR-1244, hsa-miR-627, hsa-miR-876-3p, hsa-miR-196b, hsa-let-7a*, hsa-miR-767-5p, hsa-miR-944, hsa-miR-340, hsa-miR-10a*, hsa-miR-154, hsa-miR-30e.

UHRF1 binding protein 1—UHRF1BP1—NM_{—}017754—hsa-miR-576-3p, hsa-miR-194*, hsa-miR-124*, hsa-miR-557, hsa-miR-510, hsa-miR-200b, hsa-miR-520b, hsa-miR-1254, hsa-miR-642, hsa-miR-16, hsa-miR-448, hsa-miR-515-5p, hsa-miR-22, hsa-miR-625, hsa-miR-138, hsa-miR-219-1-3p, hsa-miR-562, hsa-miR-875-5p, hsa-miR-513a-5p, hsa-miR-766, hsa-miR-1276, hsa-miR-9, hsa-miR-125b-2*, hsa-miR-520g, hsa-miR-1290, hsa-miR-454*, hsa-miR-195*, hsa-miR-483-3p, hsa-miR-204, hsa-miR-96, hsa-miR-28-3p, hsa-miR-532-3p, hsa-miR-377, hsa-miR-218-1*, hsa-miR-1304, hsa-miR-548a-5p, hsa-miR-490-3p, hsa-miR-559, hsa-miR-1267, hsa-miR-1248, hsa-miR-335, hsa-miR-127-5p, hsa-miR-548b-5p, hsa-miR-16-2*, hsa-miR-575, hsa-miR-496, hsa-miR-320c, hsa-miR-520a-3p, hsa-miR-31, hsa-miR-513a-3p, hsa-miR-142-5p, hsa-miR-379*, hsa-miR-507, hsa-miR-29b-1*, hsa-miR-548m, hsa-miR-378, hsa-miR-190, hsa-miR-302a*, hsa-miR-1237, hsa-miR-561, hsa-miR-541*, hsa-miR-302e, hsa-miR-769-5p, hsa-miR-548c-5p, hsa-miR-603, hsa-miR-128, hsa-miR-508-5p, hsa-miR-130b, hsa-miR-455-5p, hsa-miR-619, hsa-miR-587, hsa-miR-1303, hsa-miR-1207-5p, hsa-miR-875-3p, hsa-miR-1252, hsa-miR-630, hsa-miR-129*, hsa-miR-576-5p, hsa-miR-582-5p, hsa-miR-30b, hsa-miR-143*, hsa-miR-543, hsa-miR-374a*, hsa-miR-23a, hsa-miR-301b, hsa-miR-618, hsa-miR-302c, hsa-miR-302f, hsa-miR-548i, hsa-miR-202, hsa-miR-641, hsa-miR-329, hsa-miR-146a*, hsa-miR-145*, hsa-miR-582-3p, hsa-miR-652, hsa-miR-30a, hsa-miR-29a*, hsa-miR-324-3p, hsa-miR-1227, hsa-miR-373, hsa-miR-200c, hsa-miR-581, hsa-miR-1207-3p, hsa-miR-130a, hsa-miR-362-5p, hsa-miR-220c, hsa-miR-1286, hsa-miR-26b, hsa-miR-573, hsa-miR-302d, hsa-miR-15b, hsa-miR-320d, hsa-miR-96*, hsa-miR-590-3p, hsa-miR-502-5p, hsa-miR-1271, hsa-miR-1297, hsa-miR-7-1*, hsa-miR-939, hsa-miR-34c-3p, hsa-miR-26b*, hsa-miR-1253, hsa-miR-190b, hsa-miR-1200, hsa-miR-643, hsa-miR-135a, hsa-miR-411*, hsa-miR-520f, hsa-miR-146a, hsa-miR-605, hsa-miR-1243, hsa-miR-374b*, hsa-miR-186*, hsa-miR-1233, hsa-miR-873, hsa-miR-520c-3p, hsa-miR-608, hsa-miR-770-5p, hsa-miR-509-3p, hsa-miR-498, hsa-miR-1262, hsa-miR-653, hsa-miR-220b, hsa-miR-505*, hsa-miR-302a, hsa-miR-1224-5p, hsa-miR-888, hsa-miR-802, hsa-miR-568, hsa-miR-21*, hsa-miR-429, hsa-miR-488, hsa-miR-570, hsa-miR-548d-5p, hsa-miR-325, hsa-miR-324-5p, hsa-miR-588, hsa-miR-768-3p, hsa-miR-125a-3p, hsa-miR-512-3p, hsa-miR-520d-3p, hsa-miR-195, hsa-miR-519d, hsa-miR-935, hsa-miR-135b, hsa-miR-548j, hsa-miR-520e, hsa-miR-578, hsa-miR-30d, hsa-miR-10b*, hsa-miR-93, hsa-miR-554, hsa-miR-200b*, hsa-miR-518c*, hsa-miR-1260, hsa-miR-422a, hsa-miR-23b, hsa-miR-1322, hsa-miR-362-3p, hsa-miR-1257, hsa-miR-1255a, hsa-miR-146b-5p, hsa-miR-526b, hsa-miR-136, hsa-miR-328, hsa-miR-330-5p, hsa-miR-139-5p, hsa-miR-301a, hsa-miR-223, hsa-miR-889, hsa-miR-526b*, hsa-miR-203, hsa-miR-548h, hsa-miR-708*, hsa-miR-211, hsa-miR-519e*, hsa-miR-1300, hsa-miR-150, hsa-miR-30c, hsa-miR-181a-2*, hsa-miR-760, hsa-miR-583, hsa-miR-224, hsa-miR-151-5p, hsa-miR-221*, hsa-miR-659, hsa-miR-185, hsa-miR-20b, hsa-miR-556-5p, hsa-miR-1321, hsa-miR-640, hsa-miR-1224-3p, hsa-miR-106a, hsa-miR-1245, hsa-miR-138-2*, hsa-miR-877*, hsa-miR-130a*, hsa-miR-758, hsa-miR-520h, hsa-miR-320a, hsa-miR-1225-5p, hsa-miR-1228, hsa-miR-129-3p, hsa-miR-24, hsa-miR-548c-3p, hsa-miR-135a*, hsa-miR-17, hsa-miR-196a*, hsa-miR-106a*, hsa-miR-382, hsa-miR-656, hsa-miR-320b, hsa-miR-454, hsa-miR-1264, hsa-miR-297, hsa-miR-548l, hsa-miR-424, hsa-miR-302b, hsa-miR-655, hsa-miR-137, hsa-miR-331-3p, hsa-miR-888*, hsa-miR-7-2*, hsa-miR-188-5p, hsa-miR-15a, hsa-miR-1244, hsa-miR-566-3p, hsa-miR-20a, hsa-miR-425, hsa-miR-876-3p, hsa-miR-455-3p, hsa-miR-1272, hsa-miR-944, hsa-miR-340, hsa-miR-499-5p, hsa-miR-140-3p, hsa-miR-106b, hsa-miR-497, hsa-miR-369-3p, hsa-miR-296-3p, hsa-let-7f-2*, hsa-miR-372, hsa-miR-326, hsa-miR-497*, hsa-miR-154, hsa-miR-499-3p, hsa-miR-602, hsa-miR-26a, hsa-miR-1275, hsa-miR-1255b, hsa-miR-200a*, hsa-miR-1302, hsa-miR-1293, hsa-miR-512-5p, hsa-miR-584, hsa-miR-30e.

Chromosome 1 open reading frame 163—C1orf163—NM_{—}023077—hsa-miR-576-3p, hsa-miR-34c-5p, hsa-miR-642, hsa-miR-380*, hsa-miR-22, hsa-miR-513a-5p, hsa-miR-766, hsa-let-7g*, hsa-miR-563, hsa-miR-214, hsa-miR-363*, hsa-miR-127-5p, hsa-miR-661, hsa-miR-449a, hsa-miR-28-5p, hsa-miR-129-5p, hsa-miR-943, hsa-miR-508-5p, hsa-miR-657, hsa-miR-1252, hsa-miR-449b, hsa-miR-708, hsa-miR-145*, hsa-miR-29a*, hsa-miR-616*, hsa-miR-373*, hsa-miR-220c, hsa-miR-1202, hsa-miR-502-5p, hsa-miR-589*, hsa-miR-26b*, hsa-miR-34a, hsa-miR-498, hsa-miR-185*, hsa-miR-888, hsa-miR-592, hsa-miR-192*, hsa-miR-567, hsa-miR-149*, hsa-miR-597, hsa-miR-744*, hsa-miR-1321, hsa-miR-30b*, hsa-miR-758, hsa-miR-24, hsa-miR-297, hsa-miR-1283, hsa-miR-187*, hsa-miR-488*, hsa-miR-577, hsa-miR-1272, hsa-miR-558, hsa-miR-1293.

Axin interactor, dorsalization associated—AIDA—NM_{—}022831—hsa-miR-576-3p, hsa-miR-32, hsa-miR-448, hsa-miR-27b*, hsa-miR-922, hsa-miR-19b-2*, hsa-miR-1276, hsa-miR-150*, hsa-miR-1290, hsa-miR-519b-3p, hsa-miR-302c*, hsa-miR-1289, hsa-miR-28-3p, hsa-miR-549, hsa-miR-218-1*, hsa-miR-548a-5p, hsa-miR-559, hsa-miR-27a, hsa-miR-1267, hsa-miR-1248, hsa-miR-548b-5p, hsa-miR-516a-3p, hsa-miR-15b*, hsa-miR-24-1*, hsa-miR-335*, hsa-miR-548m, hsa-miR-378, hsa-miR-330-3p, hsa-miR-561, hsa-miR-519c-3p, hsa-miR-548n, hsa-miR-217, hsa-miR-548c-5p, hsa-miR-19a*, hsa-miR-519a, hsa-miR-92b, hsa-miR-130b, hsa-miR-1280, hsa-miR-875-3p, hsa-miR-657, hsa-miR-580, hsa-miR-600, hsa-miR-548p, hsa-miR-586, hsa-miR-367, hsa-miR-19b-1*, hsa-miR-30b, hsa-miR-25, hsa-miR-301b, hsa-miR-618, hsa-miR-337-3p, hsa-miR-548i, hsa-miR-218-2*, hsa-miR-202, hsa-miR-24-2*, hsa-miR-641, hsa-miR-516b*, hsa-miR-146a*, hsa-miR-582-3p, hsa-miR-30a, hsa-miR-130a, hsa-miR-220c, hsa-miR-664, hsa-miR-484, hsa-miR-573, hsa-miR-32*, hsa-miR-1269, hsa-miR-628-3p, hsa-miR-1324, hsa-miR-96*, hsa-miR-361-5p, hsa-miR-186*, hsa-miR-144, hsa-miR-653, hsa-miR-802, hsa-miR-488, hsa-miR-124, hsa-miR-548d-5p, hsa-miR-1285, hsa-miR-512-3p, hsa-miR-20a*, hsa-miR-452*, hsa-miR-612, hsa-miR-514, hsa-miR-27b, hsa-miR-299-3p, hsa-miR-548j, hsa-miR-30d, hsa-miR-29b-2*, hsa-miR-422a, hsa-miR-33b, hsa-miR-301a, hsa-miR-548h, hsa-miR-92a, hsa-miR-30c, hsa-miR-17*, hsa-miR-100*, hsa-miR-1225-5p, hsa-miR-1299, hsa-miR-1279, hsa-miR-454, hsa-miR-345, hsa-miR-506, hsa-miR-187*, hsa-miR-1251, hsa-miR-655, hsa-miR-137, hsa-miR-888*, hsa-miR-33a, hsa-miR-425, hsa-miR-409-3p, hsa-miR-363, hsa-miR-944, hsa-miR-369-3p, hsa-miR-16-1*, hsa-miR-649, hsa-miR-584, hsa-miR-30e.

Chromosome 4 open reading frame 32—C4orf32—NM_{—}152400—hsa-miR-645, hsa-miR-493*, hsa-miR-648, hsa-miR-216a, hsa-miR-195*, hsa-let-7g*, hsa-miR-548a-5p, hsa-miR-490-3p, hsa-miR-559, hsa-miR-27a, hsa-miR-920, hsa-miR-92a-2*, hsa-miR-548b-5p, hsa-miR-16-2*, hsa-miR-513a-3p, hsa-miR-143, hsa-miR-335*, hsa-miR-183*, hsa-miR-561, hsa-miR-548n, hsa-miR-885-5p, hsa-miR-548c-5p, hsa-miR-199a-5p, hsa-miR-424*, hsa-miR-651, hsa-miR-576-5p, hsa-miR-548p, hsa-miR-338-5p, hsa-miR-524-5p, hsa-miR-143*, hsa-miR-186, hsa-miR-1305, hsa-miR-10a, hsa-miR-33a*, hsa-miR-491-5p, hsa-miR-548i, hsa-miR-410, hsa-miR-421, hsa-let-7b*, hsa-miR-489, hsa-miR-29a*, hsa-miR-324-3p, hsa-miR-548o, hsa-miR-573, hsa-miR-590-3p, hsa-miR-29c, hsa-miR-34c-3p, hsa-miR-1253, hsa-miR-29b, hsa-miR-518a-5p, hsa-miR-144, hsa-miR-1323, hsa-miR-568, hsa-miR-21*, hsa-miR-29a, hsa-miR-570, hsa-miR-105, hsa-miR-548d-5p, hsa-miR-487b, hsa-miR-144*, hsa-miR-20a*, hsa-miR-545, hsa-miR-504, hsa-miR-27b, hsa-miR-548j, hsa-miR-607, hsa-miR-101, hsa-miR-1274a, hsa-miR-545*, hsa-miR-136, hsa-miR-203, hsa-miR-548h, hsa-miR-381, hsa-miR-744*, hsa-let-7f-1*, hsa-miR-1300, hsa-miR-199b-5p, hsa-miR-527, hsa-miR-199a-3p, hsa-miR-10b, hsa-miR-548c-3p, hsa-miR-656, hsa-miR-1264, hsa-miR-1274b, hsa-miR-199b-3p, hsa-miR-137, hsa-miR-1278, hsa-miR-888*, hsa-miR-122, hsa-miR-196a, hsa-miR-300, hsa-miR-601, hsa-miR-196b, hsa-let-7a*, hsa-miR-574-5p, hsa-miR-340, hsa-miR-142-3p, hsa-miR-520d-5p, hsa-miR-891b.

Nestin—NES—NM_{—}006617—hsa-miR-665, hsa-miR-509-3-5p, hsa-miR-138, hsa-miR-650, hsa-miR-204, hsa-miR-31, hsa-miR-658, hsa-miR-1825, hsa-miR-1268, hsa-miR-1207-5p, hsa-miR-635, hsa-miR-1200, hsa-miR-768-5p, hsa-miR-940, hsa-miR-1294, hsa-miR-432, hsa-miR-512-3p, hsa-miR-155*, hsa-miR-136, hsa-miR-330-5p, hsa-miR-211, hsa-miR-17*, hsa-miR-30b*, hsa-miR-338-3p, hsa-miR-509-5p, hsa-miR-371-3p, hsa-miR-326, hsa-miR-154.

v-kit Hardy Zuckerman 4 feline sarcoma viral oncogene homolog—KIT—Variant 1 and Variant 2—NM_{—}000222 and NM_{—}001093772—hsa-miR-194*, hsa-miR-124*, hsa-miR-510, hsa-miR-520b, hsa-miR-1266, hsa-miR-34c-5p, hsa-miR-642, hsa-miR-515-5p, hsa-miR-625, hsa-miR-493*, hsa-miR-648, hsa-miR-875-5p, hsa-miR-519b-3p, hsa-miR-195*, hsa-miR-487a, hsa-miR-221, hsa-miR-483-3p, hsa-miR-548g, hsa-miR-28-3p, hsa-miR-335, hsa-miR-138-1*, hsa-miR-127-5p, hsa-miR-452, hsa-miR-16-2*, hsa-miR-520a-3p, hsa-miR-142-5p, hsa-miR-335*, hsa-miR-449a, hsa-miR-519c-3p, hsa-miR-19b, hsa-miR-302e, hsa-miR-1825, hsa-miR-128, hsa-miR-519a, hsa-miR-508-5p, hsa-miR-130b, hsa-miR-216b, hsa-miR-148a, hsa-miR-576-5p, hsa-miR-582-5p, hsa-miR-301b, hsa-miR-449b, hsa-miR-618, hsa-miR-33a*, hsa-miR-302c, hsa-miR-218-2*, hsa-miR-641, hsa-miR-548d-3p, hsa-let-7b*, hsa-miR-494, hsa-miR-30a*, hsa-miR-548o, hsa-miR-373, hsa-miR-130a, hsa-miR-148b, hsa-miR-573, hsa-miR-302d, hsa-miR-532-5p, hsa-miR-34a, hsa-miR-643, hsa-miR-193a-3p, hsa-miR-520c-3p, hsa-miR-518a-5p, hsa-miR-18b, hsa-miR-1262, hsa-miR-1323, hsa-miR-505*, hsa-miR-302a, hsa-miR-30d*, hsa-miR-30e*, hsa-miR-21*, hsa-miR-517*, hsa-miR-222, hsa-miR-136*, hsa-miR-125a-3p, hsa-miR-512-3p, hsa-miR-520d-3p, hsa-miR-450b-5p, hsa-miR-664*, hsa-miR-520e, hsa-miR-200a, hsa-miR-193b, hsa-miR-1206, hsa-miR-942, hsa-miR-152, hsa-miR-33b*, hsa-miR-139-5p, hsa-miR-301a, hsa-miR-141, hsa-miR-889, hsa-miR-571, hsa-miR-381, hsa-miR-519e*, hsa-miR-361-3p, hsa-let-7f-1*, hsa-miR-432*, hsa-miR-583, hsa-miR-501-5p, hsa-miR-342-5p, hsa-miR-493, hsa-miR-18a, hsa-miR-1284, hsa-miR-527, hsa-miR-1321, hsa-miR-519e, hsa-miR-522, hsa-miR-548c-3p, hsa-miR-135a*, hsa-miR-196a*, hsa-miR-454, hsa-miR-548l, hsa-miR-302b, hsa-miR-137, hsa-miR-488*, hsa-miR-19a, hsa-miR-300, hsa-miR-627, hsa-miR-455-3p, hsa-let-7a*, hsa-miR-338-3p, hsa-miR-515-3p, hsa-miR-296-3p, hsa-let-7f-2*, hsa-miR-218, hsa-miR-372, hsa-miR-505, hsa-miR-595, hsa-miR-154*, hsa-miR-671-5p.

Myeloid cell leukemia sequence 1—MCL1—Variant 1 and Variant 2—NM_{—}021960 and NM_{—}182763—hsa-miR-124*, hsa-miR-510, hsa-miR-877, hsa-miR-520b, hsa-miR-32, hsa-miR-645, hsa-miR-448, hsa-miR-493*, hsa-miR-130b*, hsa-miR-133a, hsa-miR-222*, hsa-miR-101*, hsa-miR-125a-5p, hsa-miR-204, hsa-let-7g*, hsa-miR-1289, hsa-miR-668, hsa-miR-214, hsa-miR-532-3p, hsa-miR-890, hsa-miR-548a-5p, hsa-miR-559, hsa-miR-27a, hsa-miR-92a-2*, hsa-miR-127-5p, hsa-miR-548b-5p, hsa-miR-575, hsa-miR-496, hsa-miR-320c, hsa-miR-520a-3p, hsa-miR-15b*, hsa-miR-513a-3p, hsa-miR-142-5p, hsa-miR-29b-1*, hsa-miR-561, hsa-miR-943, hsa-miR-548n, hsa-miR-217, hsa-miR-1288, hsa-miR-885-5p, hsa-miR-302e, hsa-miR-548c-5p, hsa-miR-603, hsa-miR-128, hsa-miR-92b, hsa-miR-1303, hsa-miR-1207-5p, hsa-miR-623, hsa-miR-339-3p, hsa-miR-1252, hsa-miR-600, hsa-miR-367, hsa-miR-582-5p, hsa-miR-524-5p, hsa-miR-34a*, hsa-miR-25, hsa-miR-374a*, hsa-miR-186, hsa-miR-1236, hsa-miR-618, hsa-miR-337-3p, hsa-miR-302c, hsa-miR-491-5p, hsa-miR-548i, hsa-miR-548d-3p, hsa-let-7b*, hsa-miR-146a*, hsa-miR-153, hsa-miR-582-3p, hsa-miR-622, hsa-miR-324-3p, hsa-miR-373, hsa-miR-362-5p, hsa-miR-26b, hsa-miR-573, hsa-miR-302d, hsa-miR-624, hsa-miR-296-5p, hsa-miR-1324, hsa-miR-320d, hsa-miR-617, hsa-miR-495, hsa-miR-29c, hsa-miR-1297, hsa-miR-939, hsa-miR-589*, hsa-miR-26b*, hsa-miR-1200, hsa-miR-29b, hsa-miR-193a-3p, hsa-miR-361-5p, hsa-miR-520f, hsa-miR-768-5p, hsa-miR-1243, hsa-miR-186*, hsa-miR-520c-3p, hsa-miR-518a-5p, hsa-miR-125b, hsa-miR-302a, hsa-miR-888, hsa-miR-596, hsa-miR-29a, hsa-miR-488, hsa-miR-517*, hsa-miR-940, hsa-miR-548d-5p, hsa-miR-588, hsa-miR-432, hsa-miR-520d-3p, hsa-miR-519d, hsa-miR-935, hsa-miR-514, hsa-miR-27b, hsa-miR-664*, hsa-miR-548j, hsa-miR-607, hsa-miR-520e, hsa-miR-578, hsa-miR-93, hsa-miR-192*, hsa-miR-193b, hsa-miR-1260, hsa-miR-942, hsa-miR-101, hsa-miR-545*, hsa-miR-133b, hsa-miR-149*, hsa-miR-139-5p, hsa-miR-526b*, hsa-miR-22*, hsa-miR-548h, hsa-miR-92a, hsa-miR-597, hsa-miR-1185, hsa-miR-211, hsa-let-7f-1*, hsa-miR-760, hsa-miR-1284, hsa-miR-221*, hsa-miR-527, hsa-miR-20b, hsa-miR-1321, hsa-miR-1224-3p, hsa-miR-106a, hsa-miR-320a, hsa-miR-17, hsa-miR-320b, hsa-miR-1264, hsa-miR-548k, hsa-miR-548l, hsa-miR-302b, hsa-miR-626, hsa-miR-1251, hsa-miR-1278, hsa-miR-556-3p, hsa-miR-20a, hsa-miR-876-3p, hsa-miR-455-3p, hsa-miR-363, hsa-let-7a*, hsa-miR-767-5p, hsa-miR-944, hsa-miR-193b*, hsa-miR-106b, hsa-miR-372, hsa-miR-10a*, hsa-miR-497*, hsa-miR-26a, hsa-miR-520d-5p.

GLI-Kruppel family member GLI2—GLI2—NM_{—}005270—hsa-miR-124*, hsa-miR-376b, hsa-miR-502-3p, hsa-miR-637, hsa-miR-877, hsa-miR-448, hsa-miR-138, hsa-miR-19b-2*, hsa-miR-766, hsa-miR-221, hsa-miR-483-3p, hsa-miR-193a-5p, hsa-miR-1179, hsa-miR-923, hsa-miR-890, hsa-miR-548a-5p, hsa-miR-490-3p, hsa-miR-559, hsa-miR-27a, hsa-miR-92a-2*, hsa-miR-363*, hsa-miR-548b-5p, hsa-miR-575, hsa-miR-15b*, hsa-miR-544, hsa-miR-335*, hsa-let-7c*, hsa-miR-769-5p, hsa-miR-548c-5p, hsa-miR-19a*, hsa-miR-298, hsa-miR-619, hsa-miR-1303, hsa-miR-875-3p, hsa-miR-1252, hsa-miR-1203, hsa-miR-19b-1*, hsa-miR-30b, hsa-miR-1236, hsa-miR-491-5p, hsa-miR-548i, hsa-miR-202, hsa-miR-371-5p, hsa-miR-30a, hsa-miR-376a, hsa-miR-1207-3p, hsa-let-7i*, hsa-miR-664, hsa-miR-484, hsa-miR-624, hsa-miR-296-5p, hsa-miR-29c, hsa-miR-1291, hsa-miR-1200, hsa-miR-885-3p, hsa-miR-29b, hsa-miR-579, hsa-miR-220b, hsa-miR-29a, hsa-miR-488, hsa-miR-105, hsa-miR-548d-5p, hsa-miR-222, hsa-miR-593, hsa-miR-1285, hsa-miR-512-3p, hsa-miR-452*, hsa-miR-612, hsa-miR-27b, hsa-miR-548j, hsa-miR-200a, hsa-miR-30d, hsa-miR-10b*, hsa-miR-518c*, hsa-miR-942, hsa-miR-33b, hsa-miR-516b, hsa-miR-567, hsa-miR-330-5p, hsa-miR-139-5p, hsa-miR-141, hsa-miR-548h, hsa-miR-1185, hsa-miR-432*, hsa-miR-30c, hsa-miR-760, hsa-miR-501-5p, hsa-miR-877*, hsa-miR-202*, hsa-miR-182, hsa-miR-1264, hsa-miR-485-5p, hsa-miR-892a, hsa-miR-33a, hsa-miR-455-3p, hsa-miR-501-3p, hsa-miR-218, hsa-miR-326, hsa-miR-1275, hsa-miR-891b, hsa-miR-512-5p, hsa-miR-30e.

Interleukin 4—IL4—Variant 1 and Variant 2—NM_{—}000589 and NM_{—}172348—hsa-miR-9*, hsa-miR-1272.

The miRNAs predicted to target CSC-related genes are particularly useful targets for therapeutic intervention in prostate and other cancers through manipulation of their expression levels.

### Example 4

### MiRNAs Differentially Expressed in CD44⁺ Cancer Stem Cell from Freshly Isolated Human Prostate Tumor Samples

The CD44-expressing subset in human prostate xenografts is highly enriched in cells that have greater tumorigenic and metastatic potential. CD44⁺ cells also preferentially express self-renewal genes such as Smoothened, Oct-3/4, Bmi, and β-catenin that are aberrantly regulated in a variety of cancers (Patrawala et al., 2006). In acute myeloid leukemia (AML) and liver cancer, targeting the CD44⁺ cancer stem cells effectively eradicates the cancer cells highlighting the functional importance of this population in driving tumor growth (Jin et al., 2006; Xie et al., 2007).

We have shown that CD44⁺ cells obtained from LAPC4, LAPC9, and Du145 prostate cancer xenografts differentially express miRNAs compared with CD44⁻ cells (Table 1). To investigate the expression of some of the differentially regulated miRNAs in clinical specimen, we obtained sixteen, freshly isolated, human prostate tumor samples and immediately dissociated them as follows: tumor tissue was minced in RPMI+10% FBS and incubated 12-14 hours in 200 U/ml Collagenase I solution (Cat. No. C-0310, Sigma-Aldrich, St. Louis, Mo.) to degrade the extracellular matrix. To break up the organoids, the sample was incubated with 2.5% trypsin (cat. no. 17-160E; Biowhittaker Inc., Walkersville, Md., USA) for 15 minutes. The dissociated cells were then passed through an 80 μM nylon mesh to obtain a single cell suspension. Contaminating hematopoietic and stromal cells were removed using the MACS Lineage Cell Depletion kit (Cat. No. 130-092-211; Miltenyi Biotec, Inc.; Auburn, Calif., USA). The cell suspension was stained for 15 minutes at 4° C. first with a fluorescein isothiocyanate (FITC)-conjugated antibody against CD44 (BD Biosciences, San Jose, Calif., USA) and next with anti-FITC microbeads (Cat. No. 130-048-701; Miltenyi Biotec, Inc). The cells were sorted by passing through a MACS column (Cat. No. 130-042-201; Miltenyi Biotec, Inc.), which binds to CD44⁺ cells leaving the CD44⁻ cells in the effluent. The purity of the CD44⁺ fraction thus obtained ranged from 60-95%.

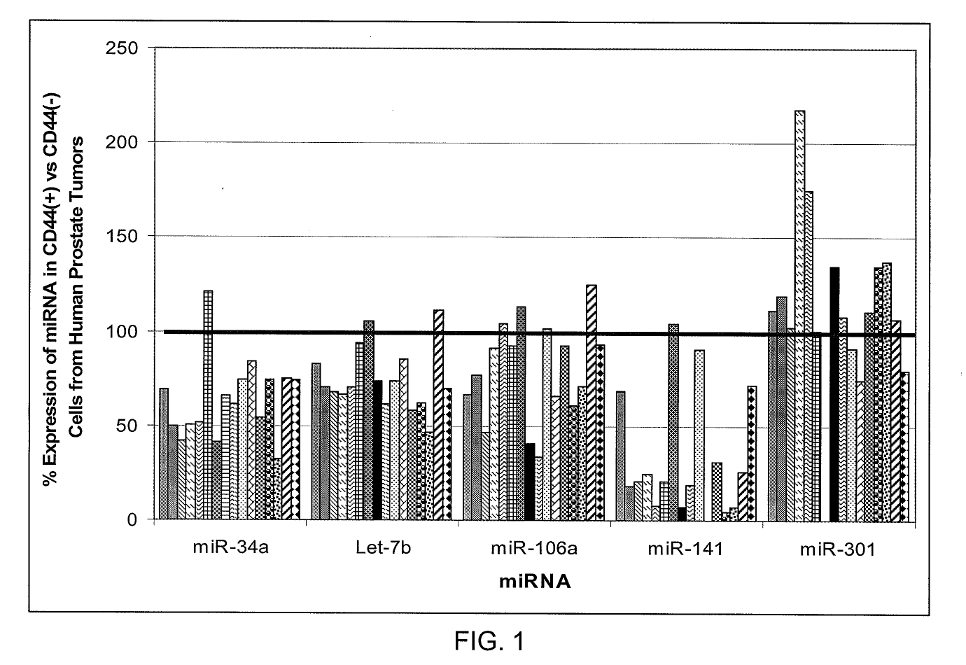

Total RNA was extracted using the mirVana™ miRNA Isolation kit (cat. no. AM1561; Ambion, Inc.; Austin, Tex., USA) according to the manufacturer's recommended protocol. Expression of miR-34a, let-7b, miR-106a, miR-141, miR-301, miR-24, and miR-103 was determined in duplicate by quantitative real-time PCR using (1) RNA from cells derived from prostate cancer cells carrying the CD44 epitope (CD44⁺) and (2) RNA from the population of tumor cells recovered after CSC removal from the respective tumors. Quantitative real-time PCR assays employed TaqMan® MicroRNA Assay reagents (Applied Biosystems, Foster City, Calif., USA). Raw Ct values from CSCs were normalized to those of the housekeeping miRNAs miR-24, miR-26a, and miR-191 and expressed relative to the normalized values generated in the CSC-depleted prostate tumor cell population (FIG. 1). All five of the therapeutic miRNAs tested showed the same expression trend as in the CD44⁺ CSCs obtained from xenografts (FIG. 1 and Table 1). Four of the miRNAs, hsa-miR-34a, let-7b, miR-106a, and hsa-miR-141, were down-regulated overall. miR-301 was upregulated in CSCs from patient samples as well as from xenografts.

miR-141, let-7b, miR-106a, and miR-34a are downregulated in CSCs derived from clinical prostate tumor specimens and in CSCs purified from prostate cancer xenografts, suggesting that they have an anti-oncogenic function. Replacement of these miRNAs can provide a potential therapeutic benefit in prostate cancer and in other cancers. Overexpression of miR-301 in CSCs indicates an oncogenic role, making it a potential target for cancer therapy using a miRNA antagonist.

### Example 5

### HSA-MIR-34A Inhibits Cancer Stem Cell Growth in Vitro and in Vivo

Anchorage-independent growth, also known as clonogenicity, is a hallmark of stem cells and CSCs from solid tumors. In brain and breast tissue, neurosphere and mammosphere assays are used to enrich for normal stem cells as well as cancer stem cells, as most differentiated cells perish in non-adherent culture conditions (Reynolds et al., 1992; Singh et al., 2003; Dontu et al., 2003; Liu et al., 2006). Prostaspheres are spherical groups of cells generated from anchorage-independent growth of prostate cancer cells that maintain higher levels of CD133 expression and that are capable of undergoing differentiation with an appropriate stimulus (Miki et al., 2007). CD44⁺ cells from LAPC4 and LAPC9 prostate xenografts are 10-100 fold enriched in sphere-forming capacity, and comprise a spectrum of differentiated and undifferentiated cells indicating that the cell of origin is a CSC (Patrawala et al., 2006). Clonogenic assays, therefore, are an important tool for measuring the frequency of CSCs in a bulk population.

Hsa-miR-34a was downregulated in all the CSC fractions analyzed (Tables 1 and 2 above), suggesting that this miRNA may play a critical role in antagonizing CSC properties. The inventors therefore investigated whether re-expressing miR-34a in prostate cancer cells would have a deleterious effect on the CSC population as measured by efficiency of sphere formation (EOS). LAPC4 cells (1.25×10³) were transfected with 5 μg (1.6 μM) miR-34a (Pre-miR™-hsa-miR-34a, cat. no. 17100; Ambion) or a sequence-scrambled negative control miRNA (Pre-miR™ MicroRNA Precursor Molecule-Negative Control #2, cat. no. 17111; Ambion) by electroporation and seeded in triplicate into 60% Matrigel (Becton, Dickinson and Co.; Franklin Lakes, N.J., USA). After approximately 1 week, spheres with a clearly-defined three-dimensional structure consisting of >50 cells were quantified and used to calculate EOS. hsa-miR-34a treatment causes a 40% reduction in the clonogenic capacity of LAPC4 cells (Table 6) suggesting that hsa-miR-34a downregulation is important for CSC growth and that hsa-miR-34a has therapeutic benefit for the treatment of prostate cancer.

TABLE 6
hsa-miR-34a inhibits in vitro sphere-formation
in LAPC4 prostate tumor cells.

| | average number | | |
|---|---|---|---|
| miRNA | of spheres | SD | EOS |
| miR-NC | 110 | 18.38 | 100% |
| miR-34a | 66 | 15.56 | 60% |

Sphere formation of cells transfected with negative control miRNA was set at 100%. Abbreviations: EOS, efficiency of sphere formation; SD, standard deviation; miR-NC, negative control miRNA

### Example 6

### HSA-MIR-34A Inhibits Cancer Stem Cell Tumor Initiation in Vivo

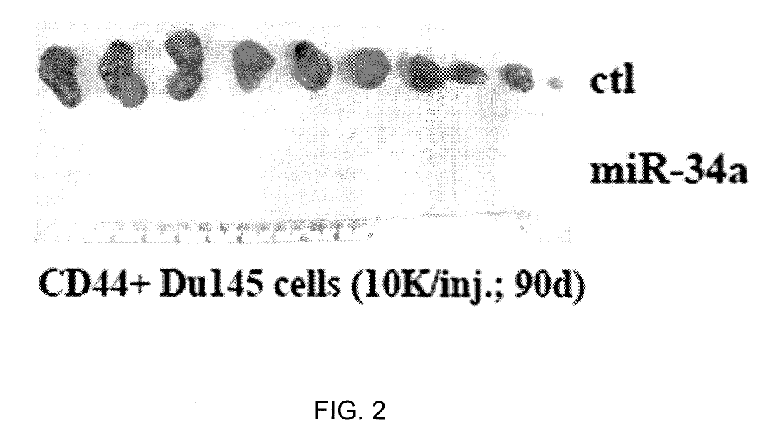

CSCs with the cell surface marker CD44 are more efficient than non-cancer stem cells in initiating tumors when transplanted into immunodeficient mice (Patrawala et al., 2005; 2006; 2007). Since hsa-miR-34a suppressed the in vitro clonogenic potential of the LAPC4 prostate cancer xenograft (Table 6 above), the inventors proceeded to determine if miR-34a could also interfere with CSC tumor-initiation in vivo. CD44+ Du145 prostate cancer cells cells were infected with a control lentivirus vector (lenti-ctl) or a lentivirus vector harboring pre-miR-34a (lenti-miR-34a) (Systems Biosciences Inc., Mountain View, Calif., USA), both at a multiplicity of infection (MOI) of 10. At 24 hr post infection, 10⁴ cells were mixed 1:1 with BD Matrigel (BD Biosciences)and implanted subcutaneuosly into the into the dorsal prostate of NOD/SCID mice (JAX® Mice and Services, Bar Harbor, Me., USA). One group of ten mice was injected with lenti-ctl-infected cells, and a separate group of ten animals was injected with lenti-mir-34a-infected cells. The mice were monitored for tumor growth for 90 days before sacrifice and assessment of tumor development. Tumors developed in all ten mice injected with cells that had been infected with lenti-ctl (ctl, FIG. 2). In contrast, none of the ten mice injected with cells that had been infected with lenti-miR-34a (miR-34a, FIG. 2) developed tumors during the experiment. Since the tumor incidence in the animal correlated with the presence of CD44⁺ cancer stem cells (Patrawala et al., 2006; Oncogene, 1696-708), this result demonstrated that hsa-miR-34a negatively impacted the CSCs' ability to generate tumors and that the miRNA can be used to treat the CSC component of tumors.

### Example 7

### Inhibition of HSA-MIR-34A Promotes Tumor Growth in Vivo

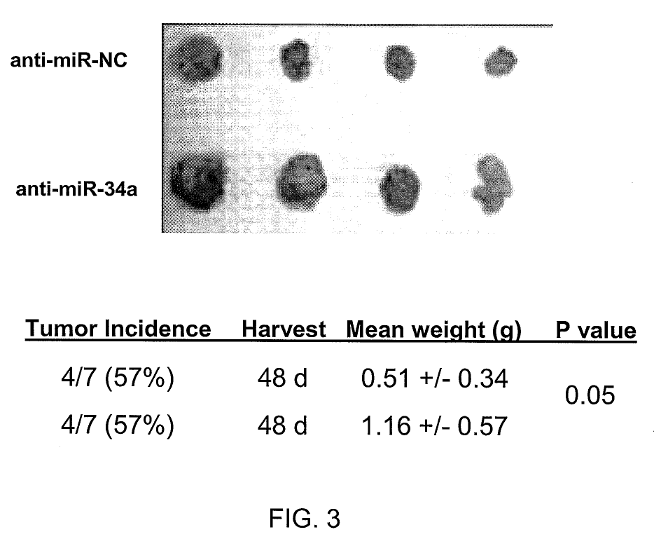

The inventors determined the effect of miR-34a inhibition on tumor initiation and tumor size in LAPC9 cells. Cells were transfected using Lipofectamine™ 2000 (Life Technologies;) with 33 nM of anti-miR-34a (Thermo Fisher Scientific Inc.; Lafayette, Colo., USA) or a negative control miRNA hairpin inhibitor negative control (Thermo Fisher Scientific Inc.), mixed 1:1 with BD Matrigel™, (BD Biosciences), and implanted into the dorsal prostate of intact male NOD/SCID mice (JAX® Mice and Services,). One group of seven mice was injected with anti-miR-34a -treated LAPC9 cells, and a separate group of seven animals was injected with LAPC9 cells (10⁵ cells) treated with negative control miRNA. After 48 days, animals were sacrificed and tumors were dissected, weighed, and imaged. Five of seven animals in each group developed tumors. A single administration of anti-miR-34a to LAPC9 prostate cancer cells, prior to tumor initiation in vivo, resulted in a 2 fold increase in average tumor weight compared with the anti-miR negative control-treated LAPC9 cells (FIG. 3). Similar results were obtained in experiments with LAPC4 cells. This supports the observation that cancer cells with lower miR-34a activity are more highly tumorigenic.

### Example 8

### Synthetic Mimics of MiRNAs Down Regulated in Cancer Stem Cells Inhibit Proliferation of Human Prostate Cancer Cells

The inventors assessed the anti-proliferative activity of multiple miRNAs (hsa-let-7b, hsa-miR-34a, hsa-miR-15a, hsa-miR-126, hsa-miR-16, hsa-miR-hsa-101, and hsa-miR-124a) that were down-regulated in at least one of the cancer stem cell populations that were used for expression analysis. Three individual human prostate cancer cell lines; PPC-1, derived from a bone metastasis; Du145, derived from a brain metastasis; and LNCaP, derived from a lymph node metastasis (Bello et al., 1997; Pretlow et al., 1993; Stone et al., 1978; Brothman et al., 1991; Horoszewicz et al., 1980). PPC-1 and Du145 cells lack expression of the prostate-specific antigen (PSA) and are independent of androgen receptor (AR) signaling. In contrast, LNCaP cells test positive for PSA and AR. Cells were transfected with synthetic miRNAs (Pre-miR™ miRNA Precursor Molecule; Life Technologies/Ambion, Austin, Tex., USA) or negative control miRNA (NC; Pre-miR™ miRNA Precursor Molecule-Negative Control #2; Ambion cat. no. AM17111) in a 96-well format using a lipid-based transfection reagent. Lipid-based reverse transfections were carried out in triplicate according to a published protocol (Ovcharenko et al., 2005) and the following parameters; (6,000-7,000 cells per each well of a 96 well plate), 0.1-0.2 μl Lipofectamine™ 2000 (cat. no. 11668-019, Life Technologies/Invitrogen Corp., Carlsbad, Calif., USA) in 20 μl OptiMEM (Life Technologies/Invitrogen), 30 nM final concentration of miRNA in 100 μl. Proliferation was assessed 4-7 days post-transfection using Alamar Blue™ (Life Technologies/Invitrogen) following the manufacturer's instructions. Fluorescent light units (FLU) were measured after 3 hours, normalized to the control, and plotted as percent change in proliferation. Percent proliferation of the miRNA-treated cells relative to cells treated with negative control miRNA (100%) is shown in Table 7.

TABLE 7
Percent (%) proliferation of human prostate cancer cell lines treated
with indicated miRNAs vs negative control miRNA (NC).

| | | |
|---|---|---|
| PPC-1 | LNCaP C4-2 | Du145 |

% Proliferation

| miRNA | Avg | SD | Avg | SD | Avg | SD |
|---|---|---|---|---|---|---|
| let-7b | 90.1 | 1.9 | 90.2 | 0.1 | 84.5 | 13.1 |
| 34a | 24.6 | 0.6 | 49.4 | 7.1 | 55.4 | 4.2 |
| 124a | 45.9 | 4.3 | 72.4 | 7.4 | 69.2 | 6.5 |
| 126 | 71.9 | 2.8 | 80.6 | 14.6 | 68.2 | 3.0 |
| 15a | 64.0 | 7.0 | 84.1 | 7.5 | 80.0 | 1.0 |
| 16 | 63.1 | 7.0 | 97.8 | 5.8 | 70.0 | 2.0 |
| 101 | 47.5 | 5.3 | 109.8 | 6.5 | 30.0 | 1.2 |

Values are normalized to values obtained from cells transfected with negative control miRNA (100% proliferation).
NC, negative control miRNA;
Avg, average;
SD, standard deviation.

Delivery of each synthetic miRNAs significantly inhibited cellular proliferation of human prostate cancer cells PPC-1, Du145, and LNCaP (Table 7). These results suggest that mimics of any of the miRNAs that are down-regulated in cancer stem cells might function as therapeutics for patients with prostate cancer and other malignancies.

### Example 9

### Repeated Dosing of Human Prostate Cancer Cells with Synthetic HSA-MIR-34A Inhibits Proliferation

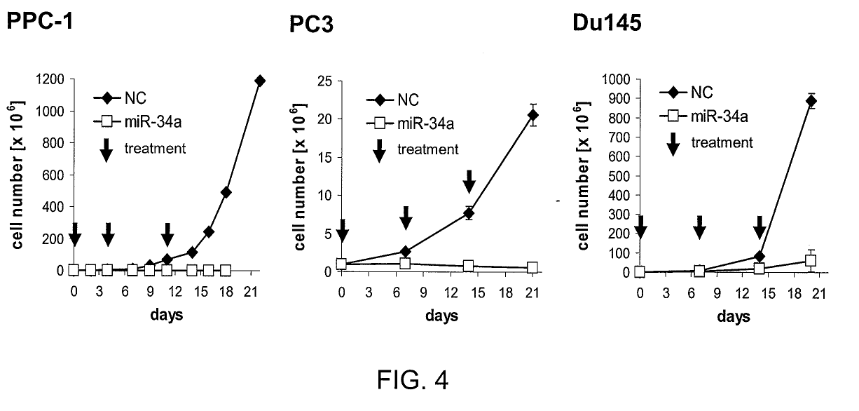

To evaluate the therapeutic activity of hsa-miR-34a over an extended period of time, growth curve experiments in the presence of miRNA for up to 22 days were conducted. Since in vitro transfections of naked interfering RNAs, such as synthetic miRNA, are transient by nature and compromised by the dilution of the oligo during ongoing cell divisions, miRNA was administered at multiple time points (Bartlett et al. 2006; Bartlett et al. 2007). To accommodate miRNA delivery into a large quantity of cells, the inventors employed the electroporation method to deliver hsa-miR-34a or negative control miRNA into PPC-1, PC3, and Du145 human prostate cancer cells. Briefly, 1×10⁶ PPC-1 or PC3 cells, or 0.5×10⁶ Du145 cells were electroporated with 1.6 μM hsa-miR-34a or negative control using the BioRad Gene Pulser Xcell™ instrument (BioRad Laboratories Inc., Hercules, Calif., USA), seeded and propagated in regular growth medium. Experiments with PC3 and Du145 cells were carried out in triplicates. When the control cells reached confluence (days 4 and 11 for PPC-1; days 7 and 14 for PC3 and Du145), cells were harvested, counted and electroporated again with the respective miRNAs. To ensure similar treatment of both conditions as well as to accommodate exponential growth, the cell numbers used for the second and third electroporation were titrated down to the lowest count. The population doubling was calculated from these electroporation events using the formula PD=ln(Nf/N0)/ln 2 and adjusting for the fact that approximately 72% of newly seeded cells adhere to the plate. Cell counts were extrapolated and plotted on a linear scale (FIG. 4). Arrows represent electroporation days. Standard deviations are included in the graphs.

Repeated administration of hsa-miR-34a robustly inhibited proliferation of human prostate cancer cells (FIG. 4, white squares). In contrast, cells treated with negative control miRNA showed normal exponential growth (FIG. 4, black diamonds). hsa-miR-34a treatment resulted in 97.2% inhibition of PC3 cell growth on day 21 (2.8% cells relative to cells electroporated with negative control miRNA), and 93.1% inhibition of Du145 cell growth on day 19 (6.9% cells relative to cells electroporated with negative control miRNA) relative to the proliferation of control cells (100%). All PPC-1 cells electroporated with hsa-miR-34a were eliminated by day 22.

The data indicate that hsa-miR-34a provides a useful therapeutic tool in the treatment of human prostate cancer cells.

### Example 10

### Synthetic HSA-MIR-34A Inhibits Tumor Growth of Human Prostate Cancer Xenografts in Mice

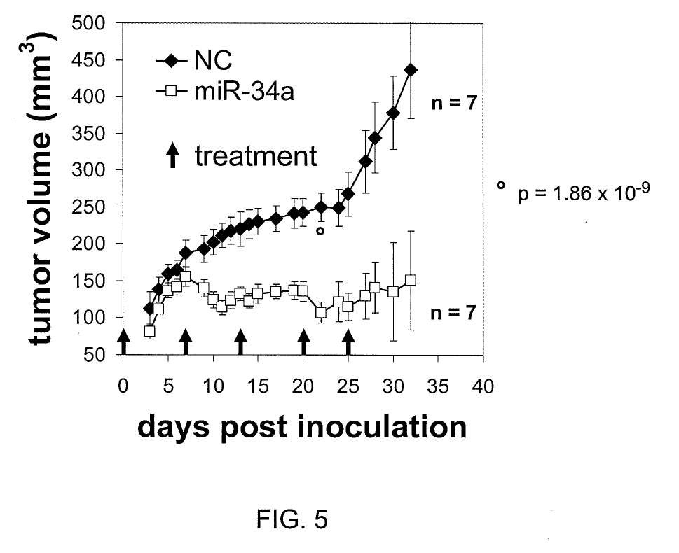

The therapeutic potential of synthetic hsa-miR-34a miRNA was evaluated in the animal using the PPC-1 human prostate cancer xenograft. 5×10⁶ PPC-1 cells per animal were electroporated with 1.6 μM synthetic hsa-miR-34a or negative control miRNA (Pre-miR™-hsa-miR-34a, Ambion cat. no. AM17100; NC, Pre-miR™ microRNA Precursor Molecule-Negative Control #2, Ambion cat. no. AM17111), mixed with BD Matrigel™, (BD Biosciences; San Jose, Calif., USA; cat. no. 356237) in a 1:1 ratio and implanted subcutaneously into the lower back of NOD/SCID mice (Charles River Laboratories, Inc.; Wilmington, Mass., USA). A group of 7 mice was injected with hsa-miR-34a treated PPC-1 cells, and a group of 7 animals was injected with PPC-1 cells treated with negative control miRNA. To maintain steady levels of miRNA, 6.25 μg of each hsa-miR-34a or negative control miRNA conjugated with the lipid-based siPORT™ amine delivery agent (Ambion, Austin, Tex.; cat. no. AM4502) were repeatedly administered on days 7, 13, 20, and 25 via intra-tumoral injections. Given an average mouse weight of 20 g, this dose equals 0.3125 mg/kg. Tumor growth was monitored by taking caliper measurements every 1-2 days for 32 days. Tumor volumes were calculated using the formula, Volume=length×width×width/2, in which the length is greater than the width, and plotted over time (FIG. 5). Standard deviations are shown in the graph. All data points had p values <0.01. p values were as low as 1.86×10⁻⁹ for data obtained on day 22, indicating statistical significance.

Repeated dosing with hsa-miR-34a blocked tumor growth of the human PPC-1 prostate cancer xenograft (FIG. 5, white squares). The average volume of tumors that received hsa-miR-34a was 151 mm³ on day 32. Volumes of newly implanted tumors ranged between 111 and 155 mm³ (days 4-7) and thus, PPC-1 tumors failed to develop in response to hsa-miR-34a treatment. In contrast, tumors locally treated with negative control miRNA were unaffected and continued to grow at a steady pace (FIG. 5, black diamonds). The average volume of tumors treated with negative control miRNA was 437 mm³ on day 32. Of note, each single administration with hsa-miR-34a resulted in an acute regression of tumor volumes. This effect was not induced with negative control miRNA, indicating that the anti-tumor activity of hsa-miR-34a is specific.

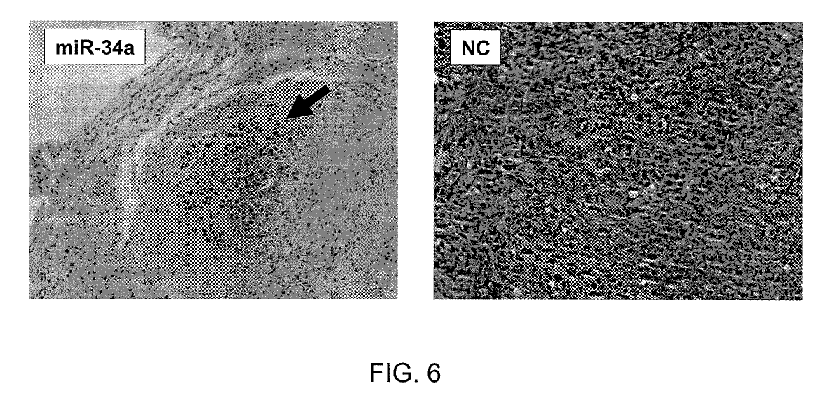

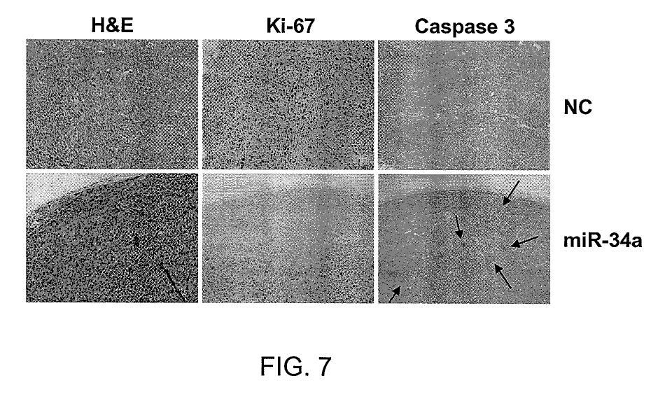

A histological analysis revealed that PPC-1 tumors treated with negative control miRNA were densely packed with healthy, viable prostate cancer cells (FIG. 6). In contrast, hsa-miR-34a-treated tumors consisted mostly of matrigel with cellular debris and sparsely distributed cells, as well as occasional pockets with seemingly viable cells (FIG. 6, arrow). To gain further insight into the biological status of these cells, immunohistochemistry analyses specific for the proliferation marker Ki-67, as well as caspase 3, an indicator of apoptosis were performed. As illustrated in FIG. 7, areas with viable cells in hsa-miR-34a-treated tumors showed reduced levels of Ki-67 and increased levels of caspase 3. The data indicate that hsa-miR-34a inhibits tumor growth by an anti-proliferative and pro-apoptotic mechanism.

The data indicate that hsa-miR-34a provides a powerful therapeutic tool in the treatment of patients with prostate cancer.

In addition, the data demonstrated the therapeutic utility of hsa-miR-34a in a lipid-based formulation.

### Example 11

### Delivery of Synthetic HSA-MIR-34A Decreases Metastasis and Increases Survival in Mice with Prostatecancer

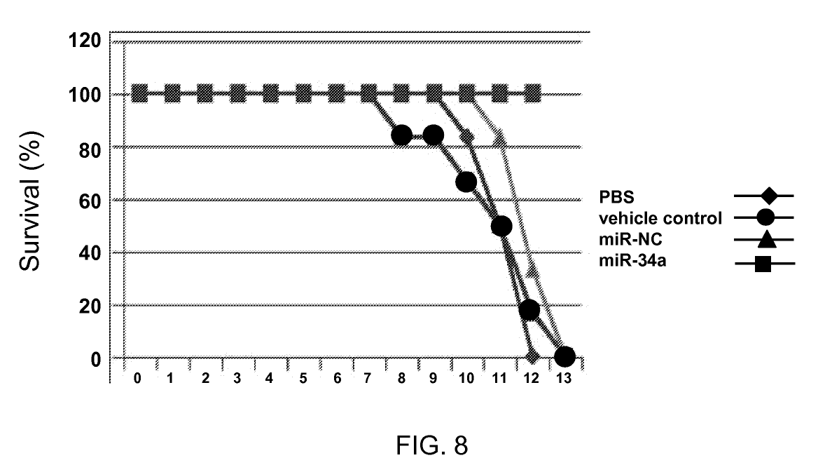

To evaluate the therapeutic benefit of treating cancer patients with a miRNA that is down-regulated in cancer stem cells, we injected mice that had orthotopic prostate tumors with synthetic hsa-miR-34a at two day intervals with a dose of 1 mg of miRNA/kg body weight. LAPC9-GFP cells (500,000) were orthotopically implanted in the DP of male NOD/SCID mice. Twenty-one days later, animals were divided into four groups (n=7 for each group) and injected, via tail vein, with the indicated materials (PBS, vehicle control; miR-NC; and miR-34a) on day 0. Injections were repeated on days 2, 4, 6, 8, and 10 and all animals were terminated on day 13. Animals were tracked for survival before being sacrificed for analysis. As shown in FIG. 8, all of the control mice had died by day 13 of the experiment and none of the miR-34a treated animals died. Lung imaging revealed significantly reduced lung metastases in the miR-34a treated group compared to the NC group.

These data indicate that hsa-miR-34a can significantly enhance the survival of cancer patients by limiting metastasis.

### Example 12

### Synthetic MiRNAs Inhibit Proliferation of Human Lung Cancer Cells

miRNAs with therapeutic value in prostate cancer and prostate cancer stem cells may also be of therapeutic value in other cancer types. Therefore, the inventors assessed the therapeutic effect of several miRNAs, down-regulated in CSCs, for lung cancer by using four individual lung cancer cell lines. To measure cellular proliferation of lung cancer cells, the following non-small cell lung cancer (NSCLC) cells were used; cells derived from lung adenocarcinoma (A549, Calu-3, HCC2935) and cells derived from lung large cell carcinoma (H460). Synthetic miRNAs (Pre-miR™, Life Technologies/Ambion) or negative control (NC) miRNA (Pre-miR™ miRNA Precursor Molecule-Negative Control #2; Ambion cat. no. AM17111) was delivered via lipid-based transfection into A549, Calu-3, HCC2935, and H460 cells.

Lipid-based reverse transfections were carried out in triplicate according to a published protocol (Ovcharenko et al., 2005) and the following parameters: cells (5,000-12,000 per each well of 96 well plate), 0.1-0.2 μl Lipofectamine™ 2000 (cat. no. 11668-019, Invitrogen Corp., Carlsbad, Calif., USA) in 20 μl OptiMEM (Invitrogen), 30 nM final concentration of miRNA in 100 μl. All cells except for Calu-3 cells were harvested 72 hours post transfection or electroporation for assessment of cellular proliferation. Calu-3 cells were harvested 10 days post transfection. Proliferation assays were performed using Alamar Blue (Invitrogen) following the manufacturer's instructions. Percent (%) proliferation values from the Alamar Blue assay were normalized to values from cells treated with negative control miRNA. Percent proliferation of miRNA-treated cells relative to cells treated with negative control miRNA (100%) is shown in Table 8.

TABLE 8
Percent (%) proliferation of lung cancer cell lines treated with
mimics of miRNAs down-regulated in cancer stem cells.

| | A549 | | H460 | | Calu-3 | | HCC2935 | |
|---|---|---|---|---|---|---|---|---|
| | % Proliferation | | | | | | | |
| miRNA | Avg | SD | Avg | SD | Avg | SD | Avg | S D |
| let-7b | 68 | 11 | 32 | 3 | 71 | 4 | 80 | 3 |
| miR-126 | 84 | 4 | 33 | 3 | 66 | 4 | 68 | 7 |
| miR-124a | 70 | 12 | 18 | 4 | 28 | 5 | 76 | 7 |
| miR-34a | 76 | 6 | 47 | 8 | 32 | 8 | 74 | 6 |

Values are normalized to values obtained from cells transfected with negative control miRNA (100% proliferation).
Avg, average;
SD, standard deviation.

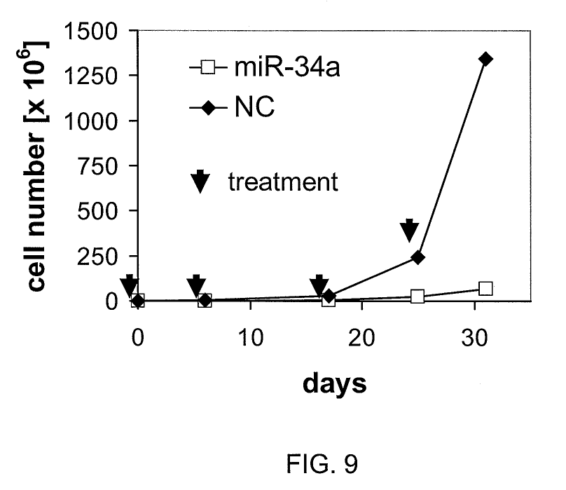

To evaluate the therapeutic activity of hsa-miR-34a over an extended period of time, the inventors conducted growth curve experiments in the presence of miRNA for up to 31 days in H226 lung cancer cells. Since in vitro transfections of naked interfering RNAs, such as synthetic miRNA, are transient by nature and compromised by the dilution of the miRNA during ongoing cell divisions, miRNA was administered at multiple time points (Bartlett et al., 2006; Bartlett et al., 2007). To accommodate miRNA delivery into a large quantity of cells, hsa-miR-34a or negative control miRNA were delivered by the electroporation method. Briefly, 1×10⁶ H226 were electroporated in triplicate with 1.6 μM hsa-miR-34a or negative control using the BioRad Gene Pulser Xcell™ instrument (BioRad Laboratories Inc., Hercules, Calif., USA), seeded and propagated in regular growth medium. When the control cells reached confluence (days 6, 17, and 25), cells were harvested, counted and electroporated again with the respective miRNAs. To ensure similar treatment of both conditions as well as to accommodate exponential growth, the cell numbers used for the second and third electroporation were titrated down to the lowest count. The population doubling was calculated from these electroporation events using the formula PD=ln(Nf/NO)/ln 2 and adjusting for the fact that approximately 72% of newly seeded cells adhere to the plate. Cell counts were extrapolated and plotted on a linear scale (FIG. 9). Arrows represent electroporation days. Standard deviations are included in the graphs.

Repeated administration of hsa-miR-34a robustly inhibited proliferation of human lung cancer cells (FIG. 9). In contrast, cells treated with negative control miRNA showed normal exponential growth. hsa-miR-34a treatment resulted in 94.9% inhibition of H226 cell growth on day 31 (5.1% remaining cells) relative to the proliferation of control cells (100%).

The data indicate that miRNAs that are down-regulated cancer stem cells can be used as therapeutics to treat additional cancers including lung cancer.

### Example 13

### Synthetic HSA-MIR-34A Inhibits Tumor Growth of Human Lung Cancer Xenografts in Mice

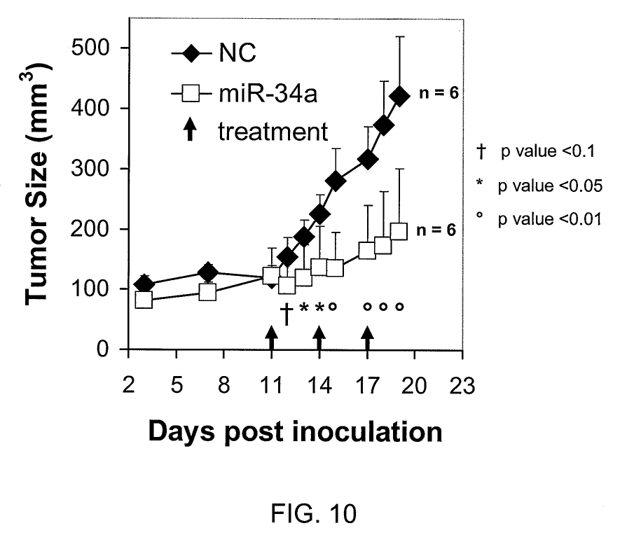

The inventors assessed the growth-inhibitory activity of hsa-miR-34a in human lung cancer xenografts grown in immunodeficient mice. Each 3×10⁶ human H460 non-small cell lung cancer cells were mixed with BD Matrigel™, (BD Biosciences; San Jose, Calif., USA; cat. no. 356237) in a 1:1 ratio and subcutaneously injected into the lower back of 23 NOD/SCID mice (Charles River Laboratories, Inc.; Wilmington, Mass., USA). Once animals developed palpable tumors (day 11 post xenograft implantation), a group of six animals received intratumoral injections of each 6.25 μg hsa-miR-34a (Dharmacon, Lafayette, Colo.) formulated with the lipid-based siPORT™ amine delivery agent (Life Technologies/Ambion; cat. no. AM4502) on days 11, 14, and 17. A control group of six animals received intratumoral injections of each 6.25 μg negative control miRNA (NC; Dharmacon, Lafayette, Colo.), following the same injection schedule that was used for hsa-miR-34a. Given an average mouse weight of 20 g, this dose equals 0.3125 mg/kg. In addition, a group of six H460 tumor-bearing mice received intratumoral injections of the siPORT™ amine delivery formulation lacking any oligonucleotide, and a group of five animals received intratumoral injections of phosphate-buffered saline (PBS). Caliper measurements were taken every 1-2 days, and tumor volumes were calculated using the formula, Volume=length×width×width/2, in which the length is greater than the width. Average tumor volumes, standard deviations and p-values were calculated and plotted over time (FIG. 10).

As shown in FIG. 10, three doses of hsa-miR-34a robustly inhibited growth of established H460 lung tumors (white squares). On day 19, the average volume of tumors treated with hsa-miR-34a was 196 mm³. In contrast, tumors treated with negative control miRNA (black diamonds) grew at a steady pace and yielded tumors with an average size of 421 mm³ on day 19. Negative control tumors developed as quickly as tumors treated with either PBS or the siPORT amine only control, indicating that the therapeutic activity of hsa-miR-34a is specific.

The data indicate that hsa-miR-34a represents a particularly useful candidate in the treatment of patients with lung cancer. The data also indicate that hsa-miR-34a provides universal therapeutic activity against a broad spectrum of cancer types and cancer stem cells. The therapeutic activity of hsa-miR-34a is highlighted by the fact that hsa-miR-34a inhibited growth of tumors that had developed prior to treatment.

In addition, the data demonstrate the therapeutic utility of hsa-miR-34a in a lipid-based formulation.

### Example 14

### Elivery of Synthetic HSA-MIR-126 Inhibits Tumor Growth of Human Prostate Cancer Xenografts in Mice

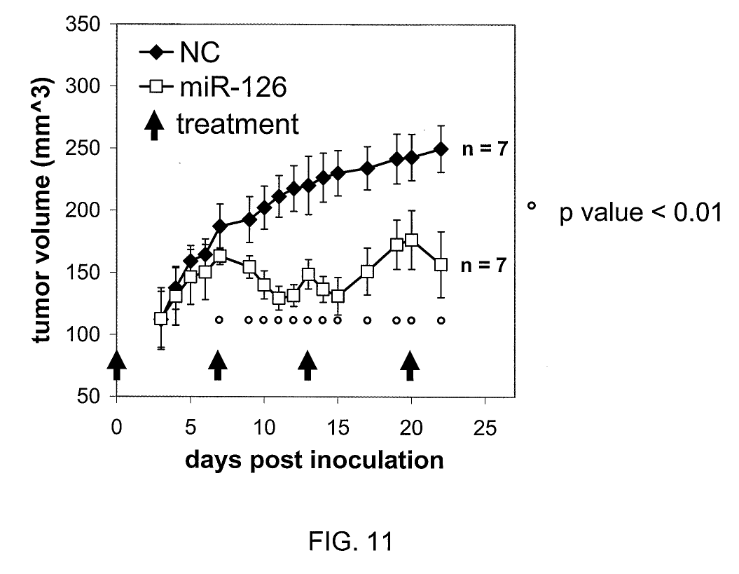

The in vitro studies demonstrated the therapeutic activity of hsa-miR-126 in cultured human prostate cancer cells. Therefore, hsa-miR-126 is likely to interfere with prostate tumor growth in the animal. To explore this, the therapeutic potential of synthetic hsa-miR-126 miRNA was evaluated in mice using the PPC-1 human prostate cancer xenograft. PPC-1 cells, 5×10⁶ per animal, were electroporated with 1.6 μM synthetic hsa-miR-126 or negative control miRNA (Pre-miR™-hsa-miR-126, Ambion cat. no. AM17100; NC, Pre-miR™ miRNA Precursor Molecule-Negative Control #2, Ambion cat. no. AM17111), mixed with BD Matrigel™, (BD Biosciences; cat. no. 356237) in a 1:1 ratio and implanted subcutaneously into the lower back of NOD/SCID mice (Charles River Laboratories, Inc.; Wilmington, Mass., USA). A group of 7 mice was injected with hsa-miR-126 treated PPC-1 cells, and a group of 7 mice was injected with PPC-1 cells treated with negative control miRNA. To maintain steady levels of miRNA, 6.25 μg of hsa-miR-126 or negative control miRNA conjugated with the lipid-based siPORT™ amine delivery agent (Ambion; cat. no. AM4502) were repeatedly administered on days 7, 13 and 20 via intra-tumoral injections. Given an average mouse weight of 20 g, this dose equals 0.3125 mg/kg. Tumor growth was monitored by taking caliper measurements every 1-2 days for 22 days. Tumor volumes were calculated using the formula, Volume=length×width×width/2, in which the length is greater than the width, and plotted over time (FIG. 11). Standard deviations and data points with p values <0.01, indicating statistical significance, are shown in the graph.

Repeated dosing with hsa-miR-126 blocked tumor growth of the human PPC-1 prostate cancer xenograft (FIG. 11). The average volume of tumors that received hsa-miR-126 was 157 mm³ on day 22. In contrast, tumors locally treated with negative control miRNA were unaffected and continued to grow at a steady pace. The average volume of tumors treated with negative control miRNA was 250 mm³ on day 22. Of note, each single administration with hsa-miR-126 resulted in an acute regression of tumor volumes. This effect was not induced with negative control miRNA, indicating that the anti-tumor activity of hsa-miR-126 is specific. In response to hsa-miR-126 treatments on days 7 and 13, tumor volumes diminished to approximately 130 mm³, a size that is comparable with newly implanted and hardly detectable tumors (day 4).

The data suggest that hsa-miR-126 provides a powerful therapeutic tool in the treatment of patients with prostate cancer. In addition, the data demonstrated the therapeutic utility of hsa-miR-126 in a lipid-based formulation.

### Example 15

### Delivery of Synthetic HSA-MIR-126 Inhibits Tumor Growth of Human Lung Cancer Xenografts in Mice

Delivery of hsa-miR-126 inhibited the growth of cultured human lung cancer cells. These results demonstrated the anti-oncogenic activity of hsa-miR-126 and suggested that hsa-miR-126 may also provide a powerful therapeutic tool to treat established lung tumors in the animal. To explore this possibility, each 3×10⁶ human H460 non-small lung cancer cells were mixed with BD Matrigel™, (BD Biosciences; San Jose, Calif., USA; cat. no. 356237) in a 1:1 ratio and subcutaneously injected into the lower back of 23 NOD/SCID mice (Charles River Laboratories, Inc). Once animals developed palpable tumors (day 11 post xenograft implantation), a group of six animals received intratumoral injections of each 6.25 μg hsa-miR-126 (Dharmacon, Inc., Lafayette, Colo., USA) formulated with the lipid-based siPORT™ amine delivery agent (Ambion, Austin, Tex.; cat. no. AM4502) on days 11, 14 and 17. A control group of six animals received intratumoral injections of each 6.25 μg negative control miRNA (NC; Dharmacon, Lafayette, Colo., USA), following the same injection schedule that was used for hsa-miR-126. Given an average mouse weight of 20 g, this dose equals 0.3125 mg/kg. In addition, a group of six H460 tumor-bearing mice received intratumoral injections of the siPORT™ amine delivery formulation lacking any oligonucleotide, and a group of five animals received intratumoral injections of phosphate-buffered saline (PBS). Caliper measurements were taken every 1-2 days, and tumor volumes were calculated using the formula, Volume=length×width×width/2, in which the length is greater than the width.

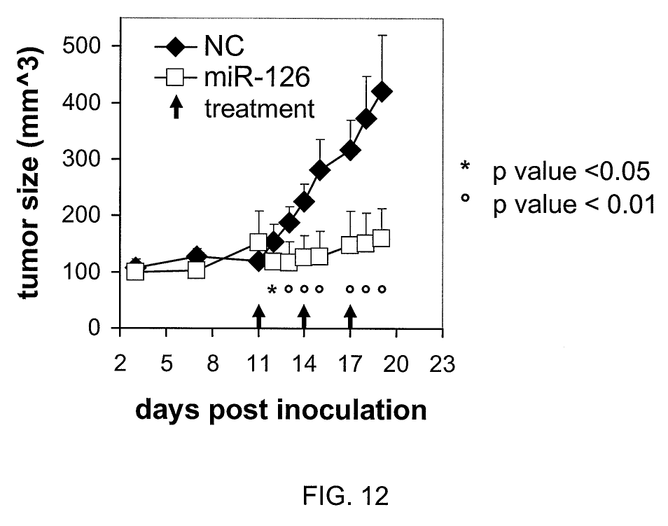

As shown in FIG. 12, three doses of hsa-miR-126 robustly inhibited growth of established H460 lung tumors. In contrast, tumors treated with negative control miRNA grew at a steady pace and yielded tumors with an average size of 420 mm³ on day 19. Negative control tumors developed as quickly as tumors treated with either PBS or the siPORT amine only control, indicating that the therapeutic activity of hsa-miR-126 is specific.

The data suggest that hsa-miR-126 represents a particularly useful candidate in the treatment of patients with lung cancer. The data also indicated that hsa-miR-126 provides universal therapeutic activity against a broad spectrum of cancer types and cancer stem cells. The therapeutic activity of hsa-miR-126 is highlighted by the fact that hsa-miR-126 inhibited tumor growth of tumors that had developed prior to treatment. In addition, the data demonstrated the therapeutic utility of hsa-miR-126 in a lipid-based formulation.

### Example 16

### Delivery of Synthetic HSA-LET-7 Inhibits Tumor Growth of Human Lung Cancer Xenografts in Mice

Delivery of hsa-miR-let-7 inhibited the growth of cultured human lung cancer cells. These results demonstrated the anti-oncogenic activity of hsa-let-7 and suggest that hsa-let-7 may also provide a powerful therapeutic tool to treat established lung tumors in the animal. To explore this possibility, each 3×10⁶ human H460 non-small lung cancer cells were mixed with BD Matrigel™, (BD Biosciences; cat. no. 356237) in a 1:1 ratio and subcutaneously injected into the lower back of 23 NOD/SCID mice (Charles River Laboratories, Inc.). Once animals developed palpable tumors (day 11 post xenograft implantation), a group of six animals received intratumoral injections of each 6.25 μg hsa-let-7b (Dharmacon, Inc.) formulated with the lipid-based siPORT™ amine delivery agent (Ambion; cat. no. AM4502) on days 11, 14 and 17. A control group of six animals received intratumoral injections of each 6.25 μg negative control miRNA (NC; Dharmacon, Lafayette, Colo.), following the same injection schedule that was used for hsa-let-7b. Given an average mouse weight of 20 g, this dose equals 0.3125 mg/kg. In addition, a group of six H460 tumor-bearing mice received intratumoral injections of the siPORT™ amine delivery formulation lacking any oligonucleotide, and a group of five animals received intratumoral injections of phosphate-buffered saline (PBS). Caliper measurements were taken every 1-2 days, and tumor volumes were calculated using the formula, Volume=length×width×width/2, in which the length is greater than the width.

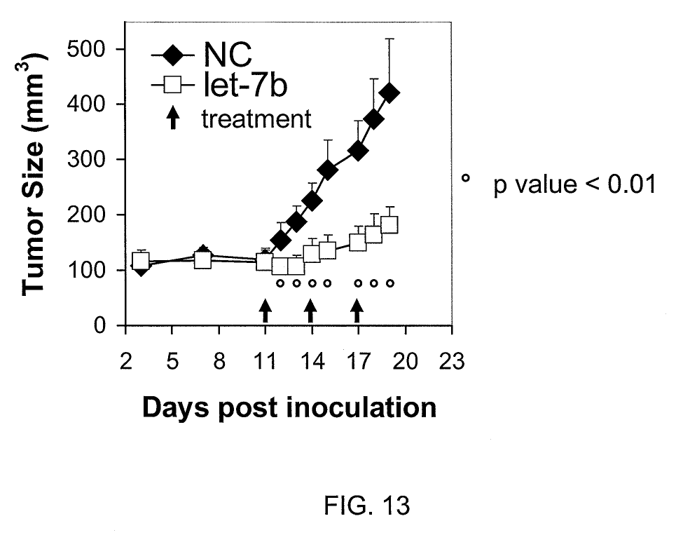

As shown in FIG. 13, three doses of hsa-let-7b robustly inhibited growth of established H460 lung tumors. In contrast, tumors treated with negative control miRNA grew at a steady pace and yielded tumors with an average size of 420 mm³ on day 19. Negative control tumors developed as quickly as tumors treated with either PBS or the siPORT amine only control, indicating that the therapeutic activity of hsa-let-7b is specific.

The data suggest that hsa-let-7b represents a particularly useful candidate in the treatment of patients with lung cancer. The therapeutic activity of hsa-let-7b is highlighted by the fact that hsa-let-7b inhibited growth of tumors that had developed prior to treatment. In addition, the data demonstrated the therapeutic utility of hsa-let-7b in a lipid-based formulation.

Hsa-miR-34a, hsa-miR-126, and hsa-let-7 are among the miRNAs that are downregulated in prostate cancer stem cells. Reintroduction of these miRNAs provides a therapeutic response in multiple models of human cancer, such as prostate and lung cancer. The data indicate that miRNAs downregulated in prostate cancer stem cells have therapeutic value against a broad spectrum of cancer types and cancer stem cells.

### REFERENCES

The following references, to the extent that they provide exemplary procedural or other details supplementary to those set forth herein, are specifically incorporated herein by reference.
U.S. Pat. No. 4,659,774
U.S. Pat. No. 4,682,195
U.S. Pat. No. 4,683,202
U.S. Pat. No. 4,704,362
U.S. Pat. No. 4,816,571
U.S. Pat. No. 4,870,287
U.S. Pat. No. 4,959,463
U.S. Pat. No. 5,141,813
U.S. Pat. No. 5,143,854
U.S. Pat. No. 5,202,231
U.S. Pat. No. 5,221,619
U.S. Pat. No. 5,242,974
U.S. Pat. No. 5,264,566
U.S. Pat. No. 5,288,644
U.S. Pat. No. 5,324,633
U.S. Pat. No. 5,384,261
U.S. Pat. No. 5,399,363
U.S. Pat. No. 5,405,783
U.S. Pat. No. 5,411,876
U.S. Pat. No. 5,412,087
U.S. Pat. No. 5,413,924
U.S. Pat. No. 5,424,186
U.S. Pat. No. 5,428,148
U.S. Pat. No. 5,429,807
U.S. Pat. No. 5,432,049
U.S. Pat. No. 5,436,327
U.S. Pat. No. 5,445,934
U.S. Pat. No. 5,466,468
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,543,158
U.S. Pat. No. 5,545,531
U.S. Pat. No. 5,547,839
U.S. Pat. No. 5,550,044
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,580,726
U.S. Pat. No. 5,580,732
U.S. Pat. No. 5,583,013
U.S. Pat. No. 5,593,839
U.S. Pat. No. 5,599,672
U.S. Pat. No. 5,599,695
U.S. Pat. No. 5,602,244
U.S. Pat. No. 5,610,287
U.S. Pat. No. 5,624,711
U.S. Pat. No. 5,631,134
U.S. Pat. No. 5,639,603
U.S. Pat. No. 5,641,515
U.S. Pat. No. 5,645,897
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,695,940
U.S. Pat. No. 5,700,637
U.S. Pat. No. 5,705,629
U.S. Pat. No. 5,739,169
U.S. Pat. No. 5,744,305
U.S. Pat. No. 5,760,395
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,846,225
U.S. Pat. No. 5,846,945
U.S. Pat. No. 5,847,219
U.S. Pat. No. 5,871,928
U.S. Pat. No. 5,876,932
U.S. Pat. No. 5,919,626
U.S. Pat. No. 5,985,619
U.S. Pat. No. 6,004,755
U.S. Pat. No. 6,087,102
U.S. Pat. No. 6,368,799
U.S. Pat. No. 6,383,749
U.S. Pat. No. 6,403,341
U.S. Pat. No. 6,617,112
U.S. Pat. No. 6,638,717
U.S. Pat. No. 6,720,138
U.S. Pat. No. 6,723,509
U.S. Patent Appln. Prov. Ser. 60/650,807
U.S. Patent Appln. Prov. Ser. 60/869,295
U.S. patent application Ser. No. 10/667,126
U.S. patent application Ser. No. 11/141,707
U.S. patent application Ser. No. 11/273,640
U.S. patent application Ser. No. 11/349,727
U.S. patent application Ser. No. 11/567,082
U.S. patent application Ser. No. 11/855,792,
U.S. patent application Ser. No. 11/857,948
Abdulghani et al., Am. J. Pathol., 172(6):1717-28, 2008.
Al-Hajj et al., Proc. Natl. Acad. Sci. USA, 100(7):3983-8, 2003.
Andersen et al., J. Clin. Oncol., 22: 3570-3580, 2004.
Arap et al., Cancer Res., 55(6):1351-1354, 1995.
Austin-Ward and Villaseca, Revista Medica de Chile, 126(7):838-845, 1998.
Bagga et al., Cell, August 26;122(4):553-63. 2005
Bao et al., Nature, 444(7120):756-60, 2006.
Beier et al., Cancer Res., 67(9):4010-5, 2007.
Ben-Porath et al., Nat. Genet., 40(5):499-507, 2008.
Berman et al., Science, 297(5586):1559-61, 2002.
Birnie et al., Genome Biol., 9(5):R83. [Epub ahead of print], 2008.
Bonnet and Dick, Nat. Med., 3(7):730-7, 1997.
Bourguignon et al., J. Biol. Chem., 283(25): 17635-51, 2008.
Bukowski et al., Clinical Cancer Res., 4(10):2337-2347, 1998.
Caldas et al., Cancer Res., 54:3568-3573, 1994.
Carrington et al. Science, 301(5631):336-338, 2003.
Chen et al., Curr. Gene Ther., 5(1):71-80, 2005.
Cheng et al., Cancer Res., 54(21):5547-5551, 1994.
Christodoulides et al., Microbiology, 144(Pt 11):3027-3037, 1998.
Clarke et al., Cancer Res., 66(19):9339-44, 2006.
Clement et al., Curr. Biol., 17(2): 165-72, 2007
Collins et al., Cancer Res., 65(23):10946-51, 2005.
Cummins et al., In: IRT: Nucleosides and nucleosides, La Jolla Calif., 72, 1996.
Dai et al., Cancer Res., 68(14): 5785-94, 2008.
Davidson et al., J. Immunother., 21(5):389-398, 1998.
Denli et al., Trends Biochem. Sci., 28:196, 2003.
Didenko, Biotechniques, 31(5):1106-1116, 1118, 1120-1121, 2001.
Dillman, Cancer. Biother. Radiopharm., 14(1):5-10, 1999.
Dontu et al., Genes Dev., 17:1253-70, 2003.
Doyle and Ross, Oncogene, 22(47):7340-58, 2003.
Dylla et al., PLoS One, 3(6):e2428, 2008.
Emptage et al., Neuron, 29(1):197-208, 2001.
Engelmann et al., Cancer Res., 68(7):2419-26, 2008.
EP 266,032
EP 373 203
EP 785 280
EP 799 897
Fan et al., Cancer Res., 66(15): 7445-52, 2006.
Fan et al., Endocrinology, 145: 3961-3970, 2004.
Francipane et al., Cancer Res., 68 (11):4022-4025, 2008.
Froehler et al., Nucleic Acids Res., 14:5399-5407, 1986.
Ginestier et al., Cell Stem Cell, 1(5):555-567, 2007.
Goodell et al., J. Exp. Med., 183(4):1797-806, 1996.
Griffey et al., J. Mass Spectrom, 32(3):305-13, 1997.
Griffiths-Jones et al., Nucl. Acids Res., 36(Database Issue):D154-D158, 2008.
Gu et al., Cancer Res., 67(10):4807-15, 2007.
Guan and Hogge, Leukemia, 14(12):2135-41, 2000.
Hambardzumyan et al., Genes Dev., 22(4):436-48, 2008.
Hanibuchi et al., Int. J. Cancer, 78(4):480-485, 1998.
Hellstrand et al., Acta Oncologica, 37(4):347-353, 1998.
Hermann et al., Cell Stem Cell, 1(3):313-23, 2007.
Hirschmann-Jax et al., Proc. Natl. Acad. Sci. USA, 101:14228-33, 2004.
Ho et al., Exp. Hematol., 36(4): 433-42, 2008.
Holyaoke et al., Blood, 94(6):2056-64, 1999.
Hui and Hashimoto, Infection Immun., 66(11):5329-5336, 1998.
Hurt et al., Br. J. Cancer, 98(4):756-65, 2008.
Hussussian et al., Nat. Genet., 8(1):15-21, 1994.
Itakura and Riggs, Science, 209:1401-1405, 1980.
Jamieson et al., N. Engl. J. Med., 351(7):657-67, 2004.
Jin et al., Nat Med., 12(10):1167-74. 2006.
Ju et al., Gene Ther., 7(19):1672-1679, 2000.
Kamb et al., Nat. Genet., 8(1):23-26, 1994.
Kamb et al., Science, 2674:436-440, 1994.
Karhadkar et al., Nature, 431(7009):707-12, 2004.
Keshet et al., Biochem. Biophys. Res. Commun., 368(4):930-6, 2008.
Klostermeier and Millar, Biopolymers, 61(3):159-79, 2001-2002.
Konopleva et al., Cancer Cell,10(5):375-88, 2006.
Lechner et al., Biochem. Biophys. Res. Commun., 293(2):670-674, 2002.
Lee et al., EMBO J., 21(17):4663-70, 2002
Leong & Gao, Differentiation, 76(6): 699-716, 2008.
Lessard and Sauvageau, Nature, 423(6937):255-60, 2003.
Li et al., Cancer Biol. Ther., 6(9):1480-9, 2007.
Li et al., J. Natl. Cancer Inst., 100(9):672-9, 2008.
Li et al., Oncogene, 2008. [Epub ahead of print]
Li et al., Proc Natl Acad Sci USA, 100(26):15853-8, 2003.
Lim et al., Nature, 433(7027):769-73, 2005.
Liu et al., Cancer Res., 66(8):4011-9, 2006.
Liu et al., Cancer Res., 66(12):6063-71, 2006.
Liu et al., Mol. Cancer, 5:67, 2006.
Liu et al., N. Engl. J. Med., 356(3):217-26, 2007.
Lu et al., Proc. Natl. Acad. Sci. USA, 103(15): 5688-93, 2006.
Malanchi et al., Nature, 452(7187):650-3, 2008.
Marsters et al., Recent Prog. Horm. Res., 54:225-234, 1999.
Miki et al., Cancer Res., 67(7):3153-61, 2007.
Mori et al., Cancer Res., 54(13):3396-3397, 1994.
Nobori et al., Nature (London), 368:753-756, 1995.
Okamoto et al., Proc. Natl. Acad. Sci. USA, 91(23):11045-11049, 1994.
Olsen et al., Dev. Biol., 216:671, 1999.
Orlow et al., Cancer Res., 54(11):2848-2851, 1994.
Patrawala et al., Cancer Res., 65(14):6207-19, 2005.
Patrawala et al., Cancer Res., 67(14):6796-805, 2007.
Patrawala et al., Oncogene, 25(12):1696-708, 2006.
PCT Appln. WO 0138580
PCT Appln. WO 0168255
PCT Appln. WO 03020898
PCT Appln. WO 03022421
PCT Appln. WO 03023058
PCT Appln. WO 03029485
PCT Appln. WO 03040410
PCT Appln. WO 03053586
PCT Appln. WO 03066906
PCT Appln. WO 03067217
PCT Appln. WO 03076928
PCT Appln. WO 03087297
PCT Appln. WO 03091426
PCT Appln. WO 03093810
PCT Appln. WO 03100012
PCT Appln. WO 03100448A1
PCT Appln. WO 04020085
PCT Appln. WO 04027093
PCT Appln. WO 09923256
PCT Appln. WO 09936760
PCT Appln. WO 93/17126
PCT Appln. WO 95/11995
PCT Appln. WO 95/21265
PCT Appln. WO 95/21944
PCT Appln. WO 95/21944
PCT Appln. WO 95/35505
PCT Appln. WO 96/31622
PCT Appln. WO 97/10365
PCT Appln. WO 97/27317
PCT Appln. WO 9743450
PCT Appln. WO 99/35505
Peacock et al., Proc. Natl. Acad. Sci. USA, 104(10):4048-53, 2007.
Peltier & Latham, RNA, 14(5): 844-52, 2008.
Pietras et al., Oncogene, 17(17):2235-2249, 1998.
Qin et al., Proc. Natl. Acad. Sci. USA, 95(24):14411-14416, 1998.
Reiters and Sawyers, In: Prostate Cancer: Biology, Genetics, and the New Therapeutics, Totowa, N.J., 163-173, 2001.
Remington's Pharmaceutical Sciences 15^{th} Ed., 1035-1038 and 1570-1580, 1990.
Reynolds et al., Science, 255: 1707-1710, 1992.
Richardson et al., J. Cell Sci., 117(Pt 16):3539-45, 2004.
Sambrook et al., In: DNA microaarays: a molecular cloning manual, Cold Spring Harbor Laboratory Press, Cold Spring Harbor, N.Y., 2003.
Sambrook et al., In: Molecular cloning, Cold Spring Harbor Laboratory Press, Cold Spring Harbor, N.Y., 2001.
Sambrook et al., In: Molecular cloning: a laboratory manual, 2^{nd} Ed., Cold Spring Harbor Laboratory Press, Cold Spring Harbor, N.Y., 1989.
Seggerson et al., Dev. Biol., 243:215, 2002.
Serrano et al., Nature, 366:704-707, 1993.
Serrano et al., Science, 267(5195):249-252, 1995.
Shepherd et al., Prostate, 68(9):1007-1024, 2008.
Shipitsin et al., Cancer Cell, 11(3):259-73, 2007.
Singh et al., Cancer Res., 63(18):5821-8, 2003.
Sinner et al., Mol. Cell Biol., 27(22):7802-15, 2007.
Stone et al., Int. J. Cancer, 21:274-281, 1978
Tang et al., Cancer Res., 67(18):8643-52, 2007.
Tang et al., Mol. Carcinog., 46(1):1-14, 2007.
Thiyagarajan et al., Cancer Res., 67(22):10642-6, 2007.
UK 8 803 000
Vezina & Bushman, Curr. Urol. Rep., 8(4): 275-80, 2007.
Wang et al., Proc. Natl. Acad. Sci. USA, 103(5):1480-1485, 2006.
Watabe et al., Proc Natl Acad Sci USA, 2002 Jan. 8;99(1):401-6, 2002.
Willert et al., Nature, 423(6938):448-52, 2003.
Xie et al., Cancer, 97(9):2229-35, 2007.
Yang et al., Cancer Cell, 13(2):153-66, 2008.
Yu et al., Cell, 131(6):1109-23, 2007.
Zhang et al., Cancer Res., 68(11):4311-20, 2008.
Zhang et al., FEBS J., 273(8):1723-30, 2006.
Zhou et al., Nat. Med., 7(9):1028-1034, 2001.
Zhou et al., Proc. Natl. Acad. Sci. USA, 104(41):16158-63, 2007.

1. A method of treating cancer comprising administering to a subject in need of such therapy an effective amount of:
(a) an inhibitor of hsa-let-7b, hsa-let-7c, hsa-let-7e, hsa-miR-100, hsa-miR-101, hsa-miR-105, hsa-miR-10a, hsa-miR-10b, hsa-miR-124a, hsa-miR-125a, hsa-miR-125b, hsa-miR-127, hsa-miR-133a, hsa-miR-133b, hsa-miR-140, hsa-miR-142-3p, hsa-miR-142-5p, hsa-miR-145, hsa-miR-146a, hsa-miR-146b, hsa-miR-147, hsa-miR-148a, hsa-miR-148b, hsa-miR-15b, hsa-miR-16, hsa-miR-181a, hsa-miR-181d, hsa-miR-183, hsa-miR-18a, hsa-miR-190, hsa-miR-192, hsa-miR-193b, hsa-miR-195, hsa-miR-196a, hsa-miR-196b, hsa-miR-19a, hsa-miR-19b, hsa-miR-203, hsa-miR-205, hsa-miR-21, hsa-miR-214, hsa-miR-218, hsa-miR-22, hsa-miR-221, hsa-miR-222, hsa-miR-223, hsa-miR-24, hsa-miR-25, hsa-miR-26b, hsa-miR-27a, hsa-miR-27b, hsa-miR-28, hsa-miR-29a, hsa-miR-29b, hsa-miR-29c, hsa-miR-301, hsa-miR-30a-3p, hsa-miR-30a-5p, hsa-miR-30b, hsa-miR-30c, hsa-miR-30d, hsa-miR-30e-5p, hsa-miR-32, hsa-miR-320, hsa-miR-324-3p, hsa-miR-324-5p, hsa-miR-328, hsa-miR-33, hsa-miR-331, hsa-miR-338, hsa-miR-339, hsa-miR-345, hsa-miR-361, hsa-miR-374, hsa-miR-378, hsa-miR-422a, hsa-miR-422b, hsa-miR-423, hsa-miR-425, hsa-miR-433, hsa-miR-451, hsa-miR-452, hsa-miR-500, hsa-miR-501, hsa-miR-9, and/or hsa-miR-96; or
(b) a molecule having hsa-let-7a, hsa-let-7b, hsa-let-7c, hsa-let-7e, hsa-let-7f, hsa-let-7i, hsa-miR-101, hsa-miR-103, hsa-miR-106a, hsa-miR-10a, hsa-miR-10b, hsa-miR-124a, hsa-miR-125a, hsa-miR-126, hsa-miR-132, hsa-miR-133a, hsa-miR-141, hsa-miR-146a, hsa-miR-146b, hsa-miR-148a, hsa-miR-148b, hsa-miR-149, hsa-miR-151, hsa-miR-152, hsa-miR-15a, hsa-miR-15b, hsa-miR-181b, hsa-miR-182, hsa-miR-183, hsa-miR-18a, hsa-miR-191, hsa-miR-193b, hsa-miR-195, hsa-miR-196a, hsa-miR-196b, hsa-miR-199a, hsa-miR-200a, hsa-miR-200c, hsa-miR-203, hsa-miR-205, hsa-miR-21, hsa-miR-214, hsa-miR-218, hsa-miR-22, hsa-miR-222, hsa-miR-24, hsa-miR-25, hsa-miR-26a, hsa-miR-27a, hsa-miR-29b, hsa-miR-29c, hsa-miR-30a-3p, hsa-miR-30a-5p, hsa-miR-30d, hsa-miR-320, hsa-miR-324-5p, hsa-miR-328, hsa-miR-331, hsa-miR-335, hsa-miR-340, hsa-miR-342, hsa-miR-345, hsa-miR-34a, hsa-miR-362, hsa-miR-365, hsa-miR-375, hsa-miR-378, hsa-miR-422a, hsa-miR-422b, hsa-miR-425, hsa-miR-429, hsa-miR-500, hsa-miR-7, hsa-miR-9*, hsa-miR-92, hsa-miR-93, hsa-miR-98, and/or hsa-miR-99b activity.

2. The method of claim 1, wherein the nucleic acid inhibitor is an inhibitor of hsa-miR-125b, hsa-miR-133b, hsa-miR-142-3p, hsa-miR-181a, hsa-miR-181d, hsa-miR-205, hsa-miR-221, hsa-miR-222, hsa-miR-223, hsa-miR-29a, hsa-miR-29c, hsa-miR-301, hsa-miR-328, hsa-miR-33, hsa-miR-338, hsa-miR-374, hsa-miR-378, hsa-miR-451, hsa-miR-452, and/or hsa-miR-501.

3. The method of claim 1, wherein the nucleic acid has an activity of one or more hsa-let-7c, hsa-miR-126, hsa-miR-15a, hsa-miR-15b, hsa-miR-182, hsa-miR-183, hsa-miR-191, hsa-miR-200a, hsa-miR-200c, hsa-miR-203, hsa-miR-218, hsa-miR-27a, hsa-miR-29b, hsa-miR-30a-3p, hsa-miR-30a-5p, hsa-miR-335, hsa-miR-340, hsa-miR-34a, hsa-miR-365, hsa-miR-375, hsa-miR-422a, hsa-miR-425, hsa-miR-92, and/or hsa-miR-93.

4. The method of claim 1, wherein a nucleic acid has an activity of a hsa-let-7b, hsa-miR-34a, hsa-miR-126, hsa-miR-141, hsa-miR-335, hsa-miR-365, and/or hsa-miR-375.

5. The method of claim 1, wherein a nucleic acid inhibitor is an inhibitor of hsa-miR-142-3p, hsa-miR-33, hsa-miR-301, hsa-miR-451, and/or hsa-miR-452.

6. The method of claim 1, wherein a hsa-miR-34a, hsa-miR-126 and/or hsa-let-7b nucleic acid is administered.

7. (canceled)

8. The method of claim 1, wherein the subject is suspected of having or at risk of developing brain, breast, bone, colorectal, pancreatic, ovarian, prostate, testicular, hepatic, kidney, or lung cancer.

9. The method of claim 8, wherein the subject is suspected of or at risk of developing prostate cancer.

10. (canceled)

11. (canceled)

12. (canceled)

13. (canceled)

14. (canceled)

15. (canceled)

16. The method of claim 1, wherein the therapeutic nucleic acid is a synthetic nucleic acid.

17. The method of claim 16, wherein the nucleic acid is administered at a dose of 0.01 μg/kg of body weight to 10 mg/kg of body weight.

18. (canceled)

19. (canceled)

20. (canceled)

21. (canceled)

22. (canceled)

23. The method of claim 1, further comprising administering 2, 3, 4, 5, 6, or more therapeutic nucleic acids.

24. (canceled)

25. The method of claim 1, wherein proliferation of the cancer is reduced.

26. The method of claim 1, wherein metastasis of the cancer is reduced.

27. The method of claim 1, wherein the cancer is a metastasized cancer.

28. (canceled)

29. (canceled)

30. (canceled)

31. A method of treating a patient diagnosed with or suspected of having or at risk of developing prostate cancer comprising the step of administering to the patient an effective amount of:
(a) an inhibitor of one or more of hsa-let-7b, hsa-let-7c, hsa-let-7e, hsa-miR-100, hsa-miR-101, hsa-miR-105, hsa-miR-10a, hsa-miR-10b, hsa-miR-124a, hsa-miR-125a, hsa-miR-125b, hsa-miR-127, hsa-miR-133a, hsa-miR-133b, hsa-miR-140, hsa-miR-142-3p, hsa-miR-142-5p, hsa-miR-145, hsa-miR-146a, hsa-miR-146b, hsa-miR-147, hsa-miR-148a, hsa-miR-148b, hsa-miR-15b, hsa-miR-16, hsa-miR-181a, hsa-miR-181d, hsa-miR-183, hsa-miR-18a, hsa-miR-190, hsa-miR-192, hsa-miR-193b, hsa-miR-195, hsa-miR-196a, hsa-miR-196b, hsa-miR-19a, hsa-miR-19b, hsa-miR-203, hsa-miR-205, hsa-miR-21, hsa-miR-214, hsa-miR-218, hsa-miR-22, hsa-miR-221, hsa-miR-222, hsa-miR-223, hsa-miR-24, hsa-miR-25, hsa-miR-26b, hsa-miR-27a, hsa-miR-27b, hsa-miR-28, hsa-miR-29a, hsa-miR-29b, hsa-miR-29c, hsa-miR-301, hsa-miR-30a-3p, hsa-miR-30a-5p, hsa-miR-30b, hsa-miR-30c, hsa-miR-30d, hsa-miR-30e-5p, hsa-miR-32, hsa-miR-320, hsa-miR-324-3p, hsa-miR-324-5p, hsa-miR-328, hsa-miR-33, hsa-miR-331, hsa-miR-338, hsa-miR-339, hsa-miR-345, hsa-miR-361, hsa-miR-374, hsa-miR-378, hsa-miR-422a, hsa-miR-422b, hsa-miR-423, hsa-miR-425, hsa-miR-433, hsa-miR-451, hsa-miR-452, hsa-miR-500, hsa-miR-501, hsa-miR-9, and/or hsa-miR-96; and/or
(b) a molecule having an activity of one or more of hsa-let-7a, hsa-let-7b, hsa-let-7c, hsa-let-7e, hsa-let-7f, hsa-let-7i, hsa-miR-101, hsa-miR-103, hsa-miR-106a, hsa-miR-10a, hsa-miR-10b, hsa-miR-124a, hsa-miR-125a, hsa-miR-126, hsa-miR-132, hsa-miR-133a, hsa-miR-141, hsa-miR-146a, hsa-miR-146b, hsa-miR-148a, hsa-miR-148b, hsa-miR-149, hsa-miR-151, hsa-miR-152, hsa-miR-15a, hsa-miR-15b, hsa-miR-181b, hsa-miR-182, hsa-miR-183, hsa-miR-18a, hsa-miR-191, hsa-miR-193b, hsa-miR-195, hsa-miR-196a, hsa-miR-196b, hsa-miR-199a, hsa-miR-200a, hsa-miR-200c, hsa-miR-203, hsa-miR-205, hsa-miR-21, hsa-miR-214, hsa-miR-218, hsa-miR-22, hsa-miR-222, hsa-miR-24, hsa-miR-25, hsa-miR-26a, hsa-miR-27a, hsa-miR-29b, hsa-miR-29c, hsa-miR-30a-3p, hsa-miR-30a-5p, hsa-miR-30d, hsa-miR-320, hsa-miR-324-5p, hsa-miR-328, hsa-miR-331, hsa-miR-335, hsa-miR-340, hsa-miR-342, hsa-miR-345, hsa-miR-34a, hsa-miR-362, hsa-miR-365, hsa-miR-375, hsa-miR-378, hsa-miR-422a, hsa-miR-422b, hsa-miR-425, hsa-miR-429, hsa-miR-500, hsa-miR-7, hsa-miR-9*, hsa-miR-92, hsa-miR-93, hsa-miR-98, and/or hsa-miR-99b activity.

32. (canceled)

33. (canceled)

34. (canceled)

35. (canceled)

36. (canceled)

37. (canceled)

38. The method of claim 31, wherein the therapeutic nucleic acid is a synthetic nucleic acid.

39. The method of claim 38, wherein the nucleic acid is administered at a dose of 0.01 μg/kg of body weight to 10 mg/kg of body weight.

40. (canceled)

41. (canceled)

42. (canceled)

43. (canceled)

44. (canceled)

45. (canceled)

46. (canceled)

47. The method of claim 31, wherein proliferation of the cancer is reduced.

48. The method of claim 31, wherein metastasis of the cancer is reduced.

49. (canceled)

50. (canceled)

51. (canceled)

52. (canceled)

53. (canceled)

54. A method of detecting cancer stem cells in a biological sample comprising evaluating expression levels of hsa-let-7a, hsa-let-7b, hsa-let-7c, hsa-let-7e, hsa-let-7f, hsa-let-7i, hsa-miR-100, hsa-miR-101, hsa-miR-103, hsa-miR-105, hsa-miR-106a, hsa-miR-10a, hsa-miR-10b, hsa-miR-124a, hsa-miR-125a, hsa-miR-125b, hsa-miR-126, hsa-miR-127, hsa-miR-132, hsa-miR-133a, hsa-miR-133b, hsa-miR-140, hsa-miR-141, hsa-miR-142-3p, hsa-miR-142-5p, hsa-miR-145, hsa-miR-146a, hsa-miR-146b, hsa-miR-147, hsa-miR-148a, hsa-miR-148b, hsa-miR-149, hsa-miR-151, hsa-miR-152, hsa-miR-15a, hsa-miR-15b, hsa-miR-16, hsa-miR-181a, hsa-miR-181b, hsa-miR-181d, hsa-miR-182, hsa-miR-183, hsa-miR-18a, hsa-miR-190, hsa-miR-191, hsa-miR-192, hsa-miR-193b, hsa-miR-195, hsa-miR-196a, hsa-miR-196b, hsa-miR-199a, hsa-miR-19a, hsa-miR-19b, hsa-miR-200a, hsa-miR-200c, hsa-miR-203, hsa-miR-205, hsa-miR-21, hsa-miR-214, hsa-miR-218, hsa-miR-22, hsa-miR-221, hsa-miR-222, hsa-miR-223, hsa-miR-24, hsa-miR-25, hsa-miR-26a, hsa-miR-26b, hsa-miR-27a, hsa-miR-27b, hsa-miR-28, hsa-miR-29a, hsa-miR-29b, hsa-miR-29c, hsa-miR-301, hsa-miR-30a-3p, hsa-miR-30a-5p, hsa-miR-30b, hsa-miR-30c, hsa-miR-30d, hsa-miR-30e-5p, hsa-miR-32, hsa-miR-320, hsa-miR-324-3p, hsa-miR-324-5p, hsa-miR-328, hsa-miR-33, hsa-miR-331, hsa-miR-335, hsa-miR-338, hsa-miR-339, hsa-miR-340, hsa-miR-342, hsa-miR-345, hsa-miR-34a, hsa-miR-361, hsa-miR-362, hsa-miR-365, hsa-miR-374, hsa-miR-375, hsa-miR-378, hsa-miR-422a, hsa-miR-422b, hsa-miR-423, hsa-miR-425, hsa-miR-429, hsa-miR-433, hsa-miR-451, hsa-miR-452, hsa-miR-500, hsa-miR-501, hsa-miR-7, hsa-miR-9, hsa-miR-9*, hsa-miR-92, hsa-miR-93, hsa-miR-96, hsa-miR-98, hsa-miR-99b, or combinations thereof.

55. (canceled)

56. The method of claim 54, wherein an increase in the level of one or more of hsa-let-7b, hsa-let-7c, hsa-let-7e, hsa-miR-100, hsa-miR-101, hsa-miR-105, hsa-miR-10a, hsa-miR-10b, hsa-miR-124a, hsa-miR-125a, hsa-miR-125b, hsa-miR-127, hsa-miR-133a, hsa-miR-133b, hsa-miR-140, hsa-miR-142-3p, hsa-miR-142-5p, hsa-miR-145, hsa-miR-146a, hsa-miR-146b, hsa-miR-147, hsa-miR-148a, hsa-miR-148b, hsa-miR-15b, hsa-miR-16, hsa-miR-181a, hsa-miR-181d, hsa-miR-183, hsa-miR-18a, hsa-miR-190, hsa-miR-192, hsa-miR-193b, hsa-miR-195, hsa-miR-196a, hsa-miR-196b, hsa-miR-19a, hsa-miR-19b, hsa-miR-203, hsa-miR-205, hsa-miR-21, hsa-miR-214, hsa-miR-218, hsa-miR-22, hsa-miR-221, hsa-miR-222, hsa-miR-223, hsa-miR-24, hsa-miR-25, hsa-miR-26b, hsa-miR-27a, hsa-miR-27b, hsa-miR-28, hsa-miR-29a, hsa-miR-29b, hsa-miR-29c, hsa-miR-301, hsa-miR-30a-3p, hsa-miR-30a-5p, hsa-miR-30b, hsa-miR-30c, hsa-miR-30d, hsa-miR-30e-5p, hsa-miR-32, hsa-miR-320, hsa-miR-324-3p, hsa-miR-324-5p, hsa-miR-328, hsa-miR-33, hsa-miR-331, hsa-miR-338, hsa-miR-339, hsa-miR-345, hsa-miR-361, hsa-miR-374, hsa-miR-378, hsa-miR-422a, hsa-miR-422b, hsa-miR-423, hsa-miR-425, hsa-miR-433, hsa-miR-451, hsa-miR-452, hsa-miR-500, hsa-miR-501, hsa-miR-9, and/or hsa-miR-96 is indicative of a cancer stem cell.

57. The method of claim 54, wherein an decrease in the level of one or more of hsa-let-7a, hsa-let-7b, hsa-let-7c, hsa-let-7e, hsa-let-7f, hsa-let-7i, hsa-miR-101, hsa-miR-103, hsa-miR-106a, hsa-miR-10a, hsa-miR-10b, hsa-miR-124a, hsa-miR-125a, hsa-miR-126, hsa-miR-132, hsa-miR-133a, hsa-miR-141, hsa-miR-146a, hsa-miR-146b, hsa-miR-148a, hsa-miR-148b, hsa-miR-149, hsa-miR-151, hsa-miR-152, hsa-miR-15a, hsa-miR-15b, hsa-miR-181b, hsa-miR-182, hsa-miR-183, hsa-miR-18a, hsa-miR-191, hsa-miR-193b, hsa-miR-195, hsa-miR-196a, hsa-miR-196b, hsa-miR-199a, hsa-miR-200a, hsa-miR-200c, hsa-miR-203, hsa-miR-205, hsa-miR-21, hsa-miR-214, hsa-miR-218, hsa-miR-22, hsa-miR-222, hsa-miR-24, hsa-miR-25, hsa-miR-26a, hsa-miR-27a, hsa-miR-29b, hsa-miR-29c, hsa-miR-30a-3p, hsa-miR-30a-5p, hsa-miR-30d, hsa-miR-320, hsa-miR-324-5p, hsa-miR-328, hsa-miR-331, hsa-miR-335, hsa-miR-340, hsa-miR-342, hsa-miR-345, hsa-miR-34a, hsa-miR-362, hsa-miR-365, hsa-miR-375, hsa-miR-378, hsa-miR-422a, hsa-miR-422b, hsa-miR-425, hsa-miR-429, hsa-miR-500, hsa-miR-7, hsa-miR-9*, hsa-miR-92, hsa-miR-93, hsa-miR-98, and/or hsa-miR-99b is indicative of a cancer stem cell.


SEQUENCE LISTING (120 sequences)

SEQ ID NO: 1 - RNA, 22 nt, Homo sapiens
ugagguaguagguuguauaguu

SEQ ID NO: 2 - RNA, 22 nt, Homo sapiens
ugagguaguagguugugugguu

SEQ ID NO: 3 - RNA, 22 nt, Homo sapiens
ugagguaguagguuguaugguu

SEQ ID NO: 4 - RNA, 22 nt, Homo sapiens
ugagguaggagguuguauaguu

SEQ ID NO: 5 - RNA, 22 nt, Homo sapiens
ugagguaguagauuguauaguu

SEQ ID NO: 6 - RNA, 22 nt, Homo sapiens
ugagguaguaguuugugcuguu

SEQ ID NO: 7 - RNA, 22 nt, Homo sapiens
aacccguagauccgaacuugug

SEQ ID NO: 8 - RNA, 21 nt, Homo sapiens
uacaguacugugauaacugaa

SEQ ID NO: 9 - RNA, 23 nt, Homo sapiens
agcagcauuguacagggcuauga

SEQ ID NO: 10 - RNA, 23 nt, Homo sapiens
ucaaaugcucagacuccuguggu

SEQ ID NO: 11 - RNA, 23 nt, Homo sapiens
aaaagugcuuacagugcagguag

SEQ ID NO: 12 - RNA, 23 nt, Homo sapiens
uacccuguagauccgaauuugug

SEQ ID NO: 13 - RNA, 23 nt, Homo sapiens
uacccuguagaaccgaauuugug

SEQ ID NO: 14 - RNA, 20 nt, Homo sapiens
uaaggcacgcggugaaugcc

SEQ ID NO: 15 - RNA, 24 nt, Homo sapiens
ucccugagacccuuuaaccuguga

SEQ ID NO: 16 - RNA, 22 nt, Homo sapiens
ucccugagacccuaacuuguga

SEQ ID NO: 17 - RNA, 22 nt, Homo sapiens
ucguaccgugaguaauaaugcg

SEQ ID NO: 18 - RNA, 22 nt, Homo sapiens
ucggauccgucugagcuuggcu

SEQ ID NO: 19 - RNA, 22 nt, Homo sapiens
uaacagucuacagccauggucg

SEQ ID NO: 20 - RNA, 22 nt, Homo sapiens
uuugguccccuucaaccagcug

SEQ ID NO: 21 - RNA, 22 nt, Homo sapiens
uuugguccccuucaaccagcua

SEQ ID NO: 22 - RNA, 22 nt, Homo sapiens
cagugguuuuacccuaugguag

SEQ ID NO: 23 - RNA, 22 nt, Homo sapiens
uaacacugucugguaaagaugg

SEQ ID NO: 24 - RNA, 23 nt, Homo sapiens
uguaguguuuccuacuuuaugga

SEQ ID NO: 25 - RNA, 21 nt, Homo sapiens
cauaaaguagaaagcacuacu

SEQ ID NO: 26 - RNA, 23 nt, Homo sapiens
guccaguuuucccaggaaucccu

SEQ ID NO: 27 - RNA, 22 nt, Homo sapiens
ugagaacugaauuccauggguu

SEQ ID NO: 28 - RNA, 22 nt, Homo sapiens
ugagaacugaauuccauaggcu

SEQ ID NO: 29 - RNA, 20 nt, Homo sapiens
guguguggaaaugcuucugc

SEQ ID NO: 30 - RNA, 22 nt, Homo sapiens
ucagugcacuacagaacuuugu

SEQ ID NO: 31 - RNA, 22 nt, Homo sapiens
ucagugcaucacagaacuuugu

SEQ ID NO: 32 - RNA, 23 nt, Homo sapiens
ucuggcuccgugucuucacuccc

SEQ ID NO: 33 - RNA, 21 nt, Homo sapiens
ucgaggagcucacagucuagu

SEQ ID NO: 34 - RNA, 21 nt, Homo sapiens
ucagugcaugacagaacuugg

SEQ ID NO: 35 - RNA, 22 nt, Homo sapiens
uagcagcacauaaugguuugug

SEQ ID NO: 36 - RNA, 22 nt, Homo sapiens
uagcagcacaucaugguuuaca

SEQ ID NO: 37 - RNA, 22 nt, Homo sapiens
uagcagcacguaaauauuggcg

SEQ ID NO: 38 - RNA, 23 nt, Homo sapiens
aacauucaacgcugucggugagu

SEQ ID NO: 39 - RNA, 23 nt, Homo sapiens
aacauucauugcugucggugggu

SEQ ID NO: 40 - RNA, 23 nt, Homo sapiens
aacauucauuguugucggugggu

SEQ ID NO: 41 - RNA, 24 nt, Homo sapiens
uuuggcaaugguagaacucacacu

SEQ ID NO: 42 - RNA, 22 nt, Homo sapiens
uauggcacugguagaauucacu

SEQ ID NO: 43 - RNA, 23 nt, Homo sapiens
uaaggugcaucuagugcagauag

SEQ ID NO: 44 - RNA, 22 nt, Homo sapiens
ugauauguuugauauauuaggu

SEQ ID NO: 45 - RNA, 23 nt, Homo sapiens
caacggaaucccaaaagcagcug

SEQ ID NO: 46 - RNA, 21 nt, Homo sapiens
cugaccuaugaauugacagcc

SEQ ID NO: 47 - RNA, 22 nt, Homo sapiens
aacuggcccucaaagucccgcu

SEQ ID NO: 48 - RNA, 21 nt, Homo sapiens
uagcagcacagaaauauuggc

SEQ ID NO: 49 - RNA, 22 nt, Homo sapiens
uagguaguuucauguuguuggg

SEQ ID NO: 50 - RNA, 22 nt, Homo sapiens
uagguaguuuccuguuguuggg

SEQ ID NO: 51 - RNA, 23 nt, Homo sapiens
cccaguguucagacuaccuguuc

SEQ ID NO: 52 - RNA, 23 nt, Homo sapiens
ugugcaaaucuaugcaaaacuga

SEQ ID NO: 53 - RNA, 23 nt, Homo sapiens
ugugcaaauccaugcaaaacuga

SEQ ID NO: 54 - RNA, 22 nt, Homo sapiens
uaacacugucugguaacgaugu

SEQ ID NO: 55 - RNA, 23 nt, Homo sapiens
uaauacugccggguaaugaugga

SEQ ID NO: 56 - RNA, 22 nt, Homo sapiens
gugaaauguuuaggaccacuag

SEQ ID NO: 57 - RNA, 22 nt, Homo sapiens
uccuucauuccaccggagucug

SEQ ID NO: 58 - RNA, 22 nt, Homo sapiens
uagcuuaucagacugauguuga

SEQ ID NO: 59 - RNA, 22 nt, Homo sapiens
acagcaggcacagacaggcagu

SEQ ID NO: 60 - RNA, 21 nt, Homo sapiens
uugugcuugaucuaaccaugu

SEQ ID NO: 61 - RNA, 22 nt, Homo sapiens
aagcugccaguugaagaacugu

SEQ ID NO: 62 - RNA, 23 nt, Homo sapiens
agcuacauugucugcuggguuuc

SEQ ID NO: 63 - RNA, 21 nt, Homo sapiens
agcuacaucuggcuacugggu

SEQ ID NO: 64 - RNA, 22 nt, Homo sapiens
ugucaguuugucaaauacccca

SEQ ID NO: 65 - RNA, 22 nt, Homo sapiens
uggcucaguucagcaggaacag

SEQ ID NO: 66 - RNA, 22 nt, Homo sapiens
cauugcacuugucucggucuga

SEQ ID NO: 67 - RNA, 22 nt, Homo sapiens
uucaaguaauccaggauaggcu

SEQ ID NO: 68 - RNA, 21 nt, Homo sapiens
uucaaguaauucaggauaggu

SEQ ID NO: 69 - RNA, 21 nt, Homo sapiens
uucacaguggcuaaguuccgc

SEQ ID NO: 70 - RNA, 21 nt, Homo sapiens
uucacaguggcuaaguucugc

SEQ ID NO: 71 - RNA, 22 nt, Homo sapiens
aaggagcucacagucuauugag

SEQ ID NO: 72 - RNA, 22 nt, Homo sapiens
uagcaccaucugaaaucgguua

SEQ ID NO: 73 - RNA, 23 nt, Homo sapiens
uagcaccauuugaaaucaguguu

SEQ ID NO: 74 - RNA, 22 nt, Homo sapiens
uagcaccauuugaaaucgguua

SEQ ID NO: 75 - RNA, 23 nt, Homo sapiens
cagugcaauaguauugucaaagc

SEQ ID NO: 76 - RNA, 22 nt, Homo sapiens
cuuucagucggauguuugcagc

SEQ ID NO: 77 - RNA, 22 nt, Homo sapiens
uguaaacauccucgacuggaag

SEQ ID NO: 78 - RNA, 22 nt, Homo sapiens
uguaaacauccuacacucagcu

SEQ ID NO: 79 - RNA, 23 nt, Homo sapiens
uguaaacauccuacacucucagc

SEQ ID NO: 80 - RNA, 22 nt, Homo sapiens
uguaaacauccccgacuggaag

SEQ ID NO: 81 - RNA, 22 nt, Homo sapiens
uguaaacauccuugacuggaag

SEQ ID NO: 82 - RNA, 22 nt, Homo sapiens
uauugcacauuacuaaguugca

SEQ ID NO: 83 - RNA, 22 nt, Homo sapiens
aaaagcuggguugagagggcga

SEQ ID NO: 84 - RNA, 20 nt, Homo sapiens
acugccccaggugcugcugg

SEQ ID NO: 85 - RNA, 23 nt, Homo sapiens
cgcauccccuagggcauuggugu

SEQ ID NO: 86 - RNA, 22 nt, Homo sapiens
cuggcccucucugcccuuccgu

SEQ ID NO: 87 - RNA, 21 nt, Homo sapiens
gugcauuguaguugcauugca

SEQ ID NO: 88 - RNA, 22 nt, Homo sapiens
cuagguauggucccagggaucc

SEQ ID NO: 89 - RNA, 23 nt, Homo sapiens
ucaagagcaauaacgaaaaaugu

SEQ ID NO: 90 - RNA, 22 nt, Homo sapiens
aacaauauccuggugcugagug

SEQ ID NO: 91 - RNA, 23 nt, Homo sapiens
ucccuguccuccaggagcucacg

SEQ ID NO: 92 - RNA, 22 nt, Homo sapiens
uuauaaagcaaugagacugauu

SEQ ID NO: 93 - RNA, 21 nt, Homo sapiens
aggggugcuaucugugauuga

SEQ ID NO: 94 - RNA, 22 nt, Homo sapiens
gcugacuccuaguccagggcuc

SEQ ID NO: 95 - RNA, 22 nt, Homo sapiens
uggcagugucuuagcugguugu

SEQ ID NO: 96 - RNA, 22 nt, Homo sapiens
uuaucagaaucuccagggguac

SEQ ID NO: 97 - RNA, 24 nt, Homo sapiens
aauccuuggaaccuaggugugagu

SEQ ID NO: 98 - RNA, 22 nt, Homo sapiens
uaaugccccuaaaaauccuuau

SEQ ID NO: 99 - RNA, 22 nt, Homo sapiens
uuauaauacaaccugauaagug

SEQ ID NO: 100 - RNA, 22 nt, Homo sapiens
uuuguucguucggcucgcguga

SEQ ID NO: 101 - RNA, 21 nt, Homo sapiens
acuggacuuggagucagaagg

SEQ ID NO: 102 - RNA, 22 nt, Homo sapiens
acuggacuuagggucagaaggc

SEQ ID NO: 103 - RNA, 21 nt, Homo sapiens
acuggacuuggagucagaagg

SEQ ID NO: 104 - RNA, 23 nt, Homo sapiens
ugaggggcagagagcgagacuuu

SEQ ID NO: 105 - RNA, 23 nt, Homo sapiens
aaugacacgaucacucccguuga

SEQ ID NO: 106 - RNA, 22 nt, Homo sapiens
uaauacugucugguaaaaccgu

SEQ ID NO: 107 - RNA, 22 nt, Homo sapiens
aucaugaugggcuccucggugu

SEQ ID NO: 108 - RNA, 22 nt, Homo sapiens
aaaccguuaccauuacugaguu

SEQ ID NO: 109 - RNA, 22 nt, Homo sapiens
aacuguuugcagaggaaacuga

SEQ ID NO: 110 - RNA, 23 nt, Homo sapiens
uaauccuugcuaccugggugaga

SEQ ID NO: 111 - RNA, 22 nt, Homo sapiens
aauccuuugucccugggugaga

SEQ ID NO: 112 - RNA, 23 nt, Homo sapiens
uggaagacuagugauuuuguugu

SEQ ID NO: 113 - RNA, 22 nt, Homo sapiens
auaaagcuagauaaccgaaagu

SEQ ID NO: 114 - RNA, 23 nt, Homo sapiens
ucuuugguuaucuagcuguauga

SEQ ID NO: 115 - RNA, 22 nt, Homo sapiens
uauugcacuugucccggccugu

SEQ ID NO: 116 - RNA, 23 nt, Homo sapiens
caaagugcuguucgugcagguag

SEQ ID NO: 117 - RNA, 23 nt, Homo sapiens
uuuggcacuagcacauuuuugcu

SEQ ID NO: 118 - RNA, 22 nt, Homo sapiens
ugagguaguaaguuguauuguu

SEQ ID NO: 119 - RNA, 22 nt, Homo sapiens
cacccguagaaccgaccuugcg

SEQ ID NO: 120 - protein, 4 aa, Artificial
  Synthetic peptide
KDEL